(19) United States
(12) Patent Application Publication
(10) Pub. No.: US 2026/0265238 A1
(43) Pub. Date: Sep. 10, 2026

(54) COMPOUNDS AND PHARMACEUTICAL COMPOSITIONS THAT DEGRADE SWI/SNF-RELATED MATRIX-ASSOCIATED ACTIN-DEPENDENT REGULATOR OF CHROMATIN SUBFAMILY A

(71) Applicant: Plexium, Inc., San Diego, CA (US)

(72) Inventors: Ronghua Li, San Diego, CA (US); Simon Bailey, San Diego, CA (US); Geoffray Leriche, San Diego, CA (US); Dange Vijay Kumar, San Diego, CA (US); Aaron C. Burns, San Diego, CA (US); Kevin D. Freeman-Cook, Carlsbad, CA (US); Michael J. Bishop, San Diego, CA (US)

(21) Appl. No.: 18/875,070

(22) PCT Filed: Jun. 16, 2023
(86) PCT No.: PCT/US2023/025576

§ 371 Date: Dec. 13, 2024

(51) Int. Cl. C07D 413/14; A61K 31/501; A61K 31/506; A61K 31/519; A61K 31/5365; A61K 31/5377; A61K 31/5383; A61K 31/5386; A61K 31/541; A61K 31/551; A61K 31/553; C07D 401/14; C07D 403/12; C07D 403/14; C07D 405/14; C07D 417/14; C07D 471/04; C07D 471/08; C07D 471/10; C07D 487/04; C07D 487/08; C07D 491/107; C07D 498/04; C07D 498/08; C07D 519/00

(52) U.S. Cl. CPC C07D 413/14; A61K 31/501; A61K 31/506; A61K 31/519; A61K 31/5365; A61K 31/5377; A61K 31/5383; A61K 31/5386; A61K 31/541; A61K 31/551; A61K 31/553; C07D 401/14; C07D 403/12; C07D 403/14; C07D 405/14; C07D 417/14; C07D 471/04; C07D 471/08; C07D 471/10; C07D 487/04; C07D 487/08; C07D 491/107; C07D 498/04; C07D 498/08; C07D 519/00

(57) ABSTRACT

Disclosed are compounds of formula I, or a pharmaceutically acceptable salt, solvate, stereoisomer, or tautomer thereof, that are useful for modulating or degrading protein which is expressed from one or more SWI/SNF-related matrix-associated actin-dependent regulator of chromatin subfamily A (SMARCA) (e.g., SMAR-CA2 and/or SMARCA4).

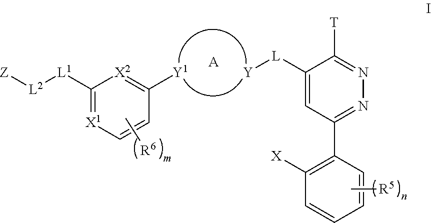

### CROSS-REFERENCE TO RELATED APPLICATIONS

This application claims the benefit under 35 U.S.C. § 119(e) of U.S. Provisional Application Ser. No. 63/352,936, filed Jun. 16, 2022, the contents of which are hereby incorporated by reference in its entirety.

### FIELD

This disclosure provides for compounds, including pharmaceutically acceptable salts thereof, that are useful as modulators of targeted ubiquitination. The compounds disclosed herein bind to and degrade protein which is expressed from one or more SWI/SNF-related matrix-associated actin-dependent regulator of chromatin subfamily A (“SMARCA”). Also disclosed are pharmaceutical compositions comprising the compounds, and methods of using such compounds in the treatment of various SMARCA-mediated diseases or disorders.

### STATE OF THE ART

Ubiquitin-Proteasome Pathway (UPP) is a critical pathway that regulates key regulator proteins and degrades misfolded or abnormal proteins. UPP is central to multiple cellular processes, and if defective or imbalanced, it leads to pathogenesis of a variety of diseases. The covalent attachment of ubiquitin to specific protein substrates is achieved through the action of E3 ubiquitin ligases.

There are over 600 E3 ubiquitin ligases which facilitate the ubiquitination of different proteins in vivo, which can be divided into four families: HECT-domain E3s, U-box E3s, monomeric RING E3s and multi-subunit E3s. See e.g., Li et al. “Genome-wide and functional annotation of human E3 ubiquitin ligases identifies MULAN, a mitochondrial E3 that regulates the organelle's dynamics and signaling.” PLOS One 2008, (3) 1487; Berndsen et al. “New insights into ubiquitin E3 ligase mechanism” Nat. Struct. Mol. Biol. 2014, 21:301; Deshaies et al. “RING domain E3 ubiquitin ligases” Ann. Rev. Biochem. 2009, 78:399; Sprattetal. “RBRE3 ubiquitin ligases: new structures, new insights, new questions” Biochem. 2014, 458:421; and Wang et al., “Roles of F-box proteins in cancer” Nat. Rev. Cancer. 2014, 14:233.

UPP plays a key role in the degradation of short-lived and regulatory proteins important in a variety of basic cellular processes, including regulation of the cell cycle, modulation of cell surface receptors and ion channels, and antigen presentation. The pathway has been implicated in several forms of malignancy, in the pathogenesis of several genetic diseases (including cystic fibrosis, Angelman's syndrome, and Liddle syndrome), in immune surveillance/viral pathogenesis, and in the pathology of muscle wasting. Many diseases are associated with an abnormal UPP and negatively affect cell cycle and division, the cellular response to stress and to extracellular modulators, morphogenesis of neuronal networks, modulation of cell surface receptors, ion channels, the secretory pathway, DNA repair, and biogenesis of organelles.

Aberrations in the process have recently been implicated in the pathogenesis of several diseases, both inherited and acquired. These diseases fall in to two major groups: (a) those that result from loss of function with the resultant stabilization of certain proteins, and (b) those that result from gain of function, i.e. abnormal or accelerated degradation of the protein target.

The UPP is used to induce selective protein degradation, including use of fusion proteins to artificially ubiquitinate target proteins and synthetic small-molecule probes to induce proteasome-dependent degradation. Compounds that act as molecular glues can induce or stabilize protein-protein interactions between a target protein and an E3 ubiquitin ligase ligand, leading to protein ubiquitination and subsequent proteasome-mediated degradation via the recruitment to E3 ubiquitin ligase and subsequent ubiquitination. These drug-like molecules offer the possibility of temporal control over protein expression. Such compounds are capable of inducing the inactivation of a protein of interest upon addition to cells or administration to an animal or human, and could be useful as biochemical reagents and lead to a new paradigm for the treatment of diseases by removing pathogenic or oncogenic proteins. See e.g., Crews, Chem. & Biol. 2010, 17 (6): 551; Schneekloth and Crews, Chem Bio Chem., 2005, 6 (1): 40.

An ongoing need exists in the art for effective treatments for disease, especially hyperplasias and cancers. However, non-specific effects, and the inability to target and modulate certain classes of proteins altogether, such as transcription factors, remain as obstacles to the development of effective anti-cancer agents. As such, small molecule therapeutic agents that leverage E3 ligase mediated protein degradation to target cancer-associated proteins, such as one or more SWESNF-related matrix-associated actin-dependent regulator of chromatin subfamily A (“SMARCA”) and/or polybromo-1 (“PB1”) protein, hold promise as therapeutic agents. Accordingly, there remains a need to find compounds that are degraders of protein which is expressed from the SMARCA gene useful as therapeutic agents.

### SUMMARY

Disclosed are compounds and pharmaceutically acceptable salts thereof, pharmaceutical compositions comprising said compounds or pharmaceutically acceptable salts thereof, and methods for use of said compounds, pharmaceutically acceptable salts thereof, and pharmaceutical compositions thereof, which find utility as inducers of targeted ubiquitination of protein which is expressed from the SMARCA gene, which are then degraded and/or inhibited by the monovalent compounds as described herein. An advantage of the compounds provided herein is that a broad range of pharmacological activities are possible, consistent with the degradation/inhibition of protein which is expressed from the SMARCA gene. In addition, the disclosure provides methods of using an effective amount of the compounds as described herein for the treatment or amelioration of a disease condition, such as cancer, e.g., lung cancer, in a subject in need thereof.

In some embodiments, the disclosed compounds are represented by formula I:

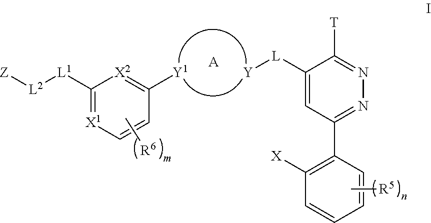

or a pharmaceutically acceptable salt, solvate, stereoisomer, or tautomer thereof, wherein L, L¹, L², m, n, R⁵, R⁶, T, X, Y, Y^{l}, X¹, X², Z, and Ring A are as defined herein.

In some embodiments, the disclosed compounds are represented by formula IA:

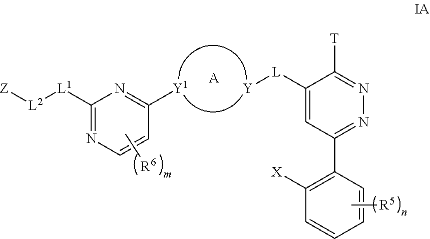

or a pharmaceutically acceptable salt, solvate, stereoisomer, or tautomer thereof, wherein L, L¹, L², m, n, R⁵, R⁶, T, X, Y, Y¹, Z, and Ring A are as defined herein.

In some embodiments, the disclosed compounds are represented by formula I′:

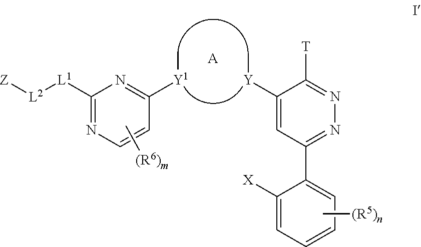

or a pharmaceutically acceptable salt, solvate, stereoisomer, or tautomer thereof, wherein L¹, L², m, n, R⁵, R⁶, T, X, Y, Y¹, Z, and Ring A are as defined herein.

In some embodiments, the compounds as described herein modulate protein which is expressed from the SMARCA gene. In some embodiments, the compounds as described herein degrade protein which is expressed from the SMARCA gene. In some embodiments, the protein which is modulated or degraded is expressed from the SMARCA gene member 2 (SMARCA2). In some embodiments, the protein which is modulated or degraded is expressed by the SMARCA gene member 4 (SMARCA4).

In some embodiments, this disclosure provides a pharmaceutical composition comprising a pharmaceutically acceptable excipient and an effective amount of a compound of formula I or I′ or any subformula thereof, or a pharmaceutically acceptable salt, solvate, stereoisomer, or tautomer thereof.

In some embodiments, this disclosure provides a method for modulating or degrading protein which is expressed from the SMARCA gene, which method comprises contacting the protein with an effective amount of a compound of formula I or I′ or any subformula thereof under conditions wherein the protein which is expressed from the SMARCA gene is bound to said compound and modulated or degraded. In some embodiments, the protein which is modulated or degraded is the protein which is expressed from the SMARCA2 gene. In some embodiments, the protein which is modulated or degraded is the protein which is expressed from the SMARCA4 gene.

In some embodiments, this disclosure provides a method for modulating or degrading protein which is expressed from the SMARCA gene in a subject, which method comprises administering to said subject an effective amount of a compound of formula I or I′ or any subformula thereof, or a pharmaceutical composition comprising a pharmaceutically acceptable excipient and an effective amount of a compound of formula I or I′ or any subformula thereof under conditions wherein the protein which is expressed from the SMARCA gene is bound to said compound and modulated or degraded. In some embodiments, the protein which is modulated or degraded in a subject is the protein which is expressed from the SMARCA2 gene. In some embodiments, the protein which is modulated or degraded in a subject is the protein which is expressed from the SMARCA4 gene.

In some embodiments, this disclosure provides a method for treating hyperplasias in a subject in need thereof, which method comprises administering to said subject an effective amount of a compound of formula I or I′ or any subformula thereof, or a pharmaceutical composition comprising a pharmaceutically acceptable excipient and an effective amount of a compound of formula I or I′ or any subformula thereof.

In some embodiments, this disclosure provides a method for treating cancer in a subject in need thereof, which method comprises administering to said subject an effective amount of a compound of formula I or I′ any subformula thereof, or a pharmaceutical composition comprising a pharmaceutically acceptable excipient and an effective amount of a compound of formula I or I′ or any subformula thereof.

### DETAILED DESCRIPTION

This disclosure provides for compounds, pharmaceutical compositions comprising such compounds, and methods of using such compounds and compositions to treat diseases, disorders, or conditions mediated, at least in part, by SMARCA2 or SMARCA4 transcription factors. However, prior to providing a detailed description of the disclosure, the following terms will first be defined. If not defined, terms used herein have their generally accepted scientific meaning.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise.

A dash (“-”) that is not between two letters or symbols is used to indicate a point of attachment for a substituent. For example, —C(O)NH₂ is attached through the carbon atom. A dash at the front or end of a chemical group is a matter of convenience; chemical groups may be depicted with or without one or more dashes without losing their ordinary meaning. A wavy line or a dashed line drawn through a line in a structure indicates a specified point of attachment of a group. Unless chemically or structurally required, no directionality or stereochemistry is indicated or implied by the order in which a chemical group is written or named.

The prefix “Cᵤ₋ᵥ” indicates that the following group has from u to v carbon atoms. For example, “C₁₋₆ alkyl” indicates that the alkyl group has from 1 to 6 carbon atoms.

The term “about” when used before a numerical designation, e.g., temperature, time, amount, concentration, and such other, including a range, indicates approximations which may vary by (+) or (−) 10%, 5%, 1%, or any subrange or subvalue there between. In one embodiment, the term “about” when used with regard to a dose amount means that the dose may vary by +/−10%.

“Comprising” or “comprises” is intended to mean that the compositions and methods include the recited elements, but not excluding others.

“Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed disclosure.

“Consisting of” shall mean excluding more than trace elements of other ingredients and substantial method steps. Embodiments defined by each of these transition terms are within the scope of this disclosure.

“Alkyl” refers to an unbranched or branched saturated hydrocarbon chain. As used herein, alkyl has 1 to 20 carbon atoms (i.e., C₁₋₂₀ alkyl), 1 to 12 carbon atoms (i.e., C₁₋₁₂ alkyl), 1 to 8 carbon atoms (i.e., C₁₋₈ alkyl), 1 to 6 carbon atoms (i.e., C₁₋₆ alkyl), or 1 to 4 carbon atoms (i.e., C₁₋₄ alkyl). Examples of alkyl groups include, e.g., methyl, ethyl, propyl, isopropyl, n-butyl, sec-butyl, iso-butyl, tert-butyl, pentyl, 2-pentyl, isopentyl, neopentyl, hexyl, 2-hexyl, 3-hexyl, and 3-methylpentyl. When an alkyl residue having a specific number of carbons is named by chemical name or identified by molecular formula, all positional isomers having that number of carbons may be encompassed; thus, for example, “butyl” includes n-butyl (i.e., —(CH₂)₃CH₃), sec-butyl (i.e., —CH(CH₃)CH₂CH₃), isobutyl (i.e., —CH₂CH(CH₃)₂), and tert-butyl (i.e., —C(CH₃)₃); and “propyl” includes n-propyl (i.e., —(CH₂)₂CH₃) and isopropyl (i.e., —CH(CH₃)₂).

Certain commonly used alternative chemical names may be used. For example, a divalent group such as a divalent “alkyl” group, a divalent “aryl” group, a divalent heteroaryl group, etc., may also be referred to as an “alkylene” group or an “alkylenyl” group (for example, methylenyl, ethylenyl, and propylenyl), an “arylene” group or an “arylenyl” group (for example, phenylenyl or napthylenyl, or quinolinyl for heteroarylene), respectively. Also, unless indicated explicitly otherwise, where combinations of groups are referred to herein as one moiety, e.g., arylalkyl or aralkyl, the last mentioned group contains the atom by which the moiety is attached to the rest of the molecule.

“Alkenyl” refers to an alkyl group containing at least one (e.g., 1-3, or 1) carbon-carbon double bond and having from 2 to 20 carbon atoms (i.e., C₂₋₂₀ alkenyl), 2 to 12 carbon atoms (i.e., C₂₋₁₂ alkenyl), 2 to 8 carbon atoms (i.e., C₂₋₈ alkenyl), 2 to 6 carbon atoms (i.e., C₂₋₆ alkenyl), or 2 to 4 carbon atoms (i.e., C₂₋₄ alkenyl). Examples of alkenyl groups include, e.g., ethenyl, propenyl, butadienyl (including 1,2-butadienyl and 1,3-butadienyl).

“Alkynyl” refers to an alkyl group containing at least one (e.g., 1-3, or 1) carbon-carbon triple bond and having from 2 to 20 carbon atoms (i.e., C₂₋₂₀ alkynyl), 2 to 12 carbon atoms (i.e., C₂₋₁₂ alkynyl), 2 to 8 carbon atoms (i.e., C₂₋₈ alkynyl), 2 to 6 carbon atoms (i.e., C₂₋₆ alkynyl), or 2 to 4 carbon atoms (i.e., C₂₋₄ alkynyl). The term “alkynyl” also includes those groups having one triple bond and one double bond.

“Alkoxy” refers to the group “alkyl-O—”. Examples of alkoxy groups include, e.g., methoxy, ethoxy, n-propoxy, iso-propoxy, n-butoxy, tert-butoxy, sec-butoxy, n-pentoxy, n-hexoxy, and 1,2-dimethylbutoxy.

“Alkylthio” refers to the group “alkyl-S—”. “Alkylsulfinyl” refers to the group “alkyl-S(O)—”. “Alkylsulfonyl” refers to the group “alkyl-S(O)₂—”. “Alkylsulfonylalkyl” refers to -alkyl-S(O)₂-alkyl.

“Acyl” refers to a group —C(O)R^{y}, wherein R^{y} is hydrogen, alkyl, alkenyl, alkynyl, cycloalkyl, heterocyclyl, aryl, heteroalkyl, or heteroaryl; each of which may be unsubstituted or substituted, as defined herein. Examples of acyl include, e.g., formyl, acetyl, cyclohexylcarbonyl, cyclohexylmethyl-carbonyl, and benzoyl.

“Amido” refers to both a “C-amido” group which refers to the group —C(O)NR^{y}R^{z} and an “N-amido” group which refers to the group —NR^{y}C(O)R^{z}, wherein R^{y} and R^{z} are independently hydrogen, alkyl, alkenyl, alkynyl, cycloalkyl, heterocyclyl, aryl, heteroalkyl, or heteroaryl; each of which may be unsubstituted or substituted, as defined herein, or R^{y} and R^{z} are taken together to form a cycloalkyl or heterocyclyl; each of which may be unsubstituted or substituted, as defined herein.

“Amino” refers to the group —NR^{y}R^{z} wherein R^{y} and R^{z} are independently hydrogen, alkyl, alkenyl, alkynyl, cycloalkyl, heterocyclyl, aryl, heteroalkyl, or heteroaryl; each of which may be unsubstituted or substituted, as defined herein.

“Amidino” refers to —C(NR^{y})(NR^{z}₂), wherein R^{y} and R^{z} are independently hydrogen, alkyl, alkenyl, alkynyl, cycloalkyl, heterocyclyl, aryl, heteroalkyl, or heteroaryl; each of which may be unsubstituted or substituted, as defined herein.

“Aryl” refers to an aromatic carbocyclic group having a single ring (e.g., monocyclic) or multiple rings (e.g., bicyclic or tricyclic) including fused systems. As used herein, aryl has 6 to 20 ring carbon atoms (i.e., C₆₋₂₀ aryl), 6 to 12 carbon ring atoms (i.e., C₆₋₁₂ aryl), or 6 to 10 carbon ring atoms (i.e., C₆₋₁₀ aryl). Examples of aryl groups include, e.g., phenyl, naphthyl, fluorenyl, and anthryl. Aryl, however, does not encompass or overlap in any way with heteroaryl defined below. If one or more aryl groups are fused with a heteroaryl, the resulting ring system is heteroaryl regardless of point of attachment. If one or more aryl groups are fused with a heterocyclyl, the resulting ring system is heterocyclyl regardless of point of attachment. If one or more aryl groups are fused with a cycloalkyl, the resulting ring system is cycloalkyl regardless of point of attachment.

“Carbamoyl” refers to both an “O-carbamoyl” group which refers to the group —O—C(O)NR^{y}R^{z} and an “N-carbamoyl” group which refers to the group —NR^{y}C(O)OR^{z}, wherein R^{y} and R^{z} are independently hydrogen, alkyl, alkenyl, alkynyl, cycloalkyl, heterocyclyl, aryl, heteroalkyl, or heteroaryl; each of which may be unsubstituted or substituted, as defined herein.

“Carboxyl ester” or “ester” refer to both —OC(O)R^{x} and —C(O)OR^{x}, wherein R^{x} is alkyl, alkenyl, alkynyl, cycloalkyl, heterocyclyl, aryl, heteroalkyl, or heteroaryl; each of which may be unsubstituted or substituted, as defined herein.

“Cycloalkyl” refers to a saturated or partially unsaturated cyclic alkyl group having a single ring or multiple rings including fused, bridged, and spiro ring systems. The term “cycloalkyl” includes cycloalkenyl groups (i.e., the cyclic group having at least one double bond) and carbocyclic fused ring systems having at least one sp³ carbon atom (i.e., at least one non-aromatic ring). As used herein, cycloalkyl has from 3 to 20 ring carbon atoms (i.e., C₃₋₂₀ cycloalkyl), 3 to 14 ring carbon atoms (i.e., C₃₋₁₄ cycloalkyl), 3 to 12 ring carbon atoms (i.e., C₃₋₁₂ cycloalkyl), 3 to 10 ring carbon atoms (i.e., C₃₋₁₀ cycloalkyl), 3 to 8 ring carbon atoms (i.e., C₃₋₈ cycloalkyl), or 3 to 6 ring carbon atoms (i.e., C₃₋₆ cycloalkyl). Monocyclic groups include, for example, cyclopropyl, cyclobutyl, cyclopentyl, cyclohexyl, cycloheptyl, and cyclooctyl. Polycyclic groups include, for example, bicyclo[2.2.1]heptanyl, bicyclo[2.2.2]octanyl, adamantyl, norbornyl, decalinyl, 7,7-dimethyl-bicyclo[2.2.1]heptanyl, and the like. Further, the term cycloalkyl is intended to encompass any non-aromatic ring which may be fused to an aryl ring, regardless of the attachment to the remainder of the molecule. Still further, cycloalkyl also includes “spirocycloalkyl” when there are two positions for substitution on the same carbon atom, for example spiro[2.5]octanyl, spiro[4.5]decanyl, or spiro[5.5]undecanyl.

“Imino” refers to a group —C(NR^{y})R^{z}, wherein R^{y} and R^{z} are each independently hydrogen, alkyl, alkenyl, alkynyl, cycloalkyl, heterocyclyl, aryl, heteroalkyl, or heteroaryl; each of which may be unsubstituted or substituted, as defined herein.

“Imido” refers to a group —C(O)NR^{y}C(O)R^{z}, wherein R^{y} and R^{z} are each independently hydrogen, alkyl, alkenyl, alkynyl, cycloalkyl, heterocyclyl, aryl, heteroalkyl, or heteroaryl; each of which may be unsubstituted or substituted, as defined herein.

“Halogen” or “halo” refers to atoms occupying group VIIA of the periodic table, such as fluoro, chloro, bromo, or iodo.

“Haloalkyl” refers to an unbranched or branched alkyl group as defined above, wherein one or more (e.g., 1 to 6 or 1 to 3) hydrogen atoms are replaced by a halogen. For example, where a residue is substituted with more than one halogen, it may be referred to by using a prefix corresponding to the number of halogen moieties attached. Dihaloalkyl and trihaloalkyl refer to alkyl substituted with two (“di”) or three (“tri”) halo groups, which may be, but are not necessarily, the same halogen. Examples of haloalkyl include, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like.

“Haloalkoxy” refers to an alkoxy group as defined above, wherein one or more (e.g., 1 to 6 or 1 to 3) hydrogen atoms are replaced by a halogen.

“Hydroxyalkyl” refers to an alkyl group as defined above, wherein one or more (e.g., 1 to 6 or 1 to 3) hydrogen atoms are replaced by a hydroxy group.

“Heteroalkyl” refers to an alkyl group in which one or more of the carbon atoms (and any associated hydrogen atoms), excluding any terminal carbon atom(s), are each independently replaced with the same or different heteroatomic group, provided the point of attachment to the remainder of the molecule is through a carbon atom. The term “heteroalkyl” includes unbranched or branched saturated chain having carbon and heteroatoms. By way of example, 1, 2 or 3 carbon atoms may be independently replaced with the same or different heteroatomic group. Heteroatomic groups include, but are not limited to, —NR^{y}—, —O—, —S—, —S(O)—, —S(O)₂—, and the like, wherein R^{y} is hydrogen, alkyl, alkenyl, alkynyl, cycloalkyl, heterocyclyl, aryl, heteroalkyl, or heteroaryl; each of which may be unsubstituted or substituted, as defined herein. Examples of heteroalkyl groups include, e.g., ethers (e.g., —CH₂OCH₃, —CH(CH₃)OCH₃, —CH₂CH₂OCH₃, —CH₂CH₂OCH₂CH₂OCH₃, etc.), thioethers (e.g., —CH₂SCH₃, —CH(CH₃)SCH₃, —CH₂CH₂SCH₃, —CH₂CH₂SCH₂CH₂SCH₃, etc.), sulfones (e.g., —CH₂S(O)₂CH₃, —CH(CH₃)S(O)₂CH₃, —CH₂CH₂S(O)₂CH₃, —CH₂CH₂S(O)₂CH₂CH₂OCH₃, etc.), and amines (e.g., —CH₂NR^{y}CH₃, —CH(CH₃)NR^{y}CH₃, —CH₂CH₂NR^{y}CH₃, —CH₂CH₂NR^{y}CH₂CH₂NR^{y}CH₃, etc., where R^{y} is hydrogen, alkyl, alkenyl, alkynyl, cycloalkyl, heterocyclyl, aryl, heteroalkyl, or heteroaryl; each of which may be unsubstituted or substituted, as defined herein). As used herein, heteroalkyl includes 2 to 10 carbon atoms, 2 to 8 carbon atoms, or 2 to 4 carbon atoms; and 1 to 3 heteroatoms, 1 to 2 heteroatoms, or 1 heteroatom.

“Heteroaryl” refers to an aromatic group having a single ring, multiple rings or multiple fused rings, with one or more ring heteroatoms independently selected from nitrogen, oxygen, and sulfur. As used herein, heteroaryl includes 1 to 20 ring carbon atoms (i.e., C₁₋₂₀ heteroaryl), 3 to 12 ring carbon atoms (i.e., C₃₋₁₂ heteroaryl), or 3 to 8 carbon ring atoms (i.e., C₃₋₈ heteroaryl), and 1 to 5 ring heteroatoms, 1 to 4 ring heteroatoms, 1 to 3 ring heteroatoms, 1 to 2 ring heteroatoms, or 1 ring heteroatom independently selected from nitrogen, oxygen, and sulfur. In certain instances, heteroaryl includes 5-10 membered ring systems, 5-7 membered ring systems, or 5-6 membered ring systems, each independently having 1 to 4 ring heteroatoms, 1 to 3 ring heteroatoms, 1 to 2 ring heteroatoms, or 1 ring heteroatom independently selected from nitrogen, oxygen, and sulfur. Examples of heteroaryl groups include, e.g., acridinyl, benzimidazolyl, benzothiazolyl, benzindolyl, benzofuranyl, benzothiazolyl, benzothiadiazolyl, benzonaphthofuranyl, benzoxazolyl, benzothienyl (benzothiophenyl), benzotriazolyl, benzo[4,6]imidazo[1,2-a]pyridyl, carbazolyl, cinnolinyl, dibenzofuranyl, dibenzothiophenyl, furanyl, isothiazolyl, imidazolyl, indazolyl, indolyl, indazolyl, isoindolyl, isoquinolyl, isoxazolyl, naphthyridinyl, oxadiazolyl, oxazolyl, 1-oxidopyridinyl, 1-oxidopyrimidinyl, 1-oxidopyrazinyl, 1-oxidopyridazinyl, phenazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, thiazolyl, thiadiazolyl, thiophenyl (i.e., thienyl), triazolyl, tetrazolyl, and triazinyl. Examples of the fused-heteroaryl rings include, but are not limited to, benzo[d]thiazolyl, quinolinyl, isoquinolinyl, benzo[b]thiophenyl, indazolyl, benzo[d]imidazolyl, pyrazolo[1,5-a]pyridinyl, and imidazo[1,5-a]pyridinyl, where the heteroaryl can be bound via either ring of the fused system. Any aromatic ring, having a single or multiple fused rings, containing at least one heteroatom, is considered a heteroaryl regardless of the attachment to the remainder of the molecule (i.e., through any one of the fused rings). Heteroaryl does not encompass or overlap with aryl as defined above.

“Heterocyclyl”—used interchangeably with “heterocycloalkyl”—refers to a saturated or partially unsaturated cyclic alkyl group, with one or more ring heteroatoms independently selected from nitrogen, oxygen, and sulfur. The term “heterocyclyl” includes heterocycloalkenyl groups (i.e., the heterocyclyl group having at least one double bond), bridged-heterocyclyl groups, fused-heterocyclyl groups, and spiro-heterocyclyl groups. A heterocyclyl may be a single ring or multiple rings wherein the multiple rings may be fused, bridged, or spiro, and may comprise one or more (e.g., 1 to 3) oxo (=O) or N-oxide (—O—) moieties. Any non-aromatic ring containing at least one heteroatom is considered a heterocyclyl, regardless of the attachment (i.e., can be bound through a carbon atom or a heteroatom). Further, the term heterocyclyl is intended to encompass any non-aromatic ring containing at least one heteroatom, which ring may be fused to a cycloalkyl, an aryl, or heteroaryl ring, regardless of the attachment to the remainder of the molecule. As used herein, heterocyclyl has 2 to 20 ring carbon atoms (i.e., C₂₋₂₀ heterocyclyl), 2 to 12 ring carbon atoms (i.e., C₂₋₁₂ heterocyclyl), 2 to 10 ring carbon atoms (i.e., C₂₋₁₀ heterocyclyl), 2 to 8 ring carbon atoms (i.e., C₂₋₈ heterocyclyl), 3 to 12 ring carbon atoms (i.e., C₃₋₁₂ heterocyclyl), 3 to 8 ring carbon atoms (i.e., C₃₋₈ heterocyclyl), or 3 to 6 ring carbon atoms (i.e., C₃₋₆ heterocyclyl); having 1 to 5 ring heteroatoms, 1 to 4 ring heteroatoms, 1 to 3 ring heteroatoms, 1 to 2 ring heteroatoms, or 1 ring heteroatom independently selected from nitrogen, sulfur, or oxygen. Examples of heterocyclyl groups include, e.g., azetidinyl, azepinyl, benzodioxolyl, benzo[b][1,4]dioxepinyl, 1,4-benzodioxanyl, benzopyranyl, benzodioxinyl, benzopyranonyl, benzofuranonyl, dioxolanyl, dihydropyranyl, hydropyranyl, thienyl[1,3]dithianyl, decahydroisoquinolyl, furanonyl, imidazolinyl, imidazolidinyl, indolinyl, indolizinyl, isoindolinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, oxiranyl, oxetanyl, phenothiazinyl, phenoxazinyl, piperidinyl, piperazinyl, 4-piperidonyl, pyrrolidinyl, pyrazolidinyl, quinuclidinyl, thiazolidinyl, tetrahydrofuryl, tetrahydropyranyl, trithianyl, tetrahydroquinolinyl, thiomorpholinyl, thiamorpholinyl, 1-oxo-thiomorpholinyl, and 1,1-dioxo-thiomorpholinyl. The term “heterocyclyl” also includes “spiroheterocyclyl” when there are two positions for substitution on the same carbon atom. Examples of the spiro-heterocyclyl rings include, e.g., bicyclic and tricyclic ring systems, such as oxabicyclo[2.2.2]octanyl, 2-oxa-7-azaspiro[3.5]nonanyl, 2-oxa-6-azaspiro[3.4]octanyl, and 6-oxa-1-azaspiro[3.3]heptanyl. Examples of the fused-heterocyclyl rings include, but are not limited to, 1,2,3,4-tetrahydroisoquinolinyl, 4,5,6,7-tetrahydrothieno[2,3-c]pyridinyl, indolinyl, and isoindolinyl, where the heterocyclyl can be bound via either ring of the fused system. In some embodiments, the heterocycloalkyl may be substituted with oxo group(s) on a heteroatom (e.g., S═O, S(═O)₂).

“Oxime” refers to the group —CR^{y}(=NOH) wherein R^{y} is hydrogen, alkyl, alkenyl, alkynyl, cycloalkyl, heterocyclyl, aryl, heteroalkyl, or heteroaryl; each of which may be unsubstituted or substituted, as defined herein.

“Oxo” refers to the moiety ═O.

“Sulfonyl” refers to the group —S(O)₂R^{y}, where R^{y} is hydrogen, alkyl, alkenyl, alkynyl, cycloalkyl, heterocyclyl, aryl, heteroalkyl, or heteroaryl; each of which may be unsubstituted or substituted, as defined herein. Examples of sulfonyl are methylsulfonyl, ethylsulfonyl, phenylsulfonyl, and toluenesulfonyl.

“Sulfinyl” refers to the group —S(O)R^{y}, where R^{y} is hydrogen, alkyl, alkenyl, alkynyl, cycloalkyl, heterocyclyl, aryl, heteroalkyl, or heteroaryl; each of which may be unsubstituted or substituted, as defined herein. Examples of sulfinyl are methylsulfinyl, ethylsulfinyl, phenylsulfinyl, and toluenesulfinyl.

“Sulfonamido” refers to the groups —SO₂NR^{y}R^{z} and —NR^{y}SO₂R^{z}, where R^{y} and R^{z} are each independently hydrogen, alkyl, alkenyl, alkynyl, cycloalkyl, heterocyclyl, aryl, heteroalkyl, or heteroaryl; each of which may be unsubstituted or substituted, as defined herein.

The terms “optional” or “optionally” means that the subsequently described event or circumstance may or may not occur and that the description includes instances where said event or circumstance occurs and instances in which it does not. Also, the term “unsubstituted or substituted” refers to any one or more (e.g., 1 to 5 or 1 to 3) hydrogen atoms on the designated atom or group may or may not be replaced by a moiety other than hydrogen.

The term “substituted” used herein means any of the above groups (i.e., alkyl, alkenyl, alkynyl, alkylene, alkoxy, haloalkyl, haloalkoxy, cycloalkyl, aryl, heterocyclyl, heteroaryl, and/or heteroalkyl) wherein at least one (e.g., 1 to 5 or 1 to 3) hydrogen atom is replaced by a bond to a non-hydrogen atom such as, but not limited to alkyl, alkenyl, alkynyl, alkoxy, alkylthio, acyl, amido, amino, amidino, aryl, aralkyl, azido, carbamoyl, carboxyl, carboxyl ester, cyano, cycloalkyl, cycloalkylalkyl, guanadino, halo, haloalkyl, haloalkoxy, hydroxyalkyl, heteroalkyl, heteroaryl, heteroarylalkyl, heterocyclyl, heterocyclylalkyl, —NHNH₂, =NNH₂, imino, imido, hydroxy, oxo, oxime, nitro, sulfonyl, sulfinyl, alkylsulfonyl, alkylsulfinyl, thiocyanate, —S(O)OH, —S(O)₂OH, sulfonamido, thiol, thioxo, N-oxide, or —Si(R^{y})₃, wherein each R^{y} is independently hydrogen, alkyl, alkenyl, alkynyl, heteroalkyl, cycloalkyl, aryl, heteroaryl, or heterocyclyl.

In certain embodiments, “substituted” includes any of the above alkyl, alkenyl, alkynyl, cycloalkyl, heterocyclyl, aryl, or heteroaryl groups in which one or more (e.g., 1 to 5 or 1 to 3) hydrogen atoms are independently replaced with deuterium, halo, cyano, nitro, azido, oxo, alkyl, alkenyl, alkynyl, haloalkyl, cycloalkyl, heterocyclyl, aryl, heteroaryl, —NR^{g}R^{h}, —NR^{g}C(O)R^{h}, —NR^{g}C(O)NR^{g}R^{h}, —NR^{g}C(O)OR^{h}, —NR^{g}S(O)₁₋₂R^{h}, —C(O)R^{g}, —C(O)OR^{g}, —OC(O)OR^{g}, —OC(O)R^{g}, —C(O)NR^{g}R^{h}, —OC(O)NR^{g}R^{h}, —OR^{g}, —SR^{g}, —S(O)R^{g}, —S(O)₂R^{g}, —OS(O)₁₋₂R^{g}, —S(O)₁₋₂OR^{g}, —NR^{g}S(O)₁₋₂NR^{g}R^{h}, =NSO₂R^{g}, =NOR^{g}, —S(O)₁₋₂NR^{g}R^{h}, —SF₅, —SCF₃, or —OCF₃. In certain embodiments, “substituted” also means any of the above groups in which one or more (e.g., 1 to 5 or 1 to 3) hydrogen atoms are replaced with —C(O)R^{g}, —C(O)OR^{g}, —C(O)NR^{g}R^{h}, —CH₂SO₂R^{g}, or —CH₂SO₂NR^{g}R^{h}. In the foregoing, R^{g} and R^{h} are the same or different and independently hydrogen, alkyl, alkenyl, alkynyl, alkoxy, thioalkyl, aryl, aralkyl, cycloalkyl, cycloalkylalkyl, haloalkyl, heterocyclyl, heterocyclylalkyl, heteroaryl, and/or heteroarylalkyl. In certain embodiments, “substituted” also means any of the above groups in which one or more (e.g., 1 to 5 or 1 to 3) hydrogen atoms are replaced by a bond to an amino, cyano, hydroxy, imino, nitro, oxo, thioxo, halo, alkyl, alkoxy, alkylamino, thioalkyl, aryl, aralkyl, cycloalkyl, cycloalkylalkyl, haloalkyl, heterocyclyl, N-heterocyclyl, heterocyclylalkyl, heteroaryl, and/or heteroarylalkyl, or two of R^{g} and R^{h} are taken together with the atoms to which they are attached to form a heterocyclyl ring unsubstituted or substituted with oxo, halo, or alkyl unsubstituted or substituted with oxo, halo, amino, hydroxy, or alkoxy.

Polymers or similar indefinite structures arrived at by defining substituents with further substituents appended ad infinitum (e.g., a substituted aryl having a substituted alkyl which is itself substituted with a substituted aryl group, which is further substituted by a substituted heteroalkyl group, etc.) are not intended for inclusion herein. Unless otherwise noted, the maximum number of serial substitutions in compounds described herein is three. For example, serial substitutions of substituted aryl groups with two other substituted aryl groups are limited to ((substituted aryl)substituted aryl) substituted aryl. Similarly, the above definitions are not intended to include impermissible substitution patterns (e.g., methyl substituted with 5 fluorine atoms or heteroaryl groups having two adjacent oxygen ring atoms). Such impermissible substitution patterns are well known to the skilled artisan. When used to modify a chemical group, the term “substituted” may describe other chemical groups defined herein.

In certain embodiments, as used herein, the phrase “one or more” refers to one to five. In certain embodiments, as used herein, the phrase “one or more” refers to one to three.

Any compound or structure given herein, is also intended to represent unlabeled forms as well as isotopically labeled forms of the compounds. These forms of compounds may also be referred to as “isotopically enriched analogs.” Isotopically labeled compounds have structures depicted herein, except that one or more atoms are replaced by an atom having a selected atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Various isotopically labeled compounds of the present disclosure, for example those into which radioactive isotopes such as ³H and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The term “isotopically enriched analogs” includes “deuterated analogs” of compounds described herein in which one or more hydrogens is/are replaced by deuterium, such as a hydrogen on a carbon atom. Such compounds exhibit increased resistance to metabolism and are thus useful for increasing the half-life of any compound when administered to a mammal, particularly a human. See, for example, Foster, “Deuterium Isotope Effects in Studies of Drug Metabolism,” Trends Pharmacol. Sci. 5 (12): 524-527 (1984). Such compounds are synthesized by means well known in the art, for example by employing starting materials in which one or more hydrogens have been replaced by deuterium.

Deuterium labelled or substituted therapeutic compounds of the disclosure may have improved DMPK (drug metabolism and pharmacokinetics) properties, relating to absorption, distribution, metabolism, and excretion (ADME). Substitution with heavier isotopes such as deuterium may afford certain therapeutic advantages resulting from greater metabolic stability, for example increased in vivo half-life, reduced dosage requirements, and/or an improvement in therapeutic index. An ¹⁸F, ³H, or ¹¹C labeled compound may be useful for PET or SPECT or other imaging studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in a compound described herein.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen,” the position is understood to have hydrogen at its natural abundance isotopic composition. Accordingly, in the compounds of this disclosure any atom specifically designated as a deuterium (D) is meant to represent deuterium.

In many cases, the compounds of this disclosure are capable of forming acid and/or base salts by virtue of the presence of amino, and/or carboxyl groups, or groups similar thereto.

Provided are also or a pharmaceutically acceptable salt, isotopically enriched analog, deuterated analog, stereoisomer, mixture of stereoisomers, and prodrugs of the compounds described herein. “Pharmaceutically acceptable” or “physiologically acceptable” refer to compounds, salts, compositions, dosage forms, and other materials which are useful in preparing a pharmaceutical composition that is suitable for veterinary or human pharmaceutical use.

The term “pharmaceutically acceptable salt” of a given compound refers to salts that retain the biological effectiveness and properties of the given compound and which are not biologically or otherwise undesirable. “Pharmaceutically acceptable salts” or “physiologically acceptable salts” include, for example, salts with inorganic acids, and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic or organic acids. Salts derived from inorganic acids include, e.g., hydrochloric acid, hydrobromic acid, sulfuric acid, nitric acid, phosphoric acid, and the like. Salts derived from organic acids include, e.g., acetic acid, propionic acid, gluconic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. Likewise, pharmaceutically acceptable base addition salts can be prepared from inorganic or organic bases. Salts derived from inorganic bases include, by way of example only, sodium, potassium, lithium, aluminum, ammonium, calcium, and magnesium salts. Salts derived from organic bases include, but are not limited to, salts of primary, secondary, and tertiary amines, such as alkyl amines (i.e., NH₂(alkyl)), dialkyl amines (i.e., HN(alkyl)₂), trialkyl amines (i.e., N(alkyl)₃), substituted alkyl amines (i.e., NH₂(substituted alkyl)), di(substituted alkyl) amines (i.e., HN(substituted alkyl)₂), tri(substituted alkyl) amines (i.e., N(substituted alkyl)₃), alkenyl amines (i.e., NH₂(alkenyl)), dialkenyl amines (i.e., HN(alkenyl)₂), trialkenyl amines (i.e., N(alkenyl)₃), substituted atkenyl amines (i.e., NH₂(substituted alkenyl)), di(substituted alkenyl) amines (i.e., HN(substituted alkenyl)₂), tri(substituted alkenyl) amines (i.e., N(substituted alkenyl)₃, mono-, di- or tri-cycloalkyl amines (i.e., NH₂(cycloalkyl), HN(cycloalkyl)₂, N(cycloalkyl)₃), mono-, di- or tri-arylamines (i.e., NH₂(aryl), HN(aryl)₂, N(aryl)₃), or mixed amines, etc. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine, and the like.

Some of the compounds exist as tautomers. Tautomers are in equilibrium with one another. For example, amide containing compounds may exist in equilibrium with imidic acid tautomers. Regardless of which tautomer is shown and regardless of the nature of the equilibrium among tautomers, the compounds are understood by one of ordinary skill in the art to comprise both amide and imidic acid tautomers. Thus, the amide containing compounds are understood to include their imidic acid tautomers. Likewise, the imidic acid containing compounds are understood to include their amide tautomers.

The compounds, or their pharmaceutically acceptable salts include an asymmetric center and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and/or fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers.

A “stereoisomer” refers to a compound made up of the same atoms bonded by the same bonds but having different three-dimensional structures, which are not interchangeable. The present disclosure contemplates various stereoisomers, or mixtures thereof, and includes “enantiomers,” which refers to two stereoisomers whose molecules are nonsuperimposable mirror images of one another.

“Diastereomers” are stereoisomers that have at least two asymmetric atoms, but which are not mirror-images of each other.

Relative centers of the compounds as depicted herein are indicated graphically using the “thick bond” style (bold or parallel lines) and absolute stereochemistry is depicted using wedge bonds (bold or parallel lines).

“Prodrug” means any compound which releases an active parent drug according to a structure described herein in vivo when such prodrug is administered to a mammalian subject. Prodrugs of a compound described herein are prepared by modifying functional groups present in the compound described herein in such a way that the modifications may be cleaved in vivo to release the parent compound. Prodrugs may be prepared by modifying functional groups present in the compounds in such a way that the modifications are cleaved, either in routine manipulation or in vivo, to the parent compounds. Prodrugs include compounds described herein wherein a hydroxy, amino, carboxyl, or sulfhydryl group in a compound described herein is bonded to any group that may be cleaved in vivo to regenerate the free hydroxy, amino, or sulfhydryl group, respectively. Examples of prodrugs include, but are not limited to esters (e.g., acetate, formate, and benzoate derivatives), amides, guanidines, carbamates (e.g., N,N-dimethylaminocarbonyl) of hydroxy functional groups in compounds described herein, and the like. Preparation, selection, and use of prodrugs is discussed in T. Higuchi and V. Stella, “Pro-drugs as Novel Delivery Systems,” Vol. 14 of the A.C.S. Symposium Series; “Design of Prodrugs,” ed. H. Bundgaard, Elsevier, 1985; and in Bioreversible Carriers in Drug Design, ed. Edward B. Roche, American Pharmaceutical Association and Pergamon Press, 1987, each of which are hereby incorporated by reference in their entirety.

“Subject” refers to a mammal. The mammal can be a human or non-human mammalian organism. A “patient” refers to a human subject.

“Treating” or “treatment” of a disease or disorder in a subject refers to 1) preventing the disease or disorder from occurring in a subject that is predisposed or does not yet display symptoms of the disease or disorder; 2) inhibiting the disease or disorder or arresting its development; or 3) ameliorating or causing regression of the disease or disorder.

“Effective amount” refers to the amount of a compound described herein that is sufficient to treat the disease or disorder afflicting a subject or to prevent such a disease or disorder from arising in said subject or patient.

“Administration” refers to any art recognized form of administration to a subject including oral (including oral gavage), pulmonary, transdermal, sublingual, injection (e.g., intravenous, intramuscular), transmucosal (e.g., vaginal, nasal, etc.), and the like. The route of administration is selected by the attending clinician and is based on factors such as the age, weight and general health of the patient as well as the severity of the condition. In one embodiment, the compounds and pharmaceutical compositions described herein are administered orally.

The term “ubiquitin ligase” refers to a family of proteins that facilitate the transfer of ubiquitin to a specific substrate protein, targeting the substrate protein for degradation. For example, an E3 ubiquitin ligase protein that alone or in combination with an E2 ubiquitin-conjugating enzyme causes the attachment of ubiquitin to a lysine on a target protein, and subsequently targets the specific protein substrates for degradation by the proteasome, Thus, E3 ubiquitin ligase alone or in complex with an E2 ubiquitin conjugating enzyme is responsible for the transfer of ubiquitin to targeted proteins, In general, the ubiquitin ligase is involved in polyubiquitination such that a second ubiquitin is attached to the first; a third is attached to the second, and so forth. Polyubiquitination marks proteins for degradation by the proteasome. However, there are some ubiquitination events that are limited to mono-ubiquitination, in which only a single ubiquitin is added by the ubiquitin ligase to a substrate molecule. Mono-ubiquitinated proteins are not targeted to the proteasome for degradation, but may instead he altered in their cellular location or function, for example, via binding other proteins that have domains capable of binding ubiquitin. Further complicating matters, different lysines on ubiquitin can be targeted by an E3 to make chains. The most common lysine is Lys48 on the ubiquitin chain. This is the lysine used to make polyubiquitin, which is recognized by the proteasome.

### Compounds

In one embodiment, this disclosure provides a compound of formula I:

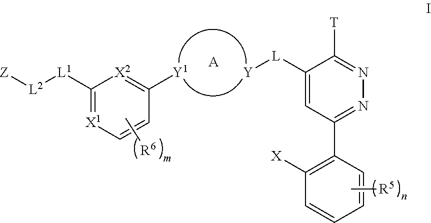

or a pharmaceutically acceptable salt, solvate, stereoisomer, or tautomer thereof, wherein:
X is hydroxy, halo, cyano, C₁₋₄ alkoxy, or —N(R)₂;
Y is N, C, or CH;
Y¹ is N, C, or CH;
X¹ is either CH, CR⁶, or N;
X² is either CH, CR⁶, or N; wherein at least one of X¹ or X² is N;
T is —N(R)₂;
Ring A is C₄₋₈ cycloalkyl, C₆₋₁₀ aryl, 4- to 11-membered heterocycloalkyl, or 5- to 10-membered heteroaryl, wherein each heterocycloalkyl or heteroaryl have from 1 to 3 heteroatoms selected from O, N, NR, and S, and wherein each cycloalkyl, aryl, heterocycloalkyl, or heteroaryl is independently unsubstituted or substituted with one to four R¹;
L is a bond, —CH₂—, or —O—;
L¹ is —RC═CR— or —C≡C—;
L² is selected from:

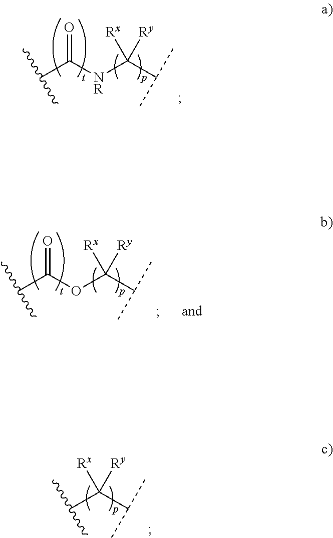

wherein t is 0 or 1, p is 0, 1, 2, or 3, and wherein the dashed line ( [symbol] ) denotes the point of attachment from L² to L¹, and the wavy line ( [symbol] ) denotes the point of attachment from L² to Z;
Z is C₁₋₁₀ alkyl, —N(R)₂, —OH, C₁₋₁₀ alkoxy, C₃₋₈ cycloalkyl, 4- to 10-membered heterocycloalkyl, or 5- to 10-membered heteroaryl, wherein each heterocycloalkyl or heteroaryl have from 1 to 4 heteroatoms selected from NR, N, O, and S, and wherein each heterocycloalkyl or heteroaryl is independently unsubstituted or substituted with one to four R², and wherein each alkyl, alkoxy, or cycloalkyl is independently unsubstituted or substituted with one to three R³;
each R is independently hydrogen, C₁₋₄ alkyl, or C₃₋₆ cycloalkyl, wherein each alkyl or cycloalkyl is unsubstituted or substituted with one to three R⁴; or
two R groups, when attached to the same nitrogen, form a 4- to 7-membered heterocycloalkyl, which is unsubstituted or substituted with one to four R⁴;
each R^{x} and R^{y} is independently hydrogen, C₁₋₄ alkyl, or C₁₋₄ haloalkyl substituted with 1 to 3 halo; or
R^{x} and R^{y}, together with the atoms to which they are attached, form a C₃₋₆ cycloalkyl, which is unsubstituted or substituted with one to four R⁴;
each R¹ is independently cyano, halo, hydroxyl, nitro, oxo, —N(R)₂, C₁₋₄ alkyl, C₁₋₄ haloalkyl substituted with 1 to 3 halo, C₁₋₄ alkoxy unsubstituted or substituted with 1 to 3 halo, C₅₋₆ heteroaryl having from 1 to 3 heteroatoms selected from O, N, NR, and/or S, 4- to 7-membered heterocycloalkyl having from 1 to 3 heteroatoms selected from oxygen, nitrogen, and/or sulfur, —C(O)OR, —OC(O)R, or —C(O)R;
each R² is independently oxo, hydroxy, cyano, halo, —N(R)₂, —C(O)OR, —NRC(O)OR, C₁₋₄ alkyl, C₁₋₄ haloalkyl substituted with from 1 to 3 halo groups, C₁₋₄ alkoxy, C₁₋₄ alkoxy substituted with from 1 to 3 halo groups, C₁₋₄ alkyl substituted with C₁₋₂ alkoxy, C₅₋₆ heteroaryl having from 1 to 3 heteroatoms selected from O, N, and NR, or C₃₋₆ cycloalkyl;
each R³ is independently oxo, cyano, hydroxy, halogen, C₁₋₄ alkyl, C₁₋₄ alkoxy, C₁₋₄ alkoxy substituted with from 1 to 3 halo groups, C₁₋₄ haloalkyl substituted with from 1 to 3 halo groups, C₁₋₆ aryl, C₁₋₄ alkyl substituted with 1 to 3 phenyl, or C₃₋₆ cycloalkyl;
each R⁴ is independently —N(R)₂, cyano, halo, or hydroxyl;
each R^{S} is D, halo, C₁₋₃ haloalkyl, or C₁₋₄ alkoxy;
each R⁶ is N(R)₂, SR, halo, cyano, C₁₋₄ alkoxy unsubstituted or substituted with 1 to 3 halo, unsubstituted C₁₋₄ alkyl, C₁₋₄ alkyl substituted with C₁₋₂ alkoxy, or C₁₋₄ alkyl substituted with hydroxy;
m is 0, 1, or 2; and
n is 0, 1, 2, or 3;
with the proviso that when the compound

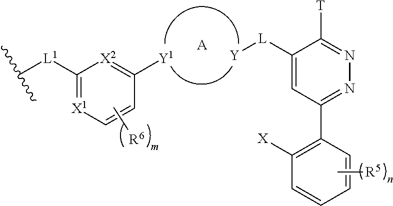

is the following moiety:

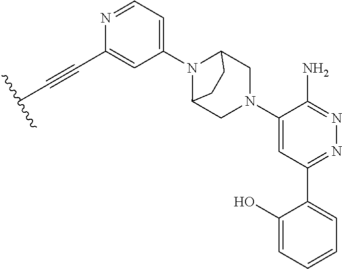 [SMILES: *C#Cc1cc(N2C3CCC2CN(c2cc(-c4ccccc4O)nnc2N)C3)ccn1]

and L² is a —C(O)NH—, bond, —CH₂—, or —(CH₂)₂—, then Z is not a heterocyclyl (wherein the heterocyclyl is unsubstituted or substituted), —C₁₋₆ alkylene-NH₂, or —C₁₋₆ alkylene-OH,
and the compound is not:
2-(6-amino-5-(8-(2-(3-((2-hydroxyethyl)(methyl)amino)prop-1-yn-1-yl)pyridin-4-yl)-3,8-diazabicyclo[3.2.1]octan-3-yl)pyridazin-3-yl)phenol,
2-(6-amino-5-(8-(2-(3-(2-hydroxyethoxyprop-1-yn-1-yl)pyridin-4-yl)-3,8-diazabicyclo[3.2.1]octan-3-yl)pyridazin-3-yl)phenol,
2-(6-amino-5-(8-(2-(3-(diethylamino)prop-1-yn-1-yl)pyridin-4-yl)-3,8-diazabicyclo[3.2.1]octan-3-yl)pyridazin-3-yl)phenol,
2-(6-amino-5-(8-(2-(3-(piperidin-3-yloxy)prop-1-yn-1-yl)pyridin-4-yl)-3,8-diazabicyclo[3.2.1]octan-3-yl)pyridazin-3-yl)phenol,
2-(6-amino-5-(8-(2-((1-methyl-1H-pyrazol-4-yl)ethynyl)pyridin-4-yl)-3,8-diazabicyclo[3.2.1]octan-3-yl)pyridazin-3-yl)phenol,
N-[3-[4-[3-[3-amino-6-(2-hydroxyphenyl)pyridazin-4-yl]-3,8-diazabicyclo[3.2.1]octan-8-yl]-2-pyridyl]prop-2-ynyl]piperidine-4-carboxamide,
2-[6-amino-5-[8-[2-[3-(azepan-1-yl)prop-1-ynyl]pyrimidin-4-yl]-3,8-diazabicyclo[3.2.1]octan-3-yl]pyridazin-3-yl]phenol,
2-[6-amino-5-[9-[2-[3-(azepan-1-yl)prop-1-ynyl]-4-pyridyl]-3,9-diazaspiro[5.5]undecan-3-yl]pyridazin-3-yl]phenol,
2-[6-amino-5-[2-[2-[3-(azepan-1-yl)prop-1-ynyl]-4-pyridyl]-2,8-diazaspiro[4.5]decan-8-yl]pyridazin-3-yl]phenol,
2-|6-amino-5-|6-|2-|3-(azepan-1-yl)prop-1-ynyl|-4-pyridyl|-2,6-diazaspiro|3.3|heptan-2-yl]pyridazin-3-yl]phenol,
2-(6-amino-5-(8-(2-(((1r,4r)-4-aminocyclohexyl)ethynyl)pyridin-4-yl)-3,8-diazabicyclo[3.2.1]octan-3-yl)pyridazin-3-yl)phenol, or
2-[6-amino-5-[8-[2-[(E)-3-(azepan-1-yl)prop1-enyl]-4-pyridyl]-3,8-diazabicyclo[3.2.1]octan-3-yl]pyridazin-3-yl]phenol.

In one embodiment, this disclosure provides a compound of formula IA:

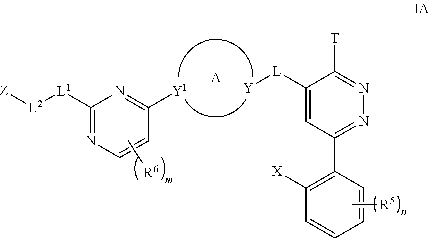

or a pharmaceutically acceptable salt, solvate, stereoisomer, or tautomer thereof, wherein:
X is hydroxy, halo, cyano, C₁₋₄ alkoxy, or —N(R)₂;
Y is N, C, or CH;
Y¹ is N, C, or CH;
T is —N(R)₂;
Ring A is C₄₋₈ cycloalkyl, C₆₋₁₀ aryl, 4- to 11-membered heterocycloalkyl, or 5- to 10-membered heteroaryl, wherein each heterocycloalkyl or heteroaryl have from 1 to 3 heteroatoms selected from O, N, NR, and S, and wherein each cycloalkyl, aryl, heterocycloalkyl, or heteroaryl is independently unsubstituted or substituted with one to four R¹;
L is a bond, —CH₂—, or —O—;
L¹ is —RC=CR— or —C≡C—;
L² is selected from:

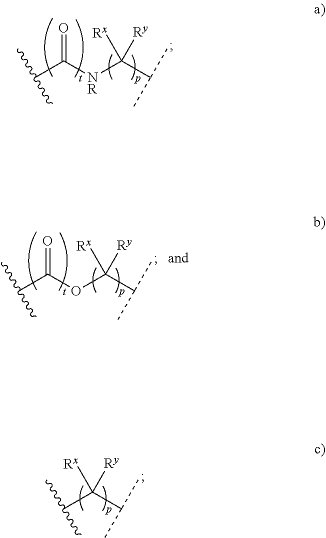

wherein t is 0 or 1, p is 0, 1, 2, or 3, and wherein the dashed line ( [symbol] ) denotes the point of attachment from L² to L¹, and the wavy line ( [symbol] ) denotes the point of attachment from L² to Z;
Z is C₁₋₁₀ alkyl, —N(R)₂, —OH, C₁₋₁₀ alkoxy, C₃₋₈ cycloalkyl, 4- to 10-membered heterocycloalkyl, or 5- to 10-membered heteroaryl, wherein each heterocycloalkyl or heteroaryl have from 1 to 4 heteroatoms selected from NR, N, O, and S, and wherein each heterocycloalkyl or heteroaryl is independently unsubstituted or substituted with one to four R², and wherein each alkyl, alkoxy, or cycloalkyl is independently unsubstituted or substituted with one to three R³;
each R is independently hydrogen, C₁₋₄ alkyl, or C₃₋₆ cycloalkyl, wherein each alkyl or cycloalkyl is unsubstituted or substituted with one to three R⁴; or
two R groups, when attached to the same nitrogen, form a 4- to 7-membered heterocycloalkyl, which is unsubstituted or substituted with one to four R⁴;
each R^{x} and R^{y} is independently hydrogen, C₁₋₄ alkyl, or C₁₋₄ haloalkyl substituted with 1 to 3 halo; or
R^{x} and R^{y}, together with the atoms to which they are attached, form a C₃₋₆ cycloalkyl, which is unsubstituted or substituted with one to four R⁴;
each R¹ is independently cyano, halo, hydroxyl, nitro, oxo, —N(R)₂, C₁₋₄ alkyl, C₁₋₄ haloalkyl substituted with 1 to 3 halo, C₁₋₄ alkoxy unsubstituted or substituted with 1 to 3 halo, C₅₋₆ heteroaryl having from 1 to 3 heteroatoms selected from O, N, NR, and/or S, 4- to 7-membered heterocycloalkyl having from 1 to 3 heteroatoms selected from oxygen, nitrogen, and/or sulfur, —C(O)OR, —OC(O)R, or —C(O)R;
each R² is independently oxo, hydroxy, cyano, halo, —N(R)₂, —C(O)OR, —NRC(O)OR, C₁₋₄ alkyl, C₁₋₄ haloalkyl substituted with from 1 to 3 halo groups, C₁₋₄ alkoxy, C₁₋₄ alkoxy substituted with from 1 to 3 halo groups, C₁₋₄ alkyl substituted with C₁₋₂ alkoxy, C₅₋₆ heteroaryl having from 1 to 3 heteroatoms selected from O, N, and NR, or C₃₋₆ cycloalkyl;
each R³ is independently oxo, cyano, hydroxy, halogen, C₁₋₄ alkyl, C₁₋₄ alkoxy, C₁₋₄ alkoxy substituted with from 1 to 3 halo groups, C₁₋₄ haloalkyl substituted with from 1 to 3 halo groups, C₁₋₆ aryl, C₁₋₄ alkyl substituted with 1 to 3 phenyl, or C₃₋₆ cycloalkyl;
each R⁴ is independently —N(R)₂, cyano, halo, or hydroxyl;
each R⁵ is D, halo, C₁₋₃ haloalkyl, or C₁₋₄ alkoxy;
each R⁶ is N(R)₂, SR, halo, cyano, C₁₋₄ alkoxy unsubstituted or substituted with 1 to 3 halo, unsubstituted C₁₋₄ alkyl, C₁₋₄ alkyl substituted with C₁₋₂ alkoxy, or C₁₋₄ alkyl substituted with hydroxy;
m is 0, 1, or 2; and
n is 0, 1, 2, or 3;
with the proviso that the compound is not 2-[6-amino-5-[8-[2-[3-(azepan-1-yl)prop-1-ynyl]pyrimidin-4-yl]-3,8-diazabicyclo[3.2.1]octan-3-yl]pyridazin-3-yl]phenol.

In one embodiment, this disclosure provides a compound of formula I′:

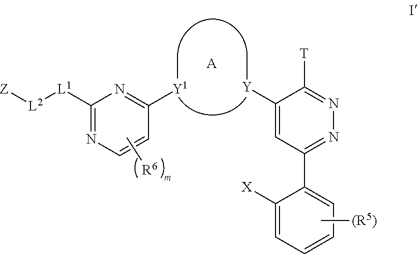

or a pharmaceutically acceptable salt, solvate, stereoisomer, or tautomer thereof, wherein:
X is hydroxy, halo, cyano, C₁₋₄ alkoxy, or —N(R)₂;
Y is N, C, or CH;
Y¹ is N, C, or CH;
T is —N(R)₂;
Ring A is C₄ s cycloalkyl, C₆₋₁₀ aryl, 4- to 11-membered heterocycloalkyl, or 5- to 10-membered heteroaryl, wherein each heterocycloalkyl or heteroaryl have from 1 to 3 heteroatoms selected from O, N, NR, and S, and wherein each cycloalkyl, aryl, heterocycloalkyl, or heteroaryl is independently unsubstituted or substituted with one to four R⁴;
L¹ is —RC=CR— or —C≡C—;
L² is selected from:

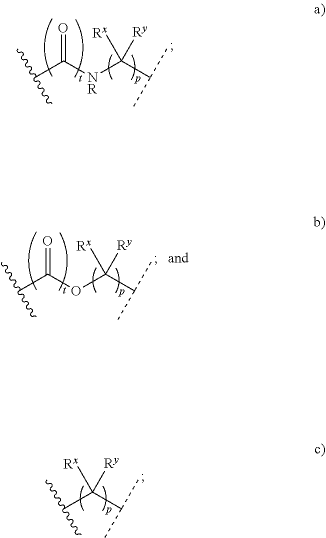

wherein t is 0 or 1, p is 0, 1, 2, or 3, and wherein the dashed line ( [symbol] ) denotes the point of attachment from L² to L¹, and the wavy line ( [symbol] ) denotes the point of attachment from L² to Z;
Z is C₁₋₁₀ alkyl, —N(R)₂, —OH, C₁₋₁₀ alkoxy, C₃₋₈ cycloalkyl, 4- to 10-membered heterocycloalkyl, or 5- to 10-membered heteroaryl, wherein each heterocycloalkyl or heteroaryl have from 1 to 4 heteroatoms selected from NR, N, O, and S, and wherein each heterocycloalkyl or heteroaryl is independently unsubstituted or substituted with one to four R², and wherein each alkyl, alkoxy, or cycloalkyl is independently unsubstituted or substituted with one to three R³;
each R is independently hydrogen, C₁₋₄ alkyl, or C₃₋₆ cycloalkyl, wherein each alkyl or cycloalkyl is unsubstituted or substituted with one to three R⁴; or
two R groups, when attached to the same nitrogen, form a 4- to 7-membered heterocycloalkyl, which is unsubstituted or substituted with one to four R⁴;
each R^{x} and R^{y} is independently hydrogen, C₁₋₄ alkyl, or C₁₋₄ haloalkyl substituted with 1 to 3 halo; or
R^{x} and R^{y}, together with the atoms to which they are attached, form a C₃₋₆ cycloalkyl, which is unsubstituted or substituted with one to four R⁴;
each R¹ is independently cyano, halo, hydroxyl, nitro, oxo, —N(R)₂, C₁₋₄ alkyl, C₁₋₄ haloalkyl substituted with 1 to 3 halo, C₁₋₄ alkoxy unsubstituted or substituted with 1 to 3 halo, C₅₋₆ heteroaryl having from 1 to 3 heteroatoms selected from O, N, NR, and/or S, 4- to 7-membered heterocycloalkyl having from 1 to 3 heteroatoms selected from oxygen, nitrogen, and/or sulfur, —C(O)OR, —OC(O)R, or —C(O)R;
each R² is independently oxo, hydroxy, cyano, halo, —N(R)₂, —C(O)OR, —NRC(O)OR, C₁₋₄ alkyl, C₁₋₄ haloalkyl substituted with from 1 to 3 halo groups, C₁₋₄ alkoxy, C₁₋₄ alkoxy substituted with from 1 to 3 halo groups, C₁₋₄ alkyl substituted with C₁₋₂ alkoxy, C₅₋₆ heteroaryl having from 1 to 3 heteroatoms selected from O, N, and NR, or C₃₋₆ cycloalkyl;
each R³ is independently oxo, halogen, C₁₋₄ alkyl, C₁₋₄ alkoxy, C₁₋₄ alkoxy substituted with from 1 to 3 halo groups, C₁₋₄ haloalkyl substituted with from 1 to 3 halo groups, or C₃₋₆ cycloalkyl;
each R⁴ is independently —N(R)₂, cyano, halo, or hydroxyl;
each R⁵ is D, halo, C₁₋₃ haloalkyl, or C₁₋₄ alkoxy;
each R⁶ is N(R)₂, SR, cyano, C₁₋₄ alkoxy unsubstituted or substituted with 1 to 3 halo, C₁₋₄ alkyl substituted with C₁₋₂ alkoxy, or C₁₋₄ alkyl substituted with hydroxy;
m is 0, 1, or 2; and
n is 0, 1, 2, or 3;
with the proviso that the compound is not 2-[6-amino-5-[8-[2-[3-(azepan-1-yl)prop-1-ynyl]pyrimidin-4-yl]-3,8-diazabicyclo[3.2.1]octan-3-yl]pyridazin-3-yl]phenol.

In one embodiment, this disclosure provides a compound of formula I′:

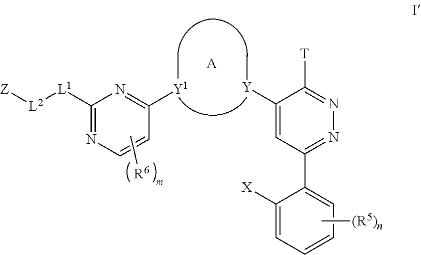

or a pharmaceutically acceptable salt, solvate, stereoisomer, or tautomer thereof, wherein:
X is hydroxy, halo, cyano, C₁₋₄ alkoxy, or —N(R)₂;
Y is N, C, or CH;
Y¹ is N, C, or CH;
T is —N(R)₂;
Ring A is C₄₋₈ cycloalkyl, C₆₋₁₀ aryl, 4- to 11-membered heterocycloalkyl, or 5- to 10-membered heteroaryl, wherein each heterocycloalkyl or heteroaryl have from 1 to 3 heteroatoms selected from O, N, NR, and S, and wherein each cycloalkyl, aryl, heterocycloalkyl, or heteroaryl is independently unsubstituted or substituted with one to four R¹;
L¹ is —RC=CR— or —C≡C—;
L² is selected from:

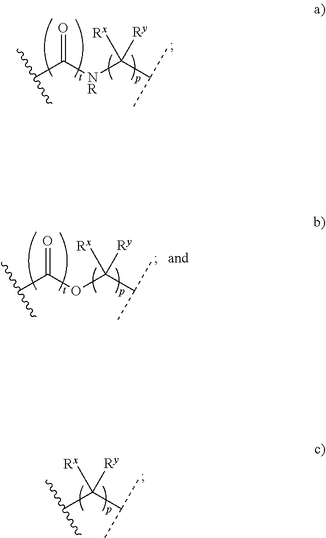

wherein t is 0 or 1, p is 0, 1, 2, or 3, and wherein the dashed line ( [symbol] ) denotes the point of attachment from L² to L¹, and the wavy line ( [symbol] ) denotes the point of attachment from L² to Z;
Z is C₁₋₁₀ alkyl, —N(R)₂, —OH, C₁₋₁₀ alkoxy, C₃₋₈ cycloalkyl, 4- to 10-membered heterocycloalkyl, or 5- to 10-membered heteroaryl, wherein each heterocycloalkyl or heteroaryl have from 1 to 4 heteroatoms selected from NR, N, O, and S, and wherein each heterocycloalkyl or heteroaryl is independently unsubstituted or substituted with one to four R², and wherein each alkyl, alkoxy, or cycloalkyl is independently unsubstituted or substituted with one to three R³;
each R is independently hydrogen, C₁₋₄ alkyl, or C₃₋₆ cycloalkyl, wherein each alkyl or cycloalkyl is unsubstituted or substituted with one to three R⁴; or
two R groups, when attached to the same nitrogen, form a 4- to 7-membered heterocycloalkyl, which is unsubstituted or substituted with one to four R⁴;
each R^{x} and R^{y} is independently hydrogen, C₁₋₄ alkyl, or C₁₋₄ haloalkyl substituted with 1 to 3 halo; or
R^{x} and R^{y}, together with the atoms to which they are attached, form a C₃₋₆ cycloalkyl, which is unsubstituted or substituted with one to four R⁴;
each R¹ is independently cyano, halo, hydroxyl, nitro, oxo, —N(R)₂, C₁₋₄ alkyl, C₁₋₄ haloalkyl substituted with 1 to 3 halo, C₁₋₄ alkoxy unsubstituted or substituted with 1 to 3 halo, C₅₋₆ heteroaryl having from 1 to 3 heteroatoms selected from O, N, NR, and/or S, 4- to 7-membered heterocycloalkyl having from 1 to 3 heteroatoms selected from oxygen, nitrogen, and/or sulfur, —C(O)OR, —OC(O)R, or —C(O)R;
each R² is independently oxo, hydroxy, cyano, halo, —N(R)₂, —C(O)OR, —NRC(O)OR, C₁₋₄ alkyl, C₁₋₄ haloalkyl substituted with from 1 to 3 halo groups, C₁₋₄ alkoxy, C₁₋₄ alkoxy substituted with from 1 to 3 halo groups, C₁₋₄ alkyl substituted with C₁₋₂ alkoxy, C₅₋₆ heteroaryl having from 1 to 3 heteroatoms selected from O, N, and NR, or C₃₋₆ cycloalkyl;
each R³ is independently oxo, halogen, C₁₋₄ alkyl, C₁₋₄ alkoxy, C₁₋₄ alkoxy substituted with from 1 to 3 halo groups, C₁₋₄ haloalkyl substituted with from 1 to 3 halo groups, or C₃₋₆ cycloalkyl;
each R⁴ is independently —N(R)₂, cyano, halo, or hydroxyl;
each R⁵ is D, halo, C₁₋₃ haloalkyl, or C₁₋₄ alkoxy;
each R⁶ is N(R)₂, SR, cyano, C₁₋₄ alkoxy unsubstituted or substituted with 1 to 3 halo, C₁₋₄ alkyl substituted with C₁₋₂ alkoxy, or C₁₋₄ alkyl substituted with hydroxy;
m is 0, 1, or 2; and
n is 0, 1, 2, or 3.

In some embodiments, provided is a compound of Formula 1 represented by Formula II:

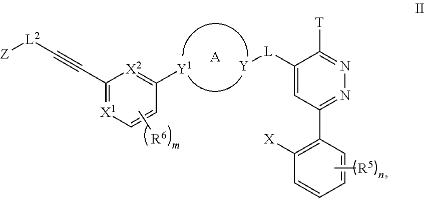

or a pharmaceutically acceptable salt, solvate, stereoisomer, or tautomer thereof,
wherein L, L², m, n, R⁵, R⁶, T, X, Y, Y¹. X¹, X², Z, and Ring A are each independently as defined herein.

In some embodiments, provided is a compound of Formula II represented by Formula II-1:

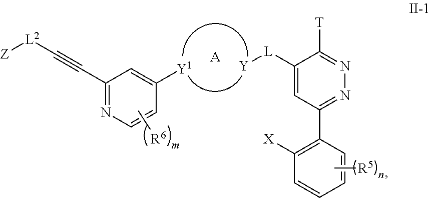

or a pharmaceutically acceptable salt, solvate, stereoisomer, or tautomer thereof,
wherein L, L², m, n, R⁵, R⁶, T, X, Y, Y¹, Z, and Ring A are each independently as defined herein.

In some embodiments, provided is a compound of Formula II represented by Formula II-2:

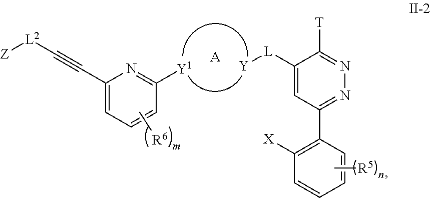

or a pharmaceutically acceptable salt, solvate, stereoisomer, or tautomer thereof,
wherein L, L², m, n, R⁵, R⁶, T, X, Y, Y¹, Z, and Ring A are each independently as defined herein.

In some embodiments, provided is a compound of Formula II represented by Formula II-3:

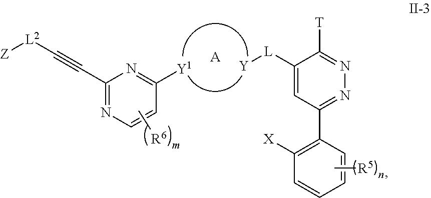

or a pharmaceutically acceptable salt, solvate, stereoisomer, or tautomer thereof,
wherein L, L², m, n, R⁵, R⁶, T, X, Y, Y¹, Z, and Ring A are each independently as defined herein.

In some embodiments, provided is a compound of Formula I represented by Formula IIa:

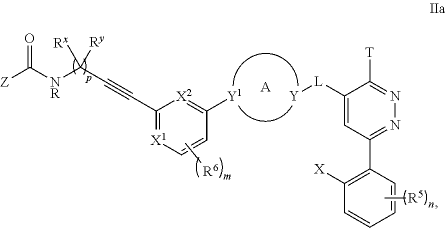

or a pharmaceutically acceptable salt, solvate, stereoisomer, or tautomer thereof,
wherein L, m, n, p, R, R^{x}, R^{y}, R⁵, R⁶, T, X, Y, Y¹, X¹, X², Z, and Ring A are each independently as defined herein.

In some embodiments, provided is a compound of Formula I represented by Formula IIb:

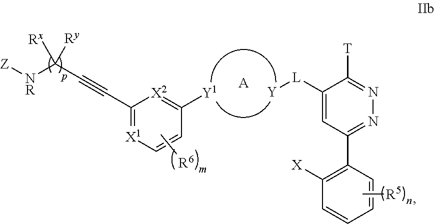

or a pharmaceutically acceptable salt, solvate, stereoisomer, or tautomer thereof,
wherein L, m, n, p, R, R^{x}, R^{y}, R⁵, R⁶, T, X, Y, Y¹, X¹, X², Z, and Ring A are each independently as defined herein.

In some embodiments, provided is a compound of Formula I represented by Formula IIc:

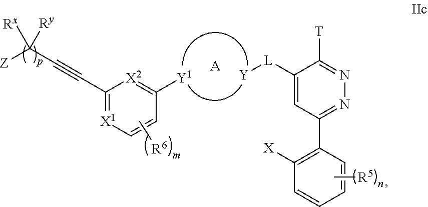

or a pharmaceutically acceptable salt, solvate, stereoisomer, or tautomer thereof,
wherein L, m, n, p, R^{x}, R^{y}, R⁵, R⁶, T, X, Y, Y¹, X¹, X², Z, and Ring A are each independently as defined herein.

In some embodiments, provided is a compound of Formula Ie represented by Formula IIc-1:

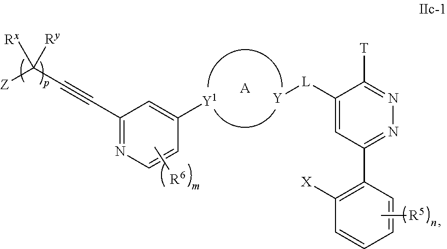

or a pharmaceutically acceptable salt, solvate, stereoisomer, or tautomer thereof,
wherein L, m, n, p, R^{x}, R^{y}, R⁵, R⁶, T, X, Y, Y¹, Z, and Ring A are each independently as defined herein.

In some embodiments, provided is a compound of Formula Ie represented by Formula IIc-2:

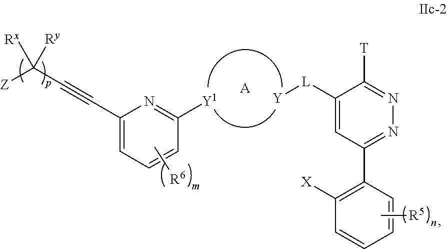

or a pharmaceutically acceptable salt, solvate, stereoisomer, or tautomer thereof,
wherein L, m, n, p, R^{x}, R^{y}, R⁵, R⁶, T, X, Y, Y¹, Z, and Ring A are each independently as defined herein.

In some embodiments, provided is a compound of Formula IIc represented by Formula IIc-3:

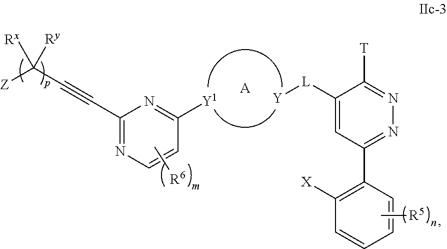

or a pharmaceutically acceptable salt, solvate, stereoisomer, or tautomer thereof,
wherein L, m, n, p, R^{x}, R^{y}, R⁵, R⁶, T, X, Y, Y¹, Z, and Ring A are each independently as defined herein.

In some embodiments, provided is a compound of Formula I represented by Formula III-E:

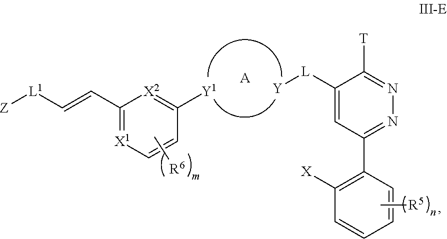

or a pharmaceutically acceptable salt, solvate, stereoisomer, or tautomer thereof,
wherein L, L¹, m, n, R⁵, R⁶, T, X, Y, Y¹, X¹, X², Z, and Ring A are each independently as defined herein.

In some embodiments, provided is a compound of Formula I represented by Formula IIIb-E:

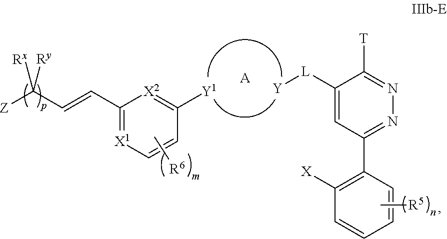

or a pharmaceutically acceptable salt, solvate, stereoisomer, or tautomer thereof,
wherein L, m, n, p, R^{x}, R^{y}, R⁵, R⁶, T, X, Y, Y¹, X¹, X², Z, and Ring A are each independently as defined herein.

In some embodiments, provided is a compound of Formula I′ represented by Formula II′:

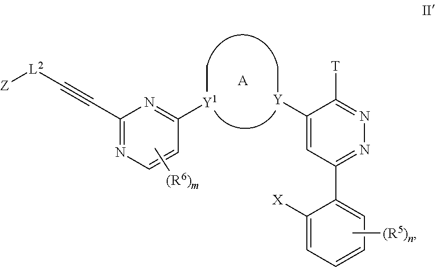

or a pharmaceutically acceptable salt, solvate, stereoisomer, or tautomer thereof,
wherein L², m, n, R⁵, R⁶, T, X, Y, Y¹, Z, and Ring A are each independently as defined herein.

In some embodiments, the compound of Formula II′ is represented by Formula IIa′:

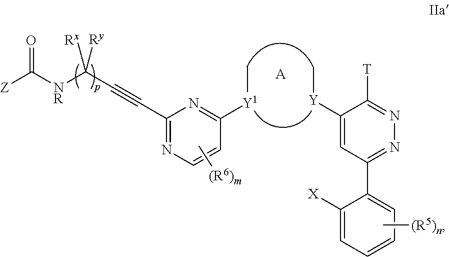

or a pharmaceutically acceptable salt, solvate, stereoisomer, or tautomer thereof,
wherein m, n, p, R, R⁵, R⁶, R^{x}, R^{y}, T, X, Y, Y¹, Z, and Ring A are each independently as defined herein.

In some embodiments, the compound of Formula IIa′ is represented by Formula IIa′-1:

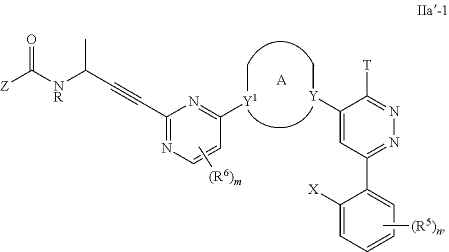

or a pharmaceutically acceptable salt, solvate, stereoisomer, or tautomer thereof,
wherein m, n, R, R⁵, R⁶, T, X, Y, Y¹, Z, and Ring A are each independently as defined herein.

In some embodiments, the compound of Formula IIa′ is represented by Formula IIa′-2:

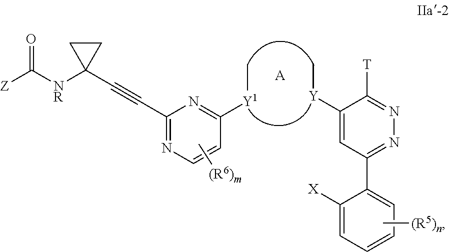

or a pharmaceutically acceptable salt, solvate, stereoisomer, or tautomer thereof,
wherein m, n, R, R⁵, R⁶, T, X, Y, Y¹, Z, and Ring A are each independently as defined herein.

In some embodiments, the compound of Formula II′ is represented by Formula IIb′:

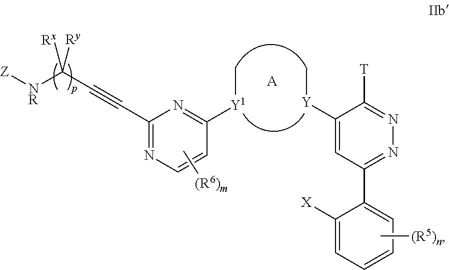

or a pharmaceutically acceptable salt, solvate, stereoisomer, or tautomer thereof,
wherein m, n, p, R, R⁵, R⁶, R^{x}, R^{y}, T, X, Y, Y¹, Z, and Ring A are each independently as defined herein.

In some embodiments, the compound of Formula II′ is represented by Formula IIc′:

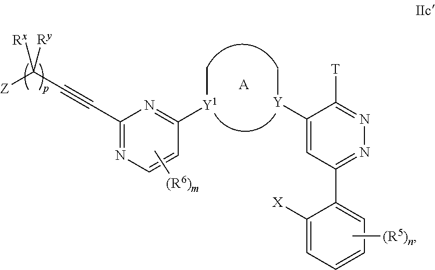

or a pharmaceutically acceptable salt, solvate, stereoisomer, or tautomer thereof,
wherein m, n, p, R⁵, R⁶, R^{x}, R^{y}, T, X, Y, Y¹, Z, and Ring A are each independently as defined herein.

In some embodiments, the compound of Formula II′ is represented by Formula IId′:

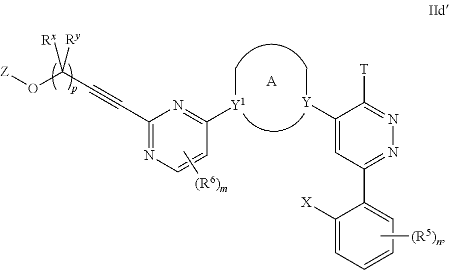

or a pharmaceutically acceptable salt, solvate, stereoisomer, or tautomer thereof,
wherein m, n, p, R⁵, R⁶, R^{x}, R^{y}, T, X, Y, Y¹, Z, and Ring A are each independently as defined herein.

In some embodiments, provided is a compound of Formula I′ represented by Formula III′-E:

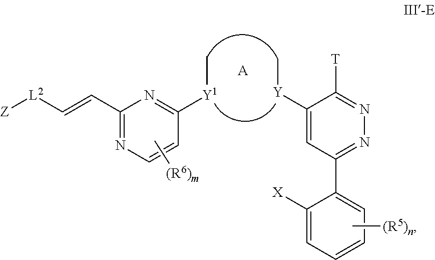

or a pharmaceutically acceptable salt, solvate, stereoisomer, or tautomer thereof,
wherein L², m, n, R⁵, R⁶, T, X, Y, Y¹, Z, and Ring A are each independently as defined herein.

In some embodiments, the compound of Formula III′-E is represented by Formula IIIa′-E:

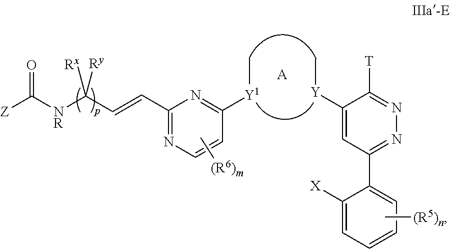

or a pharmaceutically acceptable salt, solvate, stereoisomer, or tautomer thereof,
wherein m, n, p, R, R⁵, R⁶, R^{x}, R^{y}, T, X, Y, Y¹, Z, and Ring A are each independently as defined herein.

In some embodiments, the compound of Formula III′-E is represented by Formula IIIb′-E:

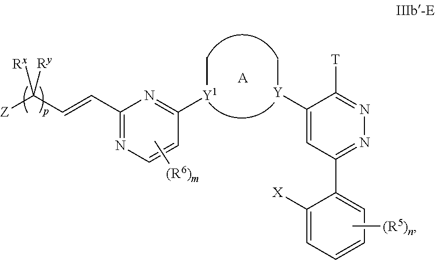

or a pharmaceutically acceptable salt, solvate, stereoisomer, or tautomer thereof,
wherein m, n, p, R⁵, R⁶, R^{x}, R^{y}, T, X, Y, Y¹, Z, and Ring A are each independently as defined herein.

In some embodiments, provided is a compound of Formula I′ represented by Formula III′-Z:

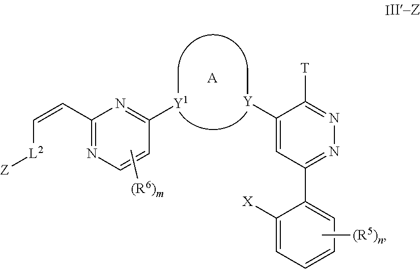

or a pharmaceutically acceptable salt, solvate, stereoisomer, or tautomer thereof,
wherein L², m, n, R⁵, R⁶, T, X, Y, Y¹, Z, and Ring A are each independently as defined herein.

In some embodiments, the compound of Formula III′-Z is represented by Formula IIIa′-Z:

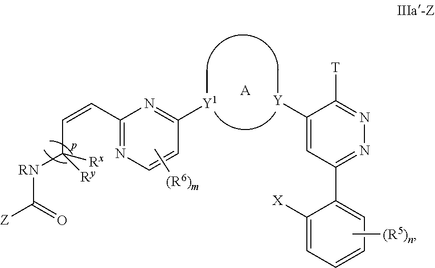

or a pharmaceutically acceptable salt, solvate, stereoisomer, or tautomer thereof,
wherein m, n, p, R, R⁵, R⁶, R^{x}, R^{y}, T, X, Y, Y¹, Z, and Ring A are each independently as defined herein.

In some embodiments, the compound of Formula III′-Z is represented by Formula IIIb′-Z:

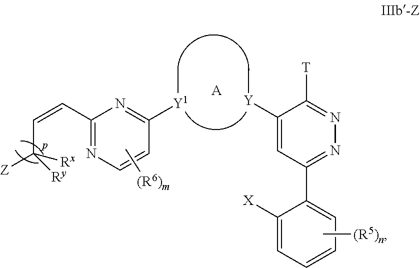

or a pharmaceutically acceptable salt, solvate, stereoisomer, or tautomer thereof,
wherein m, n, p, R⁵, R⁶, R^{x}, R^{y}, T, X, Y, Y¹, Z, and Ring A are each independently as defined herein.

In some embodiments of compounds of Formula I or Formula I′, or a pharmaceutically acceptable salt, solvate, stereoisomer, or tautomer thereof,

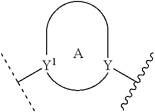

is a monocyclic nitrogen-containing 4- to 11-membered heterocycloalkyl. In some embodiments,

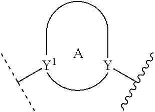

is selected from

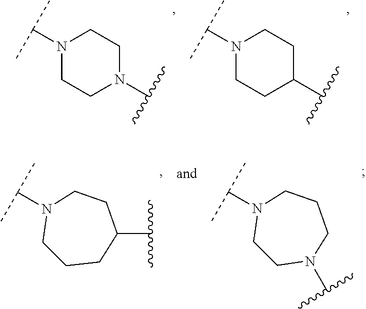

wherein the dashed line ( [symbol] ) denotes the point of attachment from Ring A to pyrimidine, and the wavy line ( [symbol] ) denotes the point of attachment from Ring A to L or pyridazine, and wherein each Ring A is independently unsubstituted or substituted with one to four R¹, wherein each R¹ is independently halo or C₁₋₄ alkyl.

In some embodiments of compounds of Formula I, or a pharmaceutically acceptable salt, solvate, stereoisomer, or tautomer thereof,

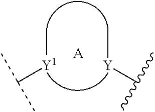

is a monocyclic nitrogen-containing 4- to 11-membered heterocycloalkyl. In some embodiments,

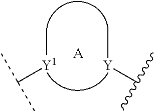

is selected from

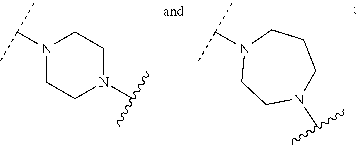

wherein the dashed line ( [symbol] ) denotes the point of attachment from Ring A to pyrimidine, and the wavy line ( [symbol] ) denotes the point of attachment from Ring A to pyridazine, and wherein each Ring A is independently unsubstituted or substituted with one to four R¹, wherein each R¹ is independently halo or C₁₋₄ alkyl.

In some embodiments of compounds of Formula I or Formula I′, or a pharmaceutically acceptable salt, solvate, stereoisomer, or tautomer thereof,

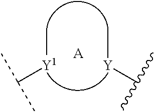

is a fused-bicyclic nitrogen-containing 4- to 11-membered heterocycloalkyl. In some embodiments,

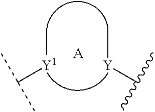

is selected from

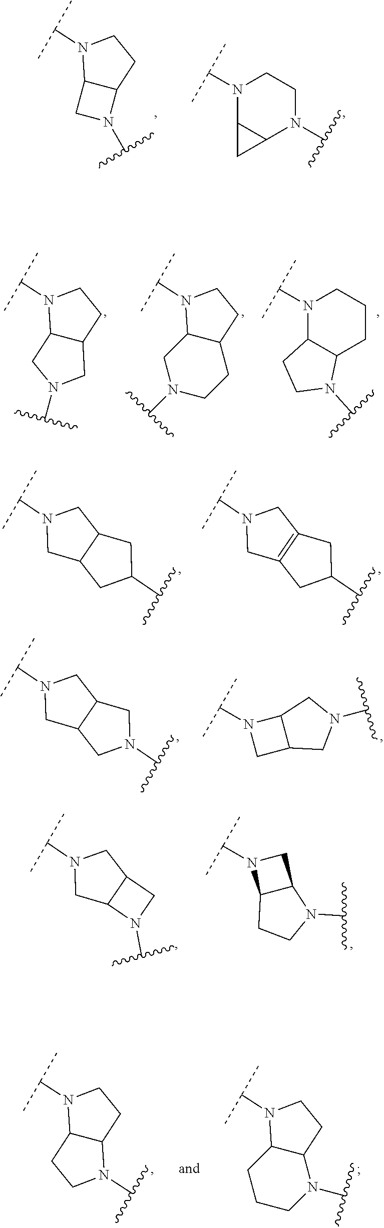

wherein the dashed line ( [symbol] ) denotes the point of attachment from Ring A to pyrimidine, and the wavy line ( [symbol] ) denotes the point of attachment from Ring A to L or pyridazine, and wherein each Ring A is independently unsubstituted or substituted with one to four R¹, wherein each R¹ is independently halo or C₁₋₄ alkyl.

In some embodiments of compounds of Formula I, or a pharmaceutically acceptable salt, solvate, stereoisomer, or tautomer thereof,

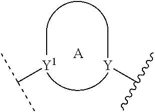

is a fused-bicyclic nitrogen-containing 4- to 11-membered heterocycloalkyl. In some embodiments,

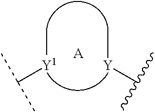

is selected from

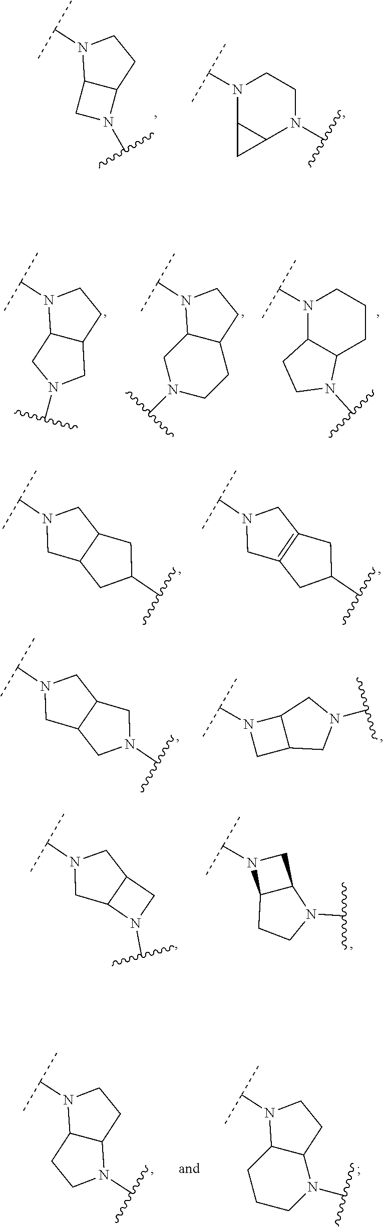

wherein the dashed line ( [symbol] ) denotes the point of attachment from Ring A to pyrimidine, and the wavy line ( [symbol] ) denotes the point of attachment from Ring A to pyridazine, and wherein each Ring A is independently unsubstituted or substituted with one to four R¹, wherein each R¹ is independently halo or C₁₋₄ alkyl.

In some embodiments of compounds of Formula I or Formula I′, or a pharmaceutically acceptable salt, solvate, stereoisomer, or tautomer thereof,

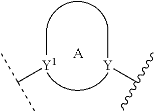

is a spirocyclic nitrogen-containing 4- to 11-membered heterocycloalkyl. In some embodiments,

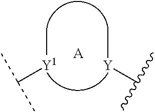

is selected from

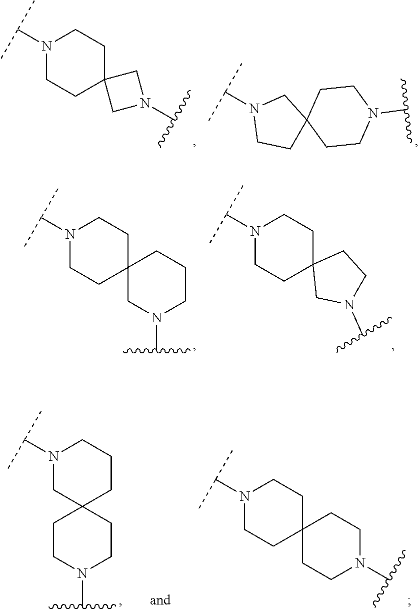

wherein the dashed line ( [symbol] ) denotes the point of attachment from Ring A to pyrimidine, and the wavy line ( [symbol] ) denotes the point of attachment from Ring A to L or pyridazine, and wherein each Ring A is independently unsubstituted or substituted with one to four R¹, wherein each R¹ is independently halo or C₁₋₄ alkyl.

In some embodiments of compounds of Formula I, or a pharmaceutically acceptable salt, solvate, stereoisomer, or tautomer thereof,

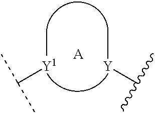

is a spirocyclic nitrogen-containing 4- to 11-membered heterocycloalkyl. In some embodiments,

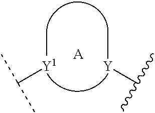

is selected from

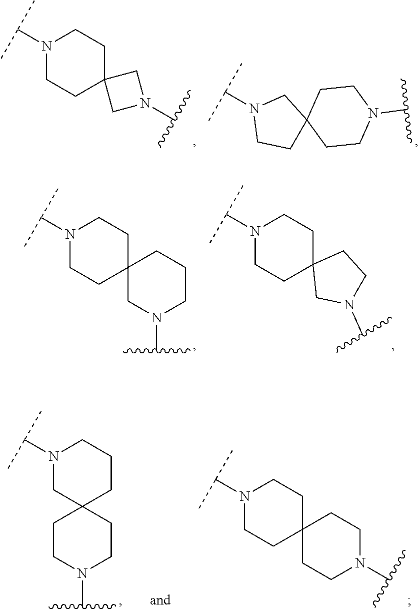

wherein the dashed line ( [symbol] ) denotes the point of attachment from Ring A to pyrimidine, and the wavy line ( [symbol] ) denotes the point of attachment from Ring A to pyridazine, and wherein each Ring A is independently unsubstituted or substituted with one to four R¹, wherein each R¹ is independently halo or C₁₋₄ alkyl.

In some embodiments of compounds of Formula I or Formula I′, or a pharmaceutically acceptable salt, solvate, stereoisomer, or tautomer thereof,

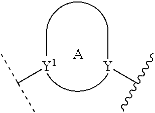

is a bridged nitrogen-containing 4- to 11-membered heterocycloalkyl. In some embodiments,

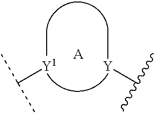

is selected from

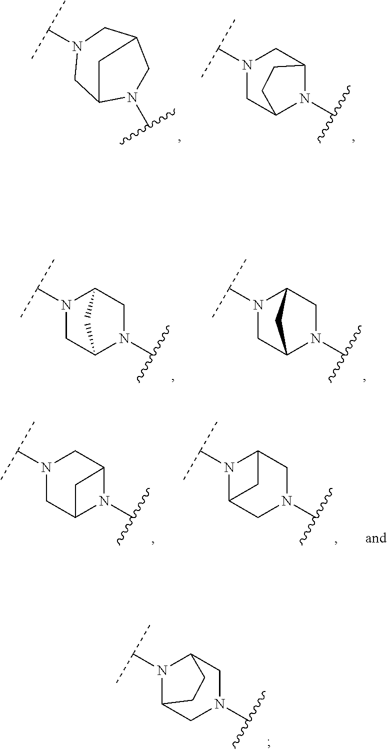

wherein the dashed line ( [symbol] ) denotes the point of attachment from Ring A to pyrimidine, and the wavy line ( [symbol] ) denotes the point of attachment from Ring A to L or pyridazine, and wherein each Ring A is independently unsubstituted or substituted with one to four R¹, wherein each R¹ is independently halo or C₁₋₄ alkyl.

In some embodiments of compounds of Formula I, or a pharmaceutically acceptable salt, solvate, stereoisomer, or tautomer thereof,

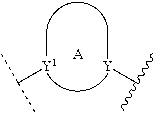

is a bridged nitrogen-containing 4- to 11-membered heterocycloalkyl. In some embodiments

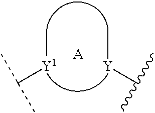

is selected from

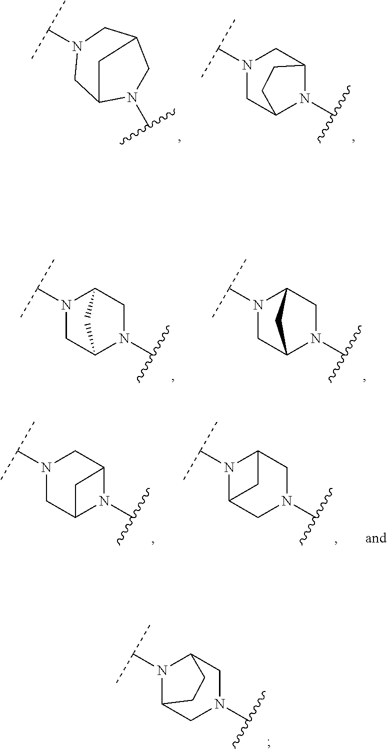

wherein the dashed line ( [symbol] ) denotes the point of attachment from Ring A to pyrimidine, and the wavy line ( [symbol] ) denotes the point of attachment from Ring A to pyridazine, and wherein each Ring A is independently unsubstituted or substituted with one to four R¹, wherein each R¹ is independently halo or C₁₋₄ alkyl.

In some embodiments of compounds of Formula I or Formula I′, or a pharmaceutically acceptable salt, solvate, stereoisomer, or tautomer thereof, Z is a nitrogen-containing 4- to 10-membered heterocycloalkyl. In some embodiments, Z is selected from

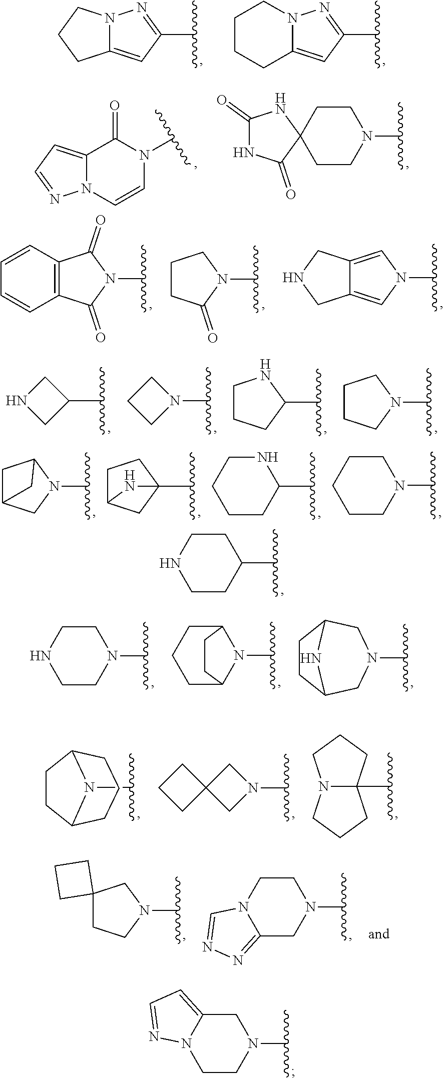

wherein each Z is independently unsubstituted or substituted with one to four R², wherein each R² is independently oxo, hydroxy, cyano, halo, —C(O)OR, —NRC(O)OR, C₁₋₄ alkyl, C₁₋₄ alkoxy, C₁₋₄ haloalkyl substituted with from 1 to 3 halo groups, C₅₋₆ heteroaryl having from 1 to 3 heteroatoms selected from O, N, and NR, or C₃₋₆ cycloalkyl.

In some embodiments of compounds of Formula I, or a pharmaceutically acceptable salt, solvate, stereoisomer, or tautomer thereof, Z is a nitrogen-containing 4- to 10-membered heterocycloalkyl. In some embodiments, Z is selected from

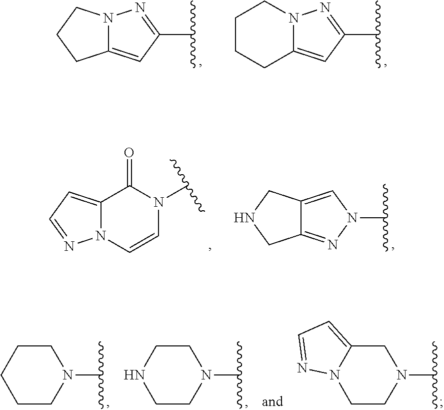

wherein each Z is independently unsubstituted or substituted with one to four R², wherein each R² is independently oxo, halo, —C(O)OR, —NRC(O)OR, C₁₋₄ alkyl, C₁₋₄ haloalkyl substituted with from 1 to 3 halo groups, C₅₋₆ heteroaryl having from 1 to 3 heteroatoms selected from O, N, and NR, or C₃₋₆ cycloalkyl.

In some embodiments of compounds of Formula I or Formula I′, or a pharmaceutically acceptable salt, solvate, stereoisomer, or tautomer thereof, Z is an oxygen- and nitrogen-containing 4- to 10-membered heterocycloalkyl. In some embodiments, Z is selected from

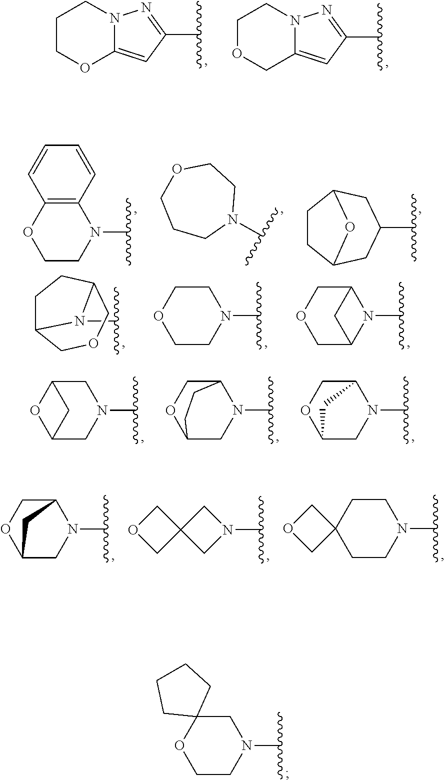

wherein each Z is independently unsubstituted or substituted with one to four R², wherein each R² is independently oxo, halo, —C(O)OR, —NRC(O)OR, C₁₋₄ alkyl, C₁₋₄ haloalkyl substituted with from 1 to 3 halo groups, C₅₋₆ heteroaryl having from 1 to 3 heteroatoms selected from O, N, and NR, or C₃₋₆ cycloalkyl.

In some embodiments of compounds of Formula I, or a pharmaceutically acceptable salt, solvate, stereoisomer, or tautomer thereof, Z is an oxygen- and nitrogen-containing 4- to 10-membered heterocycloalkyl. In some embodiments, Z is selected from

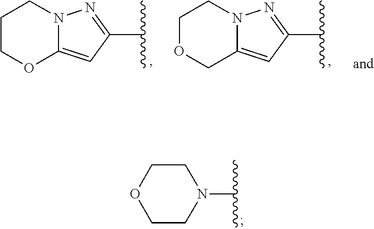

wherein each Z is independently unsubstituted or substituted with one to four R², wherein each R² is independently oxo, halo, —C(O)OR, —NRC(O)OR, C₁₋₄ alkyl, C₁₋₄ haloalkyl substituted with from 1 to 3 halo groups, C₅₋₆ heteroaryl having from 1 to 3 heteroatoms selected from O, N, and NR, or C₃₋₆ cycloalkyl.

In some embodiments of compounds of Formula I, or a pharmaceutically acceptable salt, solvate, stereoisomer, or tautomer thereof, Z is a sulfur- and nitrogen-containing 4- to 10-membered heterocycloalkyl. In some embodiments, Z is

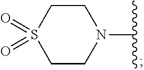

wherein Z is independently unsubstituted or substituted with one to four R², wherein each R² is independently oxo, halo, —C(O)OR, —NRC(O)OR, C₁₋₄ alkyl, C₁₋₄ haloalkyl substituted with from 1 to 3 halo groups, C₅₋₆ heteroaryl having from 1 to 3 heteroatoms selected from O, N, and NR, or C₃₋₆ cycloalkyl.

In some embodiments of compounds of Formula I or Formula I′, or a pharmaceutically acceptable salt, solvate, stereoisomer, or tautomer thereof, Z is an oxygen-containing 4- to 10-membered heterocycloalkyl. In some embodiments, Z is selected from

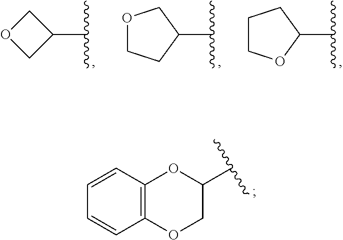

wherein Z is independently unsubstituted or substituted with one to four R², wherein each R² is independently oxo, halo, —C(O)OR, —NRC(O)OR, C₁₋₄ alkyl, C₁₋₄ haloalkyl substituted with from 1 to 3 halo groups, C₅₋₆ heteroaryl having from 1 to 3 heteroatoms selected from O, N, and NR, or C₃₋₆ cycloalkyl.

In some embodiments of compounds of Formula I, or a pharmaceutically acceptable salt, solvate, stereoisomer, or tautomer thereof, Z is an oxygen-containing 4- to 10-membered heterocycloalkyl. In some embodiments, Z is

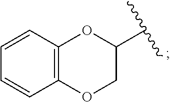

wherein Z is independently unsubstituted or substituted with one to four R², wherein each R² is independently oxo, halo, —C(O)OR, —NRC(O)OR, C₁₋₄ alkyl, C₁₋₄ haloalkyl substituted with from 1 to 3 halo groups, C₅₋₆ heteroaryl having from 1 to 3 heteroatoms selected from O, N, and NR, or C₃₋₆ cycloalkyl.

In some embodiments of compounds of Formula I or Formula I′, or a pharmaceutically acceptable salt, solvate, stereoisomer, or tautomer thereof, Z is a nitrogen-containing 5- to 10-membered heteroaryl.

In some embodiments, Z is selected from

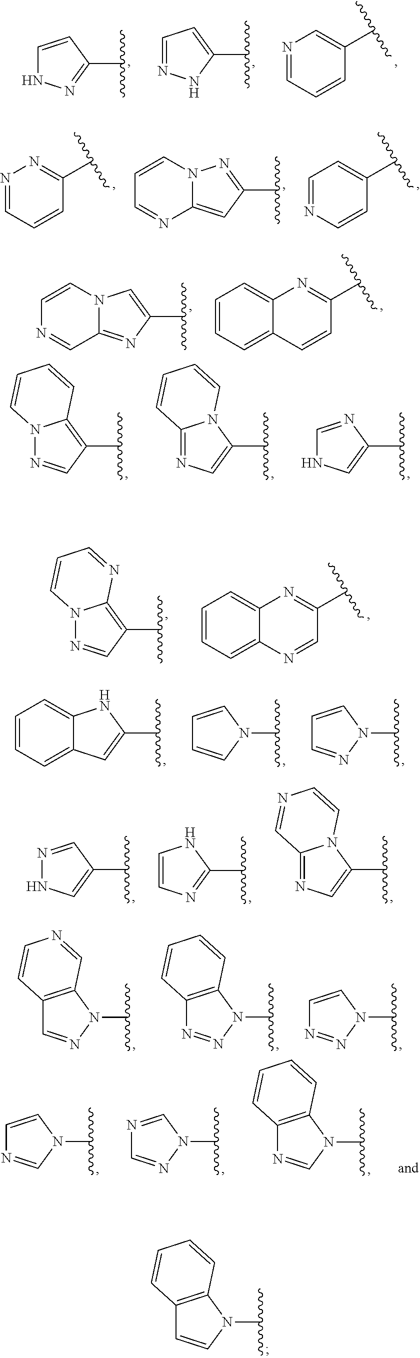

wherein each Z is independently unsubstituted or substituted with one to four R², wherein each R² is independently oxo, halo, —C(O)OR, —NRC(O)OR, C₁₋₄ alkyl, C₁₋₄ haloalkyl substituted with from 1 to 3 halo groups, C₅₋₆ heteroaryl having from 1 to 3 heteroatoms selected from O, N, and NR, or C₃₋₆ cycloalkyl.

In some embodiments of compounds of Formula I, or a pharmaceutically acceptable salt, solvate, stereoisomer, or tautomer thereof, Z is a nitrogen-containing 5- to 10-membered heteroaryl. In some embodiments, Z is selected from

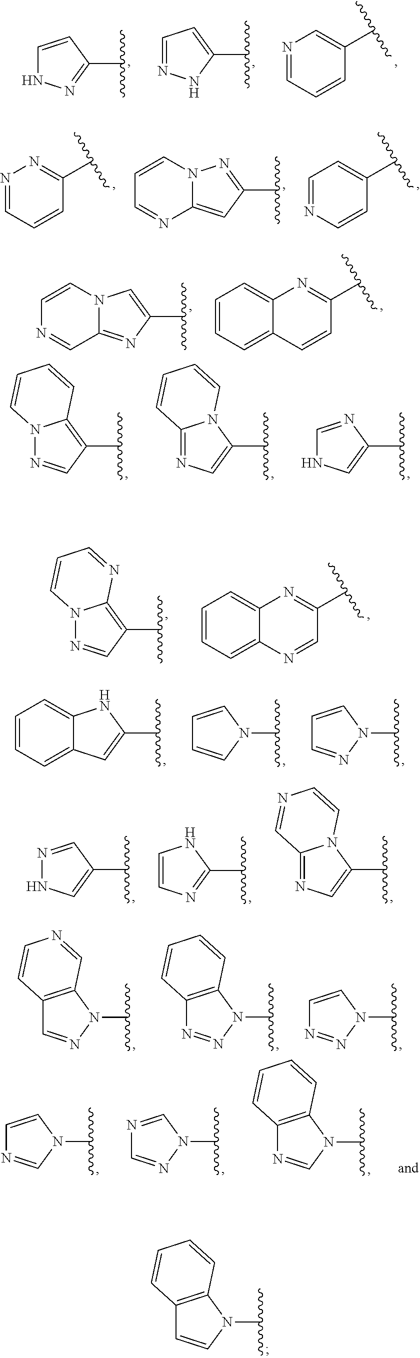

wherein each Z is independently unsubstituted or substituted with one to four R², wherein each R² is independently oxo, halo, —C(O)OR, —NRC(O)OR, C₁₋₄ alkyl, C₁₋₄ haloalkyl substituted with from 1 to 3 halo groups, C₅₋₆ heteroaryl having from 1 to 3 heteroatoms selected from O, N, and NR, or C₃₋₆ cycloalkyl.

In some embodiments of compounds of Formula I, or a pharmaceutically acceptable salt, solvate, stereoisomer, or tautomer thereof, Z is an oxygen- and nitrogen-containing 5- to 10-membered heteroaryl. In some embodiments, Z is selected from

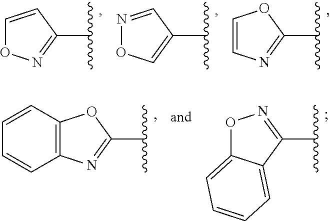

wherein each Z is independently unsubstituted or substituted with one to four R², wherein each R² is independently oxo, halo, —C(O)OR, —NRC(O)OR, C₁₋₄ alkyl, C₁₋₄ haloalkyl substituted with from 1 to 3 halo groups, C₅₋₆ heteroaryl having from 1 to 3 heteroatoms selected from O, N, and NR, or C₃₋₆ cycloalkyl.

In some embodiments of compounds of Formula I, or a pharmaceutically acceptable salt, solvate, stereoisomer, or tautomer thereof, Z is a sulfur- and nitrogen-containing 5- to 10-membered heteroaryl. In some embodiments, Z is selected from

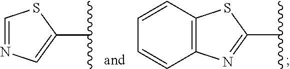

wherein each Z is independently unsubstituted or substituted with one to four R², wherein each R² is independently oxo, halo, —C(O)OR, —NRC(O)OR, C₁₋₄ alkyl, C₁₋₄ haloalkyl substituted with from 1 to 3 halo groups, C₅₋₆ heteroaryl having from 1 to 3 heteroatoms selected from O, N, and NR, or C₃₋₆ cycloalkyl.

In some embodiments of compounds of Formula I, or a pharmaceutically acceptable salt, solvate, stereoisomer, or tautomer thereof, Z is an oxygen-containing 5- to 10-membered heteroaryl. In some embodiments, Z is selected from

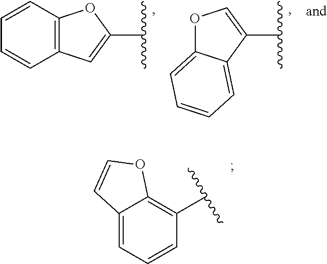

wherein each Z is independently unsubstituted or substituted with one to four R², wherein each R² is independently oxo, halo, —C(O)OR, —NRC(O)OR, C₁₋₄ alkyl, C₁₋₄ haloalkyl substituted with from 1 to 3 halo groups, C₅₋₆ heteroaryl having from 1 to 3 heteroatoms selected from O, N, and NR, or C₃₋₆ cycloalkyl.

In some embodiments of compounds of Formula I, or a pharmaceutically acceptable salt, solvate, stereoisomer, or tautomer thereof, Z is C₁₋₁₀ alkyl. In some embodiments of compounds of Formula I, or a pharmaceutically acceptable salt, solvate, stereoisomer, or tautomer thereof, Z is —N(R)₂. In some embodiments of compounds of Formula I, or a pharmaceutically acceptable salt, solvate, stereoisomer, or tautomer thereof, Z is C₁₋₁₀ alkoxy. In some embodiments of compounds of Formula I, or a pharmaceutically acceptable salt, solvate, stereoisomer, or tautomer thereof, Z is —OH.

In some embodiments, provided is a compound of Formula I, wherein X is hydroxy.

In some embodiments, provided is a compound of Formula I, wherein T is —NH₂.

In some embodiments, provided is a compound of Formula I, wherein Y is N. In some embodiments, provided is a compound of Formula I, wherein Y¹ is N. In some embodiments, provided is a compound of Formula I, wherein both Y and Y¹ are N.

In some embodiments, provided is a compound of Formula T, wherein L² is

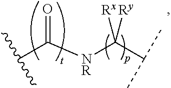

wherein t is 0 or 1, p is 1, 2, or 3, and wherein the dashed line ( [symbol] ) denotes the point of attachment from L² to L¹, and the wavy line ( [symbol] ) denotes the point of attachment from L² to Z. In some embodiments, provided is a compound of Formula I, wherein L² is

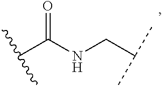

wherein the dashed line ( [symbol] ) denotes the point of attachment from L² to L¹, and the wavy line ( [symbol] ) denotes the point of attachment from L² to Z.

Representative and non-limiting examples of compounds within the scope of Formula I above are set forth in Table 1, where each of which include their pharmaceutically acceptable salts, solvates, hydrates, or tautomers thereof.

TABLE 1

| Compound | | |
|---|---|---|
| # | Structure | IUPAC Name |
| P-1 | 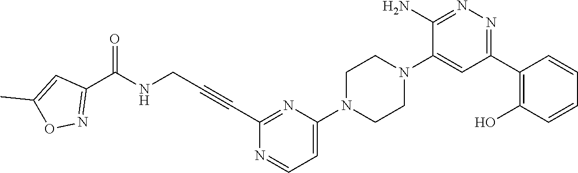 [SMILES: Cc1cc(C(=O)NCC#Cc2nccc(N3CCN(c4cc(-c5ccccc5O)nnc4N)CC3)n2)no1] | N-[3-(4-{4-[3-amino-6-(2- hydroxyphenyl)pyridazin-4-yl]piperazin- 1-yl}pyrimidin-2-yl)prop-2-yn-1-yl]-5- methyl-1,2-oxazole-3-carboxamide |
| P-2 | 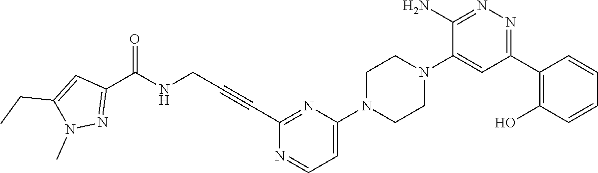 [SMILES: CCc1cc(C(=O)NCC#Cc2nccc(N3CCN(c4cc(-c5ccccc5O)nnc4N)CC3)n2)nn1C] | N-[3-(4-{4-[3-amino-6-(2- hydroxyphenyl)pyridazin-4-yl\|piperazin- 1-yl}pyrimidin-2-yl)prop-2-yn-1-yl]-5- ethyl-1-methyl-1H-pyrazole-3- carboxamide |
| P-3 | 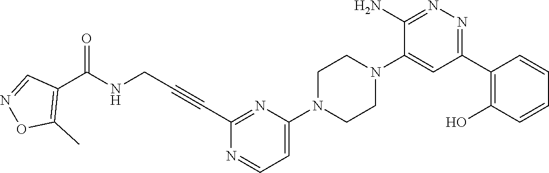 [SMILES: Cc1oncc1C(=O)NCC#Cc1nccc(N2CCN(c3cc(-c4ccccc4O)nnc3N)CC2)n1] | N-[3-(4-{4-[3-amino-6-(2- hydroxyphenyl)pyridazin-4-yl]piperazin- 1-yl}pyrimidin-2-yl)prop-2-yn-1-yl]-5- methyl-1,2-oxazole-4-carboxamide |
| P-4 | 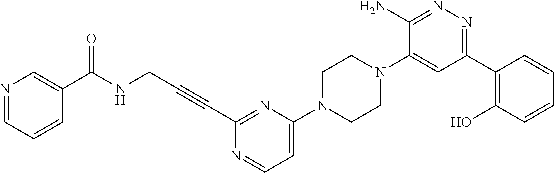 [SMILES: Nc1nnc(-c2ccccc2O)cc1N1CCN(c2ccnc(C#CCNC(=O)c3cccnc3)n2)CC1] | N-[3-(4-{4-[3-amino-6-(2- hydroxyphenyl)pyridazin-4-yl]piperazin- 1-yl}pyrimidin-2-yl)prop-2-yn-1- yl]pyridine-3-carboxamide |
| P-5 | 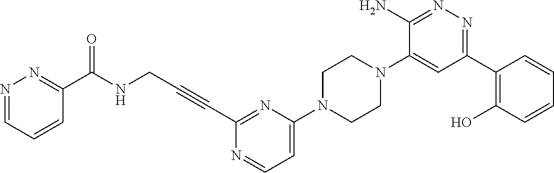 [SMILES: Nc1nnc(-c2ccccc2O)cc1N1CCN(c2ccnc(C#CCNC(=O)c3cccnn3)n2)CC1] | N-[3-(4-{4-[3-amino-6-(2- hydroxyphenyl)pyridazin-4-yl]piperazin- 1-yl}pyrimidin-2-yl)prop-2-yn-1- yl]pyridazine-3-carboxamide |
| P-6 | 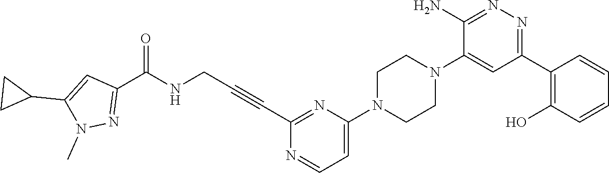 [SMILES: Cn1nc(C(=O)NCC#Cc2nccc(N3CCN(c4cc(-c5ccccc5O)nnc4N)CC3)n2)cc1C1CC1] | N-[3-(4-{4-[3-amino-6-(2- hydroxyphenyl)pyridazin-4-yl]piperazin- 1-yl}pyrimidin-2-yl)prop-2-yn-1-yl]-5- cyclopropyl-1-methyl-1H-pyrazole-3- carboxamide |
| P-7 | 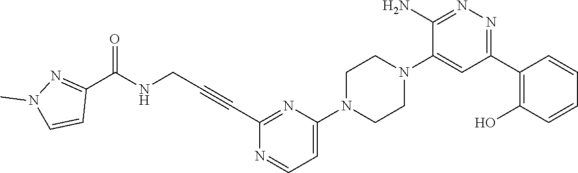 [SMILES: Cn1ccc(C(=O)NCC#Cc2nccc(N3CCN(c4cc(-c5ccccc5O)nnc4N)CC3)n2)n1] | N-[3-(4-{4-[3-amino-6-(2- hydroxyphenyl)pyridazin-4-yl]piperazin- 1-yl}pyrimidin-2-yl)prop-2-yn-1-yl]-1- methyl-1H-pyrazole-3-carboxamide |
| P-8 | 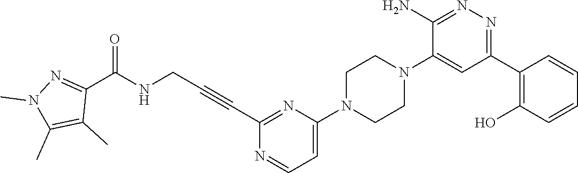 [SMILES: Cc1c(C(=O)NCC#Cc2nccc(N3CCN(c4cc(-c5ccccc5O)nnc4N)CC3)n2)nn(C)c1C] | N-[3-(4-{4-[3-amino-6-(2- hydroxyphenyl)pyridazin-4-yl]piperazin- 1-yl}pyrimidin-2-yl)prop-2-yn-1-yl]- 1,4,5-trimethyl-1H-pyrazole-3- carboxamide |
| P-9 | 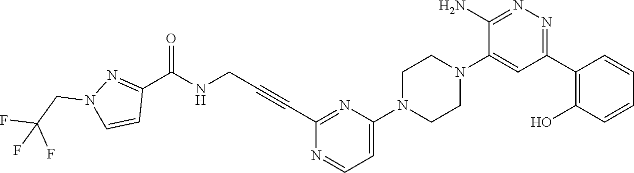 [SMILES: Nc1nnc(-c2ccccc2O)cc1N1CCN(c2ccnc(C#CCNC(=O)c3ccn(CC(F)(F)F)n3)n2)CC1] | N-[3-(4-{ 4-[3-amino-6-(2- hydroxyphenyl)pyridazin-4-yl]piperazin- 1-yl}pyrimidin-2-yl)prop-2-yn-1-yl]-1- (2,2,2-trifluoroethyl)-1H-pyrazole-3- carboxamide |
| P-10 | 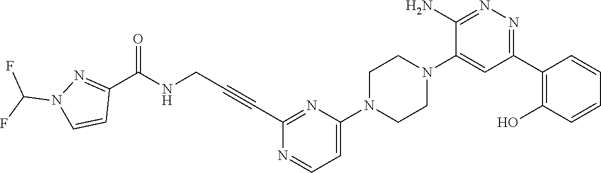 [SMILES: Nc1nnc(-c2ccccc2O)cc1N1CCN(c2ccnc(C#CCNC(=O)c3ccn(C(F)F)n3)n2)CC1] | N-[3-(4-{4-[3-amino-6-(2- hydroxyphenyl)pyridazin-4-yl]piperazin- 1-yl}pyrimidin-2-yl)prop-2-yn-1-yl]-1- (difluoromethyl)-1H-pyrazole-3- carboxamide |
| P-11 | 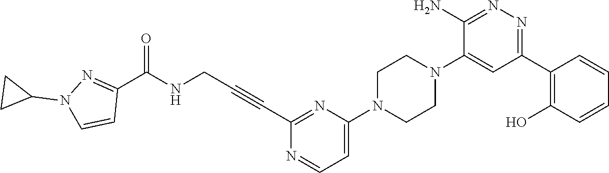 [SMILES: Nc1nnc(-c2ccccc2O)cc1N1CCN(c2ccnc(C#CCNC(=O)c3ccn(C4CC4)n3)n2)CC1] | N-[3-(4-{ 4-[3-amino-6-(2- hydroxyphenyl)pyridazin-4-yl]piperazin- 1-yl}pyrimidin-2-yl)prop-2-yn-1-yl]-1- cyclopropyl-1H-pyrazole-3-carboxamide |
| P-12 | 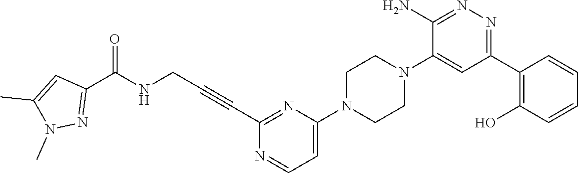 [SMILES: Cc1cc(C(=O)NCC#Cc2nccc(N3CCN(c4cc(-c5ccccc5O)nnc4N)CC3)n2)nn1C] | N-[3-(4-{4-[3-amino-6-(2- hydroxyphenyl)pyridazin-4-yl]piperazin- 1-yl}pyrimidin-2-yl)prop-2-yn-1-yl]-1,5- dimethyl-1H-pyrazole-3-carboxamide |
| P-14 | 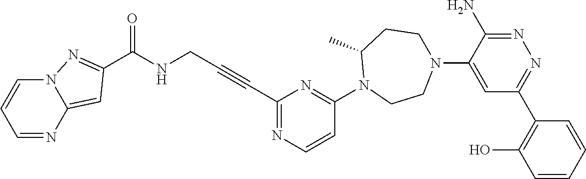 [SMILES: C[C@@H]1CCN(c2cc(-c3ccccc3O)nnc2N)CCN1c1ccnc(C#CCNC(=O)c2cc3ncccn3n2)n1] | (R)-N-(3-(4-(4-(3-amino-6-(2- hydroxyphenyl)pyridazin-4-yl)-7-methyl- 1,4-diazepan-1-yl)pyrimidin-2-yl)prop-2- yn-1-yl)pyrazolo[1,5-a]pyrimidine-2- carboxamide |
| P-15 | 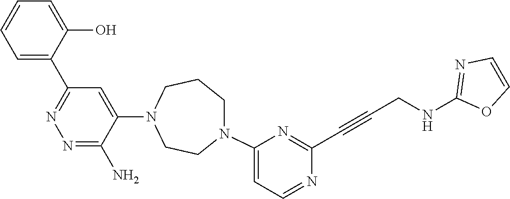 [SMILES: Nc1nnc(-c2ccccc2O)cc1N1CCCN(c2ccnc(C#CCNc3ncco3)n2)CC1] | 2-(6-amino-5-(4-(2-(3-(oxazol-2- ylamino)prop-1-yn-1-yl)pyrimidin-4-yl)- 1,4-diazepan-1-yl)pyridazin-3-yl)phenol |
| P-16 | 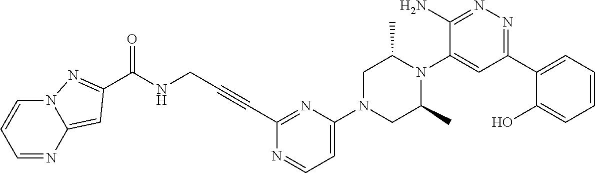 [SMILES: C[C@H]1CN(c2ccnc(C#CCNC(=O)c3cc4ncccn4n3)n2)C[C@H](C)N1c1cc(-c2ccccc2O)nnc1N] | N-(3-(4-((3S,5S)-4-(3-amino-6-(2- hydroxyphenyl)pyridazin-4-yl)-3,5- dimethylpiperazin-1-yl)pyrimidin-2- yl)prop-2-yn-1-yl)pyrazolo[1,5- a]pyrimidine-2-carboxamide |
| P-17 | 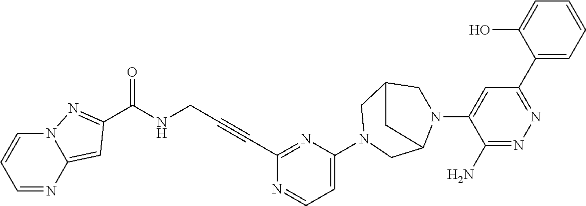 [SMILES: Nc1nnc(-c2ccccc2O)cc1N1CC2CC1CN(c1ccnc(C#CCNC(=O)c3cc4ncccn4n3)n1)C2] | N-(3-(4-(6-(3-amino-6-(2- hydroxyphenyl)pyridazin-4-yl)-3,6- diazabicyclo[3.2.1]octan-3-yl)pyrimidin- 2-yl)prop-2-yn-1-yl)pyrazolo[1,5- a]pyrimidine-2-carboxamide |
| P-18 | 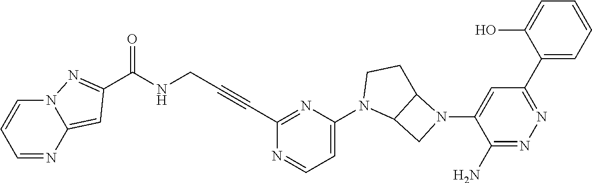 [SMILES: Nc1nnc(-c2ccccc2O)cc1N1CC2C1CCN2c1ccnc(C#CCNC(=O)c2cc3ncccn3n2)n1] | N-(3-(4-(6-(3-amino-6-(2- hydroxyphenyl)pyridazin-4-yl)-2,6- diazabicyclo[3.2.0]heptan-2-yl)pyrimidin- 2-yl)prop-2-yn-1-yl)pyrazolo[1,5- a]pyrimidine-2-carboxamide |
| P-19 | 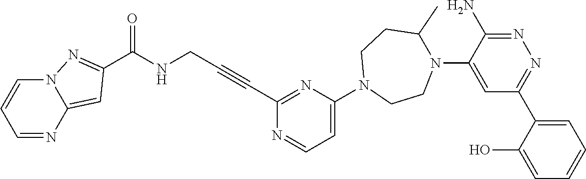 [SMILES: CC1CCN(c2ccnc(C#CCNC(=O)c3cc4ncccn4n3)n2)CCN1c1cc(-c2ccccc2O)nnc1N] | N-(3-(4-(4-(3-amino-6-(2- hydroxyphenyl)pyridazin-4-yl)-5-methyl- 1,4-diazepan-1-yl)pyrimidin-2-yl)prop-2- yn-1-yl)pyrazolo[1,5-a]pyrimidine-2- carboxamide |
| P-20 | 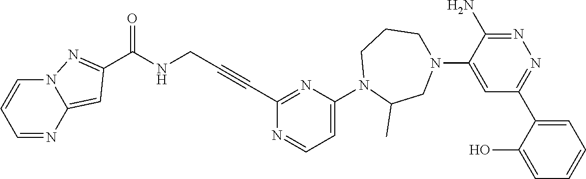 [SMILES: CC1CN(c2cc(-c3ccccc3O)nnc2N)CCCN1c1ccnc(C#CCNC(=O)c2cc3ncccn3n2)n1] | N-(3-(4-(4-(3-amino-6-(2- hydroxyphenyl)pyridazin-4-yl)-2-methyl- 1,4-diazepan-1-yl)pyrimidin-2-yl)prop-2- yn-1-yl)pyrazolo[1,5-a]pyrimidine-2- carboxamide |
| P-21 | 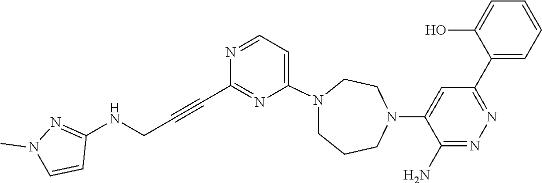 [SMILES: Cn1ccc(NCC#Cc2nccc(N3CCCN(c4cc(-c5ccccc5O)nnc4N)CC3)n2)n1] | 2-(6-amino-5-(4-(2-(3-((1-methyl-1H- pyrazol-3-yl)amino)prop-1-yn-1- yl)pyrimidin-4-yl)-1,4-diazepan-1- yl)pyridazin-3-yl)phenol |
| P-22 | 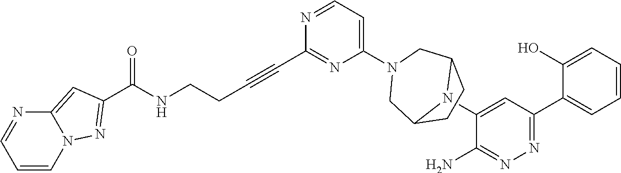 [SMILES: Nc1nnc(-c2ccccc2O)cc1N1C2CCC1CN(c1ccnc(C#CCCNC(=O)c3cc4ncccn4n3)n1)C2] | N-(4-(4-(8-(3-amino-6-(2- hydroxyphenyl)pyridazin-4-yl)-3,8- diazabicyclo[3.2.1]octan-3-yl)pyrimidin- 2-yl)but-3-yn-1-yl)pyrazolo[1,5- a]pyrimidine-2-carboxamide |
| P-23 | 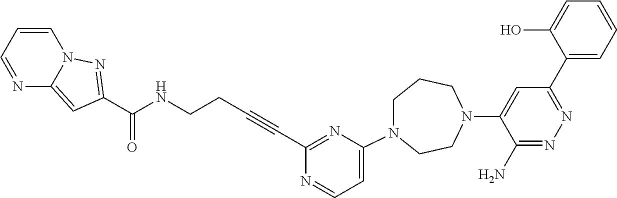 [SMILES: Nc1nnc(-c2ccccc2O)cc1N1CCCN(c2ccnc(C#CCCNC(=O)c3cc4ncccn4n3)n2)CC1] | N-(4-(4-(4-(3-amino-6-(2- hydroxyphenyl)pyridazin-4-yl)-1,4- diazepan-1-yl)pyrimidin-2-yl)but-3-yn-1- yl)pyrazolo[1,5-a]pyrimidine-2- carboxamide |
| P-24 | 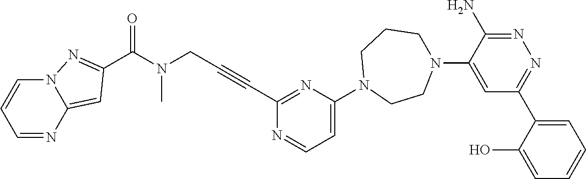 [SMILES: CN(CC#Cc1nccc(N2CCCN(c3cc(-c4ccccc4O)nnc3N)CC2)n1)C(=O)c1cc2ncccn2n1] | N-(3-(4-(4-(3-amino-6-(2- hydroxyphenyl)pyridazin-4-yl)-1,4- diazepan-1-yl)pyrimidin-2-yl)prop-2-yn- 1-yl)-N-methylpyrazolo[1,5- a]pyrimidine-2-carboxamide |
| P-25 | 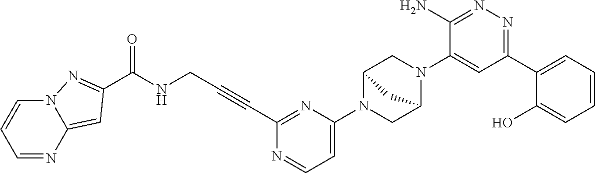 [SMILES: Nc1nnc(-c2ccccc2O)cc1N1C[C@H]2C[C@@H]1CN2c1ccnc(C#CCNC(=O)c2cc3ncccn3n2)n1] | N-(3-(4-((1R,4R)-5-(3-amino-6-(2- hydroxyphenyl)pyridazin-4-yl)-2,5- diazabicyclo[2.2.1]heptan-2-yl)pyrimidin- 2-yl)prop-2-yn-1-yl)pyrazolo[1,5- a]pyrimidine-2-carboxamide |
| P-26 | 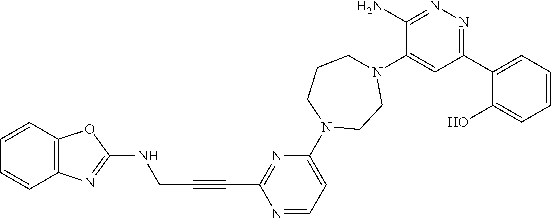 [SMILES: Nc1nnc(-c2ccccc2O)cc1N1CCCN(c2ccnc(C#CCNc3nc4ccccc4o3)n2)CC1] | 2-(6-amino-5-(4-(2-(3-(benzo[d]oxazol-2- ylamino)prop-1-yn-1-yl)pyrimidin-4-yl)- 1,4-diazepan-1-yl)pyridazin-3-yl)phenol |
| P-27 | 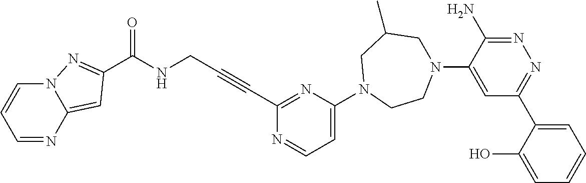 [SMILES: CC1CN(c2ccnc(C#CCNC(=O)c3cc4ncccn4n3)n2)CCN(c2cc(-c3ccccc3O)nnc2N)C1] | N-(3-(4-(4-(3-amino-6-(2- hydroxyphenyl)pyridazin-4-yl)-6-methyl- 1,4-diazepan-1-yl)pyrimidin-2-yl)prop-2- yn-1-yl)pyrazolo[1,5-a]pyrimidine-2- carboxamide |
| P-28 | 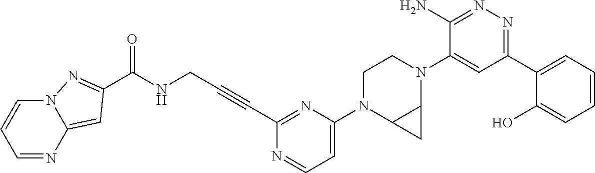 [SMILES: Nc1nnc(-c2ccccc2O)cc1N1CCN(c2ccnc(C#CCNC(=O)c3cc4ncccn4n3)n2)C2CC21] | N-(3-(4-(5-(3-amino-6-(2- hydroxyphenyl)pyridazin-4-yl)-2,5- diazabicyclo[4.1.0]heptan-2-yl)pyrimidin- 2-yl)prop-2-yn-1-yl)pyrazolo[1,5- a]pyrimidine-2-carboxamide |
| P-29 | 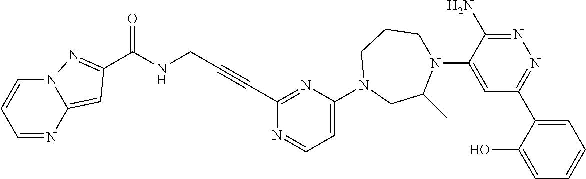 [SMILES: CC1CN(c2ccnc(C#CCNC(=O)c3cc4ncccn4n3)n2)CCCN1c1cc(-c2ccccc2O)nnc1N] | N-(3-(4-(4-(3-amino-6-(2- hydroxyphenyl)pyridazin-4-yl)-3-methyl- 1,4-diazcpan-1-yl)pyrimidin-2-yl)prop-2- yn-1-yl)pyrazolo[1,5-a]pyrimidine-2- carboxamide |
| P-30 | 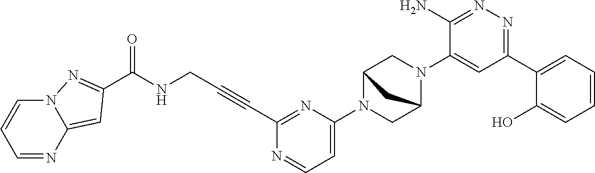 [SMILES: Nc1nnc(-c2ccccc2O)cc1N1C[C@@H]2C[C@H]1CN2c1ccnc(C#CCNC(=O)c2cc3ncccn3n2)n1] | N-(3-(4-((1S,4S)-5-(3-amino-6-(2- hydroxyphenyl)pyridazin-4-yl)-2,5- diazabicyclo[2.2.1]heptan-2-yl)pyrimidin- 2-yl)prop-2-yn-1-yl)pyrazolo[1,5- a]pyrimidine-2-carboxamide |
| P-31 | 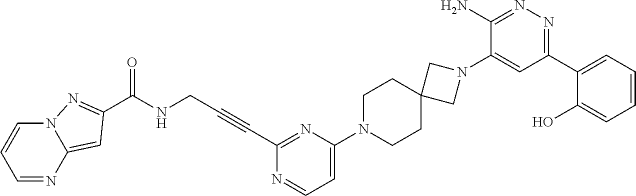 [SMILES: Nc1nnc(-c2ccccc2O)cc1N1CC2(CCN(c3ccnc(C#CCNC(=O)c4cc5ncccn5n4)n3)CC2)C1] | N-(3-(4-(2-(3-amino-6-(2- hydroxyphenyl)pyridazin-4-yl)-2,7- diazaspiro[3.5]nonan-7-yl)pyrimidin-2- yl)prop-2-yn-1-yl)pyrazolo[1,5- a]pyrimidine-2-carboxamide |
| P-32 | 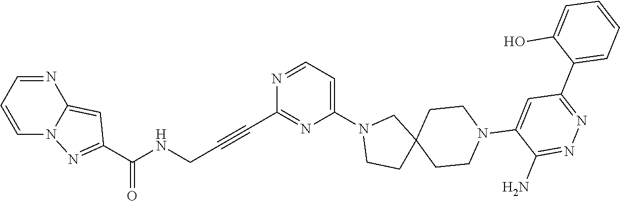 [SMILES: Nc1nnc(-c2ccccc2O)cc1N1CCC2(CCN(c3ccnc(C#CCNC(=O)c4cc5ncccn5n4)n3)C2)CC1] | N-(3-(4-(8-(3-amino-6-(2- hydroxyphenyl)pyridazin-4-yl)-2,8- diazaspiro[4.5]decan-2-yl)pyrimidin-2- yl)prop-2-yn-1-yl)pyrazolo[1,5- a]pyrimidine-2-carboxamide |
| P-33 | 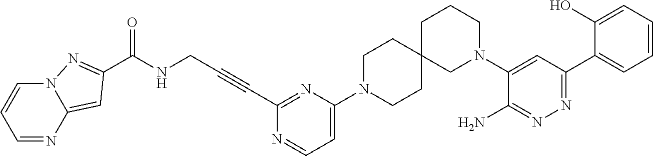 [SMILES: Nc1nnc(-c2ccccc2O)cc1N1CCCC2(CCN(c3ccnc(C#CCNC(=O)c4cc5ncccn5n4)n3)CC2)C1] | N-(3-(4-(2-(3-amino-6-(2- hydroxyphenyl)pyridazin-4-yl)-2,9- diazaspiro[5.5]undecan-9-yl)pyrimidin-2- yl)prop-2-yn-1-yl)pyrazolo[1,5- a]pyrimidine-2-carboxamide |
| P-34 | 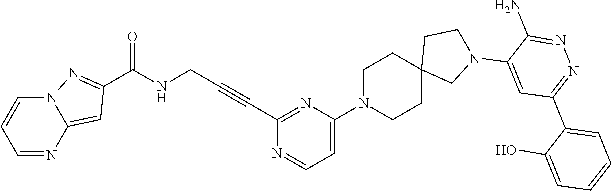 [SMILES: Nc1nnc(-c2ccccc2O)cc1N1CCC2(CCN(c3ccnc(C#CCNC(=O)c4cc5ncccn5n4)n3)CC2)C1] | N-(3-(4-(2-(3-amino-6-(2- hydroxyphenyl)pyridazin-4-yl)-2,8- diazaspiro[4.5]decan-8-yl)pyrimidin-2- yl)prop-2-yn-1-yl)pyrazolo[1,5- a]pyrimidine-2-carboxamide |
| P-35 | 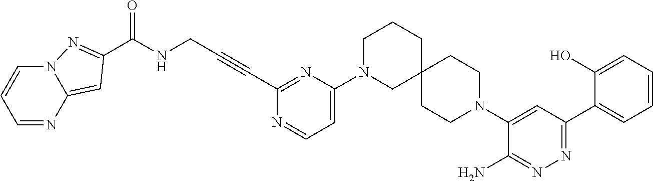 [SMILES: Nc1nnc(-c2ccccc2O)cc1N1CCC2(CCCN(c3ccnc(C#CCNC(=O)c4cc5ncccn5n4)n3)C2)CC1] | N-(3-(4-(9-(3-amino-6-(2- hydroxyphenyl)pyridazin-4-yl)-2,9- diazaspiro[5.5]undecan-2-yl)pyrimidin-2- yl)prop-2-yn-1-yl)pyrazolo[1,5- a]pyrimidine-2-carboxamide |
| P-36 | 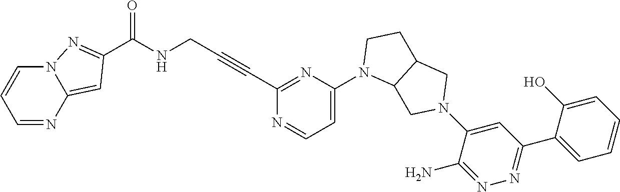 [SMILES: Nc1nnc(-c2ccccc2O)cc1N1CC2CCN(c3ccnc(C#CCNC(=O)c4cc5ncccn5n4)n3)C2C1] | N-(3-(4-(5-(3-amino-6-(2- hydroxyphenyl)pyridazin-4- yl)hexahydropyrrolo[3,4-b]pyrrol-1(2H)- yl)pyrimidin-2-yl)prop-2-yn-1- yl)pyrazolo[1,5-a]pyrimidine-2- carboxamide |
| P-37 | 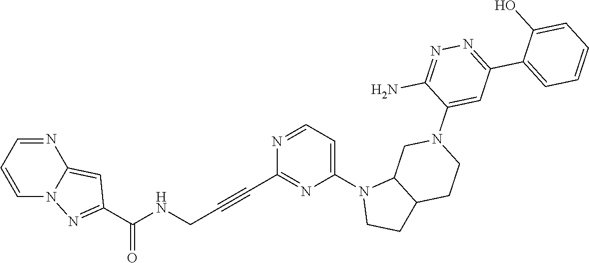 [SMILES: Nc1nnc(-c2ccccc2O)cc1N1CCC2CCN(c3ccnc(C#CCNC(=O)c4cc5ncccn5n4)n3)C2C1] | N-(3-(4-(6-(3-amino-6-(2- hydroxyphenyl)pyridazin-4-yl)octahydro- 1H-pyrrolo[2,3-c]pyridin-1-yl)pyrimidin- 2-yl)prop-2-yn-1-yl)pyrazolo[1,5- a]pyrimidine-2-carboxamide |
| P-38 | 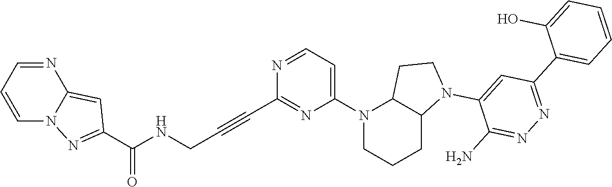 [SMILES: Nc1nnc(-c2ccccc2O)cc1N1CCC2C1CCCN2c1ccnc(C#CCNC(=O)c2cc3ncccn3n2)n1] | N-(3-(4-(1-(3-amino-6-(2- hydroxyphenyl)pyridazin-4-yl)octahydro- 4H-pyrrolo[3,2-b]pyridin-4-yl)pyrimidin- 2-yl)prop-2-yn-1-yl)pyrazolo[1,5- a]pyrimidine-2-carboxamide |
| P-39 | 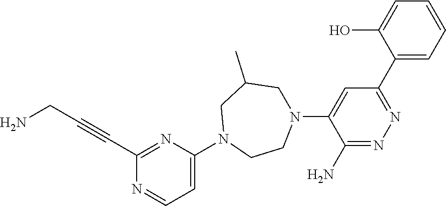 [SMILES: CC1CN(c2ccnc(C#CCN)n2)CCN(c2cc(-c3ccccc3O)nnc2N)C1] | 2-(6-amino-5-(4-(2-(3-aminoprop-1-yn-1- yl)pyrimidin-4-yl)-6-methyl-1,4- diazepan-1-yl)pyridazin-3-yl)phenol |
| P-40 | 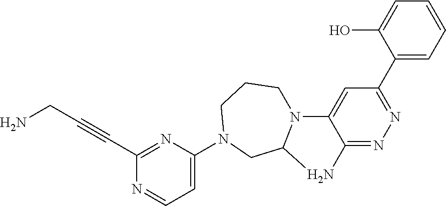 [SMILES: NCC#Cc1nccc(N2CCCN3c4cc(-c5ccccc5O)nnc4NC3C2)n1] | 2-(6-amino-5-(4-(2-(3-aminoprop-1-yn-1- yl)pyrimidin-4-yl)-2-methyl-1,4- diazepan-1-yl)pyridazin-3-yl)phenol |
| P-41 | 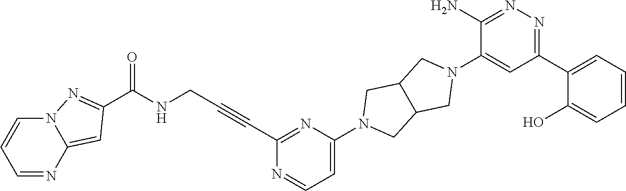 [SMILES: Nc1nnc(-c2ccccc2O)cc1N1CC2CN(c3ccnc(C#CCNC(=O)c4cc5ncccn5n4)n3)CC2C1] | N-(3-(4-(5-(3-amino-6-(2- hydroxyphenyl)pyridazin-4- yl)hexahydropyrrolo[3,4-c]pyrrol-2(1H)- yl)pyrimidin-2-yl)prop-2-yn-1- yl)pyrazolo[1,5-a]pyrimidine-2- carboxamide |
| P-42 | 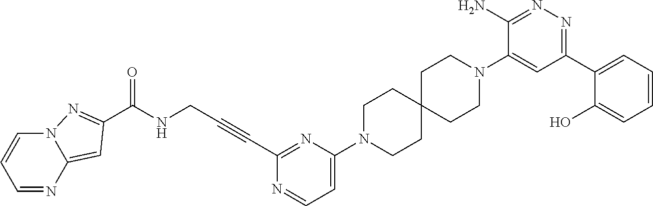 [SMILES: Nc1nnc(-c2ccccc2O)cc1N1CCC2(CCN(c3ccnc(C#CCNC(=O)c4cc5ncccn5n4)n3)CC2)CC1] | N-(3-(4-(9-(3-amino-6-(2- hydroxyphenyl)pyridazin-4-yl)-3,9- diazaspiro[5.5]undecan-3-yl)pyrimidin-2- yl)prop-2-yn-1-yl)pyrazolo[1,5- a]pyrimidine-2-carboxamide |
| P-43 | 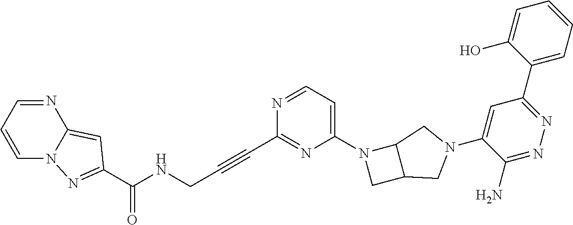 [SMILES: Nc1nnc(-c2ccccc2O)cc1N1CC2CN(c3ccnc(C#CCNC(=O)c4cc5ncccn5n4)n3)C2C1] | N-(3-(4-(3-(3-amino-6-(2- hydroxyphenyl)pyridazin-4-yl)-3,6- diazabicyclo[3.2.0]heptan-6-yl)pyrimidin- 2-yl)prop-2-yn-1-yl)pyrazolo[1,5- a]pyrimidine-2-carboxamide |
| P-44 | 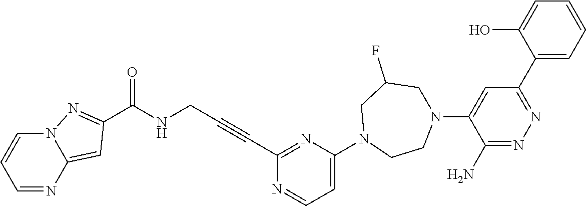 [SMILES: Nc1nnc(-c2ccccc2O)cc1N1CCN(c2ccnc(C#CCNC(=O)c3cc4ncccn4n3)n2)CC(F)C1] | N-(3-(4-(4-(3-amino-6-(2- hydroxyphenyl)pyridazin-4-yl)-6-fluoro- 1,4-diazepan-1-yl)pyrimidin-2-yl)prop-2- yn-1-yl)pyrazolo[1,5-a]pyrimidine-2- carboxamide |
| P-45 | 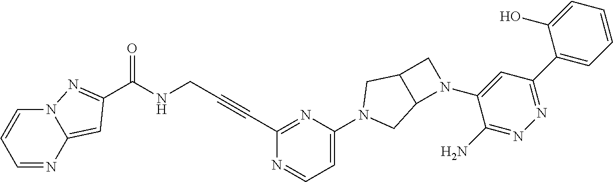 [SMILES: Nc1nnc(-c2ccccc2O)cc1N1CC2CN(c3ccnc(C#CCNC(=O)c4cc5ncccn5n4)n3)CC21] | N-(3-(4-(6-(3-amino-6-(2- hydroxyphenyl)pyridazin-4-yl)-3,6- diazabicyclo[3.2.0]heptan-3-yl)pyrimidin- 2-yl)prop-2-yn-1-yl)pyrazolo[1,5- a]pyrimidine-2-carboxamide |
| P-46 | 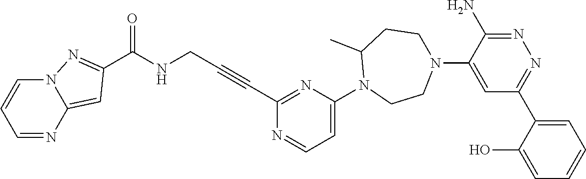 [SMILES: CC1CCN(c2cc(-c3ccccc3O)nnc2N)CCN1c1ccnc(C#CCNC(=O)c2cc3ncccn3n2)n1] | N-(3-(4-(4-(3-amino-6-(2- hydroxyphenyl)pyridazin-4-yl)-7-methyl- 1,4-diazepan-1-yl)pyrimidin-2-yl)prop-2- yn-1-yl)pyrazolo[1,5-a]pyrimidine-2- carboxamide |
| P-47 | 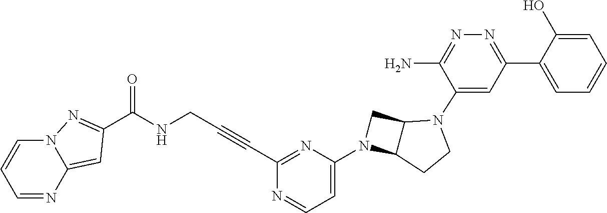 [SMILES: Nc1nnc(-c2ccccc2O)cc1N1CC[C@@H]2[C@H]1CN2c1ccnc(C#CCNC(=O)c2cc3ncccn3n2)n1] | N-(3-(4-((1R,5R)-2-(3-amino-6-(2- hydroxyphenyl)pyridazin-4-yl)-2,6- diazabicyclo[3.2.0]heptan-6-yl)pyrimidin- 2-yl)prop-2-yn-1-yl)pyrazolo[1,5- a]pyrimidine-2-carboxamide |
| P-48 | 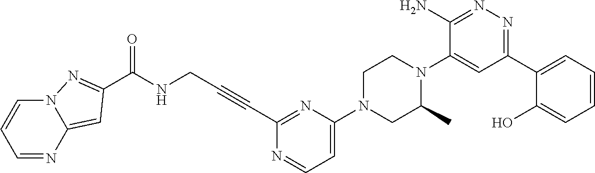 [SMILES: C[C@H]1CN(c2ccnc(C#CCNC(=O)c3cc4ncccn4n3)n2)CCN1c1cc(-c2ccccc2O)nnc1N] | (S)-N-(3-(4-(4-(3-amino-6-(2- hydroxyphenyl)pyridazin-4-yl)-3- methylpiperazin-1-yl)pyrimidin-2- yl)prop-2-yn-1-yl)pyrazolo[1,5- a]pyrimidine-2-carboxamide |
| P-49 | 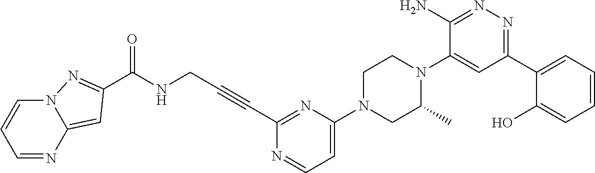 [SMILES: C[C@@H]1CN(c2ccnc(C#CCNC(=O)c3cc4ncccn4n3)n2)CCN1c1cc(-c2ccccc2O)nnc1N] | (R)-N-(3-(4-(4-(3-amino-6-(2- hydroxyphenyl)pyridazin-4-yl)-3- methylpiperazin-1-yl)pyrimidin-2- yl)prop-2-yn-1-yl)pyrazolo[1,5- a]pyrimidine-2-carboxamide |
| P-50 | 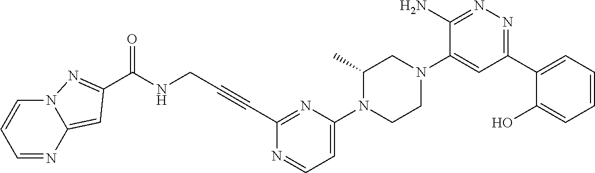 [SMILES: C[C@@H]1CN(c2cc(-c3ccccc3O)nnc2N)CCN1c1ccnc(C#CCNC(=O)c2cc3ncccn3n2)n1] | (R)-N-(3-(4-(4-(3-amino-6-(2- hydroxyphenyl)pyridazin-4-yl)-2- methylpiperazin-1-yl)pyrimidin-2- yl)prop-2-yn-1-yl)pyrazolo[1,5- a]pyrimidine-2-carboxamide |
| P-51 | 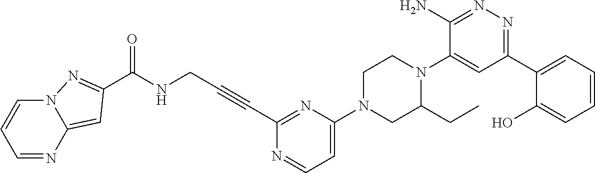 [SMILES: CCC1CN(c2ccnc(C#CCNC(=O)c3cc4ncccn4n3)n2)CCN1c1cc(-c2ccccc2O)nnc1N] | N-(3-(4-(4-(3-amino-6-(2- hydroxyphenyl)pyridazin-4-yl)-3- ethylpiperazin-1-yl)pyrimidin-2-yl)prop- 2-yn-1-yl)pyrazolo[1,5-a]pyrimidine-2- carboxamide |
| P-52 | 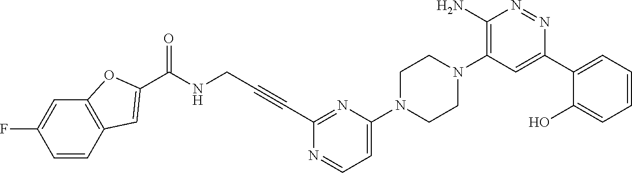 [SMILES: Nc1nnc(-c2ccccc2O)cc1N1CCN(c2ccnc(C#CCNC(=O)c3cc4ccc(F)cc4o3)n2)CC1] | N-(3-(4-(4-(3-amino-6-(2- hydroxyphenyl)pyridazin-4-yl)piperazin- 1-yl)pyrimidin-2-yl)prop-2-yn-1-yl)-6- fluorobenzofuran-2-carboxamide |
| P-53 | 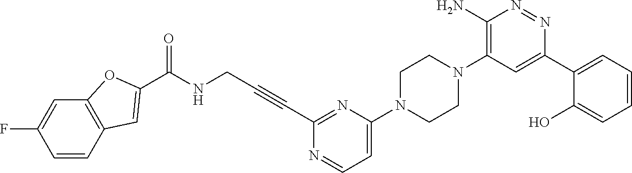 [SMILES: Nc1nnc(-c2ccccc2O)cc1N1CCN(c2ccnc(C#CCNC(=O)c3cc4ccc(F)cc4o3)n2)CC1] | N-(3-(4-(4-(3-amino-6-(2- hydroxyphenyl)pyridazin-4-yl)piperazin- 1-yl)pyrimidin-2-yl)prop-2-yn-1-yl)-6- fluorobenzofuran-2-carboxamide |
| P-54 | 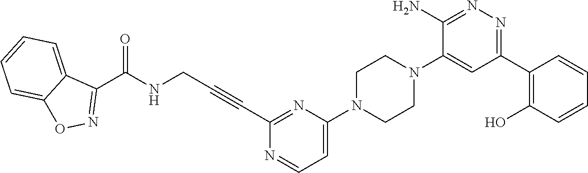 [SMILES: Nc1nnc(-c2ccccc2O)cc1N1CCN(c2ccnc(C#CCNC(=O)c3noc4ccccc34)n2)CC1] | N-(3-(4-(4-(3-amino-6-(2- hydroxyphenyl)pyridazin-4-yl)piperazin- 1-yl)pyrimidin-2-yl)prop-2-yn-1- yl)benzo[d]isoxazole-3-carboxamide |
| P-55 | 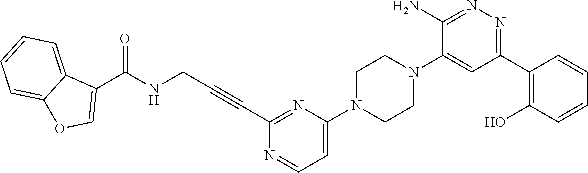 [SMILES: Nc1nnc(-c2ccccc2O)cc1N1CCN(c2ccnc(C#CCNC(=O)c3coc4ccccc34)n2)CC1] | N-(3-(4-(4-(3-amino-6-(2- hydroxyphenyl)pyridazin-4-yl)piperazin- 1-yl)pyrimidin-2-yl)prop-2-yn-1- yl)benzofuran-3-carboxamide |
| P-56 | 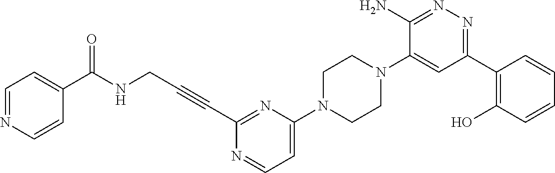 [SMILES: Nc1nnc(-c2ccccc2O)cc1N1CCN(c2ccnc(C#CCNC(=O)c3ccncc3)n2)CC1] | N-(3-(4-(4-(3-amino-6-(2- hydroxyphenyl)pyridazin-4-yl)piperazin- 1-yl)pyrimidin-2-yl)prop-2-yn-1- yl)isonicotinamide |
| P-57 | 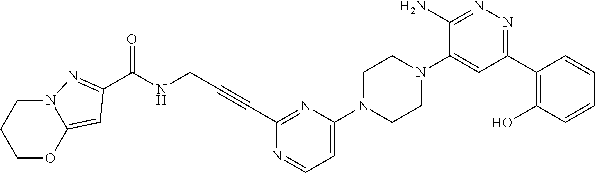 [SMILES: Nc1nnc(-c2ccccc2O)cc1N1CCN(c2ccnc(C#CCNC(=O)c3cc4n(n3)CCCO4)n2)CC1] | N-(3-(4-(4-(3-amino-6-(2- hydroxyphenyl)pyridazin-4-yl)piperazin- 1-yl)pyrimidin-2-yl)prop-2-yn-1-yl)-6,7- dihydro-5H-pyrazolo[5,1-b][1,3]oxazine- 2-carboxamide |
| P-58 | 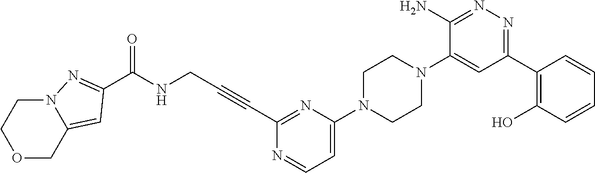 [SMILES: Nc1nnc(-c2ccccc2O)cc1N1CCN(c2ccnc(C#CCNC(=O)c3cc4n(n3)CCOC4)n2)CC1] | N-(3-(4-(4-(3-amino-6-(2- hydroxyphenyl)pyridazin-4-yl)piperazin- 1-yl)pyrimidin-2-yl)prop-2-yn-1-yl)-6,7- dihydro-4H-pyrazolo[5,1-c][1,4]oxazine- 2-carboxamide |
| P-59 | 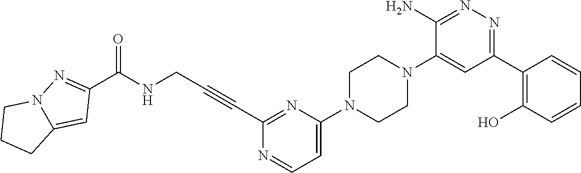 [SMILES: Nc1nnc(-c2ccccc2O)cc1N1CCN(c2ccnc(C#CCNC(=O)c3cc4n(n3)CCC4)n2)CC1] | N-(3-(4-(4-(3-amino-6-(2- hydroxyphenyl)pyridazin-4-yl)piperazin- 1-yl)pyrimidin-2-yl)prop-2-yn-1-yl)-5,6- dihydro-4H-pyrrolo[1,2-b]pyrazole-2- carboxamide |
| P-60 | 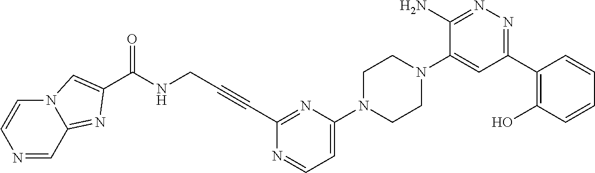 [SMILES: Nc1nnc(-c2ccccc2O)cc1N1CCN(c2ccnc(C#CCNC(=O)c3cn4ccncc4n3)n2)CC1] | N-(3-(4-(4-(3-amino-6-(2- hydroxyphenyl)pyridazin-4-yl)piperazin- 1-yl)pyrimidin-2-yl)prop-2-yn-1- yl)imidazo[1,2-a]pyrazine-2-carboxamide |
| P-61 | 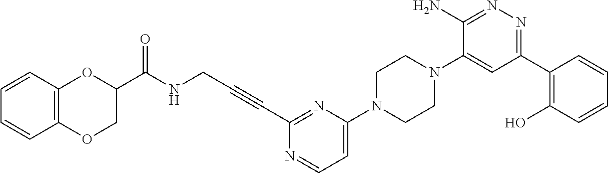 [SMILES: Nc1nnc(-c2ccccc2O)cc1N1CCN(c2ccnc(C#CCNC(=O)C3COc4ccccc4O3)n2)CC1] | N-(3-(4-(4-(3-amino-6-(2- hydroxyphenyl)pyridazin-4-yl)piperazin- 1-yl)pyrimidin-2-yl)prop-2-yn-1-yl)-2,3- dihydrobenzo[b][1,4]dioxine-2- carboxamide |
| P-62 | 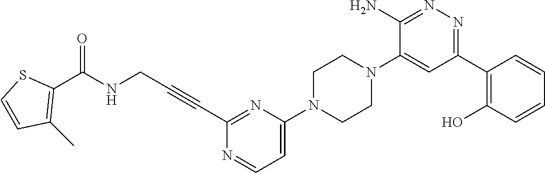 [SMILES: Cc1ccsc1C(=O)NCC#Cc1nccc(N2CCN(c3cc(-c4ccccc4O)nnc3N)CC2)n1] | N-(3-(4-(4-(3-amino-6-(2- hydroxyphenyl)pyridazin-4-yl)piperazin- 1-yl)pyrimidin-2-yl)prop-2-yn-1-yl)-4- methylthiazole-5-carboxamide |
| P-63 | 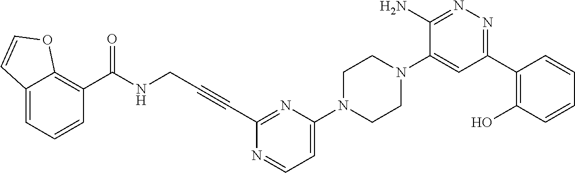 [SMILES: Nc1nnc(-c2ccccc2O)cc1N1CCN(c2ccnc(C#CCNC(=O)c3cccc4ccoc34)n2)CC1] | N-(3-(4-(4-(3-amino-6-(2- hydroxyphenyl)pyridazin-4-yl)piperazin- 1-yl)pyrimidin-2-yl)prop-2-yn-1- yl)benzofuran-7-carboxamide |
| P-64 | 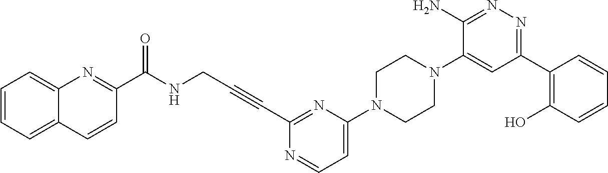 [SMILES: Nc1nnc(-c2ccccc2O)cc1N1CCN(c2ccnc(C#CCNC(=O)c3ccc4ccccc4n3)n2)CC1] | N-(3-(4-(4-(3-amino-6-(2- hydroxyphenyl)pyridazin-4-yl)piperazin- 1-yl)pyrimidin-2-yl)prop-2-yn-1- yl)quinoline-2-carboxamide |
| P-65 | 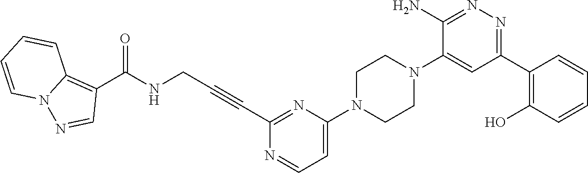 [SMILES: Nc1nnc(-c2ccccc2O)cc1N1CCN(c2ccnc(C#CCNC(=O)c3cnn4ccccc34)n2)CC1] | N-(3-(4-(4-(3-amino-6-(2- hydroxyphenyl)pyridazin-4-yl)piperazin- 1-yl)pyrimidin-2-yl)prop-2-yn-1- yl)pyrazolo[1,5-a]pyridine-3- carboxamide |
| P-66 | 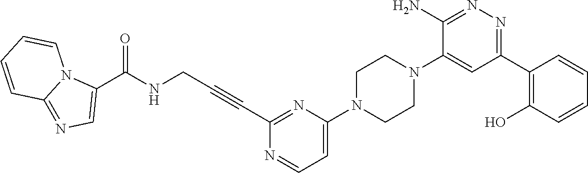 [SMILES: Nc1nnc(-c2ccccc2O)cc1N1CCN(c2ccnc(C#CCNC(=O)c3cnc4ccccn34)n2)CC1] | N-(3-(4-(4-(3-amino-6-(2- hydroxyphenyl)pyridazin-4-yl)piperazin- 1-yl)pyrimidin-2-yl)prop-2-yn-1- yl)imidazo[1,2-a]pyridine-3-carboxamide |
| P-67 | 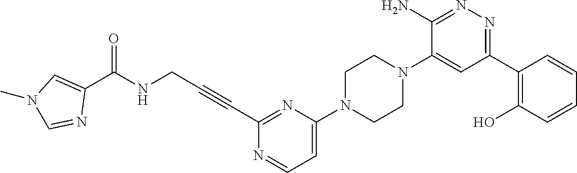 [SMILES: Cn1cnc(C(=O)NCC#Cc2nccc(N3CCN(c4cc(-c5ccccc5O)nnc4N)CC3)n2)c1] | N-(3-(4-(4-(3-amino-6-(2- hydroxyphenyl)pyridazin-4-yl)piperazin- 1-yl)pyrimidin-2-yl)prop-2-yn-1-yl)-1- methyl-1H-imidazole-4-carboxamide |
| P-68 | 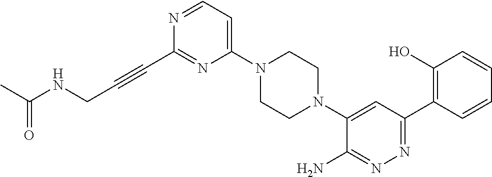 [SMILES: CC(=O)NCC#Cc1nccc(N2CCN(c3cc(-c4ccccc4O)nnc3N)CC2)n1] | N-(3-(4-(4-(3-amino-6-(2- hydroxyphenyl)pyridazin-4-yl)piperazin- 1-yl)pyrimidin-2-yl)prop-2-yn-1- yl)acetamide |
| P-69 | 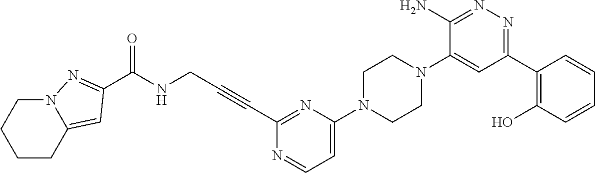 [SMILES: Nc1nnc(-c2ccccc2O)cc1N1CCN(c2ccnc(C#CCNC(=O)c3cc4n(n3)CCCC4)n2)CC1] | N-(3-(4-(4-(3-amino-6-(2- hydroxyphcnyl)pyridazin-4-yl)piperrazin- 1-yl)pyrimidin-2-yl)prop-2-yn-1-yl)- 4,5,6,7-tetrahydropyrazolo[1,5- a]pyridine-2-carboxamide |
| P-70 | 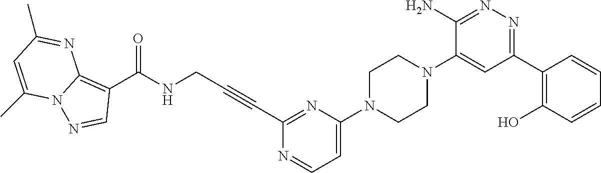 [SMILES: Cc1cc(C)n2ncc(C(=O)NCC#Cc3nccc(N4CCN(c5cc(-c6ccccc6O)nnc5N)CC4)n3)c2n1] | N-(3-(4-(4-(3-amino-6-(2- hydroxyphenyl)pyridazin-4-yl)piperazin- 1-yl)pyrimidin-2-yl)prop-2-yn-1-yl)-5,7- dimethylpyrazolo[1,5-a]pyrimidine-3- carboxamide |
| P-71 | 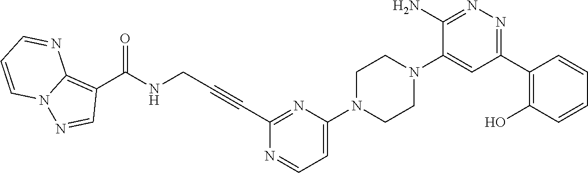 [SMILES: Nc1nnc(-c2ccccc2O)cc1N1CCN(c2ccnc(C#CCNC(=O)c3cnn4cccnc34)n2)CC1] | N-(3-(4-(4-(3-amino-6-(2- hydroxyphenyl)pyridazin-4-yl)piperazin- 1-yl)pyrimidin-2-yl)prop-2-yn-1- yl)pyrazolo[1,5-a]pyrimidine-3- carboxamide |
| P-72 | 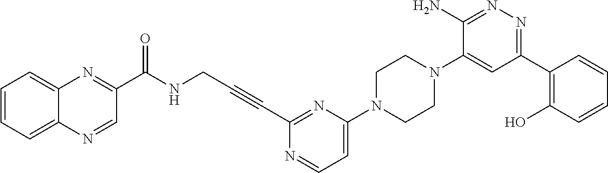 [SMILES: Nc1nnc(-c2ccccc2O)cc1N1CCN(c2ccnc(C#CCNC(=O)c3cnc4ccccc4n3)n2)CC1] | N-(3-(4-(4-(3-amino-6-(2- hydroxyphenyl)pyridazin-4-yl)piperazin- 1-yl)pyrimidin-2-yl)prop-2-yn-1- yl)quinoxaline-2-carboxamide |
| P-73 | 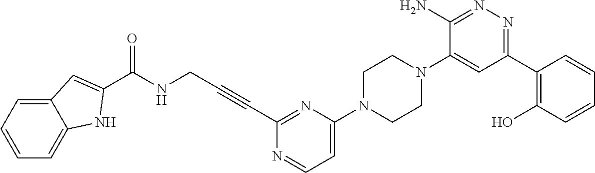 [SMILES: Nc1nnc(-c2ccccc2O)cc1N1CCN(c2ccnc(C#CCNC(=O)c3cc4ccccc4[nH]3)n2)CC1] | N-(3-(4-(4-(3-amino-6-(2- hydroxyphenyl)pyridazin-4-yl)piperazin- 1-yl)pyrimidin-2-yl)prop-2-yn-1-yl)-1H- indole-2-carboxamide |
| P-74 | 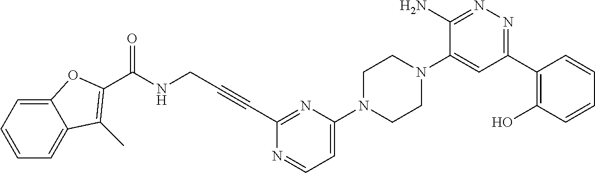 [SMILES: Cc1c(C(=O)NCC#Cc2nccc(N3CCN(c4cc(-c5ccccc5O)nnc4N)CC3)n2)oc2ccccc12] | N-(3-(4-(4-(3-amino-6-(2- hydroxyphenyl)pyridazin-4-yl)piperazin- 1-yl)pyrimidin-2-yl)prop-2-yn-1-yl)-3- methylbenzofuran-2-carboxamide |
| P-75 | 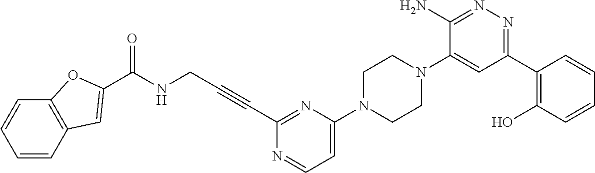 [SMILES: Nc1nnc(-c2ccccc2O)cc1N1CCN(c2ccnc(C#CCNC(=O)c3cc4ccccc4o3)n2)CC1] | N-(3-(4-(4-(3-amino-6-(2- hydroxyphenyl)pyridazin-4-yl)piperazin- 1-yl)pyrimidin-2-yl)prop-2-yn-1- yl)benzofuran-2-carboxamide |
| P-76 | 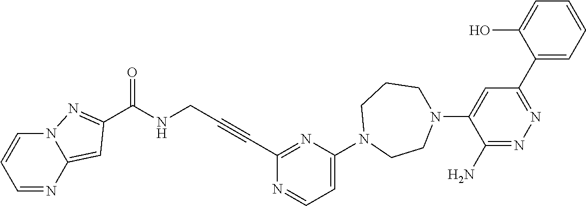 [SMILES: Nc1nnc(-c2ccccc2O)cc1N1CCCN(c2ccnc(C#CCNC(=O)c3cc4ncccn4n3)n2)CC1] | N-(3-(4-(4-(3-amino-6-(2- hydroxyphenyl)pyridazin-4-yl)-1,4- diazepan-1-yl)pyrimidin-2-yl)prop-2-yn- 1-yl)pyrazolo[1,5-a]pyrimidine-2- carboxamide |
| P-77 | 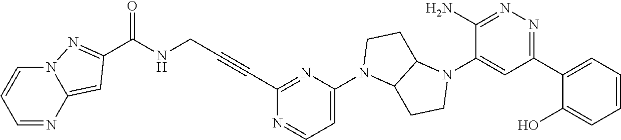 [SMILES: Nc1nnc(-c2ccccc2O)cc1N1CCC2C1CCN2c1ccnc(C#CCNC(=O)c2cc3ncccn3n2)n1] | N-(3-(4-(4-(3-amino-6-(2- hydroxyphenyl)pyridazin-4- yl)hexahydropyrrolo[3,2-b]pyrrol-1(2H)- yl)pyrimidin-2-yl)prop-2-yn-1- yl)pyrazolo[1,5-a]pyrimidine-2- carboxamide |
| P-78 | 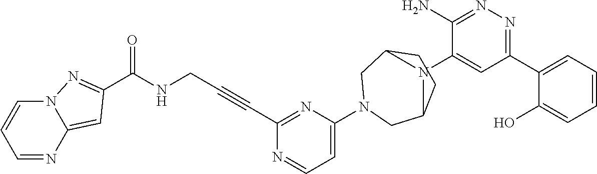 [SMILES: Nc1nnc(-c2ccccc2O)cc1N1C2CCC1CN(c1ccnc(C#CCNC(=O)c3cc4ncccn4n3)n1)C2] | N-(3-(4-(8-(3-amino-6-(2- hydroxyphenyl)pyridazin-4-yl)-3,8- diazabicyclo[3.2.1]octan-3-yl)pyrimidin- 2-yl)prop-2-yn-1-yl)pyrazolo[1,5- a]pyrimidine-2-carboxamide |
| P-79 | 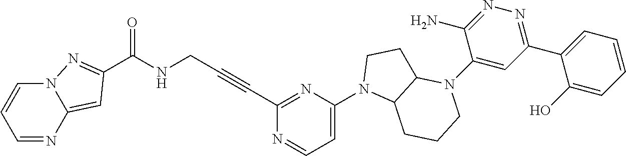 [SMILES: Nc1nnc(-c2ccccc2O)cc1N1CCCC2C1CCN2c1ccnc(C#CCNC(=O)c2cc3ncccn3n2)n1] | N-(3-(4-(4-(3-amino-6-(2- hydroxyphenyl)pyridazin-4-yl)octahydro- 1H-pyrrolo[3,2-b]pyridin-1-yl)pyrimidin- 2-yl)prop-2-yn-1-yl)pyrazolo[1,5- a]pyrimidine-2-carboxamide |
| P-80 | 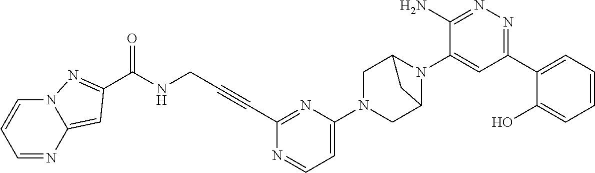 [SMILES: Nc1nnc(-c2ccccc2O)cc1N1C2CC1CN(c1ccnc(C#CCNC(=O)c3cc4ncccn4n3)n1)C2] | N-(3-(4-(6-(3-amino-6-(2- hydroxyphenyl)pyridazin-4-yl)-3,6- diazabicyclo[3.1.1]heptan-3-yl)pyrimidin- 2-yl)prop-2-yn-1-yl)pyrazolo[1,5- a]pyrimidine-2-carboxamide |
| P-81 | 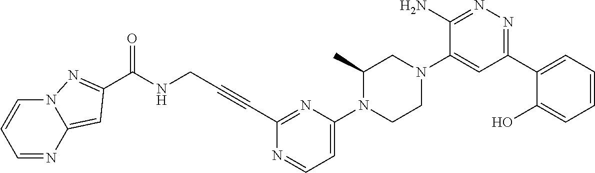 [SMILES: C[C@H]1CN(c2cc(-c3ccccc3O)nnc2N)CCN1c1ccnc(C#CCNC(=O)c2cc3ncccn3n2)n1] | (R)-N-(3-(4-(4-(3-amino-6-(2- hydroxyphenyl)pyridazin-4-yl)-2- methylpiperazin-1-yl)pyrimidin-2- yl)prop-2-yn-1-yl)pyrazolo[1,5- a]pyrimidine-2-carboxamide |
| P-82 | 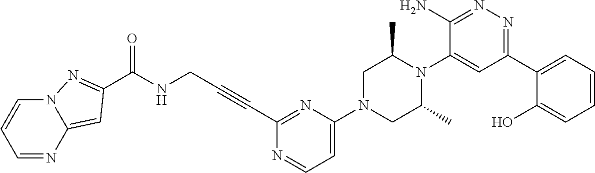 [SMILES: C[C@@H]1CN(c2ccnc(C#CCNC(=O)c3cc4ncccn4n3)n2)C[C@@H](C)N1c1cc(-c2ccccc2O)nnc1N] | N-(3-(4-((3R,5R)-4-(3-amino-6-(2- hydroxyphenyl)pyridazin-4-yl)-3,5- dimethylpiperazin-1-yl)pyrimidin-2- yl)prop-2-yn-1-yl)pyrazolo[1,5- a]pyrimidine-2-carboxamide |
| P-83 | 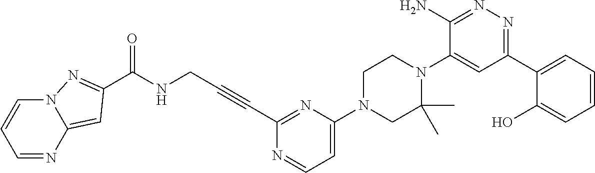 [SMILES: CC1(C)CN(c2ccnc(C#CCNC(=O)c3cc4ncccn4n3)n2)CCN1c1cc(-c2ccccc2O)nnc1N] | N-(3-(4-(4-(3-amino-6-(2- hydroxyphenyl)pyridazin-4-yl)-3,3- dimethylpiperazin-1-yl)pyrimidin-2- yl)prop-2-yn-1-yl)pyrazolo[1,5- a]pyrimidine-2-carboxamide |
| P-84 | 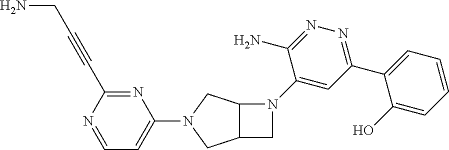 [SMILES: NCC#Cc1nccc(N2CC3CN(c4cc(-c5ccccc5O)nnc4N)C3C2)n1] | 2-(6-amino-5-(3-(2-(3-aminoprop-1-yn-1- yl)pyrimidin-4-yl)-3,6- diazabicyclo[3.2.0]heptan-6-yl)pyridazin- 3-yl)phenol |
| P-85 | 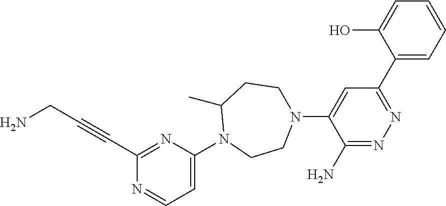 [SMILES: CC1CCN(c2cc(-c3ccccc3O)nnc2N)CCN1c1ccnc(C#CCN)n1] | 2-(6-amino-5-(4-(2-(3-aminoprop-1-yn-1- yl)pyrimidin-4-yl)-5-methyl-1,4- diazepan-1-yl)pyridazin-3-yl)phenol |
| P-86 | 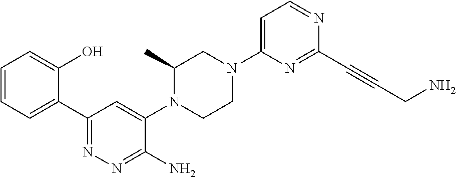 [SMILES: C[C@H]1CN(c2ccnc(C#CCN)n2)CCN1c1cc(-c2ccccc2O)nnc1N] | (S)-2-(6-amino-5-(4-(2-(3-aminoprop-1- yn-1-yl)pyrimidin-4-yl)-2- methylpiperazin-1-yl)pyridazin-3- yl)phenol |
| P-87 | 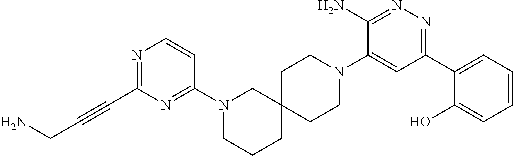 [SMILES: NCC#Cc1nccc(N2CCCC3(CCN(c4cc(-c5ccccc5O)nnc4N)CC3)C2)n1] | 2-(6-amino-5-(2-(2-(3-aminoprop-1-yn-1- yl)pyrimidin-4-yl)-2,9- diazaspiro[5.5]undecan-9-yl)pyridazin-3- yl)phenol |
| P-88 | 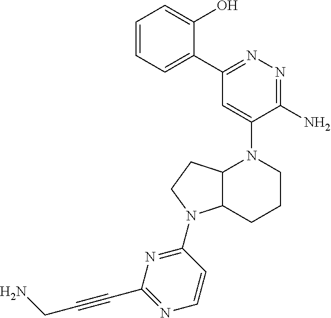 [SMILES: NCC#Cc1nccc(N2CCC3C2CCCN3c2cc(-c3ccccc3O)nnc2N)n1] | 2-(6-amino-5-(1-(2-(3-aminoprop-1-yn-1- yl)pyrimidin-4-yl)octahydro-4H- pyrrolo[3,2-b]pyridin-4-yl)pyridazin-3- yl)phenol |
| P-89 | 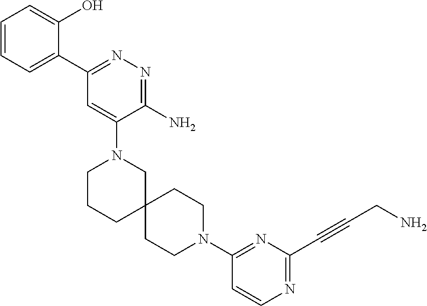 [SMILES: NCC#Cc1nccc(N2CCC3(CCCN(c4cc(-c5ccccc5O)nnc4N)C3)CC2)n1] | 2-(6-amino-5-(9-(2-(3-aminoprop-1-yn-1- yl)pyrimidin-4-yl)-2,9- diazaspiro[5.5]undecan-2-yl)pyridazin-3- yl)phenol |
| P-90 | 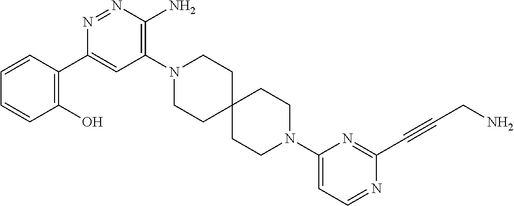 [SMILES: NCC#Cc1nccc(N2CCC3(CC2)CCN(c2cc(-c4ccccc4O)nnc2N)CC3)n1] | 2-(6-amino-5-(9-(2-(3-aminoprop-1-yn-1- yl)pyrimidin-4-yl)-3,9- diazaspiro[5.5]undecan-3-yl)pyridazin-3- yl)phenol |
| P-91 | 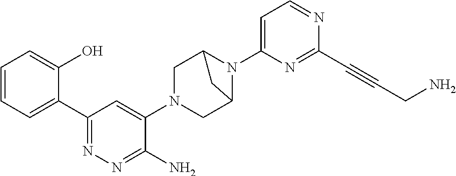 [SMILES: NCC#Cc1nccc(N2C3CC2CN(c2cc(-c4ccccc4O)nnc2N)C3)n1] | 2-(6-amino-5-(6-(2-(3-aminoprop-1-yn-1- yl)pyrimidin-4-yl)-3,6- diazabicyclo[3.1.1]heptan-3-yl)pyridazin- 3-yl)phenol |
| P-92 | 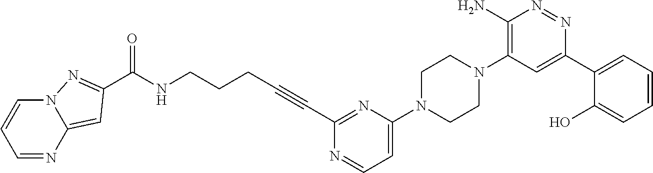 [SMILES: Nc1nnc(-c2ccccc2O)cc1N1CCN(c2ccnc(C#CCCCNC(=O)c3cc4ncccn4n3)n2)CC1] | N-(5-(4-(4-(3-amino-6-(2- hydroxyphenyl)pyridazin-4-yl)piperazin- 1-yl)pyrimidin-2-yl)pent-4-yn-1- yl)pyrazolo[1,5-a]pyrimidine-2- carboxamide |
| P-93 | 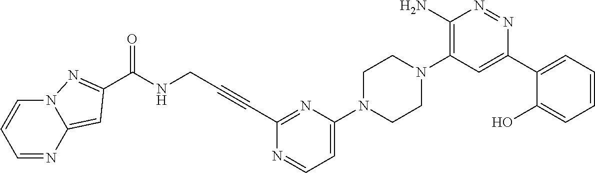 [SMILES: Nc1nnc(-c2ccccc2O)cc1N1CCN(c2ccnc(C#CCNC(=O)c3cc4ncccn4n3)n2)CC1] | N-(3-(4-(4-(3-amino-6-(2- hydroxyphenyl)pyridazin-4-yl)piperazin- 1-yl)pyrimidin-2-yl)prop-2-yn-1- yl)pyrazolo[1,5-a]pyrimidine-2- carboxamide |
| P-94 | 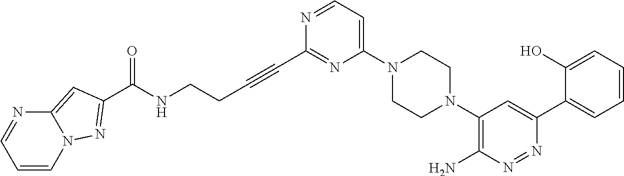 [SMILES: Nc1nnc(-c2ccccc2O)cc1N1CCN(c2ccnc(C#CCCNC(=O)c3cc4ncccn4n3)n2)CC1] | N-(4-(4-(4-(3-amino-6-(2- hydroxyphenyl)pyridazin-4-yl)piperazin- 1-yl)pyrimidin-2-yl)but-3-yn-1- yl)pyrazolo[1,5-a]pyrimidine-2- carboxamide |
| P-95 | 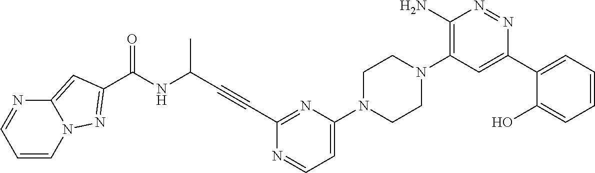 [SMILES: CC(C#Cc1nccc(N2CCN(c3cc(-c4ccccc4O)nnc3N)CC2)n1)NC(=O)c1cc2ncccn2n1] | N-(4-(4-(4-(3-amino-6-(2- hydroxyphenyl)pyridazin-4-yl)piperazin- 1-yl)pyrimidin-2-yl)but-3-yn-2- yl)pyrazolo[1,5-a]pyrimidine-2- carboxamide |
| P-96 | 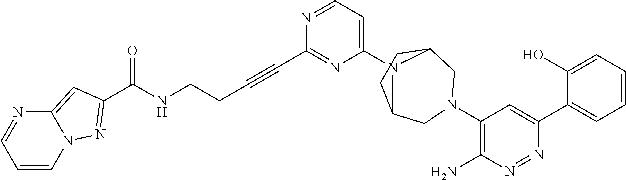 [SMILES: Nc1nnc(-c2ccccc2O)cc1N1CC2CCC(C1)N2c1ccnc(C#CCCNC(=O)c2cc3ncccn3n2)n1] | N-(4-(4-(3-(3-amino-6-(2- hydroxyphenyl)pyridazin-4-yl)-3,8- diazabicyclo[3.2.1]octan-8-yl)pyrimidin- 2-yl)but-3-yn-1-yl)pyrazolo[1,5- a]pyrimidine-2-carboxamide |
| P-97 | 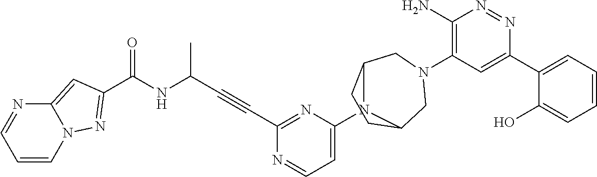 [SMILES: CC(C#Cc1nccc(N2C3CCC2CN(c2cc(-c4ccccc4O)nnc2N)C3)n1)NC(=O)c1cc2ncccn2n1] | N-(4-(4-(3-(3-amino-6-(2- hydroxyphenyl)pyridazin-4-yl)-3,8- diazabicyclo[3.2.1]octan-8-yl)pyrimidin- 2-yl)but-3-yn-2-yl)pyrazolo[1,5- a]pyrimidine-2-carboxamide |
| P-98 | 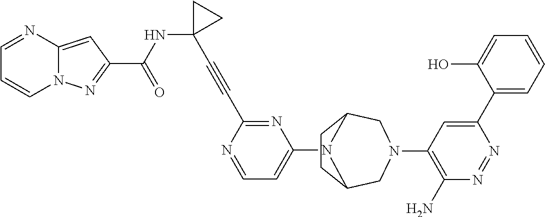 [SMILES: Nc1nnc(-c2ccccc2O)cc1N1CC2CCC(C1)N2c1ccnc(C#CC2(NC(=O)c3cc4ncccn4n3)CC2)n1] | N-(1-((4-(3-(3-amino-6-(2- hydroxyphenyl)pyridazin-4-yl)-3,8- diazabicyclo[3.2.1]octan-8-yl)pyrimidin- 2-yl)ethynyl)cyclopropyl)pyrazolo[1,5- a]pyrimidine-2-carboxamide |
| P-99 | 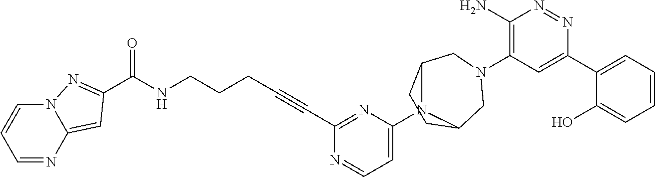 [SMILES: Nc1nnc(-c2ccccc2O)cc1N1CC2CCC(C1)N2c1ccnc(C#CCCCNC(=O)c2cc3ncccn3n2)n1] | N-(5-(4-(3-(3-amino-6-(2- hydroxyphenyl)pyridazin-4-yl)-3,8- diazabicyclo[3.2.1]octan-8-yl)pyrimidin- 2-yl)pent-4-yn-1-yl)pyrazolo[1,5- a]pyrimidine-2-carboxamide |
| P-100 | 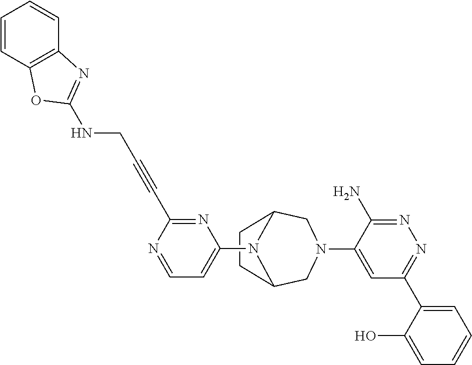 [SMILES: Nc1nnc(-c2ccccc2O)cc1N1CC2CCC(C1)N2c1ccnc(C#CCNc2nc3ccccc3o2)n1] | 2-(6-amino-5-(8-(2-(3-(benzo[d]oxazol-2- ylamino)prop-1-yn-1-yl)pyrimidin-4-yl)- 3,8-diazabicyclo[3.2.1]octan-3- yl)pyridazin-3-yl)phenol |
| P-101 | 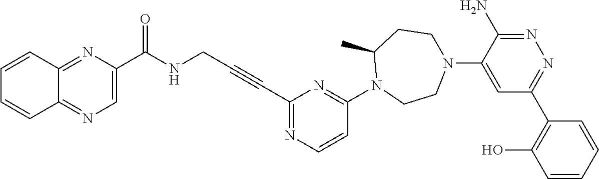 [SMILES: C[C@H]1CCN(c2cc(-c3ccccc3O)nnc2N)CCN1c1ccnc(C#CCNC(=O)c2cnc3ccccc3n2)n1] | (S)-N-(3-(4-(4-(3-amino-6-(2- hydroxyphenyl)pyridazin-4-yl)-7-methyl- 1,4-diazepan-1-yl)pyrimidin-2-yl)prop-2- yn-1-yl)quinoxaline-2-carboxamide |
| P-102 | 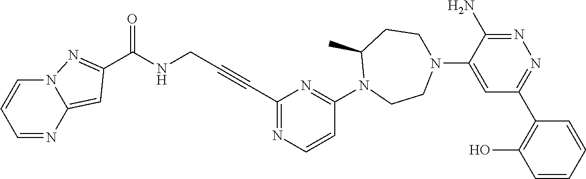 [SMILES: C[C@H]1CCN(c2cc(-c3ccccc3O)nnc2N)CCN1c1ccnc(C#CCNC(=O)c2cc3ncccn3n2)n1] | (S)-N-(3-(4-(4-(3-amino-6-(2- hydroxyphenyl)pyridazin-4-yl)-7-methyl- 1,4-diazepan-1-yl)pyrimidin-2-yl)prop-2- yn-1-yl)pyrazolo[1,5-a]pyrimidine-2- carboxamide |
| P-103 | 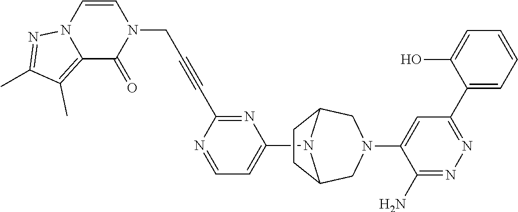 [SMILES: Cc1nn2ccn(CC#Cc3nccc(N4C5CCC4CN(c4cc(-c6ccccc6O)nnc4N)C5)n3)c(=O)c2c1C] | 5-(3-(4-(3-(3-amino-6-(2- hydroxyphenyl)pyridazin-4-yl)-3,8- diazabicyclo[3.2.1]octan-8-yl)pyrimidin- 2-yl)prop-2-yn-1-yl)-2,3- dimethylpyrazolo[1,5-a]pyrazin-4(5H)- one |
| P-104 | 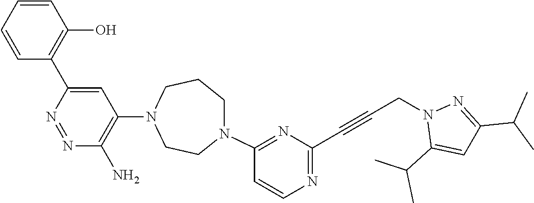 [SMILES: CC(C)c1cc(C(C)C)n(CC#Cc2nccc(N3CCCN(c4cc(-c5ccccc5O)nnc4N)CC3)n2)n1] | 2-(6-amino-5-(4-(2-(3-(3,5-diisopropyl- 1H-pyrazol-1-yl)prop-1-yn-1- yl)pyrimidin-4-yl)-1,4-diazepan-1- yl)pyridazin-3-yl)phenol |
| P-105 | 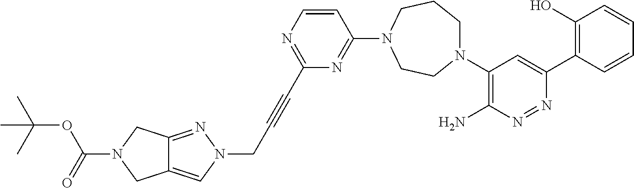 [SMILES: CC(C)(C)OC(=O)N1Cc2cn(CC#Cc3nccc(N4CCCN(c5cc(-c6ccccc6O)nnc5N)CC4)n3)nc2C1] | tert-butyl 2-(3-(4-(4-(3-amino-6-(2- hydroxyphenyl)pyridazin-4-yl)-1,4- diazepan-1-yl)pyrimidin-2-yl)prop-2-yn- 1-yl)-2,6-dihydropyrrolo[3,4-c]pyrazole- 5(4H)-carboxylate |
| P-106 | 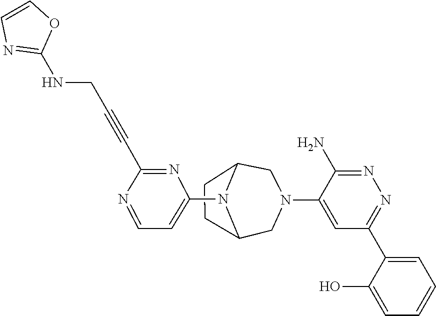 [SMILES: Nc1nnc(-c2ccccc2O)cc1N1CC2CCC(C1)N2c1ccnc(C#CCNc2ncco2)n1] | 2-(6-amino-5-(8-(2-(3-(oxazol-2- ylamino)prop-1-yn-1-yl)pyrimidin-4-yl)- 3,8-diazabicyclo[3.2.1]octan-3- yl)pyridazin-3-yl)phenol |
| P-107 | 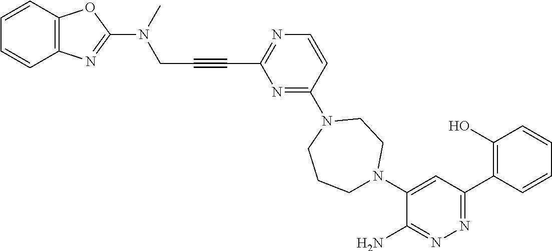 [SMILES: CN(CC#Cc1nccc(N2CCCN(c3cc(-c4ccccc4O)nnc3N)CC2)n1)c1nc2ccccc2o1] | 2-(6-amino-5-(4-(2-(3-(benzo[d]oxazol-2- yl(methyl)amino)prop-1-yn-1- yl)pyrimidin-4-yl)-1,4-diazepan-1- yl)pyridazin-3-yl)phenol |
| P-108 | 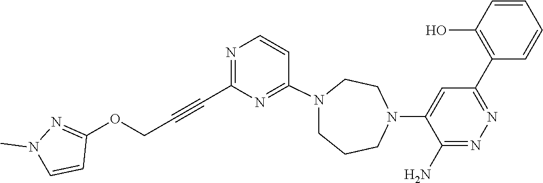 [SMILES: Cn1ccc(OCC#Cc2nccc(N3CCCN(c4cc(-c5ccccc5O)nnc4N)CC3)n2)n1] | 2-(6-amino-5-(4-(2-(3-((1-methyl-1H- pyrazol-3-yl)oxy)prop-1-yn-1- yl)pyrimidin-4-yl)-1,4-diazepan-1- yl)pyridazin-3-yl)phenol |
| P-109 | 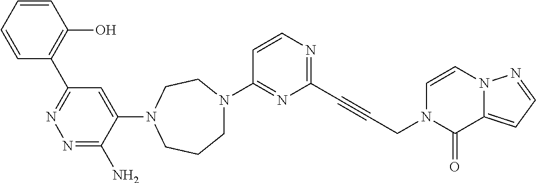 | 5-(3-(4-(4-(3-amino-6-(2- hydroxyphenyl)pyridazin-4-yl)-1,4- diazepan-1-yl)pyrimidin-2-yl)prop-2-yn- 1-yl)pyrazolo[1,5-a]pyrazin-4(5H)-one |
| P-110 | 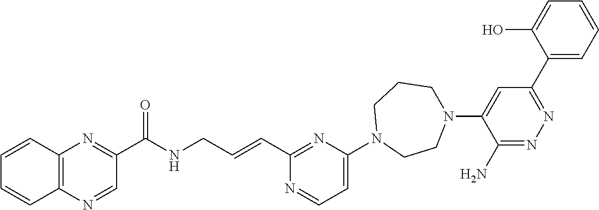 [SMILES: Nc1nnc(-c2ccccc2O)cc1N1CCCN(c2ccnc(/C=C/CNC(=O)c3cnc4ccccc4n3)n2)CC1] | (E)-N-(3-(4-(4-(3-amino-6-(2- hydroxyphenyl)pyridazin-4-yl)-1,4- diazepan-1-yl)pyrimidin-2- yl)allyl)quinoxaline-2-carboxamide |
| P-111 | 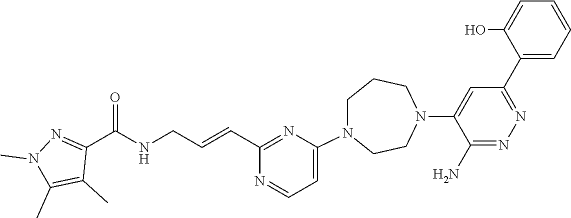 [SMILES: Cc1c(C(=O)NC/C=C/c2nccc(N3CCCN(c4cc(-c5ccccc5O)nnc4N)CC3)n2)nn(C)c1C] | (E)-N-(3-(4-(4-(3-amino-6-(2- hydroxyphenyl)pyridazin-4-yl)-1,4- diazepan-1-yl)pyrimidin-2-yl)allyl)-1,4,5- trimethyl-1H-pyrazole-3-carboxamide |
| P-112 | 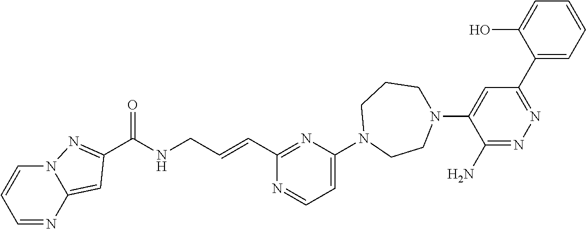 [SMILES: Nc1nnc(-c2ccccc2O)cc1N1CCCN(c2ccnc(/C=C/CNC(=O)c3cc4ncccn4n3)n2)CC1] | (E)-N-(3-(4-(4-(3-amino-6-(2- hydroxyphenyl)pyridazin-4-yl)-1,4- diazepan-1-yl)pyrimidin-2- yl)allyl)pyrazolo[1,5-a]pyrimidine-2- carboxamide |
| P-113 | 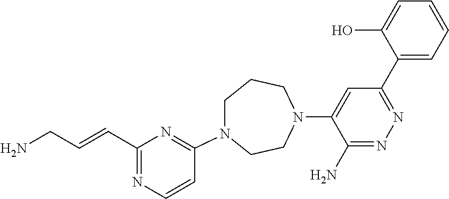 [SMILES: NC/C=C/c1nccc(N2CCCN(c3cc(-c4ccccc4O)nnc3N)CC2)n1] | (E)-2-(6-amino-5-(4-(2-(3-aminoprop-1- en-1-yl)pyrimidin-4-yl)-1,4-diazepan-1- yl)pyridazin-3-yl)phenol |
| P-114 | 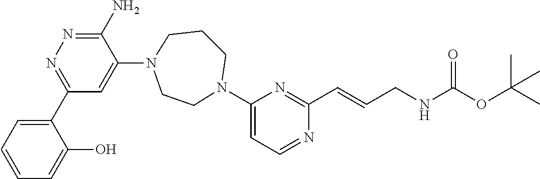 [SMILES: CC(C)(C)OC(=O)NC/C=C/c1nccc(N2CCCN(c3cc(-c4ccccc4O)nnc3N)CC2)n1] | tert-butyl (E)-(3-(4-(4-(3-amino-6-(2- hydroxyphenyl)pyridazin-4-yl)-1,4- diazepan-1-yl)pyrimidin-2- yl)allyl)carbamate |
| P-115 | 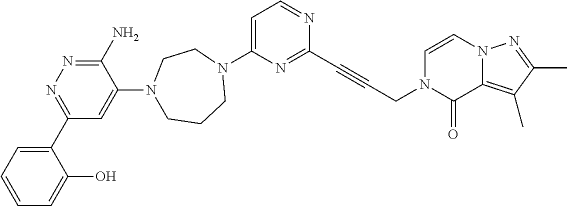 [SMILES: Cc1nn2ccn(CC#Cc3nccc(N4CCCN(c5cc(-c6ccccc6O)nnc5N)CC4)n3)c(=O)c2c1C] | 5-(3-(4-(4-(3-amino-6-(2- hydroxyphenyl)pyridazin-4-yl)-1,4- diazepan-1-yl)pyrimidin-2-yl)prop-2-yn- 1-yl)-2,3-dimethylpyrazolo[1,5-a]pyrazin- 4(5H)-one |
| P-116 | 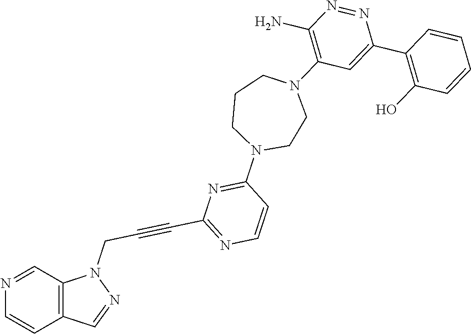 [SMILES: Nc1nnc(-c2ccccc2O)cc1N1CCCN(c2ccnc(C#CCn3ncc4ccncc43)n2)CC1] | 2-(5-(4-(2-(3-(1H-pyrazolo[3,4-c]pyridin- 1-yl)prop-1-yn-1-yl)pyrimidin-4-yl)-1,4- diazepan-1-yl)-6-aminopyridazin-3- yl)phenol |
| P-117 | 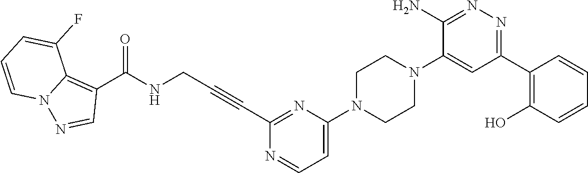 [SMILES: Nc1nnc(-c2ccccc2O)cc1N1CCN(c2ccnc(C#CCNC(=O)c3cnn4cccc(F)c34)n2)CC1] | N-(3-(4-(4-(3-amino-6-(2- hydroxyphenyl)pyridazin-4-yl)piperazin- 1-yl)pyrimidin-2-yl)prop-2-yn-1-yl)-4- fluoropyrazolo[1,5-a]pyridine-3- carboxamide |
| P-118 | 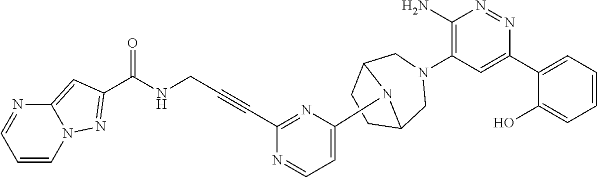 [SMILES: Nc1nnc(-c2ccccc2O)cc1N1CC2CCC(C1)N2c1ccnc(C#CCNC(=O)c2cc3ncccn3n2)n1] | N-(3-(4-(4-(3-amino-6-(2- hydroxyphenyl)pyridazin-4-yl)piperazin- 1-yl)pyrimidin-2-yl)prop-2-yn-1- yl)benzo[d]thiazole-2-carboxamide |
| P-119 | 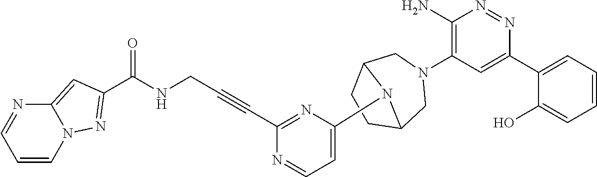 [SMILES: Nc1nnc(-c2ccccc2O)cc1N1CC2CCC(C1)N2c1ccnc(C#CCNC(=O)c2cc3ncccn3n2)n1] | N-(3-(4-(3-(3-amino-6-(2- hydroxyphenyl)pyridazin-4-yl)-3,8- diazabicyclo[3.2.1]octan-8-yl)pyrimidin- 2-yl)prop-2-yn-1-yl)pyrazolo[1,5- a]pyrimidine-2-carboxamide |
| P-120 | 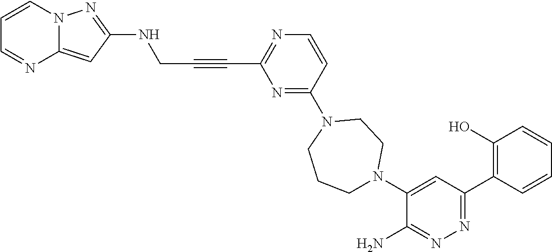 [SMILES: Nc1nnc(-c2ccccc2O)cc1N1CCCN(c2ccnc(C#CCNc3cc4ncccn4n3)n2)CC1] | 2-(6-amino-5-(4-(2-(3-(pyrazolo[1,5- a]pyrimidin-2-ylamino)prop-1-yn-1- yl)pyrimidin-4-yl)-1,4-diazepan-1- yl)pyridazin-3-yl)phenol |
| P-121 | 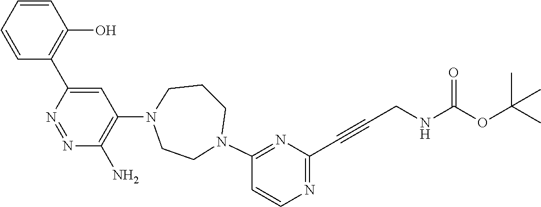 [SMILES: CC(C)(C)OC(=O)NCC#Cc1nccc(N2CCCN(c3cc(-c4ccccc4O)nnc3N)CC2)n1] | tert-butyl (3-(4-(4-(3-amino-6-(2- hydroxyphenyl)pyridazin-4-yl)-1,4- diazepan-1-yl)pyrimidin-2-yl)prop-2-yn- 1-yl)carbamate |
| P-122 | 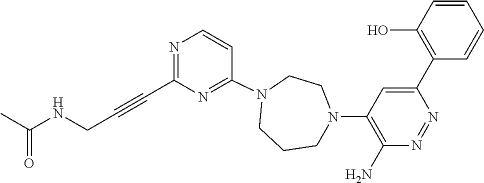 [SMILES: CC(=O)NCC#Cc1nccc(N2CCCN(c3cc(-c4ccccc4O)nnc3N)CC2)n1] | N-(3-(4-(4-(3-amino-6-(2- hydroxyphenyl)pyridazin-4-yl)-1,4- diazepan-1-yl)pyrimidin-2-yl)prop-2-yn- 1-yl)acetamide |
| P-123 | 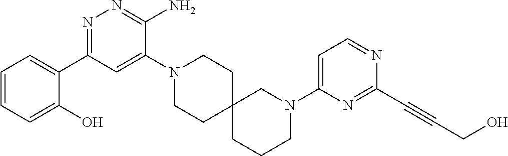 [SMILES: Nc1nnc(-c2ccccc2O)cc1N1CCC2(CCCN(c3ccnc(C#CCO)n3)C2)CC1] | 2-(6-amino-5-(2-(2-(3-hydroxyprop-1-yn- 1-yl)pyrimidin-4-yl)-2,9- diazaspiro[5.5]undecan-9-yl)pyridazin-3- yl)phenol |
| P-124 | 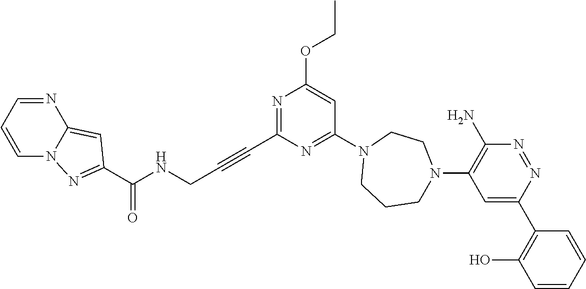 [SMILES: CCOc1cc(N2CCCN(c3cc(-c4ccccc4O)nnc3N)CC2)nc(C#CCNC(=O)c2cc3ncccn3n2)n1] | N-(3-(4-(4-(3-amino-6-(2- hydroxyphenyl)pyridazin-4-yl)-1,4- diazepan-1-yl)-6-ethoxypyrimidin-2- yl)prop-2-yn-1-yl)pyrazolo[1,5- a]pyrimidine-2-carboxamide |
| P-125 | 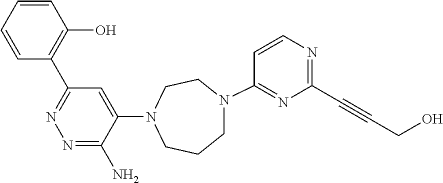 [SMILES: Nc1nnc(-c2ccccc2O)cc1N1CCCN(c2ccnc(C#CCO)n2)CC1] | 2-(6-amino-5-(4-(2-(3-hydroxyprop-1-yn- 1-yl)pyrimidin-4-yl)-1,4-diazepan-1- yl)pyridazin-3-yl)phenol |
| P-126 | 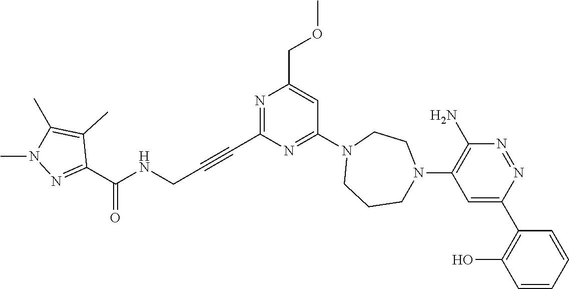 [SMILES: COCc1cc(N2CCCN(c3cc(-c4ccccc4O)nnc3N)CC2)nc(C#CCNC(=O)c2nn(C)c(C)c2C)n1] | N-(3-(4-(4-(3-amino-6-(2- hydroxyphenyl)pyridazin-4-yl)-1,4- diazepan-1-yl)-6- (methoxymethyl)pyrimidin-2-yl)prop-2- yn-1-yl)-1,4,5-trimethyl-1H-pyrazole-3- carboxamide |
| P-127 | 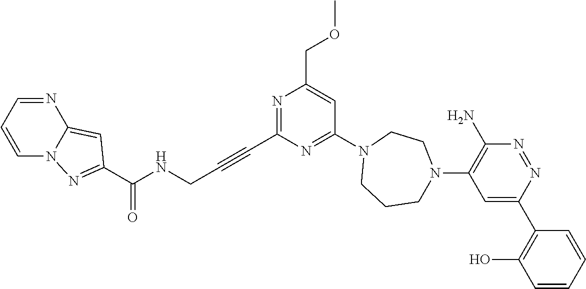 [SMILES: COCc1cc(N2CCCN(c3cc(-c4ccccc4O)nnc3N)CC2)nc(C#CCNC(=O)c2cc3ncccn3n2)n1] | N-(3-(4-(4-(3-amino-6-(2- hydroxyphenyl)pyridazin-4-yl)-1,4- diazepan-1-yl)-6- (methoxymethyl)pyrimidin-2-yl)prop-2- yn-1-yl)pyrazolo\|1,5-a\|pyrimidine-2- carboxamide |
| P-128 | 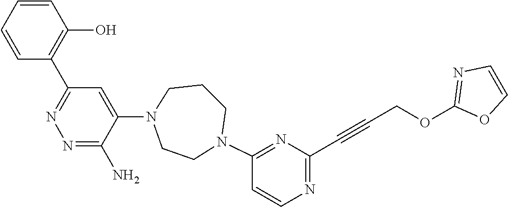 [SMILES: Nc1nnc(-c2ccccc2O)cc1N1CCCN(c2ccnc(C#CCOc3ncco3)n2)CC1] | 2-(6-amino-5-(4-(2-(3-(oxazol-2- yloxy)prop-1-yn-1-yl)pyrimidin-4-yl)- 1,4-diazepan-1-yl)pyridazin-3-yl)phenol |
| P-129 | 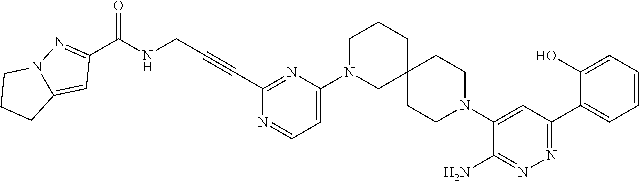 [SMILES: Nc1nnc(-c2ccccc2O)cc1N1CCC2(CCCN(c3ccnc(C#CCNC(=O)c4cc5n(n4)CCC5)n3)C2)CC1] | N-(3-(4-(9-(3-amino-6-(2- hydroxyphenyl)pyridazin-4-yl)-2,9- diazaspiro[5.5]undecan-2-yl)pyrimidin-2- yl)prop-2-yn-1-yl)-5,6-dihydro-4H- pyrrolo[1,2-b]pyrazole-2-carboxamide |
| P-130 | 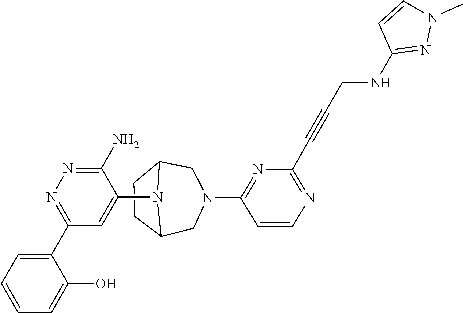 [SMILES: Cn1ccc(NCC#Cc2nccc(N3CC4CCC(C3)N4c3cc(-c4ccccc4O)nnc3N)n2)n1] | 2-(6-amino-5-(3-(2-(3-((1-methyl-1H- pyrazol-3-yl)amino)prop-1-yn-1- yl)pyrimidin-4-yl)-3,8- diazabicyclo[3.2.1]octan-8-yl)pyridazin- 3-yl)phenol |
| P-131 | 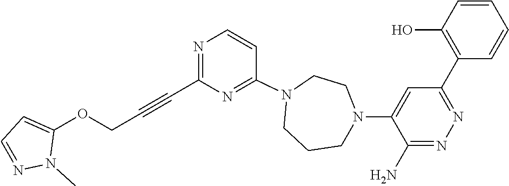 [SMILES: Cn1nccc1OCC#Cc1nccc(N2CCCN(c3cc(-c4ccccc4O)nnc3N)CC2)n1] | 2-(6-amino-5-(4-(2-(3-((1-methyl-1H- pyrazol-5-yl)oxy)prop-1-yn-1- yl)pyrimidin-4-yl)-1,4-diazepan-1- yl)pyridazin-3-yl)phenol |
| P-132 | 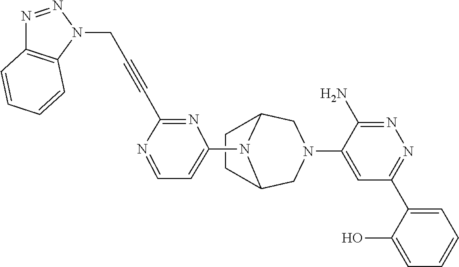 [SMILES: Nc1nnc(-c2ccccc2O)cc1N1CC2CCC(C1)N2c1ccnc(C#CCn2nnc3ccccc32)n1] | 2-(5-(8-(2-(3-(1H-benzo[d][1,2,3]triazol- 1-yl)prop-1-yn-1-yl)pyrimidin-4-yl)-3,8- diazabicyclo[3.2.1]octan-3-yl)-6- aminopyridazin-3-yl)phenol |
| P-133 | 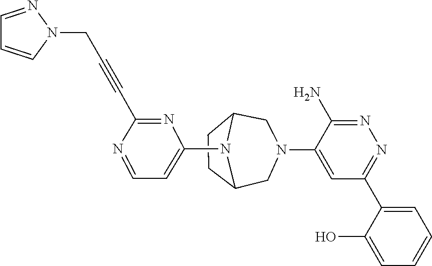 [SMILES: Nc1nnc(-c2ccccc2O)cc1N1CC2CCC(C1)N2c1ccnc(C#CCn2cccn2)n1] | 2-(5-(8-(2-(3-(1H-pyrazol-1-yl)prop-1- yn-1-yl)pyrimidin-4-yl)-3,8- diazabicyclo[3.2.1]octan-3-yl)-6- aminopyridazin-3-yl)phenol |
| P-134 | 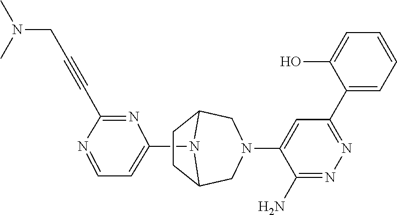 [SMILES: CN(C)CC#Cc1nccc(N2C3CCC2CN(c2cc(-c4ccccc4O)nnc2N)C3)n1] | 2-(6-amino-5-(8-(2-(3- (dimethylamino)prop-1-yn-1- yl)pyrimidin-4-yl)-3,8- diazabicyclo[3.2.1]octan-3-yl)pyridazin- 3-yl)phenol |
| P-135 | 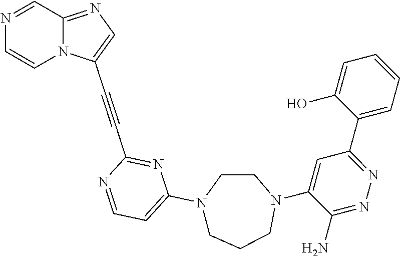 [SMILES: Nc1nnc(-c2ccccc2O)cc1N1CCCN(c2ccnc(C#Cc3cnc4cnccn34)n2)CC1] | 2-(6-amino-5-(4-(2-(imidazo[1,2- a]pyrazin-3-ylethynyl)pyrimidin-4-yl)- 1,4-diazepan-1-yl)pyridazin-3-yl)phenol |
| P-136 | 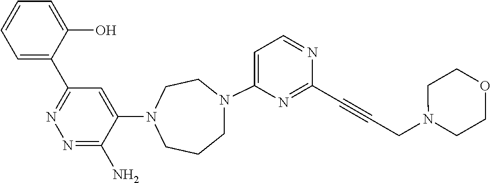 [SMILES: Nc1nnc(-c2ccccc2O)cc1N1CCCN(c2ccnc(C#CCN3CCOCC3)n2)CC1] | 2-(6-amino-5-(4-(2-(3-morpholinoprop-1- yn-1-yl)pyrimidin-4-yl)-1,4-diazepan-1- yl)pyridazin-3-yl)phenol |
| P-137 | 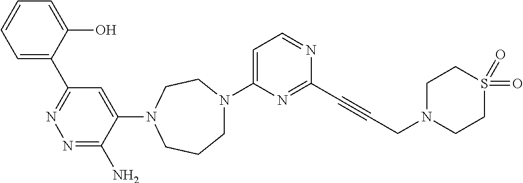 | 4-(3-(4-(4-(3-amino-6-(2- hydroxyphenyl)pyridazin-4-yl)-1,4- diazepan-1-yl)pyrimidin-2-yl)prop-2-yn- 1-yl)thiomorpholine 1,1-dioxide |
| P-138 | 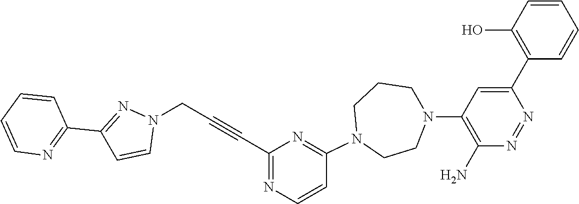 [SMILES: Nc1nnc(-c2ccccc2O)cc1N1CCCN(c2ccnc(C#CCn3ccc(-c4ccccn4)n3)n2)CC1] | 2-(6-amino-5-(4-(2-(3-(3-(pyridin-2-yl)- 1H-pyrazol-1-yl)prop-1-yn-1- yl)pyrimidin-4-yl)-1,4-diazepan-1- yl)pyridazin-3-yl)phenol |
| P-139 | 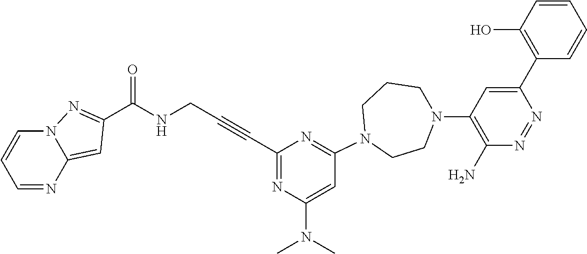 [SMILES: CN(C)c1cc(N2CCCN(c3cc(-c4ccccc4O)nnc3N)CC2)nc(C#CCNC(=O)c2cc3ncccn3n2)n1] | N-(3-(4-(4-(3-amino-6-(2- hydroxyphenyl)pyridazin-4-yl)-1,4- diazepan-1-yl)-6- (dimethylamino)pyrimidin-2-yl)prop-2- yn-1-yl)pyrazolo[1,5-a]pyrimidine-2- carboxamide |
| P-140 | 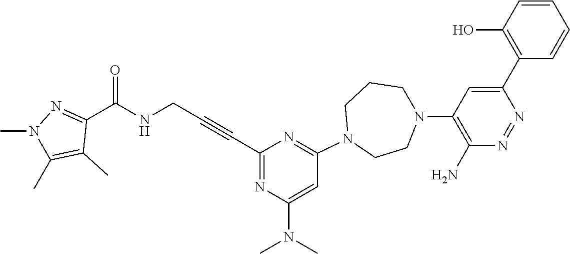 [SMILES: Cc1c(C(=O)NCC#Cc2nc(N(C)C)cc(N3CCCN(c4cc(-c5ccccc5O)nnc4N)CC3)n2)nn(C)c1C] | N-(3-(4-(4-(3-amino-6-(2- hydroxyphenyl)pyridazin-4-yl)-1,4- diazepan-1-yl)-6- (dimethylamino)pyrimidin-2-yl)prop-2- yn-1-yl)-1,4,5-trimethyl-1H-pyrazole-3- carboxamide |
| P-141 | 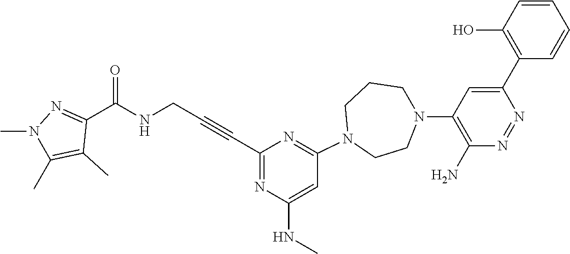 [SMILES: CNc1cc(N2CCCN(c3cc(-c4ccccc4O)nnc3N)CC2)nc(C#CCNC(=O)c2nn(C)c(C)c2C)n1] | N-(3-(4-(4-(3-amino-6-(2- hydroxyphenyl)pyridazin-4-yl)-1,4- diazepan-1-yl)-6- (methylamino)pyrimidin-2-yl)prop-2-yn- 1-yl)-1,4,5-trimethyl-1H-pyrazole-3- carboxamide |
| P-142 | 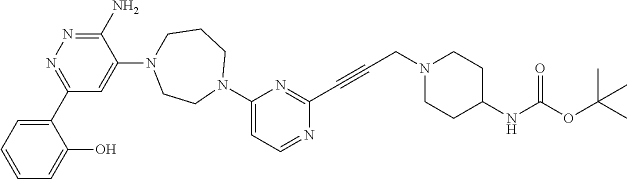 [SMILES: CC(C)(C)OC(=O)NC1CCN(CC#Cc2nccc(N3CCCN(c4cc(-c5ccccc5O)nnc4N)CC3)n2)CC1] | tert-butyl (1-(3-(4-(4-(3-amino-6-(2- hydroxyphenyl)pyridazin-4-yl)-1,4- diazepan-1-yl)pyrimidin-2-yl)prop-2-yn- 1-yl)piperidin-4-yl)carbamate |
| P-143 | 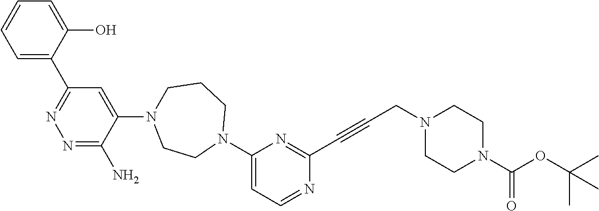 [SMILES: CC(C)(C)OC(=O)N1CCN(CC#Cc2nccc(N3CCCN(c4cc(-c5ccccc5O)nnc4N)CC3)n2)CC1] | tert-butyl 4-(3-(4-(4-(3-amino-6-(2- hydroxyphenyl)pyridazin-4-yl)-1,4- diazepan-1-yl)pyrimidin-2-yl)prop-2-yn- 1-yl)piperazine-1-carboxylate |
| P-144 | 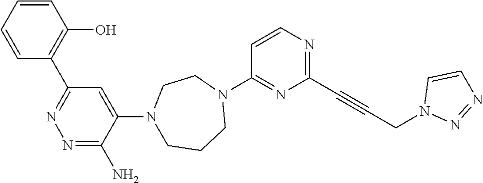 [SMILES: Nc1nnc(-c2ccccc2O)cc1N1CCCN(c2ccnc(C#CCn3ccnn3)n2)CC1] | 2-(5-(4-(2-(3-(1H-1,2,3-triazol-1-yl)prop- 1-yn-1-yl)pyrimidin-4-yl)-1,4-diazepan- 1-yl)-6-aminopyridazin-3-yl)phenol |
| P-145 | 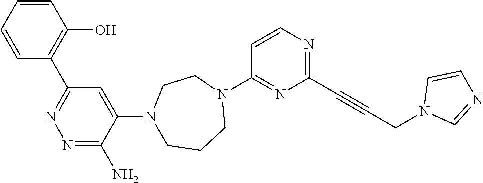 [SMILES: Nc1nnc(-c2ccccc2O)cc1N1CCCN(c2ccnc(C#CCn3ccnc3)n2)CC1] | 2-(5-(4-(2-(3-(1H-imidazol-1-yl)prop-1- yn-1-yl)pyrimidin-4-yl)-1,4-diazepan-1- yl)-6-aminopyridazin-3-yl)phenol |
| P-146 | 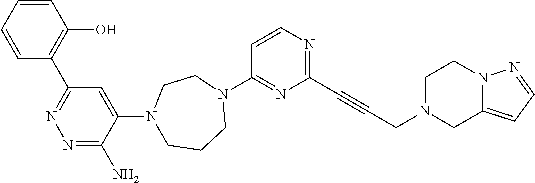 [SMILES: Nc1nnc(-c2ccccc2O)cc1N1CCCN(c2ccnc(C#CCN3CCn4nccc4C3)n2)CC1] | 2-(6-amino-5-(4-(2-(3-(6,7- dihydropyrazolo[1,5-a]pyrazin-5(4H)- yl)prop-1-yn-1-yl)pyrimidin-4-yl)-1,4- diazepan-1-yl)pyridazin-3-yl)phenol |
| P-147 | 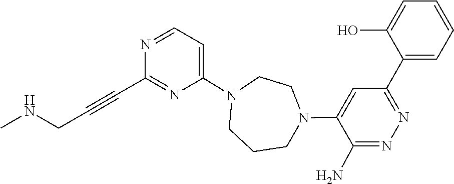 [SMILES: CNCC#Cc1nccc(N2CCCN(c3cc(-c4ccccc4O)nnc3N)CC2)n1] | 2-(6-amino-5-(4-(2-(3- (methylamino)prop-1-yn-1-yl)pyrimidin- 4-yl)-1,4-diazepan-1-yl)pyridazin-3- yl)phenol |
| P-148 | 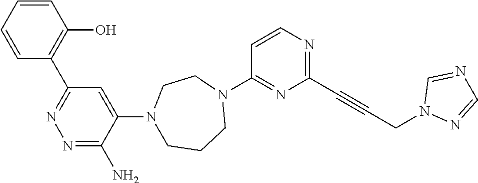 [SMILES: Nc1nnc(-c2ccccc2O)cc1N1CCCN(c2ccnc(C#CCn3cncn3)n2)CC1] | 2-(5-(4-(2-(3-(1H-1,2,4-triazol-1-yl)prop- 1-yn-1-yl)pyrimidin-4-yl)-1,4-diazepan- 1-yl)-6-aminopyridazin-3-yl)phenol |
| P-149 | 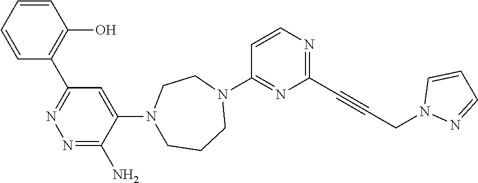 [SMILES: Nc1nnc(-c2ccccc2O)cc1N1CCCN(c2ccnc(C#CCn3cccn3)n2)CC1] | 2-(5-(4-(2-(3-(1H-pyrazol-1-yl)prop-1- yn-1-yl)pyrimidin-4-yl)-1,4-diazepan-1- yl)-6-aminopyridazin-3-yl)phenol |
| P-150 | 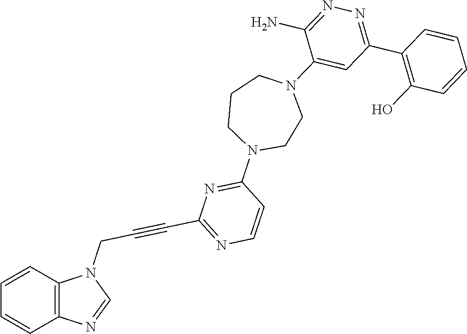 [SMILES: Nc1nnc(-c2ccccc2O)cc1N1CCCN(c2ccnc(C#CCn3cnc4ccccc43)n2)CC1] | 2-(5-(4-(2-(3-(1H-benzo[d]imidazol-1- yl)prop-1-yn-1-yl)pyrimidin-4-yl)-1,4- diazepan-1-yl)-6-aminopyridazin-3- yl)phenol |
| P-151 | 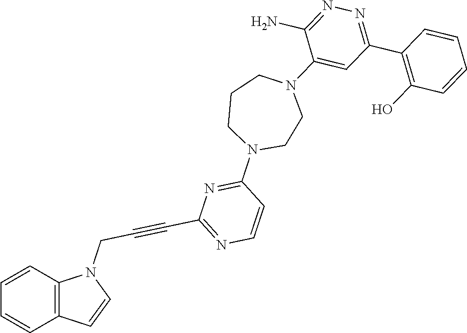 [SMILES: Nc1nnc(-c2ccccc2O)cc1N1CCCN(c2ccnc(C#CCn3ccc4ccccc43)n2)CC1] | 2-(5-(4-(2-(3-(1H-indol-1-yl)prop-1-yn-1- yl)pyrimidin-4-yl)-1,4-diazepan-1-yl)-6- aminopyridazin-3-yl)phenol |
| P-152 | 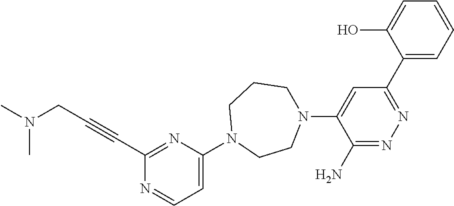 [SMILES: CN(C)CC#Cc1nccc(N2CCCN(c3cc(-c4ccccc4O)nnc3N)CC2)n1] | 2-(6-amino-5-(4-(2-(3- (dimethylamino)prop-1-yn-1- yl)pyrimidin-4-yl)-1,4-diazepan-1- yl)pyridazin-3-yl)phenol |

Also provided herein are the following compounds within the scope of Formula I, or their pharmaceutically acceptable salts, solvates, hydrates, or tautomers thereof.

| | |
|---|---|
| P-1 | N-[3-(4-{4-[3-amino-6-(2-hydroxyphenyl)pyridazin-4-yl]piperazin-1-yl}pyrimidin-2- |
| | yl)prop-2-yn-1-yl]-5-methyl-1,2-oxazole-3-carboxamide |
| P-2 | N-[3-(4-{4-[3-amino-6-(2-hydroxyphenyl)pyridazin-4-yl]pipcrazin-1-yl}pyrimidin-2- |
| | yl)prop-2-yn-1-yl]-5-ethyl-1-methyl-1H-pyrazole-3-carboxamide |
| P-3 | N-[3-(4-{4-[3-amino-6-(2-hydroxyphenyl)pyridazin-4-yl]piperazin-1-yl}pyrimidin-2- |
| | yl)prop-2-yn-1-yl]-5-methyl-1,2-oxazole-4-carboxamide |
| P-4 | N-[3-(4-{4-[3-amino-6-(2-hydroxyphenyl)pyridazin-4-yl]piperazin-1-yl}pyrimidin-2- |
| | yl)prop-2-yn-1-yl]pyridine-3-carboxamide |
| P-5 | N-[3-(4-{4-[3-amino-6-(2-hydroxyphenyl)pyridazin-4-yl]piperazin-1-yl}pyrimidin-2- |
| | yl)prop-2-yn-1-yl]pyridazine-3-carboxamide |
| P-6 | N-[3-(4-{4-[3-amino-6-(2-hydroxyphenyl)pyridazin-4-yl]piperazin-1-yl}pyrimidin-2- |
| | yl)prop-2-yn-1-yl]-5-cyclopropyl-1-methyl-1H-pyrazole-3-carboxamide |
| P-7 | N-[3-(4-{4-[3-amino-6-(2-hydroxyphenyl)pyridazin-4-yl]piperazin-1-yl}pyrimidin-2- |
| | yl)prop-2-yn-1-yl]-1-methyl-1H-pyrazole-3-carboxamide |
| P-8 | N-[3-(4-{4-[3-amino-6-(2-hydroxyphenyl)pyridazin-4-yl]piperazin-1-yl}pyrimidin-2- |
| | yl)prop-2-yn-1-yl]-1,4,5-trimethyl-1H-pyrazole-3-carboxamide |
| P-9 | N-[3-(4-{4-[3-amino-6-(2-hydroxyphenyl)pyridazin-4-yl]piperazin-1-yl}pyrimidin-2- |
| | yl)prop-2-yn-1-yl]-1-(2,2,2-trifluoroethyl)-1H-pyrazole-3-carboxamide |
| P-10 | N-[3-(4-{4-[3-amino-6-(2-hydroxyphenyl)pyridazin-4-yl]piperazin-1-yl}pyrimidin-2- |
| | yl)prop-2-yn-1-yl]-1-(difluoromethyl)-1H-pyrazole-3-carboxamide |
| P-11 | N-[3-(4-{4-[3-amino-6-(2-hydroxyphenyl)pyridazin-4-yl]piperazin-1-yl}pyrimidin-2- |
| | yl)prop-2-yn-1-yl]-1-cyclopropyl-1H-pyrazole-3-carboxamide |
| P-12 | N-[3-(4-{4-[3-amino-6-(2-hydroxyphenyl)pyridazin-4-yl]piperazin-1-yl}pyrimidin-2- |
| | yl)prop-2-yn-1-yl]-1,5-dimethyl-1H-pyrazole-3-carboxamide |
| P-14 | (R)-N-(3-(4-(4-(3-amino-6-(2-hydroxyphenyl)pyridazin-4-yl)-7-methyl-1,4-diazepan-1- |
| | yl)pyrimidin-2-yl)prop-2-yn-1-yl)pyrazolo[1,5-a]pyrimidine-2-carboxamide |
| P-16 | N-(3-(4-((3S,5S)-4-(3-amino-6-(2-hydroxyphenyl)pyridazin-4-yl)-3,5- |
| | dimethylpiperazin-1-yl)pyrimidin-2-yl)prop-2-yn-1-yl)pyrazolo[1,5-a]pyrimidine-2- |
| | carboxamide |
| P-17 | N-(3-(4-(6-(3-amino-6-(2-hydroxyphenyl)pyridazin-4-yl)-3,6-diazabicyclo[3.2.1]octan- |
| | 3-yl)pyrimidin-2-yl)prop-2-yn-1-yl)pyrazolo[1,5-a]pyrimidine-2-carboxamide |
| P-18 | N-(3-(4-(6-(3-amino-6-(2-hydroxyphenyl)pyridazin-4-yl)-2,6-diazabicyclo[3.2.0]heptan- |
| | 2-yl)pyrimidin-2-yl)prop-2-yn-1-yl)pyrazolo[1,5-a]pyrimidine-2-carboxamide |
| P-19 | N-(3-(4-(4-(3-amino-6-(2-hydroxyphenyl)pyridazin-4-yl)-5-methyl-1,4-diazepan-1- |
| | yl)pyrimidin-2-yl)prop-2-yn-1-yl)pyrazolo[1,5-a]pyrimidine-2-carboxamide |
| P-20 | N-(3-(4-(4-(3-amino-6-(2-hydroxyphenyl)pyridazin-4-yl)-2-methyl-1,4-diazepan-1- |
| | yl)pyrimidin-2-yl)prop-2-yn-1-yl)pyrazolo[1,5-a]pyrimidine-2-carboxamide |
| P-22 | N-(4-(4-(8-(3-amino-6-(2-hydroxyphenyl)pyridazin-4-yl)-3,8-diazabicyclo[3.2.1]octan- |
| | 3-yl)pyrimidin-2-yl)but-3-yn-1-yl)pyrazolo[1,5-a]pyrimidine-2-carboxamide |
| P-23 | N-(4-(4-(4-(3-amino-6-(2-hydroxyphenyl)pyridazin-4-yl)-1,4-diazepan-1-yl)pyrimidin- |
| | 2-yl)but-3-yn-1-yl)pyrazolo[1,5-a]pyrimidine-2-carboxamide |
| P-24 | N-(3-(4-(4-(3-amino-6-(2-hydroxyphenyl)pyridazin-4-yl)-1,4-diazepan-1-yl)pyrimidin- |
| | 2-yl)prop-2-yn-1-yl)-N-methylpyrazolo[1,5-a]pyrimidine-2-carboxamide |
| P-25 | N-(3-(4-((1R,4R)-5-(3-amino-6-(2-hydroxyphenyl)pyridazin-4-yl)-2,5- |
| | diazabicyclo[2.2.1]heptan-2-yl)pyrimidin-2-yl)prop-2-yn-1-yl)pyrazolo[1,5- |
| | a]pyrimidine-2-carboxamide |
| P-27 | N-(3-(4-(4-(3-amino-6-(2-hydroxyphenyl)pyridazin-4-yl)-6-methyl-1,4-diazepan-1- |
| | yl)pyrimidin-2-yl)prop-2-yn-1-yl)pyrazolo[1,5-a]pyrimidine-2-carboxamide |
| P-28 | N-(3-(4-(5-(3-amino-6-(2-hydroxyphenyl)pyridazin-4-yl)-2,5-diazabicyclo[4.1.0]heptan- |
| | 2-yl)pyrimidin-2-yl)prop-2-yn-1-yl)pyrazolo[1,5-a]pyrimidine-2-carboxamide |
| P-29 | N-(3-(4-(4-(3-amino-6-(2-hydroxyphcnyl)pyridazin-4-yl)-3-mcthyl-1,4-diazcpan-1- |
| | yl)pyrimidin-2-yl)prop-2-yn-1-yl)pyrazolo[1,5-a]pyrimidine-2-carboxamide |
| P-30 | N-(3-(4-((1S,4S)-5-(3-amino-6-(2-hydroxyphenyl)pyridazin-4-yl)-2,5- |
| | diazabicyclo[2.2.1]heptan-2-yl)pyrimidin-2-yl)prop-2-yn-1-yl)pyrazolo[1,5- |
| | a]pyrimidine-2-carboxamide |
| P-31 | N-(3-(4-(2-(3-amino-6-(2-hydroxyphenyl)pyridazin-4-yl)-2,7-diazaspiro[3.5]nonan-7- |
| | yl)pyrimidin-2-yl)prop-2-yn-1-yl)pyrazolo[1,5-a]pyrimidine-2-carboxamide |
| P-32 | N-(3-(4-(8-(3-amino-6-(2-hydroxyphenyl)pyridazin-4-yl)-2,8-diazaspiro[4.5]decan-2- |
| | yl)pyrimidin-2-yl)prop-2-yn-1-yl)pyrazolo[1,5-a]pyrimidine-2-carboxamide |
| P-33 | N-(3-(4-(2-(3-amino-6-(2-hydroxyphenyl)pyridazin-4-yl)-2,9-diazaspiro[5.5]undecan-9- |
| | yl)pyrimidin-2-yl)prop-2-yn-1-yl)pyrazolo[1,5-a]pyrimidine-2-carboxamide |
| P-34 | N-(3-(4-(2-(3-amino-6-(2-hydroxyphenyl)pyridazin-4-yl)-2,8-diazaspiro[4.5]decan-8- |
| | yl)pyrimidin-2-yl)prop-2-yn-1-yl)pyrazolo[1,5-a]pyrimidine-2-carboxamide |
| P-35 | N-(3-(4-(9-(3-amino-6-(2-hydroxyphenyl)pyridazin-4-yl)-2,9-diazaspiro[5.5]undecan-2- |
| | yl)pyrimidin-2-yl)prop-2-yn-1-yl)pyrazolo[1,5-a]pyrimidine-2-carboxamide |
| P-36 | N-(3-(4-(5-(3-amino-6-(2-hydroxyphenyl)pyridazin-4-yl)hexahydropyrrolo[3,4-b]pyrrol- |
| | 1(2H)-yl)pyrimidin-2-yl)prop-2-yn-1-yl)pyrazolo[1,5-a]pyrimidine-2-carboxamide |
| P-37 | N-(3-(4-(6-(3-amino-6-(2-hydroxyphenyl)pyridazin-4-yl)octahydro-1H-pyrrolo[2,3- |
| | c]pyridin-1-yl)pyrimidin-2-yl)prop-2-yn-1-yl)pyrazolo[1,5-a]pyrimidine-2-carboxamide |
| P-38 | N-(3-(4-(1-(3-amino-6-(2-hydroxyphenyl)pyridazin-4-yl)octahydro-4H-pyrrolo\|3,2- |
| | b]pyridin-4-yl)pyrimidin-2-yl)prop-2-yn-1-yl)pyrazolo[1,5-a]pyrimidine-2-carboxamide |
| P-41 | N-(3-(4-(5-(3-amino-6-(2-hydroxyphenyl)pyridazin-4-yl)hexahydropyrrolo[3,4-c]pyrrol- |
| | 2(1H)-yl)pyrimidin-2-yl)prop-2-yn-1-yl)pyrazolo[1,5-a]pyrimidine-2-carboxamide |
| P-42 | N-(3-(4-(9-(3-amino-6-(2-hydroxyphenyl)pyridazin-4-yl)-3,9-diazaspiro[5.5]undecan-3- |
| | yl)pyrimidin-2-yl)prop-2-yn-1-yl)pyrazolo[1,5-a]pyrimidine-2-carboxamide |
| P-43 | N-(3-(4-(3-(3-amino-6-(2-hydroxyphenyl)pyridazin-4-yl)-3,6-diazabicyclo[3.2.0]heptan- |
| | 6-yl)pyrimidin-2-yl)prop-2-yn-1-yl)pyrazolo[1,5-a]pyrimidine-2-carboxamide |
| P-44 | N-(3-(4-(4-(3-amino-6-(2-hydroxyphenyl)pyridazin-4-yl)-6-fluoro-1,4-diazepan-1- |
| | yl)pyrimidin-2-yl)prop-2-yn-1-yl)pyrazolo[1,5-a]pyrimidine-2-carboxamide |
| P-45 | N-(3-(4-(6-(3-amino-6-(2-hydroxyphenyl)pyridazin-4-yl)-3,6-diazabicyclo[3.2.0]heptan- |
| | 3-yl)pyrimidin-2-yl)prop-2-yn-1-yl)pyrazolo[1,5-a]pyrimidine-2-carboxamide |
| P-46 | N-(3-(4-(4-(3-amino-6-(2-hydroxyphenyl)pyridazin-4-yl)-7-methyl-1,4-diazepan-1- |
| | yl)pyrimidin-2-yl)prop-2-yn-1-yl)pyrazolo[1,5-a]pyrimidine-2-carboxamide |
| P-47 | N-(3-(4-((1R,5R)-2-(3-amino-6-(2-hydroxyphenyl)pyridazin-4-yl)-2,6- |
| | diazabicyclo[3.2.0]hcptan-6-yl)pyrimidin-2-yl)prop-2-yn-1-yl)pyrazolo[1,5- |
| | a]pyrimidine-2-carboxamide |
| P-48 | (S)-N-(3-(4-(4-(3-amino-6-(2-hydroxyphenyl)pyridazin-4-yl)-3-methylpiperazin-1- |
| | yl)pyrimidin-2-yl)prop-2-yn-1-yl)pyrazolo[1,5-a]pyrimidine-2-carboxamide |
| P-49 | (R)-N-(3-(4-(4-(3-amino-6-(2-hydroxyphenyl)pyridazin-4-yl)-3-methylpiperazin-1- |
| | yl)pyrimidin-2-yl)prop-2-yn-1-yl)pyrazolo[1,5-a]pyrimidine-2-carboxamide |
| P-50 | (R)-N-(3-(4-(4-(3-amino-6-(2-hydroxyphenyl)pyridazin-4-yl)-2-methylpiperazin-1- |
| | yl)pyrimidin-2-yl)prop-2-yn-1-yl)pyrazolo[1,5-a]pyrimidine-2-carboxamide |
| P-51 | N-(3-(4-(4-(3-amino-6-(2-hydroxyphenyl)pyridazin-4-yl)-3-ethylpiperazin-1- |
| | yl)pyrimidin-2-yl)prop-2-yn-1-yl)pyrazolo[1,5-a]pyrimidine-2-carboxamide |
| P-52 | N-(3-(4-(4-(3-amino-6-(2-hydroxyphenyl)pyridazin-4-yl)piperazin-1-yl)pyrimidin-2- |
| | yl)prop-2-yn-1-yl)-6-fluorobenzofuran-2-carboxamide |
| P-53 | N-(3-(4-(4-(3-amino-6-(2-hydroxyphenyl)pyridazin-4-yl)piperazin-1-yl)pyrimidin-2- |
| | yl)prop-2-yn-1-yl)-6-fluorobenzofuran-2-carboxamide |
| P-54 | N-(3-(4-(4-(3-amino-6-(2-hydroxyphenyl)pyridazin-4-yl)piperazin-1-yl)pyrimidin-2- |
| | yl)prop-2-yn-1-yl)benzo[d]isoxazole-3-carboxamide |
| P-55 | N-(3-(4-(4-(3-amino-6-(2-hydroxyphenyl)pyridazin-4-yl)piperazin-1-yl)pyrimidin-2- |
| | yl)prop-2-yn-1-yl)benzofuran-3-carboxamide |
| P-56 | N-(3-(4-(4-(3-amino-6-(2-hydroxyphenyl)pyridazin-4-yl)piperazin-1-yl)pyrimidin-2- |
| | yl)prop-2-yn-1-yl)isonicotinamide |
| P-60 | N-(3-(4-(4-(3-amino-6-(2-hydroxyphenyl)pyridazin-4-yl)piperazin-1-yl)pyrimidin-2- |
| | yl)prop-2-yn-1-yl)imidazo[1,2-a]pyrazine-2-carboxamide |
| P-62 | N-(3-(4-(4-(3-amino-6-(2-hydroxyphenyl)pyridazin-4-yl)piperazin-1-yl)pyrimidin-2- |
| | yl)prop-2-yn-1-yl)-4-methylthiazole-5-carboxamide |
| P-63 | N-(3-(4-(4-(3-amino-6-(2-hydroxyphenyl)pyridazin-4-yl)piperazin-1-yl)pyrimidin-2- |
| | yl)prop-2-yn-1-yl)benzofuran-7-carboxamide |
| P-64 | N-(3-(4-(4-(3-amino-6-(2-hydroxyphenyl)pyridazin-4-yl)piperazin-1-yl)pyrimidin-2- |
| | yl)prop-2-yn-1-yl)quinoline-2-carboxamide |
| P-65 | N-(3-(4-(4-(3-amino-6-(2-hydroxyphenyl)pyridazin-4-yl)piperazin-1-yl)pyrimidin-2- |
| | yl)prop-2-yn-1-yl)pyrazolo[1,5-a]pyridine-3-carboxamide |
| P-66 | N-(3-(4-(4-(3-amino-6-(2-hydroxyphenyl)pyridazin-4-yl)piperazin-1-yl)pyrimidin-2- |
| | yl)prop-2-yn-1-yl)imidazo[1,2-a]pyridine-3-carboxamide |
| P-67 | N-(3-(4-(4-(3-amino-6-(2-hydroxyphenyl)pyridazin-4-yl)piperazin-1-yl)pyrimidin-2- |
| | yl)prop-2-yn-1-yl)-1-methyl-1H-imidazole-4-carboxamide |
| P-68 | N-(3-(4-(4-(3-amino-6-(2-hydroxyphenyl)pyridazin-4-yl)pipcrazin-1-yl)pyrimidin-2- |
| | yl)prop-2-yn-1-yl)acetamide |
| P-70 | N-(3-(4-(4-(3-amino-6-(2-hydroxyphenyl)pyridazin-4-yl)piperazin-1-yl)pyrimidin-2- |
| | yl)prop-2-yn-1-yl)-5,7-dimethylpyrazolo[1,5-a]pyrimidine-3-carboxamide |
| P-71 | N-(3-(4-(4-(3-amino-6-(2-hydroxyphenyl)pyridazin-4-yl)piperazin-1-yl)pyrimidin-2- |
| | yl)prop-2-yn-1-yl)pyrazolo[1,5-a]pyrimidine-3-carboxamide |
| P-72 | N-(3-(4-(4-(3-amino-6-(2-hydroxyphenyl)pyridazin-4-yl)piperazin-1-yl)pyrimidin-2- |
| | yl)prop-2-yn-1-yl)quinoxaline-2-carboxamide |
| P-73 | N-(3-(4-(4-(3-amino-6-(2-hydroxyphenyl)pyridazin-4-yl)piperazin-1-yl)pyrimidin-2- |
| | yl)prop-2-yn-1-yl)-1H-indole-2-carboxamide |
| P-74 | N-(3-(4-(4-(3-amino-6-(2-hydroxyphenyl)pyridazin-4-yl)piperazin-1-yl)pyrimidin-2- |
| | yl)prop-2-yn-1-yl)-3-methylbenzofuran-2-carboxamide |
| P-75 | N-(3-(4-(4-(3-amino-6-(2-hydroxyphenyl)pyridazin-4-yl)piperazin-1-yl)pyrimidin-2- |
| | yl)prop-2-yn-1-yl)benzofuran-2-carboxamide |
| P-76 | N-(3-(4-(4-(3-amino-6-(2-hydroxyphenyl)pyridazin-4-yl)-1,4-diazepan-1-yl)pyrimidin- |
| | 2-yl)prop-2-yn-1-yl)pyrazolo[1,5-a]pyrimidine-2-carboxamide |
| P-77 | N-(3-(4-(4-(3-amino-6-(2-hydroxyphenyl)pyridazin-4-yl)hexahydropyrrolo[3,2-b]pyrrol- |
| | 1(2H)-yl)pyrimidin-2-yl)prop-2-yn-1-yl)pyrazolo[1,5-a]pyrimidine-2-carboxamide |
| P-78 | N-(3-(4-(8-(3-amino-6-(2-hydroxyphenyl)pyridazin-4-yl)-3,8-diazabicyclo[3.2.1]octan- |
| | 3-yl)pyrimidin-2-yl)prop-2-yn-1-yl)pyrazolo\|1,5-a\|pyrimidine-2-carboxamide |
| P-79 | N-(3-(4-(4-(3-amino-6-(2-hydroxyphenyl)pyridazin-4-yl)octahydro-1H-pyrrolo[3,2- |
| | b]pyridin-1-yl)pyrimidin-2-yl)prop-2-yn-1-yl)pyrazolo[1,5-a]pyrimidine-2-carboxamide |
| P-80 | N-(3-(4-(6-(3-amino-6-(2-hydroxyphenyl)pyridazin-4-yl)-3,6-diazabicyclo[3.1.1]heptan- |
| | 3-yl)pyrimidin-2-yl)prop-2-yn-1-yl)pyrazolo[1,5-a]pyrimidine-2-carboxamide |
| P-81 | (R)-N-(3-(4-(4-(3-amino-6-(2-hydroxyphenyl)pyridazin-4-yl)-2-methylpiperazin-1- |
| | yl)pyrimidin-2-yl)prop-2-yn-1-yl)pyrazolo[1,5-a]pyrimidine-2-carboxamide |
| P-82 | N-(3-(4-((3R,5R)-4-(3-amino-6-(2-hydroxyphenyl)pyridazin-4-yl)-3,5- |
| | dimethylpiperazin-1-yl)pyrimidin-2-yl)prop-2-yn-1-yl)pyrazolo[1,5-a]pyrimidine-2- |
| | carboxamide |
| P-83 | N-(3-(4-(4-(3-amino-6-(2-hydroxyphenyl)pyridazin-4-yl)-3,3-dimethylpiperazin-1- |
| | yl)pyrimidin-2-yl)prop-2-yn-1-yl)pyrazolo[1,5-a]pyrimidine-2-carboxamide |
| P-92 | N-(5-(4-(4-(3-amino-6-(2-hydroxyphenyl)pyridazin-4-yl)piperazin-1-yl)pyrimidin-2- |
| | yl)pent-4-yn-1-yl)pyrazolo[1,5-a]pyrimidine-2-carboxamide |
| P-93 | N-(3-(4-(4-(3-amino-6-(2-hydroxyphenyl)pyridazin-4-yl)piperazin-1-yl)pyrimidin-2- |
| | yl)prop-2-yn-1-yl)pyrazolo[1,5-a]pyrimidinc-2-carboxamidc |
| P-94 | N-(4-(4-(4-(3-amino-6-(2-hydroxyphenyl)pyridazin-4-yl)piperazin-1-yl)pyrimidin-2- |
| | yl)but-3-yn-1-yl)pyrazolo[1,5-a]pyrimidine-2-carboxamide |
| P-95 | N-(4-(4-(4-(3-amino-6-(2-hydroxyphenyl)pyridazin-4-yl)piperazin-1-yl)pyrimidin-2- |
| | yl)but-3-yn-2-yl)pyrazolo[1,5-a]pyrimidine-2-carboxamide |
| P-96 | N-(4-(4-(3-(3-amino-6-(2-hydroxyphenyl)pyridazin-4-yl)-3,8-diazabicyclo[3.2.1]octan- |
| | 8-yl)pyrimidin-2-yl)but-3-yn-1-yl)pyrazolo[1,5-a]pyrimidine-2-carboxamide |
| P-97 | N-(4-(4-(3-(3-amino-6-(2-hydroxyphenyl)pyridazin-4-yl)-3,8-diazabicyclo[3.2.1]octan- |
| | 8-yl)pyrimidin-2-yl)but-3-yn-2-yl)pyrazolo[1,5-a]pyrimidine-2-carboxamide |
| P-98 | N-(1-((4-(3-(3-amino-6-(2-hydroxyphenyl)pyridazin-4-yl)-3,8-diazabicyclo[3.2.1]octan- |
| | 8-yl)pyrimidin-2-yl)ethynyl)cyclopropyl)pyrazolo[1,5-a]pyrimidine-2-carboxamide |
| P-99 | N-(5-(4-(3-(3-amino-6-(2-hydroxyphenyl)pyridazin-4-yl)-3,8-diazabicyclo[3.2.1]octan- |
| | 8-yl)pyrimidin-2-yl)pent-4-yn-1-yl)pyrazolo[1,5-a]pyrimidine-2-carboxamide |
| P-101 | (S)-N-(3-(4-(4-(3-amino-6-(2-hydroxyphenyl)pyridazin-4-yl)-7-methyl-1,4-diazepan-1- |
| | yl)pyrimidin-2-yl)prop-2-yn-1-yl)quinoxaline-2-carboxamide |
| P-102 | (S)-N-(3-(4-(4-(3-amino-6-(2-hydroxyphenyl)pyridazin-4-yl)-7-methyl-1,4-diazepan-1- |
| | yl)pyrimidin-2-yl)prop-2-yn-1-yl)pyrazolo[1,5-a]pyrimidine-2-carboxamide |
| P-104 | 2-(6-amino-5-(4-(2-(3-(3,5-diisopropyl-1H-pyrazol-1-yl)prop-1-yn-1-yl)pyrimidin-4-yl)- |
| | 1,4-diazepan-1-yl)pyridazin-3-yl)phenol |
| P-106 | 2-(6-amino-5-(8-(2-(3-(oxazol-2-ylamino)prop-1-yn-1-yl)pyrimidin-4-yl)-3,8- |
| | diazabicyclo[3.2.1]octan-3-yl)pyridazin-3-yl)phenol |
| P-107 | 2-(6-amino-5-(4-(2-(3-(benzo[d]oxazol-2-yl(methyl)amino)prop-1-yn-1-yl)pyrimidin-4- |
| | yl)-1,4-diazepan-1-yl)pyridazin-3-yl)phenol |
| P-108 | 2-(6-amino-5-(4-(2-(3-((1-methyl-1H-pyrazol-3-yl)oxy)prop-1-yn-1-yl)pyrimidin-4-yl)- |
| | 1,4-diazepan-1-yl)pyridazin-3-yl)phenol |
| P-110 | (E)-N-(3-(4-(4-(3-amino-6-(2-hydroxyphenyl)pyridazin-4-yl)-1,4-diazepan-1- |
| | yl)pyrimidin-2-yl)allyl)quinoxaline-2-carboxamide |
| P-111 | (E)-N-(3-(4-(4-(3-amino-6-(2-hydroxyphenyl)pyridazin-4-yl)-1,4-diazepan-1- |
| | yl)pyrimidin-2-yl)allyl)-1,4,5-trimethyl-1H-pyrazole-3-carboxamide |
| P-112 | (E)-N-(3-(4-(4-(3-amino-6-(2-hydroxyphenyl)pyridazin-4-yl)-1,4-diazepan-1- |
| | yl)pyrimidin-2-yl)allyl)pyrazolo[1,5-a]pyrimidine-2-carboxamide |
| P-113 | (E)-2-(6-amino-5-(4-(2-(3-aminoprop-1-en-1-yl)pyrimidin-4-yl)-1,4-diazepan-1- |
| | yl)pyridazin-3-yl)phenol |
| P-114 | tert-butyl (E)-(3-(4-(4-(3-amino-6-(2-hydroxyphcnyl)pyridazin-4-yl)-1,4-diazcpan-1- |
| | yl)pyrimidin-2-yl)allyl)carbamate |
| P-116 | 2-(5-(4-(2-(3-(1H-pyrazolo[3,4-c]pyridin-1-yl)prop-1-yn-1-yl)pyrimidin-4-yl)-1,4- |
| | diazepan-1-yl)-6-aminopyridazin-3-yl)phenol |
| P-117 | N-(3-(4-(4-(3-amino-6-(2-hydroxyphenyl)pyridazin-4-yl)piperazin-1-yl)pyrimidin-2- |
| | yl)prop-2-yn-1-yl)-4-fluoropyrazolo[1,5-a]pyridine-3-carboxamide |
| P-118 | N-(3-(4-(4-(3-amino-6-(2-hydroxyphenyl)pyridazin-4-yl)piperazin-1-yl)pyrimidin-2- |
| | yl)prop-2-yn-1-yl)benzo[d]thiazole-2-carboxamide |
| P-119 | N-(3-(4-(3-(3-amino-6-(2-hydroxyphenyl)pyridazin-4-yl)-3,8-diazabicyclo[3.2.1]octan- |
| | 8-yl)pyrimidin-2-yl)prop-2-yn-1-yl)pyrazolo[1,5-a]pyrimidine-2-carboxamide |
| P-121 | tert-butyl (3-(4-(4-(3-amino-6-(2-hydroxyphenyl)pyridazin-4-yl)-1,4-diazepan-1- |
| | yl)pyrimidin-2-yl)prop-2-yn-1-yl)carbamate |
| P-122 | N-(3-(4-(4-(3-amino-6-(2-hydroxyphenyl)pyridazin-4-yl)-1,4-diazepan-1-yl)pyrimidin- |
| | 2-yl)prop-2-yn-1-yl)acetamide |
| P-124 | N-(3-(4-(4-(3-amino-6-(2-hydroxyphenyl)pyridazin-4-yl)-1,4-diazepan-1-yl)-6- |
| | ethoxypyrimidin-2-yl)prop-2-yn-1-yl)pyrazolo[1,5-a]pyrimidine-2-carboxamide |
| P-126 | N-(3-(4-(4-(3-amino-6-(2-hydroxyphenyl)pyridazin-4-yl)-1,4-diazepan-1-yl)-6- |
| | (methoxymethyl)pyrimidin-2-yl)prop-2-yn-1-yl)-1,4,5-trimethyl-1H-pyrazole-3- |
| | carboxamide |
| P-127 | N-(3-4-(4-(3-amino-6-(2-hydroxyphenyl)pyridazin-4-yl)-1,4-diazepan-1-yl)-6- |
| | (methoxymethyl)pyrimidin-2-yl)prop-2-yn-1-yl)pyrazolo[1,5-a]pyrimidine-2- |
| | carboxamide |
| P-128 | 2-(6-amino-5-(4-(2-(3-(oxazol-2-yloxy)prop-1-yn-1-yl)pyrimidin-4-yl)-1,4-diazepan-1- |
| | yl)pyridazin-3-yl)phenol |
| P-131 | 2-(6-amino-5-(4-(2-(3-((1-methyl-1H-pyrazol-5-yl)oxy)prop-1-yn-1-yl)pyrimidin-4-yl)- |
| | 1,4-diazepan-1-yl)pyridazin-3-yl)phenol |
| P-132 | 2-(5-(8-(2-(3-(1H-benzo[d][1,2,3]triazol-1-yl)prop-1-yn-1-yl)pyrimidin-4-yl)-3,8- |
| | diazabicyclo[3.2.1]octan-3-yl)-6-aminopyridazin-3-yl)phenol |
| P-133 | 2-(5-(8-(2-(3-(1H-pyrazol-1-yl)prop-1-yn-1-yl)pyrimidin-4-yl)-3,8- |
| | diazabicyclo[3.2.1]octan-3-yl)-6-aminopyridazin-3-yl)phenol |
| P-138 | 2-(6-amino-5-(4-(2-(3-(3-(pyridin-2-yl)-1H-pyrazol-1-yl)prop-1-yn-1-yl)pyrimidin-4-yl)- |
| | 1,4-diazepan-1-yl)pyridazin-3-yl)phenol |
| P-139 | N-(3-(4-(4-(3-amino-6-(2-hydroxyphenyl)pyridazin-4-yl)-1,4-diazepan-1-yl)-6- |
| | (dimethylamino)pyrimidin-2-yl)prop-2-yn-1-yl)pyrazolo[1,5-a]pyrimidine-2- |
| | carboxamide |
| P-140 | N-(3-(4-(4-(3-amino-6-(2-hydroxyphenyl)pyridazin-4-yl)-1,4-diazepan-1-yl)-6- |
| | (dimethylamino)pyrimidin-2-yl)prop-2-yn-1-yl)-1,4,5-trimethyl-1H-pyrazole-3- |
| | carboxamide |
| P-141 | N-(3-(4-(4-(3-amino-6-(2-hydroxyphenyl)pyridazin-4-yl)-1,4-diazepan-1-yl)-6- |
| | (methylamino)pyrimidin-2-yl)prop-2-yn-1-yl)-1,4,5-trimethyl-1H-pyrazole-3- |
| | carboxamide |
| P-142 | tert-butyl (1-(3-(4-(4-(3-amino-6-(2-hydroxyphenyl)pyridazin-4-yl)-1,4-diazepan-1- |
| | yl)pyrimidin-2-yl)prop-2-yn-1-yl)piperidin-4-yl)carbamate |
| P-143 | tert-butyl 4-(3-(4-(4-(3-amino-6-(2-hydroxyphenyl)pyridazin-4-yl)-1,4-diazepan-1- |
| | yl)pyrimidin-2-yl)prop-2-yn-1-yl)piperazine-1-carboxylate |
| P-144 | 2-(5-(4-(2-(3-(1H-1,2,3-triazol-1-yl)prop-1-yn-1-yl)pyrimidin-4-yl)-1,4-diazepan-1-yl)- |
| | 6-aminopyridazin-3-yl)phenol |
| P-145 | 2-(5-(4-(2-(3-(1H-imidazol-1-yl)prop-1-yn-1-yl)pyrimidin-4-yl)-1,4-diazepan-1-yl)-6- |
| | aminopyridazin-3-yl)phenol |
| P-148 | 2-(5-(4-(2-(3-(1H-1,2,4-triazol-1-yl)prop-1-yn-1-yl)pyrimidin-4-yl)-1,4-diazepan-1-yl)- |
| | 6-aminopyridazin-3-yl)phenol |
| P-149 | 2-(5-(4-(2-(3-(1H-pyrazol-1-yl)prop-1-yn-1-yl)pyrimidin-4-yl)-1,4-diazepan-1-yl)-6- |
| | aminopyridazin-3-yl)phenol |
| P-150 | 2-(5-(4-(2-(3-(1H-benzo[d]imidazol-1-yl)prop-1-yn-1-yl)pyrimidin-4-yl)-1,4-diazepan- |
| | 1-yl)-6-aminopyridazin-3-yl)phenol |
| P-151 | 2-(5-(4-(2-(3-(1H-indol-1-yl)prop-1-yn-1-yl)pyrimidin-4-yl)-1,4-diazepan-1-yl)-6- |
| | aminopyridazin-3-yl)phenol |

Representative and non-limiting examples of compounds within the scope of Formula I or Formula I′ above are set forth in Table 1A, where each of which include their pharmaceutically acceptable salts, solvates, hydrates, or tautomers thereof.

TABLE 1A

| Compound | | |
|---|---|---|
| # | Structure | IUPAC Name |
| P-153 | 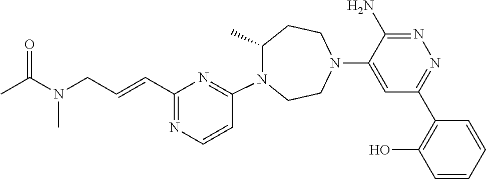 [SMILES: CC(=O)N(C)C/C=C/c1nccc(N2CCN(c3cc(-c4ccccc4O)nnc3N)CC[C@H]2C)n1] | N-[3-(4-{(R)-4-[3-amino-6-(o- hydroxyphenyl)-4-pyridazinyl]-7- methyl-1,4-diazepan-1-yl}-2- pyrimidinyl)-2-propenyl]-N- methylacetamide |
| P-154 | 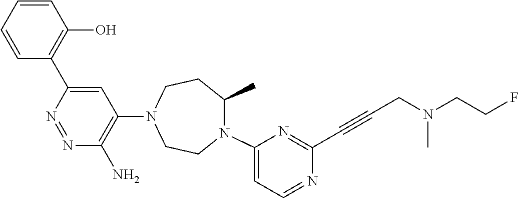 [SMILES: C[C@@H]1CCN(c2cc(-c3ccccc3O)nnc2N)CCN1c1ccnc(C#CCN(C)CCF)n1] | o-{5-[(R)-4-(2-{3-[(2-fluoroethyl)-N- methylamino]-1-propynyl}-4- pyrimidinyl)-5-methyl-1,4-diazepan- 1-yl]-6-amino-3-pyridazinyl}phenol |
| P-155 | 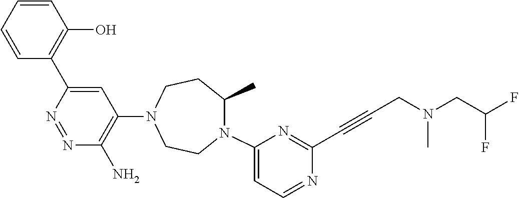 [SMILES: C[C@@H]1CCN(c2cc(-c3ccccc3O)nnc2N)CCN1c1ccnc(C#CCN(C)CC(F)F)n1] | o-{5-[(R)-4-(2-{3-[(2,2- difluoroethyl)-N-methylamino]-1- propynyl}-4-pyrimidinyl)-5-methyl- 1,4-diazepan-1-yl]-6-amino-3- pyridazinyl}phenol |
| P-156 | 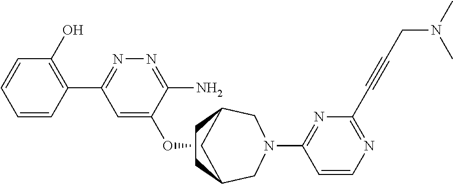 [SMILES: CN(C)CC#Cc1nccc(N2C[C@H]3CC[C@@H](C2)[C@H]3Oc2cc(-c3ccccc3O)nnc2N)n1] | o-{5-[(1R,5S,8r)-3-{2-[3- (dimethylamino)-1-propynyl]-4- pyrimidinyl}-3-azabicyclo[3.2.1]oct- 8-yloxy]-6-amino-3- pyridazinyl}phenol |
| P-157 | 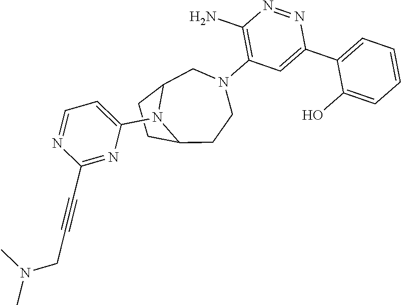 [SMILES: CN(C)CC#Cc1nccc(N2C3CCC2CN(c2cc(-c4ccccc4O)nnc2N)CC3)n1] | o-[6-amino-5-(9-{2-[3- (dimethylamino)-1-propynyl]-4- pyrimidinyl}-3,9- diazabicyclo[4.2.1]non-3-yl)-3- pyridazinyl]phenol |
| P-158 | 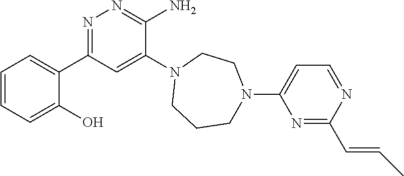 [SMILES: C/C=C/c1nccc(N2CCCN(c3cc(-c4ccccc4O)nnc3N)CC2)n1] | o-(6-amino-5-{4-[2-(1-propenyl)-4- pyrimidinyl]-1,4-diazepan-1-yl}-3- pyridazinyl)phenol |
| P-159 | 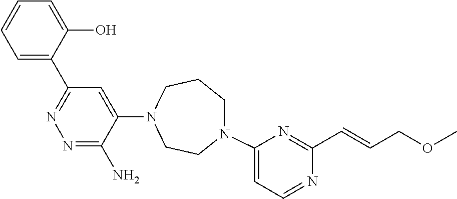 [SMILES: COC/C=C/c1nccc(N2CCCN(c3cc(-c4ccccc4O)nnc3N)CC2)n1] | o-(6-amino-5-{4-[2-(3-methoxy-1- propenyl)-4-pyrimidinyl]-1,4- diazepan-1-yl}-3-pyridazinyl)phenol |
| P-160 | 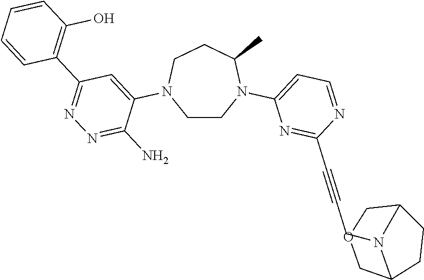 [SMILES: C[C@@H]1CCN(c2cc(-c3ccccc3O)nnc2N)CCN1c1ccnc(C#CCN2C3CCC2COC3)n1] | o-{5-[(R)-5-methyl-4-(2-{3-(3-oxa-8- azabicyclo[3.2.1]oct-8-yl)-1- propynyl}-4-pyrimidinyl)-1,4- diazepan-1-yl]-6-amino-3- pyridazinyl}phenol |
| P-161 | 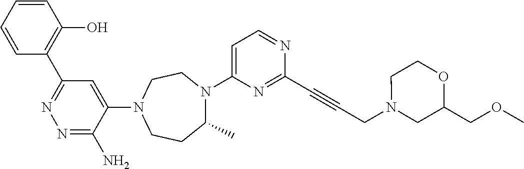 [SMILES: COCC1CN(CC#Cc2nccc(N3CCN(c4cc(-c5ccccc5O)nnc4N)CC[C@H]3C)n2)CCO1] | o-{5-[(R)-4-(2-{3-[2- (methoxymethyl)-4-morpholinyl]-1- propynyl}-4-pyrimidinyl)-5-methyl- 1,4-diazepan-1-yl]-6-amino-3- pyridazinyl}phenol |
| P-163 | 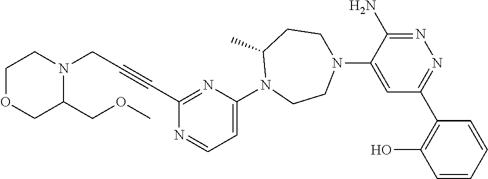 [SMILES: COCC1COCCN1CC#Cc1nccc(N2CCN(c3cc(-c4ccccc4O)nnc3N)CC[C@H]2C)n1] | o-{5-[(R)-4-(2-{3-[(2,2- difluoroethyl)-N-methylamino]-1- propenyl}-4-pyrimidinyl)-5-methyl- 1,4-diazepan-1-yl]-6-amino-3- pyridazinyl}phenol |
| P-162 | 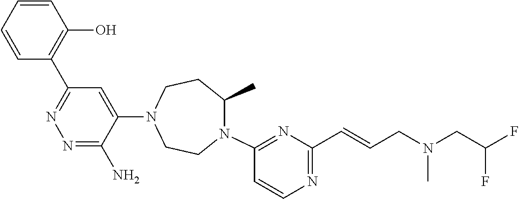 [SMILES: C[C@@H]1CCN(c2cc(-c3ccccc3O)nnc2N)CCN1c1ccnc(/C=C/CN(C)CC(F)F)n1] | o-{5-[(R)-4-(2-{3-[3- (methoxymethyl)-4-morpholinyl]-1- propynyl}-4-pyrimidinyl)-5-methyl- 1,4-diazepan-1-yl]-6-amino-3- pyridazinyl}phenol |
| P-164 | 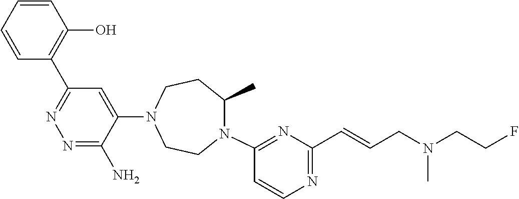 [SMILES: C[C@@H]1CCN(c2cc(-c3ccccc3O)nnc2N)CCN1c1ccnc(/C=C/CN(C)CCF)n1] | o-{5-[(R)-4-(2-{3-[(2-fluoroethyl)-N- methylamino]-1-propenyl}-4- pyrimidinyl)-5-methyl-1,4-diazepan- 1-yl]-6-amino-3-pyridazinyl}phenol |
| P-165 | 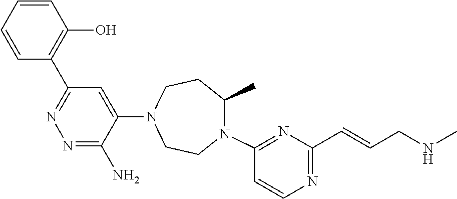 [SMILES: CNC/C=C/c1nccc(N2CCN(c3cc(-c4ccccc4O)nnc3N)CC[C@H]2C)n1] | o-{5-[(R)-5-methyl-4-{2-[3- (methylamino)-1-propenyl]-4- pyrimidinyl}-1,4-diazepan-1-yl]-6- amino-3-pyridazinyl}phenol |
| P-166 | 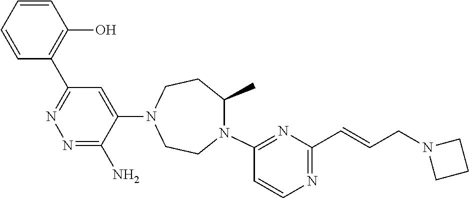 [SMILES: C[C@@H]1CCN(c2cc(-c3ccccc3O)nnc2N)CCN1c1ccnc(/C=C/CN2CCC2)n1] | o-{5-[(R)-4-{2-[3-(1-azetidinyl)-1- propenyl]-4-pyrimidinyl}-5-methyl- 1,4-diazepan-1-yl]-6-amino-3- pyridazinyl}phenol |
| P-167 | 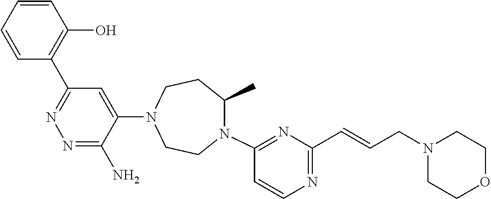 [SMILES: C[C@@H]1CCN(c2cc(-c3ccccc3O)nnc2N)CCN1c1ccnc(/C=C/CN2CCOCC2)n1] | o-(5-{(R)-5-methyl-4-[2-(3- morpholino-1-propenyl)-4- pyrimidinyl]-1,4-diazepan-1-yl}-6- amino-3-pyridazinyl)phenol |
| P-168 | 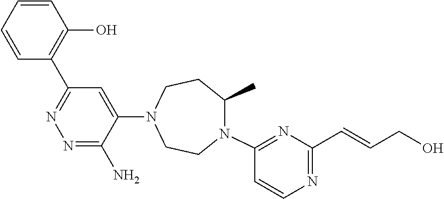 [SMILES: C[C@@H]1CCN(c2cc(-c3ccccc3O)nnc2N)CCN1c1ccnc(/C=C/CO)n1] | o-(5-{(R)-4-[2-(3-hydroxy-1- propenyl)-4-pyrimidinyl]-5-methyl- 1,4-diazepan-1-yl}-6-amino-3- pyridazinyl)phenol |
| P-169 | 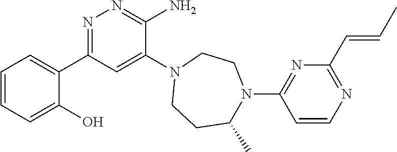 [SMILES: C/C=C/c1nccc(N2CCN(c3cc(-c4ccccc4O)nnc3N)CC[C@H]2C)n1] | o-(5-{(R)-5-methyl-4-[2-(1- propenyl)-4-pyrimidinyl]-1,4- diazepan-1-yl}-6-amino-3- pyridazinyl)phenol |
| P-170 | 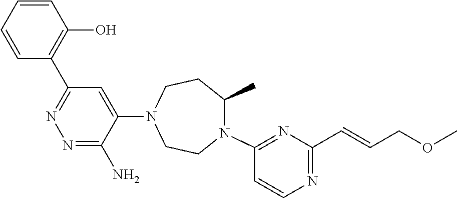 [SMILES: COC/C=C/c1nccc(N2CCN(c3cc(-c4ccccc4O)nnc3N)CC[C@H]2C)n1] | o-(5-{(R)-4-[2-(3-methoxy-1- propenyl)-4-pyrimidinyl]-5-methyl- 1,4-diazepan-1-yl}-6-amino-3- pyridazinyl)phenol |
| P-171 | 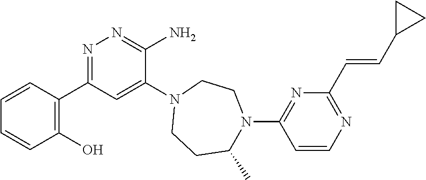 [SMILES: C[C@@H]1CCN(c2cc(-c3ccccc3O)nnc2N)CCN1c1ccnc(/C=C/C2CC2)n1] | o-(5-{(R)-4-[2-(2- cyclopropylethenyl)-4-pyrimidinyl]- 5-methyl-1,4-diazepan-1-yl}-6- amino-3-pyridazinyl)phenol |
| P-172 | 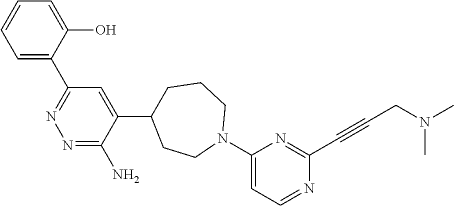 [SMILES: CN(C)CC#Cc1nccc(N2CCCC(c3cc(-c4ccccc4O)nnc3N)CC2)n1] | o-[6-amino-5-(1-{2-[3- (dimethylamino)-1-propynyl]-4- pyrimidinyl}-4-azepanyl)-3- pyridazinyl]phenol |
| P-173 | 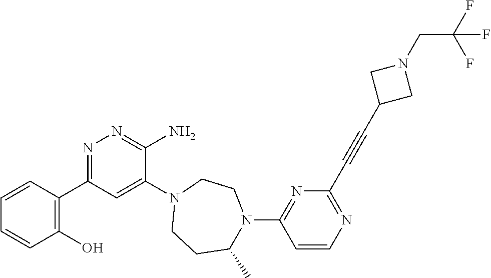 [SMILES: C[C@@H]1CCN(c2cc(-c3ccccc3O)nnc2N)CCN1c1ccnc(C#CC2CN(CC(F)(F)F)C2)n1] | o-{5-[(R)-5-methyl-4-(2-{2-[1-(2,2,2- trifluoroethyl)-3-azetidinyl]ethynyl}- 4-pyrimidinyl)-1,4-diazepan-1-yl]-6- amino-3-pyridazinyl}phenol |
| P-174 | 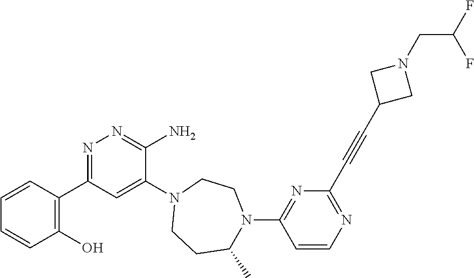 [SMILES: C[C@@H]1CCN(c2cc(-c3ccccc3O)nnc2N)CCN1c1ccnc(C#CC2CN(CC(F)F)C2)n1] | o-{5-[(R)-4-(2-{2-[1-(2,2- difluoroethyl)-3-azetidinyl]ethynyl}- 4-pyrimidinyl)-5-methyl-1,4- diazepan-1-yl]-6-amino-3- pyridazinyl}phenol |
| P-175 | 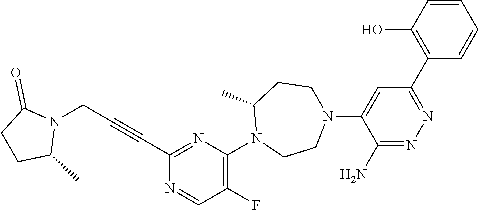 [SMILES: C[C@@H]1CCC(=O)N1CC#Cc1ncc(F)c(N2CCN(c3cc(-c4ccccc4O)nnc3N)CC[C@H]2C)n1] | (R)-1-[3-(4-{(R)-4-[3-amino-6-(o- hydroxyphenyl)-4-pyridazinyl]-7- methyl-1,4-diazepan-1-yl}-5-fluoro- 2-pyrimidinyl)-2-propynyl]-5-methyl- 2-pyrrolidinone |
| P-176 | 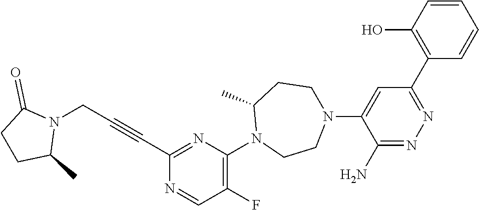 [SMILES: C[C@@H]1CCN(c2cc(-c3ccccc3O)nnc2N)CCN1c1nc(C#CCN2C(=O)CC[C@@H]2C)ncc1F] | (S)-1-[3-(4-{(R)-4-[3-amino-6-(o- hydroxyphenyl)-4-pyridazinyl]-7- methyl-1,4-diazepan-1-yl}-5-fluoro- 2-pyrimidinyl)-2-propynyl]-5-methyl- 2-pyrrolidinone |
| P-177 | 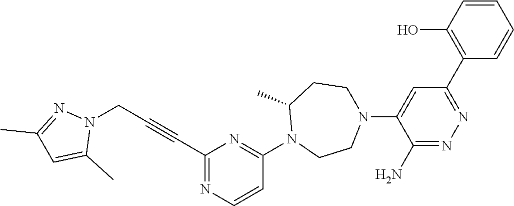 [SMILES: Cc1cc(C)n(CC#Cc2nccc(N3CCN(c4cc(-c5ccccc5O)nnc4N)CC[C@H]3C)n2)n1] | o-{5-[(R)-4-{2-[3-(3,5-dimethyl-1- pyrazolyl)-1-propynyl]-4- pyrimidinyl}-5-methyl-1,4-diazepan- 1-yl]-6-amino-3-pyridazinyl}phenol |
| P-178 | 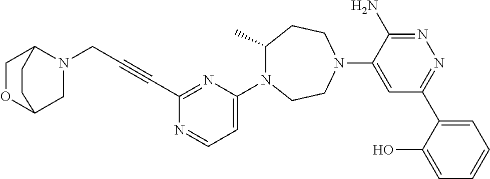 [SMILES: C[C@@H]1CCN(c2cc(-c3ccccc3O)nnc2N)CCN1c1ccnc(C#CCN2CC3CCC2CO3)n1] | o-{5-[(R)-5-methyl-4-(2-{3-(2-oxa-5- azabicyclo[2.2.2]oct-5-yl)-1- propynyl}-4-pyrimidinyl)-1,4- diazepan-1-yl]-6-amino-3- pyridazinyl}phenol |
| P-179 | 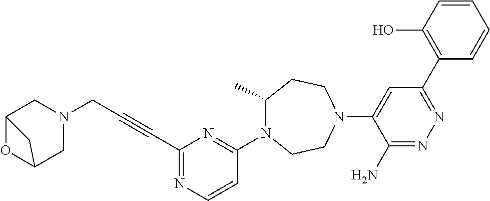 [SMILES: C[C@@H]1CCN(c2cc(-c3ccccc3O)nnc2N)CCN1c1ccnc(C#CCN2CC3CC(C2)O3)n1] | o-{5-[(R)-5-methyl-4-(2-{3-(6-oxa-3- azabicyclo[3.1.1]hept-3-yl)-1- propynyl}-4-pyrimidinyl)-1,4- diazepan-1-yl]-6-amino-3- pyridazinyl}phenol |
| P-180 | 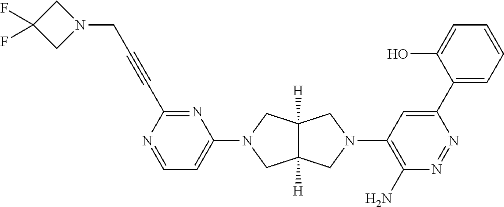 [SMILES: Nc1nnc(-c2ccccc2O)cc1N1C[C@H]2CN(c3ccnc(C#CCN4CC(F)(F)C4)n3)C[C@H]2C1] | o-{5-[(1R,5S)-7-{2-[3-(3,3-difluoro- 1-azetidinyl)-1-propynyl]-4- pyrimidinyl}-3,7- diazabicyclo[3.3.0]oct-3-yl]-6-amino- 3-pyridazinyl}phenol |
| P-181 | 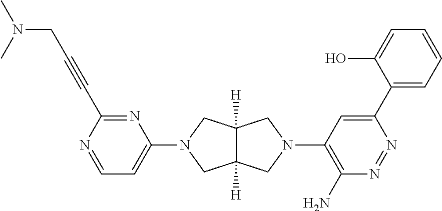 [SMILES: CN(C)CC#Cc1nccc(N2C[C@@H]3CN(c4cc(-c5ccccc5O)nnc4N)C[C@@H]3C2)n1] | o-{5-[(1R,5S)-7-{2-[3- (dimethylamino)-1-propynyl]-4- pyrimidinyl}-3,7- diazabicyclo[3.3.0]oct-3-yl]-6-amino- 3-pyridazinyl}phenol |
| P-182 | 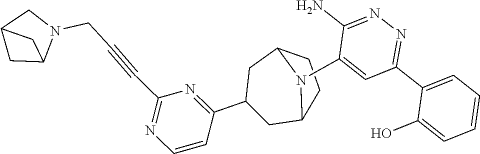 [SMILES: Nc1nnc(-c2ccccc2O)cc1N1C2CCC1CC(c1ccnc(C#CCN3CC4CC3C4)n1)C2] | o-{6-amino-5-[3-(2-{3-(2- azabicyclo[2.1.1]hex-2-yl)-1- propynyl}-4-pyrimidinyl)-3,8- diazabicyclo[3.2.1]oct-8-yl]-3- pyridazinyl}phenol |
| P-183 | 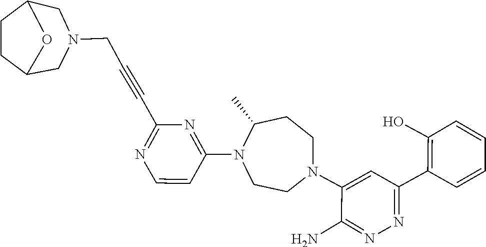 [SMILES: C[C@@H]1CCN(c2cc(-c3ccccc3O)nnc2N)CCN1c1ccnc(C#CCN2CC3CCC(C2)O3)n1] | o-{5-[(R)-5-methyl-4-(2-{3-(8-oxa-3- azabicyclo[3.2.1]oct-3-yl)-1- propynyl}-4-pyrimidinyl)-1,4- diazepan-1-yl]-6-amino-3- pyridazinyl}phenol |
| P-184 | 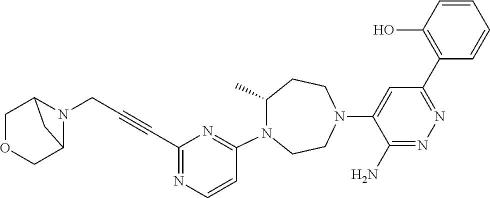 [SMILES: C[C@@H]1CCN(c2cc(-c3ccccc3O)nnc2N)CCN1c1ccnc(C#CCN2C3COCC2C3)n1] | o-{5-[(R)-5-methyl-4-(2-{3-(3-oxa-6- azabicyclo[3.1.1]hept-6-yl)-1- propynyl}-4-pyrimidinyl)-1,4- diazepan-1-yl]-6-amino-3- pyridazinyl}phenol |
| P-185 | 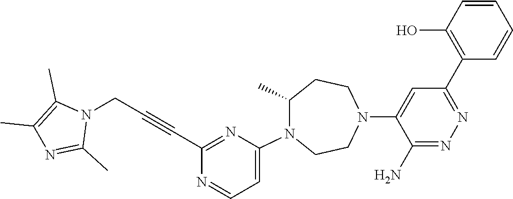 [SMILES: Cc1nc(C)n(CC#Cc2nccc(N3CCN(c4cc(-c5ccccc5O)nnc4N)CC[C@H]3C)n2)c1C] | o-{5-[(R)-5-methyl-4-(2-[3-(2,4,5- trimethyl-1-imidazolyl)-1-propynyl]- 4-pyrimidinyl}-1,4-diazepan-1-yl]-6- amino-3-pyridazinyl}phenol |
| P-186 | 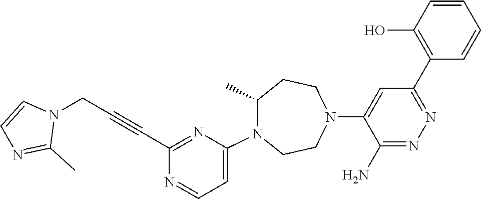 [SMILES: Cc1nccn1CC#Cc1nccc(N2CCN(c3cc(-c4ccccc4O)nnc3N)CC[C@H]2C)n1] | o-{5-[(R)-5-methyl-4-{2-[3-(2- methyl-1-imidazolyl)-1-propynyl]-4- pyrimidinyl}-1,4-diazepan-1-yl]-6- amino-3-pyridazinyl}phenol |
| P-187 | 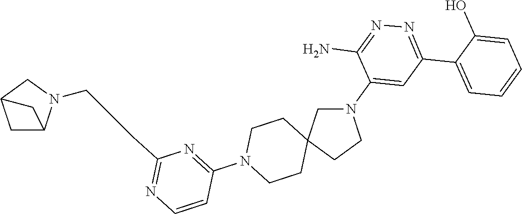 [SMILES: Nc1nnc(-c2ccccc2O)cc1N1CCC2(CCN(c3ccnc(CCCN4CC5CC4C5)n3)CC2)C1] | o-{6-amino-5-[8-(2-{3-(2- azabicyclo[2.1.1]hex-2-yl)-1- propynyl}-4-pyrimidinyl)-2,8-diaza- 2-spiro[4.5]decyl]-3- pyridazinyl}phenol |
| P-188 | 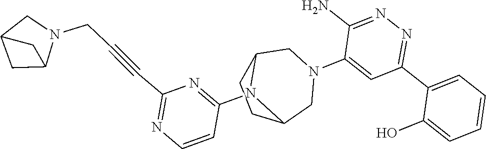 [SMILES: Nc1nnc(-c2ccccc2O)cc1N1CC2CCC(C1)N2c1ccnc(C#CCN2CC3CC2C3)n1] | o-{6-amino-5-[8-(2-{3-(2- azabicyclo[2.1.1]hex-2-yl)-1- propynyl}-4-pyrimidinyl)-3,8- diazabicyclo[3.2.1]oct-3-yl]-3- pyridazinyl}phenol |
| P-189 | 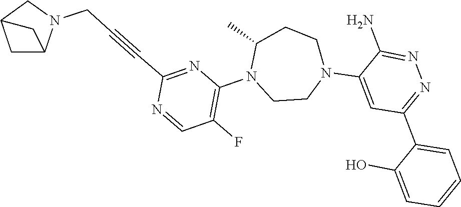 [SMILES: C[C@@H]1CCN(c2cc(-c3ccccc3O)nnc2N)CCN1c1nc(C#CCN2CC3CC2C3)ncc1F] | o-{5-[(R)-4-(2-{3-(2- azabicyclo[2.1.1]hex-2-yl)-1- propynyl}-5-fluoro-4-pyrimidinyl)-5- methyl-1,4-diazepan-1-yl]-6-amino-3- pyridazinyl}phenol |
| P-190 | 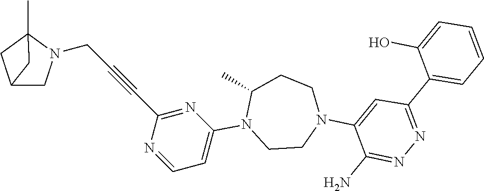 [SMILES: C[C@@H]1CCN(c2cc(-c3ccccc3O)nnc2N)CCN1c1ccnc(C#CCN2CC3CC2(C)C3)n1] | o-{5-[(R)-5-methyl-4-(2-{3-(1- methyl-2-azabicyclo[2.1.1]hex-2-yl)- 1-propynyl)-4-pyrimidinyl)-1,4- diazepan-1-yl]-6-amino-3- pyridazinyl}phenol |
| P-191 | 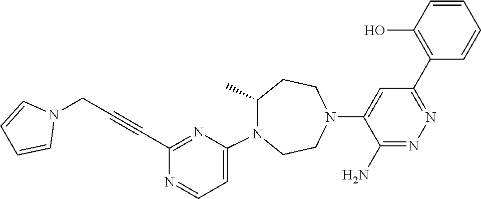 [SMILES: C[C@@H]1CCN(c2cc(-c3ccccc3O)nnc2N)CCN1c1ccnc(C#CCn2cccc2)n1] | o-{5-[(R)-5-methyl-4-{2-[3-(1- pyrrolyl)-1-propynyl]-4-pyrimidinyl}- 1,4-diazepan-1-yl]-6-amino-3- pyridazinyl}phenol |
| P-192 | 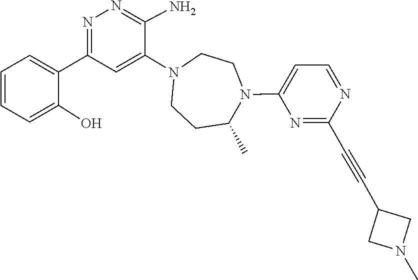 [SMILES: C[C@@H]1CCN(c2cc(-c3ccccc3O)nnc2N)CCN1c1ccnc(C#CC2CN(C)C2)n1] | o-{5-[(R)-5-methyl-4-{2-[2-(1- methyl-3-azetidinyl)ethynyl]-4- pyrimidinyl}-1,4-diazepan-1-yl]-6- amino-3-pyridazinyl}phenol |
| P-193 | 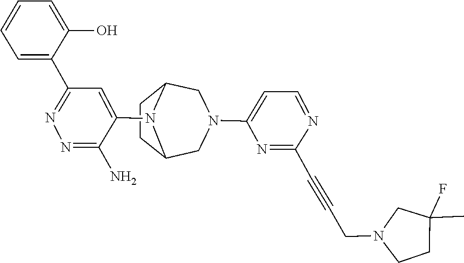 [SMILES: CC1(F)CCN(CC#Cc2nccc(N3CC4CCC(C3)N4c3cc(-c4ccccc4O)nnc3N)n2)C1] | o-[6-amino-5-(3-{2-[3-(3-fluoro-3- methyl-1-pyrrolidinyl)-1-propynyl]-4- pyrimidinyl}-3,8- diazabicyclo[3.2.1]oct-8-yl)-3- pyridazinyl]phenol |
| P-194 | 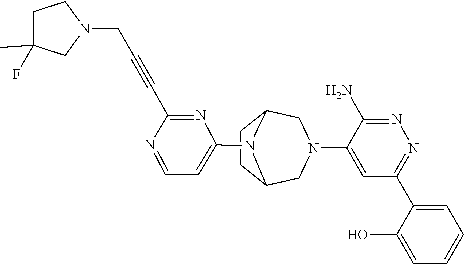 [SMILES: CC1(F)CCN(CC#Cc2nccc(N3C4CCC3CN(c3cc(-c5ccccc5O)nnc3N)C4)n2)C1] | o-[6-amino-5-(8-{2-[3-(3-fluoro-3- methyl-1-pyrrolidinyl)-1-propynyl]-4- pyrimidinyl}-3,8- diazabicyclo[3.2.1]oct-3-yl)-3- pyridazinyl]phenol |
| P-195 | 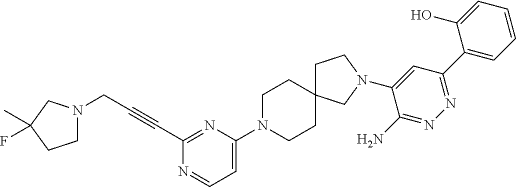 [SMILES: CC1(F)CCN(CC#Cc2nccc(N3CCC4(CC3)CCN(c3cc(-c5ccccc5O)nnc3N)C4)n2)C1] | o-[6-amino-5-(8-{2-[3-(3-fluoro-3- methyl-1-pyrrolidinyl)-1-propynyl]-4- pyrimidinyl}-2,8-diaza-2- spiro[4.5]decyl)-3-pyridazinyl]phenol |
| P-196 | 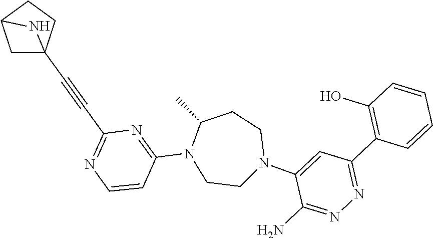 [SMILES: C[C@@H]1CCN(c2cc(-c3ccccc3O)nnc2N)CCN1c1ccnc(C#CC23CCC(C2)N3)n1] | o-{5-[(R)-4-(2-{2-(5- azabicyclo[2.1.1]hex-1-yl)ethynyl}-4- pyrimidinyl)-5-methyl-1,4-diazepan- 1-yl]-6-amino-3-pyridazinyl}phenol |
| P-197 | 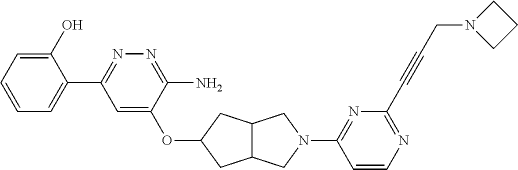 [SMILES: Nc1nnc(-c2ccccc2O)cc1OC1CC2CN(c3ccnc(C#CCN4CCC4)n3)CC2C1] | o-[6-amino-5-(3-{2-[3-(1-azetidinyl)- 1-propynyl]-4-pyrimidinyl}-3- azabicyclo[3.3.0]oct-7-yloxy)-3- pyridazinyl]phenol |
| P-198 | 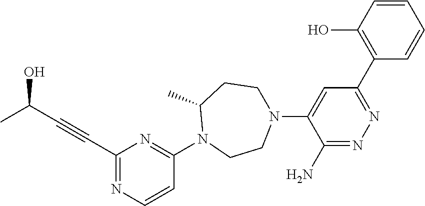 [SMILES: C[C@@H]1CCN(c2cc(-c3ccccc3O)nnc2N)CCN1c1ccnc(C#C[C@@H](C)O)n1] | o-{5-[(R)-4-{2-[(R)-3-hydroxy-1- butynyl]-4-pyrimidinyl}-5-methyl- 1,4-diazepan-1-yl]-6-amino-3- pyridazinyl}phenol |
| P-199 | 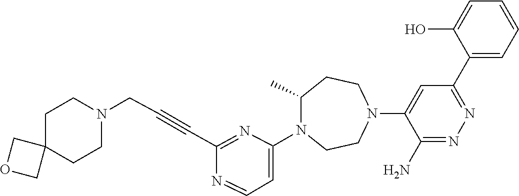 [SMILES: C[C@@H]1CCN(c2cc(-c3ccccc3O)nnc2N)CCN1c1ccnc(C#CCN2CCC3(CC2)COC3)n1] | o-{5-[(R)-5-methyl-4-(2-{3-(2-oxa-7- aza-7-spiro[3.5]nonyl)-1-propynyl}- 4-pyrimidinyl)-1,4-diazepan-1-yl]-6- amino-3-pyridazinyl}phenol |
| P-200 | 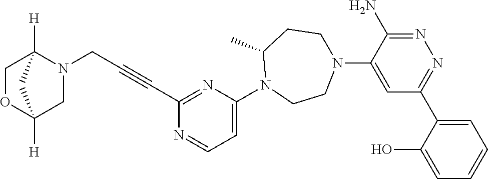 [SMILES: C[C@@H]1CCN(c2cc(-c3ccccc3O)nnc2N)CCN1c1ccnc(C#CCN2C[C@@H]3C[C@H]2CO3)n1] | o-{5-[(R)-4-[2-(3-{(1S,4S)-2-oxa-5- azabicyclo[2.2.1]hept-5-yl}-1- propynyl)-4-pyrimidinyl]-5-methyl- 1,4-diazepan-1-yl]-6-amino-3- pyridazinyl}phenol |
| P-201 | 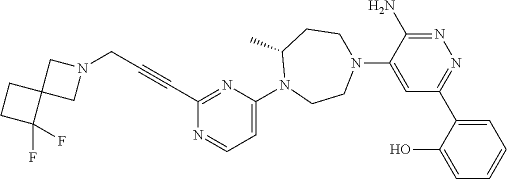 [SMILES: C[C@@H]1CCN(c2cc(-c3ccccc3O)nnc2N)CCN1c1ccnc(C#CCN2CC3(CCC3(F)F)C2)n1] | o-{5-[(R)-4-(2-{3-(5,5-difluoro-2- aza-2-spiro[3.3]heptyl)-1-propynyl}- 4-pyrimidinyl)-5-methyl-1,4- diazepan-1-yl]-6-amino-3- pyridazinyl}phenol |
| P-202 | 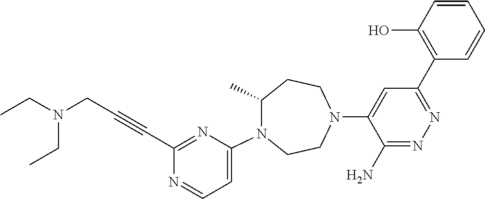 [SMILES: CCN(CC)CC#Cc1nccc(N2CCN(c3cc(-c4ccccc4O)nnc3N)CC[C@H]2C)n1] | o-{5-[(R)-4-{2-[3-(diethylamino)-1- propynyl]-4-pyrimidinyl}-5-methyl- 1,4-diazepan-1-yl]-6-amino-3- pyridazinyl}phenol |
| P-203 | 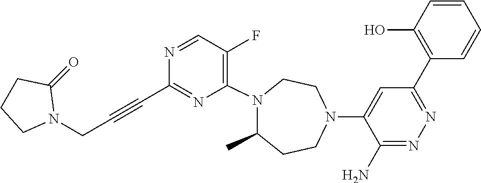 [SMILES: C[C@@H]1CCN(c2cc(-c3ccccc3O)nnc2N)CCN1c1nc(C#CCN2CCCC2=O)ncc1F] | 1-[3-(4-{(R)-4-[3-amino-6-(o- hydroxyphenyl)-4-pyridazinyl]-7- methyl-1,4-diazepan-1-yl}-5-fluoro- 2-pyrimidinyl)-2-propynyl]-2- pyrrolidinone |
| P-204 | 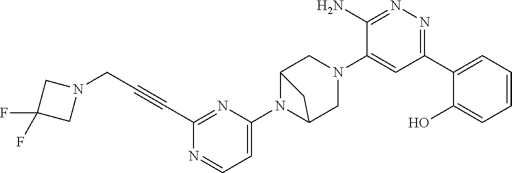 [SMILES: Nc1nnc(-c2ccccc2O)cc1N1CC2CC(C1)N2c1ccnc(C#CCN2CC(F)(F)C2)n1] | o-[6-amino-5-(6-{2-[3-(3,3-difluoro- 1-azetidinyl)-1-propynyl]-4- pyrimidinyl}-3,6- diazabicyclo[3.1.1]hept-3-yl)-3- pyridazinyl]phenol |
| P-205 | 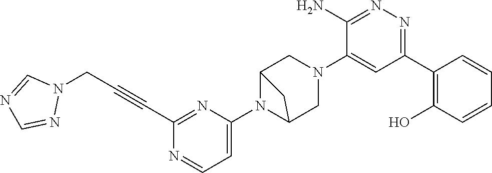 [SMILES: Nc1nnc(-c2ccccc2O)cc1N1CC2CC(C1)N2c1ccnc(C#CCn2cncn2)n1] | o-[6-amino-5-(6-{2-[3-(1H-1,2,4- triazol-1-yl)-1-propynyl]-4- pyrimidinyl}-3,6- diazabicyclo[3.1.1]hept-3-yl)-3- pyridazinyl]phenol |
| P-206 | 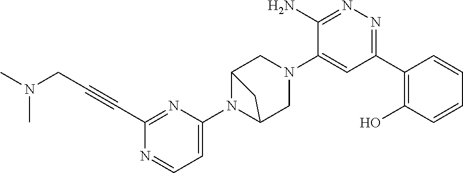 [SMILES: CN(C)CC#Cc1nccc(N2C3CC2CN(c2cc(-c4ccccc4O)nnc2N)C3)n1] | o-[6-amino-5-(6-{2-[3- (dimethylamino)-1-propynyl]-4- pyrimidinyl}-3,6- diazabicyclo[3.1.1]hept-3-yl)-3- pyridazinyl]phenol |
| P-207 | 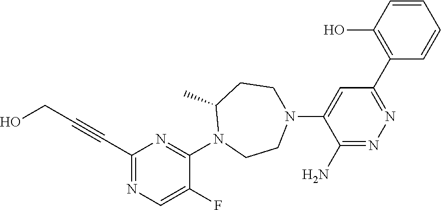 [SMILES: C[C@@H]1CCN(c2cc(-c3ccccc3O)nnc2N)CCN1c1nc(C#CCO)ncc1F] | o-(5-{(R)-4-[5-fluoro-2-(3-hydroxy- 1-propynyl)-4-pyrimidinyl]-5-methyl- 1,4-diazepan-1-yl}-6-amino-3- pyridazinyl)phenol |
| P-208 | 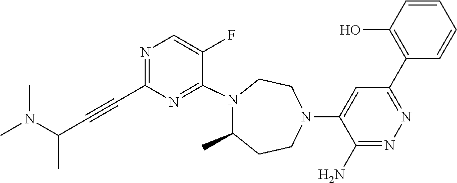 [SMILES: CC(C#Cc1ncc(F)c(N2CCN(c3cc(-c4ccccc4O)nnc3N)CC[C@H]2C)n1)N(C)C] | o-{5-[(R)-4-{2-[3-(dimethylamino)-1- butynyl]-5-fluoro-4-pyrimidinyl}-5- methyl-1,4-diazepan-1-yl]-6-amino-3- pyridazinyl}phenol |
| P-209 | 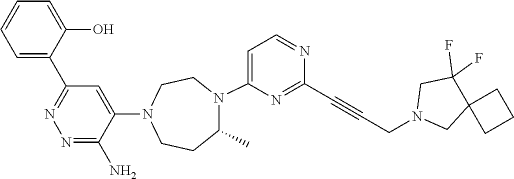 [SMILES: C[C@@H]1CCN(c2cc(-c3ccccc3O)nnc2N)CCN1c1ccnc(C#CCN2CC(F)(F)C3(CCC3)C2)n1] | o-{5-[(R)-4-(2-{3-(8,8-difluoro-6- aza-6-spiro[3.4]octyl)-1-propynyl}-4- pyrimidinyl)-5-methyl-1,4-diazepan- 1-yl]-6-amino-3-pyridazinyl}phenol |
| P-210 | 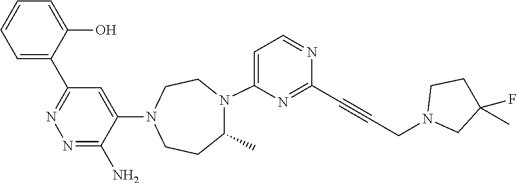 [SMILES: C[C@@H]1CCN(c2cc(-c3ccccc3O)nnc2N)CCN1c1ccnc(C#CCN2CCC(C)(F)C2)n1] | o-{5-[(R)-4-{2-[3-(3-fluoro-3-methyl- 1-pyrrolidinyl)-1-propynyl]-4- pyrimidinyl}-5-methyl-1,4-diazepan- 1-yl]-6-amino-3-pyridazinyl}phenol |
| P-211 | 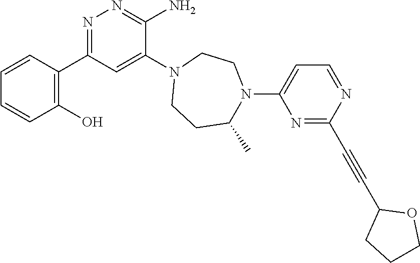 [SMILES: C[C@@H]1CCN(c2cc(-c3ccccc3O)nnc2N)CCN1c1ccnc(C#CC2CCCO2)n1] | o-{5-[(R)-5-methyl-4-{2-[2- (tetrahydro-2-furyl)ethynyl]-4- pyrimidinyl}-1,4-diazepan-1-yl]-6- amino-3-pyridazinyl}phenol |
| P-212 | 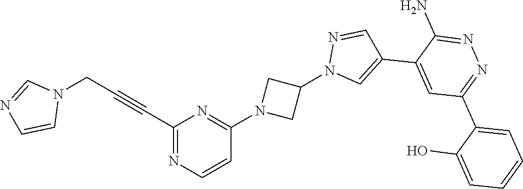 [SMILES: Nc1nnc(-c2ccccc2O)cc1-c1cnn(C2CN(c3ccnc(C#CCn4ccnc4)n3)C2)c1] | o-{6-amino-5-[1-(1-{2-[3-(1- imidazolyl)-1-propynyl]-4- pyrimidinyl}-3-azetidinyl)-4- pyrazolyl]-3-pyridazinyl}phenol |
| P-213 | 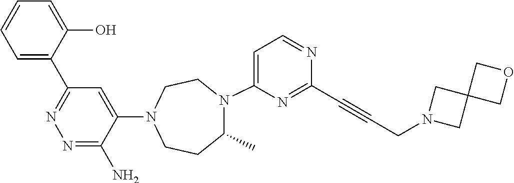 [SMILES: C[C@@H]1CCN(c2cc(-c3ccccc3O)nnc2N)CCN1c1ccnc(C#CCN2CC3(COC3)C2)n1] | o-{5-[(R)-5-methyl-4-(2-{3-(2-oxa-6- aza-6-spiro[3.3]heptyl)-1-propynyl}- 4-pyrimidinyl)-1,4-diazepan-1-yl]-6- amino-3-pyridazinyl}phenol |
| P-214 | 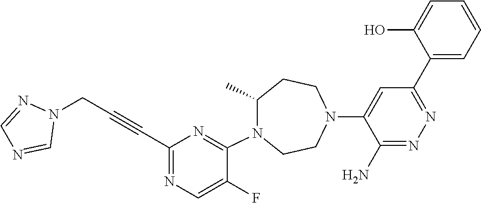 [SMILES: C[C@@H]1CCN(c2cc(-c3ccccc3O)nnc2N)CCN1c1nc(C#CCn2cncn2)ncc1F] | o-{5-[(R)-4-{5-fluoro-2-[3-(1H-1,2,4- triazol-1-yl)-1-propynyl]-4- pyrimidinyl}-5-methyl-1,4-diazepan- 1-yl]-6-amino-3-pyridazinyl}phenol |
| P-215 | 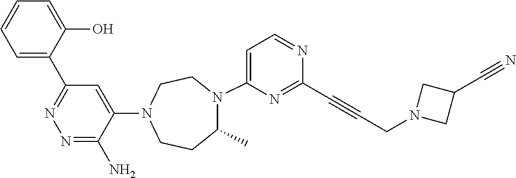 [SMILES: C[C@@H]1CCN(c2cc(-c3ccccc3O)nnc2N)CCN1c1ccnc(C#CCN2CC(C#N)C2)n1] | 1-[3-(4-{(R)-4-[3-amino-6-(o- hydroxyphenyl)-4-pyridazinyl]-7- methyl-1,4-diazepan-1-yl}-2- pyrimidinyl)-2-propynyl]-3- azetidinecarbonitrile |
| P-216 | 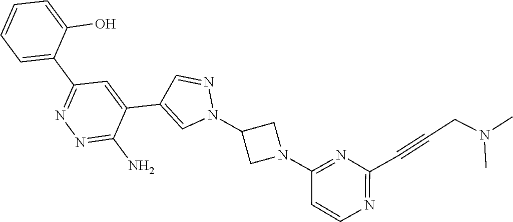 [SMILES: CN(C)CC#Cc1nccc(N2CC(n3cc(-c4cc(-c5ccccc5O)nnc4N)cn3)C2)n1] | o-{6-amino-5-[1-(1-{2-[3- (dimethylamino)-1-propynyl]-4- pyrimidinyl}-3-azetidinyl)-4- pyrazolyl]-3-pyridazinyl}phenol |
| P-217 | 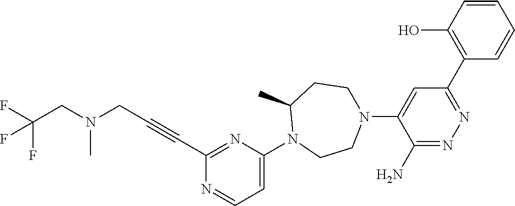 [SMILES: C[C@H]1CCN(c2cc(-c3ccccc3O)nnc2N)CCN1c1ccnc(C#CCN(C)CC(F)(F)F)n1] | o-(5-[(S)-4-(2-{3-[N-methyl(2,2,2- trifluoroethyl)amino]-1-propynyl}-4- pyrimidinyl)-5-methyl-1,4-diazepan- 1-yl]-6-amino-3-pyridazinyl}phenol |
| P-218 | 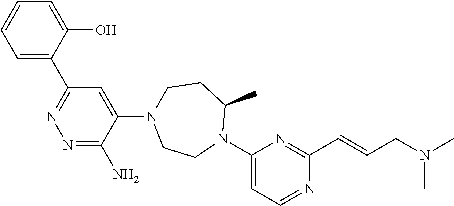 [SMILES: C[C@@H]1CCN(c2cc(-c3ccccc3O)nnc2N)CCN1c1ccnc(/C=C/CN(C)C)n1] | o-{5-[(R)-4-{2-[(E)-3- (dimethylamino)-1-propenyl]-4- pyrimidinyl}-5-methyl-1,4-diazepan- 1-yl]-6-amino-3-pyridazinyl}phenol |
| P-219 | 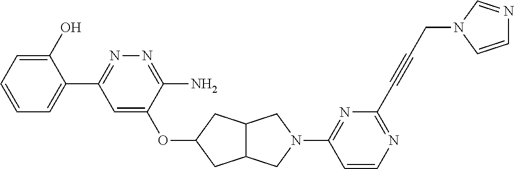 [SMILES: Nc1nnc(-c2ccccc2O)cc1OC1CC2CN(c3ccnc(C#CCn4ccnc4)n3)CC2C1] | o-[6-amino-5-(3-{2-[3-(1- imidazolyl)-1-propynyl]-4- pyrimidinyl}-3-azabicyclo[3.3.0]oct- 7-yloxy)-3-pyridazinyl]phenol |
| P-220 | 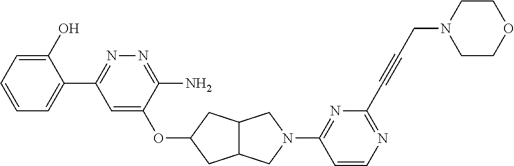 [SMILES: Nc1nnc(-c2ccccc2O)cc1OC1CC2CN(c3ccnc(C#CCN4CCOCC4)n3)CC2C1] | o-(6-amino-5-{3-[2-(3-morpholino-1- propynyl)-4-pyrimidinyl]-3- azabicyclo[3.3.0]oct-7-yloxy}-3- pyridazinyl)phenol |
| P-221 | 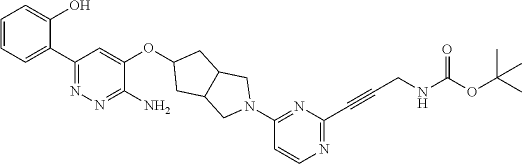 [SMILES: CC(C)(C)OC(=O)NCC#Cc1nccc(N2CC3CC(Oc4cc(-c5ccccc5O)nnc4N)CC3C2)n1] | 4-(3-{2-[3-(tert- butoxycarbonylamino)-1-propynyl]- 4-pyrimidinyl}-3- azabicyclo[3.3.0]oct-7-yloxy)-6-(o- hydroxyphenyl)-3-pyridazinamine |
| P-222 | 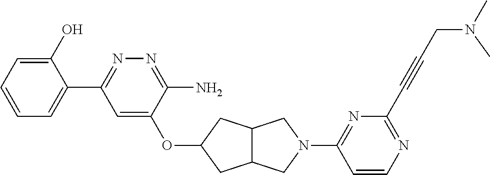 [SMILES: CN(C)CC#Cc1nccc(N2CC3CC(Oc4cc(-c5ccccc5O)nnc4N)CC3C2)n1] | o-[6-amino-5-(3-{2-[3- (dimethylamino)-1-propynyl]-4- pyrimidinyl}-3-azabicyclo[3.3.0]oct- 7-yloxy)-3-pyridazinyl]phenol |
| P-223 | 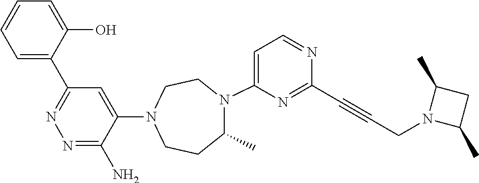 [SMILES: C[C@@H]1CCN(c2cc(-c3ccccc3O)nnc2N)CCN1c1ccnc(C#CCN2[C@H](C)C[C@@H]2C)n1] | o-{5-[(R)-4-(2-{3-[(2R,4S)-2,4- dimethyl-1-azetidinyl]-1-propynyl}- 4-pyrimidinyl)-5-methyl-1,4- diazepan-1-yl]-6-amino-3- pyridazinyl}phenol |
| P-224 | 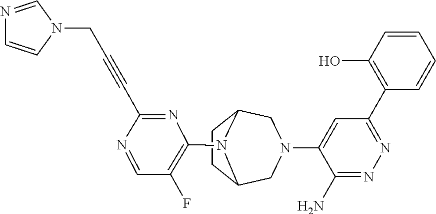 [SMILES: Nc1nnc(-c2ccccc2O)cc1N1CC2CCC(C1)N2c1nc(C#CCn2ccnc2)ncc1F] | o-[6-amino-5-(8-{5-fluoro-2-[3-(1- imidazolyl)-1-propynyl]-4- pyrimidinyl}-3,8- diazabicyclo[3.2.1]oct-3-yl)-3- pyridazinyl]phenol |
| P-225 | 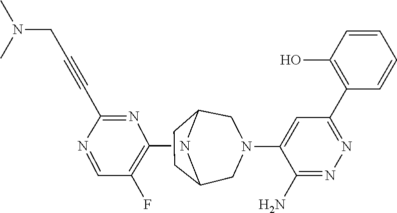 [SMILES: CN(C)CC#Cc1ncc(F)c(N2C3CCC2CN(c2cc(-c4ccccc4O)nnc2N)C3)n1] | o-[6-amino-5-(8-{2-[3- (dimethylamino)-1-propynyl]-5- fluoro-4-pyrimidinyl}-3,8- diazabicyclo[3.2.1]oct-3-yl)-3- pyridazinyl]phenol |
| P-226 | 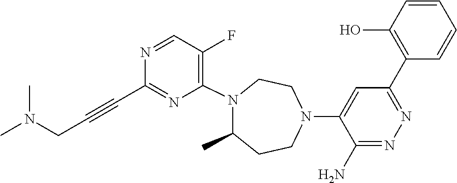 [SMILES: C[C@@H]1CCN(c2cc(-c3ccccc3O)nnc2N)CCN1c1nc(C#CCN(C)C)ncc1F] | o-{5-[(R)-4-{2-[3-(dimethylamino)-1- propynyl]-5-fluoro-4-pyrimidinyl}-5- methyl-1,4-diazepan-1-yl]-6-amino-3- pyridazinyl}phenol |
| P-227 | 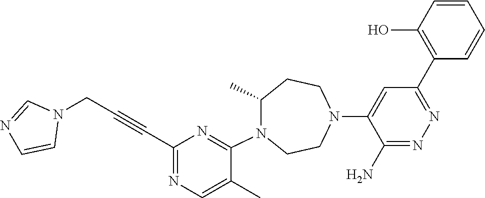 [SMILES: Cc1cnc(C#CCn2ccnc2)nc1N1CCN(c2cc(-c3ccccc3O)nnc2N)CC[C@H]1C] | o-(5-[(R)-4-(2-[3-(1-imidazolyl)-1- propynyl]-5-methyl-4-pyrimidinyl}- 5-methyl-1,4-diazepan-1-yl]-6-amino- 3-pyridazinyl}phenol |
| P-228 | 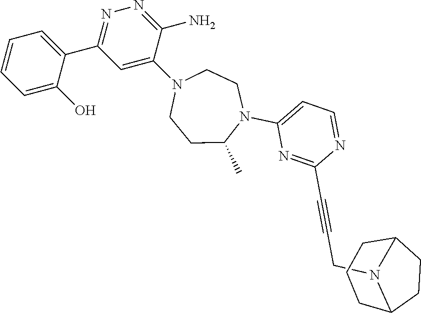 [SMILES: C[C@@H]1CCN(c2cc(-c3ccccc3O)nnc2N)CCN1c1ccnc(C#CCN2C3CCCC2CC3)n1] | o-{5-[(R)-4-(2-{3-(8- azabicyclo[3.2.1]oct-8-yl)-1- propynyl}-4-pyrimidinyl)-5-methyl- 1,4-diazepan-1-yl]-6-amino-3- pyridazinyl}phenol |
| P-229 | 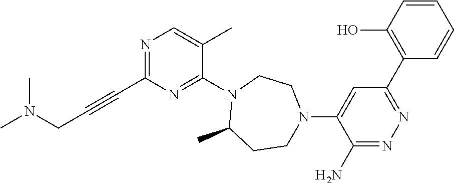 [SMILES: Cc1cnc(C#CCN(C)C)nc1N1CCN(c2cc(-c3ccccc3O)nnc2N)CC[C@H]1C] | o-{5-[(R)-4-{2-[3-(dimethylamino)-1- propynyl]-5-methyl-4-pyrimidinyl}- 5-methyl-1,4-diazepan-1-yl]-6-amino- 3-pyridazinyl}phenol |
| P-230 | 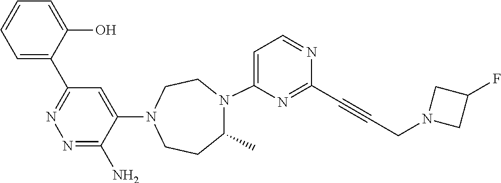 [SMILES: C[C@@H]1CCN(c2cc(-c3ccccc3O)nnc2N)CCN1c1ccnc(C#CCN2CC(F)C2)n1] | o-{5-[(R)-4-{2-[3-(3-fluoro-1- azetidinyl)-1-propynyl]-4- pyrimidinyl}-5-methyl-1,4-diazepan- 1-yl]-6-amino-3-pyridazinyl}phenol |
| P-231 | 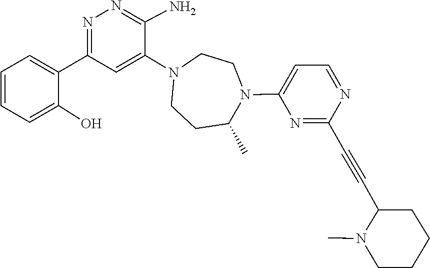 [SMILES: C[C@@H]1CCN(c2cc(-c3ccccc3O)nnc2N)CCN1c1ccnc(C#CC2CCCCN2C)n1] | o-{5-[(R)-5-methyl-4-{2-[2-(1- methyl-2-piperidyl)ethynyl]-4- pyrimidinyl}-1,4-diazepan-1-yl]-6- amino-3-pyridazinyl}phenol |
| P-232 | 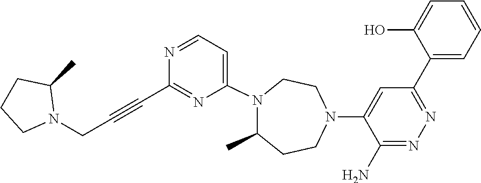 [SMILES: C[C@@H]1CCCN1CC#Cc1nccc(N2CCN(c3cc(-c4ccccc4O)nnc3N)CC[C@H]2C)n1] | o-{5-[(R)-4-(2-{3-[(R)-2-methyl-1- pyrrolidinyl]-1-propynyl}-4- pyrimidinyl)-5-methyl-1,4-diazepan- 1-yl]-6-amino-3-pyridazinyl}phenol |
| P-233 | 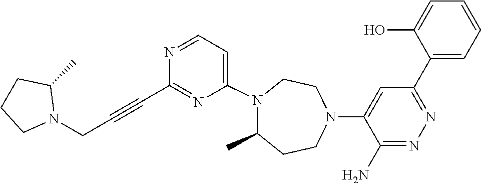 [SMILES: C[C@@H]1CCN(c2cc(-c3ccccc3O)nnc2N)CCN1c1ccnc(C#CCN2CCC[C@@H]2C)n1] | o-{5-[(R)-4-(2-{3-[(S)-2-methyl-1- pyrrolidinyl]-1-propynyl}-4- pyrimidinyl)-5-methyl-1,4-diazepan- 1-yl]-6-amino-3-pyridazinyl}phenol |
| P-234 | 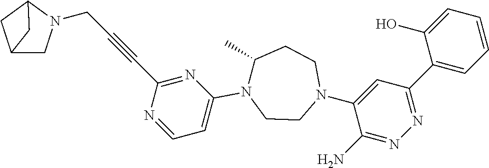 [SMILES: C[C@@H]1CCN(c2cc(-c3ccccc3O)nnc2N)CCN1c1ccnc(C#CCN2CC3CC2C3)n1] | o-{5-[(R)-4-(2-{3-(2- azabicyclo[2.1.1]hex-2-yl)-1- propynyl}-4-pyrimidinyl)-5-methyl- 1,4-diazepan-1-yl]-6-amino-3- pyridazinyl}phenol |
| P-235 | 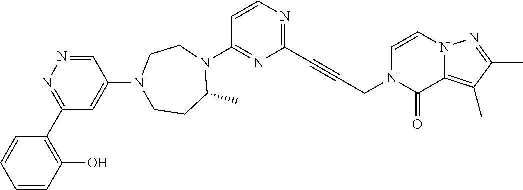 [SMILES: Cc1nn2ccn(CC#Cc3nccc(N4CCN(c5cnnc(-c6ccccc6O)c5)CC[C@H]4C)n3)c(=O)c2c1C] | 5-[3-(4-{(R)-4-[6-(o-hydroxyphenyl)- 4-pyridazinyl]-7-methyl-1,4- diazepan-1-yl}-2-pyrimidinyl)-2- propynyl]-2,3-dimethyl-1,5,7a-triaza- 4(5H)-indenone |
| P-236 | 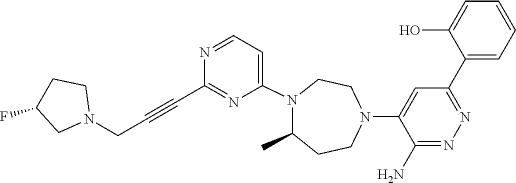 [SMILES: C[C@@H]1CCN(c2cc(-c3ccccc3O)nnc2N)CCN1c1ccnc(C#CCN2CC[C@@H](F)C2)n1] | o-{5-[(R)-4-(2-{3-[(R)-3-fluoro-1- pyrrolidinyl]-1-propynyl}-4- pyrimidinyl)-5-methyl-1,4-diazepan- 1-yl]-6-amino-3-pyridazinyl}phenol |
| P-237 | 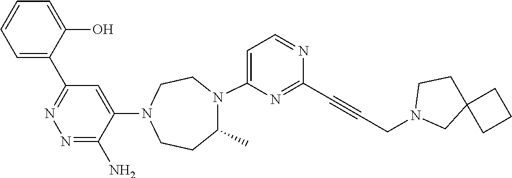 [SMILES: C[C@@H]1CCN(c2cc(-c3ccccc3O)nnc2N)CCN1c1ccnc(C#CCN2CCC3(CCC3)C2)n1] | o-{5-[(R)-4-(2-{3-(6-aza-6- spiro[3.4]octyl)-1-propynyl}-4- pyrimidinyl)-5-methyl-1,4-diazepan- 1-yl]-6-amino-3-pyridazinyl}phenol |
| P-238 | 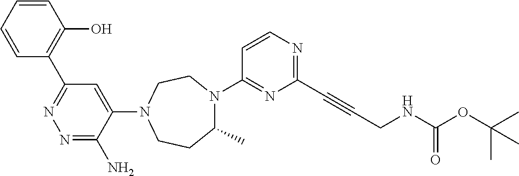 [SMILES: C[C@@H]1CCN(c2cc(-c3ccccc3O)nnc2N)CCN1c1ccnc(C#CCNC(=O)OC(C)(C)C)n1] | 4-[(R)-4-{2-[3-(tert- butoxycarbonylamino)-1-propynyl]- 4-pyrimidinyl}-5-methyl-1,4- diazepan-1-yl]-6-(o-hydroxyphenyl)- 3-pyridazinamine |
| P-239 | 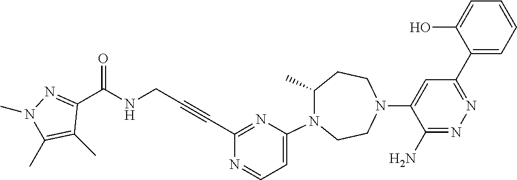 [SMILES: Cc1c(C(=O)NCC#Cc2nccc(N3CCN(c4cc(-c5ccccc5O)nnc4N)CC[C@H]3C)n2)nn(C)c1C] | N-[3-(4-{(R)-4-[3-amino-6-(o- hydroxyphenyl)-4-pyridazinyl]-7- methyl-1,4-diazepan-1-yl}-2- pyrimidinyl)-2-propynyl]-1-methyl- 4,5-dimethyl-3-pyrazolecarboxamide |
| P-240 | 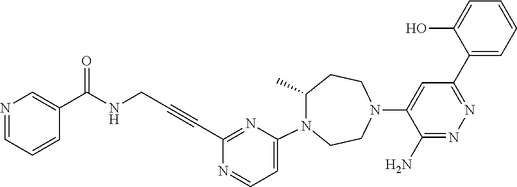 [SMILES: C[C@@H]1CCN(c2cc(-c3ccccc3O)nnc2N)CCN1c1ccnc(C#CCNC(=O)c2cccnc2)n1] | N-[3-(4-{(R)-4-[3-amino-6-(o- hydroxyphenyl)-4-pyridazinyl]-7- methyl-1,4-diazepan-1-yl}-2- pyrimidinyl)-2- propynyl]nicotinamide |
| P-241 | 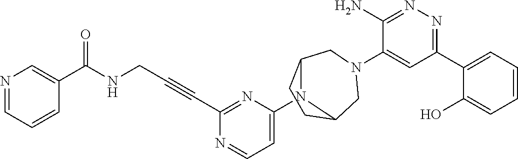 [SMILES: Nc1nnc(-c2ccccc2O)cc1N1CC2CCC(C1)N2c1ccnc(C#CCNC(=O)c2cccnc2)n1] | N-[3-(4-{3-[3-amino-6-(o- hydroxyphenyl)-4-pyridazinyl]-3,8- diazabicyclo[3.2.1]oct-8-yl}-2- pyrimidinyl)-2- propynyl]nicotinamide |
| P-242 | 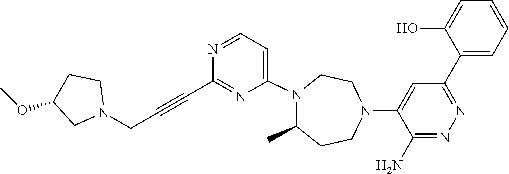 [SMILES: CO[C@@H]1CCN(CC#Cc2nccc(N3CCN(c4cc(-c5ccccc5O)nnc4N)CC[C@H]3C)n2)C1] | o-{5-[(R)-4-(2-{3-[(R)-3-methoxy-1- pyrrolidinyl]-1-propynyl}-4- pyrimidinyl)-5-methyl-1,4-diazepan- 1-yl]-6-amino-3-pyridazinyl}phenol |
| P-243 | 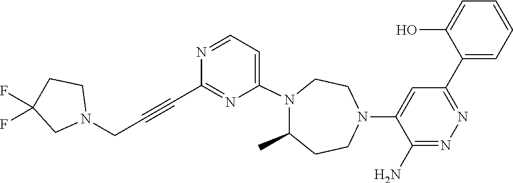 [SMILES: C[C@@H]1CCN(c2cc(-c3ccccc3O)nnc2N)CCN1c1ccnc(C#CCN2CCC(F)(F)C2)n1] | o-{5-[(R)-4-{2-[3-(3,3-difluoro-1- pyrrolidinyl)-1-propynyl]-4- pyrimidinyl}-5-methyl-1,4-diazepan- 1-yl]-6-amino-3-pyridazinyl}phenol |
| P-244 | 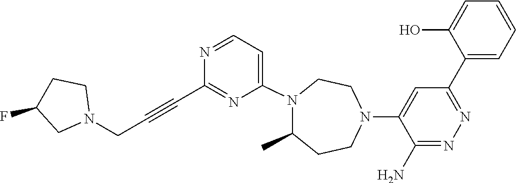 [SMILES: C[C@@H]1CCN(c2cc(-c3ccccc3O)nnc2N)CCN1c1ccnc(C#CCN2CC[C@H](F)C2)n1] | o-{5-[(R)-4-(2-{3-[(S)-3-fluoro-1- pyrrolidinyl]-1-propynyl}-4- pyrimidinyl)-5-methyl-1,4-diazepan- 1-yl]-6-amino-3-pyridazinyl}phenol |
| P-245 | 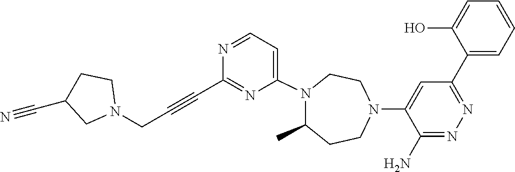 [SMILES: C[C@@H]1CCN(c2cc(-c3ccccc3O)nnc2N)CCN1c1ccnc(C#CCN2CCC(C#N)C2)n1] | 1-[3-(4-{(R)-4-[3-amino-6-(o- hydroxyphenyl)-4-pyridazinyl]-7- methyl-1,4-diazepan-1-yl}-2- pyrimidinyl)-2-propynyl]-3- pyrrolidinecarbonitrile |
| P-246 | 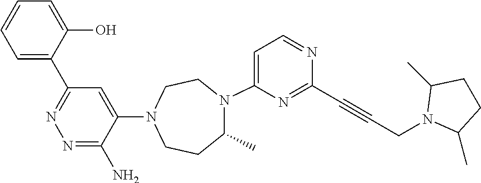 [SMILES: CC1CCC(C)N1CC#Cc1nccc(N2CCN(c3cc(-c4ccccc4O)nnc3N)CC[C@H]2C)n1] | o-{5-[(R)-4-{2-[3-(2,5-dimethyl-1- pyrrolidinyl)-1-propynyl]-4- pyrimidinyl}-5-methyl-1,4-diazepan- 1-yl]-6-amino-3-pyridazinyl}phenol |
| P-247 | 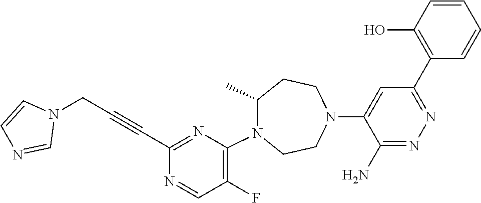 [SMILES: C[C@@H]1CCN(c2cc(-c3ccccc3O)nnc2N)CCN1c1nc(C#CCn2ccnc2)ncc1F] | o-{5-[(R)-4-{5-fluoro-2-[3-(1- imidazolyl)-1-propynyl]-4- pyrimidinyl}-5-methyl-1,4-diazepan- 1-yl]-6-amino-3-pyridazinyl}phenol |
| P-248 | 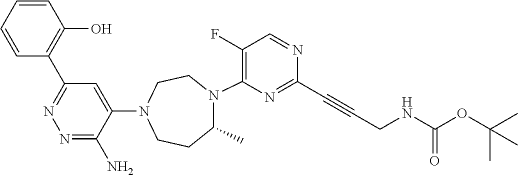 [SMILES: C[C@@H]1CCN(c2cc(-c3ccccc3O)nnc2N)CCN1c1nc(C#CCNC(=O)OC(C)(C)C)ncc1F] | 3-(4-{(R)-4-[3-amino-6-(o- hydroxyphenyl)-4-pyridazinyl]-7- methyl-1,4-diazepan-1-yl}-5-fluoro- 2-pyrimidinyl)-2-propynylamino-tert- butylformylate |
| P-249 | 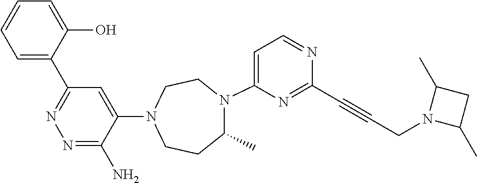 [SMILES: CC1CC(C)N1CC#Cc1nccc(N2CCN(c3cc(-c4ccccc4O)nnc3N)CC[C@H]2C)n1] | o-{5-[(R)-4-{2-[3-(2,4-dimethyl-1- azetidinyl)-1-propynyl]-4- pyrimidinyl}-5-methyl-1,4-diazepan- 1-yl]-6-amino-3-pyridazinyl}phenol |
| P-250 | 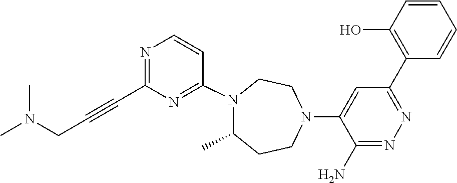 [SMILES: C[C@H]1CCN(c2cc(-c3ccccc3O)nnc2N)CCN1c1ccnc(C#CCN(C)C)n1] | o-{5-[(S)-4-{2-[3-(dimethylamino)-1- propynyl]-4-pyrimidinyl}-5-methyl- 1,4-diazepan-1-yl]-6-amino-3- pyridazinyl}phenol |
| P-251 | 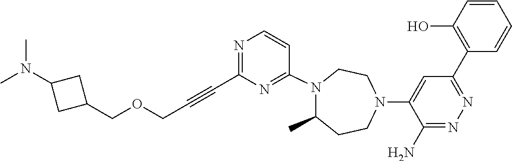 [SMILES: C[C@@H]1CCN(c2cc(-c3ccccc3O)nnc2N)CCN1c1ccnc(C#CCOCC2CC(N(C)C)C2)n1] | o-{5-[(R)-4-[2-(3-{[3- (dimethylamino)cyclobutyl]methoxy} -1-propynyl)-4-pyrimidinyl]-5- methyl-1,4-diazepan-1-yl]-6-amino-3- pyridazinyl}phenol |
| P-252 | 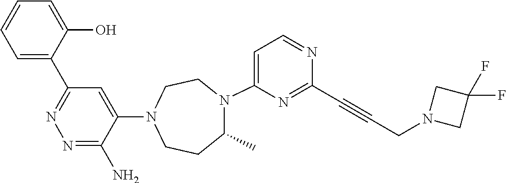 [SMILES: C[C@@H]1CCN(c2cc(-c3ccccc3O)nnc2N)CCN1c1ccnc(C#CCN2CC(F)(F)C2)n1] | o-{5-[(R)-4-{2-[3-(3,3-difluoro-1- azetidinyl)-1-propynyl]-4- pyrimidinyl}-5-methyl-1,4-diazepan- 1-yl]-6-amino-3-pyridazinyl}phenol |
| P-253 | 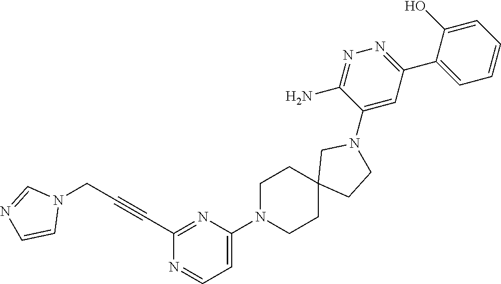 [SMILES: Nc1nnc(-c2ccccc2O)cc1N1CCC2(CCN(c3ccnc(C#CCn4ccnc4)n3)CC2)C1] | o-[6-amino-5-(8-{2-[3-(1- imidazolyl)-1-propynyl]-4- pyrimidinyl}-2,8-diaza-2- spiro[4.5]decyl)-3-pyridazinyl]phenol |
| P-254 | 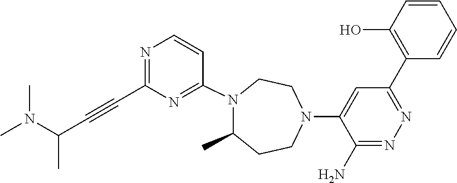 [SMILES: CC(C#Cc1nccc(N2CCN(c3cc(-c4ccccc4O)nnc3N)CC[C@H]2C)n1)N(C)C] | o-{5-[(R)-4-{2-[3-(dimethylamino)-1- butynyl]-4-pyrimidinyl}-5-methyl- 1,4-diazepan-1-yl]-6-amino-3- pyridazinyl}phenol |
| P-255 | 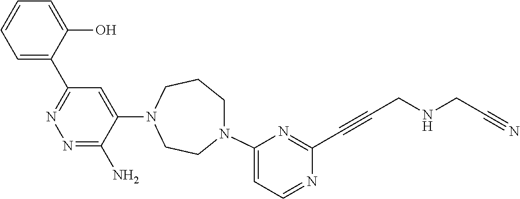 [SMILES: N#CCNCC#Cc1nccc(N2CCCN(c3cc(-c4ccccc4O)nnc3N)CC2)n1] | [3-(4-{4-[3-amino-6-(o- hydroxyphenyl)-4-pyridazinyl]-1,4- diazepan-1-yl}-2-pyrimidinyl)-2- propynylamino]acetonitrile |
| P-256 | 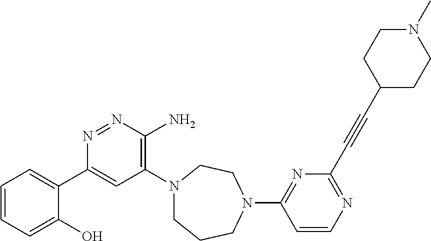 [SMILES: CN1CCC(C#Cc2nccc(N3CCCN(c4cc(-c5ccccc5O)nnc4N)CC3)n2)CC1] | o-[6-amino-5-(4-{2-[2-(1-methyl-4- piperidyl)ethynyl]-4-pyrimidinyl}- 1,4-diazepan-1-yl)-3- pyridazinyl]phenol |
| P-257 | 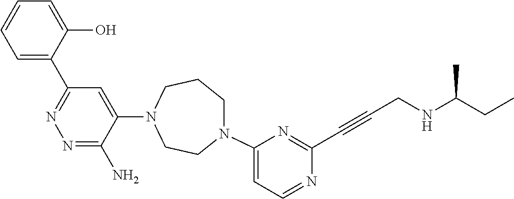 [SMILES: CC[C@H](C)NCC#Cc1nccc(N2CCCN(c3cc(-c4ccccc4O)nnc3N)CC2)n1] | o-{5-[4-(2-{3-[(S)-1- methylpropylamino]-1-propynyl}-4- pyrimidinyl)-1,4-diazepan-1-yl]-6- amino-3-pyridazinyl}phenol |
| P-258 | 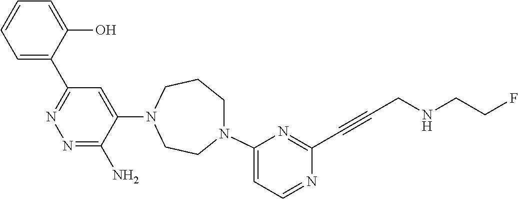 [SMILES: Nc1nnc(-c2ccccc2O)cc1N1CCCN(c2ccnc(C#CCNCCF)n2)CC1] | o-[6-amino-5-(4-{2-[3-(2- fluoroethylamino)-1-propynyl]-4- pyrimidinyl}-1,4-diazepan-1-yl)-3- pyridazinyl]phenol |
| P-259 | 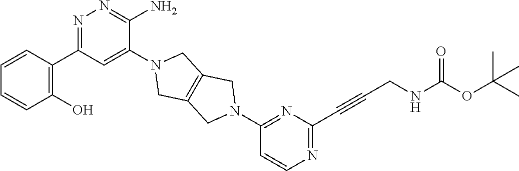 [SMILES: CC(C)(C)OC(=O)NCC#Cc1nccc(N2CC3=C(C2)CN(c2cc(-c4ccccc4O)nnc2N)C3)n1] | 3-(4-{7-[3-amino-6-(o- hydroxyphenyl)-4-pyridazinyl]-3,7- diazabicyclo[3.3.0]oct-1(5)-en-3-yl}- 2-pyrimidinyl)-2-propynylamino-tert- butylformylate |
| P-260 | 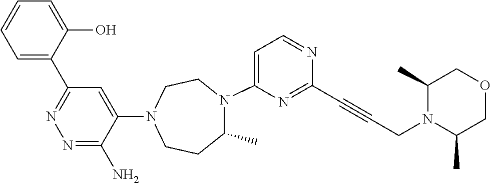 [SMILES: C[C@@H]1CCN(c2cc(-c3ccccc3O)nnc2N)CCN1c1ccnc(C#CCN2[C@H](C)COC[C@@H]2C)n1] | o-{5-[(R)-4-(2-{3-[(3R,5S)-3,5- dimethyl-4-morpholinyl]-1- propynyl}-4-pyrimidinyl)-5-methyl- 1,4-diazepan-1-yl]-6-amino-3- pyridazinyl}phenol |
| P-261 | 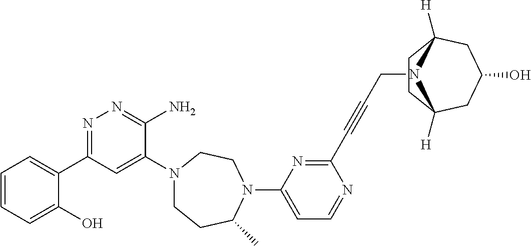 [SMILES: C[C@@H]1CCN(c2cc(-c3ccccc3O)nnc2N)CCN1c1ccnc(C#CCN2[C@@H]3CC[C@H]2C[C@@H](O)C3)n1] | 8-[3-(4-{(R)-4-[3-amino-6-(o- hydroxyphenyl)-4-pyridazinyl]-7- methyl-1,4-diazepan-1-yl}-2- pyrimidinyl)-2-propynyl]-8- azabicyclo[3.2.1]octan-3-ol |
| P-262 | 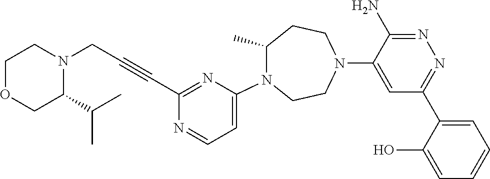 [SMILES: CC(C)[C@@H]1COCCN1CC#Cc1nccc(N2CCN(c3cc(-c4ccccc4O)nnc3N)CC[C@H]2C)n1] | o-{5-[(R)-4-(2-{3-[(R)-3-isopropyl-4- morpholinyl]-1-propynyl}-4- pyrimidinyl)-5-methyl-1,4-diazepan- 1-yl]-6-amino-3-pyridazinyl}phenol |
| P-263 | 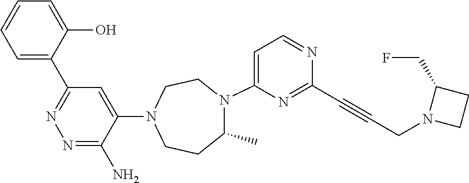 [SMILES: C[C@@H]1CCN(c2cc(-c3ccccc3O)nnc2N)CCN1c1ccnc(C#CCN2CC[C@H]2CF)n1] | o-(5-[(R)-4-(2-{3-[(S)-2- (fluoromethyl)-1-azetidinyl]-1- propynyl}-4-pyrimidinyl)-5-methyl- 1,4-diazepan-1-yl]-6-amino-3- pyridazinyl}phenol |
| P-264 | 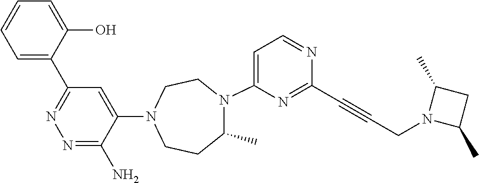 [SMILES: C[C@@H]1CCN(c2cc(-c3ccccc3O)nnc2N)CCN1c1ccnc(C#CCN2[C@H](C)C[C@H]2C)n1] | o-{5-[(R)-4-(2-{3-[(2R,4R)-2,4- dimethyl-1-azetidinyl]-1-propynyl}- 4-pyrimidinyl)-5-methyl-1,4- diazepan-1-yl]-6-amino-3- pyridazinyl}phenol |
| P-265 | 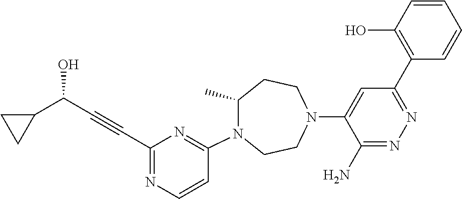 [SMILES: C[C@@H]1CCN(c2cc(-c3ccccc3O)nnc2N)CCN1c1ccnc(C#C[C@@H](O)C2CC2)n1] | o-{5-[(R)-4-{2-[(S)-3-cyclopropyl-3- hydroxy-1-propynyl]-4-pyrimidinyl}- 5-methyl-1,4-diazepan-1-yl]-6-amino- 3-pyridazinyl}phenol |
| P-266 | 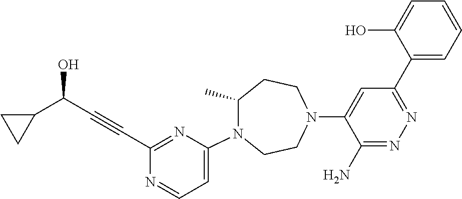 [SMILES: C[C@@H]1CCN(c2cc(-c3ccccc3O)nnc2N)CCN1c1ccnc(C#C[C@H](O)C2CC2)n1] | o-{5-[(R)-4-{2-[(R)-3-cyclopropyl-3- hydroxy-1-propynyl]-4-pyrimidinyl}- 5-methyl-1,4-diazepan-1-yl]-6-amino- 3-pyridazinyl}phenol |
| P-267 | 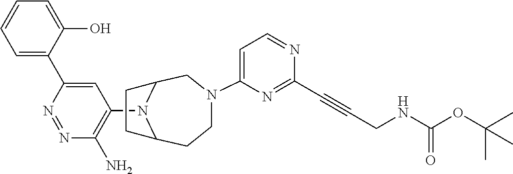 [SMILES: CC(C)(C)OC(=O)NCC#Cc1nccc(N2CCC3CCC(C2)N3c2cc(-c3ccccc3O)nnc2N)n1] | 3-(4-{9-[3-amino-6-(o- hydroxyphenyl)-4-pyridazinyl]-3,9- diazabicyclo[4.2.1]non-3-yl}-2- pyrimidinyl)-2-propynylamino-tert- butylformylate |
| P-268 | 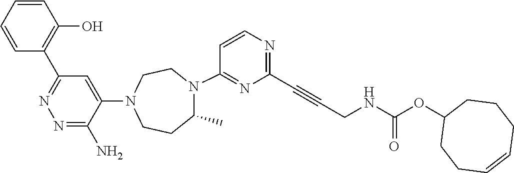 [SMILES: C[C@@H]1CCN(c2cc(-c3ccccc3O)nnc2N)CCN1c1ccnc(C#CCNC(=O)OC2CC/C=C\CCC2)n1] | 4-[(R)-4-(2-{3-[(Z)-4-cycloocten-1- yloxycarbonylamino]-1-propynyl}-4- pyrimidinyl)-5-methyl-1,4-diazepan- 1-yl]-6-(o-hydroxyphenyl)-3- pyridazinamine |
| P-269 | 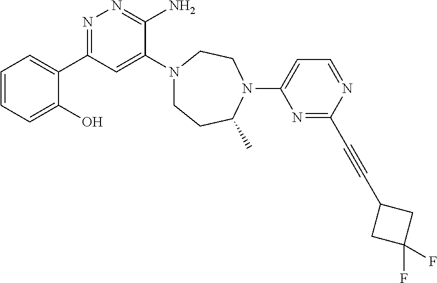 [SMILES: C[C@@H]1CCN(c2cc(-c3ccccc3O)nnc2N)CCN1c1ccnc(C#CC2CC(F)(F)C2)n1] | o-{5-[(R)-4-{2-[2-(3,3- difluorocyclobutyl)ethynyl]-4- pyrimidinyl}-5-methyl-1,4-diazepan- 1-yl]-6-amino-3-pyridazinyl}phenol |
| P-270 | 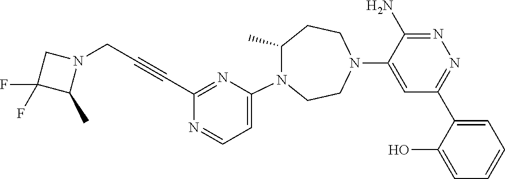 [SMILES: C[C@@H]1CCN(c2cc(-c3ccccc3O)nnc2N)CCN1c1ccnc(C#CCN2CC(F)(F)[C@@H]2C)n1] | o-{5-[(R)-4-(2-{3-[(S)-3,3-difluoro-2- methyl-1-azetidinyl]-1-propynyl}-4- pyrimidinyl)-5-methyl-1,4-diazepan- 1-yl]-6-amino-3-pyridazinyl}phenol |
| P-271 | 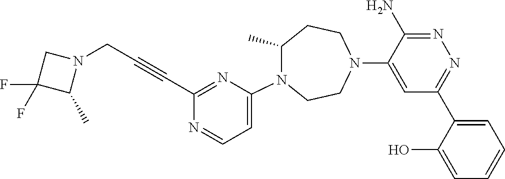 [SMILES: C[C@@H]1CCN(c2cc(-c3ccccc3O)nnc2N)CCN1c1ccnc(C#CCN2CC(F)(F)[C@H]2C)n1] | o-{5-[(R)-4-(2-{3-[(R)-3,3-difluoro- 2-methyl-1-azetidinyl]-1-propynyl}- 4-pyrimidinyl)-5-methyl-1,4- diazepan-1-yl]-6-amino-3- pyridazinyl}phenol |
| P-272 | 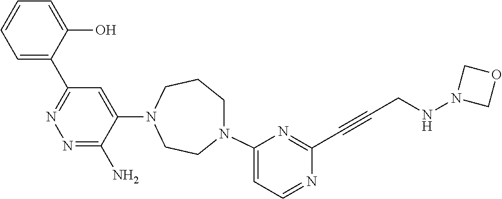 [SMILES: Nc1nnc(-c2ccccc2O)cc1N1CCCN(c2ccnc(C#CCNN3COC3)n2)CC1] | o-[6-amino-5-(4-{2-[3-(3- oxetanylamino)-1-propynyl]-4- pyrimidinyl}-1,4-diazepan-1-yl)-3- pyridazinyl]phenol |
| P-273 | 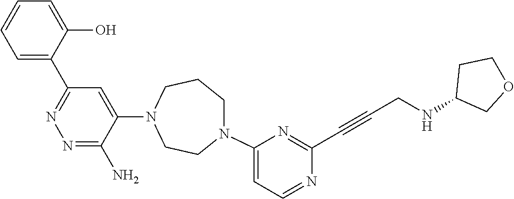 [SMILES: Nc1nnc(-c2ccccc2O)cc1N1CCCN(c2ccnc(C#CCN[C@@H]3CCOC3)n2)CC1] | o-{5-[4-(2-{3-[(R)-tetrahydro-3- furylamino]-1-propynyl}-4- pyrimidinyl)-1,4-diazepan-1-yl]-6- amino-3-pyridazinyl}phenol |
| P-274 | 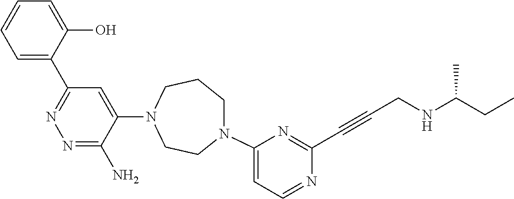 [SMILES: CC[C@@H](C)NCC#Cc1nccc(N2CCCN(c3cc(-c4ccccc4O)nnc3N)CC2)n1] | o-{5-[4-(2-{3-[(R)-1- methylpropylamino]-1-propynyl}-4- pyrimidinyl)-1,4-diazepan-1-yl]-6- amino-3-pyridazinyl}phenol |
| P-275 | 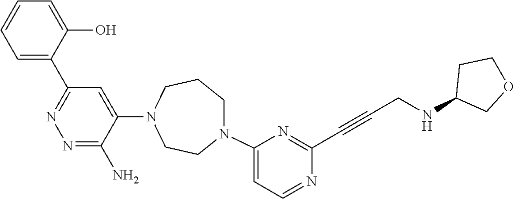 [SMILES: Nc1nnc(-c2ccccc2O)cc1N1CCCN(c2ccnc(C#CCN[C@H]3CCOC3)n2)CC1] | o-{5-14-(2-{3-[(S)-tetrahydro-3- furylamino]-1-propynyl}-4- pyrimidinyl)-1,4-diazepan-1-yl]-6- amino-3-pyridazinyl}phenol |
| P-276 | 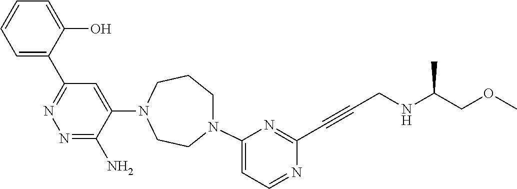 [SMILES: COC[C@H](C)NCC#Cc1nccc(N2CCCN(c3cc(-c4ccccc4O)nnc3N)CC2)n1] | o-{5-[4-(2-{3-[(S)-2-methoxy-1- methylethylamino]-1-propynyl}-4- pyrimidinyl)-1,4-diazepan-1-yl]-6- amino-3-pyridazinyl}phenol |
| P-277 | 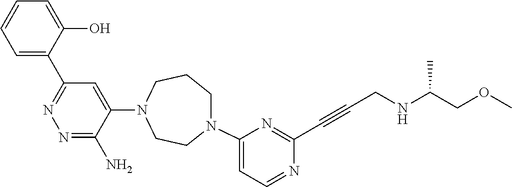 [SMILES: COC[C@@H](C)NCC#Cc1nccc(N2CCCN(c3cc(-c4ccccc4O)nnc3N)CC2)n1] | o-{5-[4-(2-{3-[(R)-2-methoxy-1- methylethylamino]-1-propynyl}-4- pyrimidinyl)-1,4-diazepan-1-yl]-6- amino-3-pyridazinyl}phenol |
| P-278 | 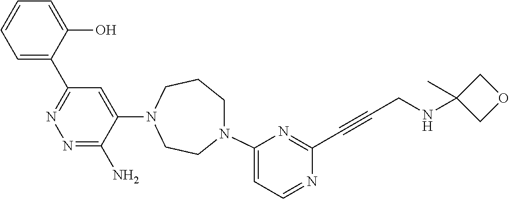 [SMILES: CC1(NCC#Cc2nccc(N3CCCN(c4cc(-c5ccccc5O)nnc4N)CC3)n2)COC1] | o-[6-amino-5-(4-{2-[3-(3-methyl-3- oxetanylamino)-1-propynyl]-4- pyrimidinyl}-1,4-diazepan-1-yl)-3- pyridazinyl]phenol |
| P-279 | 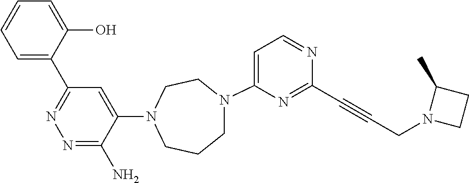 [SMILES: C[C@H]1CCN1CC#Cc1nccc(N2CCCN(c3cc(-c4ccccc4O)nnc3N)CC2)n1] | o-{5-[4-(2-{3-[(S)-2-methyl-1- azetidinyl]-1-propynyl}-4- pyrimidinyl)-1,4-diazepan-1-yl]-6- amino-3-pyridazinyl}phenol |
| P-280 | 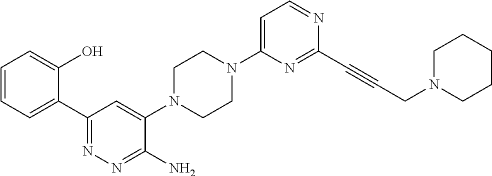 [SMILES: Nc1nnc(-c2ccccc2O)cc1N1CCN(c2ccnc(C#CCN3CCCCC3)n2)CC1] | o-(6-amino-5-{4-[2-(3-piperidino-1- propynyl)-4-pyrimidinyl]-1- piperazinyl}-3-pyridazinyl)phenol |
| P-281 | 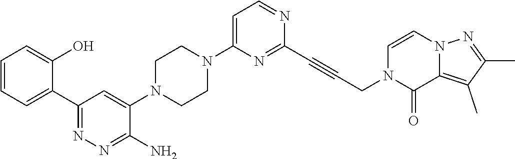 [SMILES: Cc1nn2ccn(CC#Cc3nccc(N4CCN(c5cc(-c6ccccc6O)nnc5N)CC4)n3)c(=O)c2c1C] | 5-13-(4-{4-[3-amino-6-(o- hydroxyphenyl)-4-pyridazinyl]-1- piperazinyl}-2-pyrimidinyl)-2- propynyl]-2,3-dimethyl-1,5,7a-triaza- 4(5H)-indenone |
| P-282 | 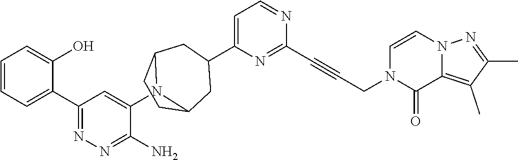 [SMILES: Cc1nn2ccn(CC#Cc3nccc(C4CC5CCC(C4)N5c4cc(-c5ccccc5O)nnc4N)n3)c(=O)c2c1C] | 5-[3-(4-{8-[3-amino-6-(o- hydroxyphenyl)-4-pyridazinyl]-3,8- diazabicyclo[3.2.1]oct-3-yl}-2- pyrimidinyl)-2-propynyl]-2,3- dimethyl-1,5,7a-triaza-4(5H)- indenone |
| P-283 | 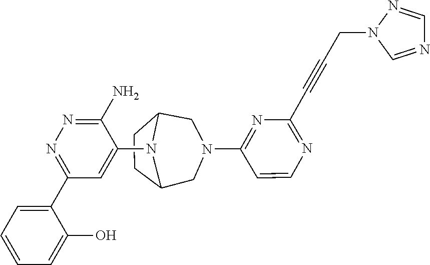 [SMILES: Nc1nnc(-c2ccccc2O)cc1N1C2CCC1CN(c1ccnc(C#CCn3cncn3)n1)C2] | o-[6-amino-5-(3-{2-[3-(1H-1,2,4- triazol-1-yl)-1-propynyl]-4- pyrimidinyl}-3,8- diazabicyclo[3.2.1]oct-8-yl)-3- pyridazinyl]phenol |
| P-284 | 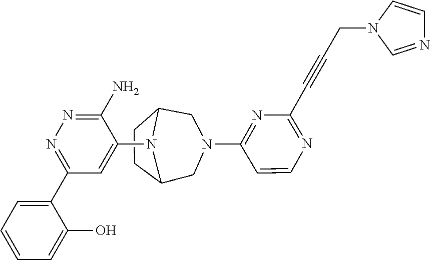 [SMILES: Nc1nnc(-c2ccccc2O)cc1N1C2CCC1CN(c1ccnc(C#CCn3ccnc3)n1)C2] | o-[6-amino-5-(3-{2-[3-(1- imidazolyl)-1-propynyl]-4- pyrimidinyl}-3,8- diazabicyclo[3.2.1]oct-8-yl)-3- pyridazinyl]phenol |
| P-285 | 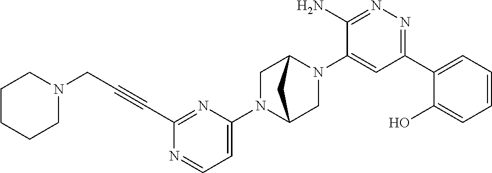 [SMILES: Nc1nnc(-c2ccccc2O)cc1N1C[C@H]2C[C@@H]1CN2c1ccnc(C#CCN2CCCCC2)n1] | o-(5-{(1R,4R)-5-[2-(3-piperidino-1- propynyl)-4-pyrimidinyl]-2,5- diazabicyclo[2.2.1]hept-2-yl}-6- amino-3-pyridazinyl)phenol |
| P-286 | 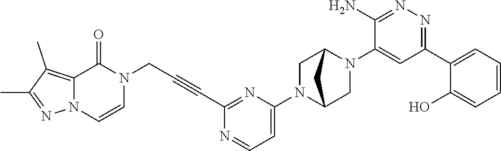 [SMILES: Cc1nn2ccn(CC#Cc3nccc(N4C[C@H]5C[C@@H]4CN5c4cc(-c5ccccc5O)nnc4N)n3)c(=O)c2c1C] | 5-[3-(4-{(1R,4R)-5-[3-amino-6-(o- hydroxyphenyl)-4-pyridazinyl]-2,5- diazabicyclo[2.2.1]hept-2-yl}-2- pyrimidinyl)-2-propynyl]-2,3- dimethyl-1,5,7a-triaza-4(5H)- indenone |
| P-287 | 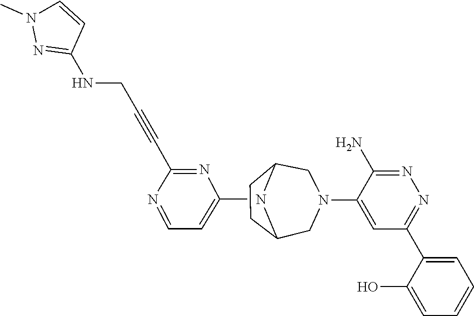 [SMILES: Cn1ccc(NCC#Cc2nccc(N3C4CCC3CN(c3cc(-c5ccccc5O)nnc3N)C4)n2)n1] | o-[6-amino-5-(8-{2-[3-(1-methyl-3- pyrazolylamino)-1-propynyl]-4- pyrimidinyl}-3,8- diazabicyclo[3.2.1]oct-3-yl)-3- pyridazinyl]phenol |
| P-288 | 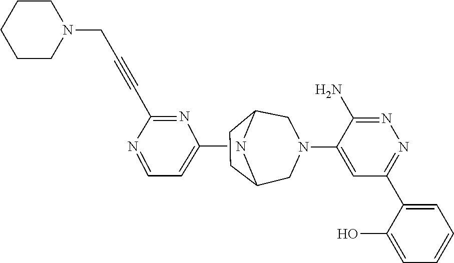 [SMILES: Nc1nnc(-c2ccccc2O)cc1N1CC2CCC(C1)N2c1ccnc(C#CCN2CCCCC2)n1] | o-(6-amino-5-{8-[2-(3-piperidino-1- propynyl)-4-pyrimidinyl]-3,8- diazabicyclo[3.2.1]oct-3-yl}-3- pyridazinyl)phenol |
| P-289 | 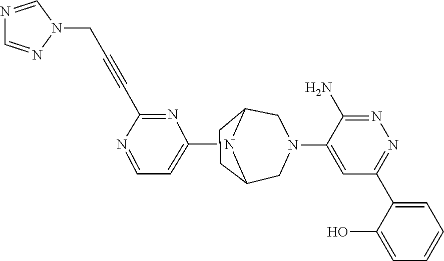 [SMILES: Nc1nnc(-c2ccccc2O)cc1N1CC2CCC(C1)N2c1ccnc(C#CCn2cncn2)n1] | o-[6-amino-5-(8-{2-[3-(1H-1,2,4- triazol-1-yl)-1-propynyl]-4- pyrimidinyl}-3,8- diazabicyclo[3.2.1]oct-3-yl)-3- pyridazinyl]phenol |
| P-290 | 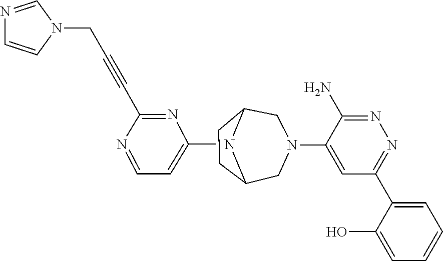 [SMILES: Nc1nnc(-c2ccccc2O)cc1N1CC2CCC(C1)N2c1ccnc(C#CCn2ccnc2)n1] | o-[6-amino-5-(8-{2-[3-(1- imidazolyl)-1-propynyl]-4- pyrimidinyl}-3,8- diazabicyclo[3.2.1]oct-3-yl)-3- pyridazinyl]phenol |
| P-291 | 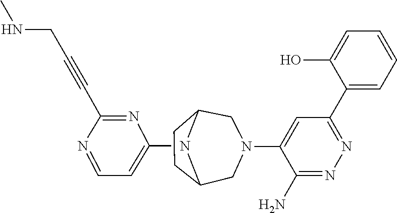 [SMILES: CNCC#Cc1nccc(N2C3CCC2CN(c2cc(-c4ccccc4O)nnc2N)C3)n1] | o-[6-amino-5-(8-{2-[3- (methylamino)-1-propynyl]-4- pyrimidinyl}-3,8- diazabicyclo[3.2.1]oct-3-yl)-3- pyridazinyl]phenol |
| P-292 | 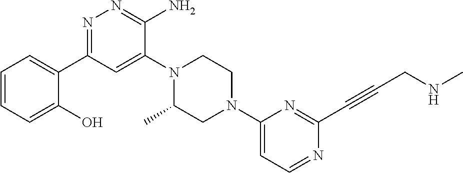 [SMILES: CNCC#Cc1nccc(N2CCN(c3cc(-c4ccccc4O)nnc3N)[C@@H](C)C2)n1] | o-{5-[(S)-2-methyl-4-{2-[3- (methylamino)-1-propynyl]-4- pyrimidinyl}-1-piperazinyl]-6-amino- 3-pyridazinyl}phenol |
| P-293 | 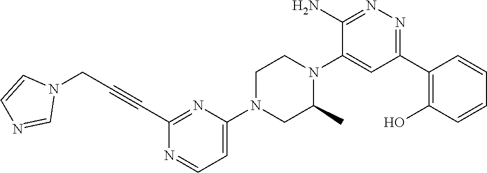 [SMILES: C[C@H]1CN(c2ccnc(C#CCn3ccnc3)n2)CCN1c1cc(-c2ccccc2O)nnc1N] | o-{5-[(S)-4-{2-[3-(1-imidazolyl)-1- propynyl]-4-pyrimidinyl}-2-methyl- 1-piperazinyl]-6-amino-3- pyridazinyl}phenol |
| P-294 | 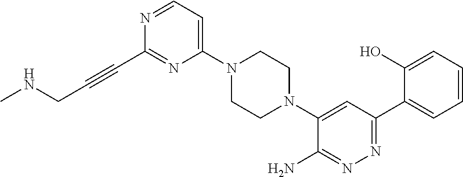 [SMILES: CNCC#Cc1nccc(N2CCN(c3cc(-c4ccccc4O)nnc3N)CC2)n1] | o-[6-amino-5-(4-{2-13- (methylamino)-1-propynyl]-4- pyrimidinyl}-1-piperazinyl)-3- pyridazinyl]phenol |
| P-295 | 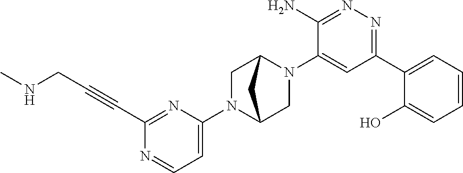 [SMILES: CNCC#Cc1nccc(N2C[C@H]3C[C@@H]2CN3c2cc(-c3ccccc3O)nnc2N)n1] | o-{5-[(1R,4R)-5-{2-[3- (methylamino)-1-propynyl]-4- pyrimidinyl}-2,5- diazabicyclo[2.2.1]hept-2-yl]-6- amino-3-pyridazinyl}phenol |
| P-296 | 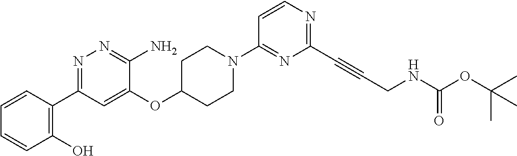 [SMILES: CC(C)(C)OC(=O)NCC#Cc1nccc(N2CCC(Oc3cc(-c4ccccc4O)nnc3N)CC2)n1] | 4-(1-{2-[3-(tert- butoxycarbonylamino)-1-propynyl]- 4-pyrimidinyl}-4-piperidyloxy)-6-(o- hydroxyphenyl)-3-pyridazinamine |
| P-297 | 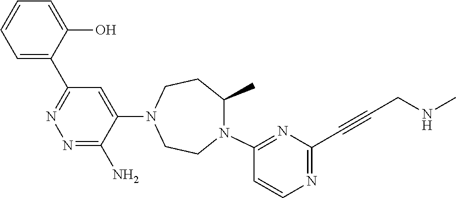 [SMILES: CNCC#Cc1nccc(N2CCN(c3cc(-c4ccccc4O)nnc3N)CC[C@H]2C)n1] | o-{5-[(R)-5-methyl-4-{2-[3- (methylamino)-1-propynyl]-4- pyrimidinyl}-1,4-diazepan-1-yl]-6- amino-3-pyridazinyl}phenol |
| P-298 | 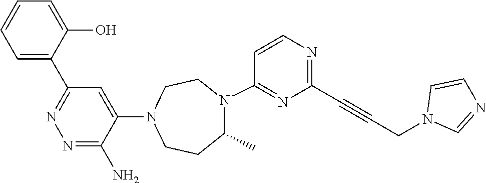 [SMILES: C[C@@H]1CCN(c2cc(-c3ccccc3O)nnc2N)CCN1c1ccnc(C#CCn2ccnc2)n1] | o-{5-[(R)-4-{2-[3-(1-imidazolyl)-1- propynyl]-4-pyrimidinyl}-5-methyl- 1,4-diazepan-1-yl]-6-amino-3- pyridazinyl}phenol |
| P-299 | 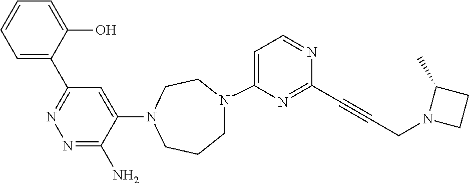 [SMILES: C[C@@H]1CCN1CC#Cc1nccc(N2CCCN(c3cc(-c4ccccc4O)nnc3N)CC2)n1] | o-{5-[4-(2-{3-[(R)-2-methyl-1- azetidinyl]-1-propynyl}-4- pyrimidinyl)-1,4-diazepan-1-yl]-6- amino-3-pyridazinyl}phenol |
| P-300 | 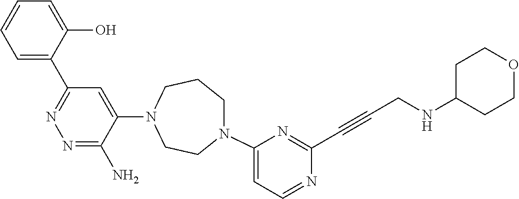 [SMILES: Nc1nnc(-c2ccccc2O)cc1N1CCCN(c2ccnc(C#CCNC3CCOCC3)n2)CC1] | o-[6-amino-5-(4-{2-[3-(tetrahydro- 2H-pyran-4-ylamino)-1-propynyl]-4- pyrimidinyl}-1,4-diazepan-1-yl)-3- pyridazinyl]phenol |
| P-301 | 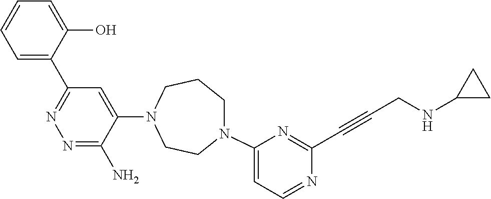 [SMILES: Nc1nnc(-c2ccccc2O)cc1N1CCCN(c2ccnc(C#CCNC3CC3)n2)CC1] | o-[6-amino-5-(4-{2-[3- (cyclopropylamino)-1-propynyl]-4- pyrimidinyl}-1,4-diazepan-1-yl)-3- pyridazinyl]phenol |
| P-302 | 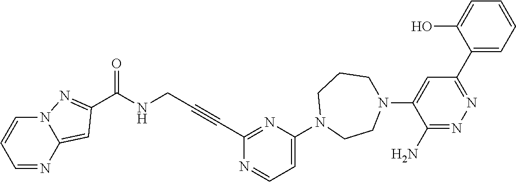 [SMILES: Nc1nnc(-c2ccccc2O)cc1N1CCCN(c2ccnc(C#CCNC(=O)c3cc4ncccn4n3)n2)CC1] | N-[3-(4-{4-[3-amino-6-(o- hydroxyphenyl)-4-pyridazinyl]-1- azepanyl}-2-pyrimidinyl)-2- propynyl]-1,4,7a-triaza-2- indenecarboxamide |
| P-303 | 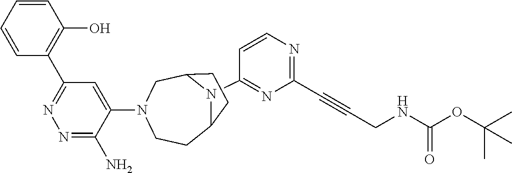 [SMILES: CC(C)(C)OC(=O)NCC#Cc1nccc(N2C3CCC2CN(c2cc(-c4ccccc4O)nnc2N)CC3)n1] | 4-(9-{2-[3-(tert- butoxycarbonylamino)-1-propynyl]- 4-pyrimidinyl}-3,9- diazabicyclo[4.2.1]non-3-yl)-6-(o- hydroxyphenyl)-3-pyridazinamine |
| P-304 | 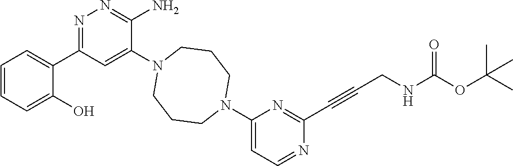 [SMILES: CC(C)(C)OC(=O)NCC#Cc1nccc(N2CCCN(c3cc(-c4ccccc4O)nnc3N)CCC2)n1] | 4-(5-{2-[3-(tert- butoxycarbonylamino)-1-propynyl]- 4-pyrimidinyl}-1,5-diazocan-1-yl)-6- (o-hydroxyphenyl)-3-pyridazinamine |
| P-305 | 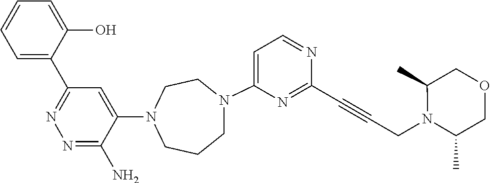 [SMILES: C[C@H]1COC[C@H](C)N1CC#Cc1nccc(N2CCCN(c3cc(-c4ccccc4O)nnc3N)CC2)n1] | o-{5-[4-(2-{3-[(3S,5S)-3,5-dimethyl- 4-morpholinyl]-1-propynyl}-4- pyrimidinyl)-1,4-diazepan-1-yl]-6- amino-3-pyridazinyl}phenol |
| P-306 | 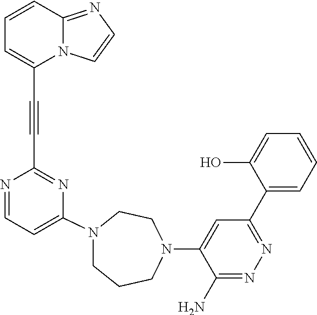 [SMILES: Nc1nnc(-c2ccccc2O)cc1N1CCCN(c2ccnc(C#Cc3cccc4nccn34)n2)CC1] | o-[5-(4-{2-[2-(1,3a-diaza-4- indenyl)ethynyl]-4-pyrimidinyl}-1,4- diazepan-1-yl)-6-amino-3- pyridazinyl]phenol |
| P-307 | 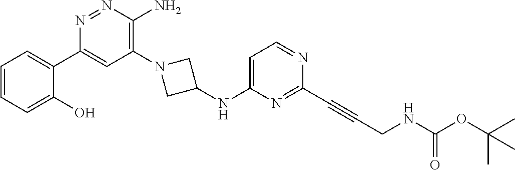 [SMILES: CC(C)(C)OC(=O)NCC#Cc1nccc(NC2CN(c3cc(-c4ccccc4O)nnc3N)C2)n1] | 4-(3-{2-[3-(tert- butoxycarbonylamino)-1-propynyl]- 4-pyrimidinylamino}-1-azetidinyl)-6- (o-hydroxyphenyl)-3-pyridazinamine |
| P-308 | 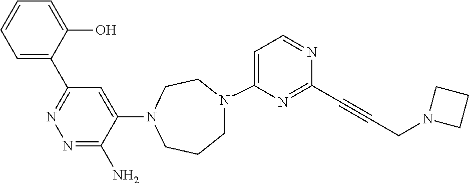 [SMILES: Nc1nnc(-c2ccccc2O)cc1N1CCCN(c2ccnc(C#CCN3CCC3)n2)CC1] | o-[6-amino-5-(4-{2-[3-(1-azetidinyl)- 1-propynyl]-4-pyrimidinyl}-1,4- diazepan-1-yl)-3-pyridazinyl]phenol |
| P-309 | 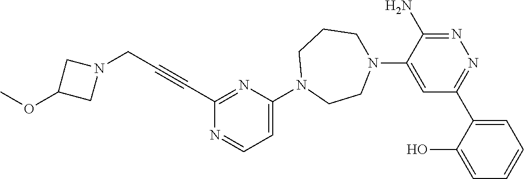 [SMILES: COC1CN(CC#Cc2nccc(N3CCCN(c4cc(-c5ccccc5O)nnc4N)CC3)n2)C1] | o-[6-amino-5-(4-{2-[3-(3-methoxy-1- azetidinyl)-1-propynyl]-4- pyrimidinyl}-1,4-diazepan-1-yl)-3- pyridazinyl]phenol |
| P-310 | 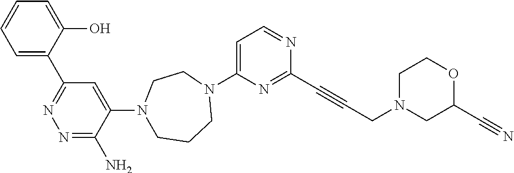 [SMILES: N#CC1CN(CC#Cc2nccc(N3CCCN(c4cc(-c5ccccc5O)nnc4N)CC3)n2)CCO1] | 4-[3-(4-{4-[3-amino-6-(o- hydroxyphenyl)-4-pyridazinyl]-1,4- diazepan-1-yl}-2-pyrimidinyl)-2- propynyl]-2-morpholinecarbonitrile |
| P-311 | 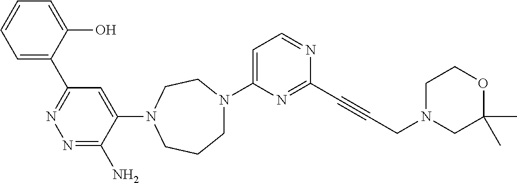 | o-[6-amino-5-(4-{2-[3-(2,2-dimethyl- 4-morpholinyl)-1-propynyl]-4- pyrimidinyl}-1,4-diazepan-1-yl)-3- pyridazinyl]phenol |
| P-312 | 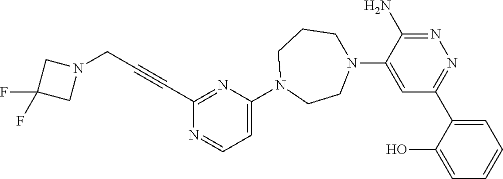 [SMILES: Nc1nnc(-c2ccccc2O)cc1N1CCCN(c2ccnc(C#CCN3CC(F)(F)C3)n2)CC1] | o-[6-amino-5-(4-{2-[3-(3,3-difluoro- 1-azetidinyl)-1-propynyl]-4- pyrimidinyl}-1,4-diazepan-1-yl)-3- pyridazinyl]phenol |
| P-313 | 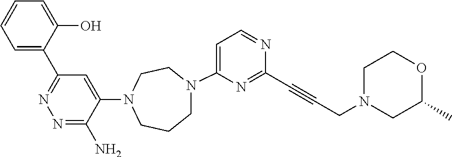 [SMILES: C[C@@H]1CN(CC#Cc2nccc(N3CCCN(c4cc(-c5ccccc5O)nnc4N)CC3)n2)CCO1] | o-{5-[4-(2-{3-[(R)-2-methyl-4- morpholinyl]-1-propynyl}-4- pyrimidinyl)-1,4-diazepan-1-yl]-6- amino-3-pyridazinyl}phenol |
| P-314 | 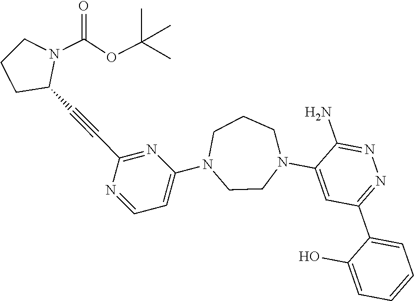 [SMILES: CC(C)(C)OC(=O)N1CCC[C@H]1C#Cc1nccc(N2CCCN(c3cc(-c4ccccc4O)nnc3N)CC2)n1] | tert-butyl (S)-2-[2-(4-{4-[3-amino-6- (o-hydroxyphenyl)-4-pyridazinyl]- 1,4-diazepan-1-yl}-2- pyrimidinyl)ethynyl]-1- pyrrolidinecarboxylate |
| P-315 | 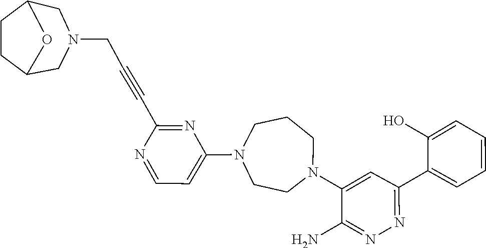 [SMILES: Nc1nnc(-c2ccccc2O)cc1N1CCCN(c2ccnc(C#CCN3CC4CCC(C3)O4)n2)CC1] | o-{6-amino-5-[4-(2-{3-(8-oxa-3- azabicyclo[3.2.1]oct-3-yl)-1- propynyl}-4-pyrimidinyl)-1,4- diazepan-1-yl]-3-pyridazinyl}phenol |
| P-316 | 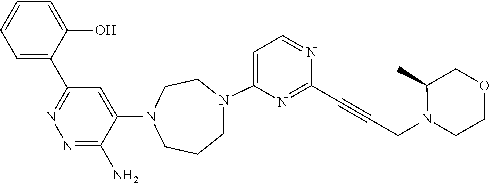 [SMILES: C[C@H]1COCCN1CC#Cc1nccc(N2CCCN(c3cc(-c4ccccc4O)nnc3N)CC2)n1] | o-{5-[4-(2-{3-[(S)-3-methyl-4- morpholinyl]-1-propynyl}-4- pyrimidinyl)-1,4-diazepan-1-yl]-6- amino-3-pyridazinyl}phenol |
| P-317 | 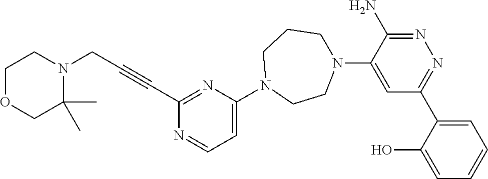 | o-[6-amino-5-(4-{2-[3-(3,3-dimethyl- 4-morpholinyl)-1-propynyl]-4- pyrimidinyl}-1,4-diazepan-1-yl)-3- pyridazinyl]phenol |
| P-318 | 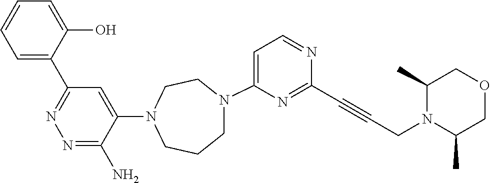 [SMILES: C[C@@H]1COC[C@H](C)N1CC#Cc1nccc(N2CCCN(c3cc(-c4ccccc4O)nnc3N)CC2)n1] | o-{5-[4-(2-{3-[(3R,5S)-3,5-dimethyl- 4-morpholinyl]-1-propynyl}-4- pyrimidinyl)-1,4-diazepan-1-yl]-6- amino-3-pyridazinyl}phenol |
| P-319 | 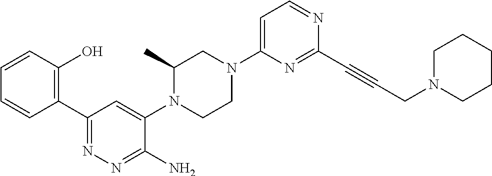 [SMILES: C[C@H]1CN(c2ccnc(C#CCN3CCCCC3)n2)CCN1c1cc(-c2ccccc2O)nnc1N] | o-(5-{(S)-2-methyl-4-[2-(3- piperidino-1-propynyl)-4- pyrimidinyl]-1-piperazinyl}-6-amino- 3-pyridazinyl)phenol |
| P-320 | 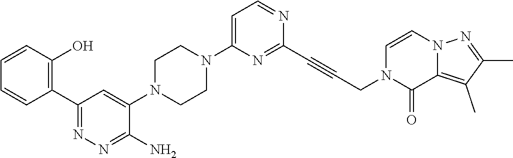 [SMILES: Cc1nn2ccn(CC#Cc3nccc(N4CCN(c5cc(-c6ccccc6O)nnc5N)CC4)n3)c(=O)c2c1C] | 5-[3-(4-{(S)-4-[3-amino-6-(o- hydroxyphenyl)-4-pyridazinyl]-3- methyl-1-piperazinyl}-2- pyrimidinyl)-2-propynyl]-2,3- dimethyl-1,5,7a-triaza-4(5H)- indenone |
| P-321 | 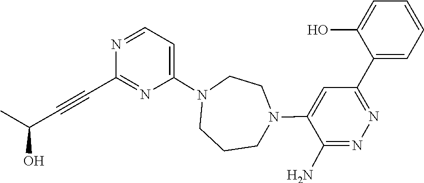 [SMILES: C[C@H](O)C#Cc1nccc(N2CCCN(c3cc(-c4ccccc4O)nnc3N)CC2)n1] | o-[5-(4-{2-[(S)-3-hydroxy-1- butynyl]-4-pyrimidinyl}-1,4- diazepan-1-yl)-6-amino-3- pyridazinyl]phenol |
| P-322 | 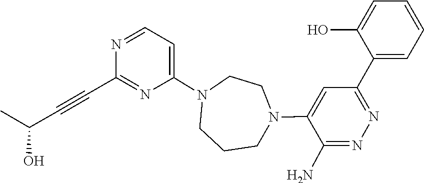 [SMILES: C[C@@H](O)C#Cc1nccc(N2CCCN(c3cc(-c4ccccc4O)nnc3N)CC2)n1] | o-[5-(4-{2-[(R)-3-hydroxy-1- butynyl]-4-pyrimidinyl}-1,4- diazepan-1-yl)-6-amino-3- pyridazinyl]phenol |
| P-323 | 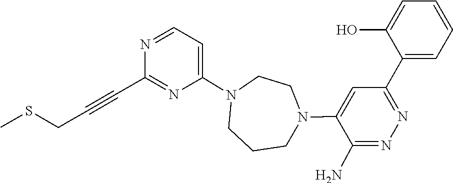 [SMILES: CSCC#Cc1nccc(N2CCCN(c3cc(-c4ccccc4O)nnc3N)CC2)n1] | o-[6-amino-5-(4-(2-[3-(methylthio)- 1-propynyl]-4-pyrimidinyl}-1,4- diazepan-1-yl)-3-pyridazinyl]phenol |
| P-324 | 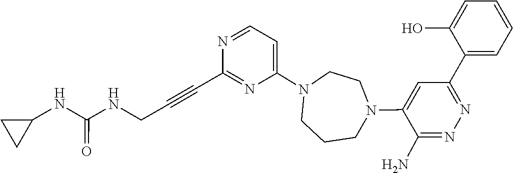 [SMILES: Nc1nnc(-c2ccccc2O)cc1N1CCCN(c2ccnc(C#CCNC(=O)NC3CC3)n2)CC1] | o-[6-amino-5-(4-{2-[3-(3- cyclopropylureido)-1-propynyl]-4- pyrimidinyl}-1,4-diazepan-1-yl)-3- pyridazinyl]phenol |
| P-325 | 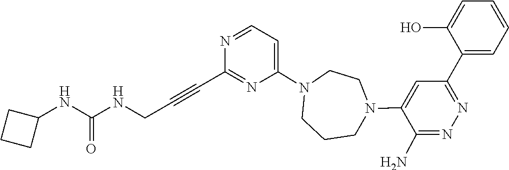 [SMILES: Nc1nnc(-c2ccccc2O)cc1N1CCCN(c2ccnc(C#CCNC(=O)NC3CCC3)n2)CC1] | o-[6-amino-5-(4-{2-[3-(3- cyclobutylureido)-1-propynyl]-4- pyrimidinyl}-1,4-diazepan-1-yl)-3- pyridazinyl]phenol |
| P-326 | 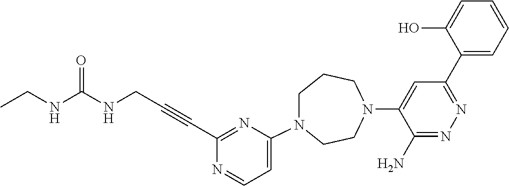 [SMILES: CCNC(=O)NCC#Cc1nccc(N2CCCN(c3cc(-c4ccccc4O)nnc3N)CC2)n1] | o-[6-amino-5-(4-(2-[3-(3- ethylureido)-1-propynyl]-4- pyrimidinyl}-1,4-diazepan-1-yl)-3- pyridazinyl]phenol |
| P-327 | 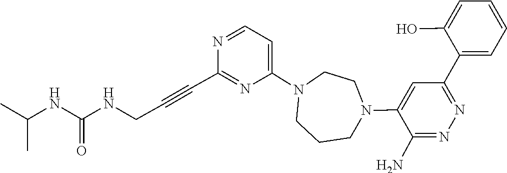 [SMILES: CC(C)NC(=O)NCC#Cc1nccc(N2CCCN(c3cc(-c4ccccc4O)nnc3N)CC2)n1] | o-[6-amino-5-(4-{2-[3-(3- isopropylureido)-1-propynyl]-4- pyrimidinyl}-1,4-diazepan-1-yl)-3- pyridazinyl]phenol |
| P-328 | 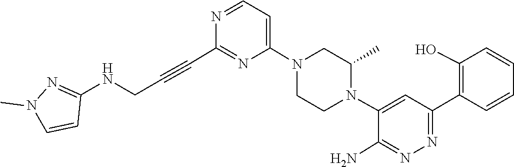 [SMILES: C[C@H]1CN(c2ccnc(C#CCNc3ccn(C)n3)n2)CCN1c1cc(-c2ccccc2O)nnc1N] | o-{5-[(S)-2-methyl-4-{2-[3-(1- methyl-3-pyrazolylamino)-1- propynyl]-4-pyrimidinyl}-1- piperazinyl]-6-amino-3- pyridazinyl}phenol |
| P-329 | 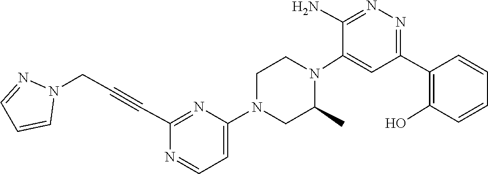 [SMILES: C[C@H]1CN(c2ccnc(C#CCn3cccn3)n2)CCN1c1cc(-c2ccccc2O)nnc1N] | o-{5-[(S)-2-methyl-4-{2-[3-(1- pyrazolyl)-1-propynyl]-4- pyrimidinyl}-1-piperazinyl]-6-amino- 3-pyridazinyl}phenol |
| P-330 | 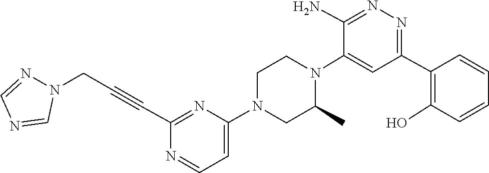 [SMILES: C[C@H]1CN(c2ccnc(C#CCn3cncn3)n2)CCN1c1cc(-c2ccccc2O)nnc1N] | o-{5-[(S)-2-methyl-4-{2-[3-(1H- 1,2,4-triazol-1-yl)-1-propynyl]-4- pyrimidinyl}-1-piperazinyl}-6-amino- 3-pyridazinyl}phenol |
| P-331 | 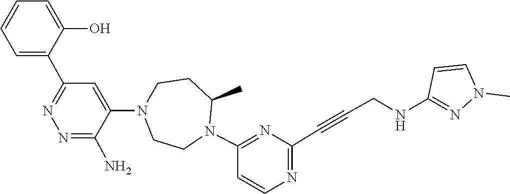 [SMILES: C[C@@H]1CCN(c2cc(-c3ccccc3O)nnc2N)CCN1c1ccnc(C#CCNc2ccn(C)n2)n1] | o-{5-[(R)-5-methyl-4-{2-[3-(1- methyl-3-pyrazolylamino)-1- propynyl]-4-pyrimidinyl}-1,4- diazepan-1-yl]-6-amino-3- pyridazinyl}phenol |
| P-332 | 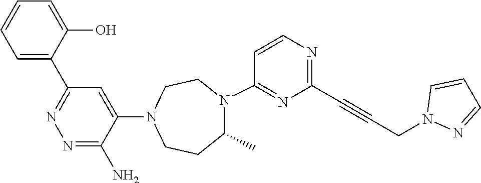 [SMILES: C[C@@H]1CCN(c2cc(-c3ccccc3O)nnc2N)CCN1c1ccnc(C#CCn2cccn2)n1] | o-{5-[(R)-5-methyl-4-{2-[3-(1- pyrazolyl)-1-propynyl]-4- pyrimidinyl}-1,4-diazepan-1-yl]-6- amino-3-pyridazinyl}phenol |
| P-333 | 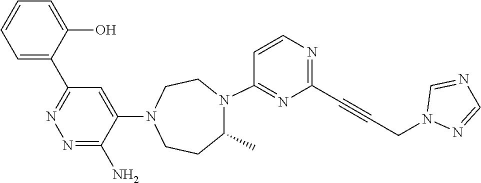 [SMILES: C[C@@H]1CCN(c2cc(-c3ccccc3O)nnc2N)CCN1c1ccnc(C#CCn2cncn2)n1] | o-{5-[(R)-5-methyl-4-{2-[3-(1H- 1,2,4-triazol-1-yl)-1-propynyl]-4- pyrimidinyl}-1,4-diazepan-1-yl]-6- amino-3-pyridazinyl}phenol |
| P-334 | 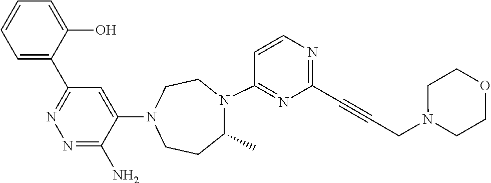 [SMILES: C[C@@H]1CCN(c2cc(-c3ccccc3O)nnc2N)CCN1c1ccnc(C#CCN2CCOCC2)n1] | o-(5-{(R)-5-methyl-4-[2-(3- morpholino-1-propynyl)-4- pyrimidinyl]-1,4-diazepan-1-yl}-6- amino-3-pyridazinyl)phenol |
| P-335 | 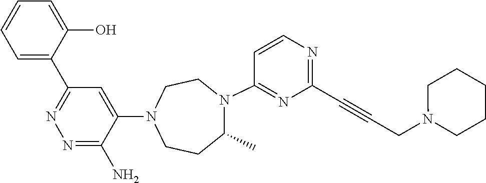 [SMILES: C[C@@H]1CCN(c2cc(-c3ccccc3O)nnc2N)CCN1c1ccnc(C#CCN2CCCCC2)n1] | o-(5-{(R)-5-methyl-4-[2-(3- piperidino-1-propynyl)-4- pyrimidinyl]-1,4-diazepan-1-yl}-6- amino-3-pyridazinyl)phenol |
| P-336 | 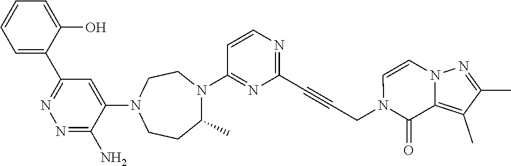 [SMILES: Cc1nn2ccn(CC#Cc3nccc(N4CCN(c5cc(-c6ccccc6O)nnc5N)CC[C@H]4C)n3)c(=O)c2c1C] | 5-[3-(4-{(R)-4-[3-amino-6-(o- hydroxyphenyl)-4-pyridazinyl]-7- methyl-1,4-diazepan-1-yl}-2- pyrimidinyl)-2-propynyl]-2,3- dimethyl-1,5,7a-triaza-4(5H)- indenone |
| P-337 | 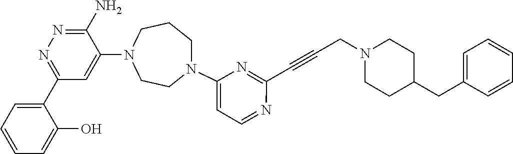 [SMILES: Nc1nnc(-c2ccccc2O)cc1N1CCCN(c2ccnc(C#CCN3CCC(Cc4ccccc4)CC3)n2)CC1] | o-[6-amino-5-(4-{2-[3-(4-benzyl-1- piperidyl)-1-propynyl]-4- pyrimidinyl}-1,4-diazepan-1-yl)-3- pyridazinyl]phenol |
| P-338 | 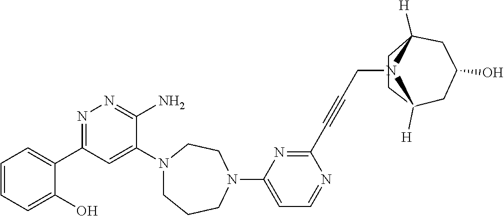 | 8-[3-(4-{4-[3-amino-6-(o- hydroxyphenyl)-4-pyridazinyl]-1,4- diazepan-1-yl}-2-pyrimidinyl)-2- propynyl]-8-azabicyclo[3.2.1]octan- 3-ol |
| P-339 | 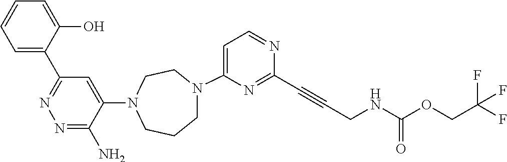 [SMILES: Nc1nnc(-c2ccccc2O)cc1N1CCCN(c2ccnc(C#CCNC(=O)OCC(F)(F)F)n2)CC1] | 6-(o-hydroxyphenyl)-4-(4-{2-[3- (2,2,2- trifluoroethoxycarbonylamino)-1- propynyl]-4-pyrimidinyl}-1,4- diazepan-1-yl)-3-pyridazinamine |
| P-340 | 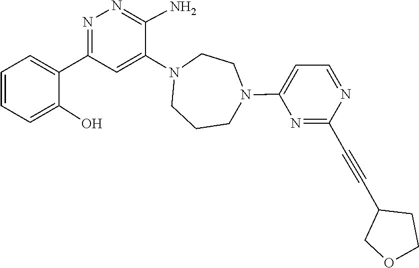 [SMILES: Nc1nnc(-c2ccccc2O)cc1N1CCCN(c2ccnc(C#CC3CCOC3)n2)CC1] | o-[6-amino-5-(4-{2-[2-(tetrahydro-3- furyl)ethynyl]-4-pyrimidinyl}-1,4- diazepan-1-yl)-3-pyridazinyl]phenol |
| P-341 | 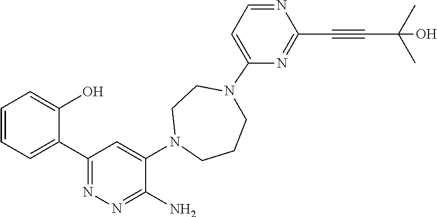 [SMILES: CC(C)(O)C#Cc1nccc(N2CCCN(c3cc(-c4ccccc4O)nnc3N)CC2)n1] | 4-(4-{4-[3-amino-6-(o- hydroxyphenyl)-4-pyridazinyl]-1,4- diazepan-1-yl}-2-pyrimidinyl)-2- methyl-3-butyn-2-ol |
| P-342 | 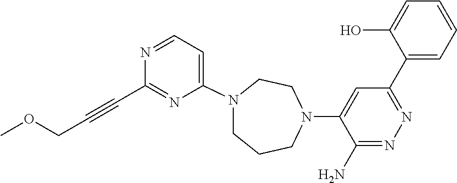 [SMILES: COCC#Cc1nccc(N2CCCN(c3cc(-c4ccccc4O)nnc3N)CC2)n1] | o-(6-amino-5-{4-[2-(3-methoxy-1- propynyl)-4-pyrimidinyl]-1,4- diazepan-1-yl}-3-pyridazinyl)phenol |
| P-343 | 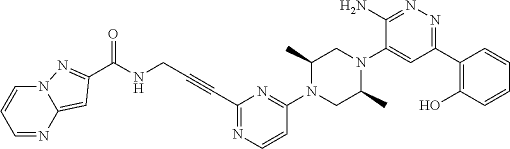 [SMILES: C[C@H]1CN(c2cc(-c3ccccc3O)nnc2N)[C@@H](C)CN1c1ccnc(C#CCNC(=O)c2cc3ncccn3n2)n1] | N-[3-(4-{(2S,5S)-4-[3-amino-6-(o- hydroxyphenyl)-4-pyridazinyl]-2,5- dimethyl-1-piperazinyl}-2- pyrimidinyl)-2-propynyl]-1,4,7a- triaza-2-indenecarboxamide |
| P-344 | 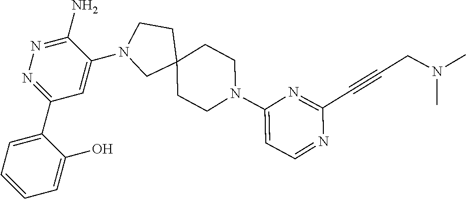 [SMILES: CN(C)CC#Cc1nccc(N2CCC3(CC2)CCN(c2cc(-c4ccccc4O)nnc2N)C3)n1] | o-[6-amino-5-(8-{2-[3- (dimethylamino)-1-propynyl]-4- pyrimidinyl}-2,8-diaza-2- spiro[4.5]decyl)-3-pyridazinyl]phenol |
| P-345 | 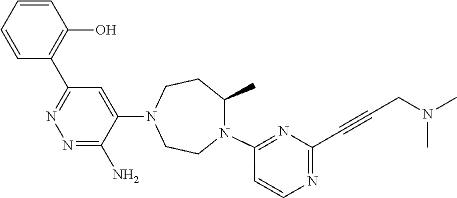 [SMILES: C[C@@H]1CCN(c2cc(-c3ccccc3O)nnc2N)CCN1c1ccnc(C#CCN(C)C)n1] | o-{5-[(R)-4-{2-[3-(dimethylamino)-1- propynyl]-4-pyrimidinyl}-5-methyl- 1,4-diazepan-1-yl]-6-amino-3- pyridazinyl}phenol |
| P-346 | 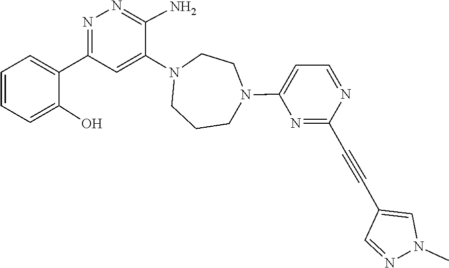 [SMILES: Cn1cc(C#Cc2nccc(N3CCCN(c4cc(-c5ccccc5O)nnc4N)CC3)n2)cn1] | o-[6-amino-5-(4-{2-[2-(1-methyl-4- pyrazolyl)ethynyl]-4-pyrimidinyl}- 1,4-diazepan-1-yl)-3- pyridazinyl]phenol |
| P-347 | 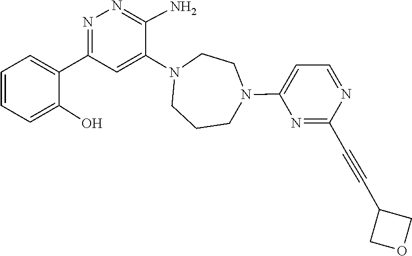 [SMILES: Nc1nnc(-c2ccccc2O)cc1N1CCCN(c2ccnc(C#CC3COC3)n2)CC1] | o-[6-amino-5-(4-{2-[2-(3- oxetanyl)ethynyl]-4-pyrimidinyl}- 1,4-diazepan-1-yl)-3- pyridazinyl]phenol |
| P-348 | 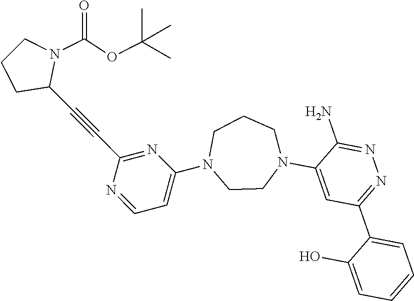 [SMILES: CC(C)(C)OC(=O)N1CCCC1C#Cc1nccc(N2CCCN(c3cc(-c4ccccc4O)nnc3N)CC2)n1] | tert-butyl 2-[2-(4-{4-[3-amino-6-(o- hydroxyphenyl)-4-pyridazinyl]-1,4- diazepan-1-yl}-2- pyrimidinyl)ethynyl]-1- pyrrolidinecarboxylate |
| P-349 | 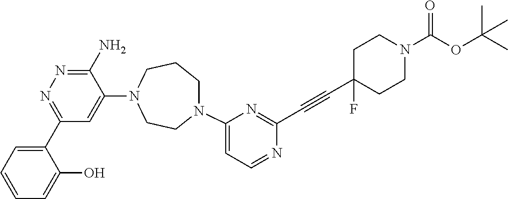 [SMILES: CC(C)(C)OC(=O)N1CCC(F)(C#Cc2nccc(N3CCCN(c4cc(-c5ccccc5O)nnc4N)CC3)n2)CC1] | tert-butyl 4-[2-(4-{4-[3-amino-6-(o- hydroxyphenyl)-4-pyridazinyl]-1,4- diazepan-1-yl}-2- pyrimidinyl)ethynyl]-4-fluoro-1- piperidinecarboxylate |
| P-350 | 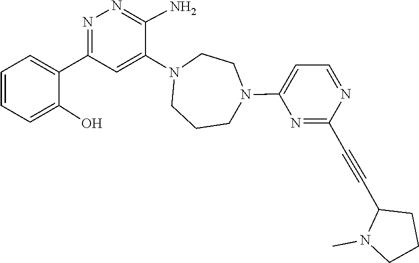 [SMILES: CN1CCCC1C#Cc1nccc(N2CCCN(c3cc(-c4ccccc4O)nnc3N)CC2)n1] | o-[6-amino-5-(4-{2-[2-(1-methyl-2- pyrrolidinyl)ethynyl]-4-pyrimidinyl}- 1,4-diazepan-1-yl)-3- pyridazinyl]phenol |
| P-351 | 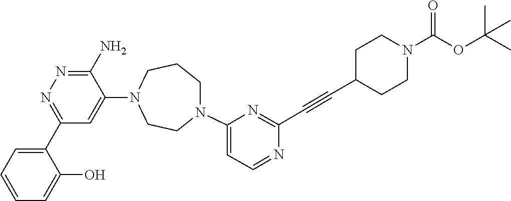 [SMILES: CC(C)(C)OC(=O)N1CCC(C#Cc2nccc(N3CCCN(c4cc(-c5ccccc5O)nnc4N)CC3)n2)CC1] | tert-butyl 4-[2-(4-{4-[3-amino-6-(o- hydroxyphenyl)-4-pyridazinyl]-1,4- diazepan-1-yl}-2- pyrimidinyl)ethynyl]-1- piperidinecarboxylate |
| P-352 | 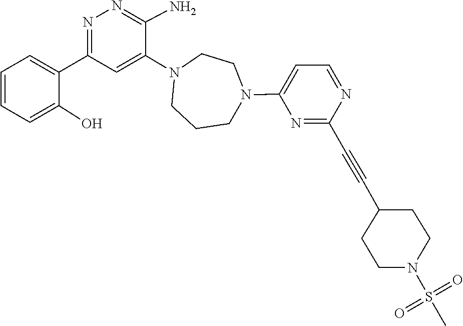 [SMILES: CS(=O)(=O)N1CCC(C#Cc2nccc(N3CCCN(c4cc(-c5ccccc5O)nnc4N)CC3)n2)CC1] | o-[6-amino-5-(4-{2-[2-(1-mesyl-4- piperidyl)ethynyl]-4-pyrimidinyl}- 1,4-diazepan-1-yl)-3- pyridazinyl]phenol |
| P-353 | 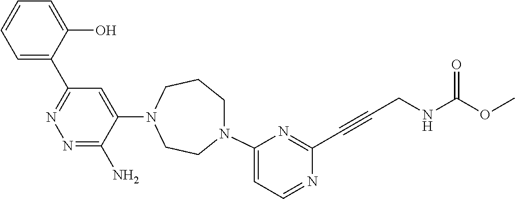 [SMILES: COC(=O)NCC#Cc1nccc(N2CCCN(c3cc(-c4ccccc4O)nnc3N)CC2)n1] | 3-(4-{4-[3-amino-6-(o- hydroxyphenyl)-4-pyridazinyl]-1,4- diazepan-1-yl}-2-pyrimidinyl)-2- propynylaminomethylformylate |
| P-354 | 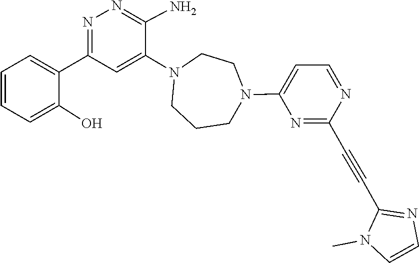 [SMILES: Cn1ccnc1C#Cc1nccc(N2CCCN(c3cc(-c4ccccc4O)nnc3N)CC2)n1] | o-[6-amino-5-(4-{2-[2-(1-methyl-2- imidazolyl)ethynyl]-4-pyrimidinyl}- 1,4-diazepan-1-yl)-3- pyridazinyl]phenol |
| P-355 | 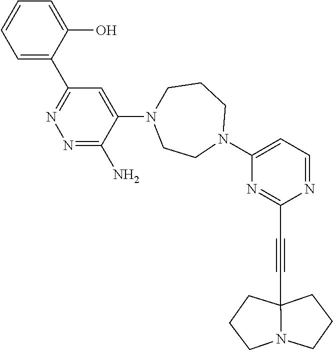 [SMILES: Nc1nnc(-c2ccccc2O)cc1N1CCCN(c2ccnc(C#CC34CCCN3CCC4)n2)CC1] | o-[6-amino-5-(4-{2-[2-(hexahydro- 1H-pyrrolizin-7a-yl)ethynyl]-4- pyrimidinyl}-1,4-diazepan-1-yl)-3- pyridazinyl]phenol |
| P-356 | 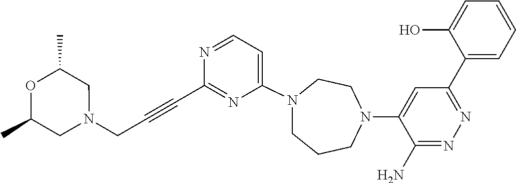 [SMILES: C[C@@H]1CN(CC#Cc2nccc(N3CCCN(c4cc(-c5ccccc5O)nnc4N)CC3)n2)C[C@@H](C)O1] | o-(5-[4-(2-(3-[(2R,6R)-2,6-dimethyl- 4-morpholinyl]-1-propynyl}-4- pyrimidinyl)-1,4-diazepan-1-yl]-6- amino-3-pyridazinyl}phenol |
| P-357 | 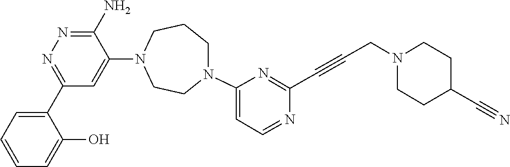 [SMILES: N#CC1CCN(CC#Cc2nccc(N3CCCN(c4cc(-c5ccccc5O)nnc4N)CC3)n2)CC1] | 1-[3-(4-{4-[3-amino-6-(o- hydroxyphenyl)-4-pyridazinyl]-1,4- diazepan-1-yl}-2-pyrimidinyl)-2- propynyl]-4-piperidinecarbonitrile |
| P-358 | 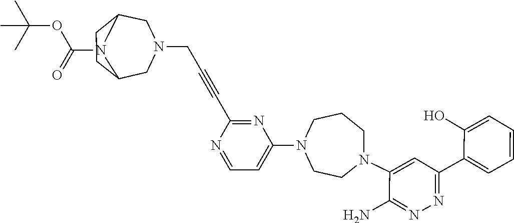 [SMILES: CC(C)(C)OC(=O)N1C2CCC1CN(CC#Cc1nccc(N3CCCN(c4cc(-c5ccccc5O)nnc4N)CC3)n1)C2] | tert-butyl 3-[3-(4-{4-[3-amino-6-(o- hydroxyphenyl)-4-pyridazinyl]-1,4- diazepan-1-yl}-2-pyrimidinyl)-2- propynyl]-3,8- diazabicyclo[3.2.1]octane-8- carboxylate |
| P-359 | 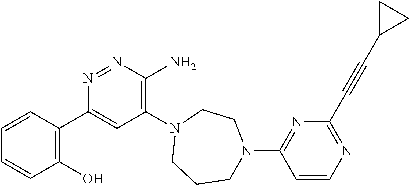 [SMILES: Nc1nnc(-c2ccccc2O)cc1N1CCCN(c2ccnc(C#CC3CC3)n2)CC1] | o-(6-amino-5-{4-[2-(2- cyclopropylethynyl)-4-pyrimidinyl]- 1,4-diazepan-1-yl}-3- pyridazinyl)phenol |
| P-360 | 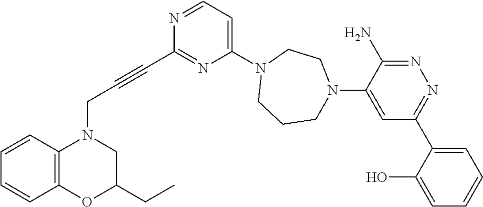 [SMILES: CCC1CN(CC#Cc2nccc(N3CCCN(c4cc(-c5ccccc5O)nnc4N)CC3)n2)c2ccccc2O1] | o-[6-amino-5-(4-{2-[3-(2-ethyl-3,4- dihydro-2H-1,4-benzoxazin-4-yl)-1- propynyl]-4-pyrimidinyl}-1,4- diazepan-1-yl)-3-pyridazinyl]phenol |
| P-361 | 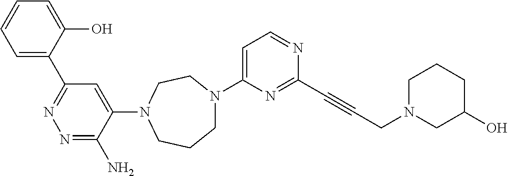 | 1-[3-(4-{4-[3-amino-6-(o- hydroxyphenyl)-4-pyridazinyl]-1,4- diazepan-1-yl}-2-pyrimidinyl)-2- propynyl]-3-piperidinol |
| P-362 | 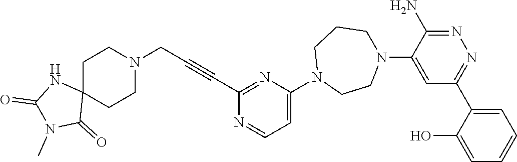 [SMILES: CN1C(=O)NC2(CCN(CC#Cc3nccc(N4CCCN(c5cc(-c6ccccc6O)nnc5N)CC4)n3)CC2)C1=O] | 8-[3-(4-{4-[3-amino-6-(o- hydroxyphenyl)-4-pyridazinyl]-1,4- diazepan-1-yl}-2-pyrimidinyl)-2- propynyl]-3-methyl-1,3,8-triaza-2,4- spiro[4.5]decanedione |
| P-363 | 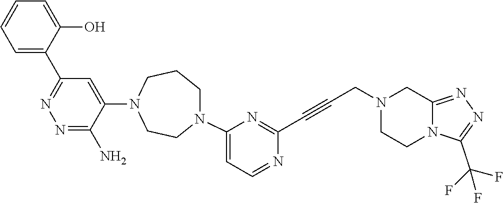 [SMILES: Nc1nnc(-c2ccccc2O)cc1N1CCCN(c2ccnc(C#CCN3CCn4c(nnc4C(F)(F)F)C3)n2)CC1] | o-{6-amino-5-[4-(2-{3-[3- (trifluoromethyl)-4,5,6,7-tetrahydro- 1,2,3a,6-tetraaza-6-indenyl]-1- propynyl}-4-pyrimidinyl)-1,4- diazepan-1-yl]-3-pyridazinyl}phenol |
| P-364 | 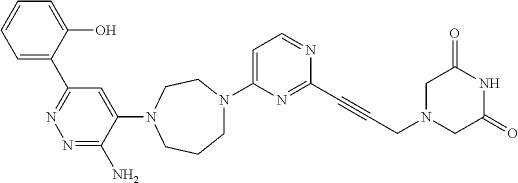 | 4-[3-(4-{4-[3-amino-6-(o- hydroxyphenyl)-4-pyridazinyl]-1,4- diazepan-1-yl}-2-pyrimidinyl)-2- propynyl]-2,6-piperazinedione |
| P-365 | 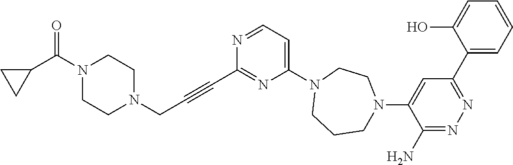 [SMILES: Nc1nnc(-c2ccccc2O)cc1N1CCCN(c2ccnc(C#CCN3CCN(C(=O)C4CC4)CC3)n2)CC1] | (4-[3-(4-{4-[3-amino-6-(o- hydroxyphenyl)-4-pyridazinyl]-1,4- diazepan-1-yl}-2-pyrimidinyl)-2- propynyl]-1- piperazinyl}cyclopropylmethanone |
| P-366 | 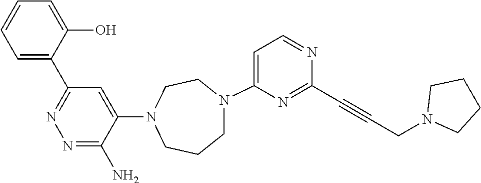 [SMILES: Nc1nnc(-c2ccccc2O)cc1N1CCCN(c2ccnc(C#CCN3CCCC3)n2)CC1] | o-[6-amino-5-(4-{2-[3-(1- pyrrolidinyl)-1-propynyl]-4- pyrimidinyl}-1,4-diazepan-1-yl)-3- pyridazinyl]phenol |
| P-367 | 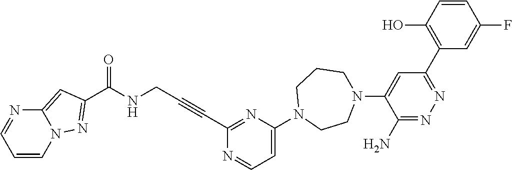 [SMILES: Nc1nnc(-c2cc(F)ccc2O)cc1N1CCCN(c2ccnc(C#CCNC(=O)c3cc4ncccn4n3)n2)CC1] | N-[3-(4-{4-[3-amino-6-(5-fluoro-2- hydroxyphenyl)-4-pyridazinyl]-1,4- diazepan-1-yl}-2-pyrimidinyl)-2- propynyl]-1,4,7a-triaza-2- indenecarboxamide |
| P-368 | 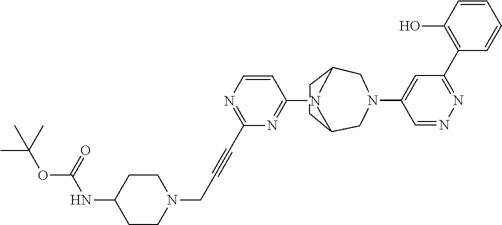 | tert-butyl-1-[3-(4-{3-[6-(o- hydroxyphenyl)-4-pyridazinyl]-3,8- diazabicyclo[3.2.1]oct-8-yl}-2- pyrimidinyl)-2-propynyl]-4- piperidylaminoformylate |
| P-369 | 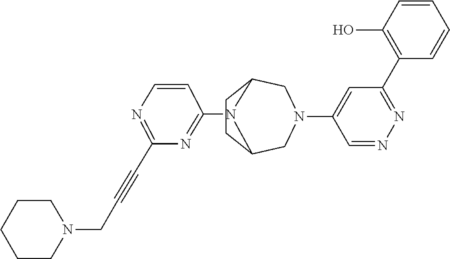 [SMILES: Oc1ccccc1-c1cc(N2CC3CCC(C2)N3c2ccnc(C#CCN3CCCCC3)n2)cnn1] | o-(5-{8-[2-(3-piperidino-1-propynyl)- 4-pyrimidinyl]-3,8- diazabicyclo[3.2.1]oct-3-yl}-3- pyridazinyl)phenol |
| P-370 | 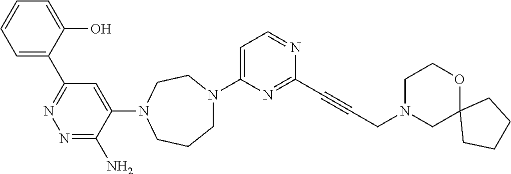 | o-{6-amino-5-[4-(2-{3-(6-oxa-9-aza- 9-spiro[4.5]decyl)-1-propynyl}-4- pyrimidinyl)-1,4-diazepan-1-yl]-3- pyridazinyl}phenol |
| P-371 | 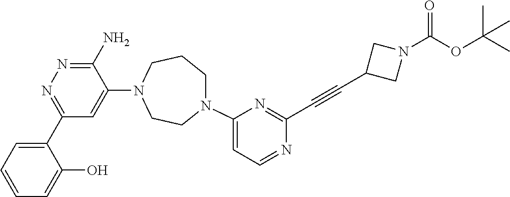 [SMILES: CC(C)(C)OC(=O)N1CC(C#Cc2nccc(N3CCCN(c4cc(-c5ccccc5O)nnc4N)CC3)n2)C1] | tert-butyl 3-[2-(4-{4-[3-amino-6-(o- hydroxyphenyl)-4-pyridazinyl]-1,4- diazepan-1-yl}-2- pyrimidinyl)ethynyl]-1- azetidinecarboxylate |
| P-372 | 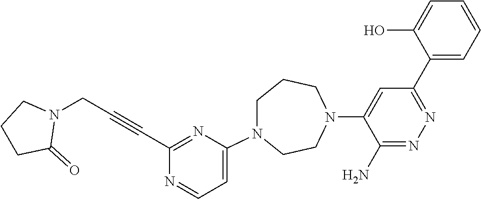 [SMILES: Nc1nnc(-c2ccccc2O)cc1N1CCCN(c2ccnc(C#CCN3CCCC3=O)n2)CC1] | 1-[3-(4-{4-[3-amino-6-(o- hydroxyphenyl)-4-pyridazinyl]-1,4- diazepan-1-yl}-2-pyrimidinyl)-2- propynyl]-2-pyrrolidinone |
| P-373 | 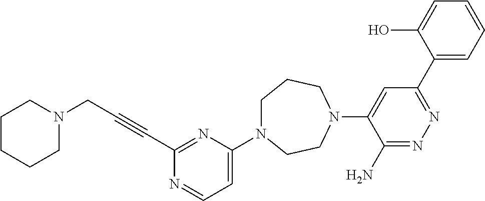 [SMILES: Nc1nnc(-c2ccccc2O)cc1N1CCCN(c2ccnc(C#CCN3CCCCC3)n2)CC1] | o-(6-amino-5-{4-[2-(3-piperidino-1- propynyl)-4-pyrimidinyl]-1,4- diazepan-1-yl}-3-pyridazinyl)phenol |
| P-374 | 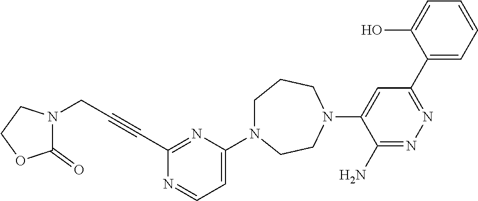 [SMILES: Nc1nnc(-c2ccccc2O)cc1N1CCCN(c2ccnc(C#CCN3CCOC3=O)n2)CC1] | 3-[3-(4-{4-[3-amino-6-(o- hydroxyphenyl)-4-pyridazinyl]-1,4- diazepan-1-yl}-2-pyrimidinyl)-2- propynyl]-1,3-oxazolidin-2-one |
| P-375 | 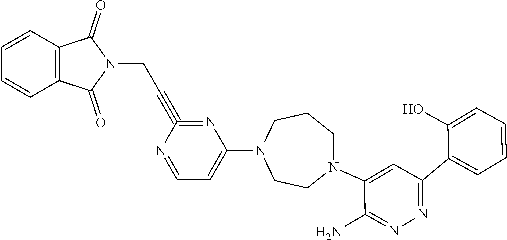 | 2-[3-(4-{4-[3-amino-6-(o- hydroxyphenyl)-4-pyridazinyl]-1,4- diazepan-1-yl}-2-pyrimidinyl)-2- propynyl]-1,3-isoindolinedione |
| P-376 | 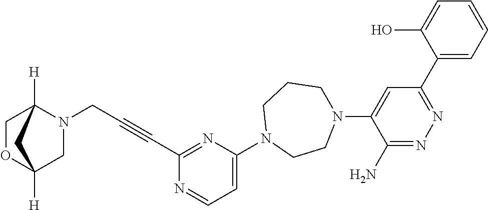 [SMILES: Nc1nnc(-c2ccccc2O)cc1N1CCCN(c2ccnc(C#CCN3C[C@H]4C[C@@H]3CO4)n2)CC1] | o-(5-{4-[2-(3-{(1R,4R)-2-oxa-5- azabicyclo[2.2.1]hept-5-yl}-1- propynyl)-4-pyrimidinyl]-1,4- diazepan-1-yl}-6-amino-3- pyridazinyl)phenol |
| P-377 | 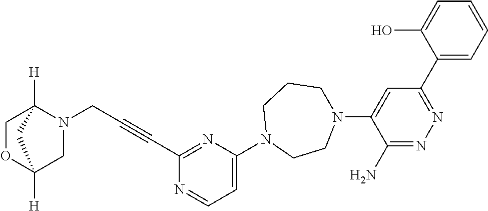 [SMILES: Nc1nnc(-c2ccccc2O)cc1N1CCCN(c2ccnc(C#CCN3C[C@@H]4C[C@H]3CO4)n2)CC1] | o-(5-{4-[2-(3-{(1S,4S)-2-oxa-5- azabicyclo[2.2.1]hept-5-yl}-1- propynyl)-4-pyrimidinyl]-1,4- diazepan-1-yl}-6-amino-3- pyridazinyl)phenol |
| P-378 | 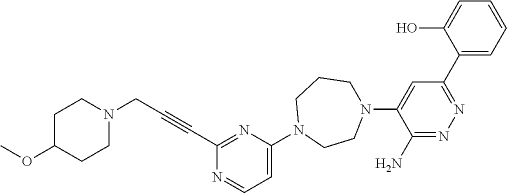 [SMILES: COC1CCN(CC#Cc2nccc(N3CCCN(c4cc(-c5ccccc5O)nnc4N)CC3)n2)CC1] | o-[6-amino-5-(4-{2-[3-(4-methoxy-1- piperidyl)-1-propynyl]-4- pyrimidinyl}-1,4-diazepan-1-yl)-3- pyridazinyl]phenol |
| P-379 | 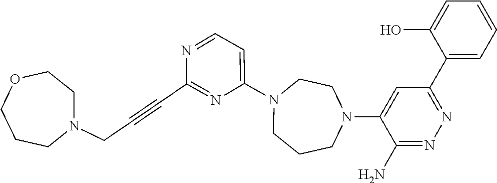 [SMILES: Nc1nnc(-c2ccccc2O)cc1N1CCCN(c2ccnc(C#CCN3CCCOCC3)n2)CC1] | o-[6-amino-5-(4-{2-[3-(1,4-oxazepan- 4-yl)-1-propynyl]-4-pyrimidinyl}-1,4- diazepan-1-yl)-3-pyridazinyl]phenol |
| P-380 | 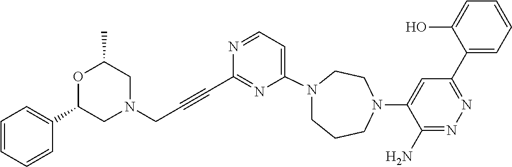 [SMILES: C[C@@H]1CN(CC#Cc2nccc(N3CCCN(c4cc(-c5ccccc5O)nnc4N)CC3)n2)C[C@H](c2ccccc2)O1] | o-{5-[4-(2-{3-[(2R,6S)-2-methyl-6- phenyl-4-morpholinyl]-1-propynyl}- 4-pyrimidinyl)-1,4-diazepan-1-yl]-6- amino-3-pyridazinyl}phenol |
| P-381 | 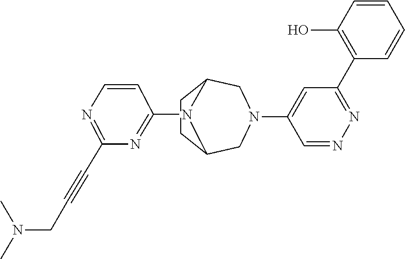 [SMILES: CN(C)CC#Cc1nccc(N2C3CCC2CN(c2cnnc(-c4ccccc4O)c2)C3)n1] | o-[5-(8-{2-[3-(dimethylamino)-1- propynyl]-4-pyrimidinyl}-3,8- diazabicyclo[3.2.1]oct-3-yl)-3- pyridazinyl]phenol |
| P-382 | 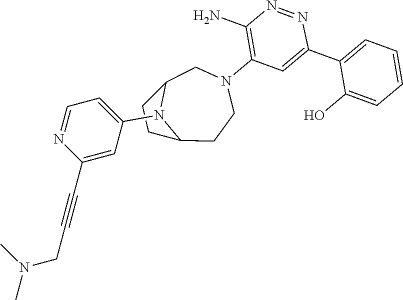 [SMILES: CN(C)CC#Cc1cc(N2C3CCC2CN(c2cc(-c4ccccc4O)nnc2N)CC3)ccn1] | o-[6-amino-5-(9-{2-[3- (dimethylamino)-1-propynyl]-4- pyridyl}-3,9-diazabicyclo[4.2.1]non- 3-yl)-3-pyridazinyl]phenol |
| P-383 | 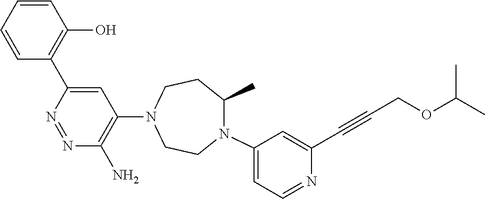 [SMILES: CC(C)OCC#Cc1cc(N2CCN(c3cc(-c4ccccc4O)nnc3N)CC[C@H]2C)ccn1] | o-(5-{(R)-4-[2-(3-isopropoxy-1- propynyl)-4-pyridyl]-5-methyl-1,4- diazepan-1-yl}-6-amino-3- pyridazinyl)phenol |
| P-384 | 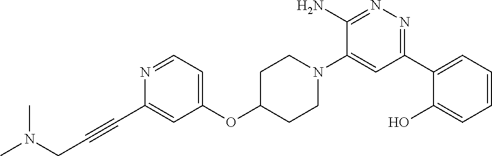 [SMILES: CN(C)CC#Cc1cc(OC2CCN(c3cc(-c4ccccc4O)nnc3N)CC2)ccn1] | o-[6-amino-5-(4-{2-[3- (dimethylamino)-1-propynyl]-4- pyridyloxy}-1-piperidyl)-3- pyridazinyl]phenol |
| P-385 | 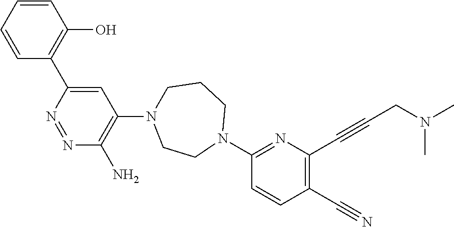 [SMILES: CN(C)CC#Cc1nc(N2CCCN(c3cc(-c4ccccc4O)nnc3N)CC2)ccc1C#N] | 6-{4-[3-amino-6-(o-hydroxyphenyl)- 4-pyridazinyl]-1,4-diazepan-1-yl}-2- [3-(dimethylamino)-1- propynyl]nicotinonitrile |
| P-386 | 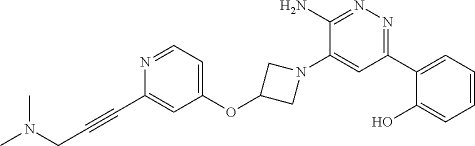 [SMILES: CN(C)CC#Cc1cc(OC2CN(c3cc(-c4ccccc4O)nnc3N)C2)ccn1] | o-[6-amino-5-(3-{2-[3- (dimethylamino)-1-propynyl]-4- pyridyloxy}-1-azetidinyl)-3- pyridazinyl]phenol |
| P-387 | 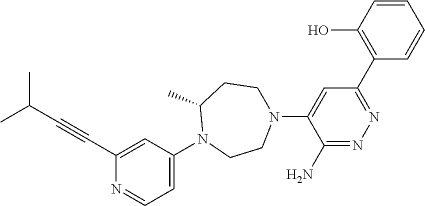 [SMILES: CC(C)C#Cc1cc(N2CCN(c3cc(-c4ccccc4O)nnc3N)CC[C@H]2C)ccn1] | o-(5-{(R)-5-methyl-4-[2-(3-methyl-1- butynyl)-4-pyridyl]-1,4-diazepan-1- yl}-6-amino-3-pyridazinyl)phenol |
| P-388 | 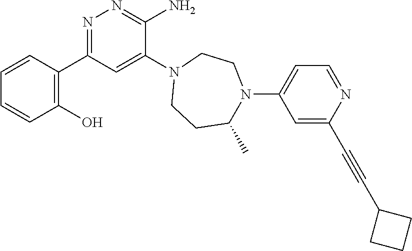 [SMILES: C[C@@H]1CCN(c2cc(-c3ccccc3O)nnc2N)CCN1c1ccnc(C#CC2CCC2)c1] | o-(5-{(R)-4-[2-(2-cyclobutylethynyl)- 4-pyridyl]-5-methyl-1,4-diazepan-1- yl}-6-amino-3-pyridazinyl)phenol |
| P-389 | 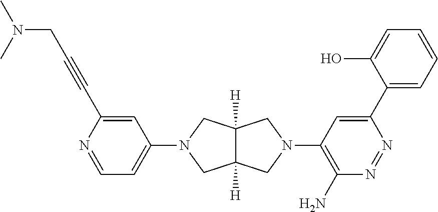 [SMILES: CN(C)CC#Cc1cc(N2C[C@@H]3CN(c4cc(-c5ccccc5O)nnc4N)C[C@@H]3C2)ccn1] | o-{5-[(1R,5S)-7-{2-[3- (dimethylamino)-1-propynyl]-4- pyridyl}-3,7-diazabicyclo[3.3.0]oct- 3-yl]-6-amino-3-pyridazinyl}phenol |
| P-390 | 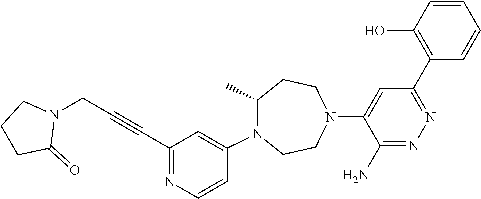 [SMILES: C[C@@H]1CCN(c2cc(-c3ccccc3O)nnc2N)CCN1c1ccnc(C#CCN2CCCC2=O)c1] | 1-[3-(4-{(R)-4-[3-amino-6-(o- hydroxyphenyl)-4-pyridazinyl]-7- methyl-1,4-diazepan-1-yl}-2- pyridyl)-2-propynyl]-2-pyrrolidinone |
| P-391 | 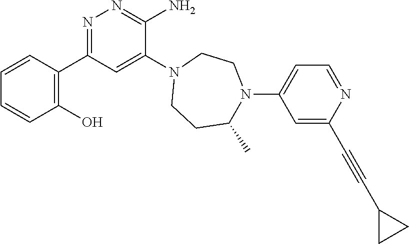 [SMILES: C[C@@H]1CCN(c2cc(-c3ccccc3O)nnc2N)CCN1c1ccnc(C#CC2CC2)c1] | o-(5-{(R)-4-[2-(2- cyclopropylethynyl)-4-pyridyl]-5- methyl-1,4-diazepan-1-yl}-6-amino- 3-pyridazinyl)phenol |
| P-392 | 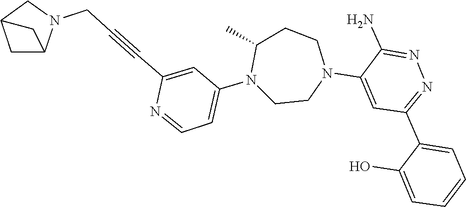 | o-{5-[(R)-4-(2-{3-(2- azabicyclo[2.1.1]hex-2-yl)-1- propynyl}-4-pyridyl)-5-methyl-1,4- diazepan-1-yl]-6-amino-3- pyridazinyl}phenol |
| P-393 | 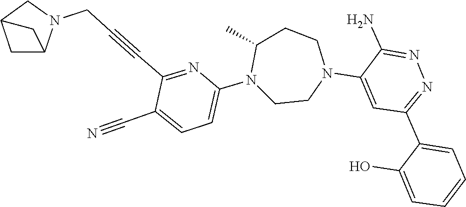 | 6-{(R)-4-[3-amino-6-(o- hydroxyphenyl)-4-pyridazinyl]-7- methyl-1,4-diazepan-1-yl}-2-{3-(2- azabicyclo[2.1.1]hex-2-yl)-1- propynyl}nicotinonitrile |
| P-394 | 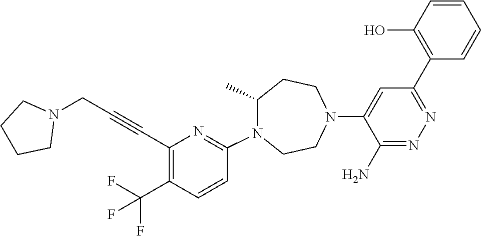 [SMILES: C[C@@H]1CCN(c2cc(-c3ccccc3O)nnc2N)CCN1c1ccc(C(F)(F)F)c(C#CCN2CCCC2)n1] | o-{5-[(R)-5-methyl-4-{6-[3-(1- pyrrolidinyl)-1-propynyl]-5- (trifluoromethyl)-2-pyridyl}-1,4- diazepan-1-yl]-6-amino-3- pyridazinyl}phenol |
| P-395 | 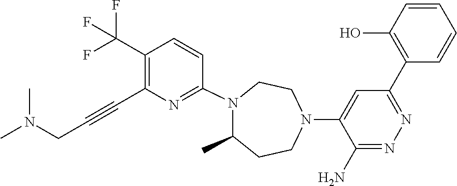 [SMILES: C[C@@H]1CCN(c2cc(-c3ccccc3O)nnc2N)CCN1c1ccc(C(F)(F)F)c(C#CCN(C)C)n1] | o-{5-[(R)-4-{6-[3-(dimethylamino)-1- propynyl]-5-(trifluoromethyl)-2- pyridyl}-5-methyl-1,4-diazepan-1- yl]-6-amino-3-pyridazinyl}phenol |
| P-396 | 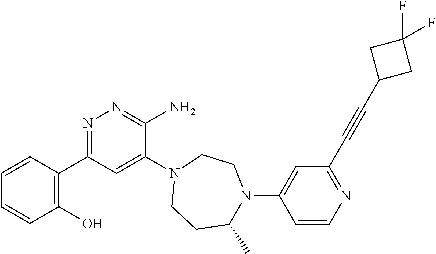 [SMILES: C[C@@H]1CCN(c2cc(-c3ccccc3O)nnc2N)CCN1c1ccnc(C#CC2CC(F)(F)C2)c1] | o-{5-[(R)-4-{2-[2-(3,3- difluorocyclobutyl)ethynyl]-4- pyridyl}-5-methyl-1,4-diazepan-1- yl]-6-amino-3-pyridazinyl}phenol |
| P-397 | 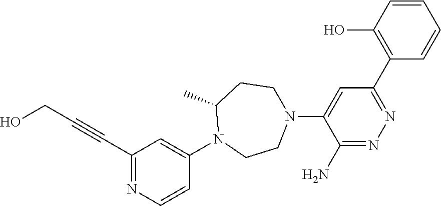 [SMILES: C[C@@H]1CCN(c2cc(-c3ccccc3O)nnc2N)CCN1c1ccnc(C#CCO)c1] | o-(5-{(R)-4-[2-(3-hydroxy-1- propynyl)-4-pyridyl]-5-methyl-1,4- diazepan-1-yl}-6-amino-3- pyridazinyl)phenol |
| P-398 | 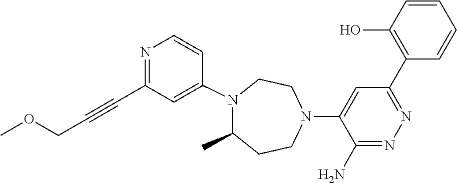 [SMILES: COCC#Cc1cc(N2CCN(c3cc(-c4ccccc4O)nnc3N)CC[C@H]2C)ccn1] | o-(5-{(R)-4-[2-(3-methoxy-1- propynyl)-4-pyridyl]-5-methyl-1,4- diazepan-1-yl}-6-amino-3- pyridazinyl)phenol |
| P-399 | 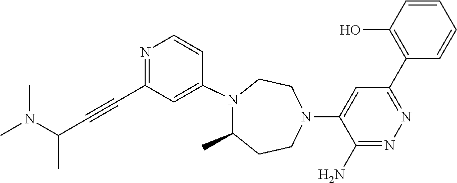 [SMILES: CC(C#Cc1cc(N2CCN(c3cc(-c4ccccc4O)nnc3N)CC[C@H]2C)ccn1)N(C)C] | o-{5-[(R)-4-{2-[3-(dimethylamino)-1- butynyl]-4-pyridyl}-5-methyl-1,4- diazepan-1-yl]-6-amino-3- pyridazinyl}phenol |
| P-400 | 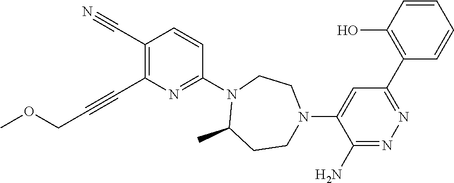 [SMILES: COCC#Cc1nc(N2CCN(c3cc(-c4ccccc4O)nnc3N)CC[C@H]2C)ccc1C#N] | 6-{(R)-4-[3-amino-6-(o- hydroxyphenyl)-4-pyridazinyl]-7- methyl-1,4-diazepan-1-yl}-2-(3- methoxy-1-propynyl)nicotinonitrile |
| P-401 | 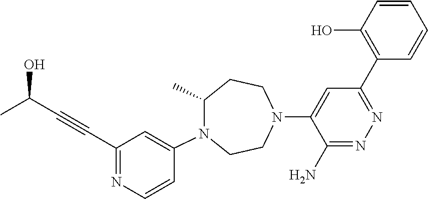 [SMILES: C[C@@H]1CCN(c2cc(-c3ccccc3O)nnc2N)CCN1c1ccnc(C#C[C@@H](C)O)c1] | o-{5-[(R)-4-{2-[(R)-3-hydroxy-1- butynyl]-4-pyridyl}-5-methyl-1,4- diazepan-1-yl]-6-amino-3- pyridazinyl}phenol |
| P-402 | 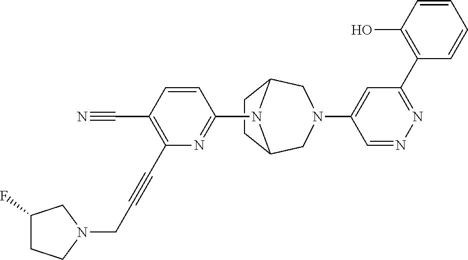 [SMILES: N#Cc1ccc(N2C3CCC2CN(c2cnnc(-c4ccccc4O)c2)C3)nc1C#CCN1CC[C@H](F)C1] | 2-{3-[(S)-3-fluoro-1-pyrrolidinyl]-1- propynyl}-6-{3-[6-(o- hydroxyphenyl)-4-pyridazinyl]-3,8- diazabicyclo[3.2.1]oct-8- yl}nicotinonitrile |
| P-403 | 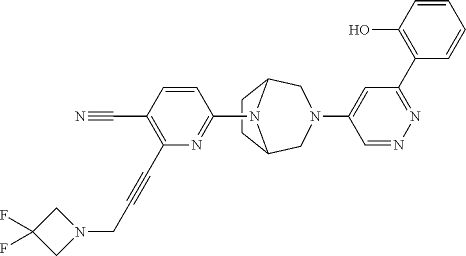 [SMILES: N#Cc1ccc(N2C3CCC2CN(c2cnnc(-c4ccccc4O)c2)C3)nc1C#CCN1CC(F)(F)C1] | 2-[3-(3,3-difluoro-1-azetidinyl)-1- propynyl]-6-{3-[6-(o- hydroxyphenyl)-4-pyridazinyl]-3,8- diazabicyclo[3.2.1]oct-8- yl}nicotinonitrile |
| P-404 | 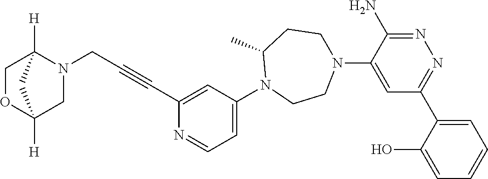 [SMILES: C[C@@H]1CCN(c2cc(-c3ccccc3O)nnc2N)CCN1c1ccnc(C#CCN2C[C@@H]3C[C@H]2CO3)c1] | o-{5-[(R)-4-[2-(3-{(1S,4S)-2-oxa-5- azabicyclo[2.2.1]hept-5-yl}-1- propynyl)-4-pyridyl]-5-methyl-1,4- diazepan-1-yl]-6-amino-3- pyridazinyl}phenol |
| P-405 | 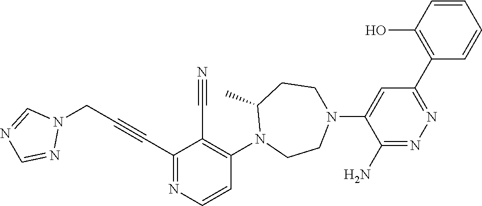 [SMILES: C[C@@H]1CCN(c2cc(-c3ccccc3O)nnc2N)CCN1c1ccnc(C#CCn2cncn2)c1C#N] | 4-{(R)-4-[3-amino-6-(o- hydroxyphenyl)-4-pyridazinyl]-7- methyl-1,4-diazepan-1-yl}-2-[3-(1H- 1,2,4-triazol-1-yl)-1- propynyl]nicotinonitrile |
| P-406 | 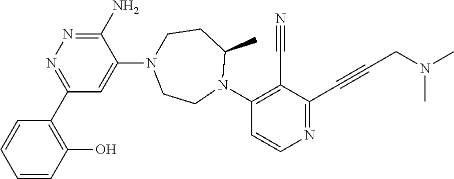 [SMILES: C[C@@H]1CCN(c2cc(-c3ccccc3O)nnc2N)CCN1c1ccnc(C#CCN(C)C)c1C#N] | 4-{(R)-4-[3-amino-6-(o- hydroxyphenyl)-4-pyridazinyl]-7- methyl-1,4-diazepan-1-yl}-2-[3- (dimethylamino)-1- propynyl]nicotinonitrile |
| P-407 | 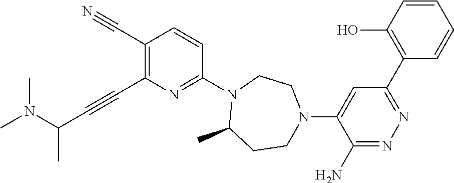 [SMILES: CC(C#Cc1nc(N2CCN(c3cc(-c4ccccc4O)nnc3N)CC[C@H]2C)ccc1C#N)N(C)C] | 6-{(R)-4-[3-amino-6-(o- hydroxyphenyl)-4-pyridazinyl]-7- methyl-1,4-diazepan-1-yl}-2-[3- (dimethylamino)-1- butynyl]nicotinonitrile |
| P-408 | 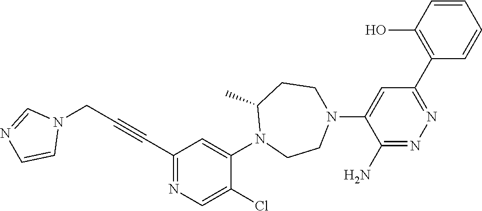 [SMILES: C[C@@H]1CCN(c2cc(-c3ccccc3O)nnc2N)CCN1c1cc(C#CCn2ccnc2)ncc1Cl] | o-{5-[(R)-4-{5-chloro-2-[3-(1- imidazolyl)-1-propynyl]-4-pyridyl}- 5-methyl-1,4-diazepan-1-yl]-6-amino- 3-pyridazinyl}phenol |
| P-409 | 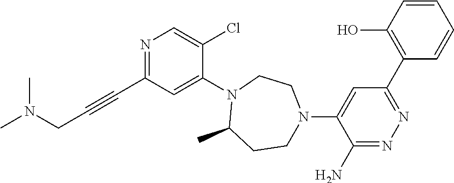 [SMILES: C[C@@H]1CCN(c2cc(-c3ccccc3O)nnc2N)CCN1c1cc(C#CCN(C)C)ncc1Cl] | o-{5-[(R)-4-{5-chloro-2-[3- (dimethylamino)-1-propynyl]-4- pyridyl}-5-methyl-1,4-diazepan-1- yl]-6-amino-3-pyridazinyl}phenol |
| P-410 | 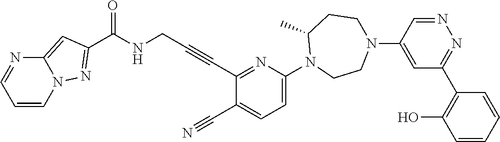 [SMILES: C[C@@H]1CCN(c2cnnc(-c3ccccc3O)c2)CCN1c1ccc(C#N)c(C#CCNC(=O)c2cc3ncccn3n2)n1] | N-[3-(6-{(R)-4-[6-(o- hydroxyphenyl)-4-pyridazinyl]-7- methyl-1,4-diazepan-1-yl}-3-cyano- 2-pyridyl)-2-propynyl]-1,4,7a-triaza- 2-indenecarboxamide |
| P-411 | 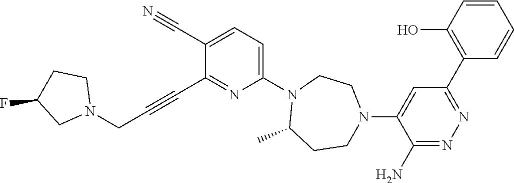 [SMILES: C[C@H]1CCN(c2cc(-c3ccccc3O)nnc2N)CCN1c1ccc(C#N)c(C#CCN2CC[C@H](F)C2)n1] | 6-{(S)-4-[3-amino-6-(o- hydroxyphenyl)-4-pyridazinyl]-7- methyl-1,4-diazepan-1-yl}-2-{3-[(S)- 3-fluoro-1-pyrrolidinyl]-1- propynyl}nicotinonitrile |
| P-412 | 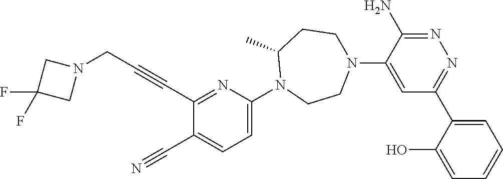 [SMILES: C[C@@H]1CCN(c2cc(-c3ccccc3O)nnc2N)CCN1c1ccc(C#N)c(C#CCN2CC(F)(F)C2)n1] | 6-{(R)-4-[3-amino-6-(o- hydroxyphenyl)-4-pyridazinyl]-7- methyl-1,4-diazepan-1-yl}-2-[3-(3,3- difluoro-1-azetidinyl)-1- propynyl]nicotinonitrile |
| P-413 | 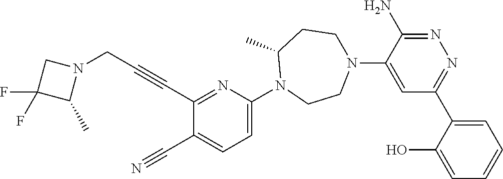 [SMILES: C[C@@H]1CCN(c2cc(-c3ccccc3O)nnc2N)CCN1c1ccc(C#N)c(C#CCN2CC(F)(F)[C@H]2C)n1] | 6-{(R)-4-[3-amino-6-(o- hydroxyphenyl)-4-pyridazinyl]-7- methyl-1,4-diazepan-1-yl}-2-{3-[(R)- 3,3-difluoro-2-methyl-1-azetidinyl]-1- propynyl}nicotinonitrile |
| P-414 | 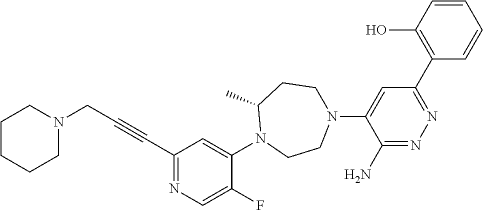 [SMILES: C[C@@H]1CCN(c2cc(-c3ccccc3O)nnc2N)CCN1c1cc(C#CCN2CCCCC2)ncc1F] | o-(5-{(R)-4-[5-fluoro-2-(3-piperidino- 1-propynyl)-4-pyridyl]-5-methyl-1,4- diazepan-1-yl}-6-amino-3- pyridazinyl)phenol |
| P-415 | 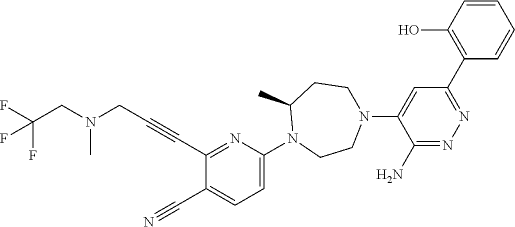 [SMILES: C[C@H]1CCN(c2cc(-c3ccccc3O)nnc2N)CCN1c1ccc(C#N)c(C#CCN(C)CC(F)(F)F)n1] | 2-{3-[N-methyl(2,2,2- trifluoroethyl)amino]-1-propynyl}-6- {(S)-4-[3-amino-6-(o- hydroxyphenyl)-4-pyridazinyl]-7- methyl-1,4-diazepan-1- yl}nicotinonitrile |
| P-416 | 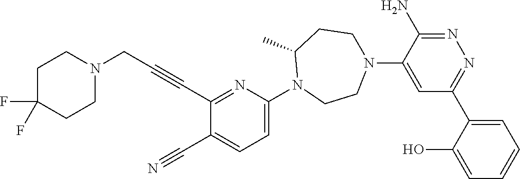 [SMILES: C[C@@H]1CCN(c2cc(-c3ccccc3O)nnc2N)CCN1c1ccc(C#N)c(C#CCN2CCC(F)(F)CC2)n1] | 6-{(R)-4-[3-amino-6-(o- hydroxyphenyl)-4-pyridazinyl]-7- methyl-1,4-diazepan-1-yl}-2-[3-(4,4- difluoro-1-piperidyl)-1- propynyl]nicotinonitrile |
| P-417 | 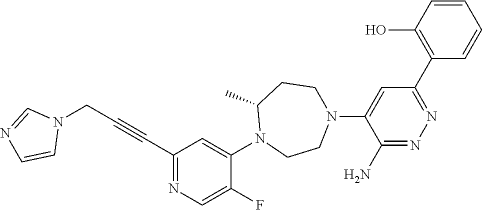 [SMILES: C[C@@H]1CCN(c2cc(-c3ccccc3O)nnc2N)CCN1c1cc(C#CCn2ccnc2)ncc1F] | o-{5-[(R)-4-{5-fluoro-2-[3-(1- imidazolyl)-1-propynyl]-4-pyridyl}- 5-methyl-1,4-diazepan-1-yl]-6-amino- 3-pyridazinyl}phenol |
| P-418 | 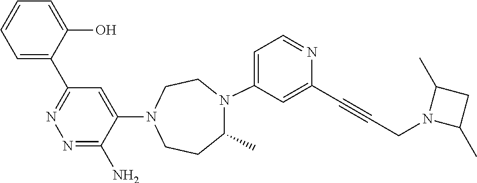 [SMILES: CC1CC(C)N1CC#Cc1cc(N2CCN(c3cc(-c4ccccc4O)nnc3N)CC[C@H]2C)ccn1] | o-{5-[(R)-4-{2-[3-(2,4-dimethyl-1- azetidinyl)-1-propynyl]-4-pyridyl}-5- methyl-1,4-diazepan-1-yl]-6-amino-3- pyridazinyl}phenol |
| P-419 | 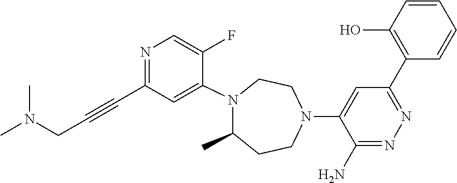 [SMILES: C[C@@H]1CCN(c2cc(-c3ccccc3O)nnc2N)CCN1c1cc(C#CCN(C)C)ncc1F] | o-{5-[(R)-4-{2-[3-(dimethylamino)-1- propynyl]-5-fluoro-4-pyridyl}-5- methyl-1,4-diazepan-1-yl]-6-amino-3- pyridazinyl}phenol |
| P-420 | 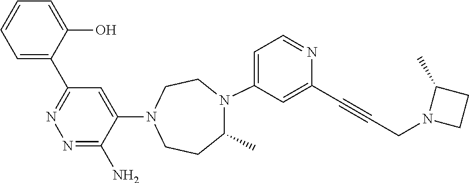 [SMILES: C[C@@H]1CCN1CC#Cc1cc(N2CCN(c3cc(-c4ccccc4O)nnc3N)CC[C@H]2C)ccn1] | o-{5-[(R)-4-(2-{3-[(R)-2-methyl-1- azetidinyl]-1-propynyl}-4-pyridyl)-5- methyl-1,4-diazepan-1-yl]-6-amino-3- pyridazinyl}phenol |
| P-421 | 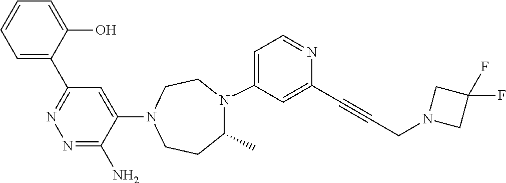 [SMILES: C[C@@H]1CCN(c2cc(-c3ccccc3O)nnc2N)CCN1c1ccnc(C#CCN2CC(F)(F)C2)c1] | o-{5-[(R)-4-{2-[3-(3,3-difluoro-1- azetidinyl)-1-propynyl]-4-pyridyl}-5- methyl-1,4-diazepan-1-yl]-6-amino-3- pyridazinyl}phenol |
| P-422 | 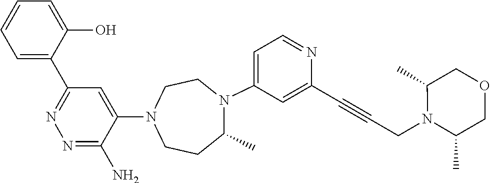 [SMILES: C[C@@H]1CCN(c2cc(-c3ccccc3O)nnc2N)CCN1c1ccnc(C#CCN2[C@H](C)COC[C@@H]2C)c1] | o-{5-[(R)-4-(2-{3-[(3R,5S)-3,5- dimethyl-4-morpholinyl]-1- propynyl}-4-pyridyl)-5-methyl-1,4- diazepan-1-yl]-6-amino-3- pyridazinyl}phenol |
| P-423 | 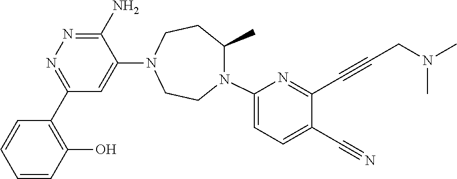 [SMILES: C[C@@H]1CCN(c2cc(-c3ccccc3O)nnc2N)CCN1c1ccc(C#N)c(C#CCN(C)C)n1] | 6-{(R)-4-[3-amino-6-(o- hydroxyphenyl)-4-pyridazinyl]-7- methyl-1,4-diazepan-1-yl}-2-[3- (dimethylamino)-1- propynyl]nicotinonitrile |
| P-424 | 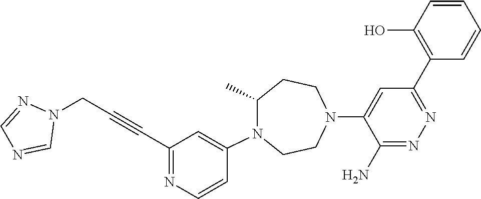 [SMILES: C[C@@H]1CCN(c2cc(-c3ccccc3O)nnc2N)CCN1c1ccnc(C#CCn2cncn2)c1] | o-{5-[(R)-5-methyl-4-{2-[3-(1H- 1,2,4-triazol-1-yl)-1-propynyl]-4- pyridyl}-1,4-diazepan-1-yl]-6-amino- 3-pyridazinyl}phenol |
| P-425 | 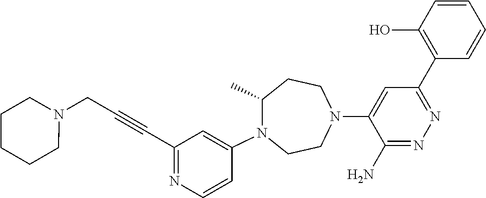 [SMILES: C[C@@H]1CCN(c2cc(-c3ccccc3O)nnc2N)CCN1c1ccnc(C#CCN2CCCCC2)c1] | o-(5-{(R)-5-methyl-4-[2-(3- piperidino-1-propynyl)-4-pyridyl]- 1,4-diazepan-1-yl}-6-amino-3- pyridazinyl)phenol |
| P-426 | 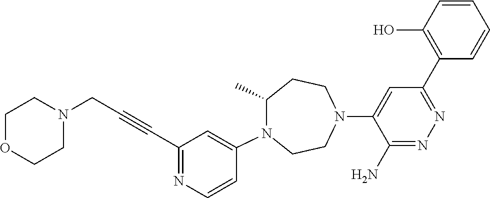 [SMILES: C[C@@H]1CCN(c2cc(-c3ccccc3O)nnc2N)CCN1c1ccnc(C#CCN2CCOCC2)c1] | o-(5-{(R)-5-methyl-4-[2-(3- morpholino-1-propynyl)-4-pyridyl]- 1,4-diazepan-1-yl}-6-amino-3- pyridazinyl)phenol |
| P-427 | 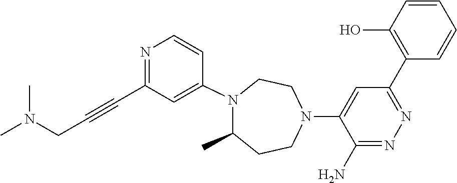 [SMILES: C[C@@H]1CCN(c2cc(-c3ccccc3O)nnc2N)CCN1c1ccnc(C#CCN(C)C)c1] | o-{5-[(R)-4-{2-[3-(dimethylamino)-1- propynyl]-4-pyridyl}-5-methyl-1,4- diazepan-1-yl]-6-amino-3- pyridazinyl}phenol |
| P-428 | 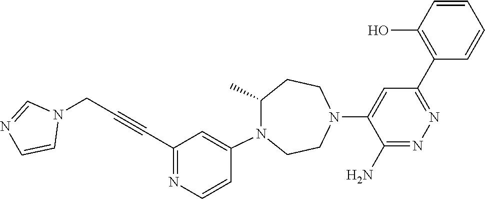 [SMILES: C[C@@H]1CCN(c2cc(-c3ccccc3O)nnc2N)CCN1c1ccnc(C#CCn2ccnc2)c1] | o-{5-[(R)-4-{2-[3-(1-imidazolyl)-1- propynyl]-4-pyridyl}-5-methyl-1,4- diazepan-1-yl]-6-amino-3- pyridazinyl}phenol |
| P-429 | 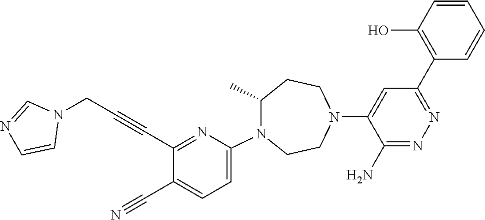 [SMILES: C[C@@H]1CCN(c2cc(-c3ccccc3O)nnc2N)CCN1c1ccc(C#N)c(C#CCn2ccnc2)n1] | 6-{(R)-4-[3-amino-6-(o- hydroxyphenyl)-4-pyridazinyl]-7- methyl-1,4-diazepan-1-yl}-2-[3-(1- imidazolyl)-1- propynyl]nicotinonitrile |
| P-430 | 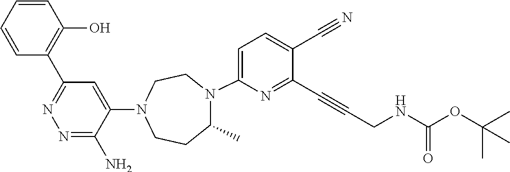 [SMILES: C[C@@H]1CCN(c2cc(-c3ccccc3O)nnc2N)CCN1c1ccc(C#N)c(C#CCNC(=O)OC(C)(C)C)n1] | 3-(6-{(R)-4-[3-amino-6-(o- hydroxyphenyl)-4-pyridazinyl]-7- methyl-1,4-diazepan-1-yl}-3-cyano- 2-pyridyl)-2-propynyl 2-methyl-2- propanecarbamate |
| P-431 | 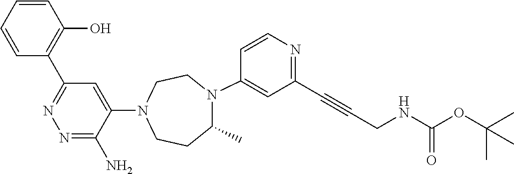 [SMILES: C[C@@H]1CCN(c2cc(-c3ccccc3O)nnc2N)CCN1c1ccnc(C#CCNC(=O)OC(C)(C)C)c1] | 3-(4-{(R)-4-[3-amino-6-(o- hydroxyphenyl)-4-pyridazinyl]-7- methyl-1,4-diazepan-1-yl}-2- pyridyl)-2-propynyl 2-methyl-2- propanecarbamate |
| P-432 | 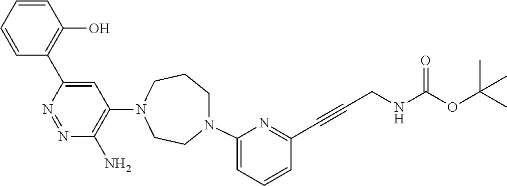 [SMILES: CC(C)(C)OC(=O)NCC#Cc1cccc(N2CCCN(c3cc(-c4ccccc4O)nnc3N)CC2)n1] | 3-(6-{4-[3-amino-6-(o- hydroxyphenyl)-4-pyridazinyl]-1,4- diazepan-1-yl}-2-pyridyl)-2-propynyl 2-methyl-2-propanecarbamate |

Representative and non-limiting examples of compounds within the scope of Formula i or Formula I′ above are set forth in Table 2, where each of which include their pharmaceutically acceptable salts, solvates, hydrates, or tautomers thereof.

TABLE 2

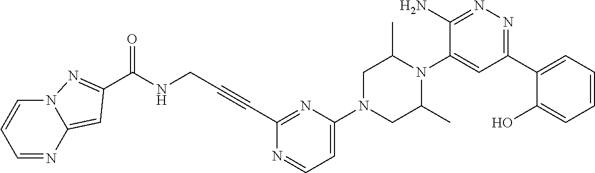 [SMILES: CC1CN(c2ccnc(C#CCNC(=O)c3cc4ncccn4n3)n2)CC(C)N1c1cc(-c2ccccc2O)nnc1N]

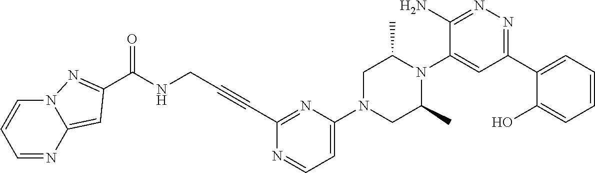 [SMILES: C[C@H]1CN(c2ccnc(C#CCNC(=O)c3cc4ncccn4n3)n2)C[C@H](C)N1c1cc(-c2ccccc2O)nnc1N]

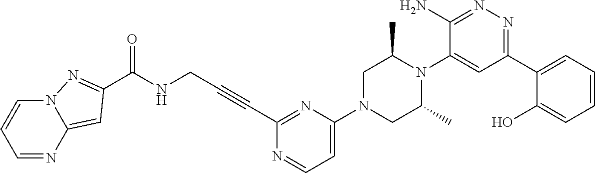 [SMILES: C[C@@H]1CN(c2ccnc(C#CCNC(=O)c3cc4ncccn4n3)n2)C[C@@H](C)N1c1cc(-c2ccccc2O)nnc1N]

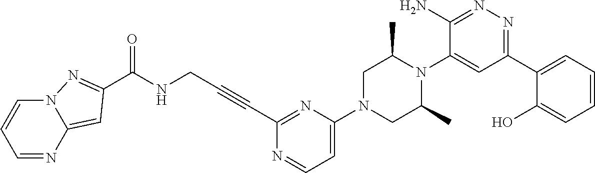 [SMILES: C[C@@H]1CN(c2ccnc(C#CCNC(=O)c3cc4ncccn4n3)n2)C[C@H](C)N1c1cc(-c2ccccc2O)nnc1N]

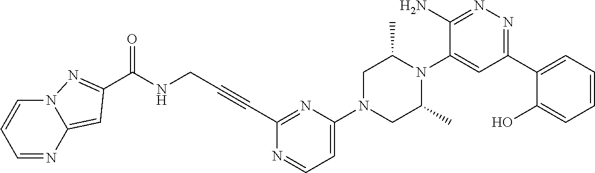 [SMILES: C[C@@H]1CN(c2ccnc(C#CCNC(=O)c3cc4ncccn4n3)n2)C[C@H](C)N1c1cc(-c2ccccc2O)nnc1N]

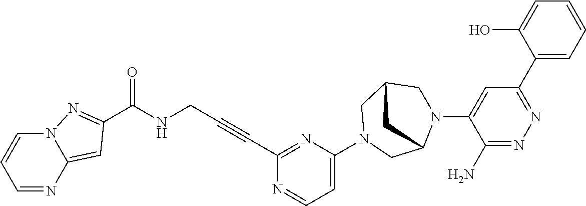 [SMILES: Nc1nnc(-c2ccccc2O)cc1N1C[C@@H]2C[C@H]1CN(c1ccnc(C#CCNC(=O)c3cc4ncccn4n3)n1)C2]

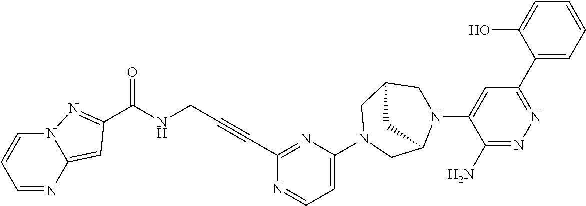 [SMILES: Nc1nnc(-c2ccccc2O)cc1N1C[C@H]2C[C@@H]1CN(c1ccnc(C#CCNC(=O)c3cc4ncccn4n3)n1)C2]

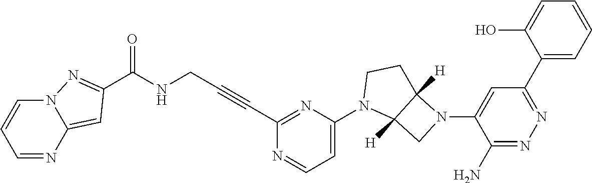 [SMILES: Nc1nnc(-c2ccccc2O)cc1N1C[C@H]2[C@@H]1CCN2c1ccnc(C#CCNC(=O)c2cc3ncccn3n2)n1]

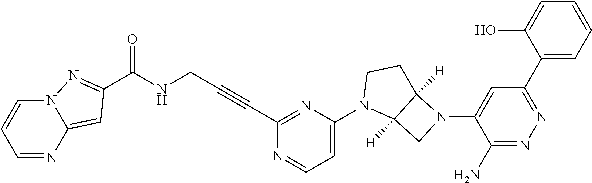 [SMILES: Nc1nnc(-c2ccccc2O)cc1N1C[C@@H]2[C@H]1CCN2c1ccnc(C#CCNC(=O)c2cc3ncccn3n2)n1]

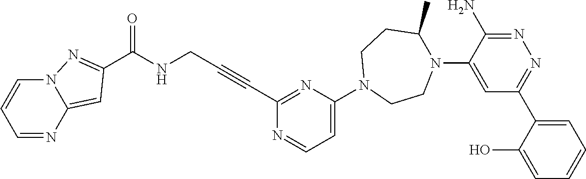 [SMILES: C[C@@H]1CCN(c2ccnc(C#CCNC(=O)c3cc4ncccn4n3)n2)CCN1c1cc(-c2ccccc2O)nnc1N]

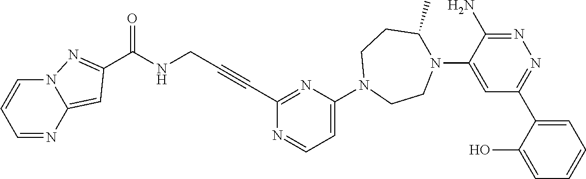 [SMILES: C[C@H]1CCN(c2ccnc(C#CCNC(=O)c3cc4ncccn4n3)n2)CCN1c1cc(-c2ccccc2O)nnc1N]

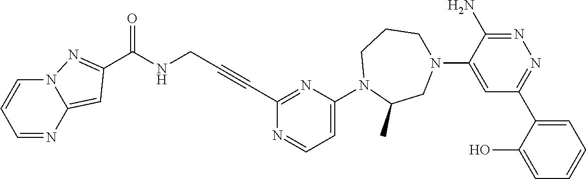 [SMILES: C[C@@H]1CN(c2cc(-c3ccccc3O)nnc2N)CCCN1c1ccnc(C#CCNC(=O)c2cc3ncccn3n2)n1]

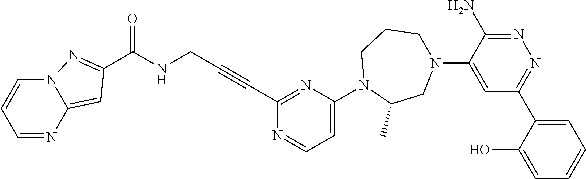 [SMILES: C[C@H]1CN(c2cc(-c3ccccc3O)nnc2N)CCCN1c1ccnc(C#CCNC(=O)c2cc3ncccn3n2)n1]

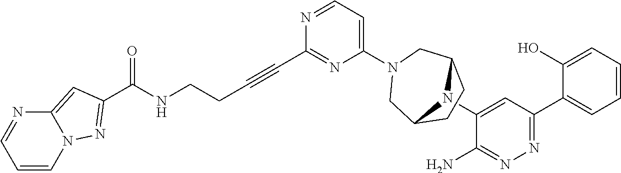 [SMILES: Nc1nnc(-c2ccccc2O)cc1N1[C@@H]2CC[C@H]1CN(c1ccnc(C#CCCNC(=O)c3cc4ncccn4n3)n1)C2]

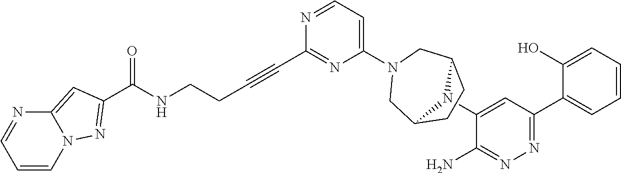 [SMILES: Nc1nnc(-c2ccccc2O)cc1N1[C@@H]2CC[C@H]1CN(c1ccnc(C#CCCNC(=O)c3cc4ncccn4n3)n1)C2]

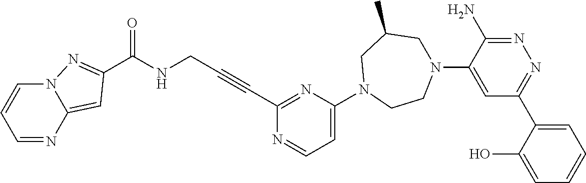 [SMILES: C[C@@H]1CN(c2ccnc(C#CCNC(=O)c3cc4ncccn4n3)n2)CCN(c2cc(-c3ccccc3O)nnc2N)C1]

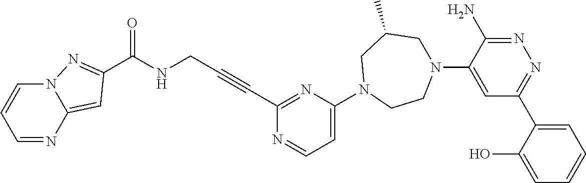 [SMILES: C[C@H]1CN(c2ccnc(C#CCNC(=O)c3cc4ncccn4n3)n2)CCN(c2cc(-c3ccccc3O)nnc2N)C1]

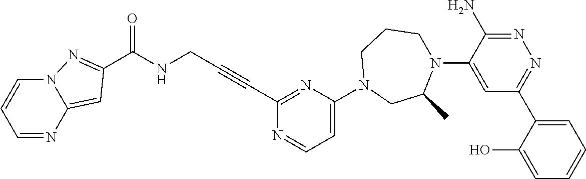 [SMILES: C[C@H]1CN(c2ccnc(C#CCNC(=O)c3cc4ncccn4n3)n2)CCCN1c1cc(-c2ccccc2O)nnc1N]

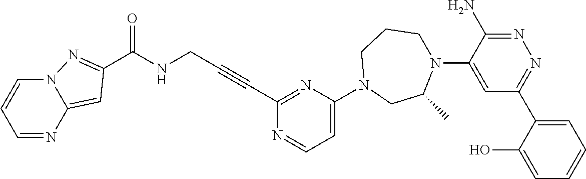 [SMILES: C[C@@H]1CN(c2ccnc(C#CCNC(=O)c3cc4ncccn4n3)n2)CCCN1c1cc(-c2ccccc2O)nnc1N]

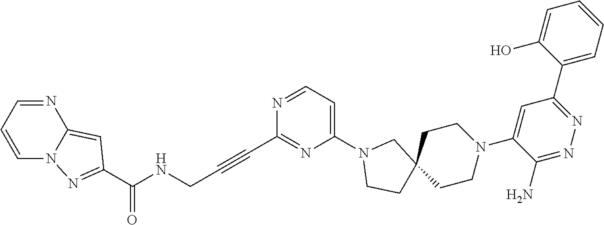

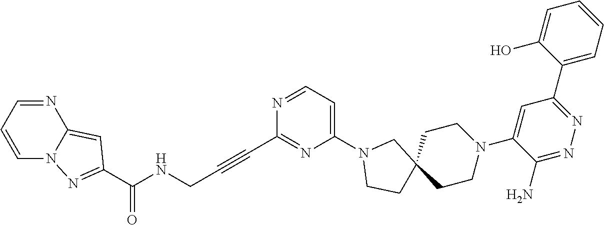

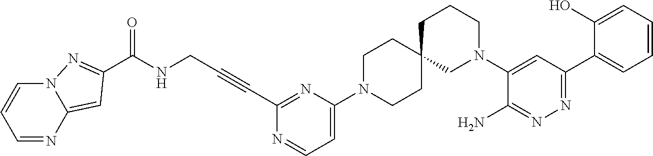

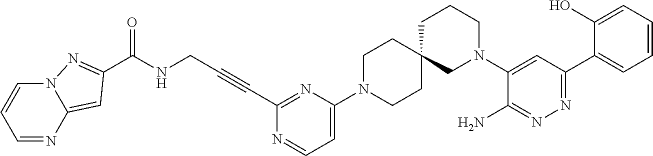

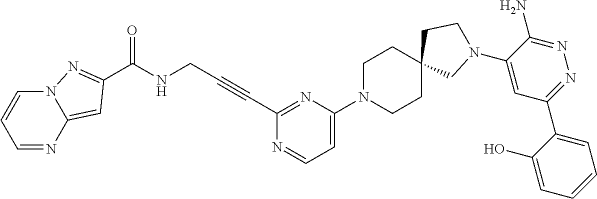

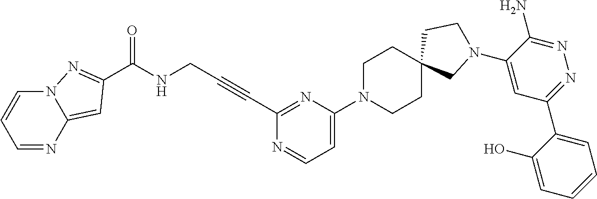

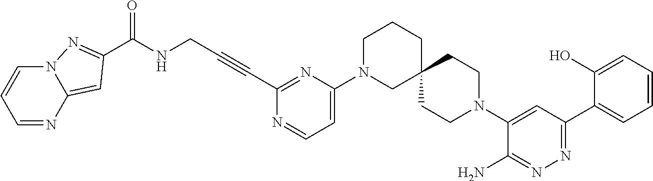

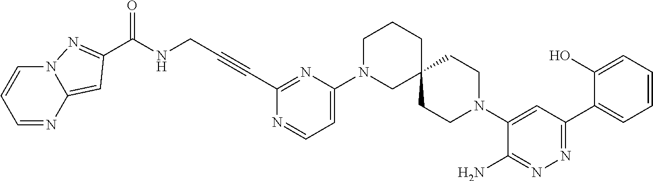

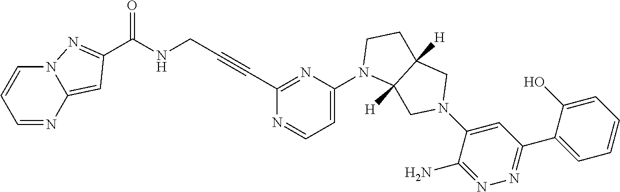 [SMILES: Nc1nnc(-c2ccccc2O)cc1N1C[C@H]2CCN(c3ccnc(C#CCNC(=O)c4cc5ncccn5n4)n3)[C@H]2C1]

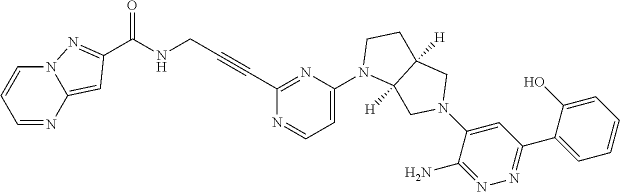 [SMILES: Nc1nnc(-c2ccccc2O)cc1N1C[C@@H]2CCN(c3ccnc(C#CCNC(=O)c4cc5ncccn5n4)n3)[C@@H]2C1]

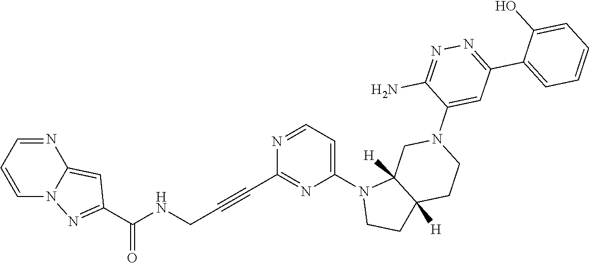 [SMILES: Nc1nnc(-c2ccccc2O)cc1N1CC[C@@H]2CCN(c3ccnc(C#CCNC(=O)c4cc5ncccn5n4)n3)[C@@H]2C1]

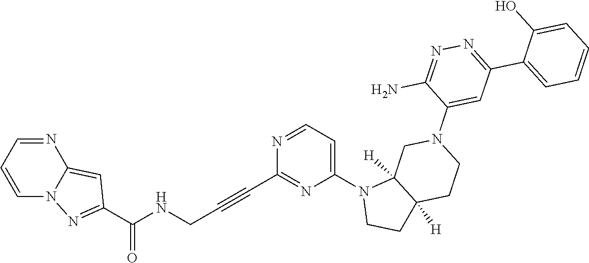 [SMILES: Nc1nnc(-c2ccccc2O)cc1N1CC[C@H]2CCN(c3ccnc(C#CCNC(=O)c4cc5ncccn5n4)n3)[C@H]2C1]

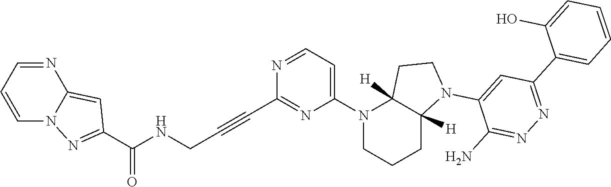 [SMILES: Nc1nnc(-c2ccccc2O)cc1N1CC[C@@H]2[C@H]1CCCN2c1ccnc(C#CCNC(=O)c2cc3ncccn3n2)n1]

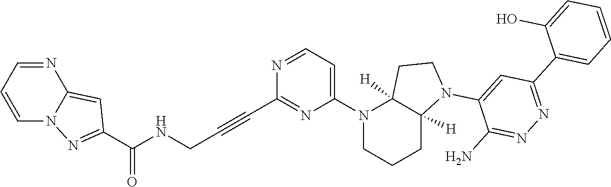 [SMILES: Nc1nnc(-c2ccccc2O)cc1N1CC[C@H]2[C@@H]1CCCN2c1ccnc(C#CCNC(=O)c2cc3ncccn3n2)n1]

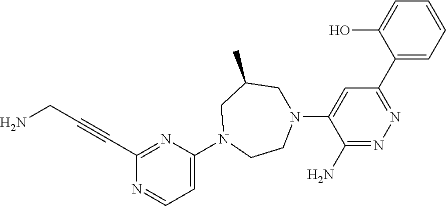 [SMILES: C[C@@H]1CN(c2ccnc(C#CCN)n2)CCN(c2cc(-c3ccccc3O)nnc2N)C1]

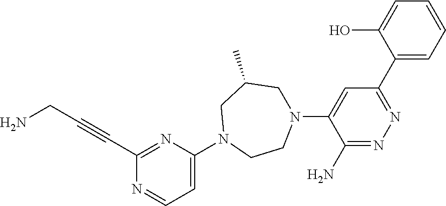 [SMILES: C[C@H]1CN(c2ccnc(C#CCN)n2)CCN(c2cc(-c3ccccc3O)nnc2N)C1]

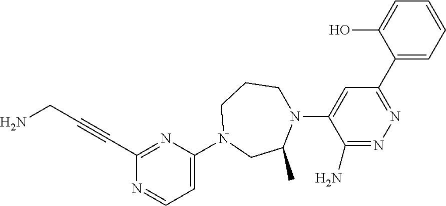 [SMILES: C[C@H]1CN(c2ccnc(C#CCN)n2)CCCN1c1cc(-c2ccccc2O)nnc1N]

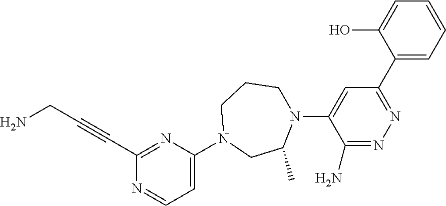 [SMILES: C[C@@H]1CN(c2ccnc(C#CCN)n2)CCCN1c1cc(-c2ccccc2O)nnc1N]

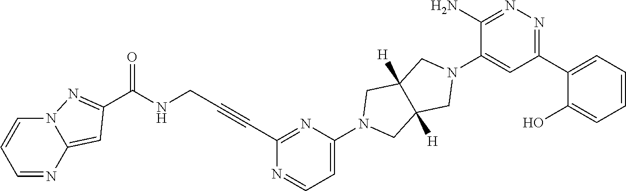 [SMILES: Nc1nnc(-c2ccccc2O)cc1N1C[C@H]2CN(c3ccnc(C#CCNC(=O)c4cc5ncccn5n4)n3)C[C@H]2C1]

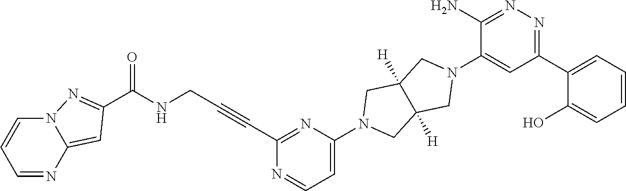 [SMILES: Nc1nnc(-c2ccccc2O)cc1N1C[C@H]2CN(c3ccnc(C#CCNC(=O)c4cc5ncccn5n4)n3)C[C@H]2C1]

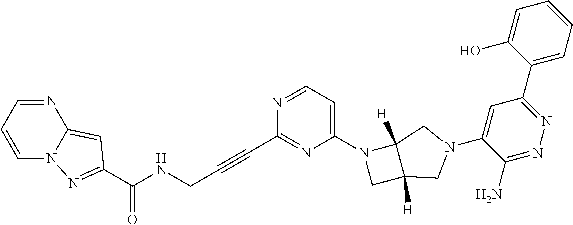 [SMILES: Nc1nnc(-c2ccccc2O)cc1N1C[C@@H]2CN(c3ccnc(C#CCNC(=O)c4cc5ncccn5n4)n3)[C@@H]2C1]

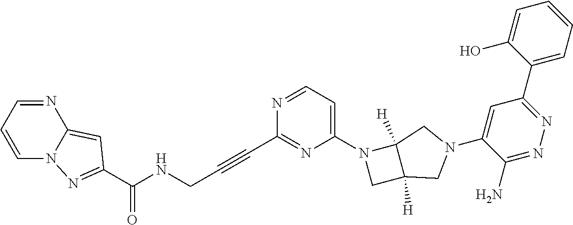 [SMILES: Nc1nnc(-c2ccccc2O)cc1N1C[C@H]2CN(c3ccnc(C#CCNC(=O)c4cc5ncccn5n4)n3)[C@H]2C1]

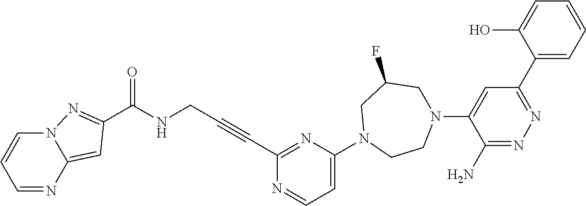 [SMILES: Nc1nnc(-c2ccccc2O)cc1N1CCN(c2ccnc(C#CCNC(=O)c3cc4ncccn4n3)n2)C[C@@H](F)C1]

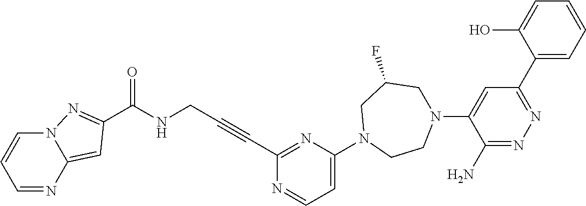 [SMILES: Nc1nnc(-c2ccccc2O)cc1N1CCN(c2ccnc(C#CCNC(=O)c3cc4ncccn4n3)n2)C[C@H](F)C1]

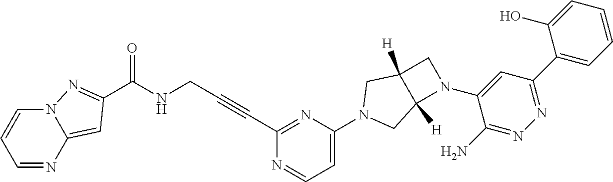 [SMILES: Nc1nnc(-c2ccccc2O)cc1N1C[C@H]2CN(c3ccnc(C#CCNC(=O)c4cc5ncccn5n4)n3)C[C@H]21]

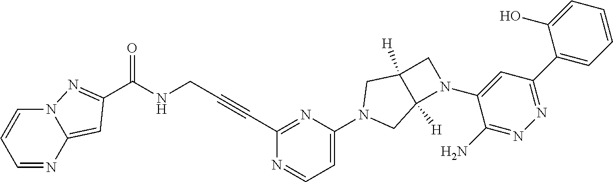 [SMILES: Nc1nnc(-c2ccccc2O)cc1N1C[C@@H]2CN(c3ccnc(C#CCNC(=O)c4cc5ncccn5n4)n3)C[C@@H]21]

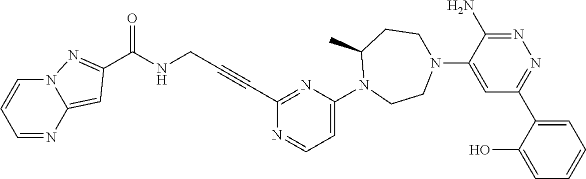

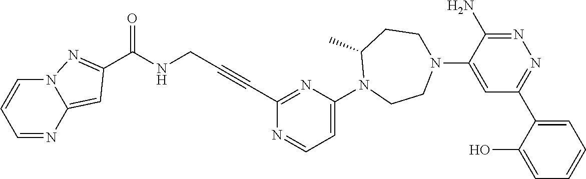

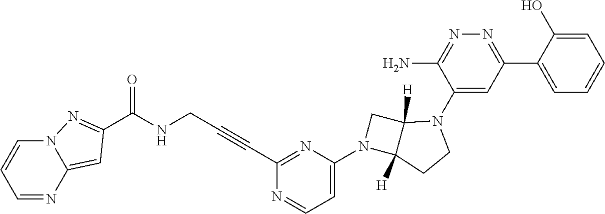 [SMILES: Nc1nnc(-c2ccccc2O)cc1N1CC[C@H]2[C@@H]1CN2c1ccnc(C#CCNC(=O)c2cc3ncccn3n2)n1]

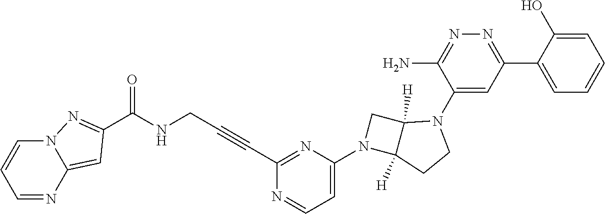 [SMILES: Nc1nnc(-c2ccccc2O)cc1N1CC[C@@H]2[C@H]1CN2c1ccnc(C#CCNC(=O)c2cc3ncccn3n2)n1]

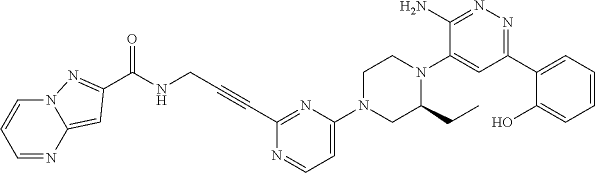 [SMILES: CC[C@H]1CN(c2ccnc(C#CCNC(=O)c3cc4ncccn4n3)n2)CCN1c1cc(-c2ccccc2O)nnc1N]

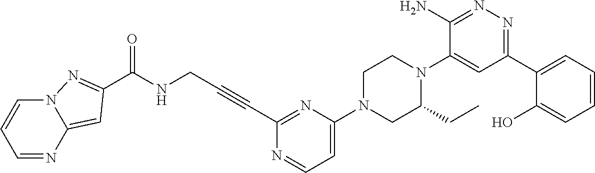 [SMILES: CC[C@@H]1CN(c2ccnc(C#CCNC(=O)c3cc4ncccn4n3)n2)CCN1c1cc(-c2ccccc2O)nnc1N]

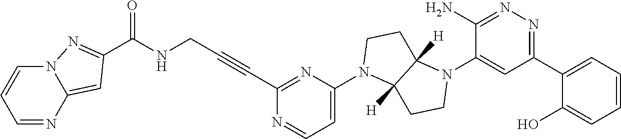 [SMILES: Nc1nnc(-c2ccccc2O)cc1N1CC[C@H]2[C@@H]1CCN2c1ccnc(C#CCNC(=O)c2cc3ncccn3n2)n1]

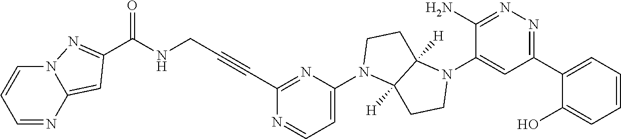 [SMILES: Nc1nnc(-c2ccccc2O)cc1N1CC[C@@H]2[C@H]1CCN2c1ccnc(C#CCNC(=O)c2cc3ncccn3n2)n1]

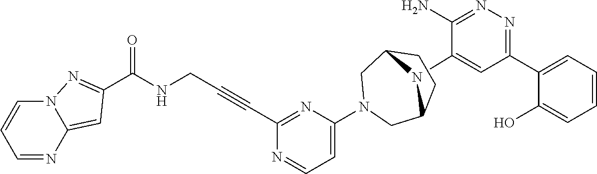

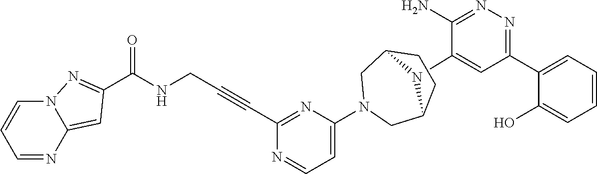 [SMILES: Nc1nnc(-c2ccccc2O)cc1N1[C@@H]2CC[C@H]1CN(c1ccnc(C#CCNC(=O)c3cc4ncccn4n3)n1)C2]

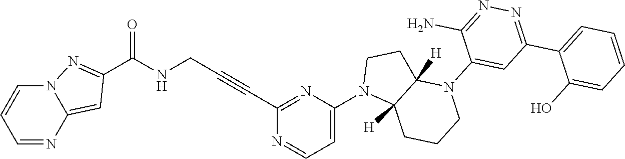 [SMILES: Nc1nnc(-c2ccccc2O)cc1N1CCC[C@H]2[C@@H]1CCN2c1ccnc(C#CCNC(=O)c2cc3ncccn3n2)n1]

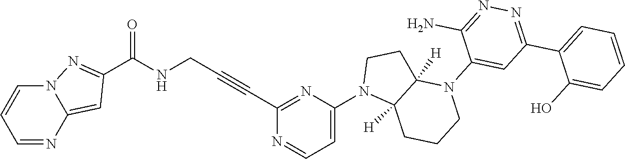 [SMILES: Nc1nnc(-c2ccccc2O)cc1N1CCC[C@@H]2[C@H]1CCN2c1ccnc(C#CCNC(=O)c2cc3ncccn3n2)n1]

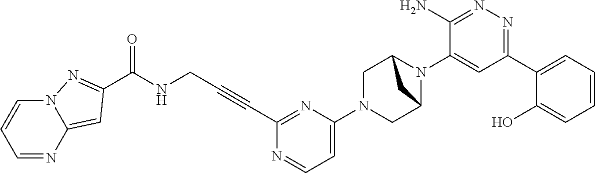 [SMILES: Nc1nnc(-c2ccccc2O)cc1N1[C@@H]2C[C@H]1CN(c1ccnc(C#CCNC(=O)c3cc4ncccn4n3)n1)C2]

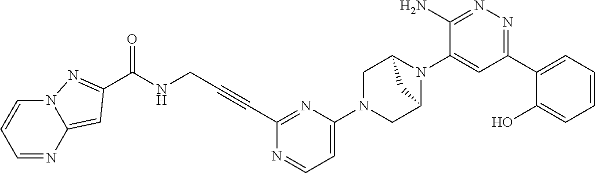 [SMILES: Nc1nnc(-c2ccccc2O)cc1N1[C@@H]2C[C@H]1CN(c1ccnc(C#CCNC(=O)c3cc4ncccn4n3)n1)C2]

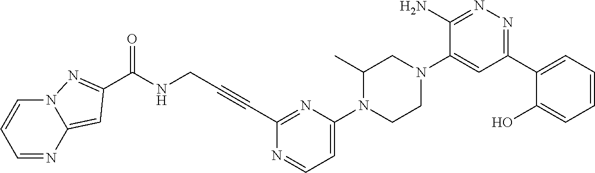 [SMILES: CC1CN(c2cc(-c3ccccc3O)nnc2N)CCN1c1ccnc(C#CCNC(=O)c2cc3ncccn3n2)n1]

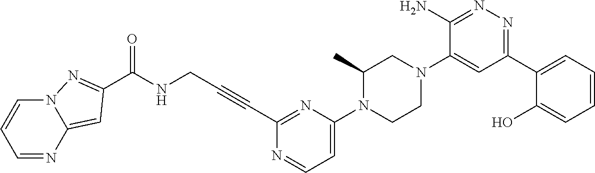 [SMILES: C[C@H]1CN(c2cc(-c3ccccc3O)nnc2N)CCN1c1ccnc(C#CCNC(=O)c2cc3ncccn3n2)n1]

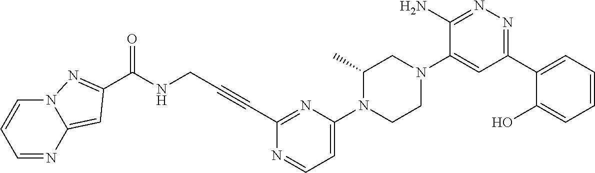 [SMILES: C[C@@H]1CN(c2cc(-c3ccccc3O)nnc2N)CCN1c1ccnc(C#CCNC(=O)c2cc3ncccn3n2)n1]

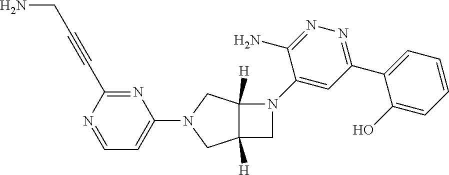 [SMILES: NCC#Cc1nccc(N2C[C@H]3CN(c4cc(-c5ccccc5O)nnc4N)[C@H]3C2)n1]

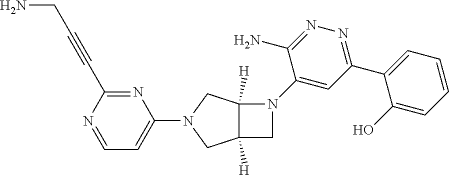 [SMILES: NCC#Cc1nccc(N2C[C@@H]3CN(c4cc(-c5ccccc5O)nnc4N)[C@@H]3C2)n1]

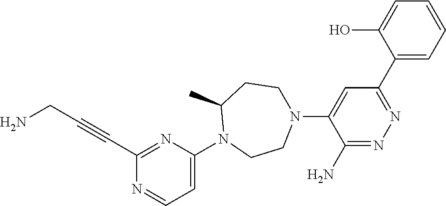 [SMILES: C[C@H]1CCN(c2cc(-c3ccccc3O)nnc2N)CCN1c1ccnc(C#CCN)n1]

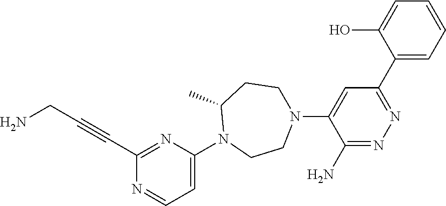 [SMILES: C[C@@H]1CCN(c2cc(-c3ccccc3O)nnc2N)CCN1c1ccnc(C#CCN)n1]

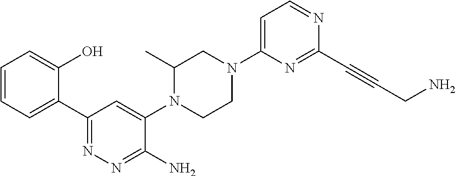 [SMILES: CC1CN(c2ccnc(C#CCN)n2)CCN1c1cc(-c2ccccc2O)nnc1N]

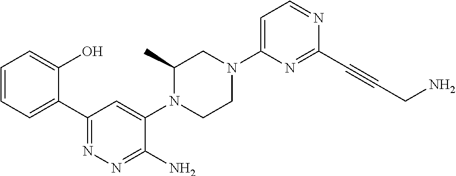 [SMILES: C[C@H]1CN(c2ccnc(C#CCN)n2)CCN1c1cc(-c2ccccc2O)nnc1N]

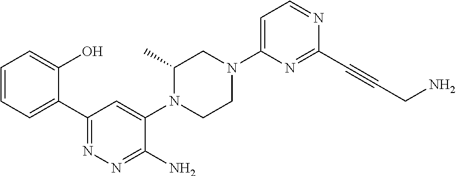 [SMILES: C[C@@H]1CN(c2ccnc(C#CCN)n2)CCN1c1cc(-c2ccccc2O)nnc1N]

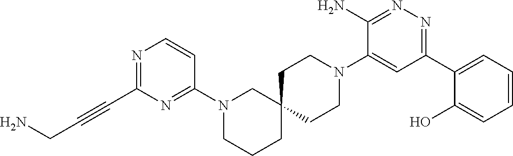

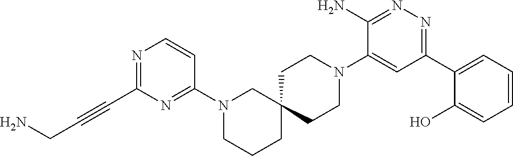

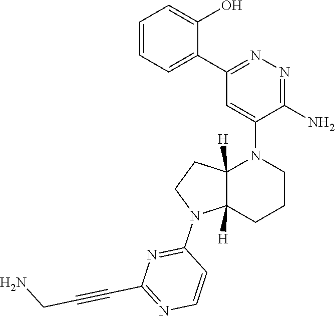 [SMILES: NCC#Cc1nccc(N2CC[C@H]3[C@@H]2CCCN3c2cc(-c3ccccc3O)nnc2N)n1]

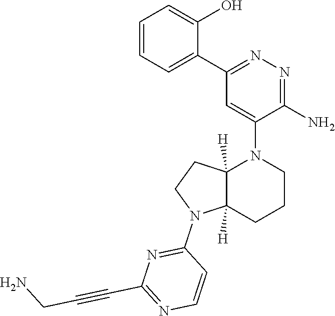 [SMILES: NCC#Cc1nccc(N2CC[C@@H]3[C@H]2CCCN3c2cc(-c3ccccc3O)nnc2N)n1]

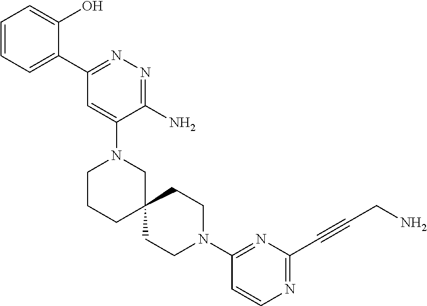

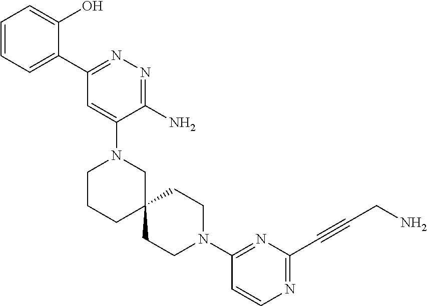

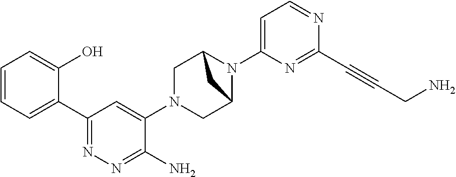 [SMILES: NCC#Cc1nccc(N2[C@@H]3C[C@H]2CN(c2cc(-c4ccccc4O)nnc2N)C3)n1]

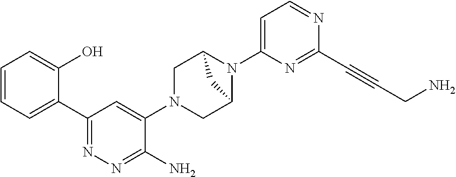 [SMILES: NCC#Cc1nccc(N2[C@@H]3C[C@H]2CN(c2cc(-c4ccccc4O)nnc2N)C3)n1]

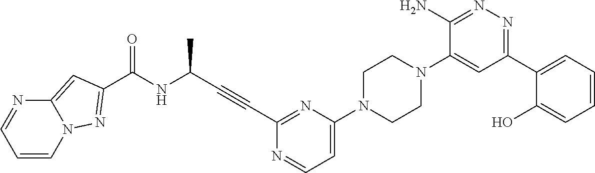 [SMILES: C[C@@H](C#Cc1nccc(N2CCN(c3cc(-c4ccccc4O)nnc3N)CC2)n1)NC(=O)c1cc2ncccn2n1]

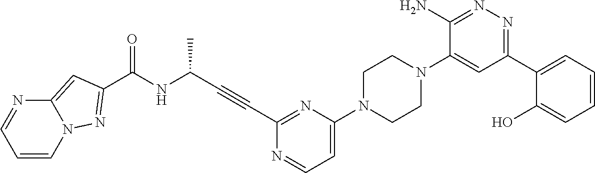 [SMILES: C[C@H](C#Cc1nccc(N2CCN(c3cc(-c4ccccc4O)nnc3N)CC2)n1)NC(=O)c1cc2ncccn2n1]

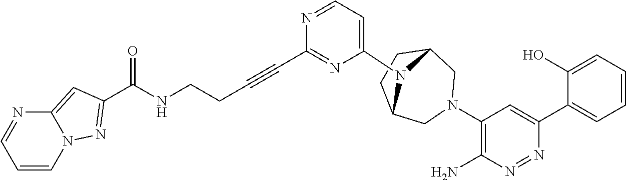 [SMILES: Nc1nnc(-c2ccccc2O)cc1N1C[C@H]2CC[C@@H](C1)N2c1ccnc(C#CCCNC(=O)c2cc3ncccn3n2)n1]

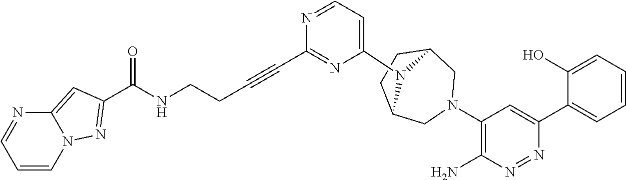 [SMILES: Nc1nnc(-c2ccccc2O)cc1N1C[C@H]2CC[C@@H](C1)N2c1ccnc(C#CCCNC(=O)c2cc3ncccn3n2)n1]

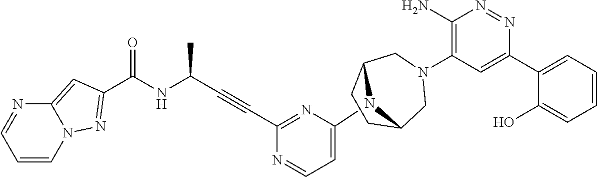 [SMILES: C[C@@H](C#Cc1nccc(N2[C@@H]3CC[C@H]2CN(c2cc(-c4ccccc4O)nnc2N)C3)n1)NC(=O)c1cc2ncccn2n1]

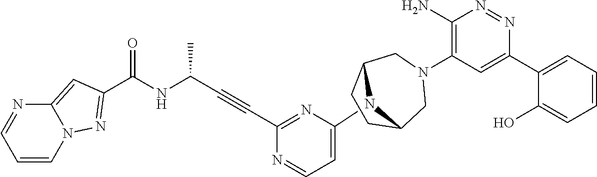 [SMILES: C[C@H](C#Cc1nccc(N2[C@@H]3CC[C@H]2CN(c2cc(-c4ccccc4O)nnc2N)C3)n1)NC(=O)c1cc2ncccn2n1]

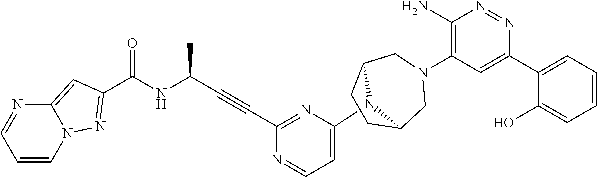 [SMILES: C[C@@H](C#Cc1nccc(N2[C@@H]3CC[C@H]2CN(c2cc(-c4ccccc4O)nnc2N)C3)n1)NC(=O)c1cc2ncccn2n1]

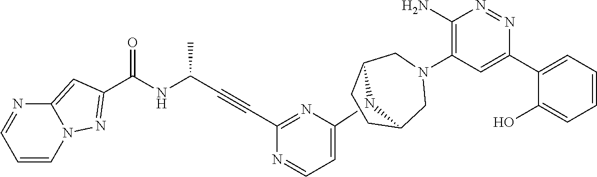 [SMILES: C[C@H](C#Cc1nccc(N2[C@@H]3CC[C@H]2CN(c2cc(-c4ccccc4O)nnc2N)C3)n1)NC(=O)c1cc2ncccn2n1]

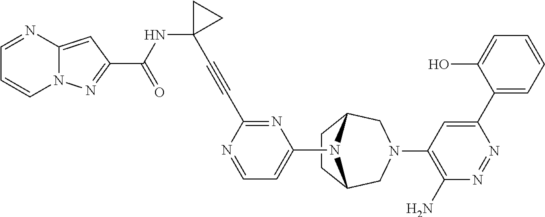 [SMILES: Nc1nnc(-c2ccccc2O)cc1N1C[C@H]2CC[C@@H](C1)N2c1ccnc(C#CC2(NC(=O)c3cc4ncccn4n3)CC2)n1]

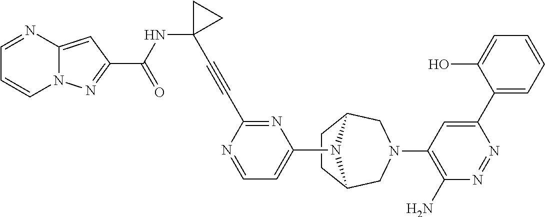 [SMILES: Nc1nnc(-c2ccccc2O)cc1N1C[C@H]2CC[C@@H](C1)N2c1ccnc(C#CC2(NC(=O)c3cc4ncccn4n3)CC2)n1]

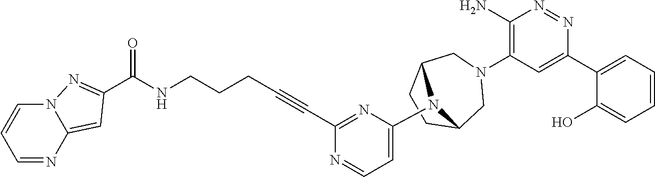 [SMILES: Nc1nnc(-c2ccccc2O)cc1N1C[C@H]2CC[C@@H](C1)N2c1ccnc(C#CCCCNC(=O)c2cc3ncccn3n2)n1]

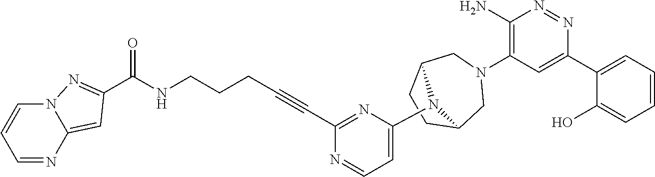 [SMILES: Nc1nnc(-c2ccccc2O)cc1N1C[C@H]2CC[C@@H](C1)N2c1ccnc(C#CCCCNC(=O)c2cc3ncccn3n2)n1]

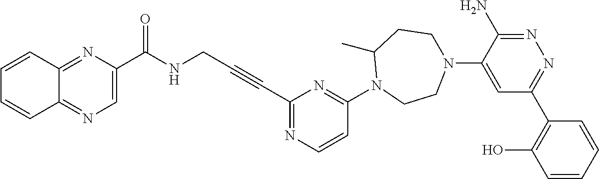 [SMILES: CC1CCN(c2cc(-c3ccccc3O)nnc2N)CCN1c1ccnc(C#CCNC(=O)c2cnc3ccccc3n2)n1]

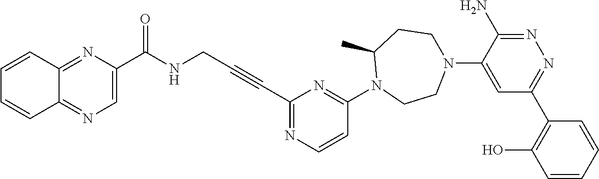 [SMILES: C[C@H]1CCN(c2cc(-c3ccccc3O)nnc2N)CCN1c1ccnc(C#CCNC(=O)c2cnc3ccccc3n2)n1]

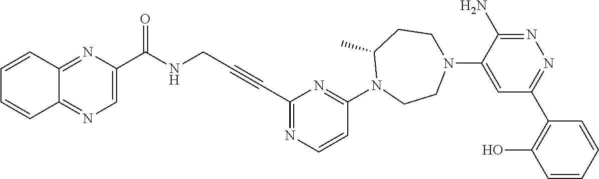 [SMILES: C[C@@H]1CCN(c2cc(-c3ccccc3O)nnc2N)CCN1c1ccnc(C#CCNC(=O)c2cnc3ccccc3n2)n1]

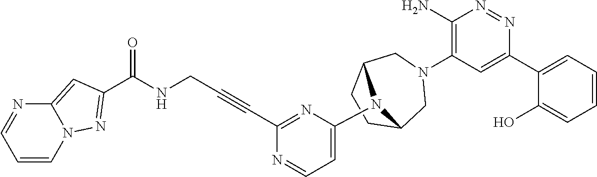 [SMILES: Nc1nnc(-c2ccccc2O)cc1N1C[C@H]2CC[C@@H](C1)N2c1ccnc(C#CCNC(=O)c2cc3ncccn3n2)n1]

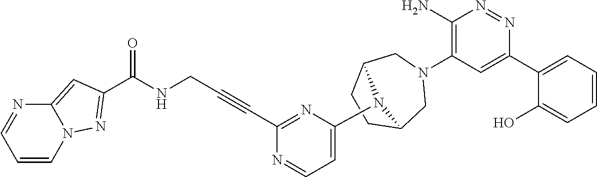 [SMILES: Nc1nnc(-c2ccccc2O)cc1N1C[C@H]2CC[C@@H](C1)N2c1ccnc(C#CCNC(=O)c2cc3ncccn3n2)n1]

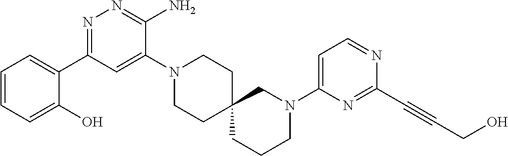

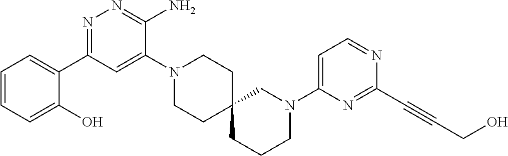

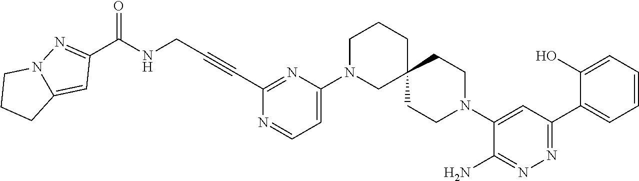

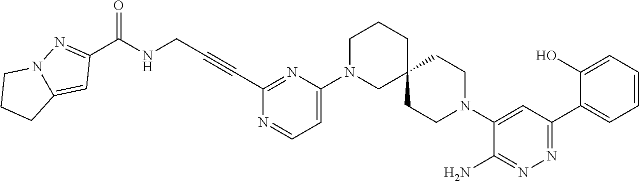

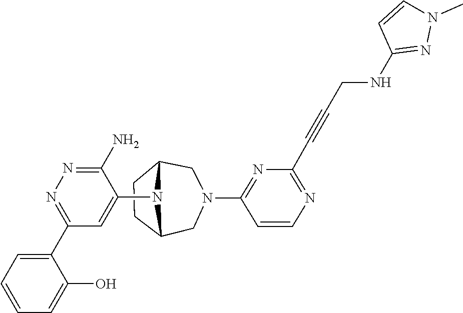 [SMILES: Cn1ccc(NCC#Cc2nccc(N3C[C@H]4CC[C@@H](C3)N4c3cc(-c4ccccc4O)nnc3N)n2)n1]

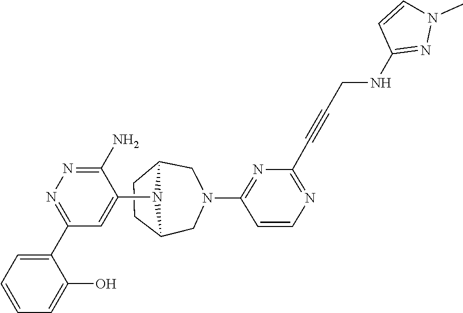 [SMILES: Cn1ccc(NCC#Cc2nccc(N3C[C@H]4CC[C@@H](C3)N4c3cc(-c4ccccc4O)nnc3N)n2)n1]

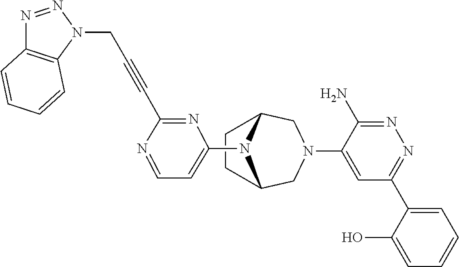 [SMILES: Nc1nnc(-c2ccccc2O)cc1N1C[C@H]2CC[C@@H](C1)N2c1ccnc(C#CCn2nnc3ccccc32)n1]

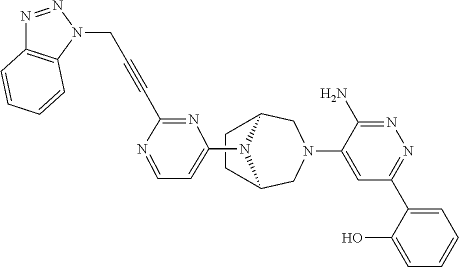 [SMILES: Nc1nnc(-c2ccccc2O)cc1N1C[C@H]2CC[C@@H](C1)N2c1ccnc(C#CCn2nnc3ccccc32)n1]

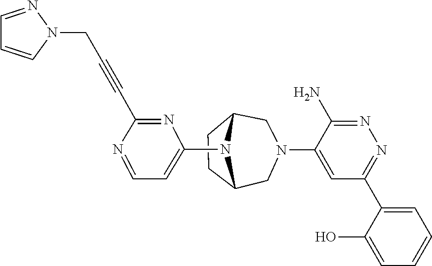 [SMILES: Nc1nnc(-c2ccccc2O)cc1N1C[C@H]2CC[C@@H](C1)N2c1ccnc(C#CCn2cccn2)n1]

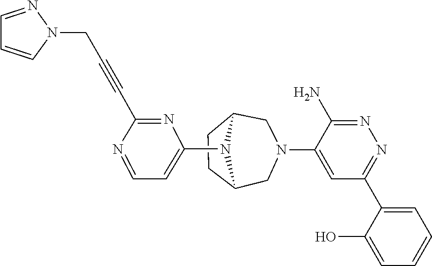 [SMILES: Nc1nnc(-c2ccccc2O)cc1N1C[C@H]2CC[C@@H](C1)N2c1ccnc(C#CCn2cccn2)n1]

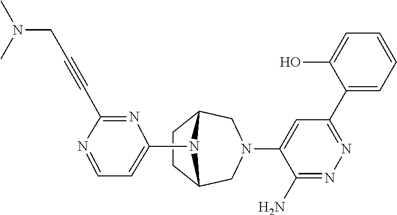 [SMILES: CN(C)CC#Cc1nccc(N2[C@@H]3CC[C@H]2CN(c2cc(-c4ccccc4O)nnc2N)C3)n1]

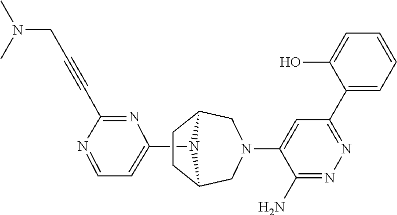 [SMILES: CN(C)CC#Cc1nccc(N2[C@@H]3CC[C@H]2CN(c2cc(-c4ccccc4O)nnc2N)C3)n1]

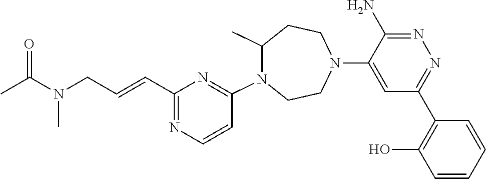 [SMILES: CC(=O)N(C)C/C=C/c1nccc(N2CCN(c3cc(-c4ccccc4O)nnc3N)CCC2C)n1]

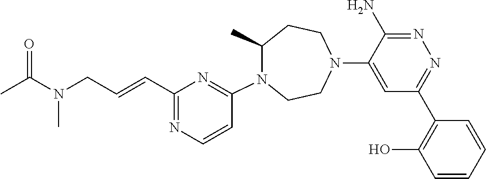 [SMILES: CC(=O)N(C)C/C=C/c1nccc(N2CCN(c3cc(-c4ccccc4O)nnc3N)CC[C@@H]2C)n1]

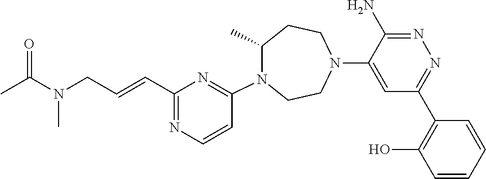 [SMILES: CC(=O)N(C)C/C=C/c1nccc(N2CCN(c3cc(-c4ccccc4O)nnc3N)CC[C@H]2C)n1]

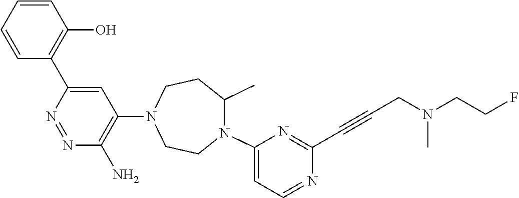 [SMILES: CC1CCN(c2cc(-c3ccccc3O)nnc2N)CCN1c1ccnc(C#CCN(C)CCF)n1]

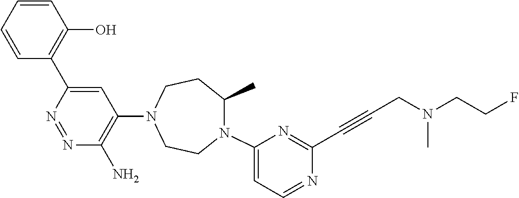 [SMILES: C[C@@H]1CCN(c2cc(-c3ccccc3O)nnc2N)CCN1c1ccnc(C#CCN(C)CCF)n1]

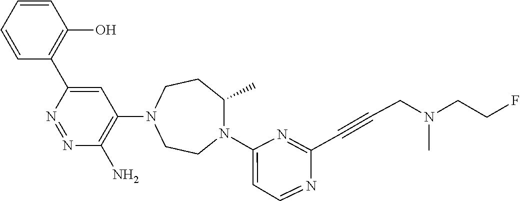 [SMILES: C[C@H]1CCN(c2cc(-c3ccccc3O)nnc2N)CCN1c1ccnc(C#CCN(C)CCF)n1]

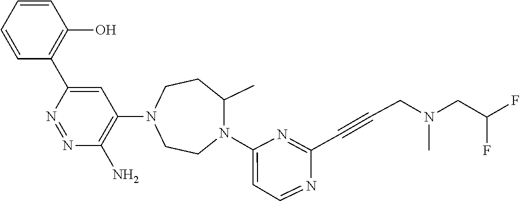 [SMILES: CC1CCN(c2cc(-c3ccccc3O)nnc2N)CCN1c1ccnc(C#CCN(C)CC(F)F)n1]

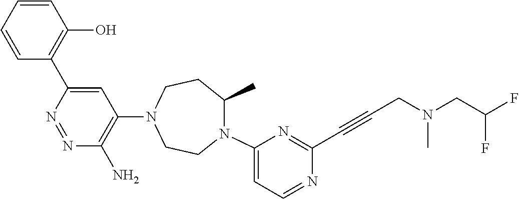 [SMILES: C[C@@H]1CCN(c2cc(-c3ccccc3O)nnc2N)CCN1c1ccnc(C#CCN(C)CC(F)F)n1]

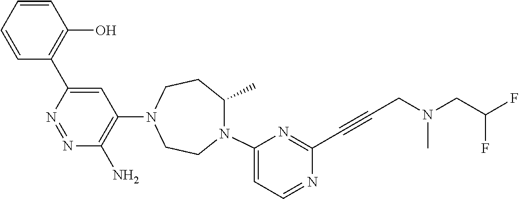 [SMILES: C[C@H]1CCN(c2cc(-c3ccccc3O)nnc2N)CCN1c1ccnc(C#CCN(C)CC(F)F)n1]

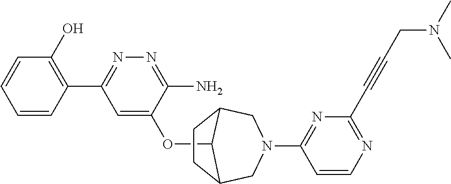 [SMILES: CN(C)CC#Cc1nccc(N2CC3CCC(C2)C3Oc2cc(-c3ccccc3O)nnc2N)n1]

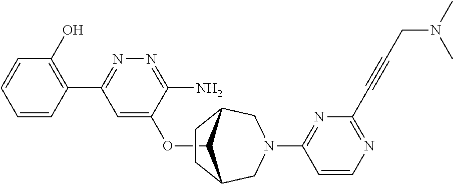 [SMILES: CN(C)CC#Cc1nccc(N2C[C@H]3CC[C@@H](C2)C3Oc2cc(-c3ccccc3O)nnc2N)n1]

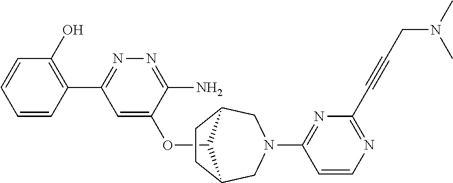 [SMILES: CN(C)CC#Cc1nccc(N2C[C@H]3CC[C@@H](C2)C3Oc2cc(-c3ccccc3O)nnc2N)n1]

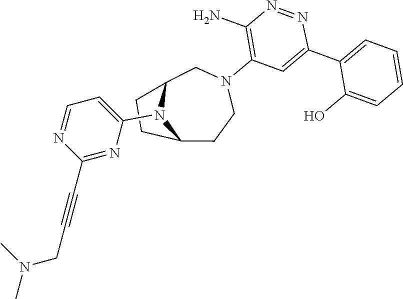 [SMILES: CN(C)CC#Cc1nccc(N2[C@H]3CC[C@@H]2CN(c2cc(-c4ccccc4O)nnc2N)CC3)n1]

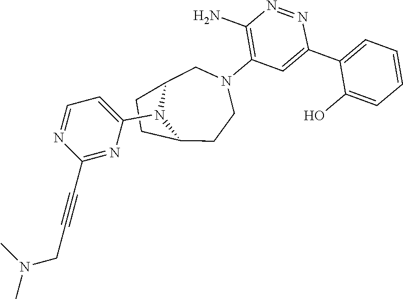 [SMILES: CN(C)CC#Cc1nccc(N2[C@@H]3CC[C@H]2CN(c2cc(-c4ccccc4O)nnc2N)CC3)n1]

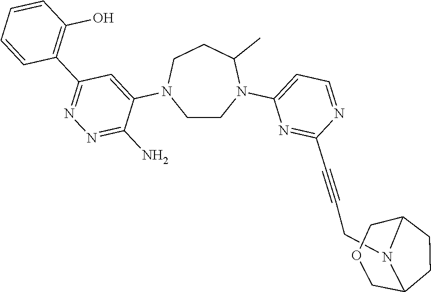 [SMILES: CC1CCN(c2cc(-c3ccccc3O)nnc2N)CCN1c1ccnc(C#CCN2C3CCC2COC3)n1]

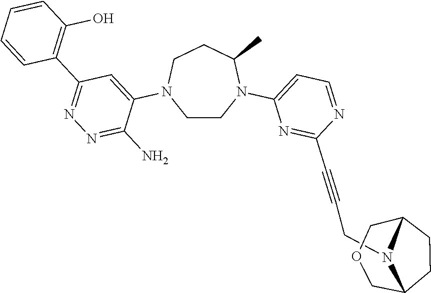 [SMILES: C[C@@H]1CCN(c2cc(-c3ccccc3O)nnc2N)CCN1c1ccnc(C#CCN2[C@@H]3CC[C@H]2COC3)n1]

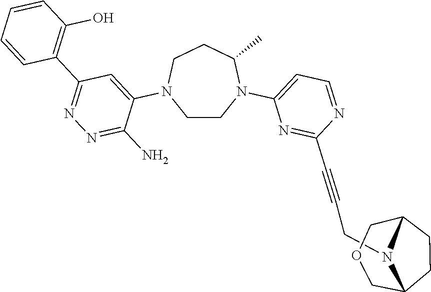 [SMILES: C[C@H]1CCN(c2cc(-c3ccccc3O)nnc2N)CCN1c1ccnc(C#CCN2[C@@H]3CC[C@H]2COC3)n1]

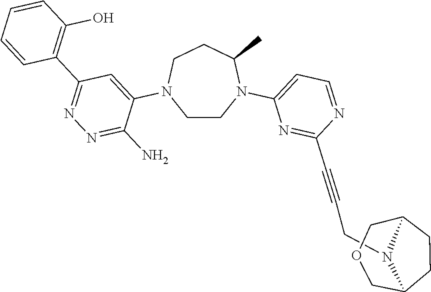 [SMILES: C[C@@H]1CCN(c2cc(-c3ccccc3O)nnc2N)CCN1c1ccnc(C#CCN2[C@@H]3CC[C@H]2COC3)n1]

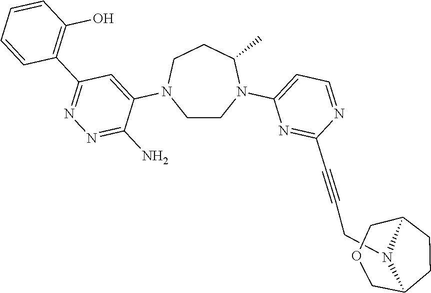 [SMILES: C[C@H]1CCN(c2cc(-c3ccccc3O)nnc2N)CCN1c1ccnc(C#CCN2[C@@H]3CC[C@H]2COC3)n1]

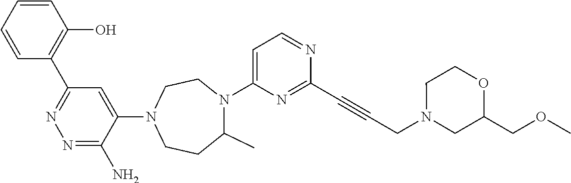 [SMILES: COCC1CN(CC#Cc2nccc(N3CCN(c4cc(-c5ccccc5O)nnc4N)CCC3C)n2)CCO1]

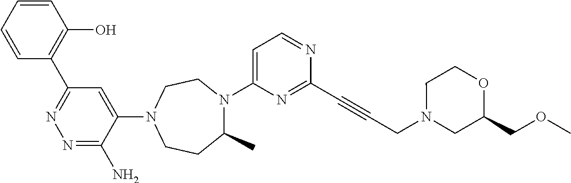 [SMILES: COC[C@H]1CN(CC#Cc2nccc(N3CCN(c4cc(-c5ccccc5O)nnc4N)CC[C@@H]3C)n2)CCO1]

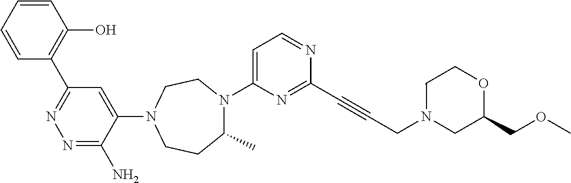 [SMILES: COC[C@H]1CN(CC#Cc2nccc(N3CCN(c4cc(-c5ccccc5O)nnc4N)CC[C@H]3C)n2)CCO1]

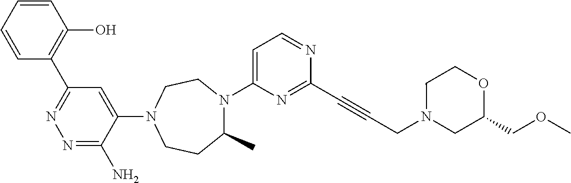 [SMILES: COC[C@@H]1CN(CC#Cc2nccc(N3CCN(c4cc(-c5ccccc5O)nnc4N)CC[C@@H]3C)n2)CCO1]

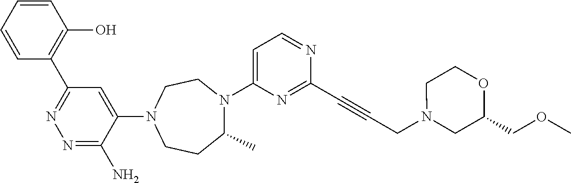 [SMILES: COC[C@@H]1CN(CC#Cc2nccc(N3CCN(c4cc(-c5ccccc5O)nnc4N)CC[C@H]3C)n2)CCO1]

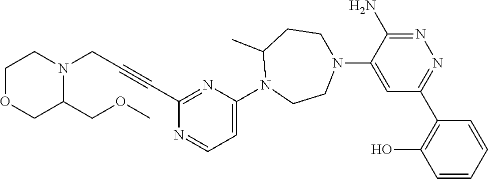 [SMILES: COCC1COCCN1CC#Cc1nccc(N2CCN(c3cc(-c4ccccc4O)nnc3N)CCC2C)n1]

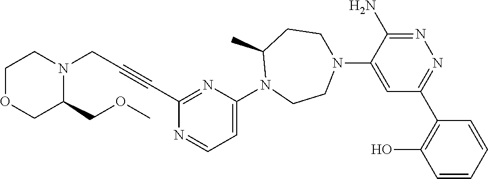 [SMILES: COC[C@H]1COCCN1CC#Cc1nccc(N2CCN(c3cc(-c4ccccc4O)nnc3N)CC[C@@H]2C)n1]

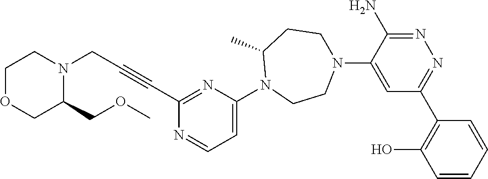 [SMILES: COC[C@H]1COCCN1CC#Cc1nccc(N2CCN(c3cc(-c4ccccc4O)nnc3N)CC[C@H]2C)n1]

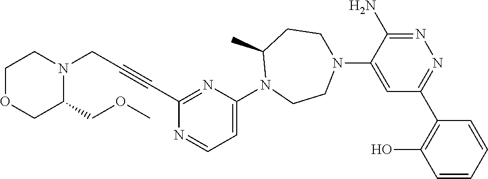 [SMILES: COC[C@@H]1COCCN1CC#Cc1nccc(N2CCN(c3cc(-c4ccccc4O)nnc3N)CC[C@@H]2C)n1]

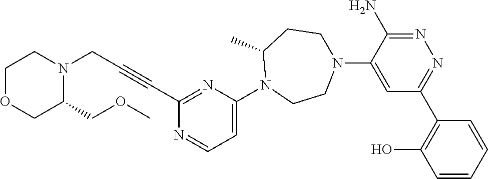 [SMILES: COC[C@@H]1COCCN1CC#Cc1nccc(N2CCN(c3cc(-c4ccccc4O)nnc3N)CC[C@H]2C)n1]

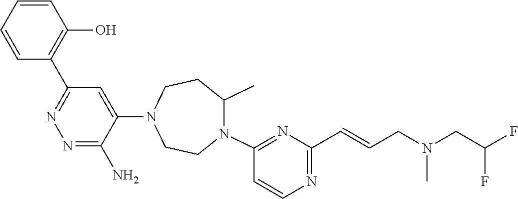 [SMILES: CC1CCN(c2cc(-c3ccccc3O)nnc2N)CCN1c1ccnc(/C=C/CN(C)CC(F)F)n1]

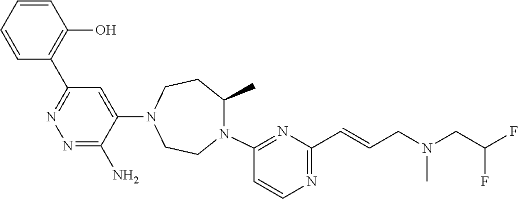 [SMILES: C[C@@H]1CCN(c2cc(-c3ccccc3O)nnc2N)CCN1c1ccnc(/C=C/CN(C)CC(F)F)n1]

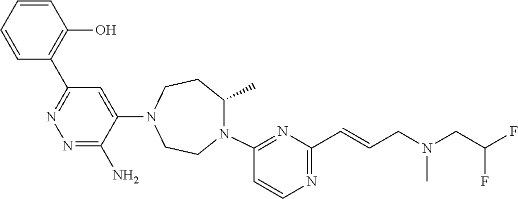 [SMILES: C[C@H]1CCN(c2cc(-c3ccccc3O)nnc2N)CCN1c1ccnc(/C=C/CN(C)CC(F)F)n1]

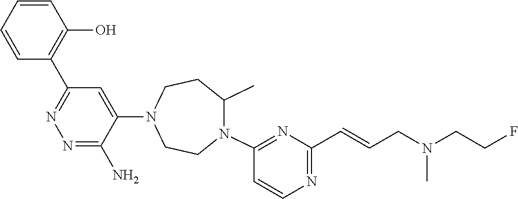 [SMILES: CC1CCN(c2cc(-c3ccccc3O)nnc2N)CCN1c1ccnc(/C=C/CN(C)CCF)n1]

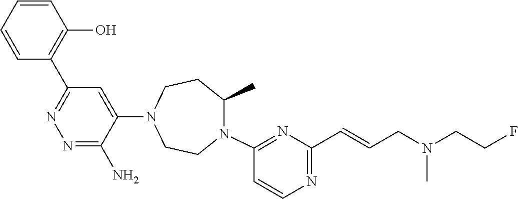 [SMILES: C[C@@H]1CCN(c2cc(-c3ccccc3O)nnc2N)CCN1c1ccnc(/C=C/CN(C)CCF)n1]

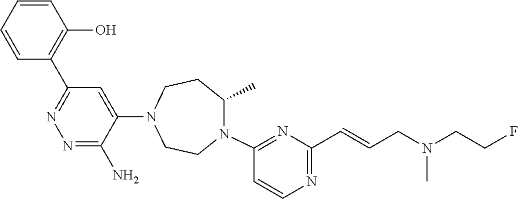 [SMILES: C[C@H]1CCN(c2cc(-c3ccccc3O)nnc2N)CCN1c1ccnc(/C=C/CN(C)CCF)n1]

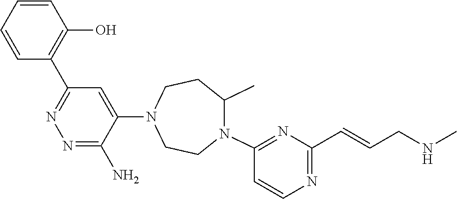 [SMILES: CNC/C=C/c1nccc(N2CCN(c3cc(-c4ccccc4O)nnc3N)CCC2C)n1]

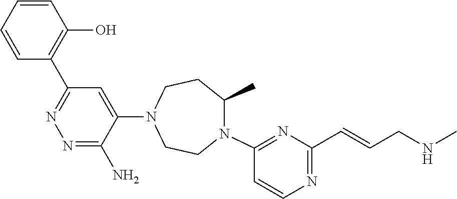 [SMILES: CNC/C=C/c1nccc(N2CCN(c3cc(-c4ccccc4O)nnc3N)CC[C@H]2C)n1]

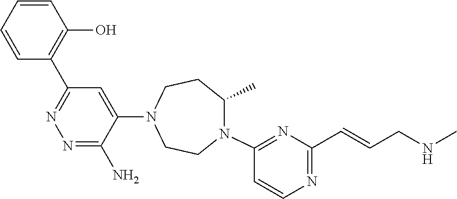 [SMILES: CNC/C=C/c1nccc(N2CCN(c3cc(-c4ccccc4O)nnc3N)CC[C@@H]2C)n1]

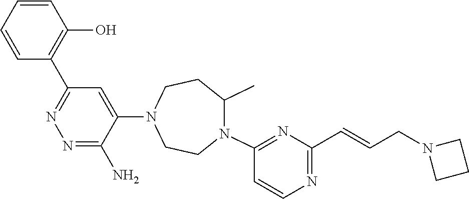 [SMILES: CC1CCN(c2cc(-c3ccccc3O)nnc2N)CCN1c1ccnc(/C=C/CN2CCC2)n1]

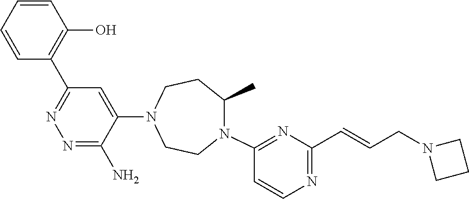 [SMILES: C[C@@H]1CCN(c2cc(-c3ccccc3O)nnc2N)CCN1c1ccnc(/C=C/CN2CCC2)n1]

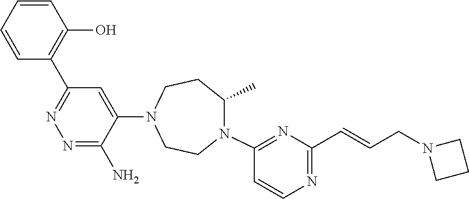 [SMILES: C[C@H]1CCN(c2cc(-c3ccccc3O)nnc2N)CCN1c1ccnc(/C=C/CN2CCC2)n1]

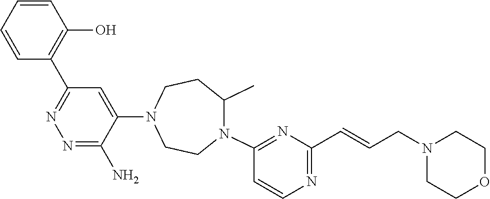 [SMILES: CC1CCN(c2cc(-c3ccccc3O)nnc2N)CCN1c1ccnc(/C=C/CN2CCOCC2)n1]

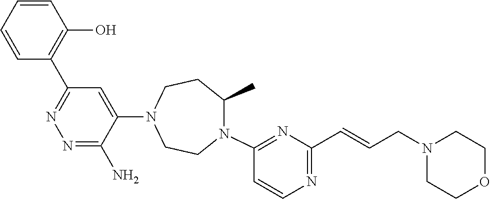 [SMILES: C[C@@H]1CCN(c2cc(-c3ccccc3O)nnc2N)CCN1c1ccnc(/C=C/CN2CCOCC2)n1]

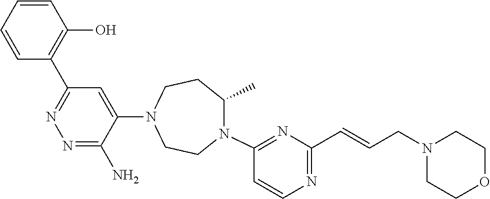 [SMILES: C[C@H]1CCN(c2cc(-c3ccccc3O)nnc2N)CCN1c1ccnc(/C=C/CN2CCOCC2)n1]

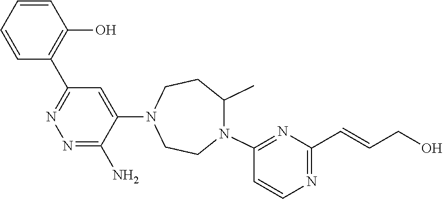 [SMILES: CC1CCN(c2cc(-c3ccccc3O)nnc2N)CCN1c1ccnc(/C=C/CO)n1]

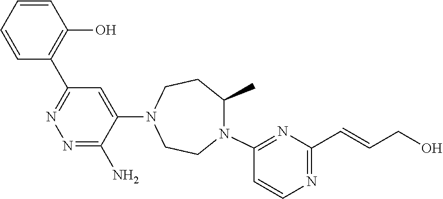 [SMILES: C[C@@H]1CCN(c2cc(-c3ccccc3O)nnc2N)CCN1c1ccnc(/C=C/CO)n1]

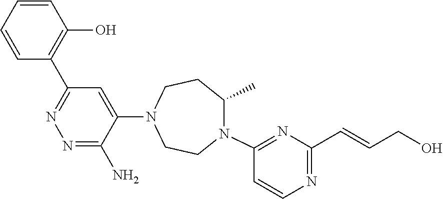 [SMILES: C[C@H]1CCN(c2cc(-c3ccccc3O)nnc2N)CCN1c1ccnc(/C=C/CO)n1]

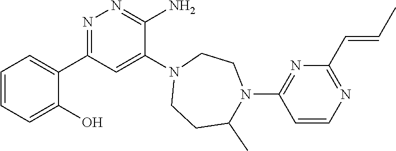 [SMILES: C/C=C/c1nccc(N2CCN(c3cc(-c4ccccc4O)nnc3N)CCC2C)n1]

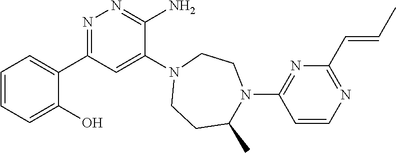 [SMILES: C/C=C/c1nccc(N2CCN(c3cc(-c4ccccc4O)nnc3N)CC[C@@H]2C)n1]

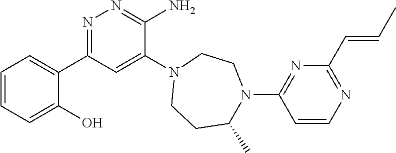 [SMILES: C/C=C/c1nccc(N2CCN(c3cc(-c4ccccc4O)nnc3N)CC[C@H]2C)n1]

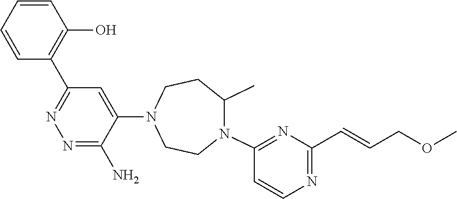 [SMILES: COC/C=C/c1nccc(N2CCN(c3cc(-c4ccccc4O)nnc3N)CCC2C)n1]

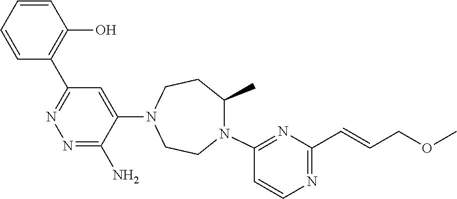 [SMILES: COC/C=C/c1nccc(N2CCN(c3cc(-c4ccccc4O)nnc3N)CC[C@H]2C)n1]

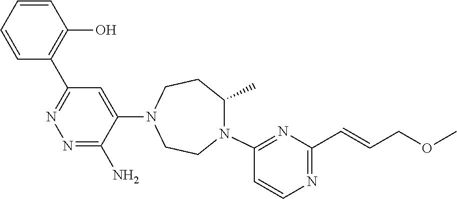 [SMILES: COC/C=C/c1nccc(N2CCN(c3cc(-c4ccccc4O)nnc3N)CC[C@@H]2C)n1]

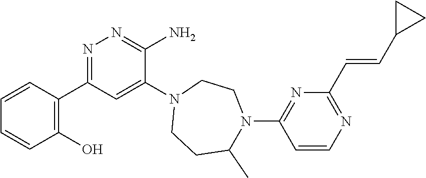 [SMILES: CC1CCN(c2cc(-c3ccccc3O)nnc2N)CCN1c1ccnc(/C=C/C2CC2)n1]

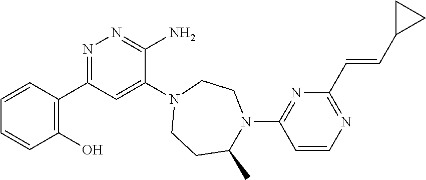 [SMILES: C[C@H]1CCN(c2cc(-c3ccccc3O)nnc2N)CCN1c1ccnc(/C=C/C2CC2)n1]

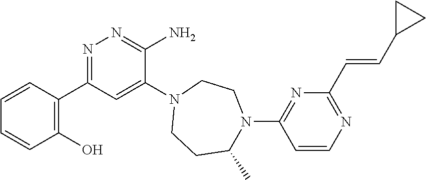 [SMILES: C[C@@H]1CCN(c2cc(-c3ccccc3O)nnc2N)CCN1c1ccnc(/C=C/C2CC2)n1]

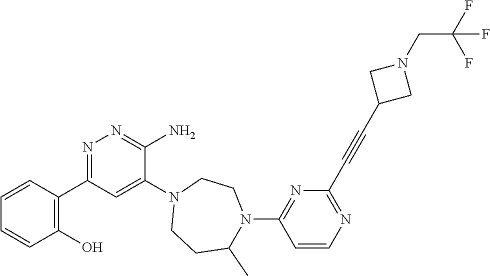 [SMILES: CC1CCN(c2cc(-c3ccccc3O)nnc2N)CCN1c1ccnc(C#CC2CN(CC(F)(F)F)C2)n1]

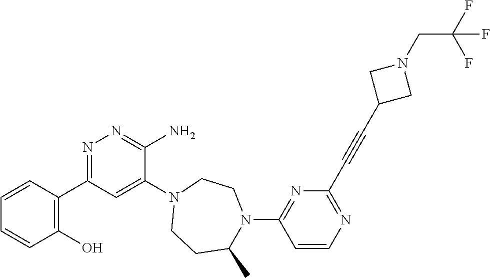 [SMILES: C[C@H]1CCN(c2cc(-c3ccccc3O)nnc2N)CCN1c1ccnc(C#CC2CN(CC(F)(F)F)C2)n1]

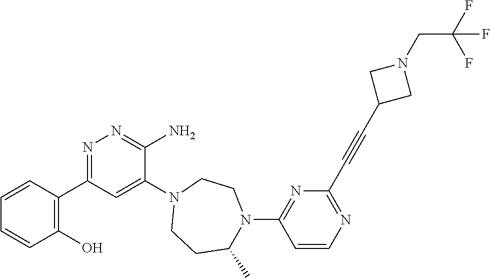 [SMILES: C[C@@H]1CCN(c2cc(-c3ccccc3O)nnc2N)CCN1c1ccnc(C#CC2CN(CC(F)(F)F)C2)n1]

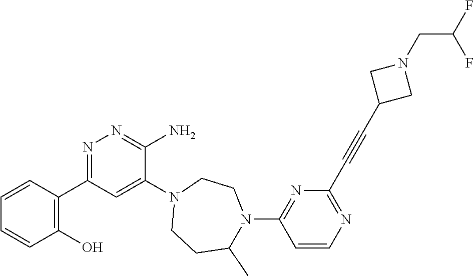 [SMILES: CC1CCN(c2cc(-c3ccccc3O)nnc2N)CCN1c1ccnc(C#CC2CN(CC(F)F)C2)n1]

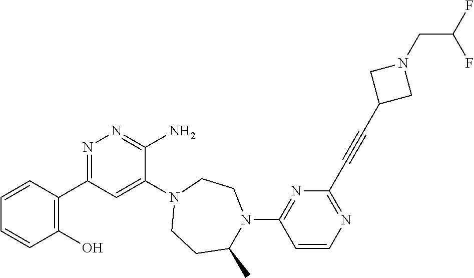 [SMILES: C[C@H]1CCN(c2cc(-c3ccccc3O)nnc2N)CCN1c1ccnc(C#CC2CN(CC(F)F)C2)n1]

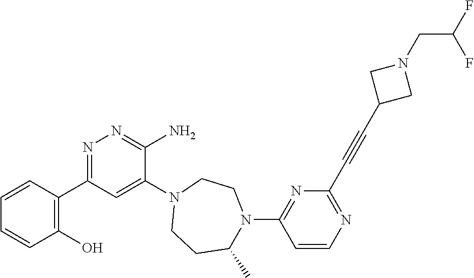 [SMILES: C[C@@H]1CCN(c2cc(-c3ccccc3O)nnc2N)CCN1c1ccnc(C#CC2CN(CC(F)F)C2)n1]

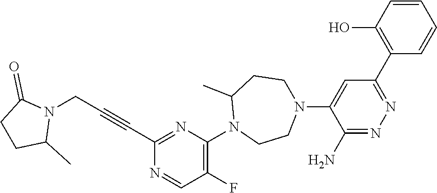 [SMILES: CC1CCC(=O)N1CC#Cc1ncc(F)c(N2CCN(c3cc(-c4ccccc4O)nnc3N)CCC2C)n1]

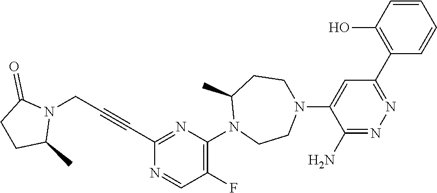 [SMILES: C[C@H]1CCC(=O)N1CC#Cc1ncc(F)c(N2CCN(c3cc(-c4ccccc4O)nnc3N)CC[C@@H]2C)n1]

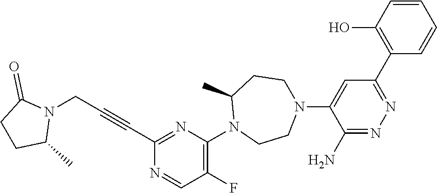 [SMILES: C[C@@H]1CCC(=O)N1CC#Cc1ncc(F)c(N2CCN(c3cc(-c4ccccc4O)nnc3N)CC[C@@H]2C)n1]

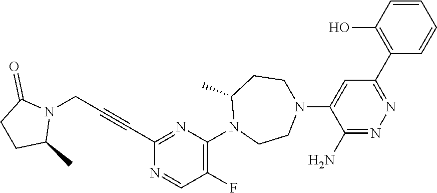 [SMILES: C[C@@H]1CCN(c2cc(-c3ccccc3O)nnc2N)CCN1c1nc(C#CCN2C(=O)CC[C@@H]2C)ncc1F]

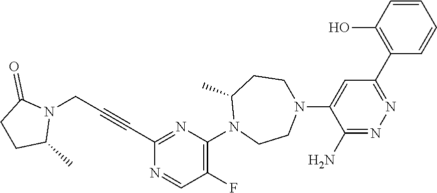 [SMILES: C[C@@H]1CCC(=O)N1CC#Cc1ncc(F)c(N2CCN(c3cc(-c4ccccc4O)nnc3N)CC[C@H]2C)n1]

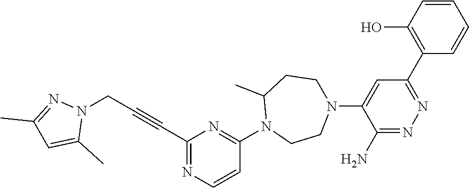 [SMILES: Cc1cc(C)n(CC#Cc2nccc(N3CCN(c4cc(-c5ccccc5O)nnc4N)CCC3C)n2)n1]

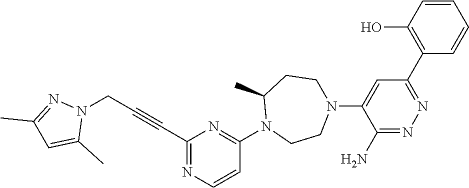 [SMILES: Cc1cc(C)n(CC#Cc2nccc(N3CCN(c4cc(-c5ccccc5O)nnc4N)CC[C@@H]3C)n2)n1]

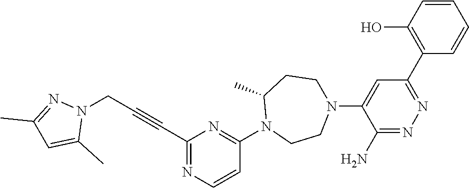 [SMILES: Cc1cc(C)n(CC#Cc2nccc(N3CCN(c4cc(-c5ccccc5O)nnc4N)CC[C@H]3C)n2)n1]

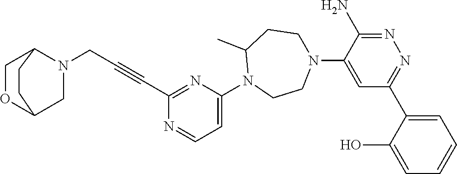 [SMILES: CC1CCN(c2cc(-c3ccccc3O)nnc2N)CCN1c1ccnc(C#CCN2CC3CCC2CO3)n1]

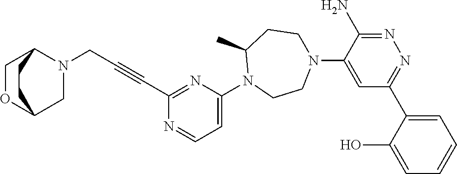 [SMILES: C[C@H]1CCN(c2cc(-c3ccccc3O)nnc2N)CCN1c1ccnc(C#CCN2C[C@H]3CC[C@@H]2CO3)n1]

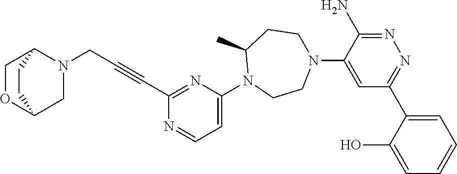 [SMILES: C[C@H]1CCN(c2cc(-c3ccccc3O)nnc2N)CCN1c1ccnc(C#CCN2C[C@@H]3CC[C@H]2CO3)n1]

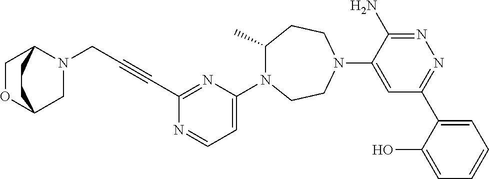 [SMILES: C[C@@H]1CCN(c2cc(-c3ccccc3O)nnc2N)CCN1c1ccnc(C#CCN2C[C@H]3CC[C@@H]2CO3)n1]

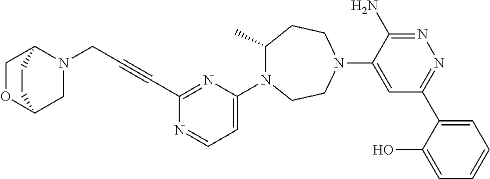 [SMILES: C[C@@H]1CCN(c2cc(-c3ccccc3O)nnc2N)CCN1c1ccnc(C#CCN2C[C@@H]3CC[C@H]2CO3)n1]

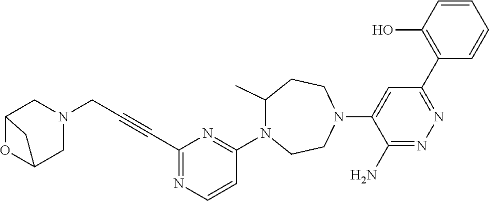 [SMILES: CC1CCN(c2cc(-c3ccccc3O)nnc2N)CCN1c1ccnc(C#CCN2CC3CC(C2)O3)n1]

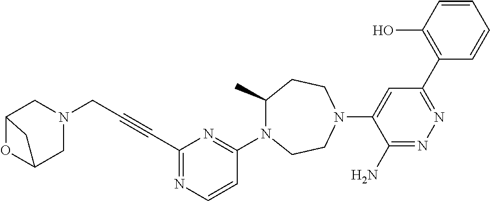 [SMILES: C[C@H]1CCN(c2cc(-c3ccccc3O)nnc2N)CCN1c1ccnc(C#CCN2CC3CC(C2)O3)n1]

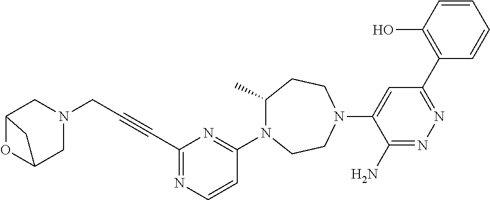 [SMILES: C[C@@H]1CCN(c2cc(-c3ccccc3O)nnc2N)CCN1c1ccnc(C#CCN2CC3CC(C2)O3)n1]

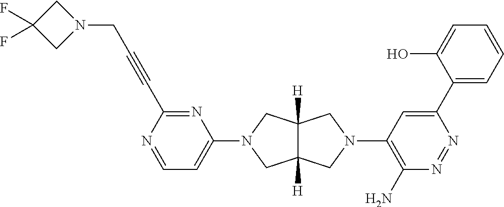 [SMILES: Nc1nnc(-c2ccccc2O)cc1N1C[C@H]2CN(c3ccnc(C#CCN4CC(F)(F)C4)n3)C[C@H]2C1]

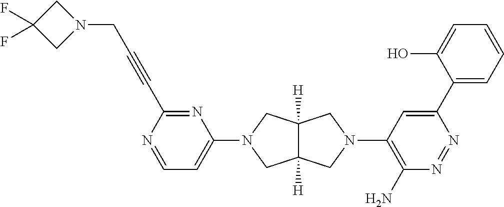 [SMILES: Nc1nnc(-c2ccccc2O)cc1N1C[C@H]2CN(c3ccnc(C#CCN4CC(F)(F)C4)n3)C[C@H]2C1]

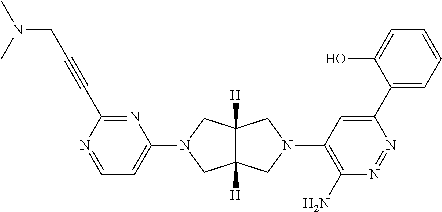 [SMILES: CN(C)CC#Cc1nccc(N2C[C@@H]3CN(c4cc(-c5ccccc5O)nnc4N)C[C@@H]3C2)n1]

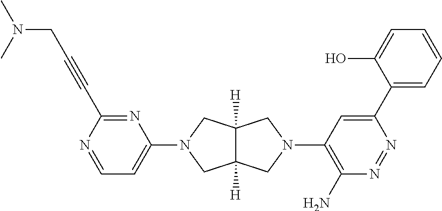 [SMILES: CN(C)CC#Cc1nccc(N2C[C@@H]3CN(c4cc(-c5ccccc5O)nnc4N)C[C@@H]3C2)n1]

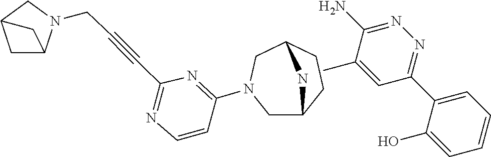

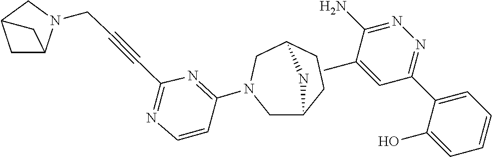

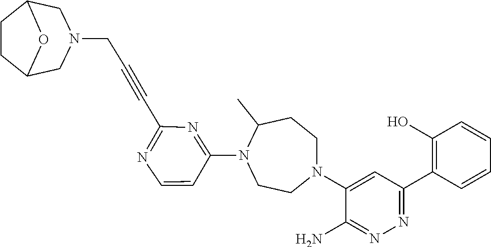 [SMILES: CC1CCN(c2cc(-c3ccccc3O)nnc2N)CCN1c1ccnc(C#CCN2CC3CCC(C2)O3)n1]

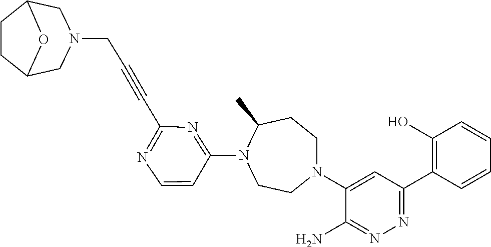 [SMILES: C[C@H]1CCN(c2cc(-c3ccccc3O)nnc2N)CCN1c1ccnc(C#CCN2CC3CCC(C2)O3)n1]

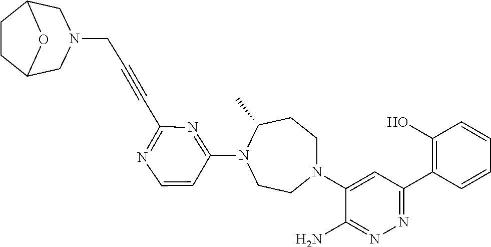 [SMILES: C[C@@H]1CCN(c2cc(-c3ccccc3O)nnc2N)CCN1c1ccnc(C#CCN2CC3CCC(C2)O3)n1]

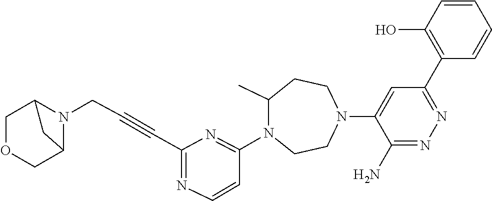 [SMILES: CC1CCN(c2cc(-c3ccccc3O)nnc2N)CCN1c1ccnc(C#CCN2C3COCC2C3)n1]

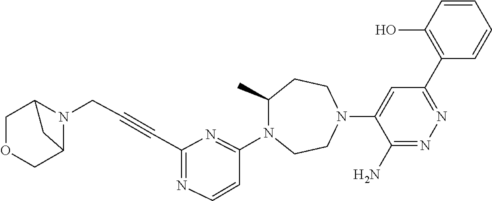 [SMILES: C[C@H]1CCN(c2cc(-c3ccccc3O)nnc2N)CCN1c1ccnc(C#CCN2C3COCC2C3)n1]

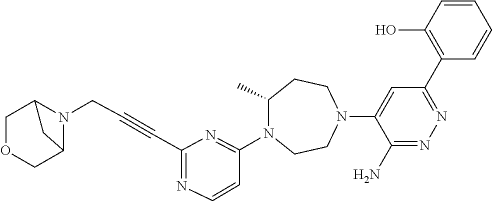 [SMILES: C[C@@H]1CCN(c2cc(-c3ccccc3O)nnc2N)CCN1c1ccnc(C#CCN2C3COCC2C3)n1]

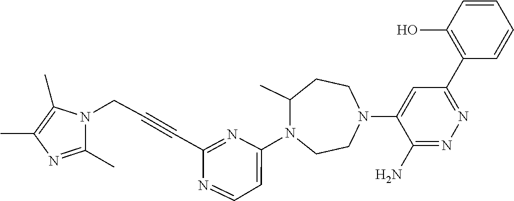 [SMILES: Cc1nc(C)n(CC#Cc2nccc(N3CCN(c4cc(-c5ccccc5O)nnc4N)CCC3C)n2)c1C]

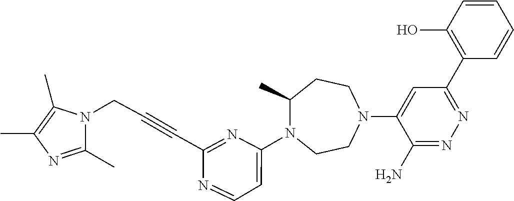 [SMILES: Cc1nc(C)n(CC#Cc2nccc(N3CCN(c4cc(-c5ccccc5O)nnc4N)CC[C@@H]3C)n2)c1C]

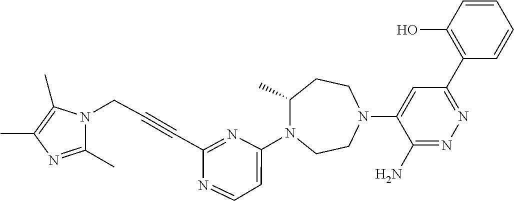 [SMILES: Cc1nc(C)n(CC#Cc2nccc(N3CCN(c4cc(-c5ccccc5O)nnc4N)CC[C@H]3C)n2)c1C]

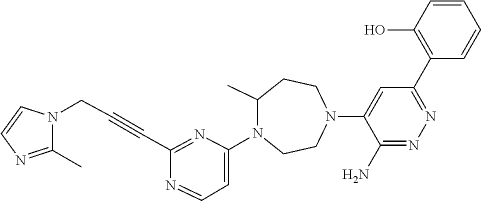 [SMILES: Cc1nccn1CC#Cc1nccc(N2CCN(c3cc(-c4ccccc4O)nnc3N)CCC2C)n1]

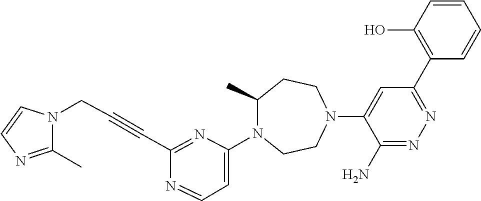 [SMILES: Cc1nccn1CC#Cc1nccc(N2CCN(c3cc(-c4ccccc4O)nnc3N)CC[C@@H]2C)n1]

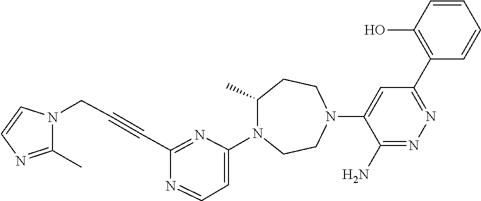 [SMILES: Cc1nccn1CC#Cc1nccc(N2CCN(c3cc(-c4ccccc4O)nnc3N)CC[C@H]2C)n1]

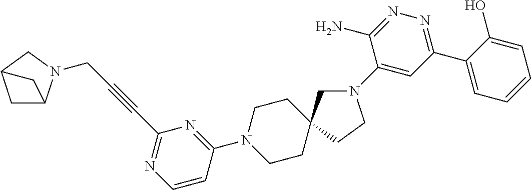

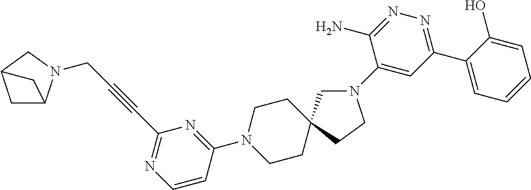

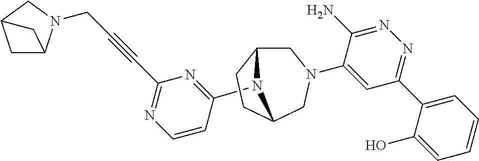 [SMILES: Nc1nnc(-c2ccccc2O)cc1N1C[C@H]2CC[C@@H](C1)N2c1ccnc(C#CCN2CC3CC2C3)n1]

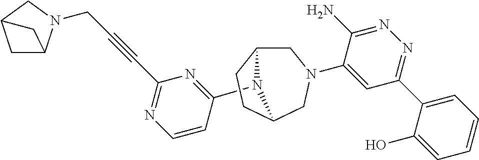 [SMILES: Nc1nnc(-c2ccccc2O)cc1N1C[C@H]2CC[C@@H](C1)N2c1ccnc(C#CCN2CC3CC2C3)n1]

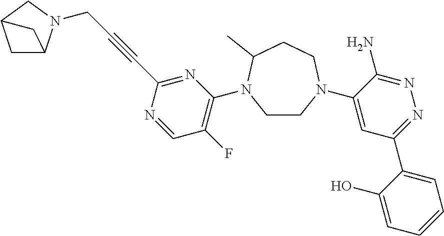 [SMILES: CC1CCN(c2cc(-c3ccccc3O)nnc2N)CCN1c1nc(C#CCN2CC3CC2C3)ncc1F]

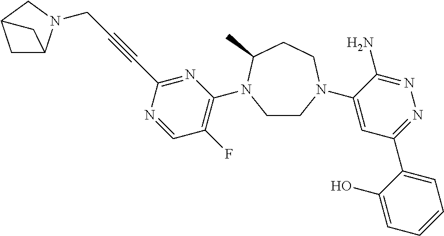 [SMILES: C[C@H]1CCN(c2cc(-c3ccccc3O)nnc2N)CCN1c1nc(C#CCN2CC3CC2C3)ncc1F]

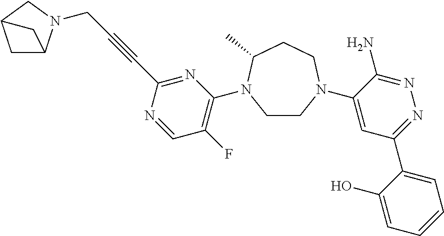 [SMILES: C[C@@H]1CCN(c2cc(-c3ccccc3O)nnc2N)CCN1c1nc(C#CCN2CC3CC2C3)ncc1F]

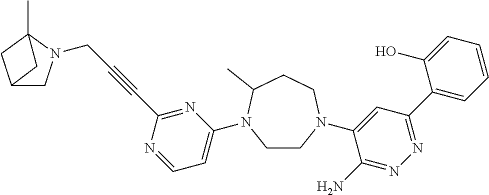 [SMILES: CC1CCN(c2cc(-c3ccccc3O)nnc2N)CCN1c1ccnc(C#CCN2CC3CC2(C)C3)n1]

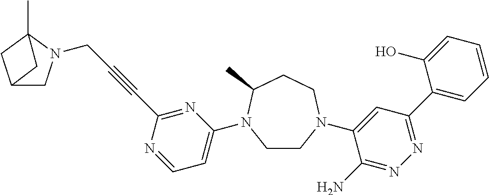 [SMILES: C[C@H]1CCN(c2cc(-c3ccccc3O)nnc2N)CCN1c1ccnc(C#CCN2CC3CC2(C)C3)n1]

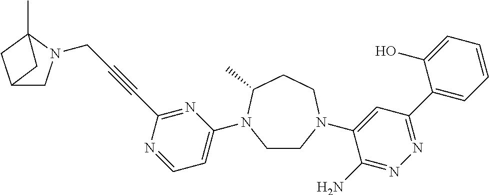 [SMILES: C[C@@H]1CCN(c2cc(-c3ccccc3O)nnc2N)CCN1c1ccnc(C#CCN2CC3CC2(C)C3)n1]

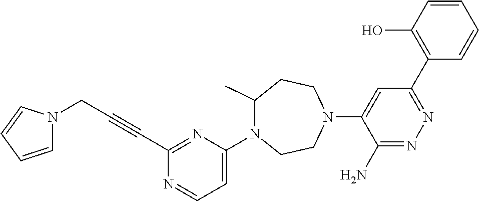 [SMILES: CC1CCN(c2cc(-c3ccccc3O)nnc2N)CCN1c1ccnc(C#CCn2cccc2)n1]

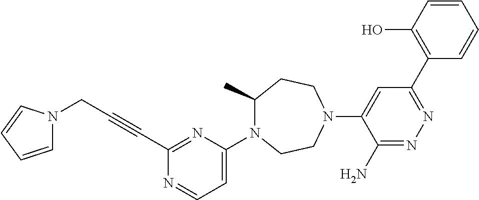 [SMILES: C[C@H]1CCN(c2cc(-c3ccccc3O)nnc2N)CCN1c1ccnc(C#CCn2cccc2)n1]

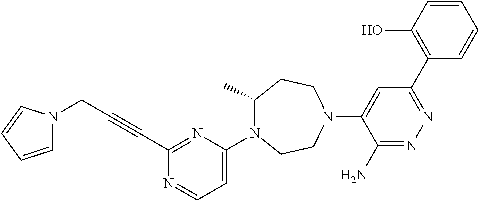 [SMILES: C[C@@H]1CCN(c2cc(-c3ccccc3O)nnc2N)CCN1c1ccnc(C#CCn2cccc2)n1]

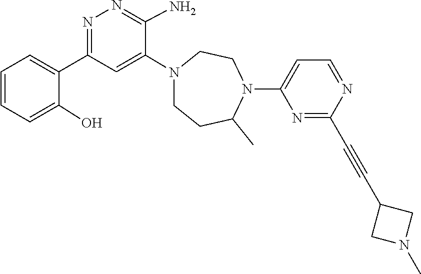 [SMILES: CC1CCN(c2cc(-c3ccccc3O)nnc2N)CCN1c1ccnc(C#CC2CN(C)C2)n1]

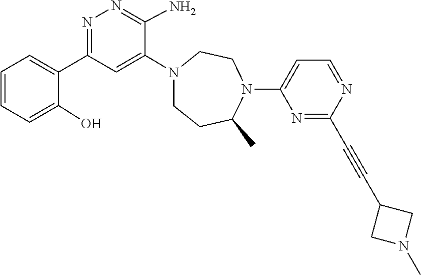 [SMILES: C[C@H]1CCN(c2cc(-c3ccccc3O)nnc2N)CCN1c1ccnc(C#CC2CN(C)C2)n1]

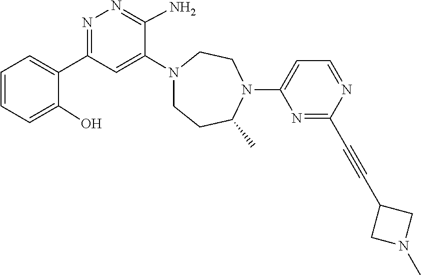 [SMILES: C[C@@H]1CCN(c2cc(-c3ccccc3O)nnc2N)CCN1c1ccnc(C#CC2CN(C)C2)n1]

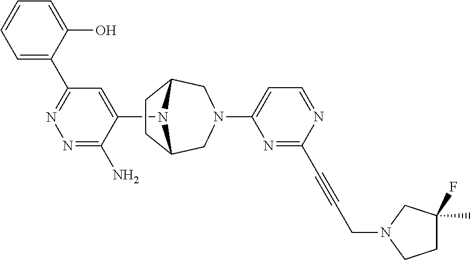 [SMILES: C[C@]1(F)CCN(CC#Cc2nccc(N3C[C@H]4CC[C@@H](C3)N4c3cc(-c4ccccc4O)nnc3N)n2)C1]

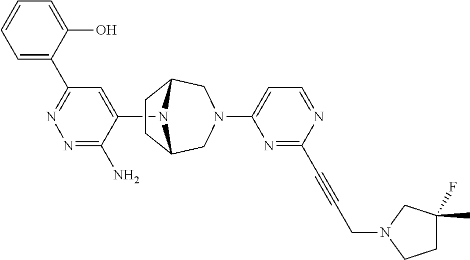 [SMILES: C[C@@]1(F)CCN(CC#Cc2nccc(N3C[C@H]4CC[C@@H](C3)N4c3cc(-c4ccccc4O)nnc3N)n2)C1]

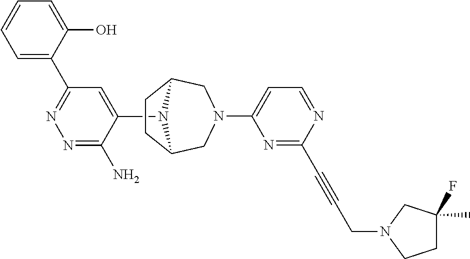 [SMILES: C[C@]1(F)CCN(CC#Cc2nccc(N3C[C@H]4CC[C@@H](C3)N4c3cc(-c4ccccc4O)nnc3N)n2)C1]

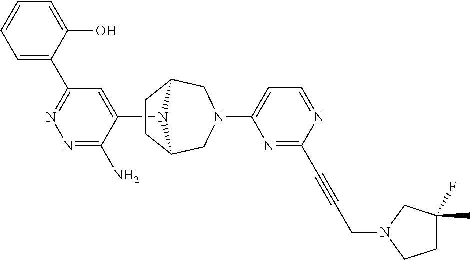 [SMILES: C[C@@]1(F)CCN(CC#Cc2nccc(N3C[C@H]4CC[C@@H](C3)N4c3cc(-c4ccccc4O)nnc3N)n2)C1]

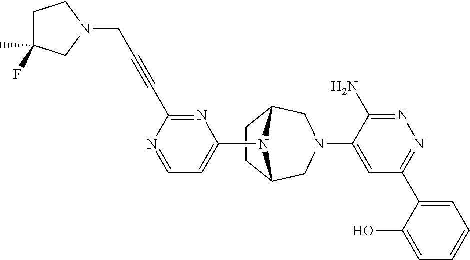 [SMILES: C[C@]1(F)CCN(CC#Cc2nccc(N3[C@@H]4CC[C@H]3CN(c3cc(-c5ccccc5O)nnc3N)C4)n2)C1]

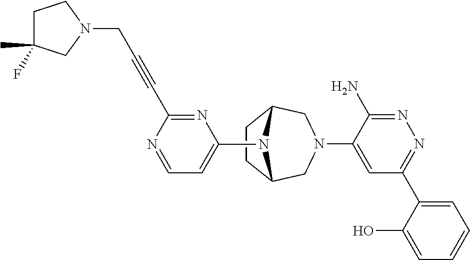 [SMILES: C[C@@]1(F)CCN(CC#Cc2nccc(N3[C@@H]4CC[C@H]3CN(c3cc(-c5ccccc5O)nnc3N)C4)n2)C1]

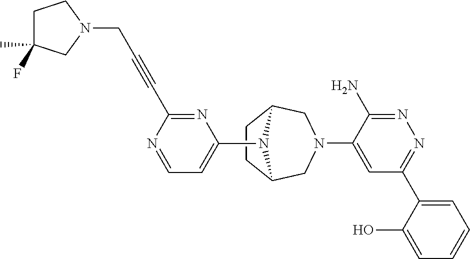 [SMILES: C[C@]1(F)CCN(CC#Cc2nccc(N3[C@@H]4CC[C@H]3CN(c3cc(-c5ccccc5O)nnc3N)C4)n2)C1]

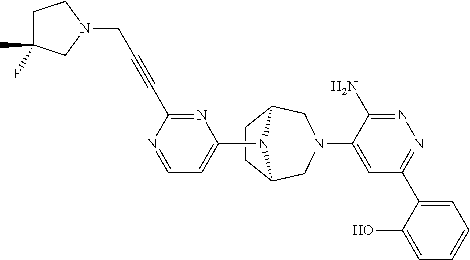 [SMILES: C[C@@]1(F)CCN(CC#Cc2nccc(N3[C@@H]4CC[C@H]3CN(c3cc(-c5ccccc5O)nnc3N)C4)n2)C1]

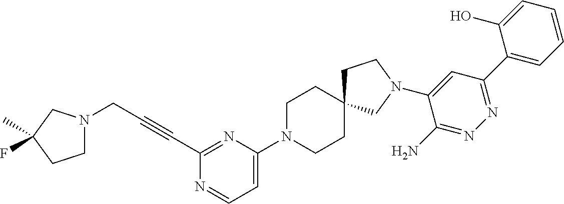

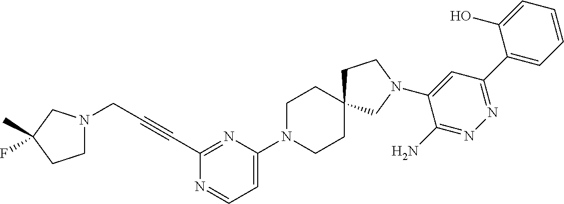

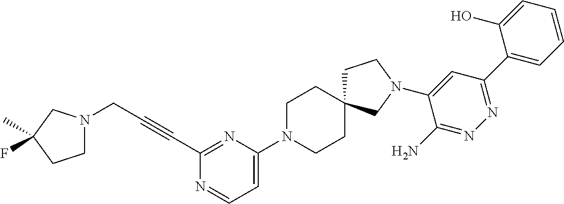

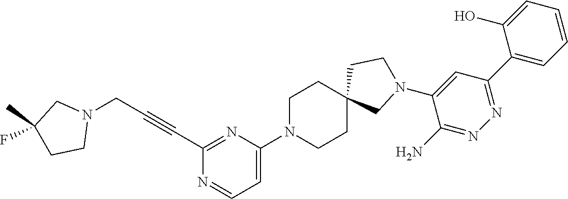

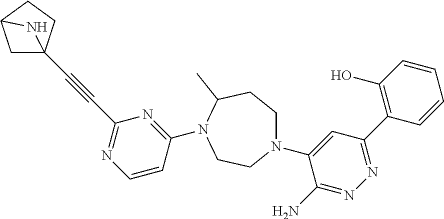 [SMILES: CC1CCN(c2cc(-c3ccccc3O)nnc2N)CCN1c1ccnc(C#CC23CCC(C2)N3)n1]

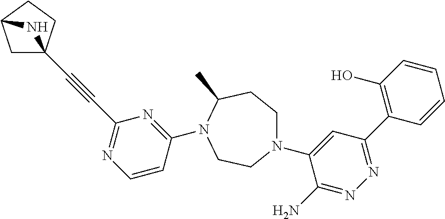 [SMILES: C[C@H]1CCN(c2cc(-c3ccccc3O)nnc2N)CCN1c1ccnc(C#C[C@@]23CC[C@@H](C2)N3)n1]

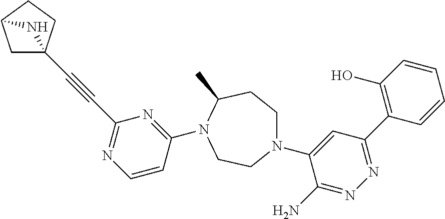 [SMILES: C[C@H]1CCN(c2cc(-c3ccccc3O)nnc2N)CCN1c1ccnc(C#C[C@]23CC[C@H](C2)N3)n1]

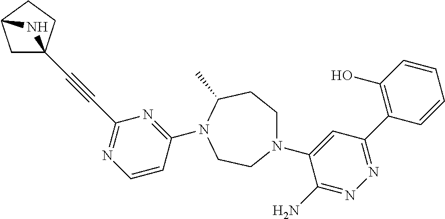 [SMILES: C[C@@H]1CCN(c2cc(-c3ccccc3O)nnc2N)CCN1c1ccnc(C#C[C@@]23CC[C@@H](C2)N3)n1]

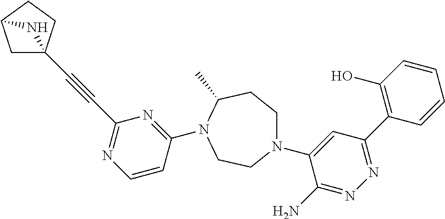 [SMILES: C[C@@H]1CCN(c2cc(-c3ccccc3O)nnc2N)CCN1c1ccnc(C#C[C@]23CC[C@H](C2)N3)n1]

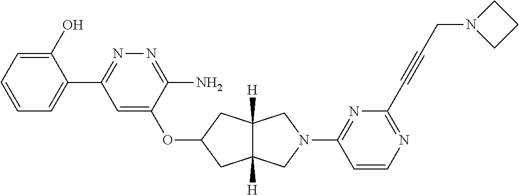 [SMILES: Nc1nnc(-c2ccccc2O)cc1OC1C[C@@H]2CN(c3ccnc(C#CCN4CCC4)n3)C[C@@H]2C1]

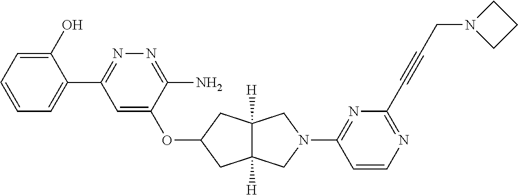 [SMILES: Nc1nnc(-c2ccccc2O)cc1OC1C[C@@H]2CN(c3ccnc(C#CCN4CCC4)n3)C[C@@H]2C1]

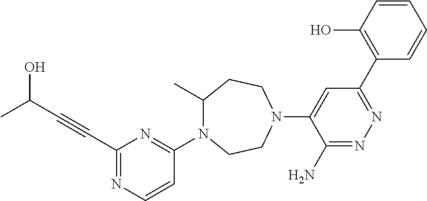 [SMILES: CC(O)C#Cc1nccc(N2CCN(c3cc(-c4ccccc4O)nnc3N)CCC2C)n1]

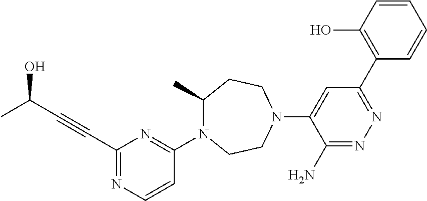 [SMILES: C[C@@H](O)C#Cc1nccc(N2CCN(c3cc(-c4ccccc4O)nnc3N)CC[C@@H]2C)n1]

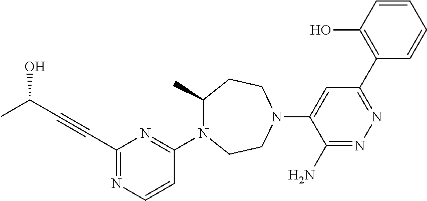 [SMILES: C[C@H](O)C#Cc1nccc(N2CCN(c3cc(-c4ccccc4O)nnc3N)CC[C@@H]2C)n1]

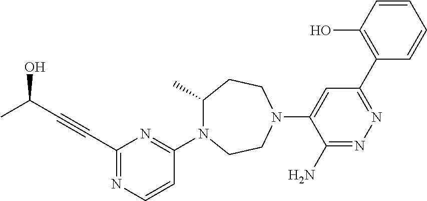 [SMILES: C[C@@H]1CCN(c2cc(-c3ccccc3O)nnc2N)CCN1c1ccnc(C#C[C@@H](C)O)n1]

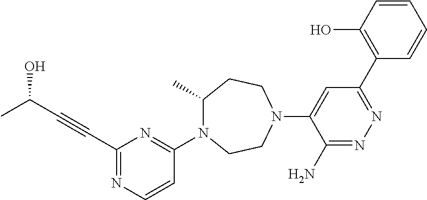 [SMILES: C[C@H](O)C#Cc1nccc(N2CCN(c3cc(-c4ccccc4O)nnc3N)CC[C@H]2C)n1]

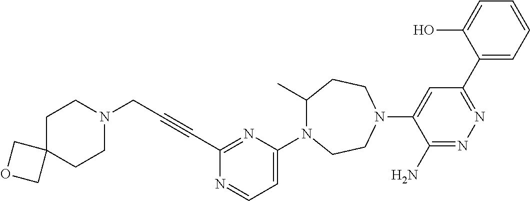 [SMILES: CC1CCN(c2cc(-c3ccccc3O)nnc2N)CCN1c1ccnc(C#CCN2CCC3(CC2)COC3)n1]

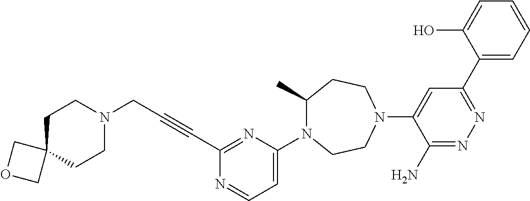

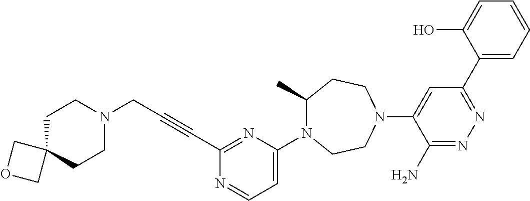

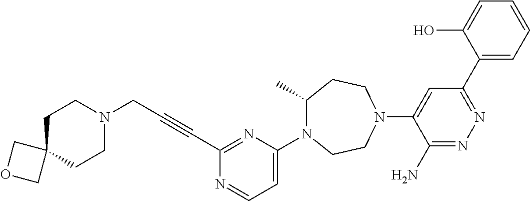

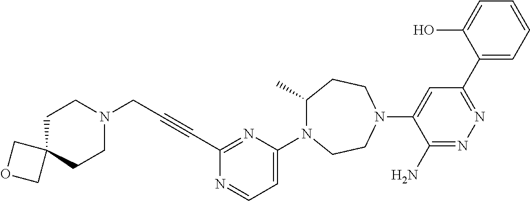

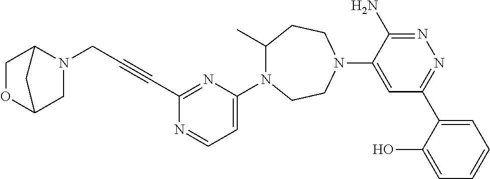 [SMILES: CC1CCN(c2cc(-c3ccccc3O)nnc2N)CCN1c1ccnc(C#CCN2CC3CC2CO3)n1]

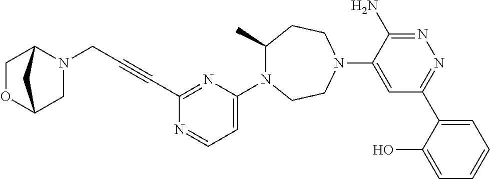 [SMILES: C[C@H]1CCN(c2cc(-c3ccccc3O)nnc2N)CCN1c1ccnc(C#CCN2C[C@H]3C[C@@H]2CO3)n1]

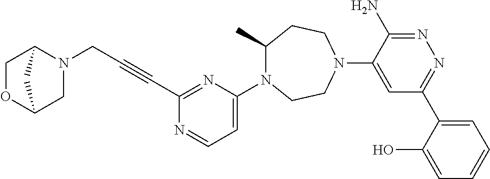 [SMILES: C[C@H]1CCN(c2cc(-c3ccccc3O)nnc2N)CCN1c1ccnc(C#CCN2C[C@@H]3C[C@H]2CO3)n1]

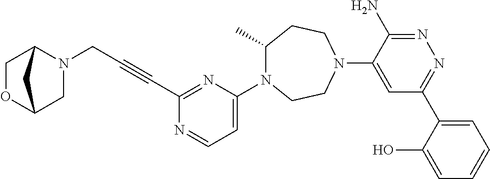 [SMILES: C[C@@H]1CCN(c2cc(-c3ccccc3O)nnc2N)CCN1c1ccnc(C#CCN2C[C@H]3C[C@@H]2CO3)n1]

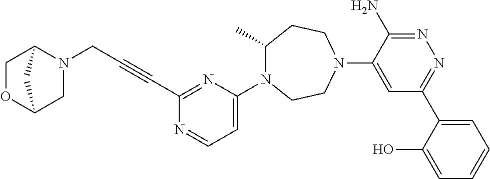 [SMILES: C[C@@H]1CCN(c2cc(-c3ccccc3O)nnc2N)CCN1c1ccnc(C#CCN2C[C@@H]3C[C@H]2CO3)n1]

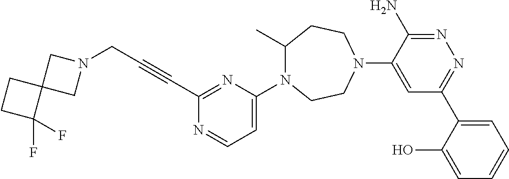 [SMILES: CC1CCN(c2cc(-c3ccccc3O)nnc2N)CCN1c1ccnc(C#CCN2CC3(CCC3(F)F)C2)n1]

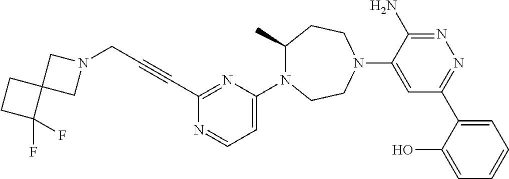 [SMILES: C[C@H]1CCN(c2cc(-c3ccccc3O)nnc2N)CCN1c1ccnc(C#CCN2CC3(CCC3(F)F)C2)n1]

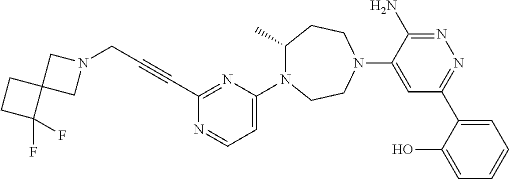 [SMILES: C[C@@H]1CCN(c2cc(-c3ccccc3O)nnc2N)CCN1c1ccnc(C#CCN2CC3(CCC3(F)F)C2)n1]

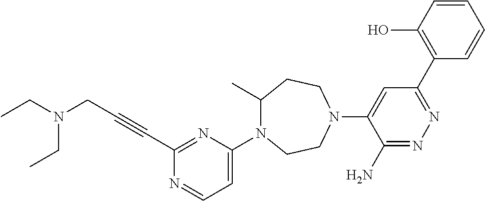 [SMILES: CCN(CC)CC#Cc1nccc(N2CCN(c3cc(-c4ccccc4O)nnc3N)CCC2C)n1]

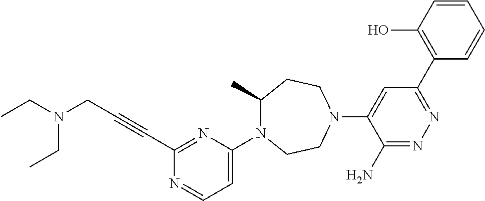 [SMILES: CCN(CC)CC#Cc1nccc(N2CCN(c3cc(-c4ccccc4O)nnc3N)CC[C@@H]2C)n1]

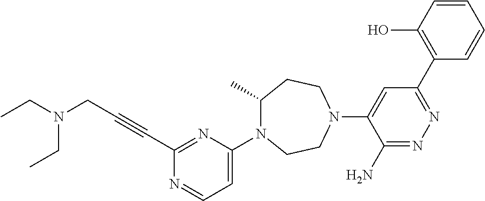 [SMILES: CCN(CC)CC#Cc1nccc(N2CCN(c3cc(-c4ccccc4O)nnc3N)CC[C@H]2C)n1]

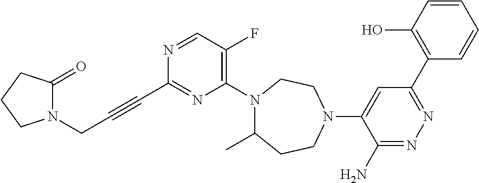 [SMILES: CC1CCN(c2cc(-c3ccccc3O)nnc2N)CCN1c1nc(C#CCN2CCCC2=O)ncc1F]

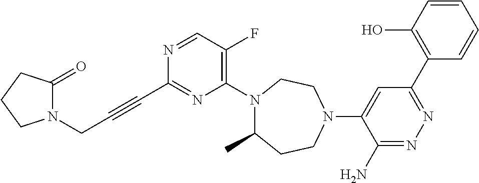 [SMILES: C[C@@H]1CCN(c2cc(-c3ccccc3O)nnc2N)CCN1c1nc(C#CCN2CCCC2=O)ncc1F]

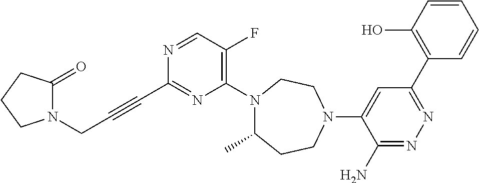 [SMILES: C[C@H]1CCN(c2cc(-c3ccccc3O)nnc2N)CCN1c1nc(C#CCN2CCCC2=O)ncc1F]

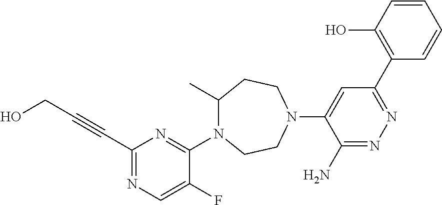 [SMILES: CC1CCN(c2cc(-c3ccccc3O)nnc2N)CCN1c1nc(C#CCO)ncc1F]

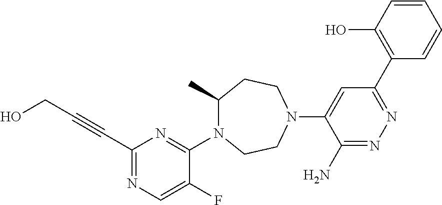 [SMILES: C[C@H]1CCN(c2cc(-c3ccccc3O)nnc2N)CCN1c1nc(C#CCO)ncc1F]

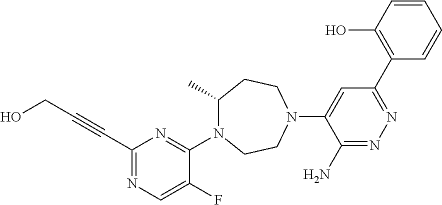 [SMILES: C[C@@H]1CCN(c2cc(-c3ccccc3O)nnc2N)CCN1c1nc(C#CCO)ncc1F]

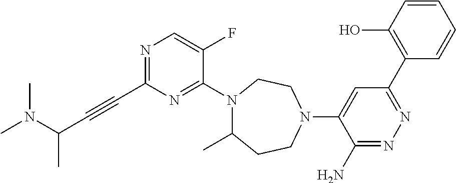 [SMILES: CC(C#Cc1ncc(F)c(N2CCN(c3cc(-c4ccccc4O)nnc3N)CCC2C)n1)N(C)C]

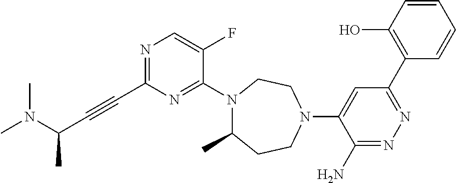 [SMILES: C[C@H](C#Cc1ncc(F)c(N2CCN(c3cc(-c4ccccc4O)nnc3N)CC[C@H]2C)n1)N(C)C]

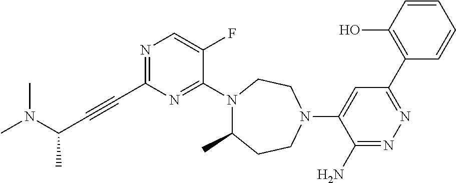 [SMILES: C[C@@H]1CCN(c2cc(-c3ccccc3O)nnc2N)CCN1c1nc(C#C[C@H](C)N(C)C)ncc1F]

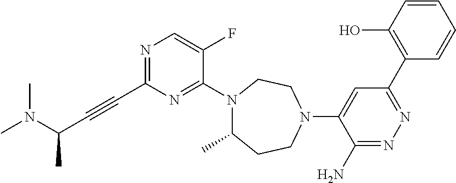 [SMILES: C[C@H](C#Cc1ncc(F)c(N2CCN(c3cc(-c4ccccc4O)nnc3N)CC[C@@H]2C)n1)N(C)C]

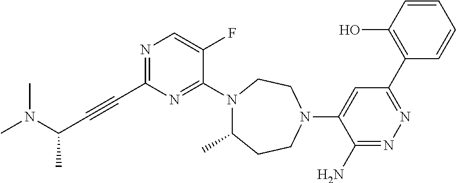 [SMILES: C[C@@H](C#Cc1ncc(F)c(N2CCN(c3cc(-c4ccccc4O)nnc3N)CC[C@@H]2C)n1)N(C)C]

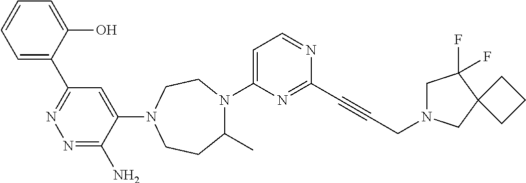 [SMILES: CC1CCN(c2cc(-c3ccccc3O)nnc2N)CCN1c1ccnc(C#CCN2CC(F)(F)C3(CCC3)C2)n1]

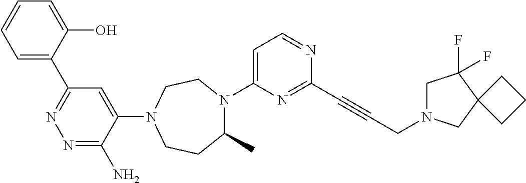 [SMILES: C[C@H]1CCN(c2cc(-c3ccccc3O)nnc2N)CCN1c1ccnc(C#CCN2CC(F)(F)C3(CCC3)C2)n1]

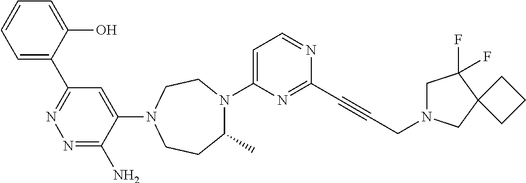 [SMILES: C[C@@H]1CCN(c2cc(-c3ccccc3O)nnc2N)CCN1c1ccnc(C#CCN2CC(F)(F)C3(CCC3)C2)n1]

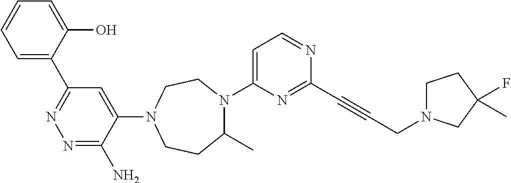 [SMILES: CC1CCN(c2cc(-c3ccccc3O)nnc2N)CCN1c1ccnc(C#CCN2CCC(C)(F)C2)n1]

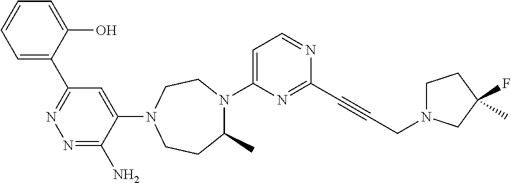 [SMILES: C[C@H]1CCN(c2cc(-c3ccccc3O)nnc2N)CCN1c1ccnc(C#CCN2CC[C@@](C)(F)C2)n1]

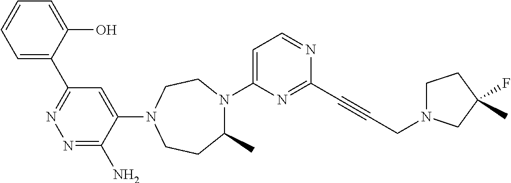 [SMILES: C[C@H]1CCN(c2cc(-c3ccccc3O)nnc2N)CCN1c1ccnc(C#CCN2CC[C@](C)(F)C2)n1]

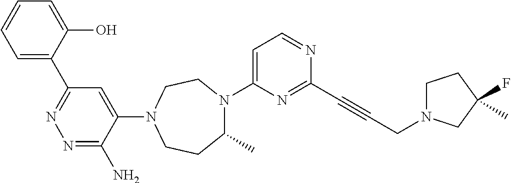 [SMILES: C[C@@H]1CCN(c2cc(-c3ccccc3O)nnc2N)CCN1c1ccnc(C#CCN2CC[C@@](C)(F)C2)n1]

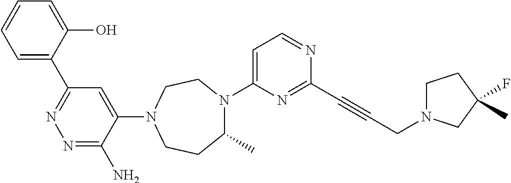 [SMILES: C[C@@H]1CCN(c2cc(-c3ccccc3O)nnc2N)CCN1c1ccnc(C#CCN2CC[C@](C)(F)C2)n1]

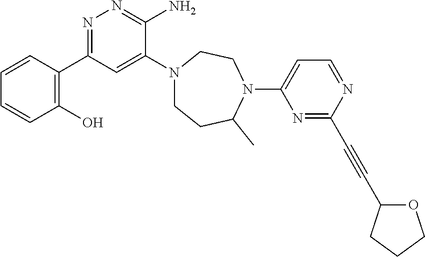 [SMILES: CC1CCN(c2cc(-c3ccccc3O)nnc2N)CCN1c1ccnc(C#CC2CCCO2)n1]

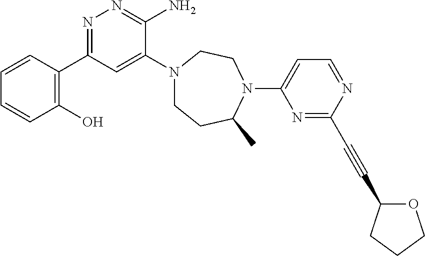 [SMILES: C[C@H]1CCN(c2cc(-c3ccccc3O)nnc2N)CCN1c1ccnc(C#C[C@@H]2CCCO2)n1]

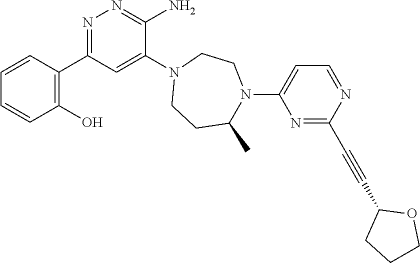 [SMILES: C[C@H]1CCN(c2cc(-c3ccccc3O)nnc2N)CCN1c1ccnc(C#C[C@H]2CCCO2)n1]

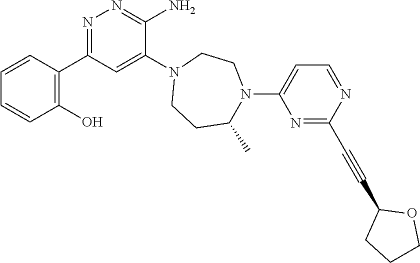 [SMILES: C[C@@H]1CCN(c2cc(-c3ccccc3O)nnc2N)CCN1c1ccnc(C#C[C@@H]2CCCO2)n1]

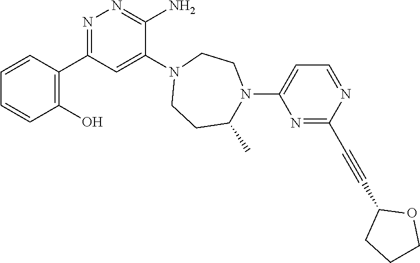 [SMILES: C[C@@H]1CCN(c2cc(-c3ccccc3O)nnc2N)CCN1c1ccnc(C#C[C@H]2CCCO2)n1]

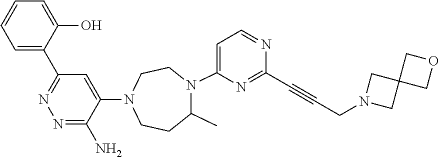 [SMILES: CC1CCN(c2cc(-c3ccccc3O)nnc2N)CCN1c1ccnc(C#CCN2CC3(COC3)C2)n1]

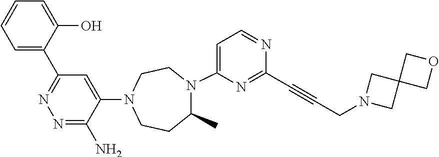 [SMILES: C[C@H]1CCN(c2cc(-c3ccccc3O)nnc2N)CCN1c1ccnc(C#CCN2CC3(COC3)C2)n1]

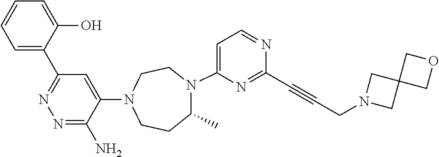 [SMILES: C[C@@H]1CCN(c2cc(-c3ccccc3O)nnc2N)CCN1c1ccnc(C#CCN2CC3(COC3)C2)n1]

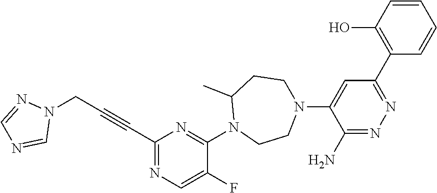 [SMILES: CC1CCN(c2cc(-c3ccccc3O)nnc2N)CCN1c1nc(C#CCn2cncn2)ncc1F]

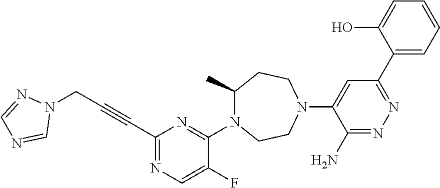 [SMILES: C[C@H]1CCN(c2cc(-c3ccccc3O)nnc2N)CCN1c1nc(C#CCn2cncn2)ncc1F]

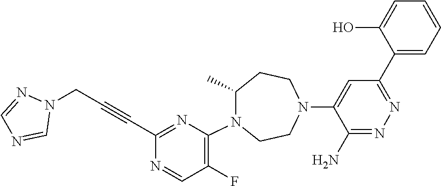 [SMILES: C[C@@H]1CCN(c2cc(-c3ccccc3O)nnc2N)CCN1c1nc(C#CCn2cncn2)ncc1F]

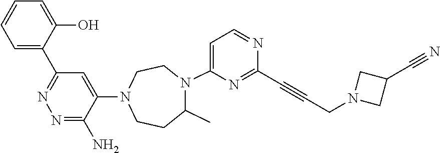 [SMILES: CC1CCN(c2cc(-c3ccccc3O)nnc2N)CCN1c1ccnc(C#CCN2CC(C#N)C2)n1]

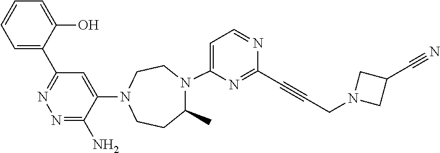 [SMILES: C[C@H]1CCN(c2cc(-c3ccccc3O)nnc2N)CCN1c1ccnc(C#CCN2CC(C#N)C2)n1]

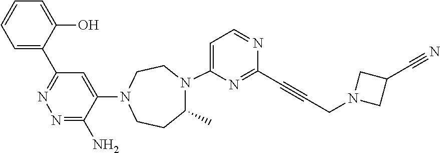 [SMILES: C[C@@H]1CCN(c2cc(-c3ccccc3O)nnc2N)CCN1c1ccnc(C#CCN2CC(C#N)C2)n1]

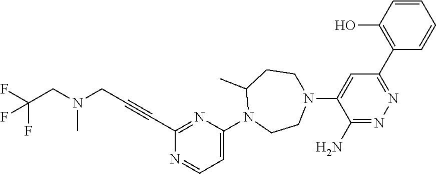 [SMILES: CC1CCN(c2cc(-c3ccccc3O)nnc2N)CCN1c1ccnc(C#CCN(C)CC(F)(F)F)n1]

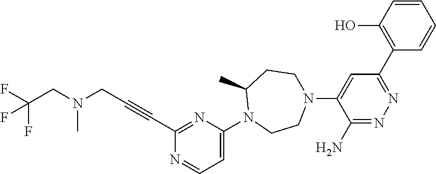 [SMILES: C[C@H]1CCN(c2cc(-c3ccccc3O)nnc2N)CCN1c1ccnc(C#CCN(C)CC(F)(F)F)n1]

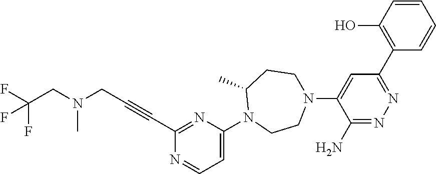 [SMILES: C[C@@H]1CCN(c2cc(-c3ccccc3O)nnc2N)CCN1c1ccnc(C#CCN(C)CC(F)(F)F)n1]

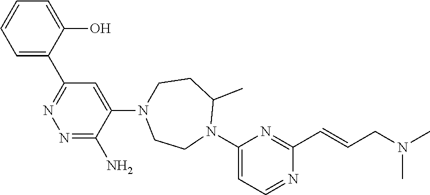 [SMILES: CC1CCN(c2cc(-c3ccccc3O)nnc2N)CCN1c1ccnc(/C=C/CN(C)C)n1]

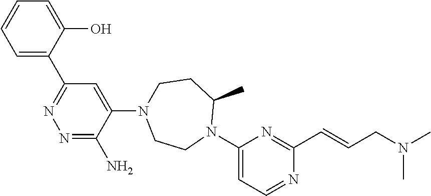 [SMILES: C[C@@H]1CCN(c2cc(-c3ccccc3O)nnc2N)CCN1c1ccnc(/C=C/CN(C)C)n1]

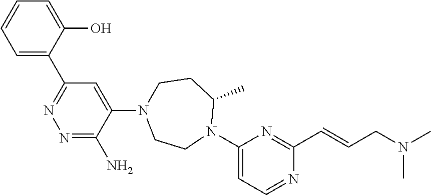 [SMILES: C[C@H]1CCN(c2cc(-c3ccccc3O)nnc2N)CCN1c1ccnc(/C=C/CN(C)C)n1]

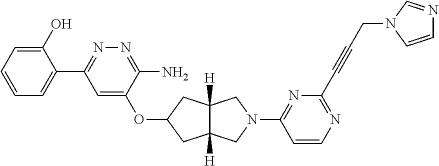 [SMILES: Nc1nnc(-c2ccccc2O)cc1OC1C[C@@H]2CN(c3ccnc(C#CCn4ccnc4)n3)C[C@@H]2C1]

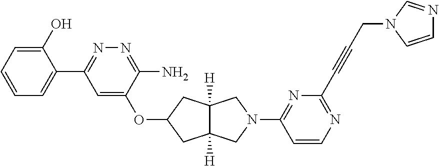 [SMILES: Nc1nnc(-c2ccccc2O)cc1OC1C[C@@H]2CN(c3ccnc(C#CCn4ccnc4)n3)C[C@@H]2C1]

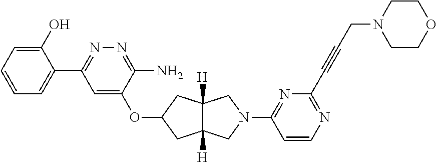 [SMILES: Nc1nnc(-c2ccccc2O)cc1OC1C[C@@H]2CN(c3ccnc(C#CCN4CCOCC4)n3)C[C@@H]2C1]

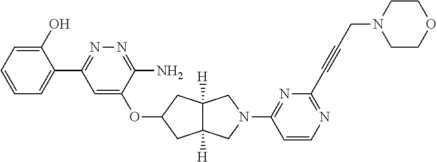 [SMILES: Nc1nnc(-c2ccccc2O)cc1OC1C[C@@H]2CN(c3ccnc(C#CCN4CCOCC4)n3)C[C@@H]2C1]

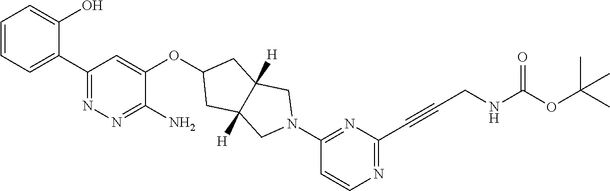 [SMILES: CC(C)(C)OC(=O)NCC#Cc1nccc(N2C[C@H]3CC(Oc4cc(-c5ccccc5O)nnc4N)C[C@H]3C2)n1]

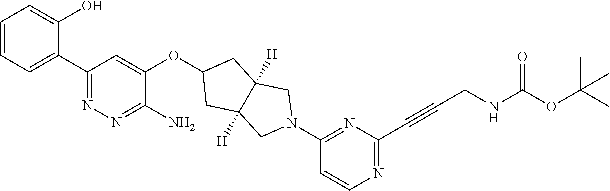 [SMILES: CC(C)(C)OC(=O)NCC#Cc1nccc(N2C[C@H]3CC(Oc4cc(-c5ccccc5O)nnc4N)C[C@H]3C2)n1]

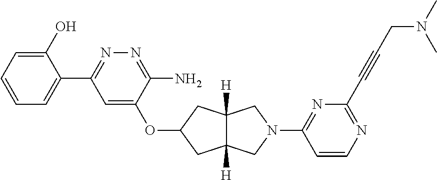 [SMILES: CN(C)CC#Cc1nccc(N2C[C@H]3CC(Oc4cc(-c5ccccc5O)nnc4N)C[C@H]3C2)n1]

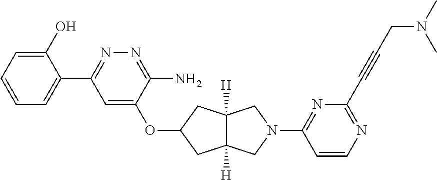 [SMILES: CN(C)CC#Cc1nccc(N2C[C@H]3CC(Oc4cc(-c5ccccc5O)nnc4N)C[C@H]3C2)n1]

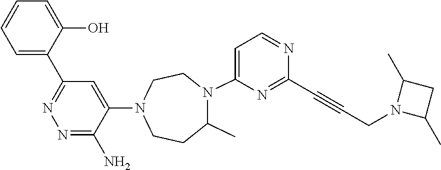 [SMILES: CC1CCN(c2cc(-c3ccccc3O)nnc2N)CCN1c1ccnc(C#CCN2C(C)CC2C)n1]

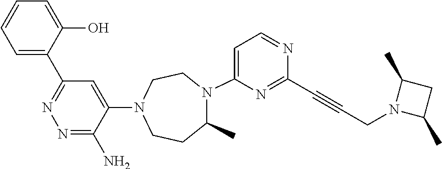 [SMILES: C[C@@H]1C[C@H](C)N1CC#Cc1nccc(N2CCN(c3cc(-c4ccccc4O)nnc3N)CC[C@@H]2C)n1]

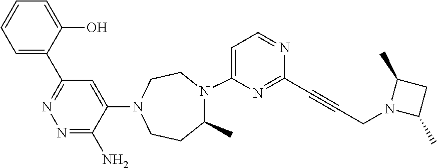 [SMILES: C[C@H]1CCN(c2cc(-c3ccccc3O)nnc2N)CCN1c1ccnc(C#CCN2[C@@H](C)C[C@@H]2C)n1]

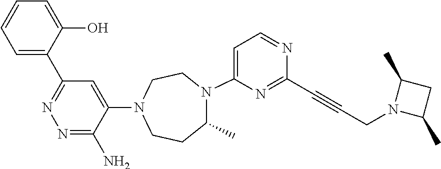 [SMILES: C[C@@H]1CCN(c2cc(-c3ccccc3O)nnc2N)CCN1c1ccnc(C#CCN2[C@H](C)C[C@@H]2C)n1]

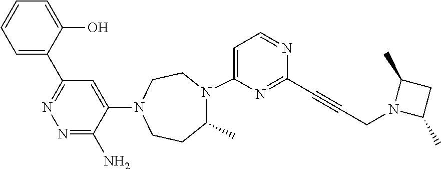 [SMILES: C[C@@H]1CCN(c2cc(-c3ccccc3O)nnc2N)CCN1c1ccnc(C#CCN2[C@@H](C)C[C@@H]2C)n1]

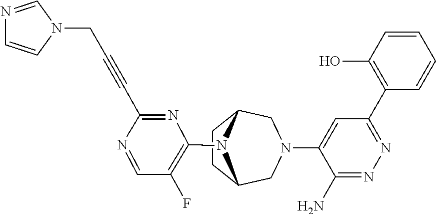 [SMILES: Nc1nnc(-c2ccccc2O)cc1N1C[C@H]2CC[C@@H](C1)N2c1nc(C#CCn2ccnc2)ncc1F]

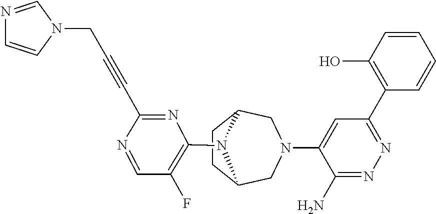

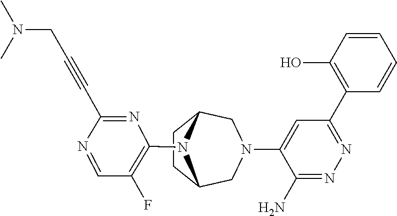 [SMILES: CN(C)CC#Cc1ncc(F)c(N2[C@@H]3CC[C@H]2CN(c2cc(-c4ccccc4O)nnc2N)C3)n1]

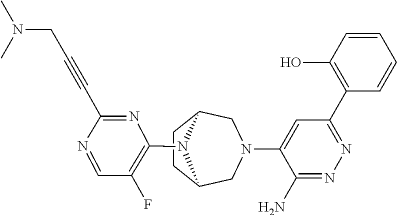 [SMILES: CN(C)CC#Cc1ncc(F)c(N2[C@@H]3CC[C@H]2CN(c2cc(-c4ccccc4O)nnc2N)C3)n1]

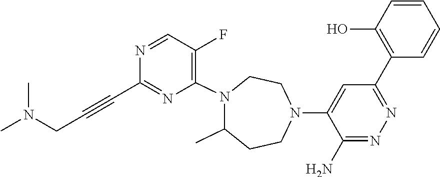 [SMILES: CC1CCN(c2cc(-c3ccccc3O)nnc2N)CCN1c1nc(C#CCN(C)C)ncc1F]

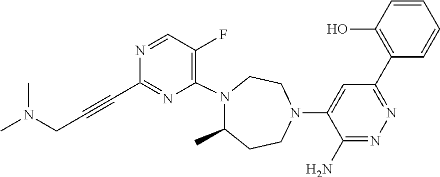 [SMILES: C[C@@H]1CCN(c2cc(-c3ccccc3O)nnc2N)CCN1c1nc(C#CCN(C)C)ncc1F]

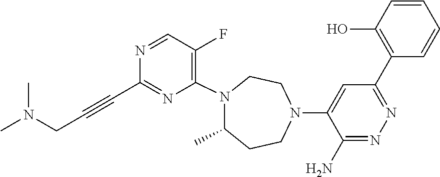 [SMILES: C[C@H]1CCN(c2cc(-c3ccccc3O)nnc2N)CCN1c1nc(C#CCN(C)C)ncc1F]

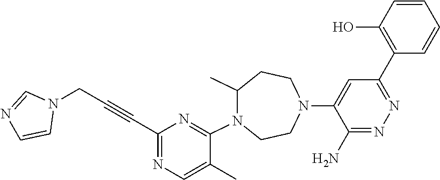 [SMILES: Cc1cnc(C#CCn2ccnc2)nc1N1CCN(c2cc(-c3ccccc3O)nnc2N)CCC1C]

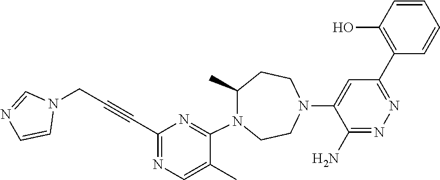 [SMILES: Cc1cnc(C#CCn2ccnc2)nc1N1CCN(c2cc(-c3ccccc3O)nnc2N)CC[C@@H]1C]

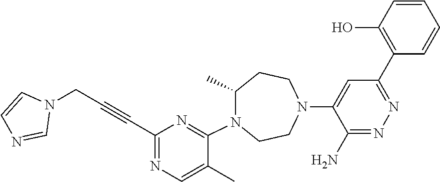 [SMILES: Cc1cnc(C#CCn2ccnc2)nc1N1CCN(c2cc(-c3ccccc3O)nnc2N)CC[C@H]1C]

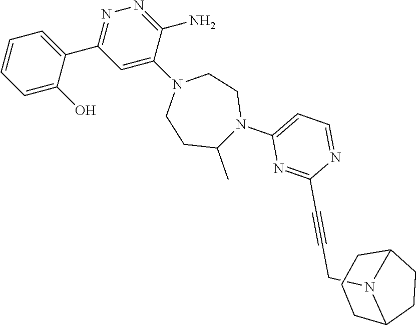 [SMILES: CC1CCN(c2cc(-c3ccccc3O)nnc2N)CCN1c1ccnc(C#CCN2C3CCCC2CC3)n1]

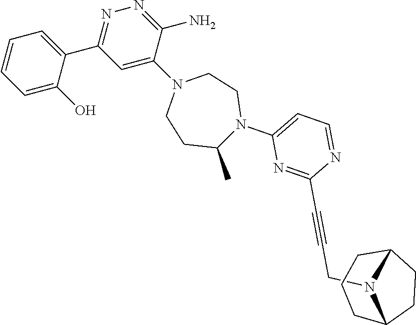 [SMILES: C[C@H]1CCN(c2cc(-c3ccccc3O)nnc2N)CCN1c1ccnc(C#CCN2[C@@H]3CCC[C@H]2CC3)n1]

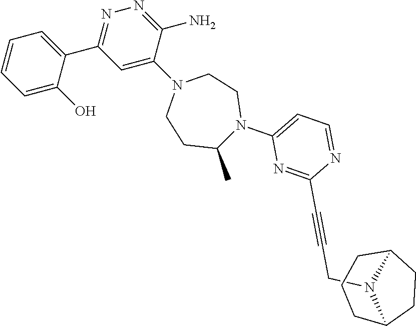 [SMILES: C[C@H]1CCN(c2cc(-c3ccccc3O)nnc2N)CCN1c1ccnc(C#CCN2[C@@H]3CCC[C@H]2CC3)n1]

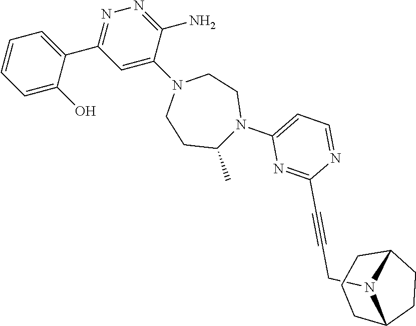 [SMILES: C[C@@H]1CCN(c2cc(-c3ccccc3O)nnc2N)CCN1c1ccnc(C#CCN2[C@@H]3CCC[C@H]2CC3)n1]

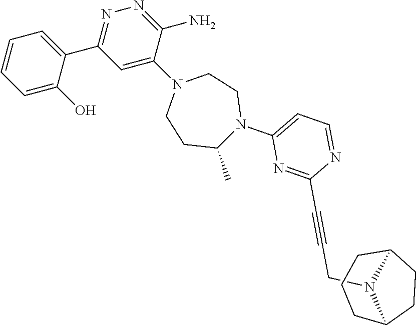 [SMILES: C[C@@H]1CCN(c2cc(-c3ccccc3O)nnc2N)CCN1c1ccnc(C#CCN2[C@@H]3CCC[C@H]2CC3)n1]

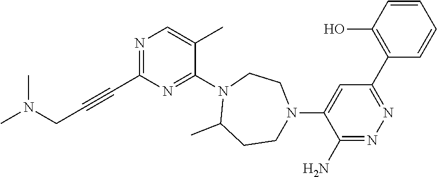 [SMILES: Cc1cnc(C#CCN(C)C)nc1N1CCN(c2cc(-c3ccccc3O)nnc2N)CCC1C]

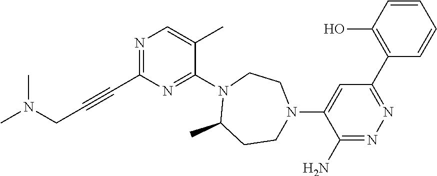 [SMILES: Cc1cnc(C#CCN(C)C)nc1N1CCN(c2cc(-c3ccccc3O)nnc2N)CC[C@H]1C]

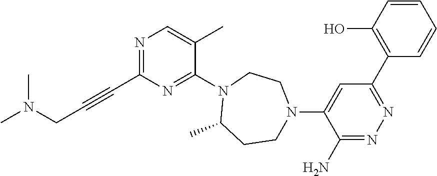 [SMILES: Cc1cnc(C#CCN(C)C)nc1N1CCN(c2cc(-c3ccccc3O)nnc2N)CC[C@@H]1C]

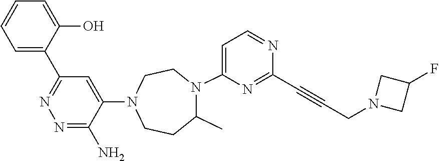 [SMILES: CC1CCN(c2cc(-c3ccccc3O)nnc2N)CCN1c1ccnc(C#CCN2CC(F)C2)n1]

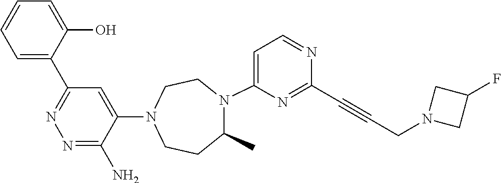 [SMILES: C[C@H]1CCN(c2cc(-c3ccccc3O)nnc2N)CCN1c1ccnc(C#CCN2CC(F)C2)n1]

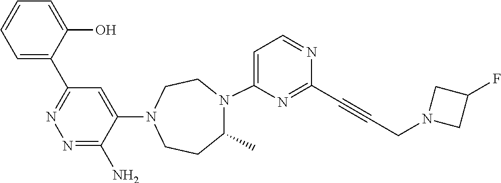 [SMILES: C[C@@H]1CCN(c2cc(-c3ccccc3O)nnc2N)CCN1c1ccnc(C#CCN2CC(F)C2)n1]

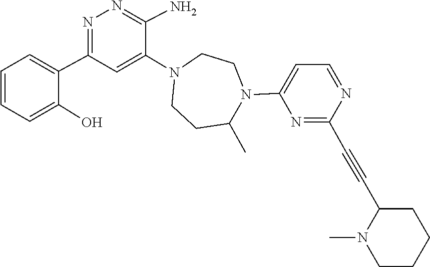 [SMILES: CC1CCN(c2cc(-c3ccccc3O)nnc2N)CCN1c1ccnc(C#CC2CCCCN2C)n1]

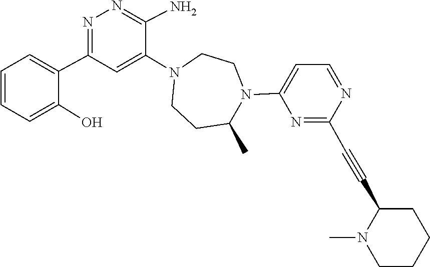 [SMILES: C[C@H]1CCN(c2cc(-c3ccccc3O)nnc2N)CCN1c1ccnc(C#C[C@H]2CCCCN2C)n1]

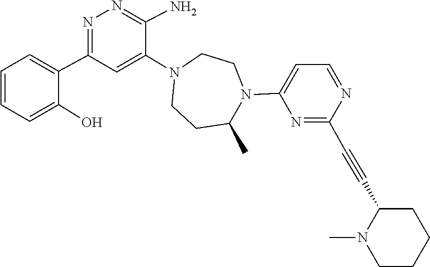 [SMILES: C[C@H]1CCN(c2cc(-c3ccccc3O)nnc2N)CCN1c1ccnc(C#C[C@@H]2CCCCN2C)n1]

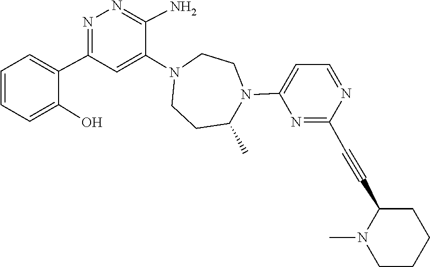 [SMILES: C[C@@H]1CCN(c2cc(-c3ccccc3O)nnc2N)CCN1c1ccnc(C#C[C@H]2CCCCN2C)n1]

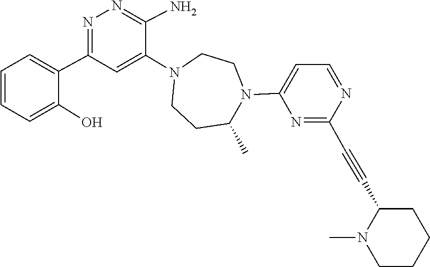 [SMILES: C[C@@H]1CCN(c2cc(-c3ccccc3O)nnc2N)CCN1c1ccnc(C#C[C@@H]2CCCCN2C)n1]

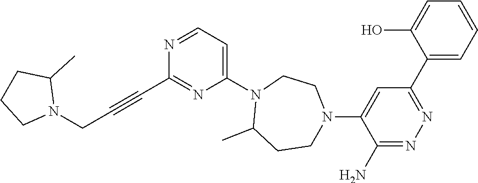 [SMILES: CC1CCCN1CC#Cc1nccc(N2CCN(c3cc(-c4ccccc4O)nnc3N)CCC2C)n1]

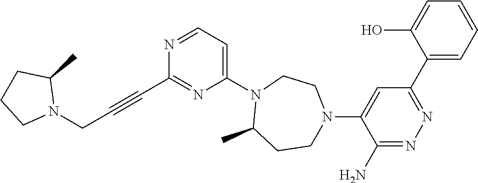 [SMILES: C[C@@H]1CCCN1CC#Cc1nccc(N2CCN(c3cc(-c4ccccc4O)nnc3N)CC[C@H]2C)n1]

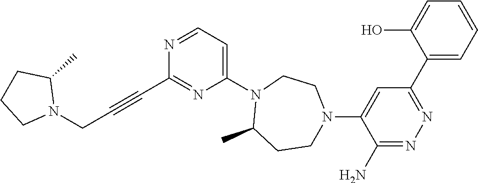 [SMILES: C[C@@H]1CCN(c2cc(-c3ccccc3O)nnc2N)CCN1c1ccnc(C#CCN2CCC[C@@H]2C)n1]

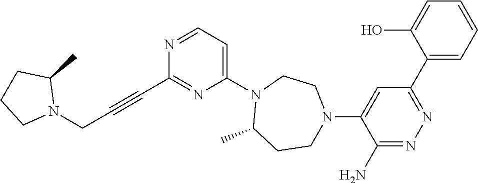 [SMILES: C[C@@H]1CCCN1CC#Cc1nccc(N2CCN(c3cc(-c4ccccc4O)nnc3N)CC[C@@H]2C)n1]

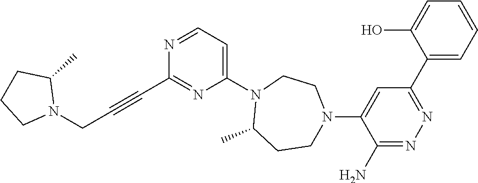 [SMILES: C[C@H]1CCCN1CC#Cc1nccc(N2CCN(c3cc(-c4ccccc4O)nnc3N)CC[C@@H]2C)n1]

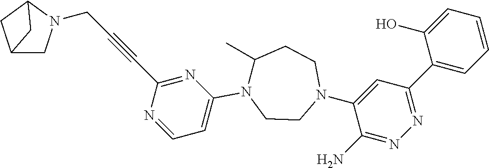 [SMILES: CC1CCN(c2cc(-c3ccccc3O)nnc2N)CCN1c1ccnc(C#CCN2CC3CC2C3)n1]

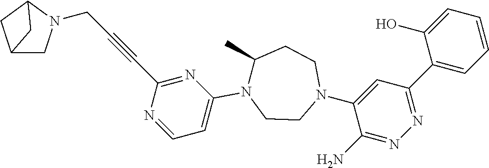 [SMILES: C[C@H]1CCN(c2cc(-c3ccccc3O)nnc2N)CCN1c1ccnc(C#CCN2CC3CC2C3)n1]

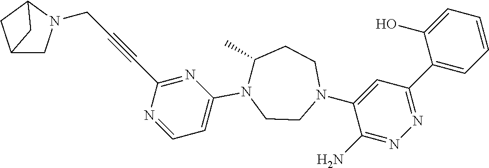 [SMILES: C[C@@H]1CCN(c2cc(-c3ccccc3O)nnc2N)CCN1c1ccnc(C#CCN2CC3CC2C3)n1]

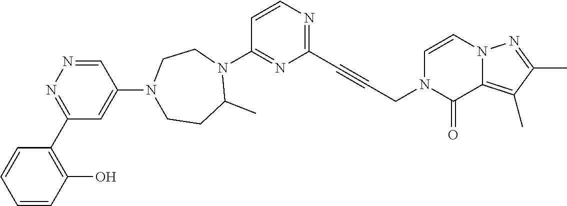 [SMILES: Cc1nn2ccn(CC#Cc3nccc(N4CCN(c5cnnc(-c6ccccc6O)c5)CCC4C)n3)c(=O)c2c1C]

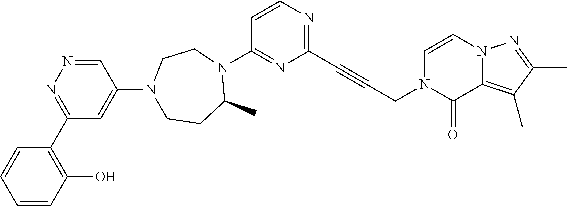 [SMILES: Cc1nn2ccn(CC#Cc3nccc(N4CCN(c5cnnc(-c6ccccc6O)c5)CC[C@@H]4C)n3)c(=O)c2c1C]

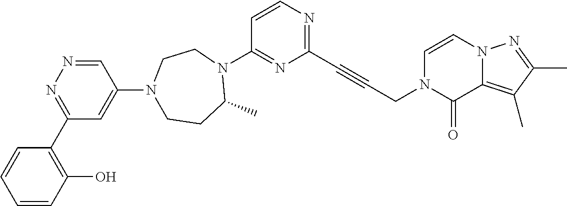 [SMILES: Cc1nn2ccn(CC#Cc3nccc(N4CCN(c5cnnc(-c6ccccc6O)c5)CC[C@H]4C)n3)c(=O)c2c1C]

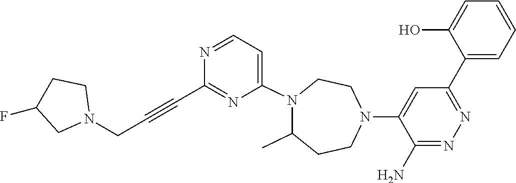 [SMILES: CC1CCN(c2cc(-c3ccccc3O)nnc2N)CCN1c1ccnc(C#CCN2CCC(F)C2)n1]

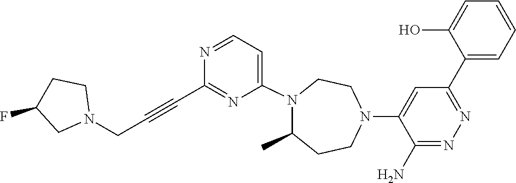 [SMILES: C[C@@H]1CCN(c2cc(-c3ccccc3O)nnc2N)CCN1c1ccnc(C#CCN2CC[C@H](F)C2)n1]

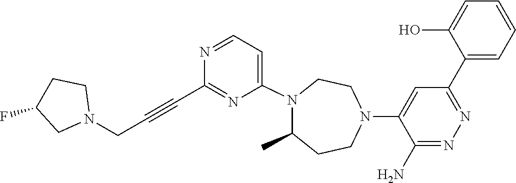 [SMILES: C[C@@H]1CCN(c2cc(-c3ccccc3O)nnc2N)CCN1c1ccnc(C#CCN2CC[C@@H](F)C2)n1]

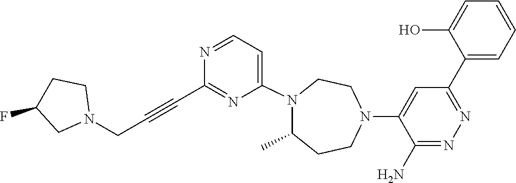 [SMILES: C[C@H]1CCN(c2cc(-c3ccccc3O)nnc2N)CCN1c1ccnc(C#CCN2CC[C@H](F)C2)n1]

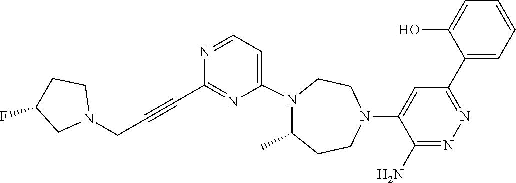 [SMILES: C[C@H]1CCN(c2cc(-c3ccccc3O)nnc2N)CCN1c1ccnc(C#CCN2CC[C@@H](F)C2)n1]

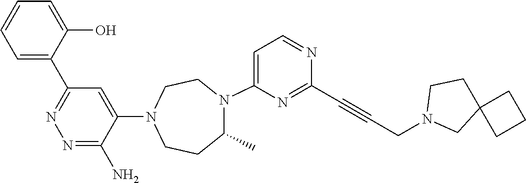 [SMILES: C[C@@H]1CCN(c2cc(-c3ccccc3O)nnc2N)CCN1c1ccnc(C#CCN2CCC3(CCC3)C2)n1]

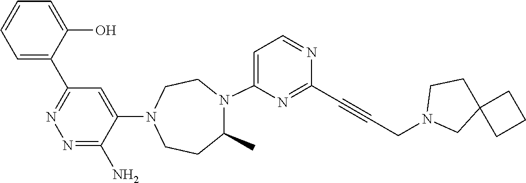 [SMILES: C[C@H]1CCN(c2cc(-c3ccccc3O)nnc2N)CCN1c1ccnc(C#CCN2CCC3(CCC3)C2)n1]

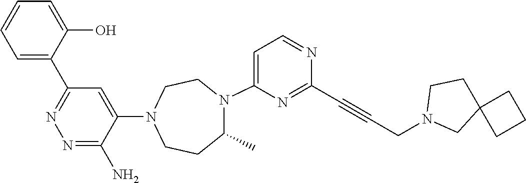 [SMILES: C[C@@H]1CCN(c2cc(-c3ccccc3O)nnc2N)CCN1c1ccnc(C#CCN2CCC3(CCC3)C2)n1]

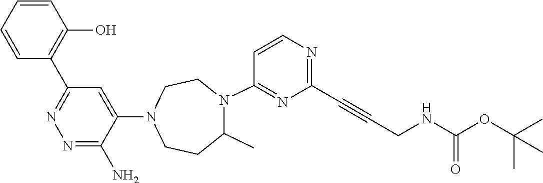 [SMILES: CC1CCN(c2cc(-c3ccccc3O)nnc2N)CCN1c1ccnc(C#CCNC(=O)OC(C)(C)C)n1]

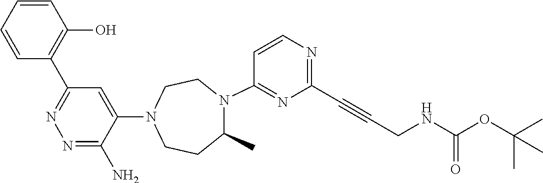 [SMILES: C[C@H]1CCN(c2cc(-c3ccccc3O)nnc2N)CCN1c1ccnc(C#CCNC(=O)OC(C)(C)C)n1]

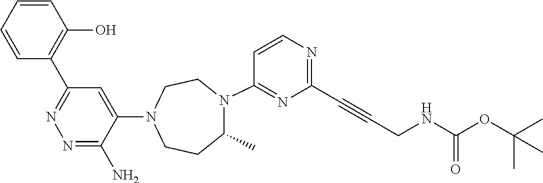 [SMILES: C[C@@H]1CCN(c2cc(-c3ccccc3O)nnc2N)CCN1c1ccnc(C#CCNC(=O)OC(C)(C)C)n1]

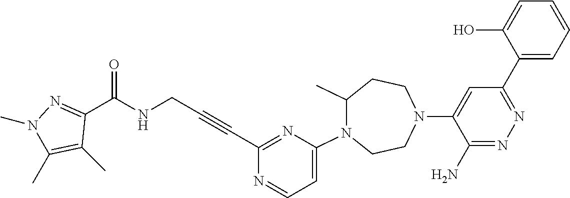 [SMILES: Cc1c(C(=O)NCC#Cc2nccc(N3CCN(c4cc(-c5ccccc5O)nnc4N)CCC3C)n2)nn(C)c1C]

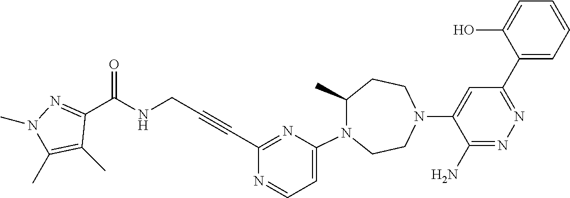 [SMILES: Cc1c(C(=O)NCC#Cc2nccc(N3CCN(c4cc(-c5ccccc5O)nnc4N)CC[C@@H]3C)n2)nn(C)c1C]

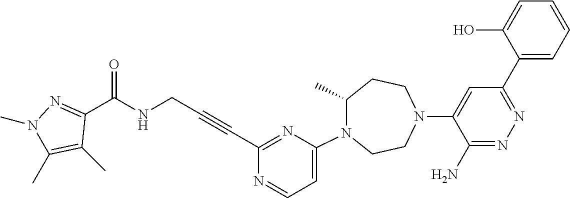 [SMILES: Cc1c(C(=O)NCC#Cc2nccc(N3CCN(c4cc(-c5ccccc5O)nnc4N)CC[C@H]3C)n2)nn(C)c1C]

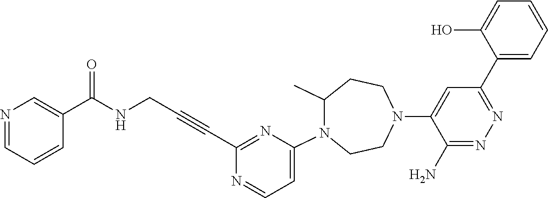 [SMILES: CC1CCN(c2cc(-c3ccccc3O)nnc2N)CCN1c1ccnc(C#CCNC(=O)c2cccnc2)n1]

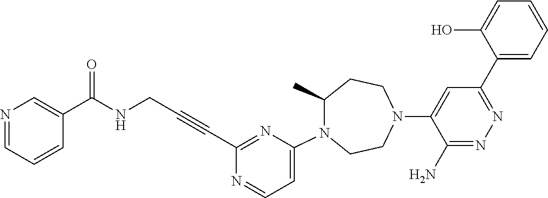 [SMILES: C[C@H]1CCN(c2cc(-c3ccccc3O)nnc2N)CCN1c1ccnc(C#CCNC(=O)c2cccnc2)n1]

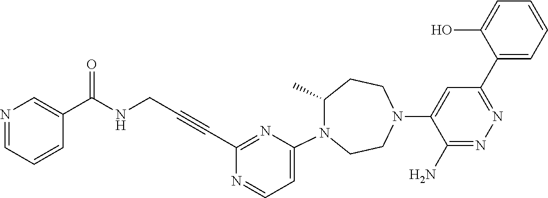 [SMILES: C[C@@H]1CCN(c2cc(-c3ccccc3O)nnc2N)CCN1c1ccnc(C#CCNC(=O)c2cccnc2)n1]

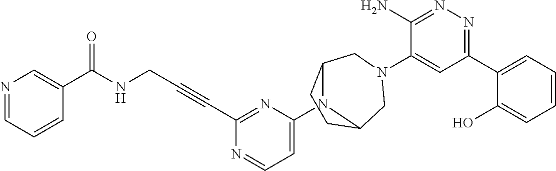 [SMILES: Nc1nnc(-c2ccccc2O)cc1N1CC2CCC(C1)N2c1ccnc(C#CCNC(=O)c2cccnc2)n1]

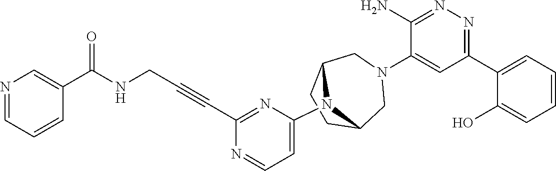 [SMILES: Nc1nnc(-c2ccccc2O)cc1N1C[C@H]2CC[C@@H](C1)N2c1ccnc(C#CCNC(=O)c2cccnc2)n1]

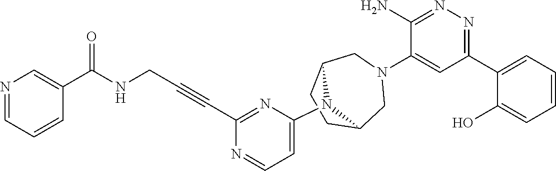 [SMILES: Nc1nnc(-c2ccccc2O)cc1N1C[C@H]2CC[C@@H](C1)N2c1ccnc(C#CCNC(=O)c2cccnc2)n1]

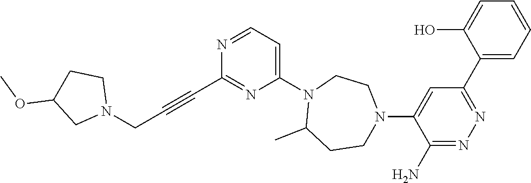 [SMILES: COC1CCN(CC#Cc2nccc(N3CCN(c4cc(-c5ccccc5O)nnc4N)CCC3C)n2)C1]

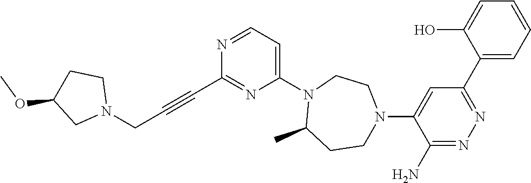 [SMILES: CO[C@H]1CCN(CC#Cc2nccc(N3CCN(c4cc(-c5ccccc5O)nnc4N)CC[C@H]3C)n2)C1]

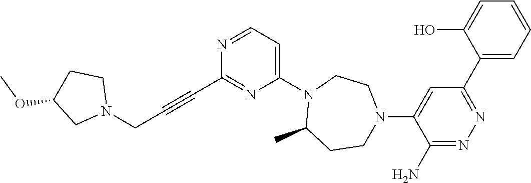 [SMILES: CO[C@@H]1CCN(CC#Cc2nccc(N3CCN(c4cc(-c5ccccc5O)nnc4N)CC[C@H]3C)n2)C1]

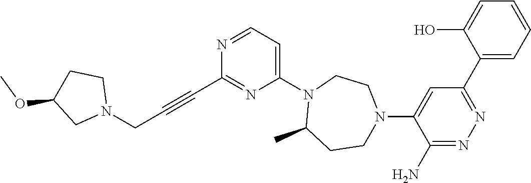 [SMILES: CO[C@H]1CCN(CC#Cc2nccc(N3CCN(c4cc(-c5ccccc5O)nnc4N)CC[C@H]3C)n2)C1]

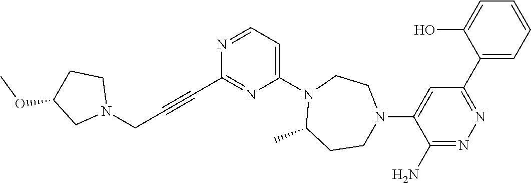 [SMILES: CO[C@@H]1CCN(CC#Cc2nccc(N3CCN(c4cc(-c5ccccc5O)nnc4N)CC[C@@H]3C)n2)C1]

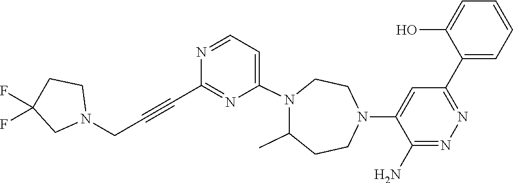 [SMILES: CC1CCN(c2cc(-c3ccccc3O)nnc2N)CCN1c1ccnc(C#CCN2CCC(F)(F)C2)n1]

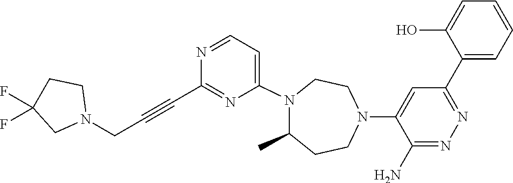 [SMILES: C[C@@H]1CCN(c2cc(-c3ccccc3O)nnc2N)CCN1c1ccnc(C#CCN2CCC(F)(F)C2)n1]

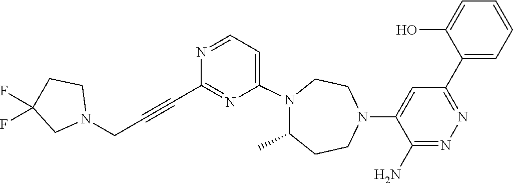 [SMILES: C[C@H]1CCN(c2cc(-c3ccccc3O)nnc2N)CCN1c1ccnc(C#CCN2CCC(F)(F)C2)n1]

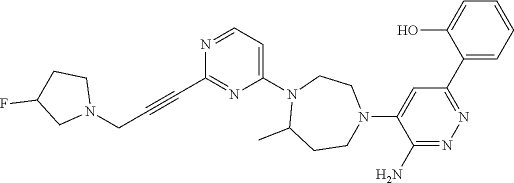 [SMILES: CC1CCN(c2cc(-c3ccccc3O)nnc2N)CCN1c1ccnc(C#CCN2CCC(F)C2)n1]

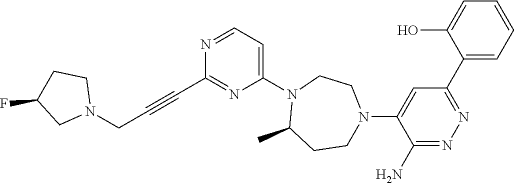 [SMILES: C[C@@H]1CCN(c2cc(-c3ccccc3O)nnc2N)CCN1c1ccnc(C#CCN2CC[C@H](F)C2)n1]

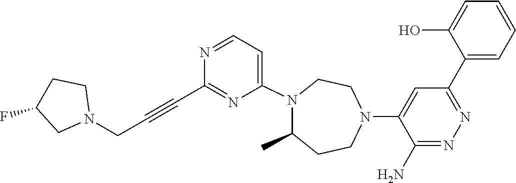 [SMILES: C[C@@H]1CCN(c2cc(-c3ccccc3O)nnc2N)CCN1c1ccnc(C#CCN2CC[C@@H](F)C2)n1]

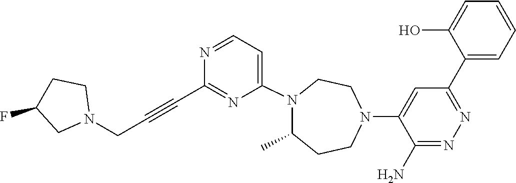 [SMILES: C[C@H]1CCN(c2cc(-c3ccccc3O)nnc2N)CCN1c1ccnc(C#CCN2CC[C@H](F)C2)n1]

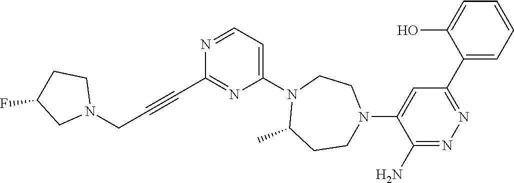 [SMILES: C[C@H]1CCN(c2cc(-c3ccccc3O)nnc2N)CCN1c1ccnc(C#CCN2CC[C@@H](F)C2)n1]

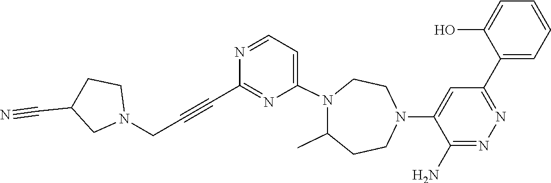 [SMILES: CC1CCN(c2cc(-c3ccccc3O)nnc2N)CCN1c1ccnc(C#CCN2CCC(C#N)C2)n1]

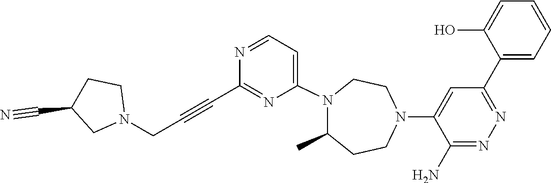 [SMILES: C[C@@H]1CCN(c2cc(-c3ccccc3O)nnc2N)CCN1c1ccnc(C#CCN2CC[C@H](C#N)C2)n1]

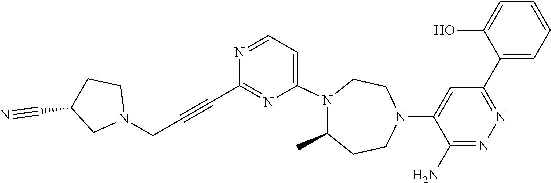 [SMILES: C[C@@H]1CCN(c2cc(-c3ccccc3O)nnc2N)CCN1c1ccnc(C#CCN2CC[C@@H](C#N)C2)n1]

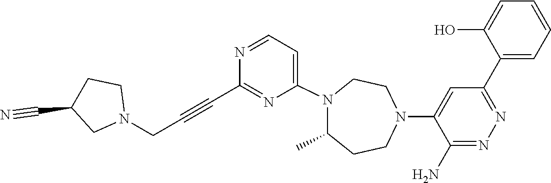 [SMILES: C[C@H]1CCN(c2cc(-c3ccccc3O)nnc2N)CCN1c1ccnc(C#CCN2CC[C@H](C#N)C2)n1]

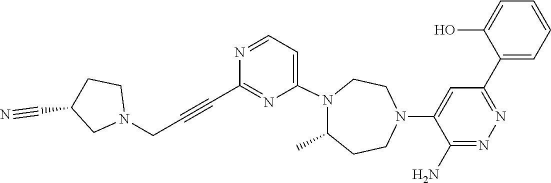 [SMILES: C[C@H]1CCN(c2cc(-c3ccccc3O)nnc2N)CCN1c1ccnc(C#CCN2CC[C@@H](C#N)C2)n1]

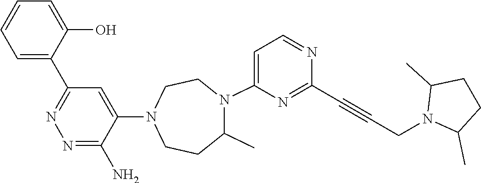 [SMILES: CC1CCN(c2cc(-c3ccccc3O)nnc2N)CCN1c1ccnc(C#CCN2C(C)CCC2C)n1]

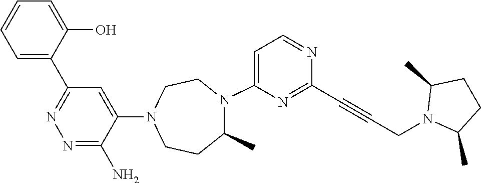 [SMILES: C[C@@H]1CC[C@H](C)N1CC#Cc1nccc(N2CCN(c3cc(-c4ccccc4O)nnc3N)CC[C@@H]2C)n1]

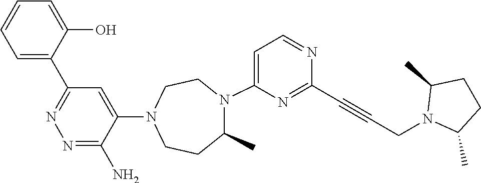 [SMILES: C[C@H]1CCN(c2cc(-c3ccccc3O)nnc2N)CCN1c1ccnc(C#CCN2[C@@H](C)CC[C@@H]2C)n1]

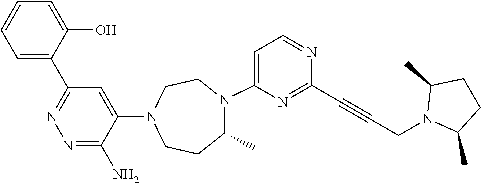 [SMILES: C[C@@H]1CCN(c2cc(-c3ccccc3O)nnc2N)CCN1c1ccnc(C#CCN2[C@H](C)CC[C@@H]2C)n1]

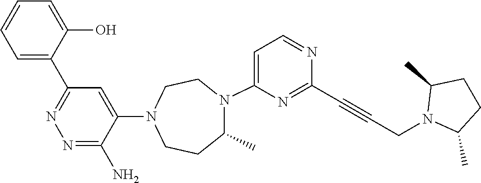 [SMILES: C[C@@H]1CCN(c2cc(-c3ccccc3O)nnc2N)CCN1c1ccnc(C#CCN2[C@@H](C)CC[C@@H]2C)n1]

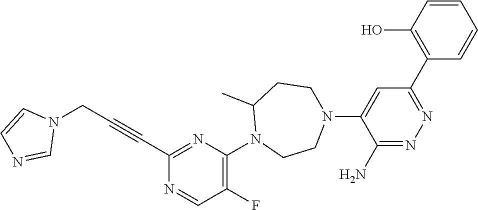 [SMILES: CC1CCN(c2cc(-c3ccccc3O)nnc2N)CCN1c1nc(C#CCn2ccnc2)ncc1F]

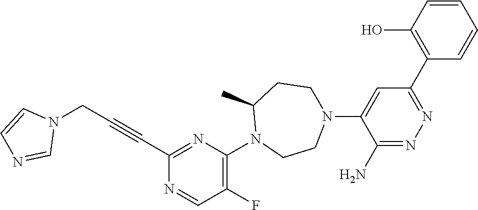 [SMILES: C[C@H]1CCN(c2cc(-c3ccccc3O)nnc2N)CCN1c1nc(C#CCn2ccnc2)ncc1F]

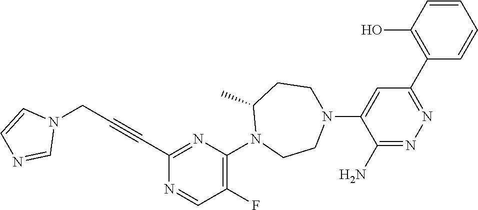 [SMILES: C[C@@H]1CCN(c2cc(-c3ccccc3O)nnc2N)CCN1c1nc(C#CCn2ccnc2)ncc1F]

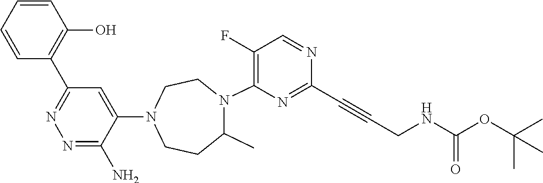 [SMILES: CC1CCN(c2cc(-c3ccccc3O)nnc2N)CCN1c1nc(C#CCNC(=O)OC(C)(C)C)ncc1F]

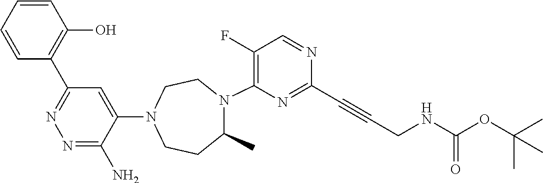 [SMILES: C[C@H]1CCN(c2cc(-c3ccccc3O)nnc2N)CCN1c1nc(C#CCNC(=O)OC(C)(C)C)ncc1F]

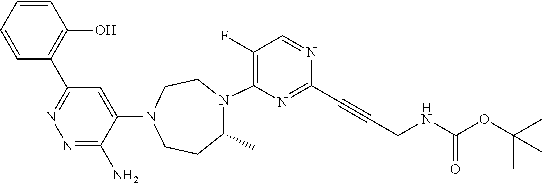 [SMILES: C[C@@H]1CCN(c2cc(-c3ccccc3O)nnc2N)CCN1c1nc(C#CCNC(=O)OC(C)(C)C)ncc1F]

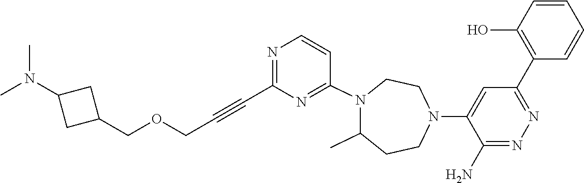 [SMILES: CC1CCN(c2cc(-c3ccccc3O)nnc2N)CCN1c1ccnc(C#CCOCC2CC(N(C)C)C2)n1]

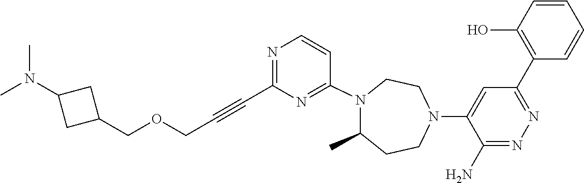 [SMILES: C[C@@H]1CCN(c2cc(-c3ccccc3O)nnc2N)CCN1c1ccnc(C#CCOCC2CC(N(C)C)C2)n1]

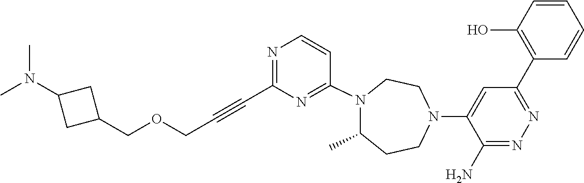 [SMILES: C[C@H]1CCN(c2cc(-c3ccccc3O)nnc2N)CCN1c1ccnc(C#CCOCC2CC(N(C)C)C2)n1]

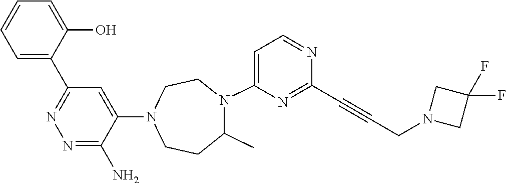 [SMILES: CC1CCN(c2cc(-c3ccccc3O)nnc2N)CCN1c1ccnc(C#CCN2CC(F)(F)C2)n1]

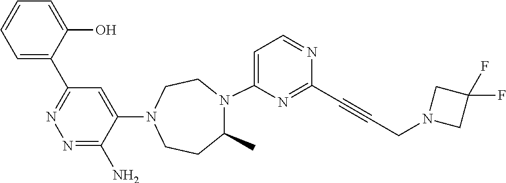 [SMILES: C[C@H]1CCN(c2cc(-c3ccccc3O)nnc2N)CCN1c1ccnc(C#CCN2CC(F)(F)C2)n1]

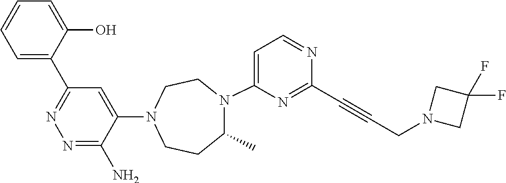 [SMILES: C[C@@H]1CCN(c2cc(-c3ccccc3O)nnc2N)CCN1c1ccnc(C#CCN2CC(F)(F)C2)n1]

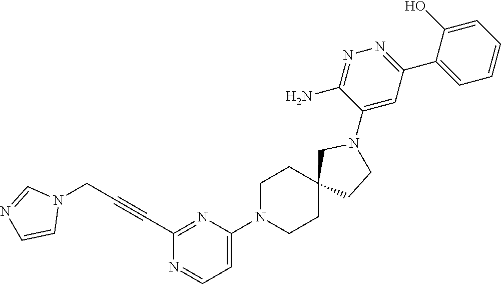

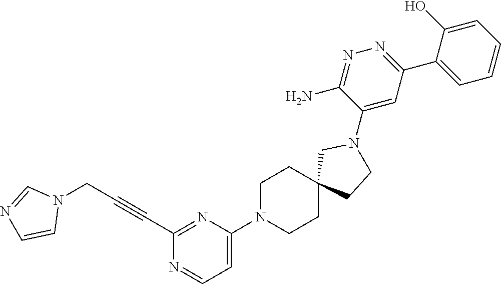

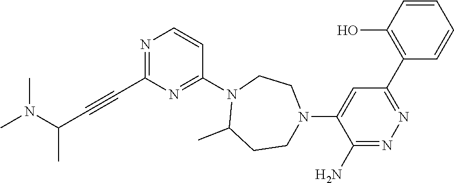 [SMILES: CC(C#Cc1nccc(N2CCN(c3cc(-c4ccccc4O)nnc3N)CCC2C)n1)N(C)C]

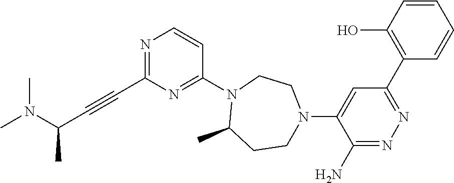 [SMILES: C[C@H](C#Cc1nccc(N2CCN(c3cc(-c4ccccc4O)nnc3N)CC[C@H]2C)n1)N(C)C]

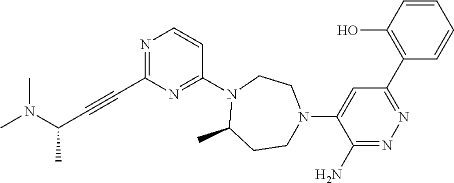 [SMILES: C[C@@H]1CCN(c2cc(-c3ccccc3O)nnc2N)CCN1c1ccnc(C#C[C@H](C)N(C)C)n1]

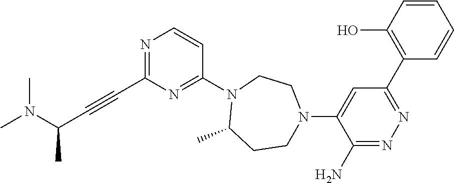 [SMILES: C[C@H](C#Cc1nccc(N2CCN(c3cc(-c4ccccc4O)nnc3N)CC[C@@H]2C)n1)N(C)C]

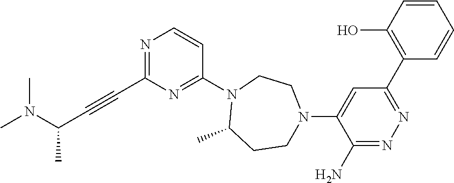 [SMILES: C[C@@H](C#Cc1nccc(N2CCN(c3cc(-c4ccccc4O)nnc3N)CC[C@@H]2C)n1)N(C)C]

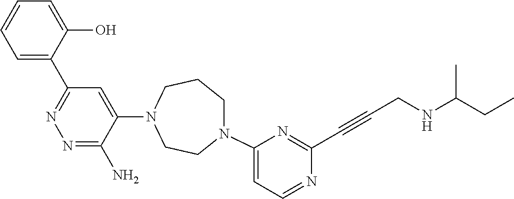 [SMILES: CCC(C)NCC#Cc1nccc(N2CCCN(c3cc(-c4ccccc4O)nnc3N)CC2)n1]

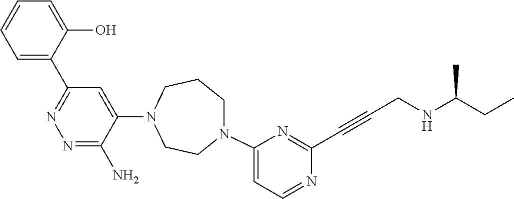 [SMILES: CC[C@H](C)NCC#Cc1nccc(N2CCCN(c3cc(-c4ccccc4O)nnc3N)CC2)n1]

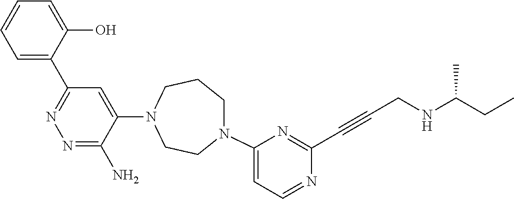 [SMILES: CC[C@@H](C)NCC#Cc1nccc(N2CCCN(c3cc(-c4ccccc4O)nnc3N)CC2)n1]

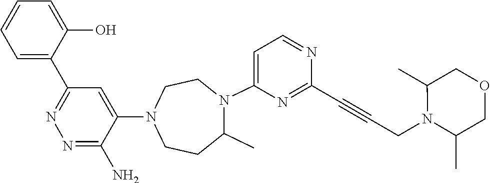 [SMILES: CC1CCN(c2cc(-c3ccccc3O)nnc2N)CCN1c1ccnc(C#CCN2C(C)COCC2C)n1]

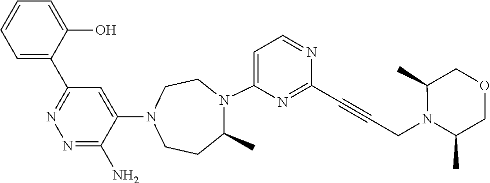 [SMILES: C[C@@H]1COC[C@H](C)N1CC#Cc1nccc(N2CCN(c3cc(-c4ccccc4O)nnc3N)CC[C@@H]2C)n1]

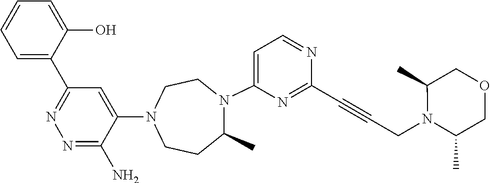 [SMILES: C[C@H]1CCN(c2cc(-c3ccccc3O)nnc2N)CCN1c1ccnc(C#CCN2[C@@H](C)COC[C@@H]2C)n1]

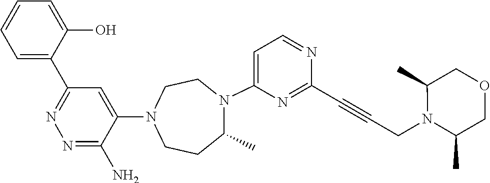 [SMILES: C[C@@H]1CCN(c2cc(-c3ccccc3O)nnc2N)CCN1c1ccnc(C#CCN2[C@H](C)COC[C@@H]2C)n1]

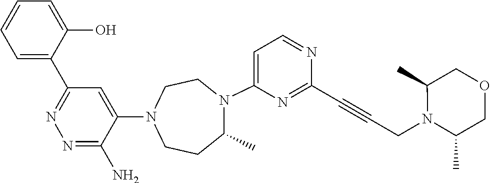 [SMILES: C[C@@H]1CCN(c2cc(-c3ccccc3O)nnc2N)CCN1c1ccnc(C#CCN2[C@@H](C)COC[C@@H]2C)n1]

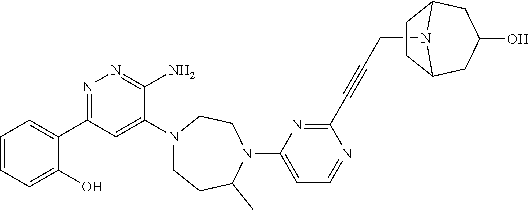 [SMILES: CC1CCN(c2cc(-c3ccccc3O)nnc2N)CCN1c1ccnc(C#CCN2C3CCC2CC(O)C3)n1]

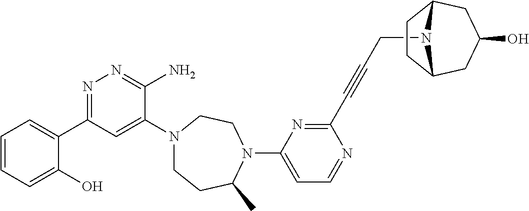 [SMILES: C[C@H]1CCN(c2cc(-c3ccccc3O)nnc2N)CCN1c1ccnc(C#CCN2[C@@H]3CC[C@H]2C[C@H](O)C3)n1]

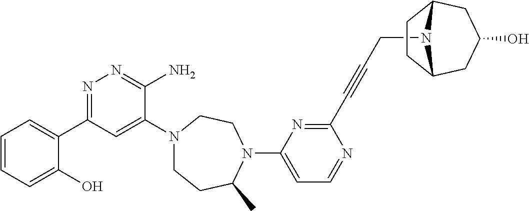 [SMILES: C[C@H]1CCN(c2cc(-c3ccccc3O)nnc2N)CCN1c1ccnc(C#CCN2[C@@H]3CC[C@H]2C[C@@H](O)C3)n1]

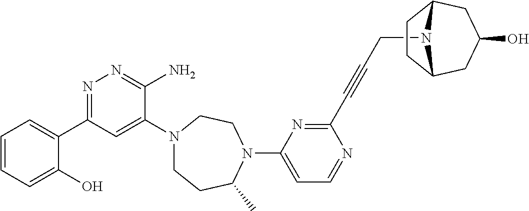 [SMILES: C[C@@H]1CCN(c2cc(-c3ccccc3O)nnc2N)CCN1c1ccnc(C#CCN2[C@@H]3CC[C@H]2C[C@H](O)C3)n1]

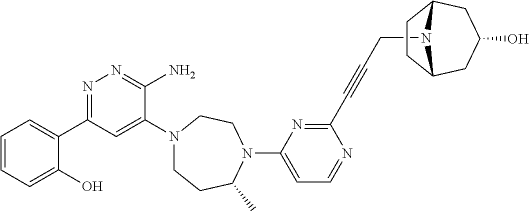 [SMILES: C[C@@H]1CCN(c2cc(-c3ccccc3O)nnc2N)CCN1c1ccnc(C#CCN2[C@@H]3CC[C@H]2C[C@@H](O)C3)n1]

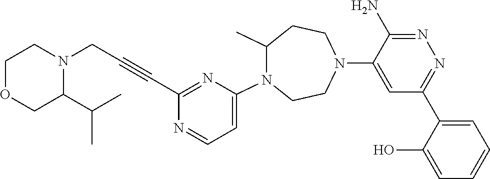 [SMILES: CC(C)C1COCCN1CC#Cc1nccc(N2CCN(c3cc(-c4ccccc4O)nnc3N)CCC2C)n1]

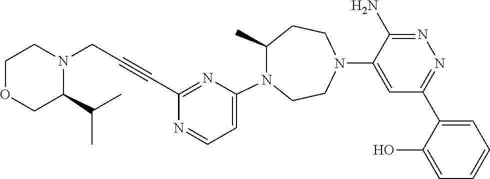 [SMILES: CC(C)[C@H]1COCCN1CC#Cc1nccc(N2CCN(c3cc(-c4ccccc4O)nnc3N)CC[C@@H]2C)n1]

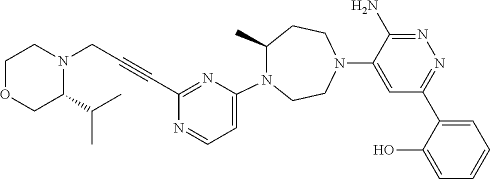 [SMILES: CC(C)[C@@H]1COCCN1CC#Cc1nccc(N2CCN(c3cc(-c4ccccc4O)nnc3N)CC[C@@H]2C)n1]

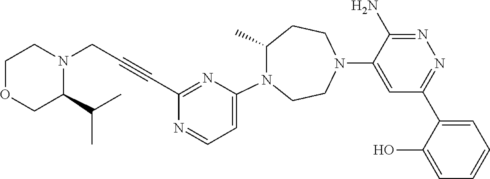 [SMILES: CC(C)[C@H]1COCCN1CC#Cc1nccc(N2CCN(c3cc(-c4ccccc4O)nnc3N)CC[C@H]2C)n1]

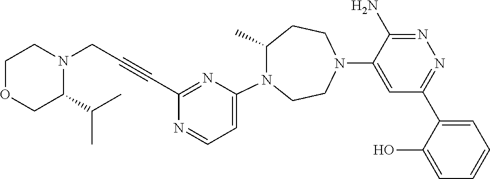 [SMILES: CC(C)[C@@H]1COCCN1CC#Cc1nccc(N2CCN(c3cc(-c4ccccc4O)nnc3N)CC[C@H]2C)n1]

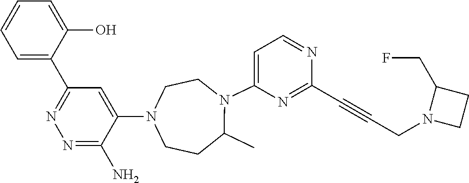 [SMILES: CC1CCN(c2cc(-c3ccccc3O)nnc2N)CCN1c1ccnc(C#CCN2CCC2CF)n1]

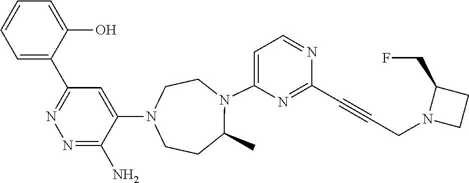 [SMILES: C[C@H]1CCN(c2cc(-c3ccccc3O)nnc2N)CCN1c1ccnc(C#CCN2CC[C@@H]2CF)n1]

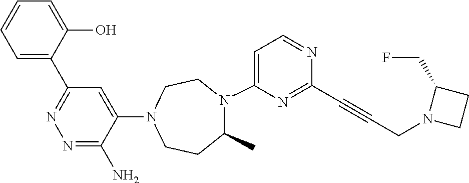 [SMILES: C[C@H]1CCN(c2cc(-c3ccccc3O)nnc2N)CCN1c1ccnc(C#CCN2CC[C@H]2CF)n1]

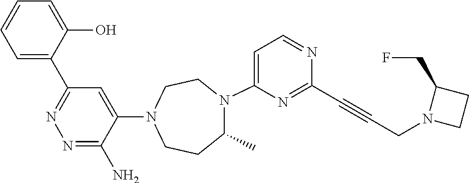 [SMILES: C[C@@H]1CCN(c2cc(-c3ccccc3O)nnc2N)CCN1c1ccnc(C#CCN2CC[C@@H]2CF)n1]

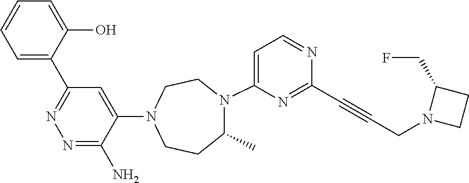 [SMILES: C[C@@H]1CCN(c2cc(-c3ccccc3O)nnc2N)CCN1c1ccnc(C#CCN2CC[C@H]2CF)n1]

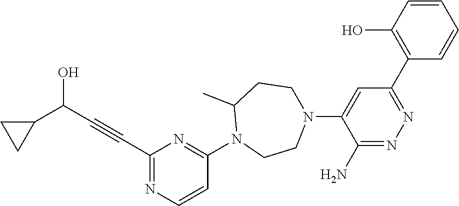 [SMILES: CC1CCN(c2cc(-c3ccccc3O)nnc2N)CCN1c1ccnc(C#CC(O)C2CC2)n1]

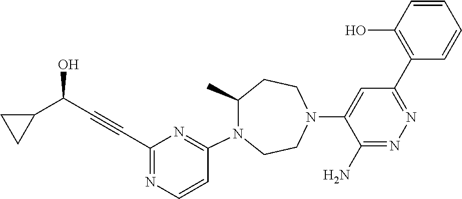 [SMILES: C[C@H]1CCN(c2cc(-c3ccccc3O)nnc2N)CCN1c1ccnc(C#C[C@H](O)C2CC2)n1]

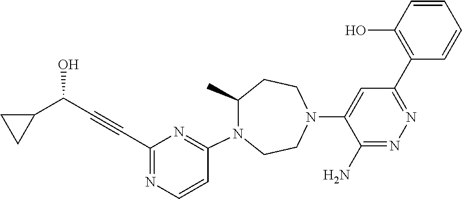 [SMILES: C[C@H]1CCN(c2cc(-c3ccccc3O)nnc2N)CCN1c1ccnc(C#C[C@@H](O)C2CC2)n1]

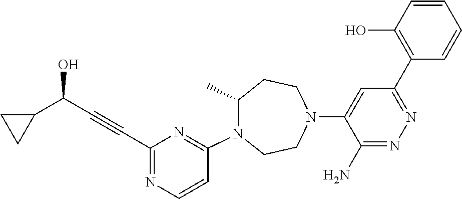 [SMILES: C[C@@H]1CCN(c2cc(-c3ccccc3O)nnc2N)CCN1c1ccnc(C#C[C@H](O)C2CC2)n1]

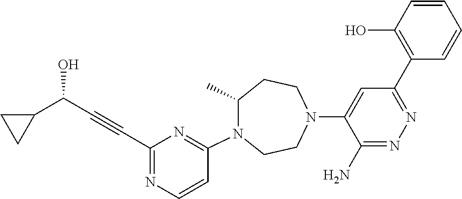 [SMILES: C[C@@H]1CCN(c2cc(-c3ccccc3O)nnc2N)CCN1c1ccnc(C#C[C@@H](O)C2CC2)n1]

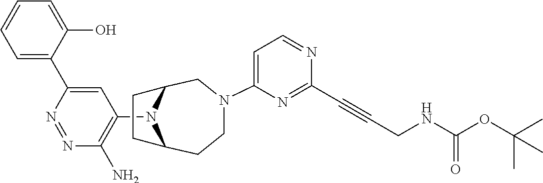 [SMILES: CC(C)(C)OC(=O)NCC#Cc1nccc(N2CC[C@@H]3CC[C@H](C2)N3c2cc(-c3ccccc3O)nnc2N)n1]

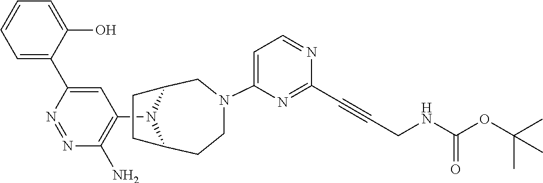 [SMILES: CC(C)(C)OC(=O)NCC#Cc1nccc(N2CC[C@H]3CC[C@@H](C2)N3c2cc(-c3ccccc3O)nnc2N)n1]

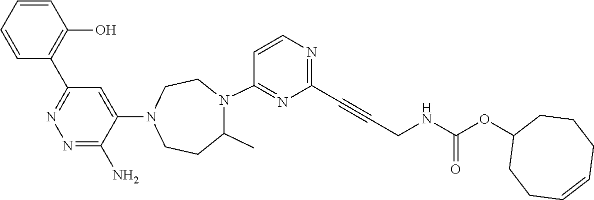 [SMILES: CC1CCN(c2cc(-c3ccccc3O)nnc2N)CCN1c1ccnc(C#CCNC(=O)OC2CC/C=C\CCC2)n1]

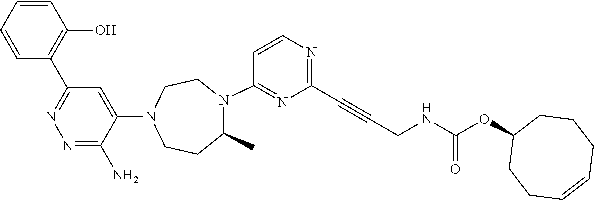 [SMILES: C[C@H]1CCN(c2cc(-c3ccccc3O)nnc2N)CCN1c1ccnc(C#CCNC(=O)O[C@@H]2CC/C=C\CCC2)n1]

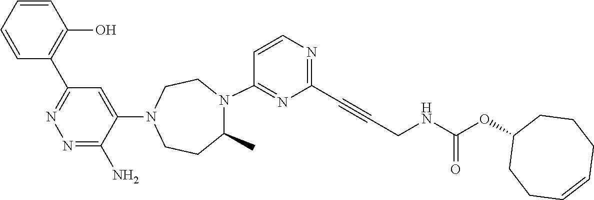 [SMILES: C[C@H]1CCN(c2cc(-c3ccccc3O)nnc2N)CCN1c1ccnc(C#CCNC(=O)O[C@H]2CC/C=C\CCC2)n1]

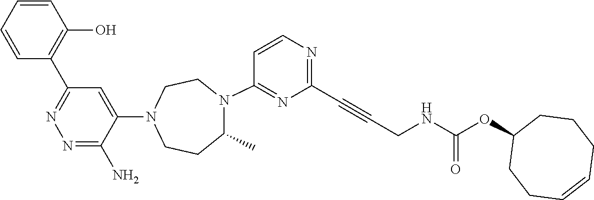 [SMILES: C[C@@H]1CCN(c2cc(-c3ccccc3O)nnc2N)CCN1c1ccnc(C#CCNC(=O)O[C@@H]2CC/C=C\CCC2)n1]

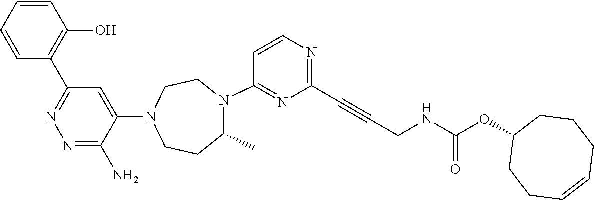 [SMILES: C[C@@H]1CCN(c2cc(-c3ccccc3O)nnc2N)CCN1c1ccnc(C#CCNC(=O)O[C@H]2CC/C=C\CCC2)n1]

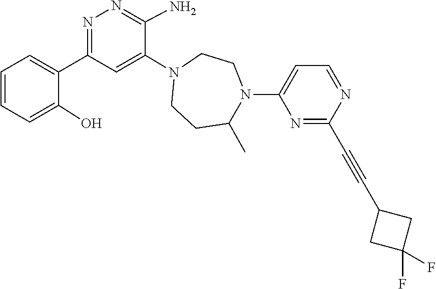 [SMILES: CC1CCN(c2cc(-c3ccccc3O)nnc2N)CCN1c1ccnc(C#CC2CC(F)(F)C2)n1]

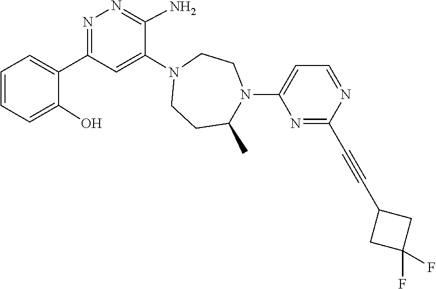 [SMILES: C[C@H]1CCN(c2cc(-c3ccccc3O)nnc2N)CCN1c1ccnc(C#CC2CC(F)(F)C2)n1]

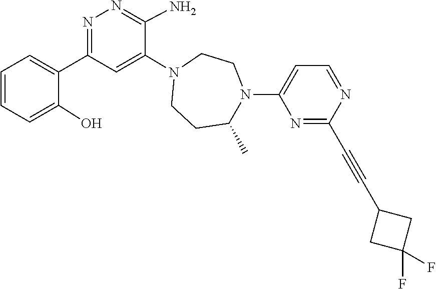 [SMILES: C[C@@H]1CCN(c2cc(-c3ccccc3O)nnc2N)CCN1c1ccnc(C#CC2CC(F)(F)C2)n1]

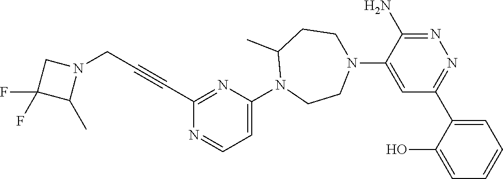 [SMILES: CC1CCN(c2cc(-c3ccccc3O)nnc2N)CCN1c1ccnc(C#CCN2CC(F)(F)C2C)n1]

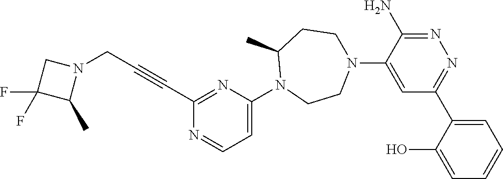 [SMILES: C[C@@H]1N(CC#Cc2nccc(N3CCN(c4cc(-c5ccccc5O)nnc4N)CC[C@@H]3C)n2)CC1(F)F]

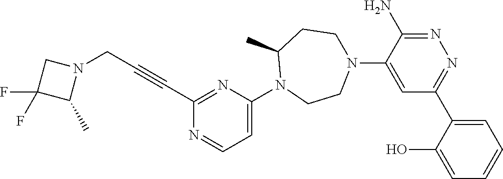 [SMILES: C[C@H]1CCN(c2cc(-c3ccccc3O)nnc2N)CCN1c1ccnc(C#CCN2CC(F)(F)[C@H]2C)n1]

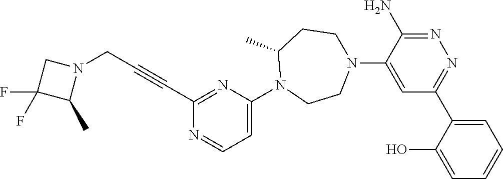 [SMILES: C[C@@H]1CCN(c2cc(-c3ccccc3O)nnc2N)CCN1c1ccnc(C#CCN2CC(F)(F)[C@@H]2C)n1]

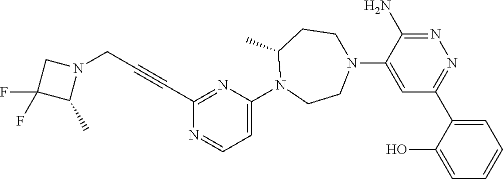 [SMILES: C[C@@H]1CCN(c2cc(-c3ccccc3O)nnc2N)CCN1c1ccnc(C#CCN2CC(F)(F)[C@H]2C)n1]

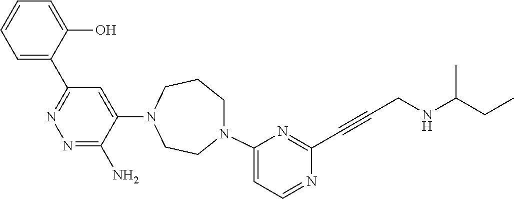 [SMILES: CCC(C)NCC#Cc1nccc(N2CCCN(c3cc(-c4ccccc4O)nnc3N)CC2)n1]

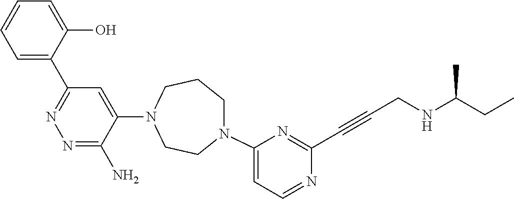 [SMILES: CC[C@H](C)NCC#Cc1nccc(N2CCCN(c3cc(-c4ccccc4O)nnc3N)CC2)n1]

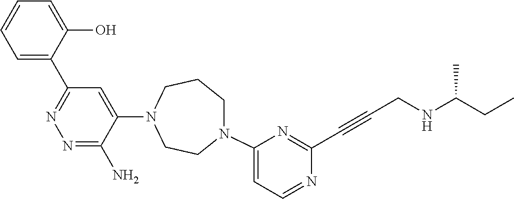 [SMILES: CC[C@@H](C)NCC#Cc1nccc(N2CCCN(c3cc(-c4ccccc4O)nnc3N)CC2)n1]

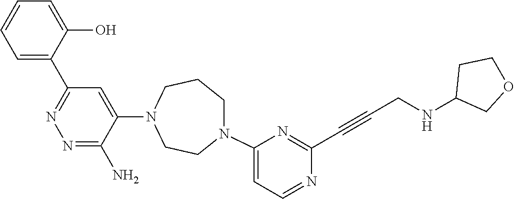 [SMILES: Nc1nnc(-c2ccccc2O)cc1N1CCCN(c2ccnc(C#CCNC3CCOC3)n2)CC1]

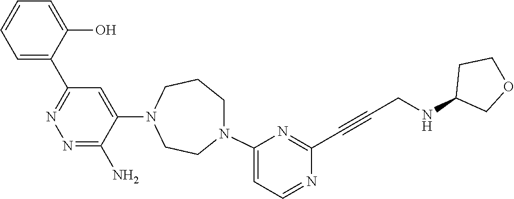 [SMILES: Nc1nnc(-c2ccccc2O)cc1N1CCCN(c2ccnc(C#CCN[C@H]3CCOC3)n2)CC1]

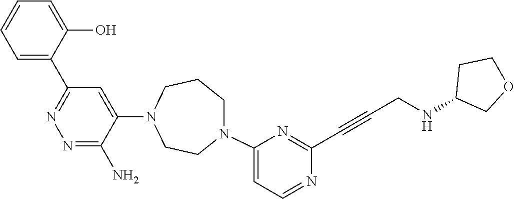 [SMILES: Nc1nnc(-c2ccccc2O)cc1N1CCCN(c2ccnc(C#CCN[C@@H]3CCOC3)n2)CC1]

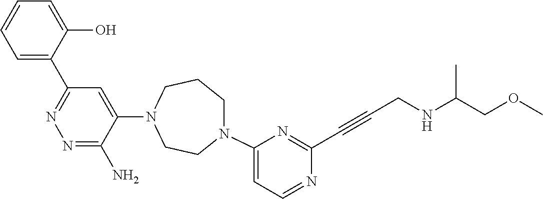 [SMILES: COCC(C)NCC#Cc1nccc(N2CCCN(c3cc(-c4ccccc4O)nnc3N)CC2)n1]

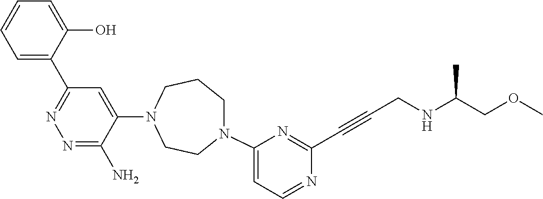 [SMILES: COC[C@H](C)NCC#Cc1nccc(N2CCCN(c3cc(-c4ccccc4O)nnc3N)CC2)n1]

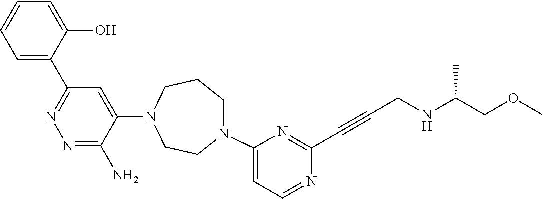 [SMILES: COC[C@@H](C)NCC#Cc1nccc(N2CCCN(c3cc(-c4ccccc4O)nnc3N)CC2)n1]

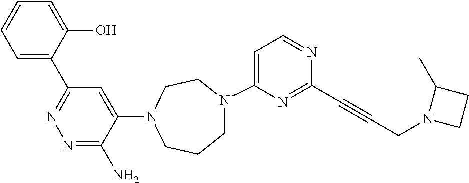 [SMILES: CC1CCN1CC#Cc1nccc(N2CCCN(c3cc(-c4ccccc4O)nnc3N)CC2)n1]

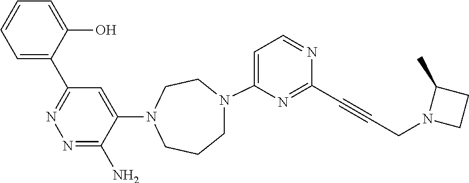 [SMILES: C[C@H]1CCN1CC#Cc1nccc(N2CCCN(c3cc(-c4ccccc4O)nnc3N)CC2)n1]

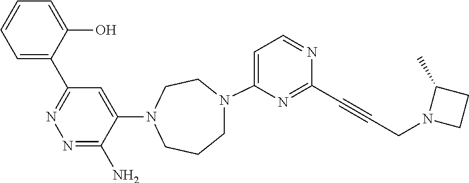 [SMILES: C[C@@H]1CCN1CC#Cc1nccc(N2CCCN(c3cc(-c4ccccc4O)nnc3N)CC2)n1]

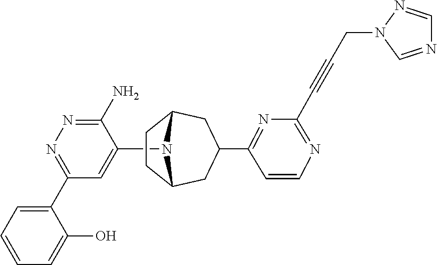 [SMILES: Nc1nnc(-c2ccccc2O)cc1N1[C@@H]2CC[C@H]1CC(c1ccnc(C#CCn3cncn3)n1)C2]

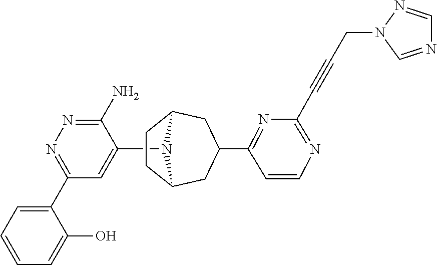 [SMILES: Nc1nnc(-c2ccccc2O)cc1N1[C@@H]2CC[C@H]1CC(c1ccnc(C#CCn3cncn3)n1)C2]

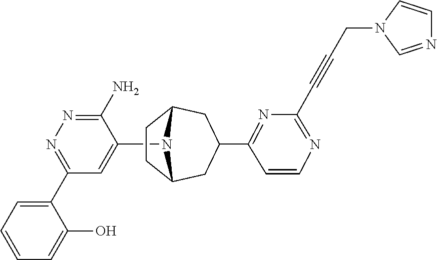 [SMILES: Nc1nnc(-c2ccccc2O)cc1N1[C@@H]2CC[C@H]1CC(c1ccnc(C#CCn3ccnc3)n1)C2]

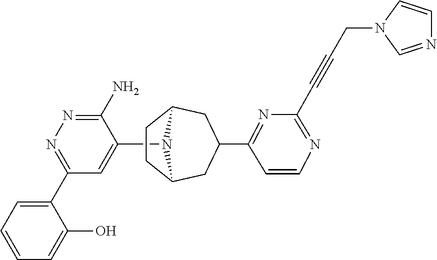 [SMILES: Nc1nnc(-c2ccccc2O)cc1N1[C@@H]2CC[C@H]1CC(c1ccnc(C#CCn3ccnc3)n1)C2]

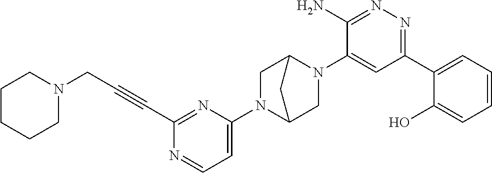 [SMILES: Nc1nnc(-c2ccccc2O)cc1N1CC2CC1CN2c1ccnc(C#CCN2CCCCC2)n1]

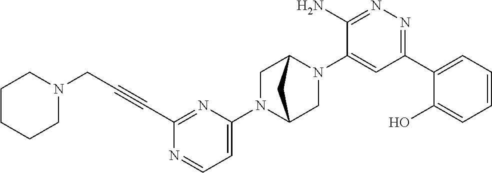 [SMILES: Nc1nnc(-c2ccccc2O)cc1N1C[C@H]2C[C@@H]1CN2c1ccnc(C#CCN2CCCCC2)n1]

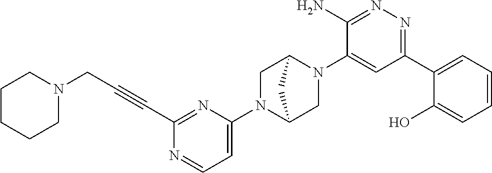 [SMILES: Nc1nnc(-c2ccccc2O)cc1N1C[C@@H]2C[C@H]1CN2c1ccnc(C#CCN2CCCCC2)n1]

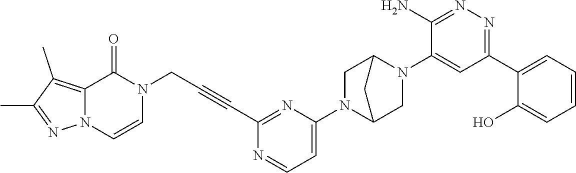 [SMILES: Cc1nn2ccn(CC#Cc3nccc(N4CC5CC4CN5c4cc(-c5ccccc5O)nnc4N)n3)c(=O)c2c1C]

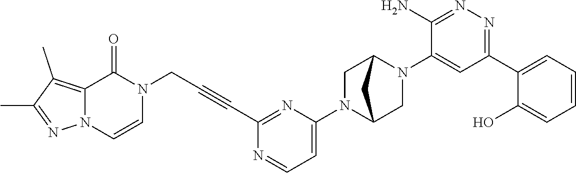 [SMILES: Cc1nn2ccn(CC#Cc3nccc(N4C[C@H]5C[C@@H]4CN5c4cc(-c5ccccc5O)nnc4N)n3)c(=O)c2c1C]

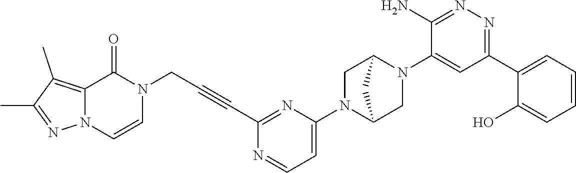 [SMILES: Cc1nn2ccn(CC#Cc3nccc(N4C[C@@H]5C[C@H]4CN5c4cc(-c5ccccc5O)nnc4N)n3)c(=O)c2c1C]

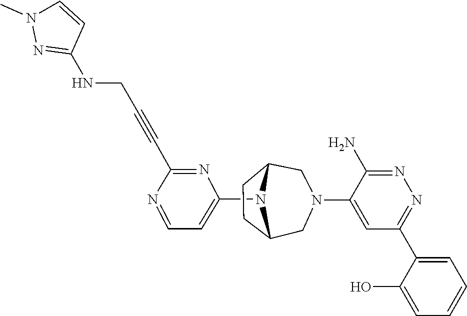 [SMILES: Cn1ccc(NCC#Cc2nccc(N3[C@@H]4CC[C@H]3CN(c3cc(-c5ccccc5O)nnc3N)C4)n2)n1]

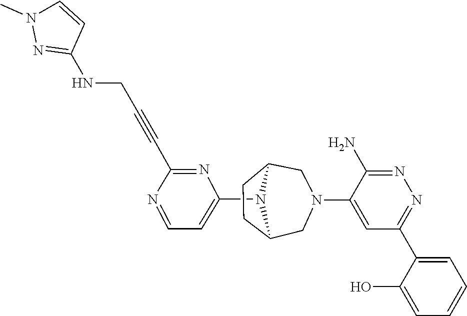 [SMILES: Cn1ccc(NCC#Cc2nccc(N3[C@@H]4CC[C@H]3CN(c3cc(-c5ccccc5O)nnc3N)C4)n2)n1]

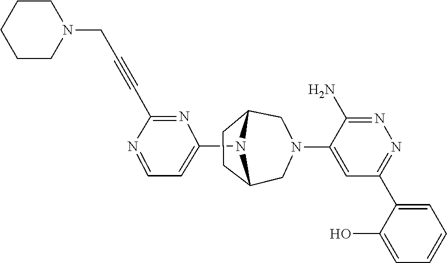 [SMILES: Nc1nnc(-c2ccccc2O)cc1N1C[C@H]2CC[C@@H](C1)N2c1ccnc(C#CCN2CCCCC2)n1]

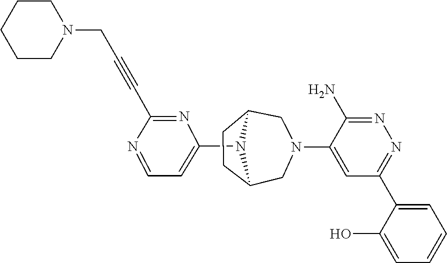 [SMILES: Nc1nnc(-c2ccccc2O)cc1N1C[C@H]2CC[C@@H](C1)N2c1ccnc(C#CCN2CCCCC2)n1]

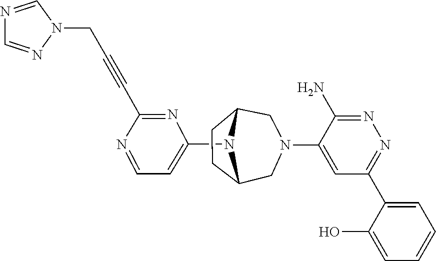 [SMILES: Nc1nnc(-c2ccccc2O)cc1N1C[C@H]2CC[C@@H](C1)N2c1ccnc(C#CCn2cncn2)n1]

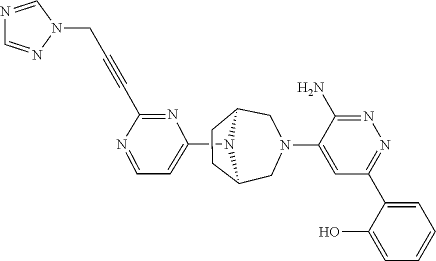 [SMILES: Nc1nnc(-c2ccccc2O)cc1N1C[C@H]2CC[C@@H](C1)N2c1ccnc(C#CCn2cncn2)n1]

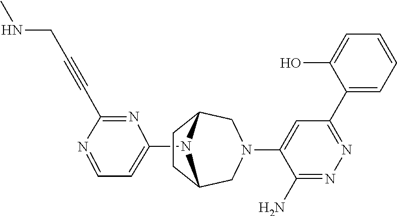 [SMILES: CNCC#Cc1nccc(N2[C@@H]3CC[C@H]2CN(c2cc(-c4ccccc4O)nnc2N)C3)n1]

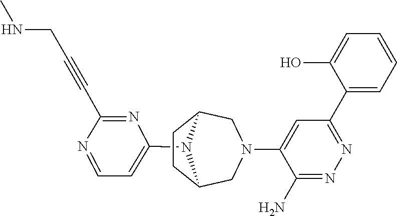 [SMILES: CNCC#Cc1nccc(N2[C@@H]3CC[C@H]2CN(c2cc(-c4ccccc4O)nnc2N)C3)n1]

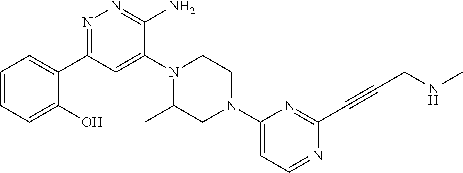 [SMILES: CNCC#Cc1nccc(N2CCN(c3cc(-c4ccccc4O)nnc3N)C(C)C2)n1]

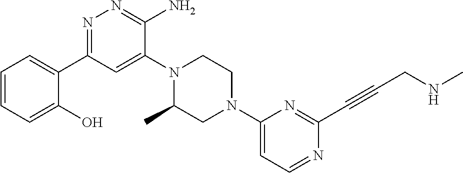 [SMILES: CNCC#Cc1nccc(N2CCN(c3cc(-c4ccccc4O)nnc3N)[C@H](C)C2)n1]

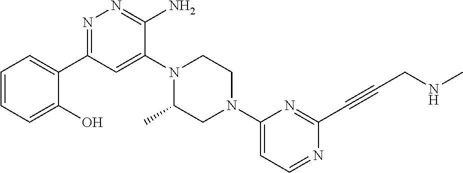 [SMILES: CNCC#Cc1nccc(N2CCN(c3cc(-c4ccccc4O)nnc3N)[C@@H](C)C2)n1]

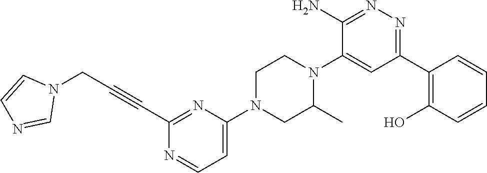 [SMILES: CC1CN(c2ccnc(C#CCn3ccnc3)n2)CCN1c1cc(-c2ccccc2O)nnc1N]

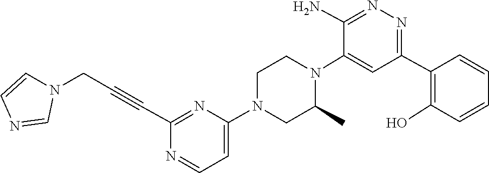 [SMILES: C[C@H]1CN(c2ccnc(C#CCn3ccnc3)n2)CCN1c1cc(-c2ccccc2O)nnc1N]

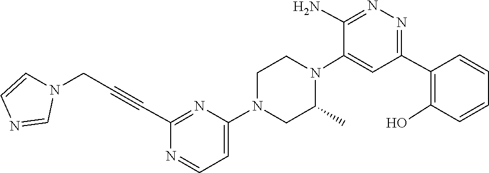 [SMILES: C[C@@H]1CN(c2ccnc(C#CCn3ccnc3)n2)CCN1c1cc(-c2ccccc2O)nnc1N]

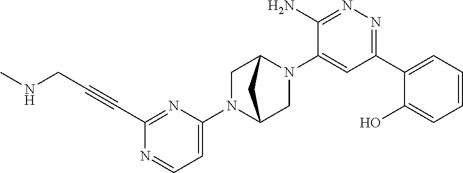 [SMILES: CNCC#Cc1nccc(N2C[C@H]3C[C@@H]2CN3c2cc(-c3ccccc3O)nnc2N)n1]

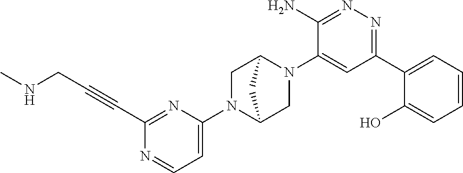 [SMILES: CNCC#Cc1nccc(N2C[C@@H]3C[C@H]2CN3c2cc(-c3ccccc3O)nnc2N)n1]

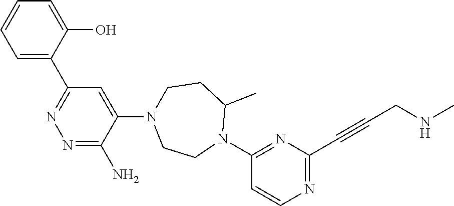 [SMILES: CNCC#Cc1nccc(N2CCN(c3cc(-c4ccccc4O)nnc3N)CCC2C)n1]

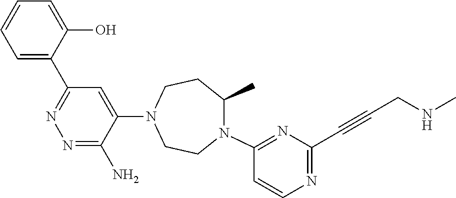 [SMILES: CNCC#Cc1nccc(N2CCN(c3cc(-c4ccccc4O)nnc3N)CC[C@H]2C)n1]

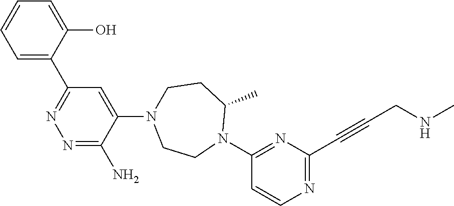 [SMILES: CNCC#Cc1nccc(N2CCN(c3cc(-c4ccccc4O)nnc3N)CC[C@@H]2C)n1]

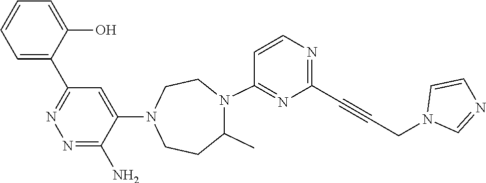 [SMILES: CC1CCN(c2cc(-c3ccccc3O)nnc2N)CCN1c1ccnc(C#CCn2ccnc2)n1]

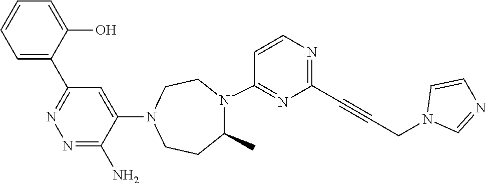 [SMILES: C[C@H]1CCN(c2cc(-c3ccccc3O)nnc2N)CCN1c1ccnc(C#CCn2ccnc2)n1]

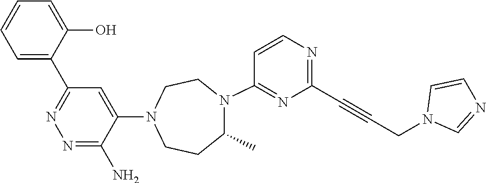 [SMILES: C[C@@H]1CCN(c2cc(-c3ccccc3O)nnc2N)CCN1c1ccnc(C#CCn2ccnc2)n1]

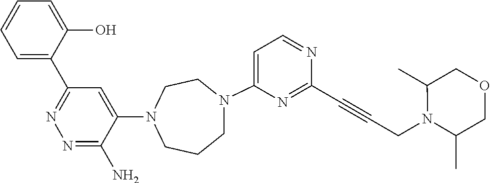 [SMILES: CC1COCC(C)N1CC#Cc1nccc(N2CCCN(c3cc(-c4ccccc4O)nnc3N)CC2)n1]

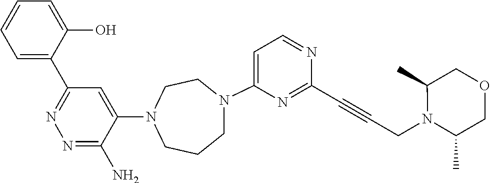 [SMILES: C[C@H]1COC[C@H](C)N1CC#Cc1nccc(N2CCCN(c3cc(-c4ccccc4O)nnc3N)CC2)n1]

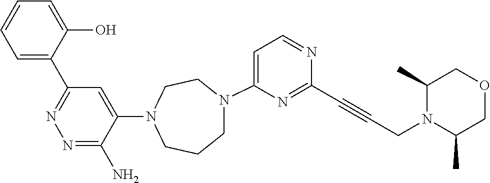 [SMILES: C[C@@H]1COC[C@H](C)N1CC#Cc1nccc(N2CCCN(c3cc(-c4ccccc4O)nnc3N)CC2)n1]

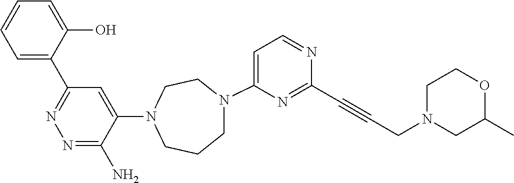 [SMILES: CC1CN(CC#Cc2nccc(N3CCCN(c4cc(-c5ccccc5O)nnc4N)CC3)n2)CCO1]

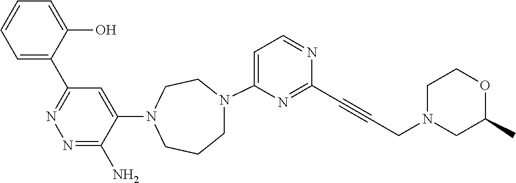 [SMILES: C[C@H]1CN(CC#Cc2nccc(N3CCCN(c4cc(-c5ccccc5O)nnc4N)CC3)n2)CCO1]

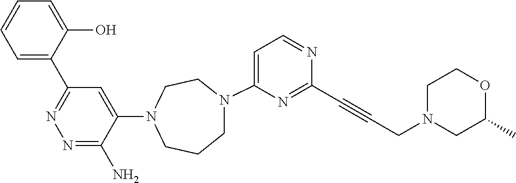 [SMILES: C[C@@H]1CN(CC#Cc2nccc(N3CCCN(c4cc(-c5ccccc5O)nnc4N)CC3)n2)CCO1]

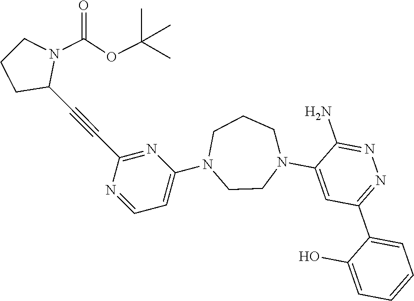 [SMILES: CC(C)(C)OC(=O)N1CCCC1C#Cc1nccc(N2CCCN(c3cc(-c4ccccc4O)nnc3N)CC2)n1]

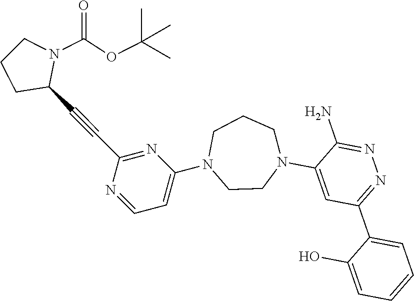 [SMILES: CC(C)(C)OC(=O)N1CCC[C@@H]1C#Cc1nccc(N2CCCN(c3cc(-c4ccccc4O)nnc3N)CC2)n1]

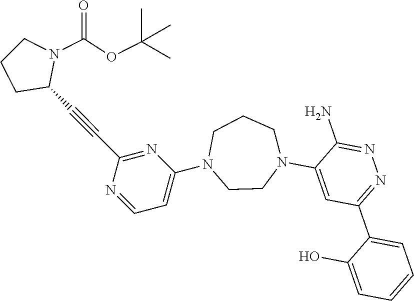 [SMILES: CC(C)(C)OC(=O)N1CCC[C@H]1C#Cc1nccc(N2CCCN(c3cc(-c4ccccc4O)nnc3N)CC2)n1]

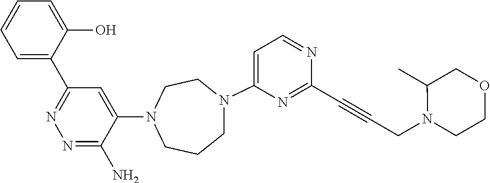 [SMILES: CC1COCCN1CC#Cc1nccc(N2CCCN(c3cc(-c4ccccc4O)nnc3N)CC2)n1]

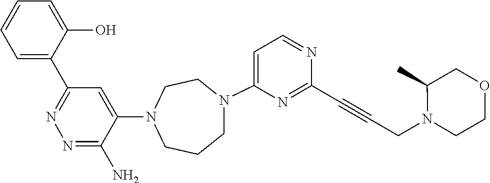 [SMILES: C[C@H]1COCCN1CC#Cc1nccc(N2CCCN(c3cc(-c4ccccc4O)nnc3N)CC2)n1]

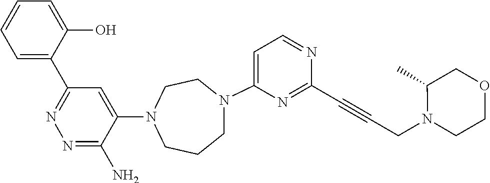 [SMILES: C[C@@H]1COCCN1CC#Cc1nccc(N2CCCN(c3cc(-c4ccccc4O)nnc3N)CC2)n1]

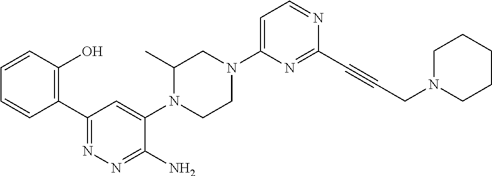 [SMILES: CC1CN(c2ccnc(C#CCN3CCCCC3)n2)CCN1c1cc(-c2ccccc2O)nnc1N]

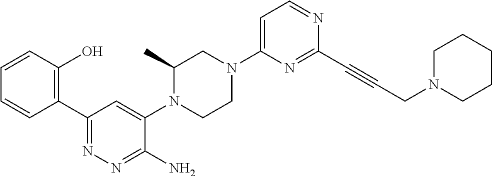 [SMILES: C[C@H]1CN(c2ccnc(C#CCN3CCCCC3)n2)CCN1c1cc(-c2ccccc2O)nnc1N]

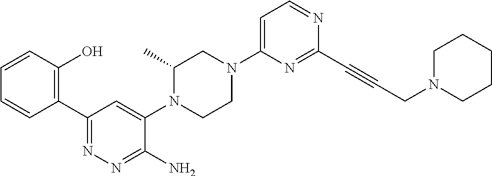 [SMILES: C[C@@H]1CN(c2ccnc(C#CCN3CCCCC3)n2)CCN1c1cc(-c2ccccc2O)nnc1N]

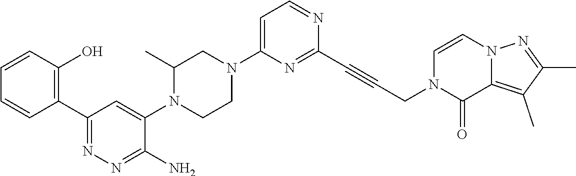 [SMILES: Cc1nn2ccn(CC#Cc3nccc(N4CCN(c5cc(-c6ccccc6O)nnc5N)C(C)C4)n3)c(=O)c2c1C]

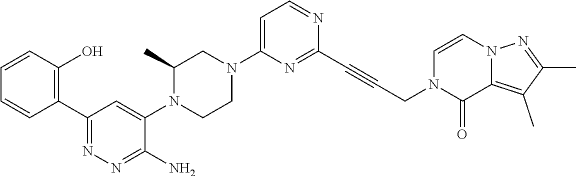 [SMILES: Cc1nn2ccn(CC#Cc3nccc(N4CCN(c5cc(-c6ccccc6O)nnc5N)[C@@H](C)C4)n3)c(=O)c2c1C]

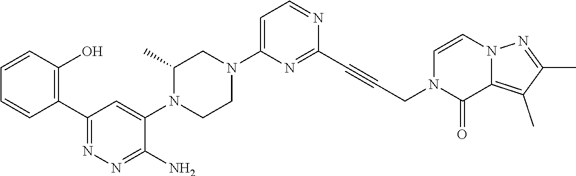 [SMILES: Cc1nn2ccn(CC#Cc3nccc(N4CCN(c5cc(-c6ccccc6O)nnc5N)[C@H](C)C4)n3)c(=O)c2c1C]

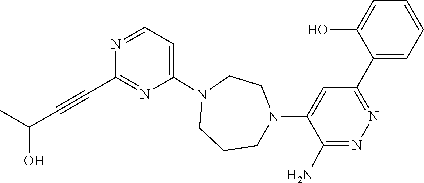 [SMILES: CC(O)C#Cc1nccc(N2CCCN(c3cc(-c4ccccc4O)nnc3N)CC2)n1]

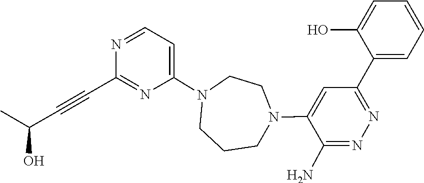 [SMILES: C[C@H](O)C#Cc1nccc(N2CCCN(c3cc(-c4ccccc4O)nnc3N)CC2)n1]

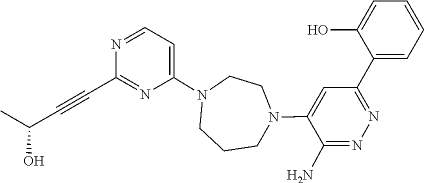 [SMILES: C[C@@H](O)C#Cc1nccc(N2CCCN(c3cc(-c4ccccc4O)nnc3N)CC2)n1]

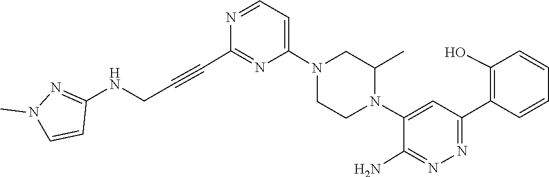 [SMILES: CC1CN(c2ccnc(C#CCNc3ccn(C)n3)n2)CCN1c1cc(-c2ccccc2O)nnc1N]

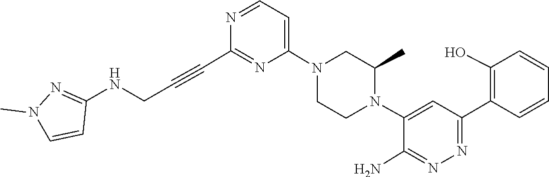 [SMILES: C[C@@H]1CN(c2ccnc(C#CCNc3ccn(C)n3)n2)CCN1c1cc(-c2ccccc2O)nnc1N]

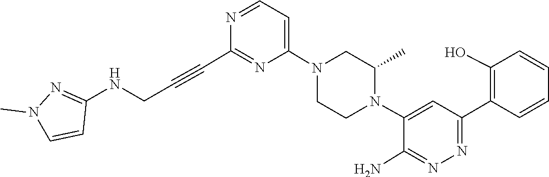 [SMILES: C[C@H]1CN(c2ccnc(C#CCNc3ccn(C)n3)n2)CCN1c1cc(-c2ccccc2O)nnc1N]

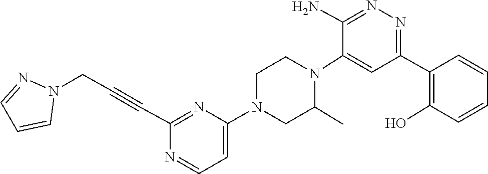 [SMILES: CC1CN(c2ccnc(C#CCn3cccn3)n2)CCN1c1cc(-c2ccccc2O)nnc1N]

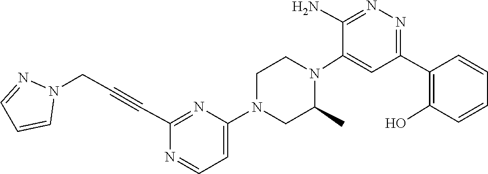 [SMILES: C[C@H]1CN(c2ccnc(C#CCn3cccn3)n2)CCN1c1cc(-c2ccccc2O)nnc1N]

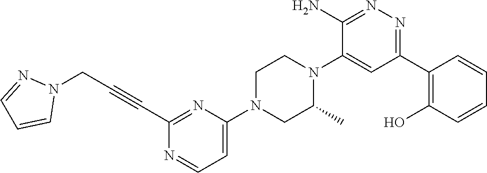 [SMILES: C[C@@H]1CN(c2ccnc(C#CCn3cccn3)n2)CCN1c1cc(-c2ccccc2O)nnc1N]

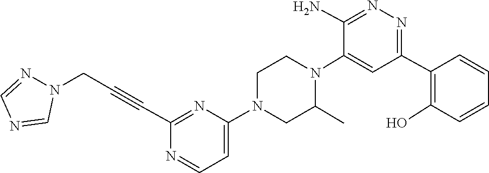 [SMILES: CC1CN(c2ccnc(C#CCn3cncn3)n2)CCN1c1cc(-c2ccccc2O)nnc1N]

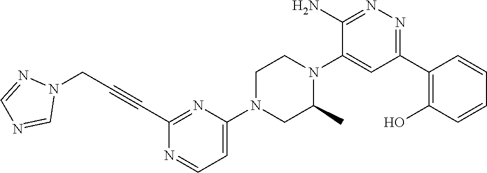 [SMILES: C[C@H]1CN(c2ccnc(C#CCn3cncn3)n2)CCN1c1cc(-c2ccccc2O)nnc1N]

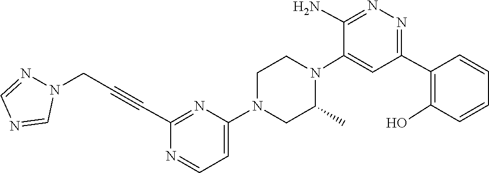 [SMILES: C[C@@H]1CN(c2ccnc(C#CCn3cncn3)n2)CCN1c1cc(-c2ccccc2O)nnc1N]

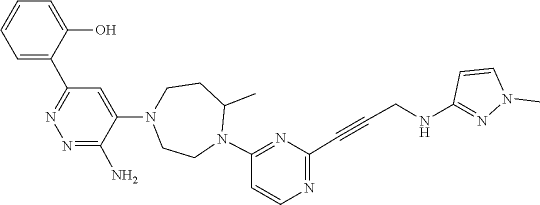 [SMILES: CC1CCN(c2cc(-c3ccccc3O)nnc2N)CCN1c1ccnc(C#CCNc2ccn(C)n2)n1]

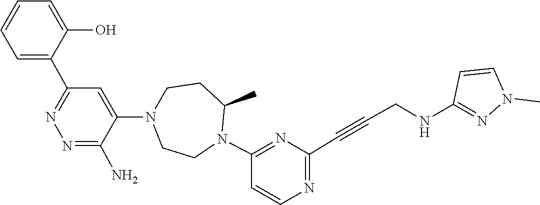 [SMILES: C[C@@H]1CCN(c2cc(-c3ccccc3O)nnc2N)CCN1c1ccnc(C#CCNc2ccn(C)n2)n1]

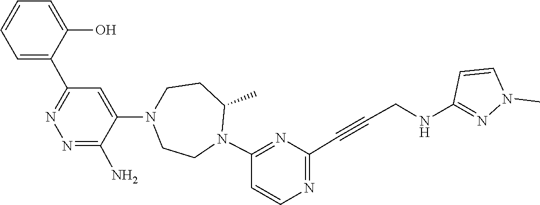 [SMILES: C[C@H]1CCN(c2cc(-c3ccccc3O)nnc2N)CCN1c1ccnc(C#CCNc2ccn(C)n2)n1]

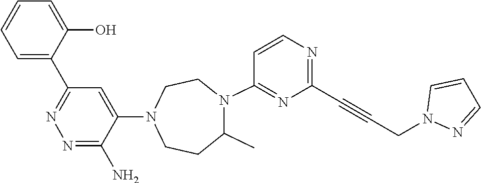 [SMILES: CC1CCN(c2cc(-c3ccccc3O)nnc2N)CCN1c1ccnc(C#CCn2cccn2)n1]

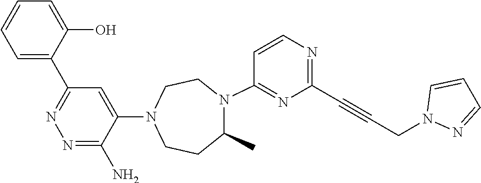 [SMILES: C[C@H]1CCN(c2cc(-c3ccccc3O)nnc2N)CCN1c1ccnc(C#CCn2cccn2)n1]

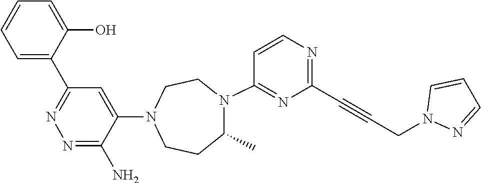 [SMILES: C[C@@H]1CCN(c2cc(-c3ccccc3O)nnc2N)CCN1c1ccnc(C#CCn2cccn2)n1]

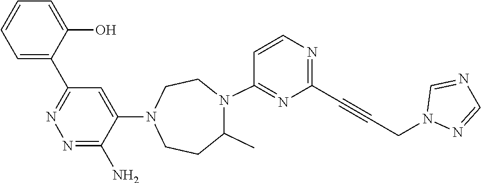 [SMILES: CC1CCN(c2cc(-c3ccccc3O)nnc2N)CCN1c1ccnc(C#CCn2cncn2)n1]

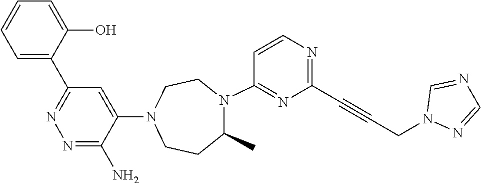 [SMILES: C[C@H]1CCN(c2cc(-c3ccccc3O)nnc2N)CCN1c1ccnc(C#CCn2cncn2)n1]

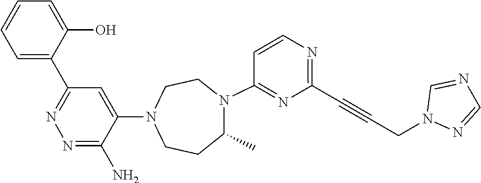 [SMILES: C[C@@H]1CCN(c2cc(-c3ccccc3O)nnc2N)CCN1c1ccnc(C#CCn2cncn2)n1]

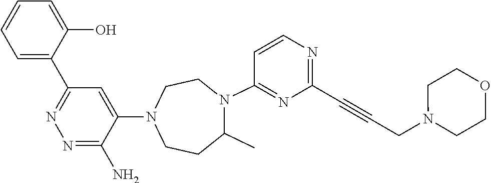 [SMILES: CC1CCN(c2cc(-c3ccccc3O)nnc2N)CCN1c1ccnc(C#CCN2CCOCC2)n1]

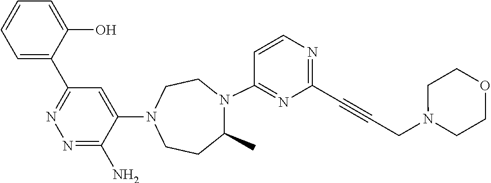 [SMILES: C[C@H]1CCN(c2cc(-c3ccccc3O)nnc2N)CCN1c1ccnc(C#CCN2CCOCC2)n1]

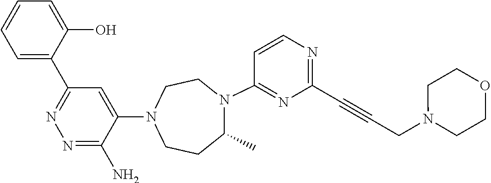 [SMILES: C[C@@H]1CCN(c2cc(-c3ccccc3O)nnc2N)CCN1c1ccnc(C#CCN2CCOCC2)n1]

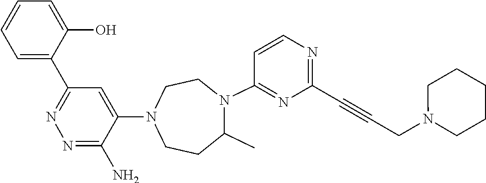 [SMILES: CC1CCN(c2cc(-c3ccccc3O)nnc2N)CCN1c1ccnc(C#CCN2CCCCC2)n1]

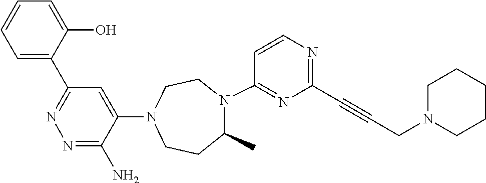 [SMILES: C[C@H]1CCN(c2cc(-c3ccccc3O)nnc2N)CCN1c1ccnc(C#CCN2CCCCC2)n1]

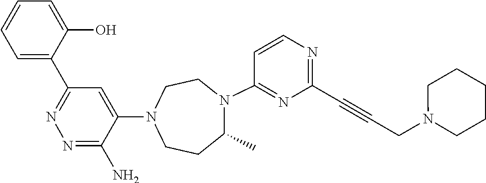 [SMILES: C[C@@H]1CCN(c2cc(-c3ccccc3O)nnc2N)CCN1c1ccnc(C#CCN2CCCCC2)n1]

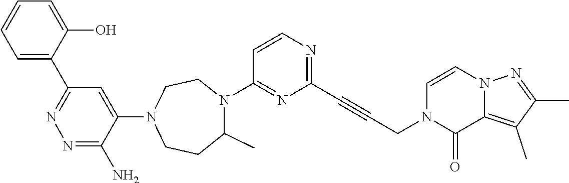 [SMILES: Cc1nn2ccn(CC#Cc3nccc(N4CCN(c5cc(-c6ccccc6O)nnc5N)CCC4C)n3)c(=O)c2c1C]

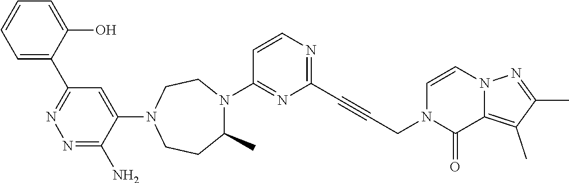 [SMILES: Cc1nn2ccn(CC#Cc3nccc(N4CCN(c5cc(-c6ccccc6O)nnc5N)CC[C@@H]4C)n3)c(=O)c2c1C]

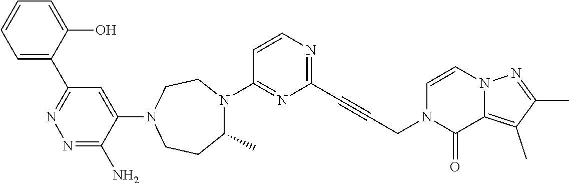 [SMILES: Cc1nn2ccn(CC#Cc3nccc(N4CCN(c5cc(-c6ccccc6O)nnc5N)CC[C@H]4C)n3)c(=O)c2c1C]

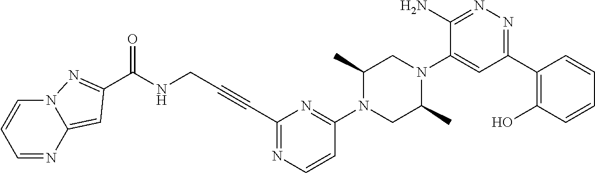 [SMILES: C[C@H]1CN(c2cc(-c3ccccc3O)nnc2N)[C@@H](C)CN1c1ccnc(C#CCNC(=O)c2cc3ncccn3n2)n1]

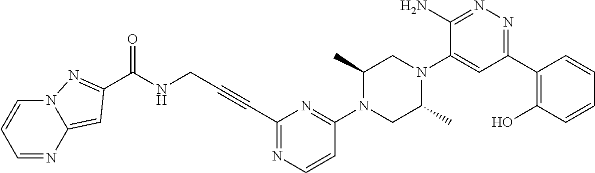 [SMILES: C[C@@H]1CN(c2ccnc(C#CCNC(=O)c3cc4ncccn4n3)n2)[C@@H](C)CN1c1cc(-c2ccccc2O)nnc1N]

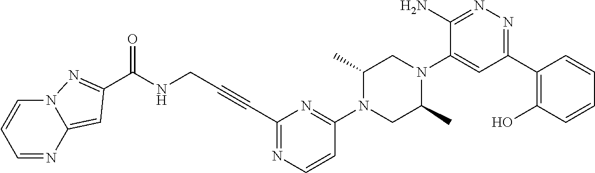 [SMILES: C[C@@H]1CN(c2cc(-c3ccccc3O)nnc2N)[C@@H](C)CN1c1ccnc(C#CCNC(=O)c2cc3ncccn3n2)n1]

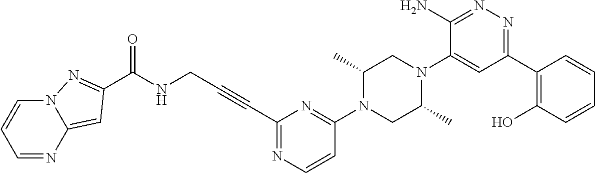 [SMILES: C[C@@H]1CN(c2cc(-c3ccccc3O)nnc2N)[C@H](C)CN1c1ccnc(C#CCNC(=O)c2cc3ncccn3n2)n1]

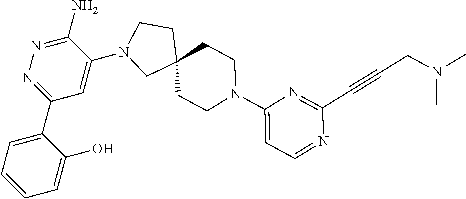

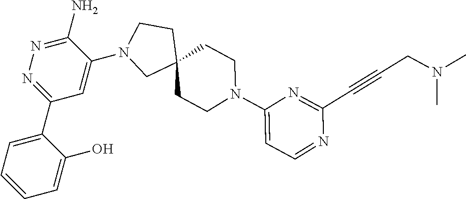

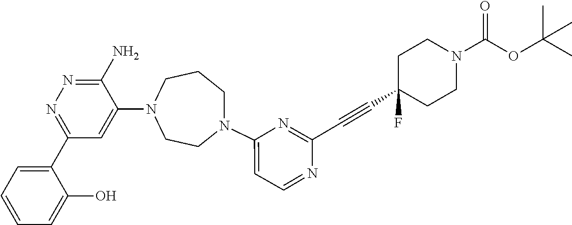

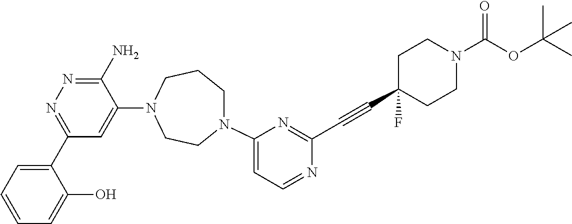

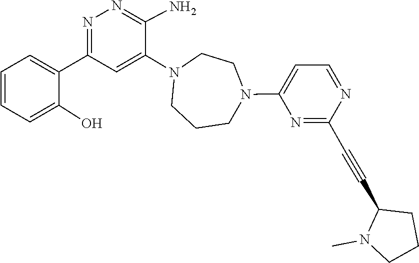 [SMILES: CN1CCC[C@@H]1C#Cc1nccc(N2CCCN(c3cc(-c4ccccc4O)nnc3N)CC2)n1]

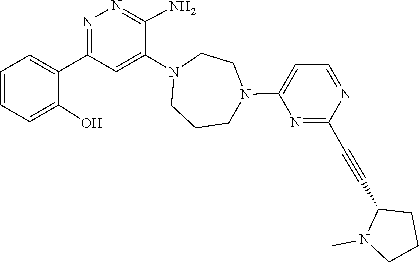 [SMILES: CN1CCC[C@H]1C#Cc1nccc(N2CCCN(c3cc(-c4ccccc4O)nnc3N)CC2)n1]

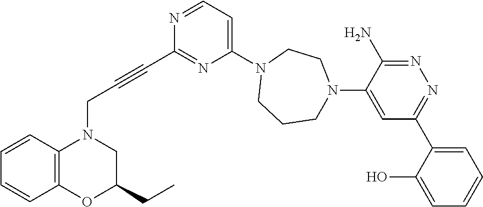 [SMILES: CC[C@@H]1CN(CC#Cc2nccc(N3CCCN(c4cc(-c5ccccc5O)nnc4N)CC3)n2)c2ccccc2O1]

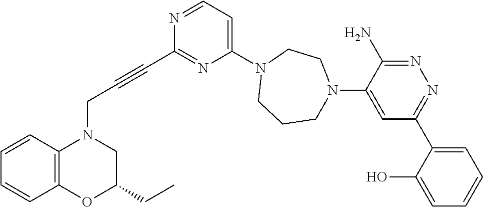 [SMILES: CC[C@H]1CN(CC#Cc2nccc(N3CCCN(c4cc(-c5ccccc5O)nnc4N)CC3)n2)c2ccccc2O1]

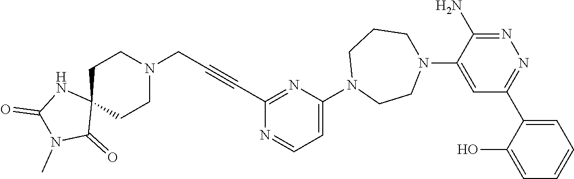

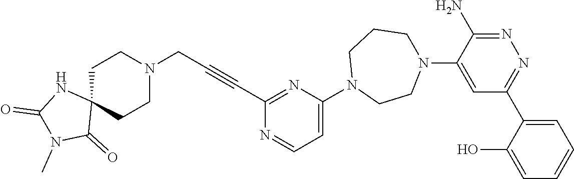

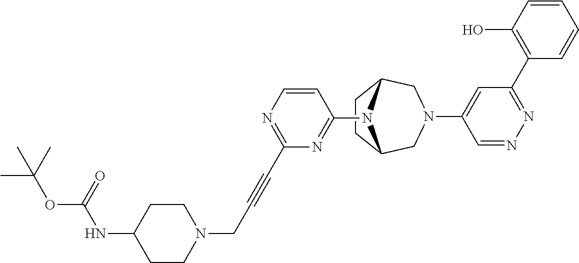 [SMILES: CC(C)(C)OC(=O)NC1CCN(CC#Cc2nccc(N3[C@@H]4CC[C@H]3CN(c3cnnc(-c5ccccc5O)c3)C4)n2)CC1]

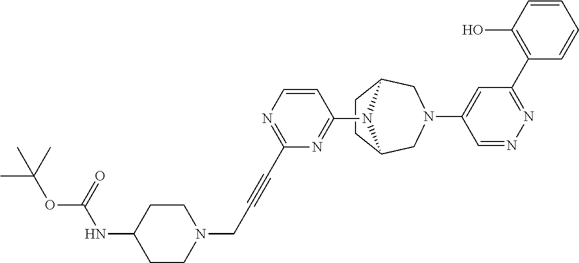 [SMILES: CC(C)(C)OC(=O)NC1CCN(CC#Cc2nccc(N3[C@@H]4CC[C@H]3CN(c3cnnc(-c5ccccc5O)c3)C4)n2)CC1]

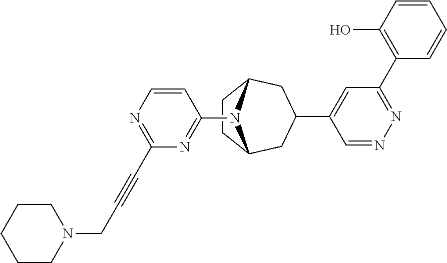 [SMILES: Oc1ccccc1-c1cc(C2C[C@H]3CC[C@@H](C2)N3c2ccnc(C#CCN3CCCCC3)n2)cnn1]

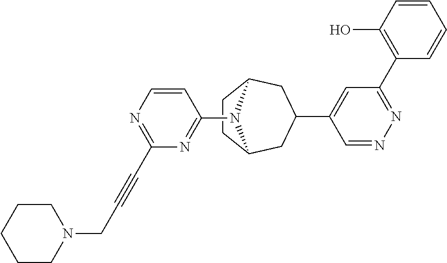 [SMILES: Oc1ccccc1-c1cc(C2C[C@H]3CC[C@@H](C2)N3c2ccnc(C#CCN3CCCCC3)n2)cnn1]

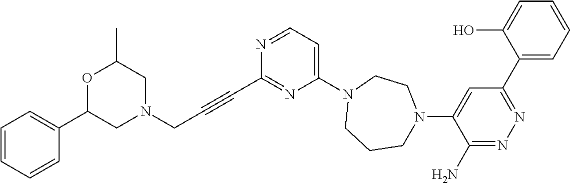 [SMILES: CC1CN(CC#Cc2nccc(N3CCCN(c4cc(-c5ccccc5O)nnc4N)CC3)n2)CC(c2ccccc2)O1]

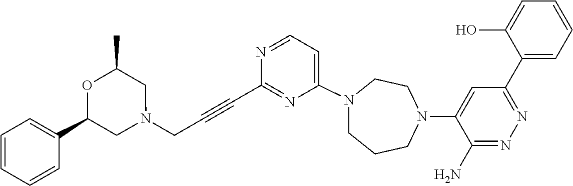 [SMILES: C[C@H]1CN(CC#Cc2nccc(N3CCCN(c4cc(-c5ccccc5O)nnc4N)CC3)n2)C[C@@H](c2ccccc2)O1]

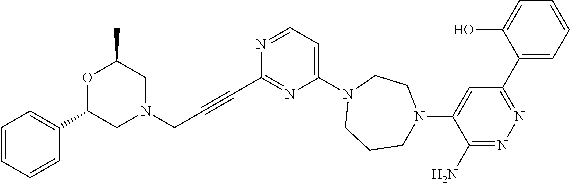 [SMILES: C[C@H]1CN(CC#Cc2nccc(N3CCCN(c4cc(-c5ccccc5O)nnc4N)CC3)n2)C[C@H](c2ccccc2)O1]

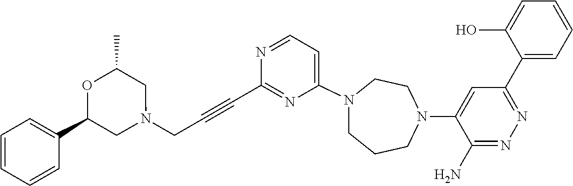 [SMILES: C[C@@H]1CN(CC#Cc2nccc(N3CCCN(c4cc(-c5ccccc5O)nnc4N)CC3)n2)C[C@@H](c2ccccc2)O1]

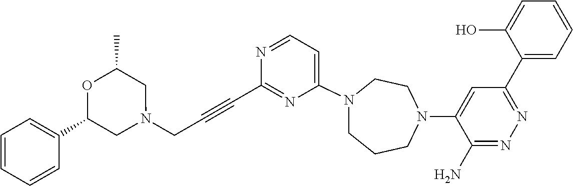 [SMILES: C[C@@H]1CN(CC#Cc2nccc(N3CCCN(c4cc(-c5ccccc5O)nnc4N)CC3)n2)C[C@H](c2ccccc2)O1]

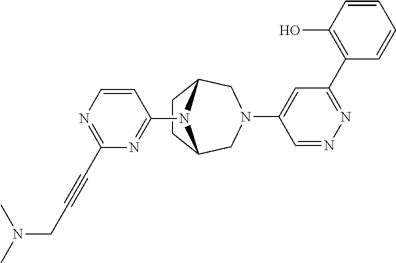 [SMILES: CN(C)CC#Cc1nccc(N2[C@@H]3CC[C@H]2CN(c2cnnc(-c4ccccc4O)c2)C3)n1]

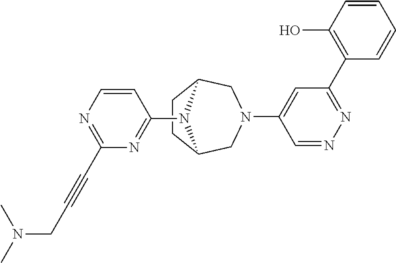 [SMILES: CN(C)CC#Cc1nccc(N2[C@@H]3CC[C@H]2CN(c2cnnc(-c4ccccc4O)c2)C3)n1]

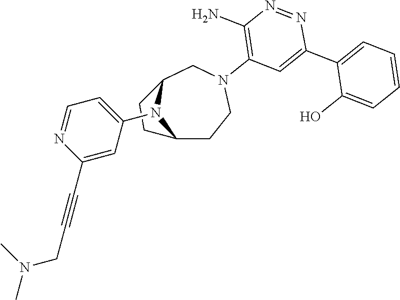 [SMILES: CN(C)CC#Cc1cc(N2[C@H]3CC[C@@H]2CN(c2cc(-c4ccccc4O)nnc2N)CC3)ccn1]

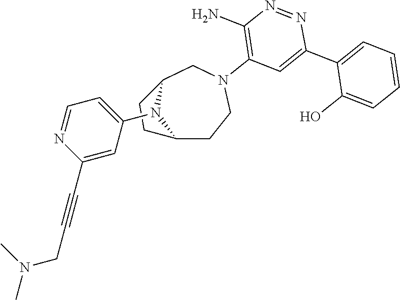 [SMILES: CN(C)CC#Cc1cc(N2[C@@H]3CC[C@H]2CN(c2cc(-c4ccccc4O)nnc2N)CC3)ccn1]

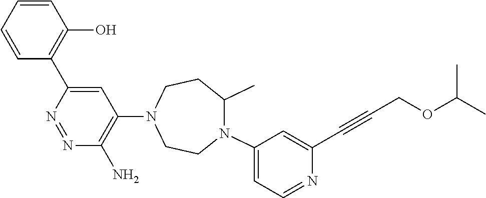 [SMILES: CC(C)OCC#Cc1cc(N2CCN(c3cc(-c4ccccc4O)nnc3N)CCC2C)ccn1]

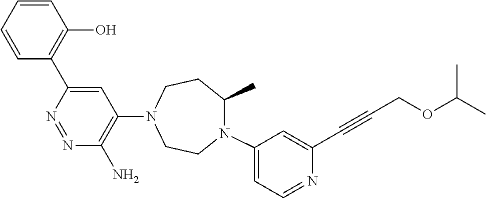 [SMILES: CC(C)OCC#Cc1cc(N2CCN(c3cc(-c4ccccc4O)nnc3N)CC[C@H]2C)ccn1]

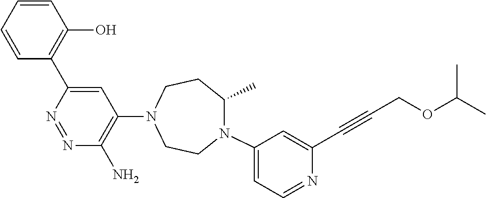 [SMILES: CC(C)OCC#Cc1cc(N2CCN(c3cc(-c4ccccc4O)nnc3N)CC[C@@H]2C)ccn1]

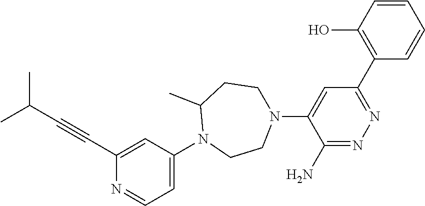 [SMILES: CC(C)C#Cc1cc(N2CCN(c3cc(-c4ccccc4O)nnc3N)CCC2C)ccn1]

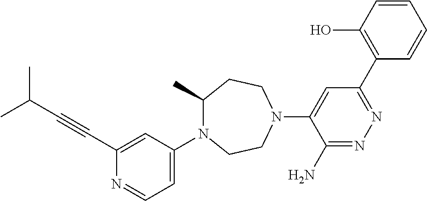 [SMILES: CC(C)C#Cc1cc(N2CCN(c3cc(-c4ccccc4O)nnc3N)CC[C@@H]2C)ccn1]

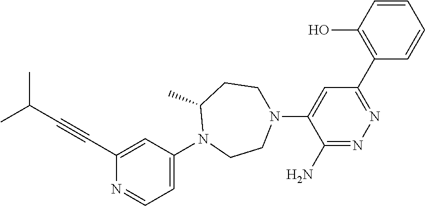 [SMILES: CC(C)C#Cc1cc(N2CCN(c3cc(-c4ccccc4O)nnc3N)CC[C@H]2C)ccn1]

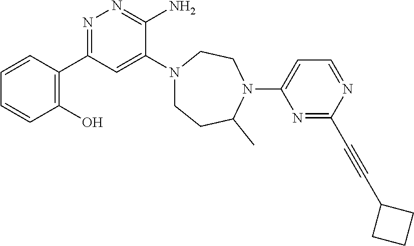 [SMILES: CC1CCN(c2cc(-c3ccccc3O)nnc2N)CCN1c1ccnc(C#CC2CCC2)n1]

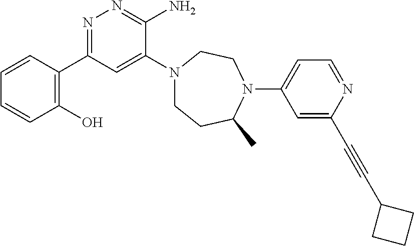 [SMILES: C[C@H]1CCN(c2cc(-c3ccccc3O)nnc2N)CCN1c1ccnc(C#CC2CCC2)c1]

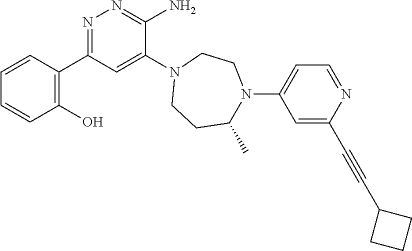 [SMILES: C[C@@H]1CCN(c2cc(-c3ccccc3O)nnc2N)CCN1c1ccnc(C#CC2CCC2)c1]

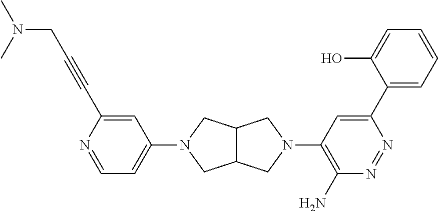 [SMILES: CN(C)CC#Cc1cc(N2CC3CN(c4cc(-c5ccccc5O)nnc4N)CC3C2)ccn1]

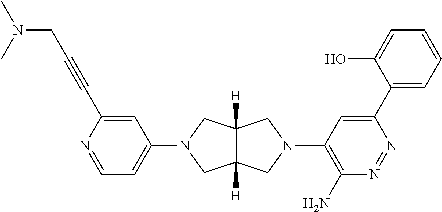 [SMILES: CN(C)CC#Cc1cc(N2C[C@@H]3CN(c4cc(-c5ccccc5O)nnc4N)C[C@@H]3C2)ccn1]

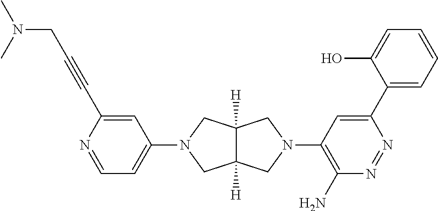 [SMILES: CN(C)CC#Cc1cc(N2C[C@@H]3CN(c4cc(-c5ccccc5O)nnc4N)C[C@@H]3C2)ccn1]

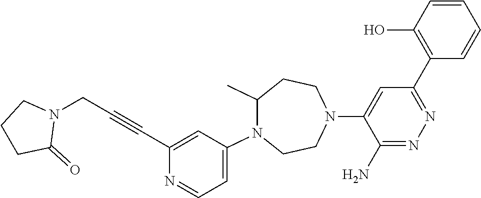 [SMILES: CC1CCN(c2cc(-c3ccccc3O)nnc2N)CCN1c1ccnc(C#CCN2CCCC2=O)c1]

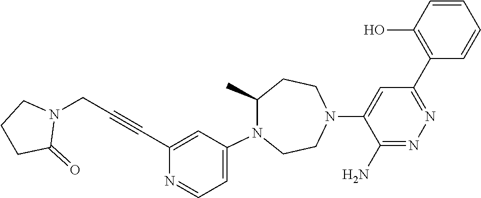 [SMILES: C[C@H]1CCN(c2cc(-c3ccccc3O)nnc2N)CCN1c1ccnc(C#CCN2CCCC2=O)c1]

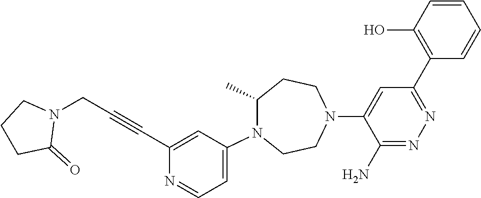 [SMILES: C[C@@H]1CCN(c2cc(-c3ccccc3O)nnc2N)CCN1c1ccnc(C#CCN2CCCC2=O)c1]

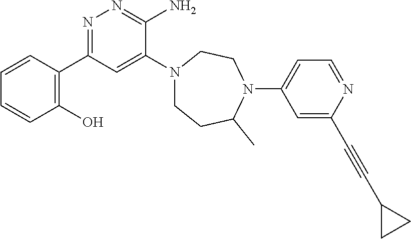 [SMILES: CC1CCN(c2cc(-c3ccccc3O)nnc2N)CCN1c1ccnc(C#CC2CC2)c1]

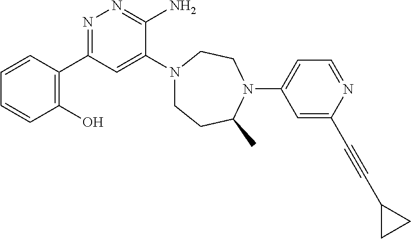 [SMILES: C[C@H]1CCN(c2cc(-c3ccccc3O)nnc2N)CCN1c1ccnc(C#CC2CC2)c1]

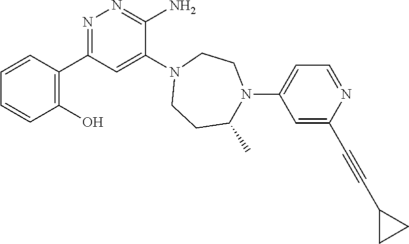 [SMILES: C[C@@H]1CCN(c2cc(-c3ccccc3O)nnc2N)CCN1c1ccnc(C#CC2CC2)c1]

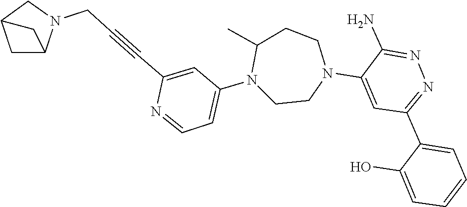

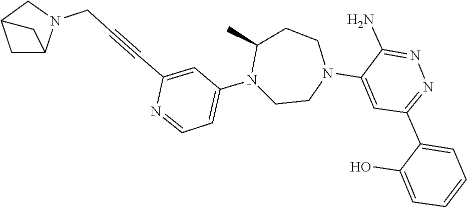

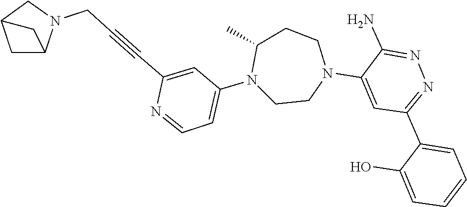

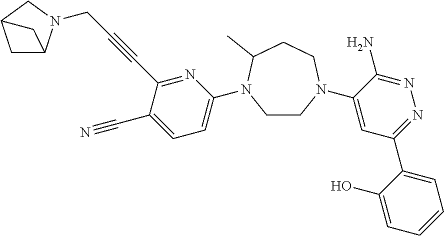 [SMILES: CC1CCN(c2cc(-c3ccccc3O)nnc2N)CCN1c1ccc(C#N)c(C#CCN2CC3CC2C3)n1]

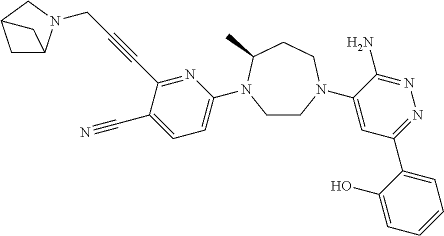 [SMILES: C[C@H]1CCN(c2cc(-c3ccccc3O)nnc2N)CCN1c1ccc(C#N)c(C#CCN2CC3CC2C3)n1]

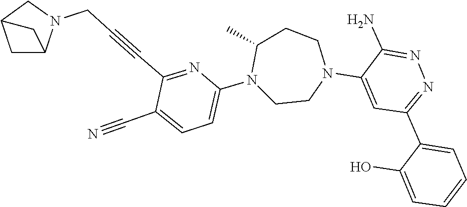

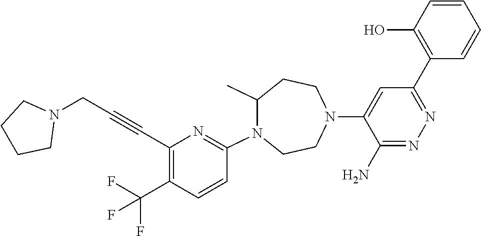 [SMILES: CC1CCN(c2cc(-c3ccccc3O)nnc2N)CCN1c1ccc(C(F)(F)F)c(C#CCN2CCCC2)n1]

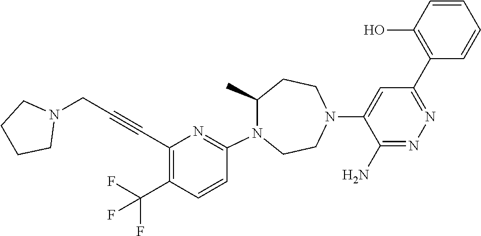 [SMILES: C[C@H]1CCN(c2cc(-c3ccccc3O)nnc2N)CCN1c1ccc(C(F)(F)F)c(C#CCN2CCCC2)n1]

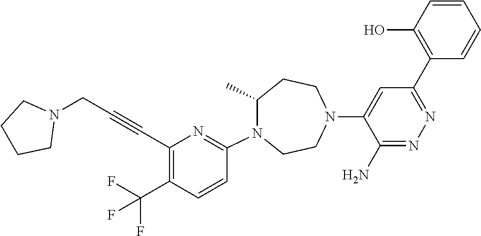 [SMILES: C[C@@H]1CCN(c2cc(-c3ccccc3O)nnc2N)CCN1c1ccc(C(F)(F)F)c(C#CCN2CCCC2)n1]

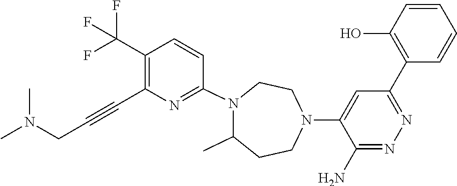 [SMILES: CC1CCN(c2cc(-c3ccccc3O)nnc2N)CCN1c1ccc(C(F)(F)F)c(C#CCN(C)C)n1]

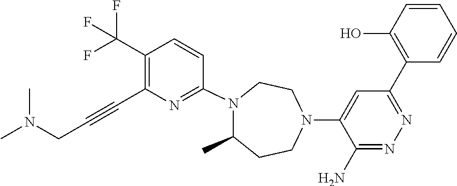 [SMILES: C[C@@H]1CCN(c2cc(-c3ccccc3O)nnc2N)CCN1c1ccc(C(F)(F)F)c(C#CCN(C)C)n1]

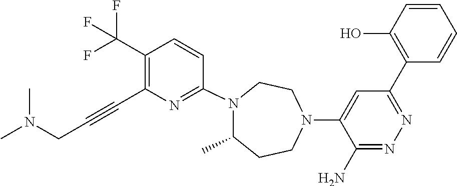 [SMILES: C[C@H]1CCN(c2cc(-c3ccccc3O)nnc2N)CCN1c1ccc(C(F)(F)F)c(C#CCN(C)C)n1]

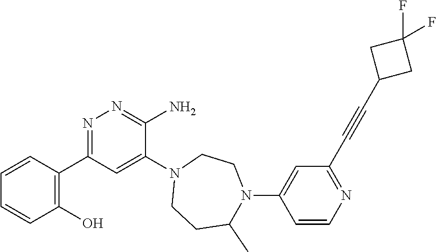 [SMILES: CC1CCN(c2cc(-c3ccccc3O)nnc2N)CCN1c1ccnc(C#CC2CC(F)(F)C2)c1]

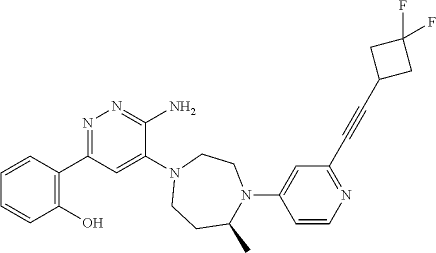 [SMILES: C[C@H]1CCN(c2cc(-c3ccccc3O)nnc2N)CCN1c1ccnc(C#CC2CC(F)(F)C2)c1]

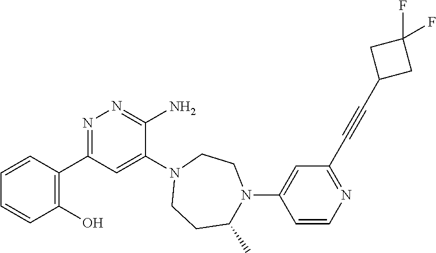 [SMILES: C[C@@H]1CCN(c2cc(-c3ccccc3O)nnc2N)CCN1c1ccnc(C#CC2CC(F)(F)C2)c1]

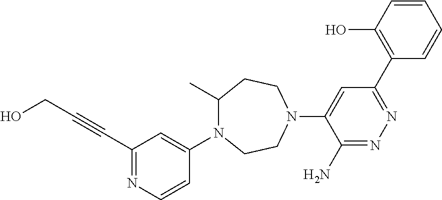 [SMILES: CC1CCN(c2cc(-c3ccccc3O)nnc2N)CCN1c1ccnc(C#CCO)c1]

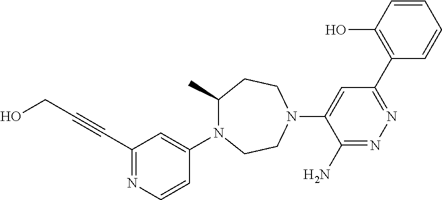 [SMILES: C[C@H]1CCN(c2cc(-c3ccccc3O)nnc2N)CCN1c1ccnc(C#CCO)c1]

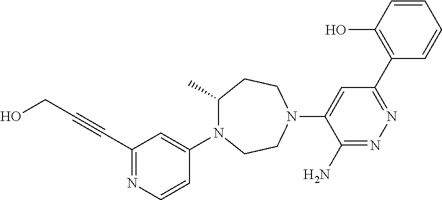 [SMILES: C[C@@H]1CCN(c2cc(-c3ccccc3O)nnc2N)CCN1c1ccnc(C#CCO)c1]

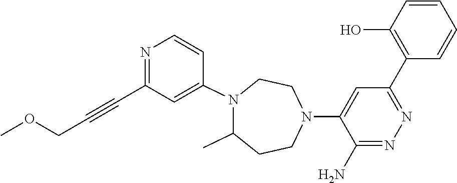 [SMILES: COCC#Cc1cc(N2CCN(c3cc(-c4ccccc4O)nnc3N)CCC2C)ccn1]

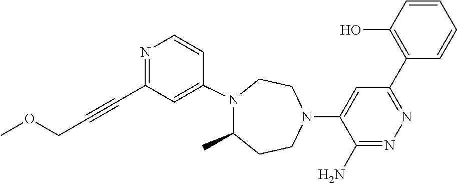 [SMILES: COCC#Cc1cc(N2CCN(c3cc(-c4ccccc4O)nnc3N)CC[C@H]2C)ccn1]

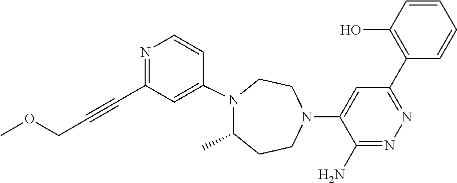 [SMILES: COCC#Cc1cc(N2CCN(c3cc(-c4ccccc4O)nnc3N)CC[C@@H]2C)ccn1]

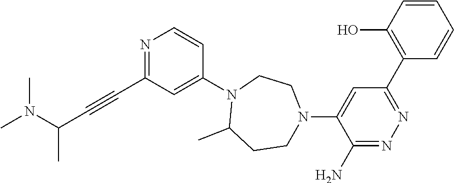 [SMILES: CC(C#Cc1cc(N2CCN(c3cc(-c4ccccc4O)nnc3N)CCC2C)ccn1)N(C)C]

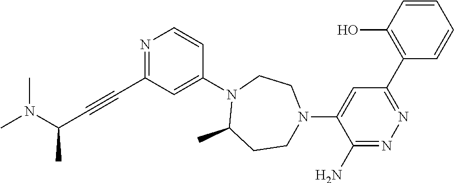 [SMILES: C[C@H](C#Cc1cc(N2CCN(c3cc(-c4ccccc4O)nnc3N)CC[C@H]2C)ccn1)N(C)C]

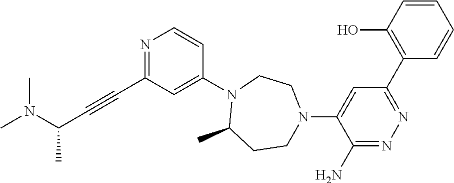 [SMILES: C[C@@H]1CCN(c2cc(-c3ccccc3O)nnc2N)CCN1c1ccnc(C#C[C@H](C)N(C)C)c1]

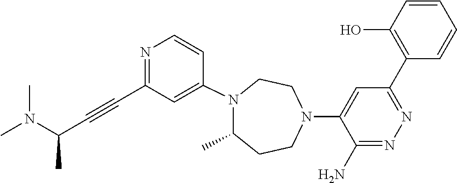 [SMILES: C[C@H](C#Cc1cc(N2CCN(c3cc(-c4ccccc4O)nnc3N)CC[C@@H]2C)ccn1)N(C)C]

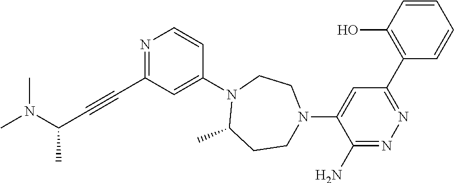 [SMILES: C[C@@H](C#Cc1cc(N2CCN(c3cc(-c4ccccc4O)nnc3N)CC[C@@H]2C)ccn1)N(C)C]

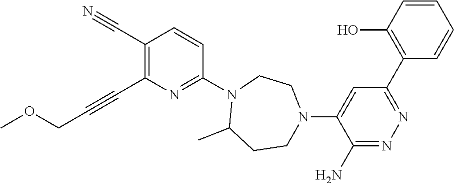 [SMILES: COCC#Cc1nc(N2CCN(c3cc(-c4ccccc4O)nnc3N)CCC2C)ccc1C#N]

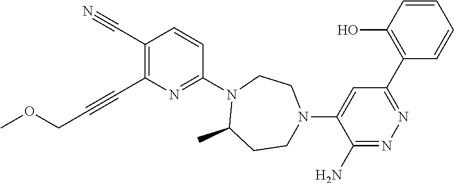 [SMILES: COCC#Cc1nc(N2CCN(c3cc(-c4ccccc4O)nnc3N)CC[C@H]2C)ccc1C#N]

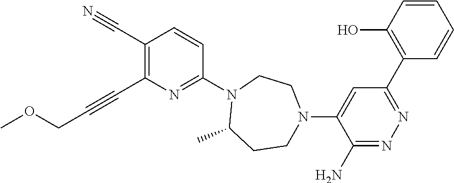 [SMILES: COCC#Cc1nc(N2CCN(c3cc(-c4ccccc4O)nnc3N)CC[C@@H]2C)ccc1C#N]

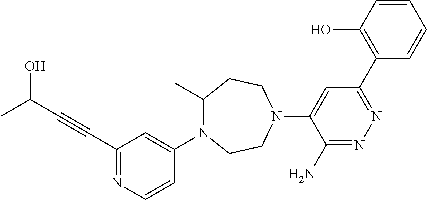 [SMILES: CC(O)C#Cc1cc(N2CCN(c3cc(-c4ccccc4O)nnc3N)CCC2C)ccn1]

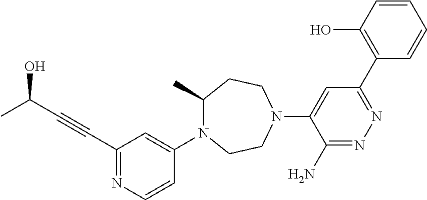 [SMILES: C[C@@H](O)C#Cc1cc(N2CCN(c3cc(-c4ccccc4O)nnc3N)CC[C@@H]2C)ccn1]

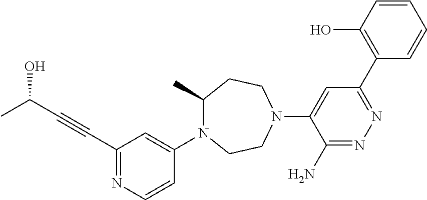 [SMILES: C[C@H](O)C#Cc1cc(N2CCN(c3cc(-c4ccccc4O)nnc3N)CC[C@@H]2C)ccn1]

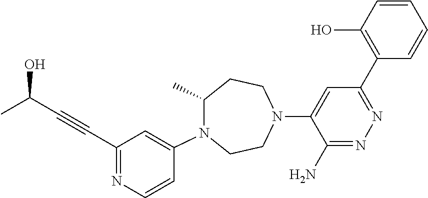 [SMILES: C[C@@H]1CCN(c2cc(-c3ccccc3O)nnc2N)CCN1c1ccnc(C#C[C@@H](C)O)c1]

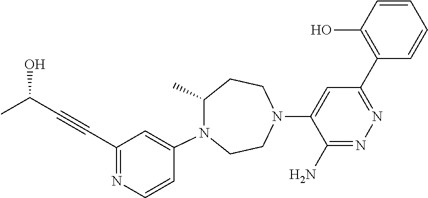 [SMILES: C[C@H](O)C#Cc1cc(N2CCN(c3cc(-c4ccccc4O)nnc3N)CC[C@H]2C)ccn1]

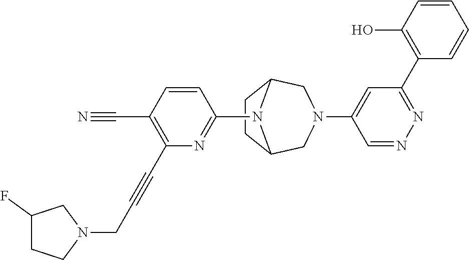 [SMILES: N#Cc1ccc(N2C3CCC2CN(c2cnnc(-c4ccccc4O)c2)C3)nc1C#CCN1CCC(F)C1]

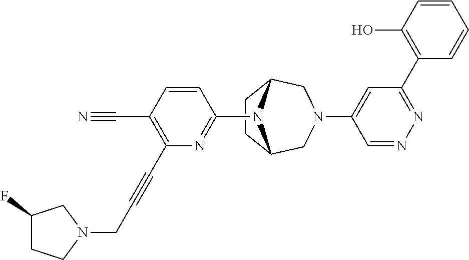 [SMILES: N#Cc1ccc(N2[C@@H]3CC[C@H]2CN(c2cnnc(-c4ccccc4O)c2)C3)nc1C#CCN1CC[C@@H](F)C1]

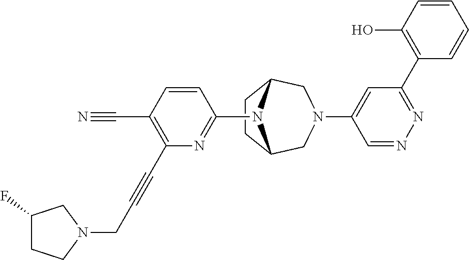 [SMILES: N#Cc1ccc(N2[C@@H]3CC[C@H]2CN(c2cnnc(-c4ccccc4O)c2)C3)nc1C#CCN1CC[C@H](F)C1]

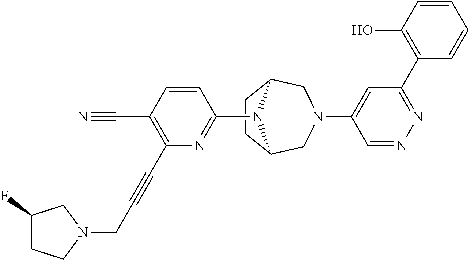 [SMILES: N#Cc1ccc(N2[C@@H]3CC[C@H]2CN(c2cnnc(-c4ccccc4O)c2)C3)nc1C#CCN1CC[C@@H](F)C1]

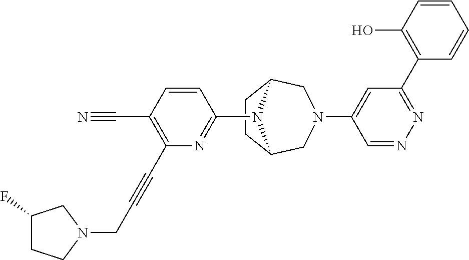 [SMILES: N#Cc1ccc(N2[C@@H]3CC[C@H]2CN(c2cnnc(-c4ccccc4O)c2)C3)nc1C#CCN1CC[C@H](F)C1]

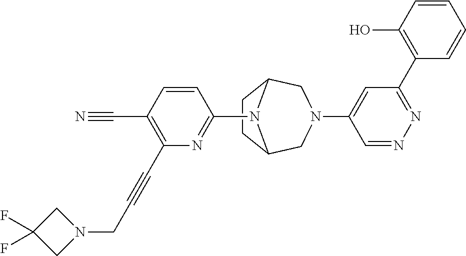 [SMILES: N#Cc1ccc(N2C3CCC2CN(c2cnnc(-c4ccccc4O)c2)C3)nc1C#CCN1CC(F)(F)C1]

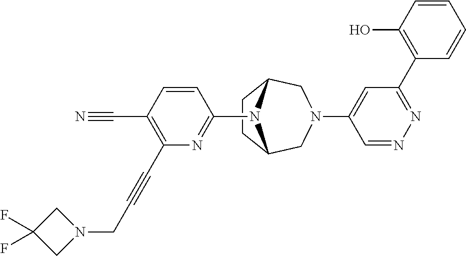 [SMILES: N#Cc1ccc(N2[C@@H]3CC[C@H]2CN(c2cnnc(-c4ccccc4O)c2)C3)nc1C#CCN1CC(F)(F)C1]

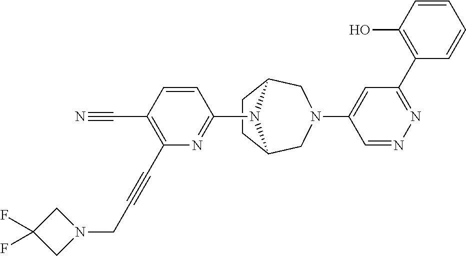 [SMILES: N#Cc1ccc(N2[C@@H]3CC[C@H]2CN(c2cnnc(-c4ccccc4O)c2)C3)nc1C#CCN1CC(F)(F)C1]

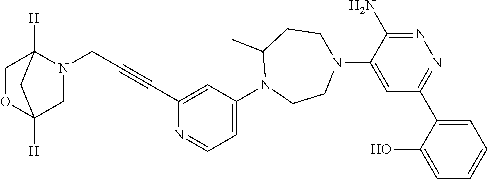 [SMILES: CC1CCN(c2cc(-c3ccccc3O)nnc2N)CCN1c1ccnc(C#CCN2CC3CC2CO3)c1]

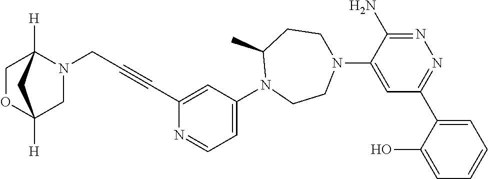 [SMILES: C[C@H]1CCN(c2cc(-c3ccccc3O)nnc2N)CCN1c1ccnc(C#CCN2C[C@H]3C[C@@H]2CO3)c1]

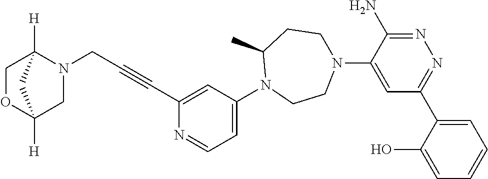 [SMILES: C[C@H]1CCN(c2cc(-c3ccccc3O)nnc2N)CCN1c1ccnc(C#CCN2C[C@@H]3C[C@H]2CO3)c1]

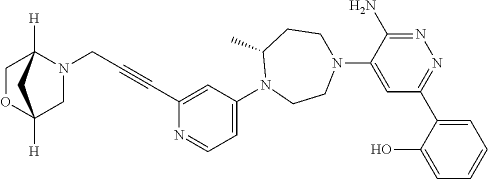 [SMILES: C[C@@H]1CCN(c2cc(-c3ccccc3O)nnc2N)CCN1c1ccnc(C#CCN2C[C@H]3C[C@@H]2CO3)c1]

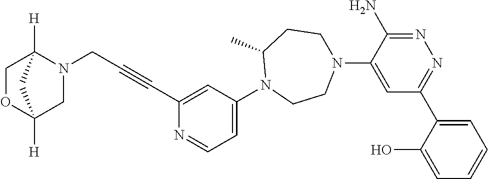 [SMILES: C[C@@H]1CCN(c2cc(-c3ccccc3O)nnc2N)CCN1c1ccnc(C#CCN2C[C@@H]3C[C@H]2CO3)c1]

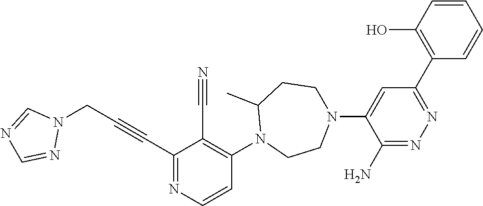 [SMILES: CC1CCN(c2cc(-c3ccccc3O)nnc2N)CCN1c1ccnc(C#CCn2cncn2)c1C#N]

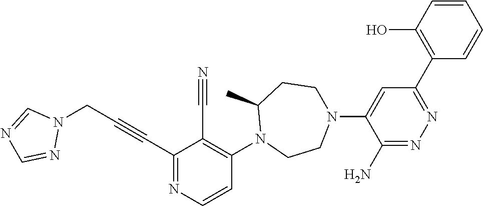 [SMILES: C[C@H]1CCN(c2cc(-c3ccccc3O)nnc2N)CCN1c1ccnc(C#CCn2cncn2)c1C#N]

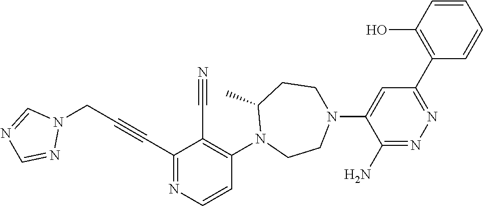 [SMILES: C[C@@H]1CCN(c2cc(-c3ccccc3O)nnc2N)CCN1c1ccnc(C#CCn2cncn2)c1C#N]

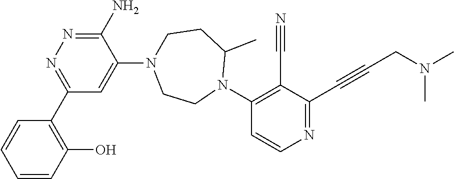 [SMILES: CC1CCN(c2cc(-c3ccccc3O)nnc2N)CCN1c1ccnc(C#CCN(C)C)c1C#N]

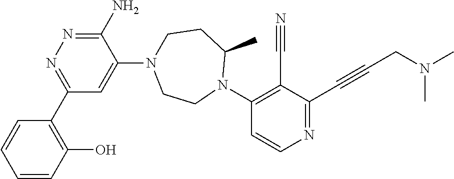 [SMILES: C[C@@H]1CCN(c2cc(-c3ccccc3O)nnc2N)CCN1c1ccnc(C#CCN(C)C)c1C#N]

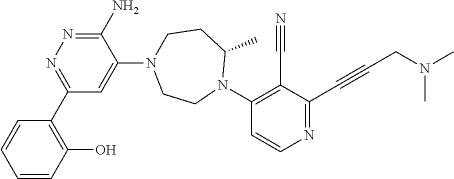 [SMILES: C[C@H]1CCN(c2cc(-c3ccccc3O)nnc2N)CCN1c1ccnc(C#CCN(C)C)c1C#N]

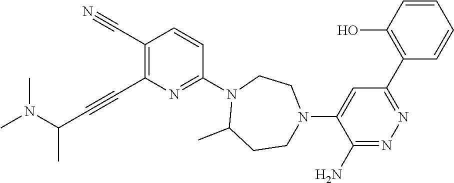 [SMILES: CC(C#Cc1nc(N2CCN(c3cc(-c4ccccc4O)nnc3N)CCC2C)ccc1C#N)N(C)C]

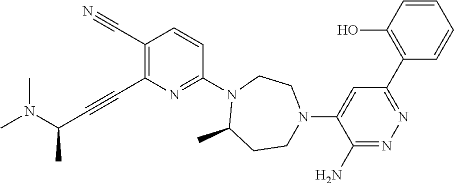 [SMILES: C[C@H](C#Cc1nc(N2CCN(c3cc(-c4ccccc4O)nnc3N)CC[C@H]2C)ccc1C#N)N(C)C]

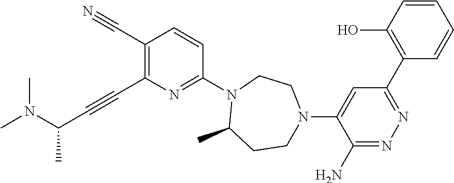 [SMILES: C[C@@H]1CCN(c2cc(-c3ccccc3O)nnc2N)CCN1c1ccc(C#N)c(C#C[C@H](C)N(C)C)n1]

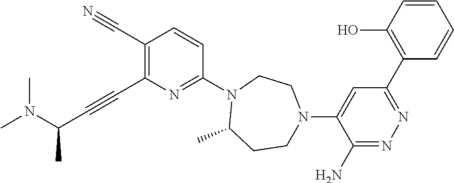 [SMILES: C[C@H](C#Cc1nc(N2CCN(c3cc(-c4ccccc4O)nnc3N)CC[C@@H]2C)ccc1C#N)N(C)C]

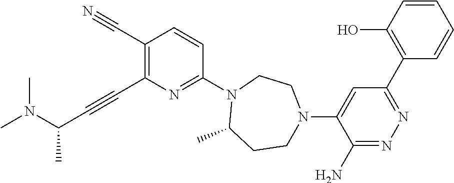 [SMILES: C[C@@H](C#Cc1nc(N2CCN(c3cc(-c4ccccc4O)nnc3N)CC[C@@H]2C)ccc1C#N)N(C)C]

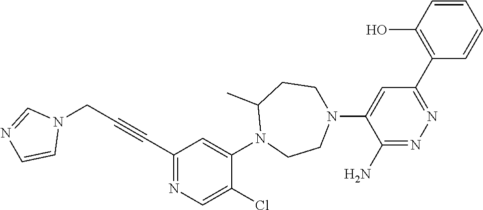 [SMILES: CC1CCN(c2cc(-c3ccccc3O)nnc2N)CCN1c1cc(C#CCn2ccnc2)ncc1Cl]

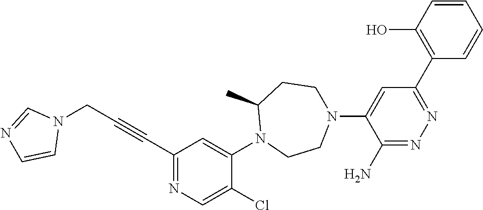 [SMILES: C[C@H]1CCN(c2cc(-c3ccccc3O)nnc2N)CCN1c1cc(C#CCn2ccnc2)ncc1Cl]

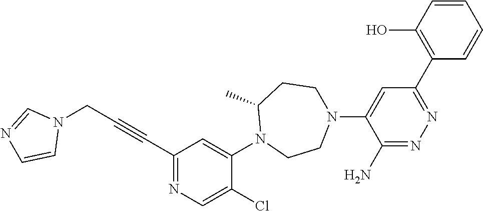 [SMILES: C[C@@H]1CCN(c2cc(-c3ccccc3O)nnc2N)CCN1c1cc(C#CCn2ccnc2)ncc1Cl]

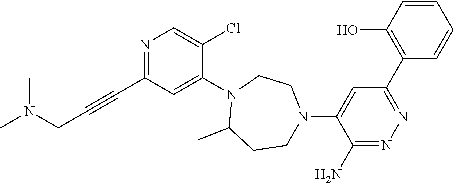 [SMILES: CC1CCN(c2cc(-c3ccccc3O)nnc2N)CCN1c1cc(C#CCN(C)C)ncc1Cl]

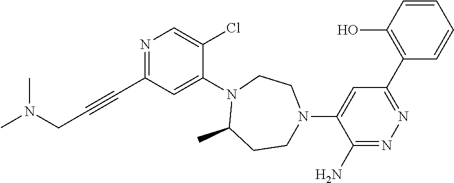 [SMILES: C[C@@H]1CCN(c2cc(-c3ccccc3O)nnc2N)CCN1c1cc(C#CCN(C)C)ncc1Cl]

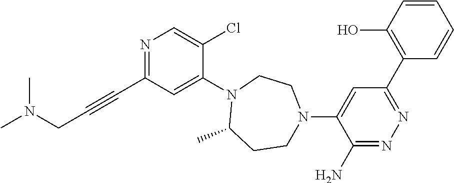 [SMILES: C[C@H]1CCN(c2cc(-c3ccccc3O)nnc2N)CCN1c1cc(C#CCN(C)C)ncc1Cl]

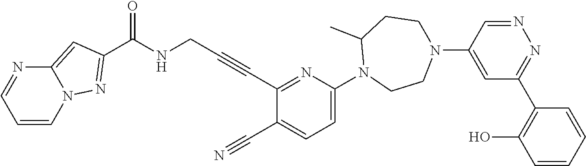 [SMILES: CC1CCN(c2cnnc(-c3ccccc3O)c2)CCN1c1ccc(C#N)c(C#CCNC(=O)c2cc3ncccn3n2)n1]

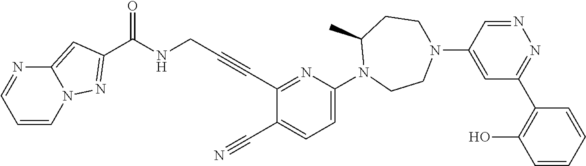 [SMILES: C[C@H]1CCN(c2cnnc(-c3ccccc3O)c2)CCN1c1ccc(C#N)c(C#CCNC(=O)c2cc3ncccn3n2)n1]

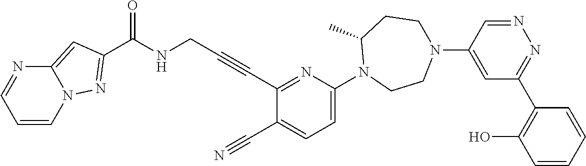 [SMILES: C[C@@H]1CCN(c2cnnc(-c3ccccc3O)c2)CCN1c1ccc(C#N)c(C#CCNC(=O)c2cc3ncccn3n2)n1]

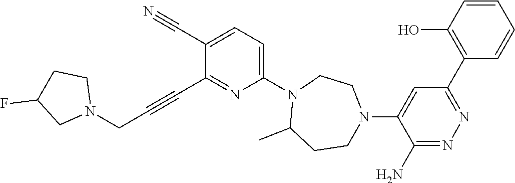 [SMILES: CC1CCN(c2cc(-c3ccccc3O)nnc2N)CCN1c1ccc(C#N)c(C#CCN2CCC(F)C2)n1]

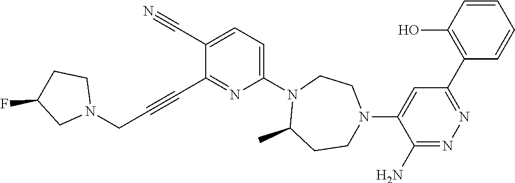 [SMILES: C[C@@H]1CCN(c2cc(-c3ccccc3O)nnc2N)CCN1c1ccc(C#N)c(C#CCN2CC[C@H](F)C2)n1]

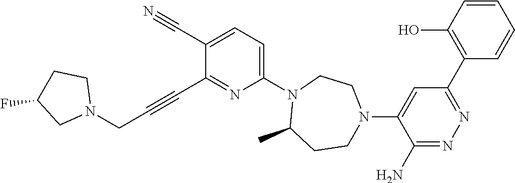 [SMILES: C[C@@H]1CCN(c2cc(-c3ccccc3O)nnc2N)CCN1c1ccc(C#N)c(C#CCN2CC[C@@H](F)C2)n1]

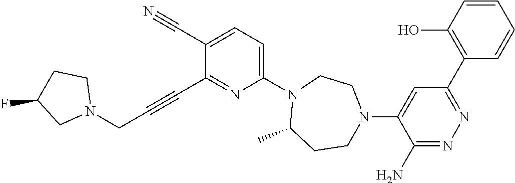 [SMILES: C[C@H]1CCN(c2cc(-c3ccccc3O)nnc2N)CCN1c1ccc(C#N)c(C#CCN2CC[C@H](F)C2)n1]

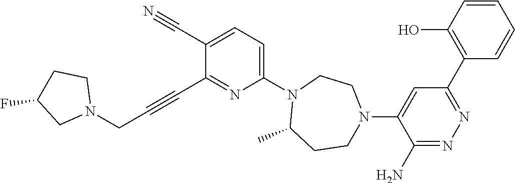 [SMILES: C[C@H]1CCN(c2cc(-c3ccccc3O)nnc2N)CCN1c1ccc(C#N)c(C#CCN2CC[C@@H](F)C2)n1]

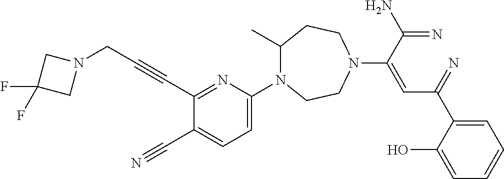

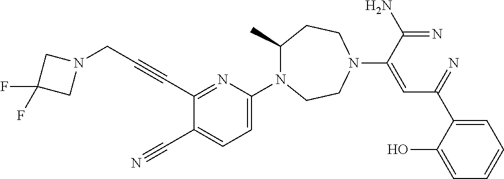

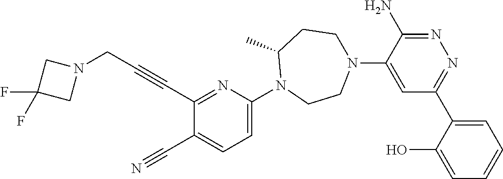 [SMILES: C[C@@H]1CCN(c2cc(-c3ccccc3O)nnc2N)CCN1c1ccc(C#N)c(C#CCN2CC(F)(F)C2)n1]

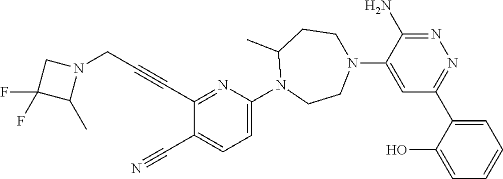 [SMILES: CC1CCN(c2cc(-c3ccccc3O)nnc2N)CCN1c1ccc(C#N)c(C#CCN2CC(F)(F)C2C)n1]

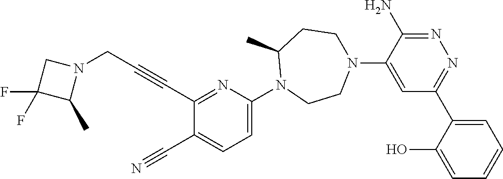 [SMILES: C[C@@H]1N(CC#Cc2nc(N3CCN(c4cc(-c5ccccc5O)nnc4N)CC[C@@H]3C)ccc2C#N)CC1(F)F]

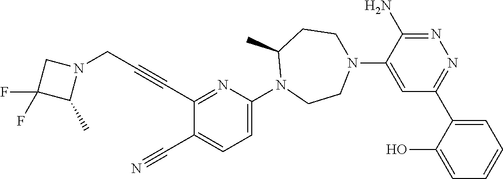 [SMILES: C[C@H]1CCN(c2cc(-c3ccccc3O)nnc2N)CCN1c1ccc(C#N)c(C#CCN2CC(F)(F)[C@H]2C)n1]

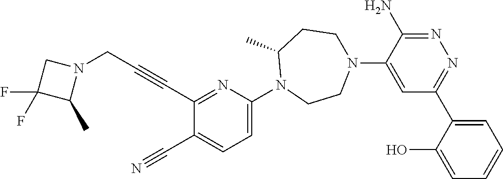 [SMILES: C[C@@H]1CCN(c2cc(-c3ccccc3O)nnc2N)CCN1c1ccc(C#N)c(C#CCN2CC(F)(F)[C@@H]2C)n1]

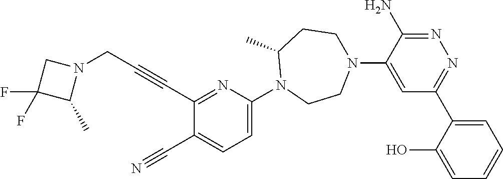 [SMILES: C[C@@H]1CCN(c2cc(-c3ccccc3O)nnc2N)CCN1c1ccc(C#N)c(C#CCN2CC(F)(F)[C@H]2C)n1]

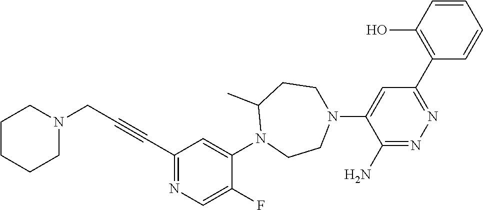 [SMILES: CC1CCN(c2cc(-c3ccccc3O)nnc2N)CCN1c1cc(C#CCN2CCCCC2)ncc1F]

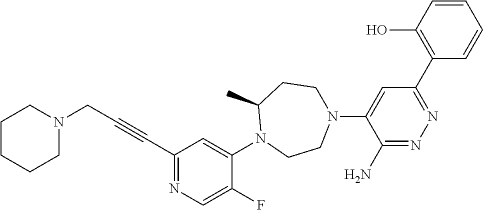 [SMILES: C[C@H]1CCN(c2cc(-c3ccccc3O)nnc2N)CCN1c1cc(C#CCN2CCCCC2)ncc1F]

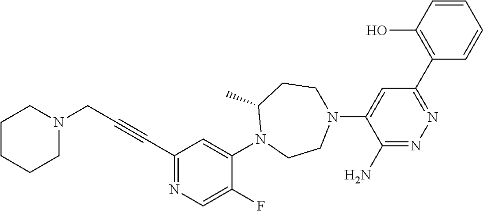 [SMILES: C[C@@H]1CCN(c2cc(-c3ccccc3O)nnc2N)CCN1c1cc(C#CCN2CCCCC2)ncc1F]

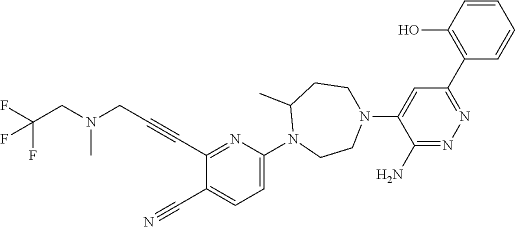 [SMILES: CC1CCN(c2cc(-c3ccccc3O)nnc2N)CCN1c1ccc(C#N)c(C#CCN(C)CC(F)(F)F)n1]

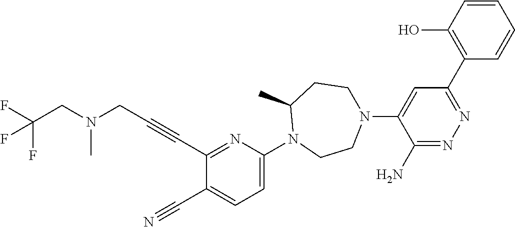 [SMILES: C[C@H]1CCN(c2cc(-c3ccccc3O)nnc2N)CCN1c1ccc(C#N)c(C#CCN(C)CC(F)(F)F)n1]

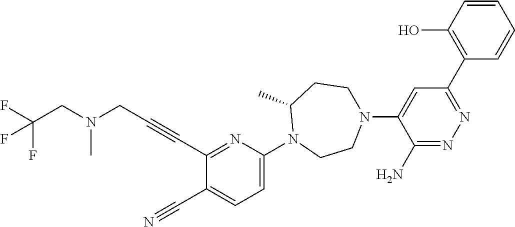 [SMILES: C[C@@H]1CCN(c2cc(-c3ccccc3O)nnc2N)CCN1c1ccc(C#N)c(C#CCN(C)CC(F)(F)F)n1]

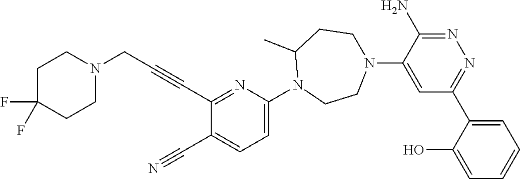 [SMILES: CC1CCN(c2cc(-c3ccccc3O)nnc2N)CCN1c1ccc(C#N)c(C#CCN2CCC(F)(F)CC2)n1]

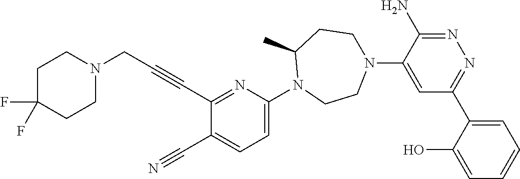 [SMILES: C[C@H]1CCN(c2cc(-c3ccccc3O)nnc2N)CCN1c1ccc(C#N)c(C#CCN2CCC(F)(F)CC2)n1]

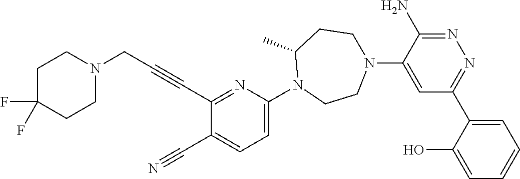 [SMILES: C[C@@H]1CCN(c2cc(-c3ccccc3O)nnc2N)CCN1c1ccc(C#N)c(C#CCN2CCC(F)(F)CC2)n1]

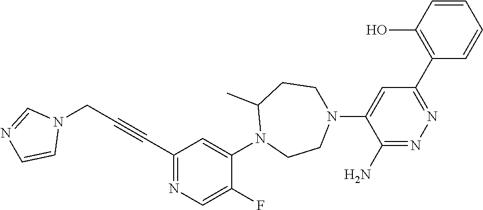 [SMILES: CC1CCN(c2cc(-c3ccccc3O)nnc2N)CCN1c1cc(C#CCn2ccnc2)ncc1F]

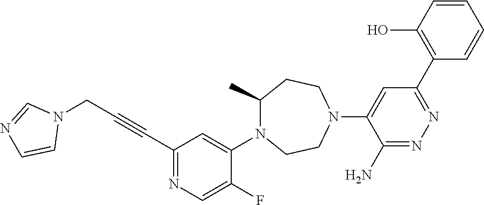 [SMILES: C[C@H]1CCN(c2cc(-c3ccccc3O)nnc2N)CCN1c1cc(C#CCn2ccnc2)ncc1F]

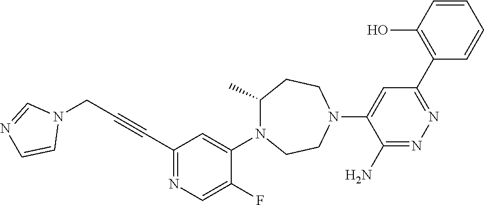 [SMILES: C[C@@H]1CCN(c2cc(-c3ccccc3O)nnc2N)CCN1c1cc(C#CCn2ccnc2)ncc1F]

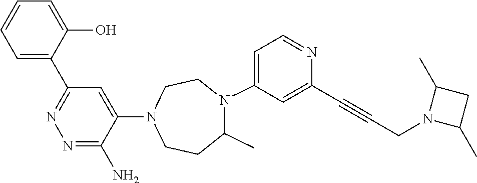 [SMILES: CC1CCN(c2cc(-c3ccccc3O)nnc2N)CCN1c1ccnc(C#CCN2C(C)CC2C)c1]

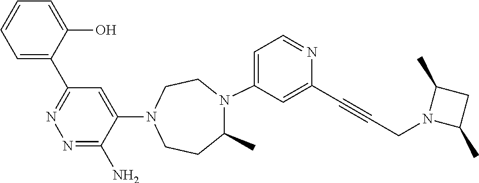 [SMILES: C[C@@H]1C[C@H](C)N1CC#Cc1cc(N2CCN(c3cc(-c4ccccc4O)nnc3N)CC[C@@H]2C)ccn1]

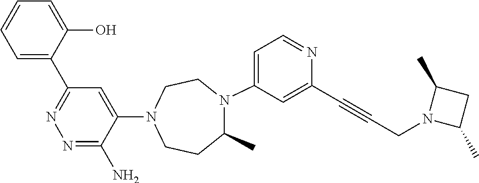 [SMILES: C[C@H]1CCN(c2cc(-c3ccccc3O)nnc2N)CCN1c1ccnc(C#CCN2[C@@H](C)C[C@@H]2C)c1]

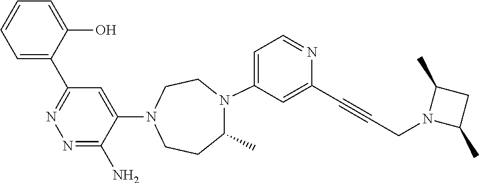 [SMILES: C[C@@H]1CCN(c2cc(-c3ccccc3O)nnc2N)CCN1c1ccnc(C#CCN2[C@H](C)C[C@@H]2C)c1]

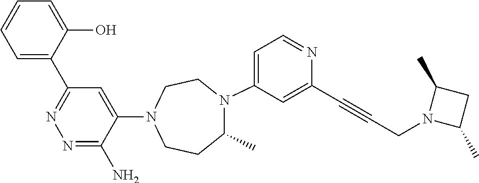 [SMILES: C[C@@H]1CCN(c2cc(-c3ccccc3O)nnc2N)CCN1c1ccnc(C#CCN2[C@@H](C)C[C@@H]2C)c1]

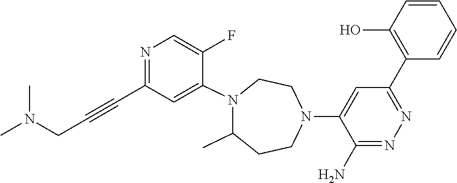 [SMILES: CC1CCN(c2cc(-c3ccccc3O)nnc2N)CCN1c1cc(C#CCN(C)C)ncc1F]

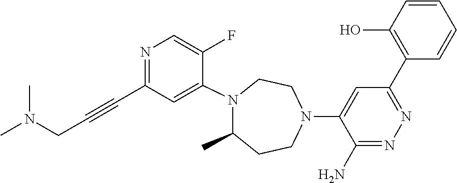 [SMILES: C[C@@H]1CCN(c2cc(-c3ccccc3O)nnc2N)CCN1c1cc(C#CCN(C)C)ncc1F]

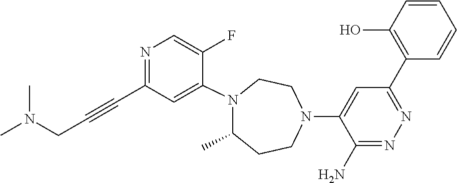 [SMILES: C[C@H]1CCN(c2cc(-c3ccccc3O)nnc2N)CCN1c1cc(C#CCN(C)C)ncc1F]

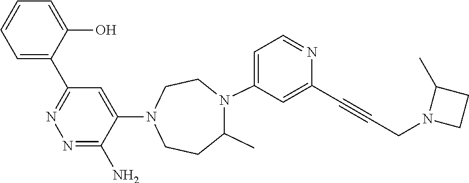 [SMILES: CC1CCN1CC#Cc1cc(N2CCN(c3cc(-c4ccccc4O)nnc3N)CCC2C)ccn1]

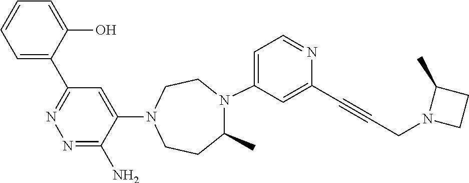 [SMILES: C[C@H]1CCN1CC#Cc1cc(N2CCN(c3cc(-c4ccccc4O)nnc3N)CC[C@@H]2C)ccn1]

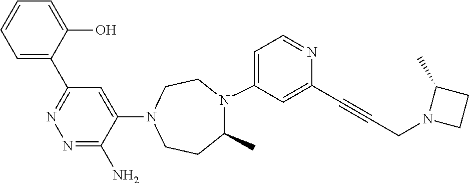 [SMILES: C[C@@H]1CCN1CC#Cc1cc(N2CCN(c3cc(-c4ccccc4O)nnc3N)CC[C@@H]2C)ccn1]

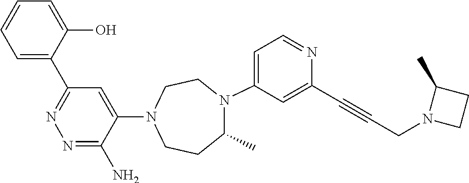 [SMILES: C[C@@H]1CCN(c2cc(-c3ccccc3O)nnc2N)CCN1c1ccnc(C#CCN2CC[C@@H]2C)c1]

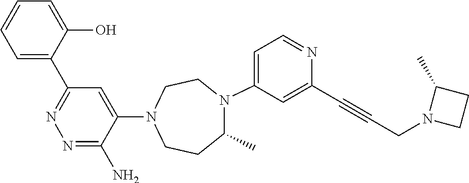 [SMILES: C[C@@H]1CCN1CC#Cc1cc(N2CCN(c3cc(-c4ccccc4O)nnc3N)CC[C@H]2C)ccn1]

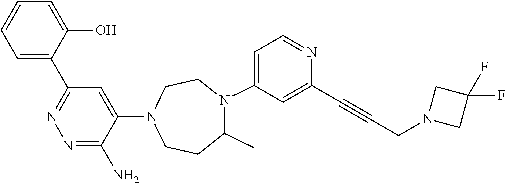 [SMILES: CC1CCN(c2cc(-c3ccccc3O)nnc2N)CCN1c1ccnc(C#CCN2CC(F)(F)C2)c1]

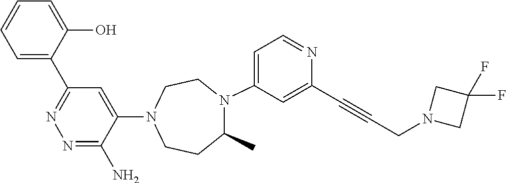 [SMILES: C[C@H]1CCN(c2cc(-c3ccccc3O)nnc2N)CCN1c1ccnc(C#CCN2CC(F)(F)C2)c1]

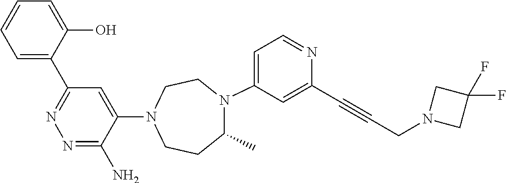 [SMILES: C[C@@H]1CCN(c2cc(-c3ccccc3O)nnc2N)CCN1c1ccnc(C#CCN2CC(F)(F)C2)c1]

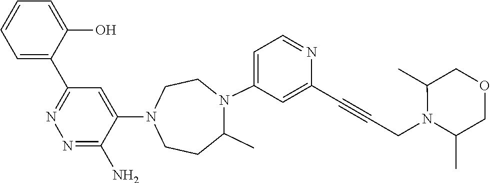 [SMILES: CC1CCN(c2cc(-c3ccccc3O)nnc2N)CCN1c1ccnc(C#CCN2C(C)COCC2C)c1]

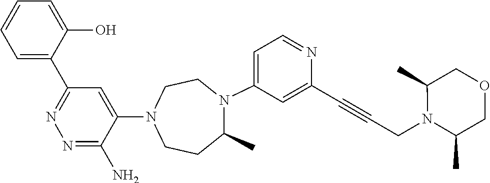 [SMILES: C[C@@H]1COC[C@H](C)N1CC#Cc1cc(N2CCN(c3cc(-c4ccccc4O)nnc3N)CC[C@@H]2C)ccn1]

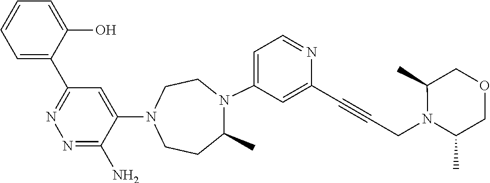 [SMILES: C[C@H]1CCN(c2cc(-c3ccccc3O)nnc2N)CCN1c1ccnc(C#CCN2[C@@H](C)COC[C@@H]2C)c1]

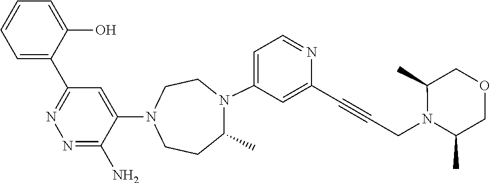 [SMILES: C[C@@H]1CCN(c2cc(-c3ccccc3O)nnc2N)CCN1c1ccnc(C#CCN2[C@H](C)COC[C@@H]2C)c1]

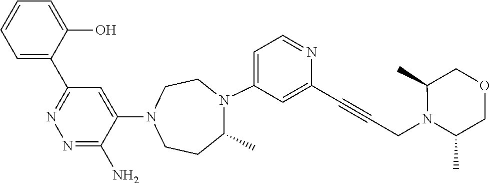 [SMILES: C[C@@H]1CCN(c2cc(-c3ccccc3O)nnc2N)CCN1c1ccnc(C#CCN2[C@@H](C)COC[C@@H]2C)c1]

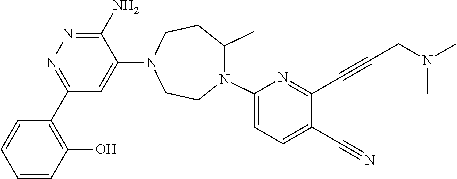 [SMILES: CC1CCN(c2cc(-c3ccccc3O)nnc2N)CCN1c1ccc(C#N)c(C#CCN(C)C)n1]

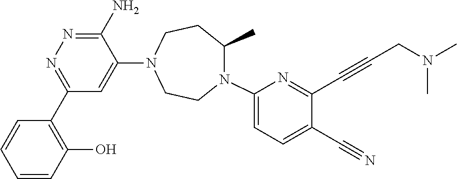 [SMILES: C[C@@H]1CCN(c2cc(-c3ccccc3O)nnc2N)CCN1c1ccc(C#N)c(C#CCN(C)C)n1]

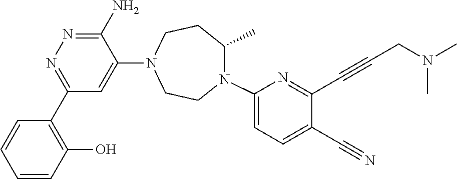 [SMILES: C[C@H]1CCN(c2cc(-c3ccccc3O)nnc2N)CCN1c1ccc(C#N)c(C#CCN(C)C)n1]

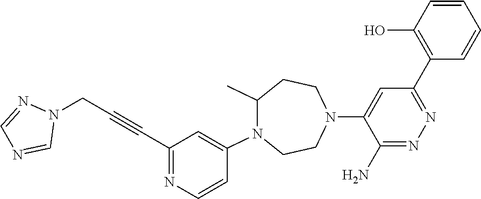 [SMILES: CC1CCN(c2cc(-c3ccccc3O)nnc2N)CCN1c1ccnc(C#CCn2cncn2)c1]

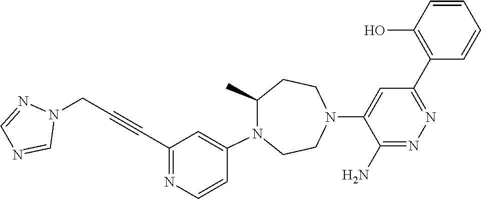 [SMILES: C[C@H]1CCN(c2cc(-c3ccccc3O)nnc2N)CCN1c1ccnc(C#CCn2cncn2)c1]

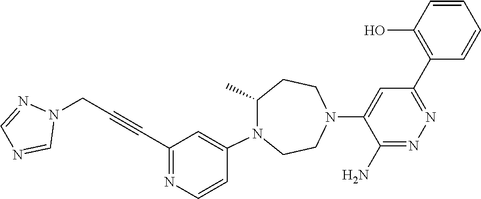 [SMILES: C[C@@H]1CCN(c2cc(-c3ccccc3O)nnc2N)CCN1c1ccnc(C#CCn2cncn2)c1]

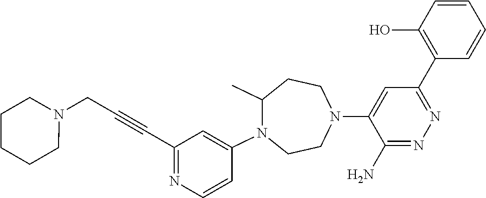 [SMILES: CC1CCN(c2cc(-c3ccccc3O)nnc2N)CCN1c1ccnc(C#CCN2CCCCC2)c1]

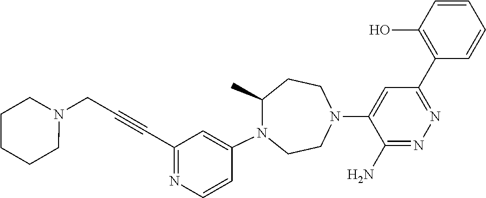 [SMILES: C[C@H]1CCN(c2cc(-c3ccccc3O)nnc2N)CCN1c1ccnc(C#CCN2CCCCC2)c1]

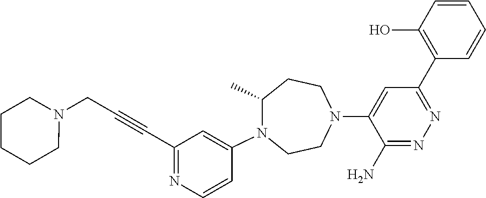 [SMILES: C[C@@H]1CCN(c2cc(-c3ccccc3O)nnc2N)CCN1c1ccnc(C#CCN2CCCCC2)c1]

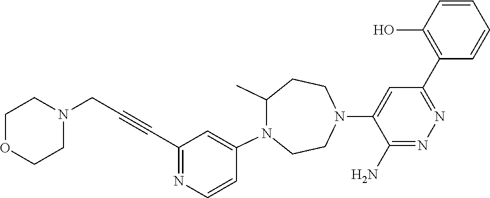 [SMILES: CC1CCN(c2cc(-c3ccccc3O)nnc2N)CCN1c1ccnc(C#CCN2CCOCC2)c1]

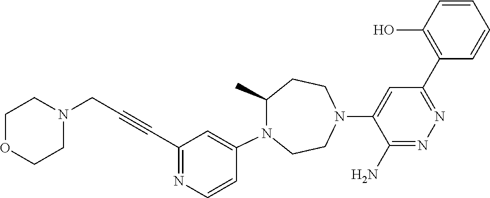 [SMILES: C[C@H]1CCN(c2cc(-c3ccccc3O)nnc2N)CCN1c1ccnc(C#CCN2CCOCC2)c1]

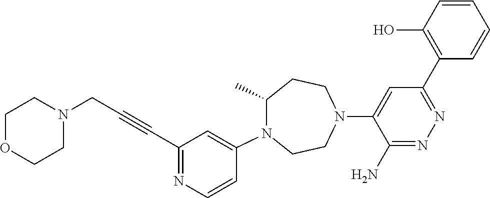 [SMILES: C[C@@H]1CCN(c2cc(-c3ccccc3O)nnc2N)CCN1c1ccnc(C#CCN2CCOCC2)c1]

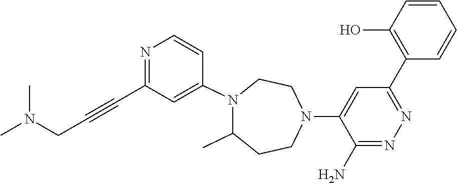 [SMILES: CC1CCN(c2cc(-c3ccccc3O)nnc2N)CCN1c1ccnc(C#CCN(C)C)c1]

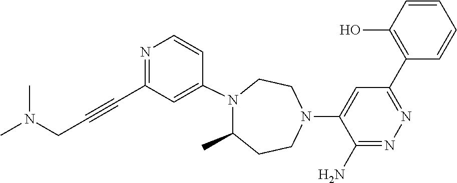 [SMILES: C[C@@H]1CCN(c2cc(-c3ccccc3O)nnc2N)CCN1c1ccnc(C#CCN(C)C)c1]

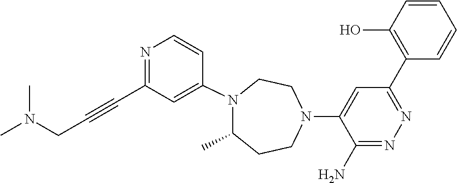 [SMILES: C[C@H]1CCN(c2cc(-c3ccccc3O)nnc2N)CCN1c1ccnc(C#CCN(C)C)c1]

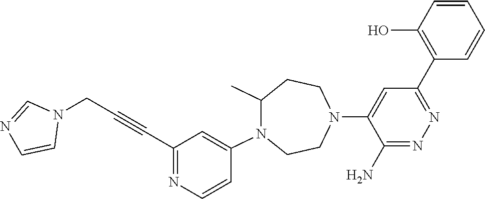 [SMILES: CC1CCN(c2cc(-c3ccccc3O)nnc2N)CCN1c1ccnc(C#CCn2ccnc2)c1]

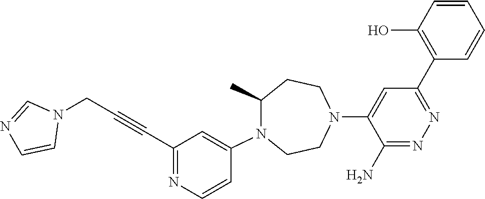 [SMILES: C[C@H]1CCN(c2cc(-c3ccccc3O)nnc2N)CCN1c1ccnc(C#CCn2ccnc2)c1]

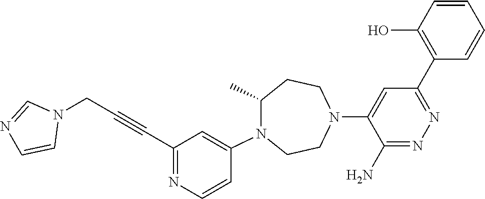 [SMILES: C[C@@H]1CCN(c2cc(-c3ccccc3O)nnc2N)CCN1c1ccnc(C#CCn2ccnc2)c1]

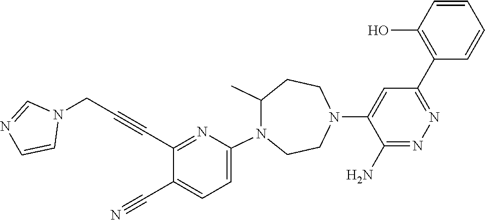 [SMILES: CC1CCN(c2cc(-c3ccccc3O)nnc2N)CCN1c1ccc(C#N)c(C#CCn2ccnc2)n1]

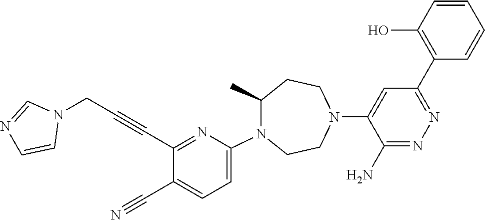 [SMILES: C[C@H]1CCN(c2cc(-c3ccccc3O)nnc2N)CCN1c1ccc(C#N)c(C#CCn2ccnc2)n1]

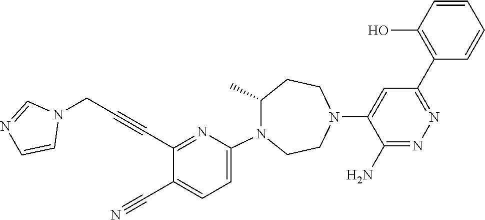 [SMILES: C[C@@H]1CCN(c2cc(-c3ccccc3O)nnc2N)CCN1c1ccc(C#N)c(C#CCn2ccnc2)n1]

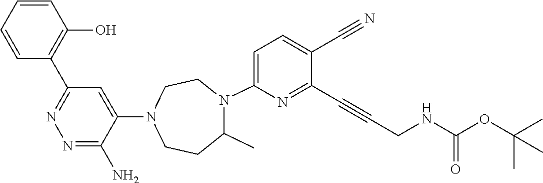 [SMILES: CC1CCN(c2cc(-c3ccccc3O)nnc2N)CCN1c1ccc(C#N)c(C#CCNC(=O)OC(C)(C)C)n1]

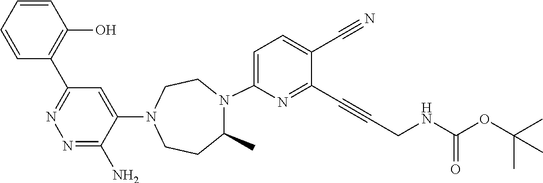 [SMILES: C[C@H]1CCN(c2cc(-c3ccccc3O)nnc2N)CCN1c1ccc(C#N)c(C#CCNC(=O)OC(C)(C)C)n1]

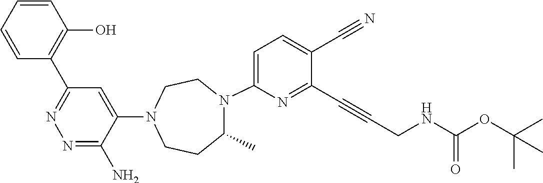 [SMILES: C[C@@H]1CCN(c2cc(-c3ccccc3O)nnc2N)CCN1c1ccc(C#N)c(C#CCNC(=O)OC(C)(C)C)n1]

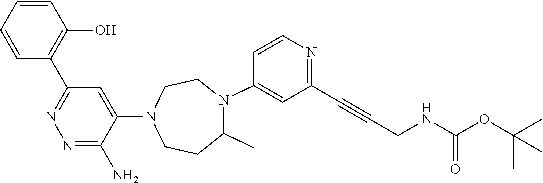 [SMILES: CC1CCN(c2cc(-c3ccccc3O)nnc2N)CCN1c1ccnc(C#CCNC(=O)OC(C)(C)C)c1]

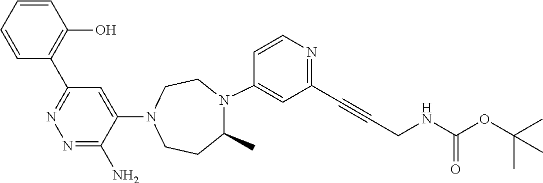 [SMILES: C[C@H]1CCN(c2cc(-c3ccccc3O)nnc2N)CCN1c1ccnc(C#CCNC(=O)OC(C)(C)C)c1]

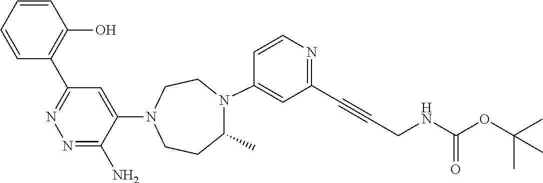 [SMILES: C[C@@H]1CCN(c2cc(-c3ccccc3O)nnc2N)CCN1c1ccnc(C#CCNC(=O)OC(C)(C)C)c1]

In some embodiments, this disclosure provides for a method for modulating or degrading protein which is expressed from the SMARCA2 gene, which method comprises contacting the protein with an effective amount of a compound of Formula I, IA, II, II-1, II-2, II-3, IIa, IIb, IIc, IIc-1, IIc-2, IIc-3, III-E, IIIb-E, I′, II′, IIa′, IIa′-1, IIa′-2, IIb′, IIc′, IId′, III′-E, IIIa′-E, IIIb′-E, III′-Z, IIIa′-Z, or IIIb′-Z, under conditions wherein the protein which is expressed from the SMARCA2 gene is modulated or degraded.

In some embodiments, this disclosure provides for a method for modulating or degrading protein which is expressed from the SMARCA4 gene, which method comprises contacting the protein with an effective amount of a compound of Formula I, IA, II, II-1, II-2, II-3, IIa, IIb, IIc, IIc-1, IIc-2, IIc-3, III-E, IIIb-E, I′, II′, IIa′, IIa′-1, IIa′-2, IIb′, IIc′, IId′, III′-E, IIIa′-E, IIIb′-E, III′-Z, IIIa′-Z, or IIIb′-Z, under conditions wherein the protein which is expressed from the SMARCA4 gene is modulated or degraded.

In some embodiments, there is provided a method to modulate or degrade protein which is expressed from the SMARCA2 gene in a subject, which method comprises administering to said subject an effective amount of a compound of formula I, IA, II, II-1, II-2, II-3, IIa, IIb, IIc, IIc-1, IIc-2, IIc-3, III-E, IIIb-E, I′, II′, IIa′, IIa′-1, IIa′-2, IIb′, IIc′, IId′, III′-E, IIIa′-E, IIIb′-E, III′-Z, IIIa′-Z, or IIIb′-Z, or a pharmaceutical composition comprising a pharmaceutically acceptable excipient and an effective amount of a compound of formula I, IA, II, II-1, II-2, II-3, IIa, IIb, IIc, IIc-1, IIc-2, IIc-3, III-E, IIIb-E, I′, II′, IIa′, IIa′-1, IIa′-2, IIb′, IIc′, IId′, III′-E, IIIa′-E, IIIb′-E, III′-Z, IIIa′-Z, or IIIb′-Z.

In some embodiments, there is provided a method to modulate or degrade protein which is expressed from the SMARCA4 gene in a subject, which method comprises administering to said subject an effective amount of a compound of formula I, IA, II, II-1, II-2, II-3, IIa, IIb, IIc, IIc-1, IIc-2, IIc-3, III-E, IIIb-E, I′, II′, IIa′, IIa′-1, IIa′-2, IIb′, IIc′, IId′, III′-E, IIIa′-E, IIIb′-E, III′-Z, IIIa′-Z, or IIIb′-Z, or a pharmaceutical composition comprising a pharmaceutically acceptable excipient and an effective amount of a compound of formula I, IA, II, II-1, II-2, II-3, IIa, IIb, IIc, IIc-1, IIc-2, IIc-3, III-E, IIIb-E, I′, II′, IIa′, IIa′-1, IIa′-2, IIb′, IIc′, IId′, III′-E, IIIa′-E, IIIb′-E, III′-Z, IIIa′-Z, or IIIb′-Z.

In some embodiments, there is provided a method for treating cancer in a subject in need thereof, which method comprises selecting a subject whose cancer is mediated at least in part by SMARCA2 and administering to said subject an effective amount of a compound of formula I, IA, II, II-1, II-2, II-3, IIa, IIb, IIc, IIc-1, IIc-2, IIc-3, III-E, IIIb-E, I′, II′, IIa′, IIa′-1, IIa′-2, IIb′, IIc′, IId′, III′-E, IIIa′-E, IIIb′-E, III′-Z, IIIa′-Z, or IIIb′-Z, or an effective amount of a pharmaceutical composition comprising a pharmaceutically acceptable excipient and an effective amount of a compound of formula I, IA, II, II-1, II-2, II-3, IIa, IIb, IIc, IIc-1, IIc-2, IIc-3, III-E, IIIb-E, I′, II′, IIa′, IIa′-1, IIa′-2, IIb′, IIc′, IId′, III′-E, IIIa′-E, IIIb′-E, III′-Z, IIIa′-Z, or IIIb′-Z.

In some embodiments, there is provided a method for treating cancer in a subject in need thereof, which method comprises selecting a subject whose cancer is mediated at least in part by SMARCA4 and administering to said subject an effective amount of a compound of formula I, IA, II, II-1, II-2, II-3, IIa, IIb, IIc, IIc-1, IIc-2, IIc-3, III-E, IIIb-E, I′, II′, IIa′, IIa′-1, IIa′-2, IIb′, IIc′, IId′, III′-E, IIIa′-E, IIIb′-E, III′-Z, IIIa′-Z, or IIIb′-Z, or an effective amount of a pharmaceutical composition comprising a pharmaceutically acceptable excipient and an effective amount of a compound of formula I, IA, II, II-1, II-2, II-3, IIa, IIb, IIc, IIc-1, IIc-2, IIc-3, III-E, IIIb-E, I′, II′, IIa′, IIa′-1, IIa′-2, IIb′, IIc′, IId′, III′-E, IIIa′-E, IIIb′-E, III′-Z, IIIa′-Z, or IIIb′-Z.

### General Synthetic Methods

The compounds described herein can be prepared from readily available starting materials using the following general methods and procedures. It will be appreciated that where typical process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and P. G. M. Wuts, Protecting Groups in Organic Synthesis, Third Edition, Wiley, New York, 1999, and references cited therein.

The starting materials for the following reactions are generally known compounds or can be prepared by known procedures or obvious modifications thereof. For example, many of the starting materials are available from commercial suppliers such as Sigma Aldrich (St. Louis, Missouri, USA), Bachem (Torrance, California, USA), Emka-Chemce (St. Louis, Missouri, USA). Others may be prepared by procedures, or obvious modifications thereof, described in standard reference texts such as Fieser and Fieser's Reagentsfor Organic Synthesis, Volumes 1-15 (John Wiley, and Sons, 2016), Rodd's Chemistry of Carbon Compounds, Volumes 1-5, and Supplementals (Elsevier Science Publishers, 2001), Organic Reactions, Volumes 1-40 (John Wiley, and Sons, 2019), March's Advanced Organic Chemistry, (John Wiley, and Sons, 8^{th} Edition, 2019), and Larock's Comprehensive Organic Transformations (VCH Publishers Inc., 1989).

### Synthesis of Representative Compounds

The general synthesis of the compounds described herein is set forth in the reaction schemes below. Schemes 1, 2, 3, 4, 5, and 6 illustrate general methods for preparing compounds of formula I and I′. In Schemes 1, 2, 3, 4, 5, and 6, substituents L, L¹, L², m, n, R, R⁶, T, X, Y, Y¹, X¹, X², Z, and Ring A are as defined throughout the specification. PG is a protecting group (including, but not limited to, Boc and the like). LG is a suitable coupling partner (including, but not limited to, hydrogen when undertaking a Sonogashira coupling, boronic acid or ester when undertaking a Suzuki coupling, and the like).

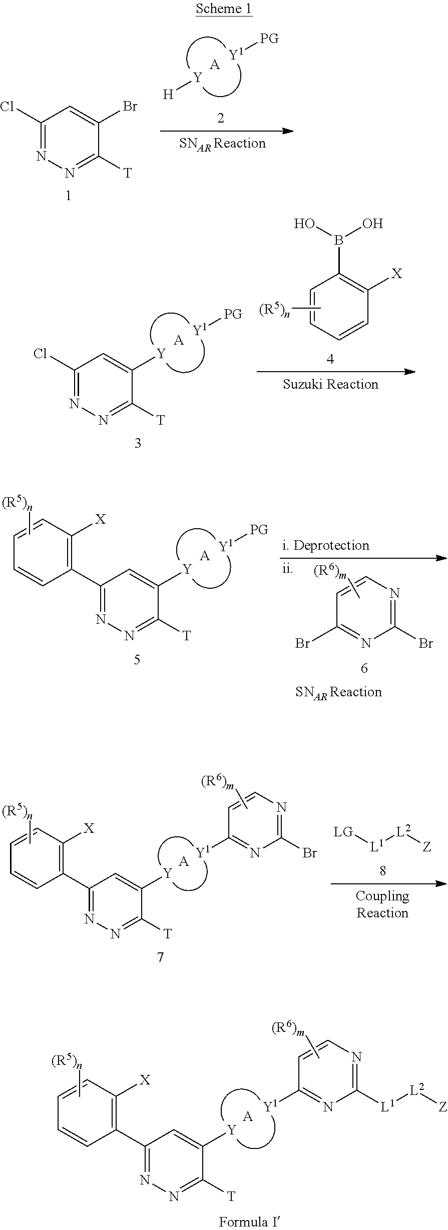

In some embodiments, compounds of formula I′ and sub-formulae thereof are prepared as shown in Scheme 1. In Scheme 1, the first step is a conventional SN_{AR} reaction, wherein at least a stoichiometric amount of a protected Ring A, compound 2, is combined with compound 1 in an inert diluent, such as tetrahydrofuran, dioxane, DMSO, DMF, and the like, typically in the presence of a suitable base, such as diisopropylethylamine, triethylamine, pyridine, potassium carbonate, and the like. The reaction is typically maintained at from 25° C. to 100° C., until it is substantially complete. Conventional workup of the reaction solution can be followed by isolation/purification processes such as crystallization, chromatography, high performance liquid chromatography (HPLC), and the like to provide for compound 3.

In the next step, a conventional Suzuki coupling reaction is performed, wherein at least a stoichiometric amount of aryl boronic acid, compound 4, is combined with compound 3, in an inert diluent, such as tetrahydrofuran, dioxane, toluene, dimethoxyethane, and the like, typically in the presence of a palladium catalyst (e.g., palladium diacetate) and a suitable base, such as diisopropylethylamine, triethylamine, pyridine, potassium carbonate, and the like. The reaction is typically maintained at from 10° C. to 65° C., until it is substantially complete. Conventional workup of the reaction solution can be followed by isolation/purification processes such as crystallization, chromatography, high performance liquid chromatography (HPLC), and the like to provide for compound 5.

In the next step, the protecting group, such as t-butoxycarbonyl (t-BOC), is removed under conventional conditions, depending on the specific protecting group employed. The t-BOC group is illustrative only, and other conventional amino protecting groups, such as benzyl, 9-fluorenylmethoxycarbonyl (Fmoc), benzyloxycarbonyl (Cbz), p-nitrobenzyloxycarbonyl, and the like could be employed. Upon reaction completion, conventional workup of the reaction solution can be followed by isolation/purification processes such as crystallization, chromatography, high performance liquid chromatography (HPLC), and the like.

In the next step, a conventional SNA reaction is performed, wherein at least a stoichiometric amount of the deprotected product from the previous step is combined 2,4-dibromopyrimidine, compound 6, in an inert diluent, such as tetrahydrofuran, dioxane, DMSO, DMF, and the like, typically in the presence of a suitable base, such as diisopropylethylamine, triethylamine, pyridine, potassium carbonate, and the like. The reaction is typically maintained at from 25° C. to 100° C., until it is substantially complete. Conventional workup of the reaction solution can be followed by isolation/purification processes such as crystallization, chromatography, high performance liquid chromatography (HPLC), and the like to provide for compound 7.

In the final step, a conventional coupling reaction, including, but not limited to, a Sonogashira coupling, a Suzuki coupling, and the like, is performed, wherein at least a stoichiometric amount of a suitable coupling partner, compound 8, is combined with compound 7 under conventional coupling reaction conditions well known in the art, including the use of a palladium catalyst (e.g., palladium(II) bis(triphenylphosphine) dichloride, palladium diacetate, and the like), a co-catalyst (e.g., copper (I) iodide and the like), and typically in the presence of a suitable base (e.g., diisopropylethylamine, triethylamine, pyridine, cesium carbonate, and the like). The coupling reaction is typically conducted in an inert solvent, such as toluene, N,N-dimethylformamide, tetrahydrofuran, dioxane, dimethoxyethane, and the like. The reaction is typically conducted at from about 10° C. to about 110° C., for a period of time sufficient for substantial completion of the reaction as evidenced by e.g., thin layer chromatography. Upon reaction completion, conventional workup of the reaction solution can be followed by isolation/purification processes such as crystallization, chromatography, high performance liquid chromatography (HPLC), and the like to provide for compounds of Formula I′.

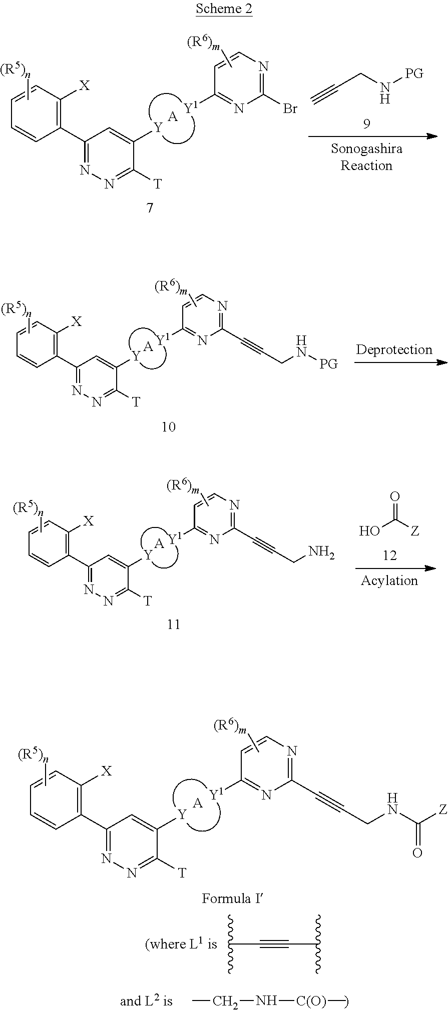

In some embodiments, compounds of formula I′ and sub-formulae thereof are prepared as shown in Scheme 2. In Scheme 2, the first step is a conventional Sonogashira coupling reaction employing at least a stoichiometric amount of protected propargyl amine, compound 9, combined with compound 7 under conventional reaction conditions well known in the art, including the use of palladium (II) bis(triphenylphosphine) dichloride and copper (1) iodide as catalysts, and typically in the presence of a suitable base, such as diisopropylethylamine, triethylamine, pyridine, cesium carbonate, and the like. The reaction is typically conducted in an inert solvent, such as toluene, N,N-dimethylformamide, and the like. The reaction is typically conducted at from about 25° C. to about 110° C., for a period of time sufficient for substantial completion of the reaction as evidenced by e.g., thin layer chromatography. Upon reaction completion, conventional workup of the reaction solution can be followed by isolation/purification processes such as crystallization, chromatography, high performance liquid chromatography (HPLC), and the like to provide for compound 10.

In the next step, the protecting group, such as t-butoxycarbonyl (t-BOC), is removed under conventional conditions, depending on the specific protecting group employed. The t-BOC group is illustrative only, and other conventional amino protecting groups, such as benzyl, 9-fluorenylmethoxycarbonyl (Fmoc), benzyloxycarbonyl (Cbz), p-nitrobenzyloxycarbonyl, and the like could be employed. Upon reaction completion, conventional workup of the reaction solution can be followed by isolation/purification processes such as crystallization, chromatography, high performance liquid chromatography (HPLC), and the like to provide for compound 11.

In the final step, at least a stoichiometric amount of a suitable carboxylic acid, compound 12, is combined with compound 11, under conventional amide bond formation reaction conditions well known in the art, including the use of N,N-dicyclohexylcarbodiimide (DCC) as an activation agent for the carboxyl group. Other activation agents are well known in the art. The reaction is typically conducted in an inert solvent, such as chloroform, methylene chloride, toluene, N,N-dimethylformamide, and the like. The reaction is typically conducted at from about 0° C. to about 30° C., for a period of time sufficient for substantial completion of the reaction as evidenced by e.g., thin layer chromatography. Upon reaction completion, conventional workup of the reaction solution can be followed by isolation/purification processes such as crystallization, chromatography, high performance liquid chromatography (HPLC), and the like to provide for compounds of Formula I′.

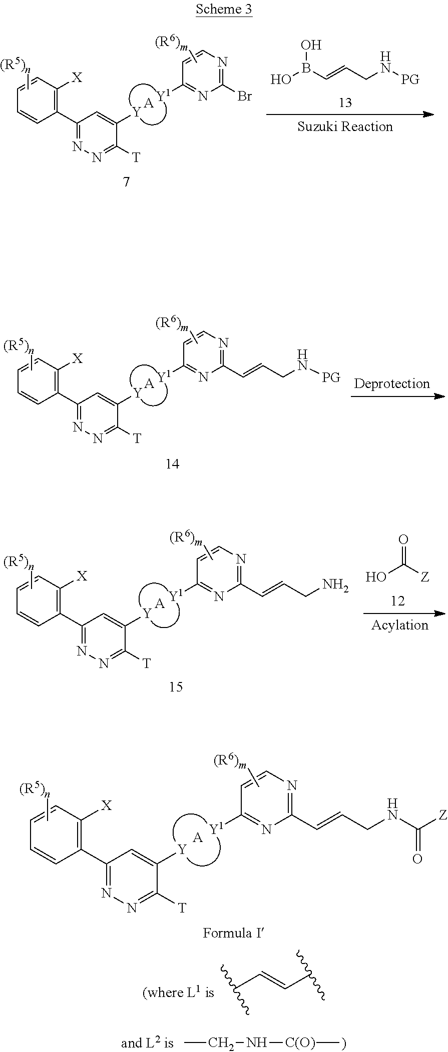

In some embodiments, compounds of formula I′ and sub-formulae thereof are prepared as shown in Scheme 3. In Scheme 3, the first step is a conventional Suzuki coupling reaction, wherein at least a stoichiometric amount of protected amino vinylboronic acid, compound 13, is combined with compound 7, in an inert diluent, such as tetrahydrofuran, dioxane, toluene, dimethoxyethane, and the like, typically in the presence of a palladium catalyst (e.g, palladium diacetate), and a suitable base, such as diisopropylethylamine, triethylamine, pyridine, potassium carbonate, and the like. The reaction is typically maintained at from 10° C. to 65° C., until it is substantially complete. Conventional workup of the reaction solution can be followed by isolation/purification processes such as crystallization, chromatography, high performance liquid chromatography (HPLC), and the like to provide for compound 14.

In the next step, the protecting group, such as t-butoxycarbonyl (t-BOC), is removed under conventional conditions, depending on the specific protecting group employed. The t-BOC group is illustrative only, and other conventional amino protecting groups, such as benzyl, 9-fluorenylmethoxycarbonyl (Fmoc), benzyloxycarbonyl (Cbz), p-nitrobenzyloxycarbonyl, and the like could be employed. Upon reaction completion, conventional workup of the reaction solution can be followed by isolation/purification processes such as crystallization, chromatography, high performance liquid chromatography (HPLC), and the like to provide for compound 15.

In the final step, at least a stoichiometric amount of a suitable carboxylic acid, compound 12, is combined with compound 15, under conventional amide bond formation reaction conditions well known in the art, including the use of N,N-dicyclohexylcarbodiimide (DCC) as an activation agent for the carboxyl group. Other activation agents are well known in the art. The reaction is typically conducted in an inert solvent, such as chloroform, methylene chloride, toluene, N,N-dimethylformamide, and the like. The reaction is typically conducted at from about 0° C. to about 30° C., for a period of time sufficient for substantial completion of the reaction as evidenced by e.g., thin layer chromatography. Upon reaction completion, conventional workup of the reaction solution can be followed by isolation/purification processes such as crystallization, chromatography, high performance liquid chromatography (HPLC), and the like to provide for compounds of Formula I′.

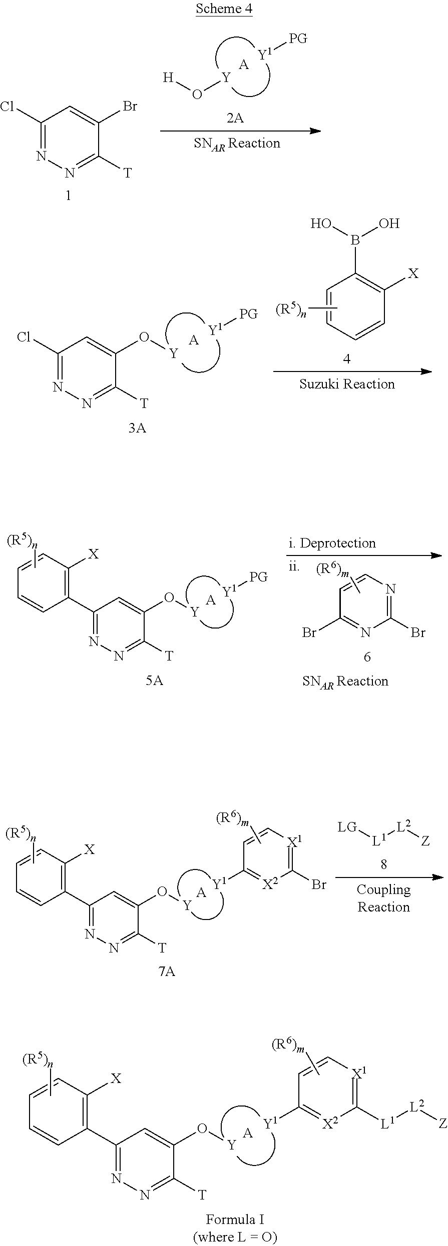

In some embodiments, compounds of formula I and sub-formulae thereof are prepared as shown in Scheme 4. In Scheme 4, the first step is a conventional SN_{AR} reaction, wherein at least a stoichiometric amount of a protected Ring A, compound 2A, is combined with compound 1 in an inert diluent, such as tetrahydrofuran, dioxane, DMSO, DMF, and the like, typically in the presence of a suitable base, such as sodium hydride, potassium carbonate, cesium carbonate, and the like. The reaction is typically maintained at from 25° C. to 100° C., until it is substantially complete. Conventional workup of the reaction solution can be followed by isolation/purification processes such as crystallization, chromatography, high performance liquid chromatography (HPLC), and the like to provide for compound 3A.

In the next step, a conventional Suzuki coupling reaction is performed, wherein at least a stoichiometric amount of aryl boronic acid, compound 4, is combined with compound 3A, in an inert diluent, such as tetrahydrofuran, dioxane, toluene, dimethoxyethane, and the like, typically in the presence of a palladium catalyst (e.g, palladium diacetate) and a suitable base, such as diisopropylethylamine, triethylamine, pyridine, potassium carbonate, and the like. The reaction is typically maintained at from 10° C. to 65° C., until it is substantially complete. Conventional workup of the reaction solution can be followed by isolation/purification processes such as crystallization, chromatography, high performance liquid chromatography (HPLC), and the like to provide for compound 5A.

In the next step, the protecting group, such as t-butoxycarbonyl (t-BOC), is removed under conventional conditions, depending on the specific protecting group employed. The t-BOC group is illustrative only, and other conventional amino protecting groups, such as benzyl, 9-fluorenylmethoxycarbonyl (Fmoc), benzyloxycarbonyl (Cbz), p-nitrobenzyloxycarbonyl, and the like could be employed. Upon reaction completion, conventional workup of the reaction solution can be followed by isolation/purification processes such as crystallization, chromatography, high performance liquid chromatography (HPLC), and the like.

In the next step, a conventional SNA reaction is performed, wherein at least a stoichiometric amount of the deprotected product from the previous step is combined 2,4-dibromopyrimidine, compound 6, in an inert diluent, such as tetrahydrofuran, dioxane, DMSO, DMF, and the like, typically in the presence of a suitable base, such as diisopropylethylamine, triethylamine, pyridine, potassium carbonate, and the like. The reaction is typically maintained at from 25° C. to 100° C., until it is substantially complete. Conventional workup of the reaction solution can be followed by isolation/purification processes such as crystallization, chromatography, high performance liquid chromatography (HPLC), and the like to provide for compound 7A.

In the final step, a conventional coupling reaction, including, but not limited to, a Sonogashira coupling, a Suzuki coupling, and the like, is performed, wherein at least a stoichiometric amount of a suitable coupling partner, compound 8, is combined with compound 7 under conventional coupling reaction conditions well known in the art, including the use of a palladium catalyst (e.g., palladium(II) bis(triphenylphosphine) dichloride, palladium diacetate, and the like), a co-catalyst (e.g., copper (I) iodide and the like), and typically in the presence of a suitable base (e.g., diisopropylethylamine, triethylamine, pyridine, cesium carbonate, and the like). The coupling reaction is typically conducted in an inert solvent, such as toluene, N,N-dimethylformamide, tetrahydrofuran, dioxane, dimethoxyethane, and the like. The reaction is typically conducted at from about 10° C. to about 110° C., for a period of time sufficient for substantial completion of the reaction as evidenced by e.g., thin layer chromatography. Upon reaction completion, conventional workup of the reaction solution can be followed by isolation/purification processes such as crystallization, chromatography, high performance liquid chromatography (HPLC), and the like to provide for compounds of Formula I.

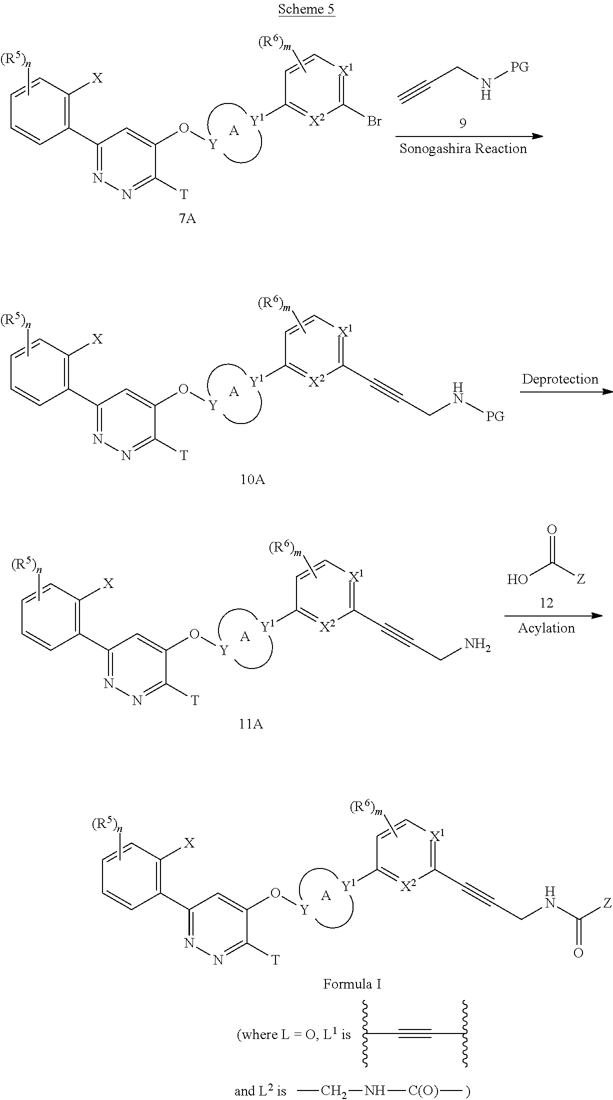

In some embodiments, compounds of formula I and sub-formulae thereof are prepared as shown in Scheme 5. In Scheme 5, the first step is a conventional Sonogashira coupling reaction employing at least a stoichiometric amount of protected propargyl amine, compound 9, combined with compound 7A under conventional reaction conditions well known in the art, including the use of palladium (II) bis(triphenylphosphine) dichloride and copper (I) iodide as catalysts, and typically in the presence of a suitable base, such as diisopropylethylamine, triethylamine, pyridine, cesium carbonate, and the like. The reaction is typically conducted in an inert solvent, such as toluene, N,N-dimethylformamide, and the like. The reaction is typically conducted at from about 25° C. to about 110° C., for a period of time sufficient for substantial completion of the reaction as evidenced by e.g., thin layer chromatography. Upon reaction completion, conventional workup of the reaction solution can be followed by isolation/purification processes such as crystallization, chromatography, high performance liquid chromatography (HPLC), and the like to provide for compound 10A.

In the next step, the protecting group, such as t-butoxycarbonyl (t-BOC), is removed under conventional conditions, depending on the specific protecting group employed. The t-BOC group is illustrative only, and other conventional amino protecting groups, such as benzyl, 9-fluorenylmethoxycarbonyl (Fmoc), benzyloxycarbonyl (Cbz), p-nitrobenzyloxycarbonyl, and the like could be employed. Upon reaction completion, conventional workup of the reaction solution can be followed by isolation/purification processes such as crystallization, chromatography, high performance liquid chromatography (HPLC), and the like to provide for compound 11A.

In the final step, at least a stoichiometric amount of a suitable carboxylic acid, compound 12, is combined with compound 11A, under conventional amide bond formation reaction conditions well known in the art, including the use of N,N-dicyclohexylcarbodiimide (DCC) as an activation agent for the carboxyl group. Other activation agents are well known in the art. The reaction is typically conducted in an inert solvent, such as chloroform, methylene chloride, toluene, N,N-dimethylformamide, and the like. The reaction is typically conducted at from about 0° C. to about 30° C., for a period of time sufficient for substantial completion of the reaction as evidenced by e.g., thin layer chromatography. Upon reaction completion, conventional workup of the reaction solution can be followed by isolation/purification processes such as crystallization, chromatography, high performance liquid chromatography (HPLC), and the like to provide for compounds of Formula I.

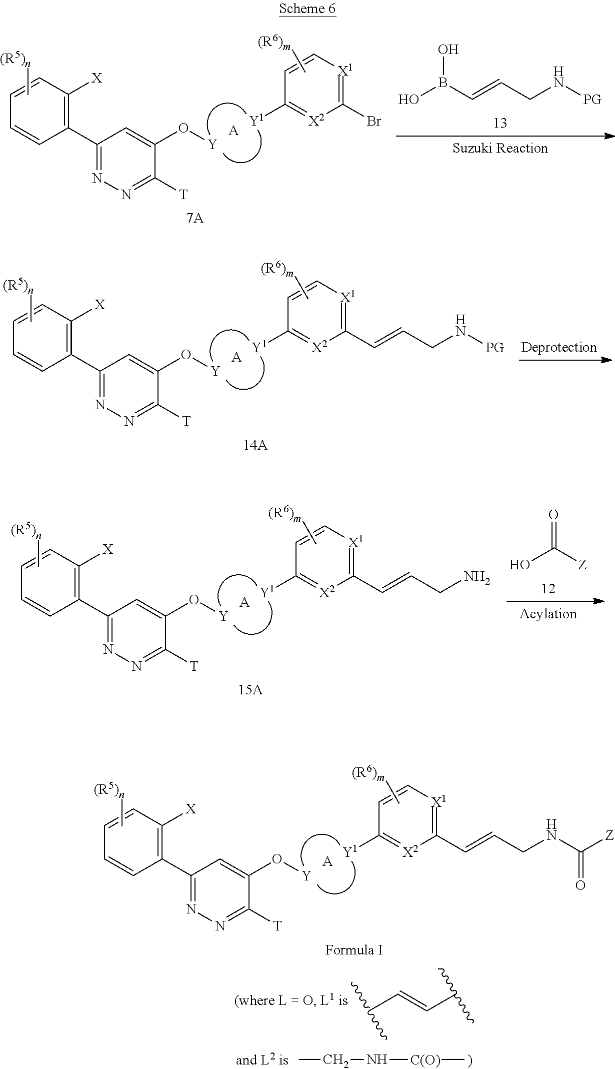

In some embodiments, compounds of formula I and sub-formulae thereof are prepared as shown in Scheme 3′. In Scheme 3′, the first step is a conventional Suzuki coupling reaction, wherein at least a stoichiometric amount of protected amino vinylboronic acid, compound 13, is combined with compound 7A, in an inert diluent, such as tetrahydrofuran, dioxane, toluene, dimethoxyethane, and the like, typically in the presence of a palladium catalyst (e.g, palladium diacetate), and a suitable base, such as diisopropylethylamine, triethylamine, pyridine, potassium carbonate, and the like. The reaction is typically maintained at from 10° C. to 65° C., until it is substantially complete. Conventional workup of the reaction solution can be followed by isolation/purification processes such as crystallization, chromatography, high performance liquid chromatography (HPLC), and the like to provide for compound 14A.

In the next step, the protecting group, such as t-butoxycarbonyl (t-BOC), is removed under conventional conditions, depending on the specific protecting group employed. The t-BOC group is illustrative only, and other conventional amino protecting groups, such as benzyl, 9-fluorenylmethoxycarbonyl (Fmoc), benzyloxycarbonyl (Cbz), p-nitrobenzyloxycarbonyl, and the like could be employed. Upon reaction completion, conventional workup of the reaction solution can be followed by isolation/purification processes such as crystallization, chromatography, high performance liquid chromatography (HPLC), and the like to provide for compound 15A.

In the final step, at least a stoichiometric amount of a suitable carboxylic acid, compound 12, is combined with compound 15A, under conventional amide bond formation reaction conditions well known in the art, including the use of N,N-dicyclohexylcarbodiimide (DCC) as an activation agent for the carboxyl group. Other activation agents are well known in the art. The reaction is typically conducted in an inert solvent, such as chloroform, methylene chloride, toluene, N,N-dimethylformamide, and the like. The reaction is typically conducted at from about 0° C. to about 30° C., for a period of time sufficient for substantial completion of the reaction as evidenced by e.g., thin layer chromatography. Upon reaction completion, conventional workup of the reaction solution can be followed by isolation/purification processes such as crystallization, chromatography, high performance liquid chromatography (HPLC), and the like to provide for compounds of Formula 1.

Other starting materials used herein are either well known in the art, commercially available, or can be prepared by conventional synthetic methods.

### Methods

In one embodiment, the compounds and compositions described herein are useful in methods for treating a SMARCA2 dependent disease or disorder or a disease or disorder that is mediated, at least in part by, SMARCA2. The methods comprise administering to a subject suffering from a SMARCA2 dependent disease or disorder an effective amount of a compound, or a pharmaceutically acceptable salt, solvate, stereoisomer, or tautomer thereof or a pharmaceutical composition comprising said compound, or a pharmaceutically acceptable salt, solvate, stereoisomer, or tautomer thereof as described herein.

In one embodiment, there is provided a compound, or a pharmaceutically acceptable salt, solvate, stereoisomer, or tautomer thereof or a pharmaceutical composition comprising said compound, or a pharmaceutically acceptable salt, solvate, stereoisomer, or tautomer thereof as described herein for use in treating an SMARCA2 dependent disease or disorder.

In one embodiment, the method relates a compound, or a pharmaceutically acceptable salt, solvate, stereoisomer, or tautomer thereof or a pharmaceutical composition comprising said compound, or a pharmaceutically acceptable salt, solvate, stereoisomer, or tautomer thereof as described herein for use in manufacture of a medicament for reducing levels of protein which is expressed from the SMARCA2 gene, where reduction of such protein levels treats or ameliorates the diseases or disorder.

In one embodiment, the methods described herein comprise use of a prodrug of the compounds described herein.

In one embodiment, the method relates a compound, or a pharmaceutically acceptable salt, solvate, stereoisomer, or tautomer thereof or a pharmaceutical composition comprising said compound, or a pharmaceutically acceptable salt, solvate, stereoisomer, or tautomer thereof as described herein for use as described herein, wherein the degradation of protein which is expressed from the SMARCA2 gene at 1 μM concentration of the compounds described herein is in the range of about 25%-99%. The degradation of the protein which is expressed from the SMARCA2 gene is measured by the assay described in the biological example. In some embodiments, the degradation of the protein which is expressed from the SMARCA2 gene is from about 25% to about 50%, from about 45% to about 70%, from about 65% to about 90% or from about 75% to about 99%. In some embodiments, the degradation of the protein which is expressed from the SMARCA2 gene is from about 25% to about 35%, from about 35% to about 45%, from about 45% to about 55%, from about 55% to about 65%, from about 65% to about 75%, from about 75% to about 85%, from about 85% to about 99%. In some embodiments, the degradation of the protein which is expressed from the SMARCA2 gene is more than 60%. In some embodiments, the degradation of the protein which is expressed from the SMARCA2 gene is more than 70%. In some embodiments, the degradation of the protein which is expressed from the SMARCA2 gene is more than 80%. In some embodiments, the degradation of the protein which is expressed from the SMARCA2 gene is more than 90%.

The compounds and compositions described herein are useful in treating SMARCA2 dependent diseases or disorders such as liposarcoma, glioblastoma, bladder cancer, adrenocortical cancer, multiple myeloma, colorectal cancer, non-small cell lung cancer, Human Papilloma Virus-associated cervical, oropharyngeal, penis, anal, thyroid, or vaginal cancer or Epstein-Barr Virus-associated nasopharyngeal carcinoma, gastric cancer, rectal cancer, thyroid cancer, Hodgkin lymphoma or diffuse large B-cell lymphoma. The cancer may be selected from prostate cancer, breast carcinoma, lymphomas, leukemia, myeloma, bladder carcinoma, colon cancer, cutaneous melanoma, hepatocellular carcinoma, endometrial cancer, ovarian cancer, cervical cancer, lung cancer, renal cancer, glioblastoma multiform, glioma, thyroid cancer, parathyroid tumor, nasopharyngeal cancer, tongue cancer, pancreatic cancer, esophageal cancer, cholangiocarcinoma, gastric cancer, soft tissue sarcomas, rhabdomyosarcoma (RMS), synovial sarcoma, osteosarcoma, rhabdoid cancers, cancer for which the immune response is deficient, an immunogenic cancer, and Ewing's sarcoma. In one embodiment, the SMARCA2-dependent disease or disorder is a disease or disorder is selected from non-small cell lung cancer (NSCLC), melanoma, triple-negative breast cancer (TNBC), nasopharyngeal cancer (NPC), microsatellite stable colorectal cancer (mssCRC), thymoma, carcinoid, and gastrointestinal stromal tumor (GIST). In another embodiment, the cancer is selected from non-small cell lung cancer (NSCLC), melanoma, triple-negative breast cancer (TNBC), nasopharyngeal cancer (NPC), microsatellite stable colorectal cancer (mssCRC), thymoma, carcinoid, acute myclogenous leukemia, and gastrointestinal stromal tumor (GIST). In another embodiment, the SMARCA2-dependent disease or disorder is a disease or disorder is selected from non-small cell lung cancer (NSCLC), melanoma, triple-negative breast cancer (TNBC), nasopharyngeal cancer (NPC), and microsatellite stable colorectal cancer (mssCRC).

The compounds of the disclosure can be administered in effective amounts to treat or prevent a disorder and/or prevent the development thereof in subjects.

In general, methods of using the compounds of the present application comprise administering to a subject in need thereof a therapeutically effective amount of a compound as described herein.

In certain embodiments, compounds as described herein are useful in the treatment of proliferative disorders (e.g., cancer, benign neoplasms, inflammatory disease, and autoimmune diseases). In certain embodiments, according to the methods of treatment of the present application, levels of cell proteins of interest, e.g., pathogenic and oncogenic proteins are modulated, or their growth is inhibited or the proteins are degraded by contacting said cells with an compound or composition, as described herein. In other embodiments, the compounds are useful in treating cancer.

Thus, in another aspect of the application, methods for the treatment of cancer are provided comprising administering a therapeutically effective amount of compound or composition, as described herein, to a subject in need thereof. In certain embodiments, a method for the treatment of cancer is provided comprising administering a therapeutically effective amount of a compound, or a pharmaceutical composition comprising a compound as described herein to a subject in need thereof, in such amounts and for such time as is necessary to achieve the desired result. In some embodiments, the compounds of present application are administered orally or intravenously. In certain embodiments of the present application a “therapeutically effective amount” of the compound or pharmaceutical composition is that amount effective for killing or inhibiting the growth of tumor cells. The compounds and compositions, according to the method of the present application, may be administered using any amount and any route of administration effective for killing or inhibiting the growth of tumor cells. Thus, the expression “amount effective to kill or inhibit the growth of tumor cells,” as used herein, refers to a sufficient amount of agent to kill or inhibit the growth of tumor cells. The exact amount required will vary from subject to subject, depending on the species, age, and general condition of the subject, the severity of the disease, the particular anticancer agent, its mode of administration, and the like. In certain embodiments of the present application a “therapeutically effective amount” of the-compound or pharmaceutical composition described herein is that amount effective for reducing the levels of target proteins. In certain embodiments of the present application a “therapeutically effective amount” of the compound or pharmaceutical composition is that amount effective to kill or inhibit the growth of skin cells.

In certain embodiments, the method involves the administration of a therapeutically effective amount of the compound or a pharmaceutically acceptable derivative thereof to a subject (including, but not limited to a human or other mammal in need of it. In certain embodiments, the compounds or compositions described herein are useful for the treatment of cancer (including, but not limited to, glioblastoma, retinoblastoma, breast cancer, cervical cancer, colon and rectal cancer, leukemia, lymphoma, lung cancer (including, but not limited to small cell lung cancer), melanoma and/or skin cancer, multiple myeloma, non-Hodgkin's lymphoma, ovarian cancer, pancreatic cancer, prostate cancer and gastric cancer, bladder cancer, uterine cancer, kidney cancer, testicular cancer, stomach cancer, brain cancer, liver cancer, or esophageal cancer).

In certain embodiments, the compounds or compositions described herein are useful in the treatment of cancers and other proliferative disorders, including, but not limited to breast cancer, cervical cancer, colon and rectal cancer, leukemia, lung cancer, melanoma, multiple myeloma, non-Hodgkin's lymphoma, ovarian cancer, pancreatic cancer, prostate cancer, and gastric cancer. In certain embodiments, compounds or compositions described herein are active against solid tumors.

Additionally, the present application provides pharmaceutically acceptable derivatives of the compounds, and methods of treating a subject using these compounds, pharmaceutical compositions thereof, or either of these in combination with one or more additional therapeutic agents.

Another aspect of the application relates to a method of treating or lessening the severity of a disease or condition associated with a proliferation disorder in a patient, said method comprising a step of administering to said patient, a compound of Formula 1 or a composition comprising said compound.

It will be appreciated that the compounds and compositions, according to the method of the present application, may be administered using any amount and any route of administration effective for the treatment of cancer and/or disorders associated with cell hyperproliferation. For example, when using the compounds for the treatment of cancer, the expression “effective amount” as used herein, refers to a sufficient amount of agent to inhibit proliferation, or refers to a sufficient amount to reduce the effects of cancer. The exact amount required will vary from subject to subject, depending on the species, age, and general condition of the subject, the severity of the diseases, the particular anticancer agent, its mode of administration, and the like.

The present application provides methods for the treatment of a proliferative disorder in a subject in need thereof by administering to a subject in need of such treatment, a therapeutically effective amount of a compound of the present application, or a pharmaceutically acceptable salt, solvate, stereoisomer, or tautomer thereof. The proliferative disorder can be cancer or a precancerous condition. The present application further provides the use of a compound of the present application, or a pharmaceutically acceptable salt, salt, solvate, stereoisomer, or tautomer thereof, for the preparation of a medicament useful for the treatment of a proliferative disorder.

The present application also provides methods of protecting against a proliferative disorder in a subject in need thereof by administering a therapeutically effective amount of compound of the present application, or a pharmaceutically acceptable salt, salt, solvate, stereoisomer, or tautomer thereof, to a subject in need of such treatment. The proliferative disorder can be cancer or a precancerous condition. The present application also provides the use of compound of the present application, or a pharmaceutically acceptable salt, salt, solvate, stereoisomer, or tautomer thereof, for the preparation of a medicament useful for the prevention of a proliferative disorder.

As used herein, the term “proliferative disorder” refers to conditions in which unregulated or abnormal growth, or both, of cells can lead to the development of an unwanted condition or disease, which may or may not be cancerous. Exemplary proliferative disorders of the application encompass a variety of conditions wherein cell division is deregulated. Exemplary proliferative disorder include, but are not limited to, neoplasms, benign tumors, malignant tumors, pre-cancerous conditions, in situ tumors, encapsulated tumors, metastatic tumors, liquid tumors, solid tumors, immunological tumors, hematological tumors, cancers, carcinomas, leukemias, lymphomas, sarcomas, and rapidly dividing cells. The term “rapidly dividing cell” as used herein is defined as any cell that divides at a rate that exceeds or is greater than what is expected or observed among neighboring or juxtaposed cells within the same tissue. A proliferative disorder includes a precancer or a precancerous condition. A proliferative disorder includes cancer. The methods provided herein are used to treat or alleviate a symptom of cancer. The term “cancer” includes solid tumors, as well as, hematologic tumors and/or malignancies. A “precancer cell” or “precancerous cell” is a cell manifesting a proliferative disorder that is a precancer or a precancerous condition. A “cancer cell” or “cancerous cell” is a cell manifesting a proliferative disorder that is a cancer. Any reproducible means of measurement may be used to identify cancer cells or precancerous cells. Cancer cells or precancerous cells can be identified by histological typing or grading of a tissue sample (e.g., a biopsy sample). Cancer cells or precancerous cells can be identified through the use of appropriate molecular markers.

Exemplary non-cancerous conditions or disorders include, but are not limited to, rheumatoid arthritis; inflammation; autoimmune disease; lymphoproliferative conditions; acromegaly; rheumatoid spondylitis; osteoarthritis; gout, other arthritic conditions; sepsis; septic shock; endotoxic shock; gram-negative sepsis; toxic shock syndrome; asthma; adult respiratory distress syndrome; chronic obstructive pulmonary disease; chronic pulmonary inflammation; inflammatory bowel disease; Crohn's disease; psoriasis; eczema; ulcerative colitis; pancreatic fibrosis; hepatic fibrosis; acute and chronic renal disease; irritable bowel syndrome; pyresis; restenosis; cerebral malaria; stroke and ischemic injury; neural trauma; Alzheimer's disease; Huntington's disease; Parkinson's disease; acute and chronic pain; allergic rhinitis; allergic conjunctivitis; chronic heart failure; acute coronary syndrome; cachexia; malaria; leprosy; leishmaniasis; Lyme disease; Reiter's syndrome; acute synovitis; muscle degeneration, bursitis; tendonitis; tenosynovitis; herniated, ruptures, or prolapsed intervertebral disk syndrome; osteopetrosis; thrombosis; restenosis; silicosis; pulmonary sarcoidosis; bone resorption diseases, such as osteoporosis; graft-versus-host reaction; Multiple Sclerosis; lupus; fibromyalgia; AIDS and other viral diseases such as Herpes Zoster, Herpes Simplex I or II, influenza virus and cytomegalovirus; and diabetes mellitus.

Exemplary cancers include, but are not limited to, adrenocortical carcinoma, AIDS-related cancers, AIDS-related lymphoma, anal cancer, anorectal cancer, cancer of the anal canal, appendix cancer, childhood cerebellar astrocytoma, childhood cerebral astrocytoma, basal cell carcinoma, skin cancer (non-melanoma), biliary cancer, extrahepatic bile duct cancer, intrahepatic bile duct cancer, bladder cancer, urinary bladder cancer, bone and joint cancer, osteosarcoma and malignant fibrous histiocytoma, brain cancer, brain tumor, brain stem glioma, cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas/carcinoids, carcinoid tumor, gastrointestinal, nervous system cancer, nervous system lymphoma, central nervous system cancer, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, lymphoid neoplasm, mycosis fungoides, Sezary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin lymphoma, hypopharyngeal cancer, intraocular melanoma, ocular cancer, islet cell tumors (endocrine pancreas), Kaposi Sarcoma, kidney cancer, renal cancer, kidney cancer, laryngeal cancer, acute lymphoblastic leukemia, acute myeloid leukemia, chronic lymphocytic leukemia, chronic myelogenous leukemia, hairy cell leukemia, lip and oral cavity cancer, liver cancer, lung cancer, non-small cell lung cancer, small cell lung cancer, AIDS-related lymphoma, non-Hodgkin lymphoma, primary central nervous system lymphoma, Waldenström macroglobulinemia, medulloblastoma, melanoma, intraocular (eye) melanoma, merkel cell carcinoma, mesothelioma malignant, mesothelioma, metastatic squamous neck cancer, mouth cancer, cancer of the tongue, multiple endocrine neoplasia syndrome, mycosis fungoides, myelodysplastic syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, Ewing family of sarcoma tumors, Kaposi Sarcoma, soft tissue sarcoma, uterine cancer, uterine sarcoma, skin cancer (non-melanoma), skin cancer (melanoma), merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter and other urinary organs, gestational trophoblastic tumor, urethral cancer, endometrial uterine cancer, uterine sarcoma, uterine corpus cancer, vaginal cancer, vulvar cancer, and Wilms' Tumor.

A “proliferative disorder of the hematologic system” is a proliferative disorder involving cells of the hematologic system. A proliferative disorder of the hematologic system can include lymphoma, leukemia, myeloid neoplasms, mast cell neoplasms, myelodysplasia, benign monoclonal gammopathy, lymphomatoid granulomatosis, lymphomatoid papulosis, polycythemia vera, chronic myelocytic leukemia, agnogenic myeloid metaplasia, and essential thrombocythemia. A proliferative disorder of the hematologic system can include hyperplasia, dysplasia, and metaplasia of cells of the hematologic system. The compositions of the present application may be used to treat a cancer selected from the group consisting of a hematologic cancer of the present application or a hematologic proliferative disorder of the present application. A hematologic cancer of the present application can include multiple myeloma, lymphoma (including Hodgkin's lymphoma, non-Hodgkin's lymphoma, childhood lymphomas, and lymphomas of lymphocytic and cutaneous origin), leukemia (including childhood leukemia, hairy-cell leukemia, acute lymphocytic leukemia, acute myelocytic leukemia, chronic lymphocytic leukemia, chronic myelocytic leukemia, chronic myelogenous leukemia, and mast cell leukemia), myeloid neoplasms and mast cell neoplasms.

A “proliferative disorder of the lung” is a proliferative disorder involving cells of the lung. Proliferative disorders of the lung can include all forms of proliferative disorders affecting lung cells. Proliferative disorders of the lung can include lung cancer, a precancer or precancerous condition of the lung, benign growths or lesions of the lung, and malignant growths or lesions of the lung, and metastatic lesions in tissue and organs in the body other than the lung. Compositions of the present application may be used to treat lung cancer or proliferative disorders of the lung. Lung cancer can include all forms of cancer of the lung. Lung cancer can include malignant lung neoplasms, carcinoma in situ, typical carcinoid tumors, and atypical carcinoid tumors. Lung cancer can include small cell lung cancer (“SCLC”), non-small cell lung cancer (“NSCLC”), squamous cell carcinoma, adenocarcinoma, small cell carcinoma, large cell carcinoma, adenosquamous cell carcinoma, and mesothelioma. Lung cancer can include “scar carcinoma”, bronchioalveolar carcinoma, giant cell carcinoma, spindle cell carcinoma, and large cell neuroendocrine carcinoma. Lung cancer can include lung neoplasms having histologic and ultrastructural heterogeneity (e.g., mixed cell types).

Proliferative disorders of the lung can include all forms of proliferative disorders affecting lung cells. Proliferative disorders of the lung can include lung cancer, precancerous conditions of the lung. Proliferative disorders of the lung can include hyperplasia, metaplasia, and dysplasia of the lung. Proliferative disorders of the lung can include asbestos-induced hyperplasia, squamous metaplasia, and benign reactive mesothelial metaplasia. Proliferative disorders of the lung can include replacement of columnar epithelium with stratified squamous epithelium, and mucosal dysplasia. Individuals exposed to inhaled injurious environmental agents such as cigarette smoke and asbestos may be at increased risk for developing proliferative disorders of the lung. Prior lung diseases that may predispose individuals to development of proliferative disorders of the lung can include chronic interstitial lung disease, necrotizing pulmonary disease, scleroderma, rheumatoid disease, sarcoidosis, interstitial pneumonitis, tuberculosis, repeated pneumonias, idiopathic pulmonary fibrosis, granulomata, asbestosis, fibrosing alveolitis, and Hodgkin's disease.

A “proliferative disorder of the colon” is a proliferative disorder involving cells of the colon. In one embodiment, the proliferative disorder of the colon is colon cancer. In one embodiment, compositions of the present application may be used to treat colon cancer or proliferative disorders of the colon. Colon cancer can include all forms of cancer of the colon. Colon cancer can include sporadic and hereditary colon cancers. Colon cancer can include malignant colon neoplasms, carcinoma in situ, typical carcinoid tumors, and atypical carcinoid tumors. Colon cancer can include adenocarcinoma, squamous cell carcinoma, and adenosquamous cell carcinoma. Colon cancer can be associated with a hereditary syndrome selected from the group consisting of hereditary nonpolyposis colorectal cancer, familial adenomatous polyposis, Gardner's syndrome, Peutz-Jeghers syndrome, Turcot's syndrome and juvenile polyposis. Colon cancer can be caused by a hereditary syndrome selected from the group consisting of hereditary nonpolyposis colorectal cancer, familial adenomatous polyposis, Gardner's syndrome, Peutz-Jeghers syndrome, Turcot's syndrome and juvenile polyposis.

Proliferative disorders of the colon can include all forms of proliferative disorders affecting colon cells. Proliferative disorders of the colon can include colon cancer, precancerous conditions of the colon, adenomatous polyps of the colon and metachronous lesions of the colon. A proliferative disorder of the colon can include adenoma. Proliferative disorders of the colon can be characterized by hyperplasia, metaplasia, and dysplasia of the colon. Prior colon diseases that may predispose individuals to development of proliferative disorders of the colon can include prior colon cancer. Current disease that may predispose individuals to development of proliferative disorders of the colon can include Crohn's disease and ulcerative colitis. A proliferative disorder of the colon can be associated with a mutation in a gene selected from the group consisting of p53, ras, FAP and DCC. An individual can have an elevated risk of developing a proliferative disorder of the colon due to the presence of a mutation in a gene selected from the group consisting of p53, ras, FAP and DCC.

A “proliferative disorder of the pancreas” is a proliferative disorder involving cells of the pancreas. Proliferative disorders of the pancreas can include all forms of proliferative disorders affecting pancreatic cells. Proliferative disorders of the pancreas can include pancreas cancer, a precancer or precancerous condition of the pancreas, hyperplasia of the pancreas, and dysplasia of the pancreas, benign growths or lesions of the pancreas, and malignant growths or lesions of the pancreas, and metastatic lesions in tissue and organs in the body other than the pancreas. Pancreatic cancer includes all forms of cancer of the pancreas. Pancreatic cancer can include ductal adenocarcinoma, adenosquamous carcinoma, pleomorphic giant cell carcinoma, mucinous adenocarcinoma, osteoclast-like giant cell carcinoma, mucinous cystadenocarcinoma, acinar carcinoma, unclassified large cell carcinoma, small cell carcinoma, pancreatoblastoma, papillary neoplasm, mucinous cystadenoma, papillary cystic neoplasm, and serous cystadenoma. Pancreatic cancer can also include pancreatic neoplasms having histologic and ultrastructural heterogeneity (e.g., mixed cell types).

A “proliferative disorder of the prostate” is a proliferative disorder involving cells of the prostate. Proliferative disorders of the prostate can include all forms of proliferative disorders affecting prostate cells. Proliferative disorders of the prostate can include prostate cancer, a precancer or precancerous condition of the prostate, benign growths or lesions of the prostate, and malignant growths or lesions of the prostate, and metastatic lesions in tissue and organs in the body other than the prostate. Proliferative disorders of the prostate can include hyperplasia, metaplasia, and dysplasia of the prostate.

A “proliferative disorder of the skin” is a proliferative disorder involving cells of the skin. Proliferative disorders of the skin can include all forms of proliferative disorders affecting skin cells. Proliferative disorders of the skin can include a precancer or precancerous condition of the skin, benign growths or lesions of the skin, melanoma, malignant melanoma and other malignant growths or lesions of the skin, and metastatic lesions in tissue and organs in the body other than the skin. Proliferative disorders of the skin can include hyperplasia, metaplasia, and dysplasia of the skin.

A “proliferative disorder of the ovary” is a proliferative disorder involving cells of the ovary. Proliferative disorders of the ovary can include all forms of proliferative disorders affecting cells of the ovary. Proliferative disorders of the ovary can include a precancer or precancerous condition of the ovary, benign growths or lesions of the ovary, ovarian cancer, malignant growths or lesions of the ovary, and metastatic lesions in tissue and organs in the body other than the ovary. Proliferative disorders of the skin can include hyperplasia, metaplasia, and dysplasia of cells of the ovary.

A “proliferative disorder of the breast” is a proliferative disorder involving cells of the breast. Proliferative disorders of the breast can include all forms of proliferative disorders affecting breast cells. Proliferative disorders of the breast can include breast cancer, a precancer or precancerous condition of the breast, benign growths or lesions of the breast, and malignant growths or lesions of the breast, and metastatic lesions in tissue and organs in the body other than the breast. Proliferative disorders of the breast can include hyperplasia, metaplasia, and dysplasia of the breast.

A cancer that is to be treated can be staged according to the American Joint Committee on Cancer (AJCC) TNM classification system, where the tumor (T) has been assigned a stage of TX, T1, T1mic, T1a, T1b, T1c, T2, T3, T4, T4a, T4b, T4c, or T4d; and where the regional lymph nodes (N) have been assigned a stage of NX, N0, N1, N2, N2a, N2b, N3, N3a, N3b, or N3c; and where distant metastasis (M) can be assigned a stage of MX, M0, or M1. A cancer that is to be treated can be staged according to an American Joint Committee on Cancer (AJCC) classification as Stage I, Stage IIA, Stage IIB, Stage IIIA, Stage IIIB, Stage IIIC, or Stage IV. A cancer that is to be treated can be assigned a grade according to an AJCC classification as Grade GX (e.g., grade cannot be assessed), Grade 1, Grade 2, Grade 3 or Grade 4. A cancer that is to be treated can be staged according to an AJCC pathologic classification (pN) of pNX, pN0, PN0 (1−), PN0 (1+), PN0 (mol−), PN0 (mol+), PNT, PNT(mi), PNTa, PNTb, PNTc, pN2, pN2a, pN2b, pN3, pN3a, pN3b, or pN3c.

A cancer that is to be treated can include a tumor that has been determined to be less than or equal to about 2 centimeters in diameter. A cancer that is to be treated can include a tumor that has been determined to be from about 2 to about 5 centimeters in diameter. A cancer that is to be treated can include a tumor that has been determined to be greater than or equal to about 3 centimeters in diameter. A cancer that is to be treated can include a tumor that has been determined to be greater than 5 centimeters in diameter. A cancer that is to be treated can be classified by microscopic appearance as well differentiated, moderately differentiated, poorly differentiated, or undifferentiated. A cancer that is to be treated can be classified by microscopic appearance with respect to mitosis count (e.g., amount of cell division) or nuclear pleiomorphism (e.g., change in cells). A cancer that is to be treated can be classified by microscopic appearance as being associated with areas of necrosis (e.g., areas of dying or degenerating cells). A cancer that is to be treated can be classified as having an abnormal karyotype, having an abnormal number of chromosomes, or having one or more chromosomes that are abnormal in appearance. A cancer that is to be treated can be classified as being aneuploid, triploid, tetraploid, or as having an altered ploidy. A cancer that is to be treated can be classified as having a chromosomal translocation, or a deletion or duplication of an entire chromosome, or a region of deletion, duplication or amplification of a portion of a chromosome.

A cancer that is to be treated can be evaluated by DNA cytometry, flow cytometry, or image cytometry. A cancer that is to be treated can be typed as having 10%, 20%, 30%, 40%, 50%, 60%, 70%, 80%, or 90% of cells in the synthesis stage of cell division (e.g., in S phase of cell division). A cancer that is to be treated can be typed as having a low S-phase fraction or a high S-phase fraction.

As used herein, a “normal cell” is a cell that cannot be classified as part of a “proliferative disorder”. A normal cell lacks unregulated or abnormal growth, or both, that can lead to the development of an unwanted condition or disease. In one embodiment, a normal cell possesses normally functioning cell cycle checkpoint control mechanisms.

One skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3rd edition), Cold Spring Harbor Press, Cold Spring Harbor, N.Y. (2000); Coligan et al., Current Protocols in Immunology, John Wiley & Sons, N.Y.; Erma et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, Pa., 18th edition (1990). These texts can, of course, also be referred to in making or using an aspect of the application.

In certain embodiments, compounds of the application are useful in the treatment of proliferative disorders (e.g., cancer, benign neoplasms, inflammatory disease, and autoimmune diseases). In certain embodiments, according to the methods of treatment of the present application, levels of cell proteins of interest, e.g., pathogenic and oncogenic proteins are modulated, or their growth is inhibited by contacting said cells with an compound or composition, as described herein. In other embodiments, the compounds are useful in treating cancer.

In certain embodiments, the method involves the administration of a therapeutically effective amount of the compound or a pharmaceutically acceptable derivative thereof to a subject (including, but not limited to a human or animal) in need of it. In certain embodiments, the compounds are useful for the treatment of cancer (including, but not limited to, glioblastoma, retinoblastoma, breast cancer, cervical cancer, colon and rectal cancer, leukemia, lymphoma, lung cancer (including, but not limited to small cell lung cancer), melanoma and/or skin cancer, multiple myeloma, non-Hodgkin's lymphoma, ovarian cancer, pancreatic cancer, prostate cancer and gastric cancer, bladder cancer, uterine cancer, kidney cancer, testicular cancer, stomach cancer, brain cancer, liver cancer, or esophageal cancer).

In certain embodiments, the anticancer agents are useful in the treatment of cancers and other proliferative disorders, including, but not limited to breast cancer, cervical cancer, colon and rectal cancer, leukemia, lung cancer, melanoma, multiple myeloma, non-Hodgkin's lymphoma, ovarian cancer, pancreatic cancer, prostate cancer, and gastric cancer. In certain embodiments, the anticancer agents are active against solid tumors.

Additionally, the present application provides pharmaceutically acceptable derivatives of the compounds, and methods of treating a subject using these compounds, pharmaceutical compositions thereof, or either of these in combination with one or more additional therapeutic agents.

For example, other therapies or anticancer agents that may be used in combination with the compounds disclosed herein including surgery, radiotherapy, endocrine therapy, biologic response modifiers (interferons, interleukins, and tumor necrosis factor (TNF), to name a few), hyperthermia and cryotherapy, agents to attenuate any adverse effects (e.g., antiemetics), and other approved chemotherapeutic drugs, including, but not limited to, alkylating drugs (mechlorethamine, chlorambucil, cyclophosphamide, melphalan, ifosfamide), antimetabolites (methotrexate), purine antagonists and pyrimidine antagonists (6-mercaptopurine, 5-fluorouracil, cytarabine, gemcitabine), spindle poisons (vinblastine, vincristine, vinorelbine, paclitaxel), podophyllotoxins (etoposide, irinotecan, topotecan), antibiotics (doxorubicin, bleomycin, mitomycin), nitrosoureas (carmustine, lomustine), inorganic ions (cisplatin, carboplatin), enzymes (asparaginase), and hormones (tamoxifen, leuprolide, flutamide, and megestrol), to name a few. For a more comprehensive discussion of overview of cancer therapy see The Merck Manual, Twentieth Ed. 2020, the entire contents of which are hereby incorporated by reference. See also the National Cancer Institute (NCI) website (www.nci.nih.gov) and the Food and Drug Administration (FDA) website for a list of the FDA approved oncology drugs (www.fda.gov/cder/cancer/druglistframe).

In certain embodiments, the pharmaceutical compositions comprising the compounds disclosed herein further comprise one or more additional therapeutically active ingredients (e.g., chemotherapeutic and/or palliative). For purposes of the application, the term “palliative” refers to treatment that is focused on the relief of symptoms of a disease and/or side effects of a therapeutic regimen, but is not curative. For example, palliative treatment encompasses painkillers, antinausea medications and anti-sickness drugs. In addition, chemotherapy, radiotherapy and surgery can all be used palliatively (that is, to reduce symptoms without going for cure; e.g., for shrinking tumors and reducing pressure, bleeding, pain and other symptoms of cancer).

### Administration, Pharmaceutical Compositions

Administration of the disclosed compounds and pharmaceutical compositions can be accomplished via any mode of administration for therapeutic agents. These modes include systemic or local administration such as oral, nasal, parenteral, transdermal, subcutaneous, vaginal, buccal, rectal or topical administration modes.

Depending on the intended mode of administration, the disclosed compositions can be in solid, semi-solid or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices. Likewise, they can also be administered in intravenous (both bolus and infusion), intraperitoneal, subcutaneous or intramuscular form, and all using forms well known to those skilled in the pharmaceutical arts.

Illustrative pharmaceutical compositions are tablets and gelatin capsules comprising a compound of the disclosure and a pharmaceutically acceptable carrier, such as a) a diluent, e.g., purified water, triglyceride oils, such as hydrogenated or partially hydrogenated vegetable oil, or mixtures thereof, com oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride, and/or polyethylene glycol; for tablets also; c) a binder, e.g., magnesium aluminum silicate, starch paste, gelatin, tragacanth, methylcellulose, sodium carboxymethyleellulose, magnesium carbonate, natural sugars such as glucose or beta-lactose, corn sweeteners, natural and synthetic gums such as acacia, tragacanth or sodium alginate, waxes, and/or polyvinylpyrrolidone, if desired; d) a disintegrant, e.g., starches, agar, methyl cellulose, bentonite, xanthan gum, algic acid or its sodium salt, or effervescent mixtures; e) absorbent, colorant, flavorant and sweetener; f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the compound such as cyclodextrin, hydroxypropyl-cyclodextrin, PEG400, PEG200.

Liquid, particularly injectable, compositions can, for example, be prepared by dissolution, dispersion, etc. For example, the disclosed compound is dissolved in or mixed with a pharmaceutically acceptable solvent such as, for example, water, saline, aqueous dextrose, glycerol, ethanol, and the like, to thereby form an injectable isotonic solution or suspension. Proteins such as albumin, chylomicron particles, or serum proteins can be used to solubilize the disclosed compounds.

The disclosed compounds can be also formulated as a suppository that can be prepared from fatty emulsions or suspensions; using polyalkylene glycols such as propylene glycol, as the carrier.

The disclosed compounds can also be administered in the form of liposome delivery systems, such as small unilamellar vesicles, large unilamellar vesicles, and multilamellar vesicles. Liposomes can be formed from a variety of phospholipids, containing cholesterol, stearylamine or phosphatidylcholines.

In some embodiments, a film of lipid components is hydrated with an aqueous solution of drug to a form lipid layer encapsulating the drug, as described in U.S. Pat. No. 5,262,564, which is hereby incorporated by reference in its entirety.

Disclosed compounds can also be delivered by the use of monoclonal antibodies as individual carriers to which the disclosed compounds are coupled. The disclosed compounds can also be coupled with soluble polymers as targetable drug carriers. Such polymers can include polyvinylpyrrolidone, pyran copolymer, polyhydroxypropylmethacrylamide-phenol, polyhydroxyethylaspanamidephenol, or polyethyleneoxidepolylysine substituted with palmitoyl residues. Furthermore, the disclosed compounds can be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates, and cross-linked or amphipathic block copolymers of hydrogels. In one embodiment, disclosed compounds are not covalently bound to a polymer, e.g., a polycarboxylic acid polymer, or a polyacrylate.

Parental injectable administration is generally used for subcutaneous, intramuscular or intravenous injections and infusions. Injectables can be prepared in conventional forms, either as liquid solutions or suspensions or solid forms suitable for dissolving in liquid prior to injection.

Another aspect of the disclosure is directed to pharmaceutical compositions comprising a compound of Formula 1, and a pharmaceutically acceptable carrier. The pharmaceutically acceptable carrier may further include an excipient, diluent, or surfactant.

Compositions can be prepared according to conventional mixing, granulating or coating methods, respectively, and the present pharmaceutical compositions can contain from about 0.1% to about 99%, from about 5% to about 90%, or from about 1% to about 20% of the disclosed compound by weight or volume.

In one embodiment, the disclosure provides a kit comprising two or more separate pharmaceutical compositions, at least one of which contains a compound of the present disclosure. In one embodiment, the kit comprises means for separately retaining said compositions, such as a container, divided bottle, or divided foil packet. An example of such a kit is a blister pack, as typically used for the packaging of tablets, capsules and the like.

The kit of the disclosure may be used for administering different dosage forms, for example, oral and parenteral, for administering the separate compositions at different dosage intervals, or for titrating the separate compositions against one another. To assist compliance, the kit of the disclosure typically comprises directions for administration.

Pharmaceutical dosage forms of a compound of this disclosure may be manufactured by any of the methods well-known in the art, such as, for example, by conventional mixing, sieving, dissolving, melting, granulating, dragee-making, tableting, suspending, extruding, spray-drying, levigating, emulsifying, (nano-/micro-) encapsulating, entrapping, or lyophilization processes. As noted above, the compositions of this disclosure can include one or more physiologically acceptable inactive ingredients that facilitate processing of active molecules into preparations for pharmaceutical use.

As noted above, the compositions are comprised of, in general, a compound of this disclosure in combination with at least one pharmaceutically acceptable excipient. Acceptable excipients are non-toxic, aid administration, and do not adversely affect the therapeutic benefit of the claimed compounds. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semi-solid excipients may be selected from glycerol, propylene glycol, water, ethanol and various oils, including those of petroleum, animal, vegetable or synthetic origin, e.g., peanut oil, soybean oil, mineral oil, sesame oil, etc. In some embodiments, liquid carriers, particularly for injectable solutions, include water, saline, aqueous dextrose, and glycols.

Compressed gases may be used to disperse a compound of this disclosure in an aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990).

The compositions of this disclosure may, if desired, be presented in a pack or dispenser device containing one or more unit dosage forms containing the active ingredient. Such a pack or device may, for example, comprise metal or plastic foil, such as a blister pack, or glass, and rubber stoppers such as in vials. The pack or dispenser device may be accompanied by instructions for administration. Compositions comprising a compound of this disclosure that can be formulated in a compatible pharmaceutical carrier may also be prepared, placed in an appropriate container, and labeled for treatment of an indicated condition.

The amount of the compound in a formulation can vary within the full range employed by those skilled in the art. Typically, the formulation will contain, on a weight percent (wt %) basis, from about 0.01-99.99 wt % of a compound of this disclosure based on the total formulation, with the balance being one or more suitable pharmaceutical excipients. In one embodiment, the compound is present at a level of about 1-80 wt %. Representative pharmaceutical formulations are described below.

### Formulation Examples

The following are representative pharmaceutical formulations containing a compound of this disclosure.

### Formulation Example 1—Tablet formulation

The following ingredients are mixed intimately and pressed into single scored tablets.

| | Quantity per |
|---|---|
| Ingredient | tablet, mg |
| compound of this disclosure | 400 |
| Cornstarch | 50 |
| croscarmellose sodium | 25 |
| Lactose | 120 |
| magnesium stearate | 5 |

### Formulation Example 2—Capsule Formulation

The following ingredients are mixed intimately and loaded into a hard-shell gelatin capsule.

| | Quantity per |
|---|---|
| Ingredient | capsule, mg |
| compound of this disclosure | 200 |
| lactose, spray-dried | 148 |
| magnesium stearate | 2 |

### Formulation Example 3—Suspension Formulation

The following ingredients are mixed to form a suspension for oral administration.

| Ingredient | Amount | |
|---|---|---|
| compound of this disclosure | 1.0 | g |
| fumaric acid | 0.5 | g |
| sodium chloride | 2.0 | g |
| methyl paraben | 0.15 | g |
| propyl paraben | 0.05 | g |
| granulated sugar | 25.0 | g |
| sorbitol (70% solution) | 13.00 | g |
| Veegum K (Vanderbilt Co.) | 1.0 | g |
| Flavoring | 0.035 | mL |
| Colorings | 0.5 | mg |

| | |
|---|---|
| distilled water | q.s. to 100 mL |

### Formulation Example 4—Injectable Formulation

The following ingredients are mixed to form an injectable formulation.

| Ingredient | Amount |
|---|---|
| compound of this disclosure | 0.2 mg-20 mg |
| sodium acetate buffer solution, 0.4M | 2.0 mL |
| HC1 (1N) or NaOH (1N) | q.s. to suitable pH |
| water (distilled, sterile) | q.s. to 20 mL |

### Formulation Example 5—Suppository Formulation

A suppository of total weight 2.5 g is prepared by mixing the compound of this disclosure with Witepsol® H-15 (triglycerides of saturated vegetable fatty acid; Riches-Nelson, Inc., New York), and has the following composition:

| Ingredient | Amount |
|---|---|
| Compound of this disclosure | 500 mg |
| Witepsol ® H-15 | balance |

### Dosing

The dosage regimen utilizing the disclosed compound is selected in accordance with a variety of factors including type, species, age, weight, sex, and medical condition of the patient; the severity of the condition to be treated; the route of administration; the renal or hepatic function of the patient; and the particular disclosed compound employed. A physician or veterinarian of ordinary skill in the art can readily determine and prescribe the effective amount of the drug required to prevent, counter or arrest the progress of the condition.

Effective dosage amounts of the disclosed compounds, when used for the indicated effects, range from about 0.5 mg to about 5000 mg of the disclosed compound as needed to treat the condition. Compositions for in vivo or in vitro use can contain about 0.5, 5, 20, 50, 75, 100, 150, 250, 500, 750, 1000, 1250, 2500, 3500, or 5000 mg of the disclosed compound, or, in a range of from one amount to another amount in the list of doses. In one embodiment, the compositions are in the form of a tablet that can be scored.

### EXAMPLES

This disclosure is further understood by reference to the following examples, which are intended to be purely exemplary of this disclosure. This disclosure is not limited in scope by the exemplified embodiments, which are intended as illustrations of single aspects of this disclosure only. Any methods that are functionally equivalent are within the scope of this disclosure. Various modifications of this disclosure in addition to those described herein will become apparent to those skilled in the art from the foregoing description and accompanying FIGURES. Such modifications fall within the scope of the appended claims.

In the specification and in the examples below, all temperatures are in degrees Celsius. In addition, the following abbreviations have the following meanings. If not defined, these abbreviations have their art recognized meaning.

| Abbreviation | Meaning |
|---|---|
| δ | chemical shift (ppm) |
| Å | Angstrom |
| ACN or MeCN | acetonitrile |
| Boc or t-Boc | tert-butoxycarbonyl |
| Cbz | benzyloxycarbonyl |
| CDCl₃ | deuterated chloroform |
| DC₅₀ | concentration that resulted in a 50% targeted protein degradation |
| DCC | N,N-dicyclohexylcarbodiimide |
| DCM | dichloromethane |
| DHA | docosahexaenoic acid |
| DIEA | diisopropylethylamine |
| DMAP | 4-dimethylaminopyridine |
| DMF | N,N-dimethylformamide |
| DMSO | dimethylsulfoxide |
| d₆-DMSO | deuterated dimethylsulfoxide |
| d₄-MeOD | deuterated methanol |
| EPA | eicosapentaenoic acid |
| eq. | equivalent(s) |
| ESI | electrospray ionization |
| EtOH | ethanol |
| Fmoc | fluorcnylmcthyloxycarbonyl |
| g | grams |
| ¹H NMR | proton nuclear magnetic resonance spectroscopy |
| h or hr | hour(s) |
| HATU | 1-[bis(dimethylamino)methylene]-1H-1,2,3-triazolo[4,5-b]pyridinium 3- |
| | oxide hexafluorophosphate |
| HPLC | high performance liquid chromatography |
| HPMC | hydroxypropyl methylcellulose |
| L | liter |
| LC | liquid chromatography |
| LC-MS | liquid chromatography - mass spectrometry |
| LiHMDS | lithium bis(trimethylsilyl)amide |
| M | molar |
| mCPBA | meta-Chloroperoxybenzoic acid |
| mg | milligrams |
| m/z | mass-to-charge ratio |
| min | minute(s) |
| mmol | millimole |
| mL | milliliter |
| MTBE | methyl tert-butyl ether |
| μL | microliter |
| μmol or μmol | micromole |
| μM | micromolar |
| N | normal |
| Pd(dppf)Cl₂ | 1,1′-bis(diphenylphosphino)ferrocene]dichloropalladium(II) |
| Pd(OAc)₂ | palladium(II) acetate |
| Pd(PPh₃)₂Cl₂ | bis(triphenylphosphine)palladium(II) dichloride |
| PPh₃ | triphenylphosphine |
| q.s. | amount which is sufficient |
| TEA | triethylamine |
| TFA | trifluoroacetic acid |
| THF | tetrahydrofuran |
| UV | ultraviolet |
| wt % | weight percent |
| NMR abbreviations | br = broad |
| | d = doublet |
| | dd = doublet of doublets |
| | m = multiplet |
| | q = quartet |
| | s = singlet |
| | t = triplet |

### LC-MS Methods (General Method)

Method A: Experiments were performed using a Luna® 5 μm C₁₈ (2) 100 Å, LC Column 250 x 21.2 mm, AXIA™ Packed (00G-4252-P0-AX), at a flow rate of 20 mL/min, and a mass spectrometer using ESI as ionization source. The solvent A was 4.0 mL of TFA in 4 L of water, and solvent B was 4.0 mL of TFA in 4 L of acetonitrile. The gradient consisted of 10-100% solvent B over 20 minutes, LC column temperature was 40° C. UV absorbance was collected at 220 nm and 254 nm.

### Example 1

### Preparation of N-(3-(4-(4-(3-amino-6-(2-hydroxyphenyl)pyridazin-4-yl)piperazin-1-yl)pyrimidin-2-yl)prop-2-yn-1-yl)-1,5-dimethyl-1H-pyrazole-3-carboxamide (Compound P-12)

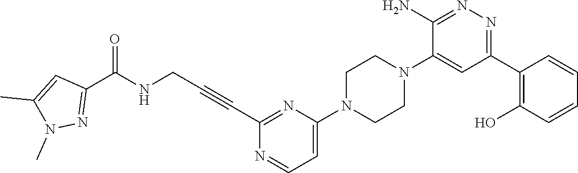 [SMILES: Cc1cc(C(=O)NCC#Cc2nccc(N3CCN(c4cc(-c5ccccc5O)nnc4N)CC3)n2)nn1C]

### Step 1:

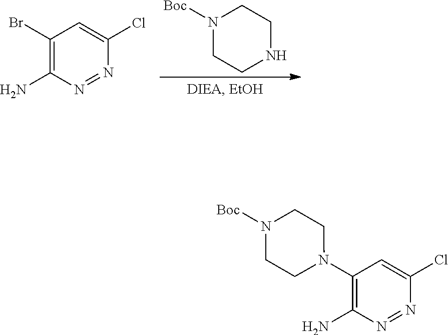 [SMILES: Nc1nnc(Cl)cc1Br ; CC(C)(C)OC(=O)N1CCNCC1 ; CC(C)(C)OC(=O)N1CCN(c2cc(Cl)nnc2N)CC1]

To a solution of 4-bromo-6-chloropyridazin-3-amine (1 g, 4.80 mmol, 1 eq) in EtOH (10 mL) was added DIEA (1.86 g, 14.39 mmol, 2.51 mL, 3 eq) and tert-butyl piperazine-1-carboxylate (1.34 g, 7.20 mmol, 1.5 eq). The mixture was stirred at 80° C. for 12 hr. The reaction was concentrated under reduced pressure to give a residue. The residue was dissolved in ethyl acetate (20 mL) and washed with water, dried over magnesium sulfate, and the solvents evaporated in vacuo to give tert-butyl 4-(3-amino-6-chloropyridazin-4-yl)piperazine-1-carboxylate.

### Step 2:

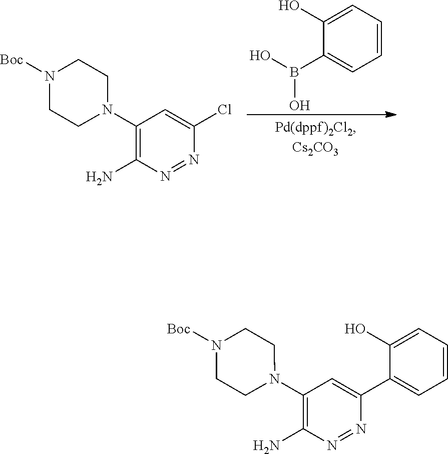 [SMILES: CC(C)(C)OC(=O)N1CCN(c2cc(Cl)nnc2N)CC1 ; OB(O)c1ccccc1O ; CC(C)(C)OC(=O)N1CCN(c2cc(-c3ccccc3O)nnc2N)CC1]

A mixture of tert-butyl 4-(3-amino-6-chloropyridazin-4-yl)piperazine-1-carboxylate (1.3 g, 4.14 mmol, 1 eq), (2-hydroxyphenyl)boronic acid (857.16 mg, 6.21 mmol, 1.5 eq), Pd(dppf)₂Cl₂ (303.15 mg, 414.30 μmol, 0.1eq), and Cs₂CO₃ (8.10 g, 24.86 mmol, 6 eq) in dioxane (14 mL) and H₂O (1.4 mL) was degassed and purged with N₂. The mixture was stirred at 80° C. for 12 hours under N₂ atmosphere. The reaction was extracted with ethyl acetate (5 mL), and the organic phase was concentrated under reduced pressure to give a residue. The residue was purified by column chromatography (SiO₂, 0 to 100% ethyl acetate in petroleum ether) to give tert-butyl 4-(3-amino-6-(2-hydroxyphenyl)pyridazin-4-yl)piperazine-1-carboxylate. ¹H NMR (400 MHz, d₆-DMSO) δ 7.92 (dd, J=8.25, 1.25 Hz, 1H) 7.55 (s, 1H) 7.18-7.29 (m, 1H) 6.82-6.95 (m, 2H) 6.38 (s, 2H) 3.55 (br s, 5H) 2.99-3.08 (m, 4H) 1.43 (s, 9H).

### Step 3:

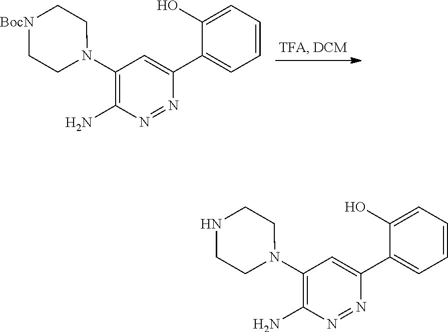 [SMILES: CC(C)(C)OC(=O)N1CCN(c2cc(-c3ccccc3O)nnc2N)CC1 ; Nc1nnc(-c2ccccc2O)cc1N1CCNCC1]

To a solution of tert-butyl 4-(3-amino-6-(2-hydroxyphenyl)pyridazin-4-yl)piperazine-1-carboxylate (1 g, 2.69 mmol, 1 eq) in DCM (8 mL), was added TFA (4 mL). The mixture was stirred at 25° C. for 12 hr. The reaction was concentrated under reduced pressure to give a residue, which was triturated with MTBE to give 2-(6-amino-5-(piperazin-1-yl)pyridazin-3-yl)phenol as a TFA salt. ¹H NMR (400 MHz, d₆-DMSO) δ 9.34-9.61 (m, 2H) 8.27-8.48 (m, 1H) 7.79 (br d, J=7.50 Hz, 1H) 7.61 (s, 1H) 7.31 (hr t, J=7.50 Hz, 1H) 7.05-7.22 (m, 2H) 6.99 (hr d, J=8.13 Hz, 1H) 6.93 (hr t, J=7.44 Hz, 1H) 3.37 (br s, 7H).

### Step 4:

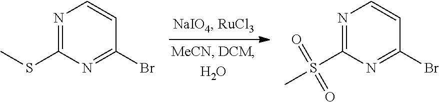

To a solution of 4-bromo-2-(methylthio)pyrimidine (5 g, 24.38 mmol, 1 eq) in MeCN (20 mL), DCM (20 mL), and H₂O (20 mL) at 0° C., was added NaIO₄ (10.41 g, 48.76 mmol, 2 eq). RuCl₃ (0.1 g, 0.02 eq) was added and the reaction warmed to 25° C. and stirred for 12 hours. The reaction mixture was diluted with water (50 mL) and extracted with DCM (100 mL). The organic layer was separated and washed with saturated Na₂S₂O₃ (50 mL) and dried over sodium sulfate. The organic layer was concentrated to give a residue, which was purified by column chromatography to give 4-bromo-2-(methylsulfonyl)pyrimidine.

### Step 5:

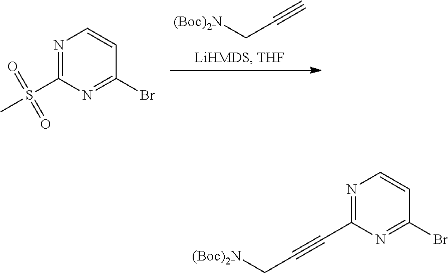

Di-tert-butyl prop-2-yn-1-yliminodicarbonate (2.16 g, 8.44 mmol, 1 eq) was added to a solution of LiHMDS (10 mL, 1.2 eq, 1N) in THF (30 mL) at −78° C., and stirred for 1 h. 4-Bromo-2-(methylsulfonyl)pyrimidine (2 g, 8.44 mmol, 1 eq) in DCM (10 mL) was added dropwise and stirred at −78° C. for 2 h. The reaction mixture was quenched with saturated NH₄Cl (30 mL), diluted with water (50 mL), and extracted with MTBE (50 mL). The organic layer was separated, dried over sodium sulfate, and concentrated to give a residue. The residue was purified by column chromatography to give di-tert-butyl (3-(4-bromopyrimidin-2-yl)prop-2-yn-1-yl)iminodicarbonate. ¹H NMR (400 MHz, CDCl₃) δ 8.44 (d, J=6.4 Hz, 1H), 7.45 (d, J=6.4 Hz, 1H), 4.66 (s, 2H), 1.54 (s, 18H).

### Step 6:

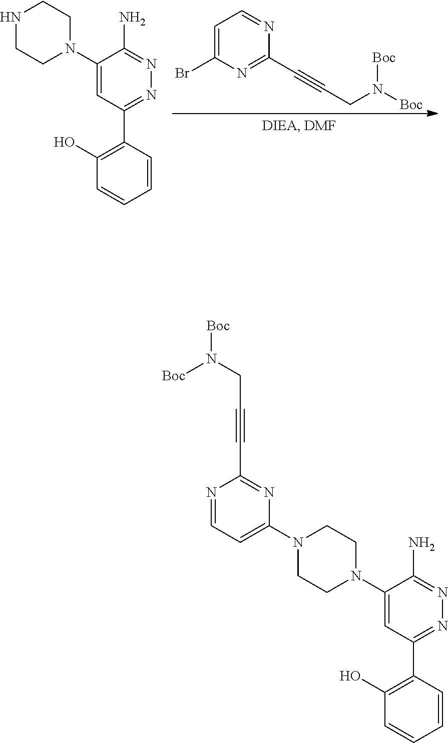

A solution of 2-(6-amino-5-(piperazin-1-yl)pyridazin-3-yl)phenol (802 mg, 2.08 mmol, 1 eq), di-tert-butyl (3-(4-bromopyrimidin-2-yl)prop-2-yn-1-yl)iminodicarbonate (650 mg, 2.08 mmol, 1 eq), and DIEA (807 mg, 6.25 mmol, 3 eq) in DMF (5.0 mL) was stirred at room temperature overnight. 1M citric acid solution was added to the reaction mixture until pH=~4. The precipitate was filtered and washed with ethyl acetate and dried to give tert-butyl (3-(4-(4-(3-amino-6-(2-hydroxyphenyl)pyridazin-4-yl)piperazin-1-yl)pyrimidin-2-yl)prop-2-yn-1-yl)(tert-butoxycarbonyl)carbamate.

### Step 7:

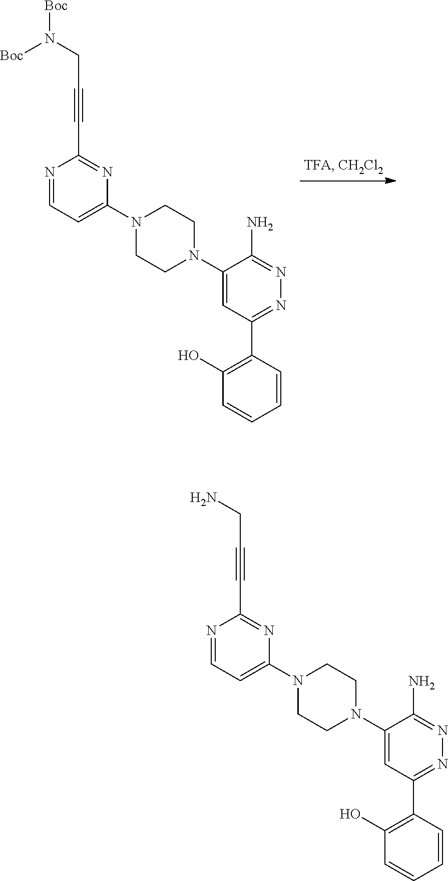 [SMILES: CC(C)(C)OC(=O)N(CC#Cc1nccc(N2CCN(c3cc(-c4ccccc4O)nnc3N)CC2)n1)C(=O)OC(C)(C)C ; NCC#Cc1nccc(N2CCN(c3cc(-c4ccccc4O)nnc3N)CC2)n1]

To a solution of tert-butyl (3-(4-(4-(3-amino-6-(2-hydroxyphenyl)pyridazin-4-yl)piperazin-1-yl)pyrimidin-2-yl)prop-2-yn-1-yl)(tert-butoxycarbonyl)carbamate (162 mg, 322 μmol) in dichloromethane (3 mL), was added trifluoroacetic acid (2 mL). The solution was stirred at room temperature for 1 hour. The solvents were evaporated in vacuo to give 2-(6-amino-5-(4-(2-(3-aminoprop-1-yn-1-yl)pyrimidin-4-yl)piperazin-1-yl)pyridazin-3-yl)phenol.

### Step 8:

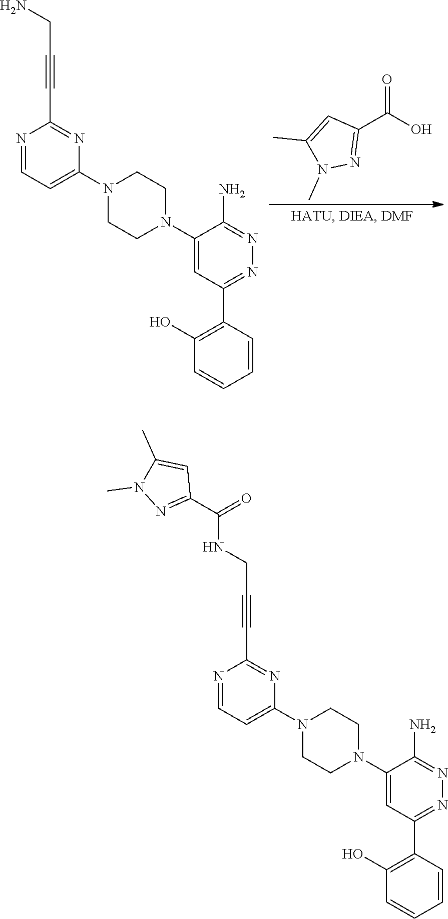

A solution of 1,5-dimethyl-1H-pyrazole-3-carboxylic acid (3.3 mg, 23.8 μmol) and HATU (9.1 mg, 24 μmol) in DMF (0.3 mL) was stirred at room temperature for 2 minutes. 2-(6-amino-5-(4-(2-(3-aminoprop-1-yn-1-yl)pyrimidin-4-yl)piperazin-1-yl)pyridazin-3-yl)phenol (15 mg, 24 μmol) and DIEA (12.3 mg, 95.2 μmol. 4 eq) in DMF (0.2 mL) was added. The solution was stirred at room temperature for 5 hours. The solution was purified by prep-HPLC (column: Phenomenex Luna C₁₈ 100×40 mm×5 mm; mobile phase: (0.1% formic acid in water-0.1% formic acid in ACN); B %: 5%-50%, 8 min). The fractions were combined and lyophilized to give N-(3-(4-(4-(3-amino-6-(2-hydroxyphenyl)pyridazin-4-yl)piperazin-1-yl)pyrimidin-2-yl)prop-2-yn-1-yl)-1,5-dimethyl-1H-pyrazole-3-carboxamide.

### Example 2

### Preparation of N-(3-(4-(3-(3-amino-6-(2-hydroxyphenyl)pyridazin-4-yl)-3,8-diazabicyclo[3.2.1]octan-8-yl)pyrimidin-2-yl)prop-2-yn-1-yl)pyrazolo[1,5-a]pyrimidine-2-carboxamide (Compound P-119)

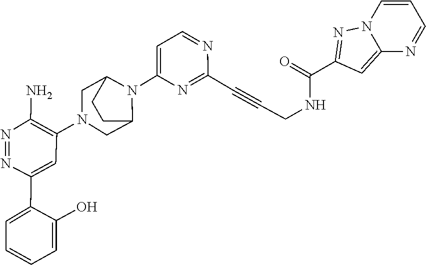 [SMILES: Nc1nnc(-c2ccccc2O)cc1N1CC2CCC(C1)N2c1ccnc(C#CCNC(=O)c2cc3ncccn3n2)n1]

### Step 1:

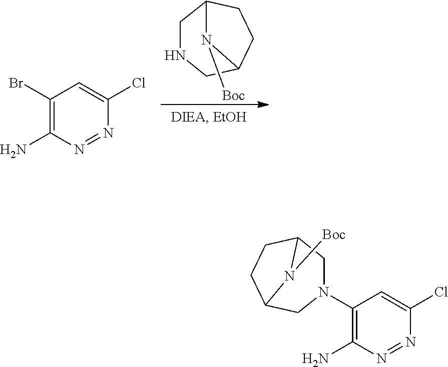 [SMILES: Nc1nnc(Cl)cc1Br ; CC(C)(C)OC(=O)N1C2CCC1CNC2 ; CC(C)(C)OC(=O)N1C2CCC1CN(c1cc(Cl)nnc1N)C2]

To a solution of 4-bromo-6-chloropyridazin-3-amine (1 g, 4.80 mmol, 1 eq.) in EtOH (10 mL), was added DIEA (1.86 g, 14.39 mmol, 2.51 mL, 3 eq.) and tert-butyl 3,8-diazabicyclo[3.2.1]octane-8-carboxylate (1.53 g, 7.20 mmol, 1.5 eq). The mixture was stirred at 100° C. for 12 hr. The reaction was concentrated under reduced pressure to give a residue. The residue was triturated with MTBE to give tert-butyl 3-(3-amino-6-chloropyridazin-4-yl)-3,8-diazabicyclo[3.2.1]octane-8-carboxylate, m/z (ESI⁺): 340.2 (M+H)⁺.

### Step 2:

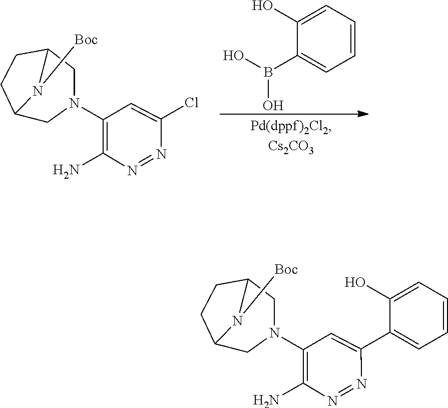 [SMILES: CC(C)(C)OC(=O)N1C2CCC1CN(c1cc(Cl)nnc1N)C2 ; OB(O)c1ccccc1O ; CC(C)(C)OC(=O)N1C2CCC1CN(c1cc(-c3ccccc3O)nnc1N)C2]

A mixture of tert-butyl 3-(3-amino-6-chloropyridazin-4-yl)-3,8-diazabicyclo[3.2.1]octane-8-carboxylate (2.4 g, 7.06 mmol, 1 eq), (2-hydroxyphenyl)boronic acid (1.46 g, 10.59 mmol, 1.5 eq), Pd(dppf)₂Cl₂ (516.78 mg, 706.26 μmol, 0.1 eq), and Cs₂CO₃ (13.81 g, 42.38 mmol, 6 eq) in dioxane (20 mL) and H₂O (2 mL) was degassed and purged with N₂. The mixture was stirred at 80° C. for 12 hr under N₂ atmosphere. The reaction was extracted with ethyl acetate (5 mL), and the organic phase was concentrated under reduced pressure to give a residue. The residue was purified by column chromatography (SiO₂, 0 to 100% ethyl acetate in petroleum ether) to give tert-butyl 3-(3-amino-6-(2-hydroxyphenyl)pyridazin-4-yl)-3,8-diazabicyclo[3.2.1]octane-8-carboxylate, m/z (ESI⁺) 298.2.2 (M−100+H)⁺.

### Step 3:

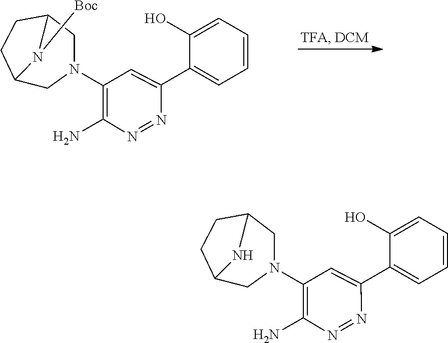 [SMILES: CC(C)(C)OC(=O)N1C2CCC1CN(c1cc(-c3ccccc3O)nnc1N)C2 ; Nc1nnc(-c2ccccc2O)cc1N1CC2CCC(C1)N2]

To a solution tert-butyl 3-(3-amino-6-(2-hydroxyphenyl)pyridazin-4-yl)-3,8-diazabicyclo[3.2.1]octane-8-carboxylate (0.92 g, 2.31 mmol, 1 eq) in DCM (6 mL), was added TFA (3 mL). The mixture was stirred at 25° C. for 12 hr. The reaction was concentrated under reduced pressure to give a residue, which was triturated with MTBE to give 2-(6-amino-5-(3,8-diazabicyclo[3.2.1]octan-3-yl)pyridazin-3-yl)phenol as a TFA salt. ¹H NMR (400 MHz, d₆-DMSO) δ 9.15 (br s, 2H) 7.69 (br d, J=7.25 Hz, 1H) 7.57 (s, 1H) 7.36 (br t, J=7.63 Hz, 1H) 6.76-7.09 (m, 4H) 4.18 (br s, 2H) 3.69 (br d, J=12.63 Hz, 2H) 3.25 (br d, J=13.13 Hz, 2H) 2.23 (br d, J=7.75 Hz, 2H) 1.90-2.02 (m, 2H).

### Step 4:

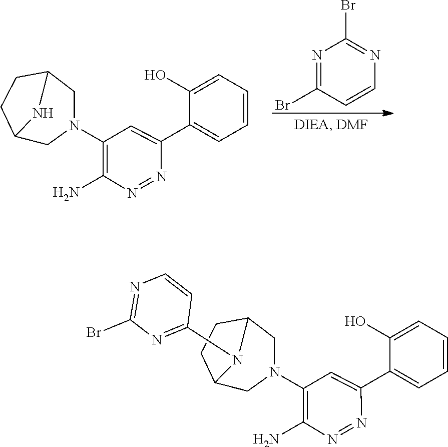

To a solution of 2-(6-amino-5-(3,8-diazabicyclo[3.2.1]octan-3-yl)pyridazin-3-yl)phenol trifluoroacetic acid (250 mg, 608 μmol, 1.0 eq) in DMF (4 mL), was added DIEA (259 mg, 2.01 mmol, 3.3 eq) and 2,4-dibromopyrimidine (145 mg, 608 μmol, 1 eq). The reaction was stirred at room temperature for 16 hours. The solution was purified by prep-HPLC (column: Phenomenex Luna C₁₈ 100 mm×40 mm×5 μm; mobile phase: (0.1% formic acid in water-0.1% formic acid in ACN); B %: 20%-80%, 8 min). The fractions were combined and lyophilized to give 2-(6-amino-5-(8-(2-bromopyrimidin-4-yl)-3,8-diazabicyclo[3.2.1]octan-3-yl)pyridazin-3-yl)phenol. ¹H NMR (400 MHz, d₆-DMSO) δ 8.02 (d, J=6.0 Hz, 1H), 7.93 (dd, J=7.9, 1.6 Hz, 1H), 7.58 (s, 1H), 7.23 (ddd, J=8.5, 7.2, 1.6 Hz, 1H), 6.91-6.83 (m, 4H), 6.03 (s, 3H), 4.88-4.64 (m, 2H), 3.50-3.32 (m, 2H), 3.06-2.95 (m, 2H), 2.24-2.06 (m, 2H), 1.97-1.91 (m, 2H).

### Step 5:

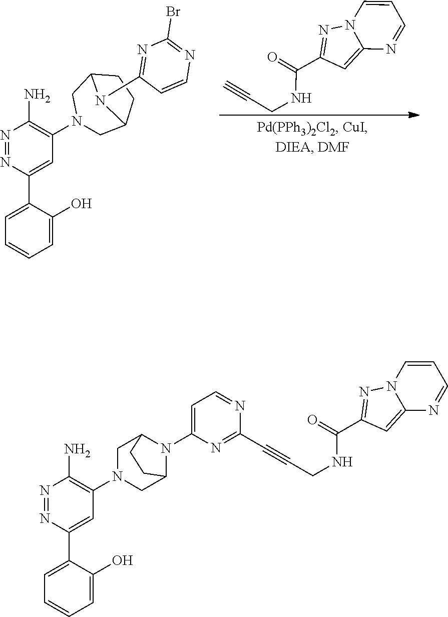

2-(6-amino-5-(8-(2-bromopyrimidin-4-yl)-3,8-diazabicyclo[3.2.1]octan-3-yl)pyridazin-3-yl)phenol (15 mg, 33 μmol, 1 eq), N-(but-3-yn-1-yl)pyrazolo[1,5-a]pyrimidine-2-carboxamide (66 μmol, 2 eq), and DIEA (12.8 mg, 99 μmol, 3 eq) were dissolved in anhydrous DMF (0.7 mL), and the resulting solution was degassed under an argon stream for 5 minutes. Palladium(II)bis(triphenylphosphine) dichloride (16.4 mg, 0.0233 mmol, 0.1 eq.) and CuI (2.5 mg, 13.2 μmol, 0.4 eq) were added. The reaction was stirred at 90° C. for 6 hours, and then cooled to room temperature. The solution was purified by prep-HPLC (Method A) to give N-(3-(4-(3-(3-amino-6-(2-hydroxyphenyl)pyridazin-4-yl)-3,8-diazabicyclo[3.2.1]octan-8-yl)pyrimidin-2-yl)prop-2-yn-1-yl)pyrazolo[1,5-a]pyrimidine-2-carboxamide.

### Example 3

### Preparation of (E)-N-(3-(4-(4-(3-amino-6-(2-hydroxyphenyl)pyridazin-4-yl)-1,4-diazepan-1-yl)pyrimidin-2-yl)allyl)quinoxaline-2-carboxamide (Compound P-110)

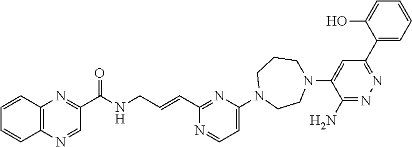 [SMILES: Nc1nnc(-c2ccccc2O)cc1N1CCCN(c2ccnc(/C=C/CNC(=O)c3cnc4ccccc4n3)n2)CC1]

### Step 1:

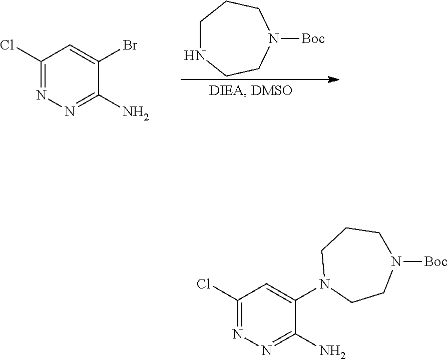 [SMILES: Nc1nnc(Cl)cc1Br ; CC(C)(C)OC(=O)N1CCCNCC1 ; CC(C)(C)OC(=O)N1CCCN(c2cc(Cl)nnc2N)CC1]

A mixture of 4-bromo-6-chloropyridazin-3-amine (2.07 g, 10.0 mmol, 2 eq), DIEA (6.45 g, 49.93 mmol, 8.70 mL, 10 eq), and tert-butyl 1,4-diazepane-1-carboxylate (1 g, 4.99 mmol, 980.39 μL, 1 eq) in DMSO (10 mL) was degassed and purged with N₂ three times. The mixture was stirred at 120° C. for 4 days under a N₂ atmosphere. The reaction mixture was concentrated under reduced pressure. The residue provided was diluted with water (20 mL) and extracted with ethyl acetate (2×10 mL). The combined organic layers were washed with brine (10 mL), dried over Na₂SO₄, filtered, and concentrated under reduced pressure to give a residue. The residue was purified by column chromatography (SiO₂, 1 to 100% ethyl acetate in petroleum ether) to give tert-butyl 4-(3-amino-6-chloropyridazin-4-yl)-1,4-diazepane-1-carboxylate. ¹H NMR (400 MHz, CDCl₃) δ 6.76 (s, 1H), 5.07 (s, 1H), 4.79 (d, J=2.0 Hz, 1H), 3.71-3.46 (m, 4H), 3.37-3.18 (m, 411), 2.04-1.86 (m, 2H), 1.47 (s, 911).

### Step 2:

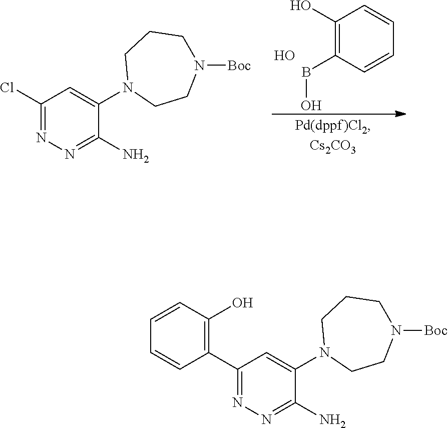

A mixture of tert-butyl 4-(3-amino-6-chloropyridazin-4-yl)-1,4-diazepane-1-carboxylate (8 g, 24.40 mmol, 1 eq), (2-hydroxyphenyl)boronic acid (5.05 g, 36.61 mmol, 1.5 eq), Cs₂CO₃ (47.71 g, 146.43 mmol, 6 eq), and Pd(dppf)Cl₂ (1.79 g, 2.44 mmol, 0.1 eq) in dioxane (100 mL) and water (20 mL) was degassed and purged with N₂ three times. The mixture was stirred at 80° C. for 12 hours under a N₂ atmosphere. The reaction mixture was quenched by addition of water (200 mL) and extracted with ethyl acetate (3×100 mL). The combined organic layers were washed with brine (2×50 mL), dried over Na₂SO₄, filtered, and concentrated under reduced pressure to give a residue. The residue was purified by column chromatography (SiO₂, 1 to 100% ethyl acetate in petroleum ether) to give tert-butyl 4-(3-amino-6-(2-hydroxyphenyl)pyridazin-4-yl)-1,4-diazepane-1-carboxylate. ¹H NMR (400 MHz, CDCl₃) δ 14.04-13.51 (m, 1H), 7.62-7.56 (m, 1H), 7.39-7.28 (m, 2H), 7.05 (d, J=8.0 Hz, 1H), 6.91 (t, J=7.6 Hz, 1H), 5.02 (s, 1H), 4.79 (s, 1H), 3.74-3.52 (m, 4H), 3.44-3.26 (m, 411), 2.11-1.90 (m, 2H), 1.48 (s, 911).

### Step 3:

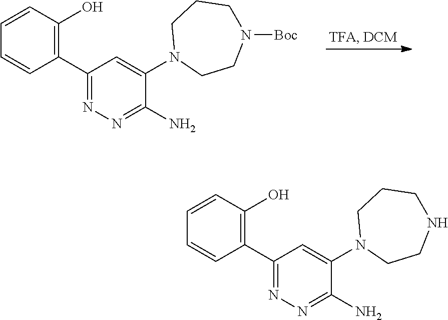 [SMILES: CC(C)(C)OC(=O)N1CCCN(c2cc(-c3ccccc3O)nnc2N)CC1 ; Nc1nnc(-c2ccccc2O)cc1N1CCCNCC1]

A mixture of tert-butyl 4-(3-amino-6-(2-hydroxyphenyl)pyridazin-4-yl)-1,4-diazepane-1-carboxylate (8 g, 20.75 mmol, 1 eq) and TFA (15.40 g, 135.06 mmol, 10 mL, 6.51 eq) in DCM (80 mL) was prepared. The mixture was stirred at 20° C. for 2 hours. The reaction mixture was concentrated in vacuo. The crude product was triturated with MTBE (20 mL) to give 2-(6-amino-5-(1,4-diazepan-1-yl)pyridazin-3-yl)phenol. ¹H NMR (400 MHz, d₄-MeOD) δ 7.58 (J=1.6, 8.0 Hz, 1H), 7.49 (s, 1H), 7.47-7.41 (m, 1H), 7.08-7.01 (m, 2H), 4.04-3.96 (m, 2H), 3.82-3.74 (m, 2H), 3.58-3.50 (m, 2H), 3.47-3.39 (m, 2H), 2.29 (quin, J=5.6 Hz, 2H).

### Step 4:

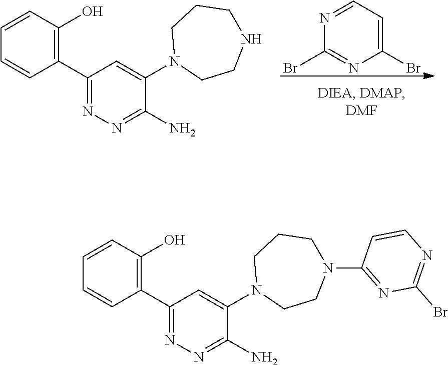 [SMILES: Nc1nnc(-c2ccccc2O)cc1N1CCCNCC1 ; Brc1ccnc(Br)n1 ; Nc1nnc(-c2ccccc2O)cc1N1CCCN(c2ccnc(Br)n2)CC1]

A mixture of 2-(6-amino-5-(1,4-diazepan-1-yl)pyridazin-3-yl)phenol (3.57 g, 15.02 mmol, 1.5 eq), DIEA (6.47 g, 50.08 mmol, 8.72 mL, 5 eq), and DMAP (122.36 mg, 1.00 mmol, 0.1 eq) in DMF (40 mL) was degassed and purged with N₂ three times. The mixture was stirred at 20° C. for 12 hours under a N₂ atmosphere. The reaction mixture was filtered. The residue was purified by prcp-HPLC (column: Welch Xtimate C₁₈ 250×70 mm×10 μm; mobile phase: (water (NH₄HCO₃)-ACN); B %: 33%-63%, 20 min) to give 2-(6-amino-5-(4-(2-bromopyrimidin-4-yl)-1,4-diazepan-1-yl)pyridazin-3-yl)phenol. ¹H NMR (400 MHz, d₄-MeOD) δ 7.90 (s, 1H), 7.69 (d, J=8.0 Hz, 1H), 7.52 (s, 1H), 7.28-7.20 (m, 1H), 6.95-6.88 (m, 2H), 6.78-6.64 (m, 1H), 4.22-4.09 (m, 1H), 4.07-3.94 (m, 111), 3.86-3.70 (m, 2H), 3.62-3.50 (m, 2H), 3.44-3.35 (m, 2H), 2.21-2.09 (m, 2H).

### Step 5:

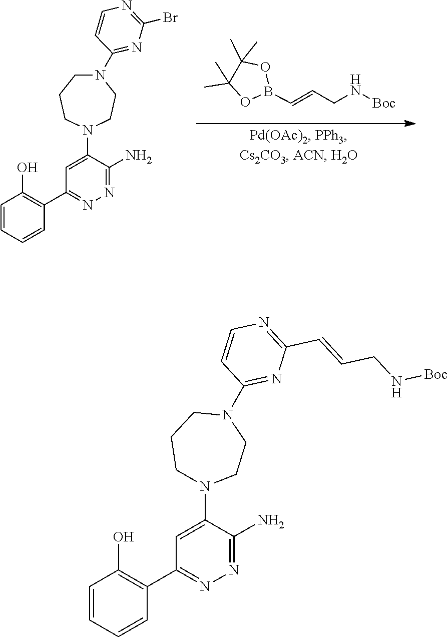 [SMILES: Nc1nnc(-c2ccccc2O)cc1N1CCCN(c2ccnc(Br)n2)CC1 ; CC(C)(C)OC(=O)NC/C=C/B1OC(C)(C)C(C)(C)O1 ; CC(C)(C)OC(=O)NC/C=C/c1nccc(N2CCCN(c3cc(-c4ccccc4O)nnc3N)CC2)n1]

A solution of 2-(6-amino-5-(4-(2-bromopyrimidin-4-yl)-1,4-diazepan-1-yl)pyridazin-3-yl)phenol (50.0 mg, 113 μmol, 1 eq.), tert-butyl (E)-(3-(4,4,5,5-tetramethyl-1,3,2-dioxaborolan-2-yl)allyl)carbamate (38.4 mg, 136 μmol, 1.2 eq.), triphenylphosphine (5.93 mg, 22.6 μmol, 0.2 eq.), and cesium carbonate (110 mg, 339 μmol, 3 eq.) in ACN (2 mL) and water (0.5 mL) was degassed under an argon stream for 5 minutes. Palladium(II)acetate (1.27 mg, 5.65 μmol, 0.05 eq.) was added. The reaction was stirred at 100° C. for 4 hours. The reaction was cooled to room temperature. The solution was purified by prep-HPLC (column: Phenomenex Luna C₁₈ 100×40 mm×5 mm; mobile phase: (0.1% formic acid in water-0.1% formic acid in ACN); B %: 5%-75%, 8 min). The fractions were combined and lyophilized to give tert-butyl (E)-(3-(4-(4-(3-amino-6-(2-hydroxyphenyl)pyridazin-4-yl)-1,4-diazepan-1-yl)pyrimidin-2-yl)allyl)carbamate, m/z (ESI⁺) 519 (M+H)⁺.

### Step 6:

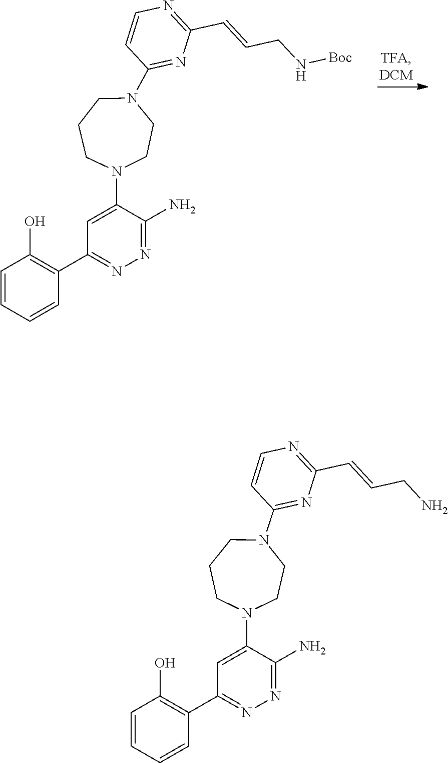 [SMILES: CC(C)(C)OC(=O)NC/C=C/c1nccc(N2CCCN(c3cc(-c4ccccc4O)nnc3N)CC2)n1 ; NC/C=C/c1nccc(N2CCCN(c3cc(-c4ccccc4O)nnc3N)CC2)n1]

To a solution of tert-butyl (E)-(3-(4-(4-(3-amino-6-(2-hydroxyphcnyl)pyridazin-4-yl)-1,4-diazepan-1-yl)pyrimidin-2-yl)allyl)carbamate (55.8 mg, 108 μmol, 1 eq) in DCM (2 mL), was added TFA (1 mL). The mixture was stirred at 20° C. for 1.5 hours. The reaction mixture was concentrated in vacuo. The crude product was triturated with diethyl ether (20 mL) to give (E)-2-(6-amino-5-(4-(2-(3-aminoprop-1-en-1-yl)pyrimidin-4-yl)-1,4-diazepan-1-yl)pyridazin-3-yl)phenol, which was used directly in the next step.

### Step 7:

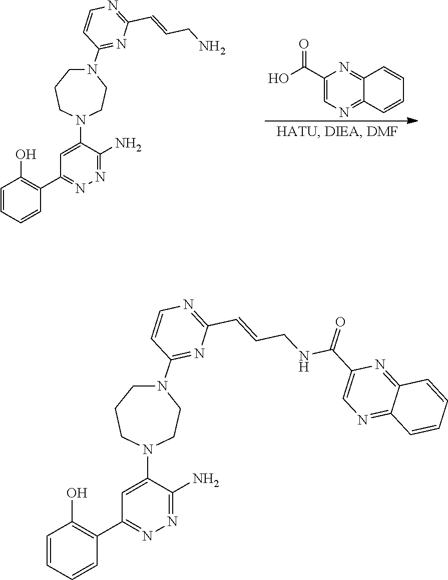 [SMILES: NC/C=C/c1nccc(N2CCCN(c3cc(-c4ccccc4O)nnc3N)CC2)n1 ; O=C(O)c1cnc2ccccc2n1 ; Nc1nnc(-c2ccccc2O)cc1N1CCCN(c2ccnc(/C=C/CNC(=O)c3cnc4ccccc4n3)n2)CC1]

To a solution of quinoxaline-2-carboxylic acid (6.44 mg, 371 μmol, 1 eq) in DMF (1.0 mL), was added (E)-2-(6-amino-5-(4-(2-(3-aminoprop-1-en-1-yl)pyrimidin-4-yl)-1,4-diazepan-1-yl)pyridazin-3-yl)phenol (23.9 mg, 37 μmol, 1 eq), HATU (14.1 mg, 37 μmol, 1 eq), and DIEA (19.1 mg, 25.8 μL, 148 mol, 4 eq). The reaction was stirred at 25° C. for 16 hrs. The solution was purified by prep-HPLC (column: Phenomenex Luna C₁₈ 100×40 mm×5 mm; mobile phase: (0.1% formic acid in water-0.1% formic acid in ACN); B %: 10%-40%, 8 min). The fractions were combined and lyophilized to give (E)-N-(3-(4-(4-(3-amino-6-(2-hydroxyphenyl)pyridazin-4-yl)-1,4-diazepan-1-yl)pyrimidin-2-yl)allyl)quinoxaline-2-carboxamide, m/z (ESI⁺) 575 (M+H)⁺.

### Example 4

### Preparation of N-(3-(4-(4-(3-amino-6-(2-hydroxyphenyl)pyridazin-4-yl)-1,4-diazepan-1-yl)-6-ethoxypyrimidin-2-yl)prop-2-yn-1-yl)pyrazolo[1,5-a]pyrimidine-2-carboxamide (Compound P-124)

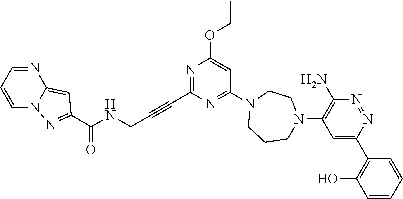 [SMILES: CCOc1cc(N2CCCN(c3cc(-c4ccccc4O)nnc3N)CC2)nc(C#CCNC(=O)c2cc3ncccn3n2)n1]

### Step 1:

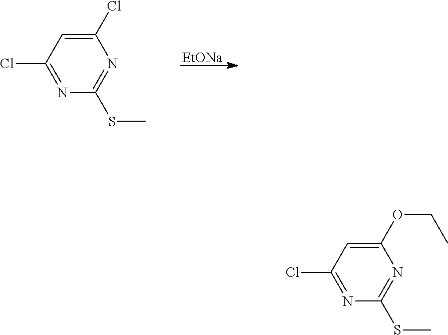

To a mixture of 4,6-dichloro-2-(methylthio)pyrimidine (6 g, 30.76 mmol, 1 eq) in ethanol (60 mL), was added sodium ethoxide (10.47 g, 30.76 mmol, 20% purity, 1 eq) in one portion at 20° C. under a N₂ atmosphere. The mixture was stirred at room temperature for 16 h. Saturated NH₄Cl solution (100 mL) was added. The aqueous layer was extracted with ethyl acetate (2×50 mL). The combined organic layers were washed with brine (100 mL), dried over Na₂SO₄, filtered, and concentrated under reduced pressure to give 4-chloro-6-ethoxy-2-(methylthio)pyrimidine, which was used into the next step without further purification. ¹H NMR (400 MHz, CDCl₃) δ 6.39 (s, 1H) 4.43 (q. J=7.2 Hz, 2H) 2.54 (s, 3H) 1.39 (t, J=7.2 Hz, 3H).

### Step 2:

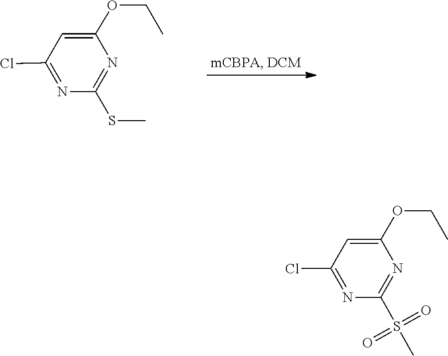

To a mixture of 4-chloro-6-ethoxy-2-(methylthio)pyrimidine (5.81 g, 28.39 mmol, 1 eq) in DCM (60 mL), was added mCPBA (48.98 g, 283.86 mmol, 10 eq) in one portion. The mixture was stirred at room temperature for 16 h. Saturated sodium thiosulfate solution (40 mL) was added. The organic layer was washed with brine (40 mL), dried over Na₂SO₄, filtered, and concentrated under reduced pressure to give a 4-chloro-6-ethoxy-2-(methylsulfonyl)pyrimidine, which was used into the next step without further purification, m/z (ESI⁺) 237.1 (M+H)⁺.

### Step 3:

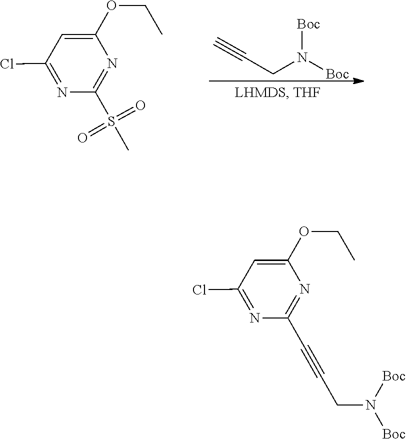 [SMILES: CCOc1cc(Cl)nc(S(C)(=O)=O)n1 ; C#CCN(C(=O)OC(C)(C)C)C(=O)OC(C)(C)C ; CCOc1cc(Cl)nc(C#CCN(C(=O)OC(C)(C)C)C(=O)OC(C)(C)C)n1]

To a solution of 4-chloro-6-ethoxy-2-(methylsulfonyl)pyrimidine (1 g, 4.23 mmol, 1 eq) in THF (20 mL) at −78° C., was added LiHMDS (1 M, 5.07 mL, 99.5% purity, 1.2 eq) dropwise over 15 min. The mixture was stirred at −78° C. for 30 min. Tert-butyl N-tert-butoxycarbonyl-N-prop-2-ynyl-carbamate (1.08 g, 4.23 mmol, 1 eq) in THF (20 mL) was added dropwise. The resulting mixture was stirred at −78° C. for 2 h. Saturated NH₄Cl solution (20 mL) was added. The aqueous layer was extracted with ethyl acetate (2×10 mL). The combined organic layers were washed with brine (20 mL), dried over Na₂SO₄, filtered, and concentrated under reduced pressure to give a residue. The residue was purified by prep-HPLC (method A) to give di-tert-butyl (3-(4-chloro-6-ethoxypyrimidin-2-yl)prop-2-yn-1-yl)iminodicarbonate. ¹H NMR (400 MHz, CDCl₃) δ 6.67 (s, 1H) 4.63 (s, 2H) 4.44 (q, J=7.2 Hz, 2H) 1.63 (br s, 3H) 1.55 (s, 18H).

### Step 4:

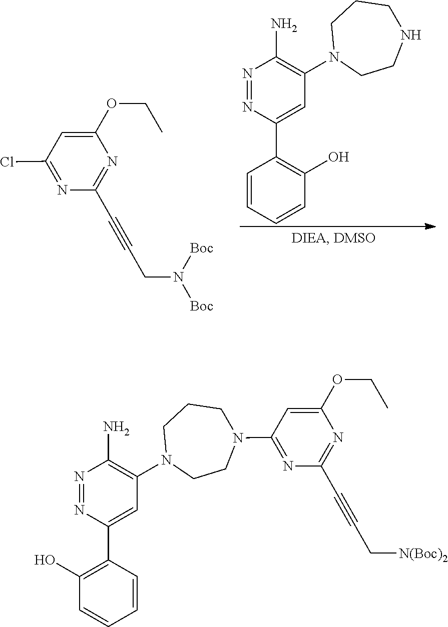

To a solution of di-tert-butyl (3-(4-chloro-6-ethoxypyrimidin-2-yl)prop-2-yn-1-yl)iminodicarbonate (150 mg, 364.18 μmol, 1 eq) in DMSO (5 mL), was added dropwise 2-[6-amino-5-(1,4-diazepan-1-yl)pyridazin-3-yl]phenol (103.92 mg, 364.18 μmol, 1 eq) at 50° C. over 15 min. After addition, the mixture was stirred at 50° C. for 30 min, and then DIEA (470.67 mg, 3.64 mmol, 634.33 μL, 10 eq) was added dropwise. The resulting mixture was stirred at 50° C. for 2 hours. Saturated NH₄Cl solution (1 mL) was added. The aqueous layer was extracted with ethyl acetate (2×0.5 mL). The combined organic layers were washed with brine (1 mL), dried over Na₂SO₄, filtered, and concentrated under reduced pressure to give tert-butyl (3-(4-(4-(3-amino-6-(2-hydroxyphenyl)pyridazin-4-yl)-1,4-diazepan-1-yl)-6-ethoxypyrimidin-2-yl)prop-2-yn-1-yl)(tert-butoxycarbonyl)carbamate. 1H NMR (400 MHz, CDCl₃) δ 7.51-7.55 (m, 1H) 7.32-7.34 (m, 1H) 7.28-7.31 (m, 1H) 7.04-7.07 (m, 1H) 6.88-6.93 (m, 1H) 4.33-4.39 (m, 2H) 5.74 (s, 1H) 4.13 (q, J=7.2 Hz, 2H) 3.96-4.09 (m, 2H) 3.67-3.79 (m, 2H) 3.45 (br t, J=5.2 Hz, 2H) 3.30-3.35 (m, 2H) 2.09-2.13 (m, 2H) 1.56 (s, 18H) 1.41 (s, 3H).

### Step 5:

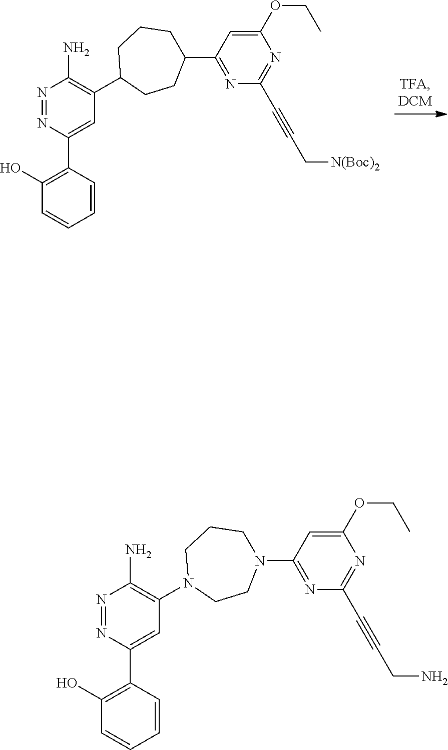

To a solution of tert-butyl (3-(4-(4-(3-amino-6-(2-hydroxyphenyl)pyridazin-4-yl)-1,4-diazepan-1-yl)-6-ethoxypyrimidin-2-yl)prop-2-yn-1-yl)(tert-butoxycarbonyl)carbamate (50 mg, 75.67 μmol, 1 eq) in DCM (1 mL), was added TFA (1 M, 378.35 μL, 5 eq). The mixture was stirred at room temperature for 1 h. The solvents were evaporated in vacuo to give 2-(6-amino-5-(4-(2-(3-aminoprop-1-yn-1-yl)-6-ethoxypyrimidin-4-yl)-1,4-diazepan-1-yl)pyridazin-3-yl)phenol, m/z (ESI⁺) 459.2 (M−1)⁺.

### Step 6:

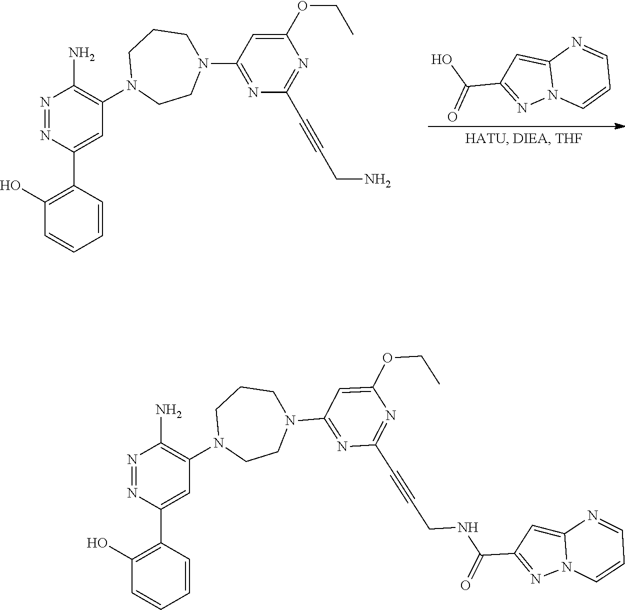 [SMILES: CCOc1cc(N2CCCN(c3cc(-c4ccccc4O)nnc3N)CC2)nc(C#CCN)n1 ; O=C(O)c1cc2ncccn2n1 ; CCOc1cc(N2CCCN(c3cc(-c4ccccc4O)nnc3N)CC2)nc(C#CCNC(=O)c2cc3ncccn3n2)n1]

To a solution of pyrazolo[1,5-a]pyrimidine-2-carboxylic acid (7.08 mg, 43.43 μmol, 1 eq) in DMF (1 mL), was added 2-(6-amino-5-(4-(2-(3-aminoprop-1-yn-1-yl)-6-ethoxypyrimidin-4-yl)-1,4-diazepan-1-yl)pyridazin-3-yl)phenol (20 mg, 43.43 μmol, 1 eq), HATU (16.51 mg, 43.43 μmol, 1 eq), and DIEA (11.23 mg, 86.86 μmol, 15.13 μL, 2 eq). The mixture was stirred at 0° C. for 1 hour. Saturated NH₄Cl solution (1 mL) was added. The aqueous layer was extracted with ethyl acetate (2×0.5 mL). The combined organic layers were washed with brine (1 mL), dried over Na₂SO₄, filtered, and concentrated under reduced pressure to give a residue. The residue was purified by prep-HPLC (method A) to give N-(3-(4-(4-(3-amino-6-(2-hydroxyphenyl)pyridazin-4-yl)-1,4-diazepan-1-yl)-6-ethoxypyrimidin-2-yl)prop-2-yn-1-yl)pyrazolo[1,5-a]pyrimidine-2-carboxamide. ¹H NMR (400 MHz, d₄-MeOD) δ 14.23-14.28 (m, 1H) 9.16-9.21 (m, 1H) 9.13-9.16 (m, 1H) 8.66 (dd, J=4.0, 1.6 Hz, 1H) 7.81-7.87 (m, 1H) 7.49 (s, 1H) 7.23 (s, 2H) 6.82-6.88 (m, 2H) 6.17 (s, 1H) 5.96 (s, 1H) 4.33 (d, J=6.0 Hz, 2H) 4.22 (q, J=6.8 Hz, 2H) 3.37-3.43 (m, 2H) 3.31 (s, 2H) 3.23 (br dd, J=5.2, 4.4 Hz, 2H) 3.16 (d, J=5.2 Hz, 1H) 2.02 (br s, 2H) 1.24 (t, J=7.2 Hz, 3H).

### Example 5

### Preparation of 2-(5-(4-(2-(3-(1H-benzo[d]imidazol-1-yl)prop-1-yn-1-yl)pyrimidin-4-yl)-1,4-diazepan-1-yl)-6-aminopyridazin-3-yl)phenol (Compound P-150)

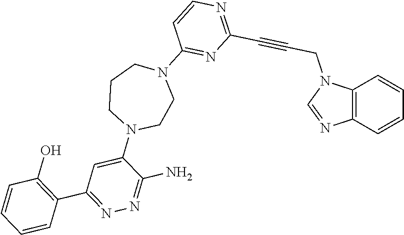 [SMILES: Nc1nnc(-c2ccccc2O)cc1N1CCCN(c2ccnc(C#CCn3cnc4ccccc43)n2)CC1]

### Step 1:

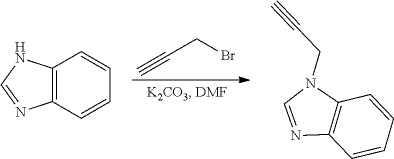

To a solution of 3-bromoprop-1-yne (80%, 3.78 g, 25.39 mmol, 2.74 mL, 3.0 eq) in DMF (10 mL), was added K₂CO₃ (2.34 g, 16.93 mmol, 2.0 eq) and 1H-benzo[d]imidazole (1.00 g, 8.46 mmol, 1 eq). The mixture was stirred at 25° C. for 12 hours. Water (30 mL) was added, and the resulting mixture was extracted with ethyl acetate (2×60 mL). The combined organic layers were washed with brine (30 mL), dried over anhydrous Na₂SO₄, filtered, and concentrated to give a residue. The residue was purified by prep-HPLC (method A) to give 1-(prop-2-yn-1-yl)-1H-benzo[d]imidazole. ¹H NMR (400 MHz, CDCl₃) δ 8.01 (s, 1H), 7.84-7.79 (m, 1H), 7.50-7.45 (m, 1H), 7.37-7.28 (m, 2H), 4.91 (d, J=2.8 Hz, 2H), 2.49 (t, J=2.4 Hz, 1H).

### Step 2:

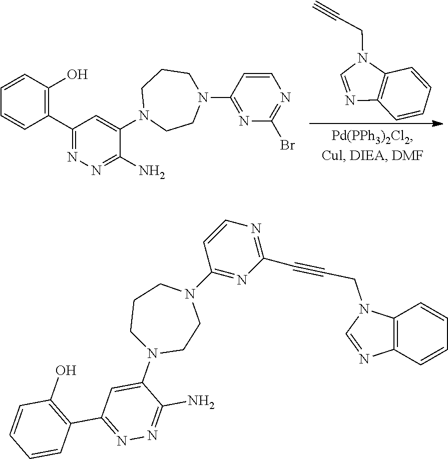 [SMILES: Nc1nnc(-c2ccccc2O)cc1N1CCCN(c2ccnc(Br)n2)CC1 ; C#CCn1cnc2ccccc21 ; Nc1nnc(-c2ccccc2O)cc1N1CCCN(c2ccnc(C#CCn3cnc4ccccc43)n2)CC1]

A mixture of 2-(6-amino-5-(4-(2-bromopyrimidin-4-yl)-1,4-diazepan-1-yl)pyridazin-3-yl)phenol (35.31 mg, 226.08 μmol, 2.0 eq), 1-(prop-2-yn-1-yl)-1H-benzo[d]imidazole (50 mg, 113.04 μmol, 1 eq), Pd(PPh₃)₂Cl₂ (7.93 mg, 11.30 μmol, 0.1 eq), CuI (6.46 mg, 33.91 μmol, 0.3 eq), and DIEA (51.13 mg, 395.65 μmol, 68.91 μL, 3.5 eq) in DMF (0.5 mL) was degassed and purged with nitrogen. The mixture was stirred at 70° C. for 1 hour under a N₂ atmosphere. The reaction mixture was filtered and the filtrate was purified by prep-HPLC (method A) to give 2-(5-(4-(2-(3-(1H-benzo[d]imidazol-1-yl)prop-1-yn-1-yl)pyrimidin-4-yl)-1,4-diazepan-1-yl)-6-aminopyridazin-3-yl)phenol. ¹H NMR (400 MHz, d₄-CD₃OD) δ 8.32 (d, J=3.2 Hz, 1H), 7.99 (d, J=0.8 Hz, 1H), 7.78-7.67 (m, 2H), 7.59 (d, J=8.0 Hz, 1H), 7.41 (s, 1H), 7.38-7.28 (m, 2H), 7.19 (t, J=7.6 Hz, 1H), 6.88 (d, J=8.0 Hz, 1H), 6.85-6.80 (m, 1H), 6.68-6.60 (m, 1H), 5.40 (s, 2H), 4.16-3.87 (m, 2H), 3.83-3.61 (m, 2H), 3.58-3.41 (m, 2H), 3.33 (d, J=3.6 Hz, 2H), 2.13-2.05 (m, 2H).

### Example 6

### Preparation of o-{5-[(R)-4-{2-[(E)-3-(dimethylamino)-1-propenyl]-4-pyrimidinyl}-5-methyl-1,4-diazepan-1-yl]-6-amino-3-pyridazinyl}phenol (Compound P-218)

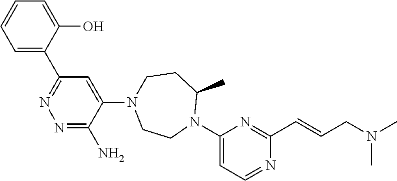 [SMILES: C[C@@H]1CCN(c2cc(-c3ccccc3O)nnc2N)CCN1c1ccnc(/C=C/CN(C)C)n1]

### Step 1:

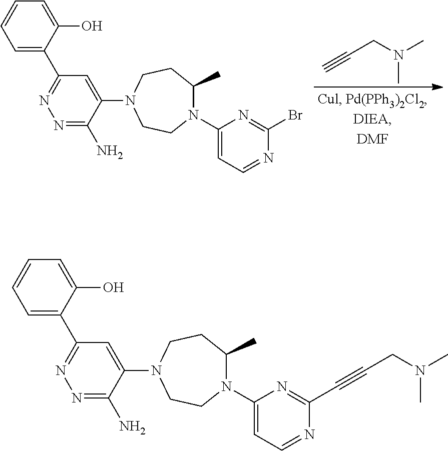

To a solution of (R)-2-(6-amino-5-(4-(2-bromopyrimidin-4-yl)-5-methyl-1,4-diazepan-1-yl)pyridazin-3-yl)phenol (350 mg, 766.98 μmol, 1 eq), N,N-dimethylprop-2-yn-1-amine (191.28 mg, 2.30 mmol, 243.98 μL, 3 eq), DIEA (346.94 mg, 2.68 mmol, 467.58 μL, 3.5 eq), CuI (43.82 mg, 230.09 μmol, 0.3 eq) in DMF (3 mL) was added Pd(PPh₃)₂Cl₂ (53.83 mg, 76.70 μmol, 0.1 eq) at 25° C. under N₂. The reaction mixture was degassed and purged with N₂ three times. The reaction mixture was stirred at 70° C. for 1 hour under a N₂ atmosphere. The reaction mixture was partitioned between ethyl acetate (5 mL) and H₂O (5 mL). The organic phase was separated, washed with brine (3 mL), dried over Na₂SO₄, filtered and concentrated under reduced pressure to give (R)-2-(6-amino-5-(4-(2-(3-(dimethylamino)prop-1-yn-1-yl)pyrimidin-4-yl)-5-methyl-1,4-diazepan-1-yl)pyridazin-3-yl)phenol which was used in the following step directly. ¹H NMR (400 MHz, CD₃OD) δ 8.15-7.99 (m, 1H), 7.83-7.68 (m, 1H), 7.55-7.45 (m, 1H), 7.28-7.15 (m, 1H), 7.00-6.85 (m, 2H), 6.74 (d, J=6.0 Hz, 1H), 4.77-4.54 (m, 1H), 4.16-3.99 (m, 1H), 3.92-3.75 (m, 1H), 3.69-3.54 (m, 2H), 3.53 (s, 2H), 3.38 (s, 1H), 3.31 (d, J=3.2 Hz, 1H), 3.22-3.00 (m, 1H). 2.92-2.72 (m, 1H), 2.40 (s, 6H), 1.32-1.20 (m, 4H).

### Step 2:

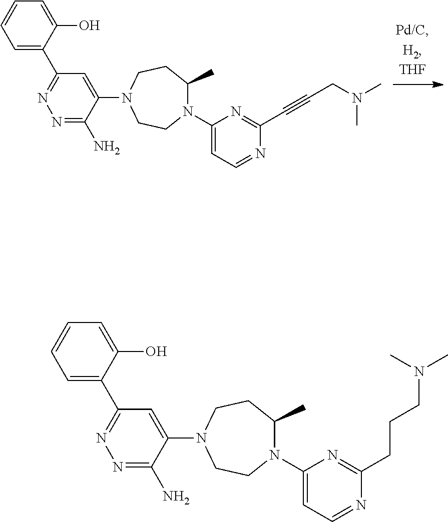 [SMILES: C[C@@H]1CCN(c2cc(-c3ccccc3O)nnc2N)CCN1c1ccnc(C#CCN(C)C)n1 ; C[C@@H]1CCN(c2cc(-c3ccccc3O)nnc2N)CCN1c1ccnc(CCCN(C)C)n1]

To a solution of (R)-2-(6-amino-5-(4-(2-(3-(dimethylamino)prop-1-yn-1-yl)pyrimidin-4-yl)-5-methyl-1,4-diazepan-1-yl)pyridazin-3-yl)phenol (10 mg, 21.81 μmol, 1 eq) in THF (0.5 mL) was added Pd/C (5 mg, 21.81 μmol, 60% wt. %, 1 eq) at 25° C. under an argon atmosphere. The reaction mixture was stirred 25° C. for 1 hour under a H₂ atmosphere. The reaction mixture was filtered, and the filtrate was concentrated under reduced pressure to give a residue. The residue was purified by prep-HPLC (Phenomenex Luna C₁₈ 75 mm×30 mm×3 μm; Mobile phase: [water (+1% formic acid)-ACN]; B %: 1%-30%, 8 min gradient) to give o-{5-[(R)-4-{2-[(E)-3-(dimethylamino)-1-propenyl]-4-pyrimidinyl}-5-methyl-1,4-diazepan-1-yl]-6-amino-3-pyridazinyl}phenol. ¹H NMR (400 MHz, CD₃OD) δ 8.50-8.45 (m, 1H), 8.13-8.12 (m, 1H), 7.80-7.72 (m, 1H), 7.52-7.49 (m, 1H), 7.30-7.26 (m, 1H), 6.92-6.85 (m, 2H), 6.80-6.61 (m, 1H), 4.13-4.12 (m, 1H), 3.87-3.84 (m, 2H), 3.63-3.60 (m, 5H), 3.25-3.19 (m, 3H), 3.15-3.10 (m, 6H), 2.40-2.19 (m, 4H), 1.36-1.28 (m, 3H).

### Example 7

### Preparation of o-[6-amino-5-(3-{2-[3-(dimethylamino)-1-propynyl]-4-pyridyloxy}-1-azetidinyl)-3-pyridazinyl]phenol (Compound P-387)

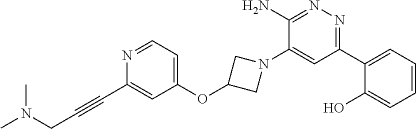 [SMILES: CN(C)CC#Cc1cc(OC2CN(c3cc(-c4ccccc4O)nnc3N)C2)ccn1]

### Step 1:

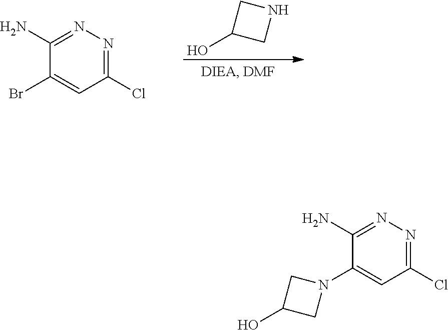 [SMILES: Nc1nnc(Cl)cc1Br ; OC1CNC1 ; Nc1nnc(Cl)cc1N1CC(O)C1]

A solution of 4-bromo-6-chloropyridazin-3-amine (5 g, 23.99 mmol, 1 eq), azetidin-3-ol (2.63 g, 23.99 mmol, 1 eq, HCl) and DIEA (31.00 g, 239.87 mmol, 41.78 mL, 10 eq) in DMF (50 mL) was degassed and purged with N₂, and the mixture was stirred at 120° C. for 12 hours under a N₂ atmosphere. The reaction mixture was concentrated under reduced pressure to give a residue. The residue was purified by column chromatography (SiO₂, 0 to 10% methanol in ethyl acetate) to give 1-(3-amino-6-chloropyridazin-4-yl)azetidin-3-ol. ¹H NMR (400 MHz, d₆-DMSO) δ 6.32 (s, 1H), 5.73 (s, 2H), 5.68 (d, J=6.4 Hz, 1H), 4.52-4.46 (m, 1H), 4.35-4.28 (m, 2H), 3.73 (dd, J=4.8, 9.2 Hz, 2H).

### Step 2:

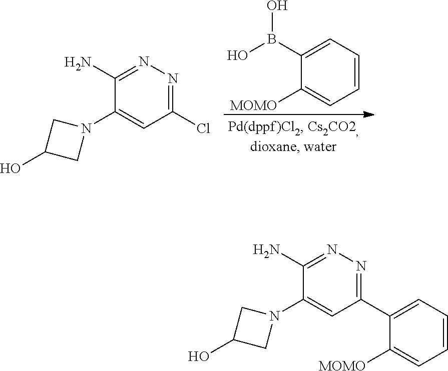

A mixture of 1-(3-amino-6-chloropyridazin-4-yl)azetidin-3-ol (4.4 g, 21.93 mmol, 1 eq), [2-(methoxymethoxy)phenyl]boronic acid (4.39 g, 24.12 mmol, 1.1 eq), Pd(dppf)Cl₂ (1.60 g, 2.19 mmol, 0.1 eq) and Cs₂CO₃ (42.87 g, 131.59 mmol, 6 eq) in dioxane (45 mL) and water (22.5 mL) was degassed and purged with N₂ three times. The mixture was stirred at 80° C. for 5 hours under a N₂ atmosphere. The residue was diluted with water (50 mL) and extracted with ethyl acetate (3×50 mL). The combined organic layers were washed with brine (2×50 mL), dried over Na₂SO₄, filtered and the filtrate was concentrated under reduced pressure to give a residue. The residue was triturated with ACN (20 mL) to give 1-(3-amino-6-(2-(methoxymethoxy)phenyl)pyridazin-4-yl)azetidin-3-ol. ¹H NMR (400 MHz, d₆-DMSO) δ 7.59 (d, J=7.6 Hz, 1H), 7.34 (t, J=7.6 Hz, 1H), 7.17 (d, J=8.0 Hz, 1H), 7.07 (t, J=7.6 Hz, 1H), 6.65 (s, 1H), 5.68 (s, 1H), 5.61 (s, 2H), 5.20 (s, 2H), 4.50 (d, J=6.0 Hz, 1H), 4.28 (t, J=7.2 Hz, 2H), 3.63 (t, J=6.4 Hz, 2H), 3.38-3.36 (m, 3H).

### Step 3:

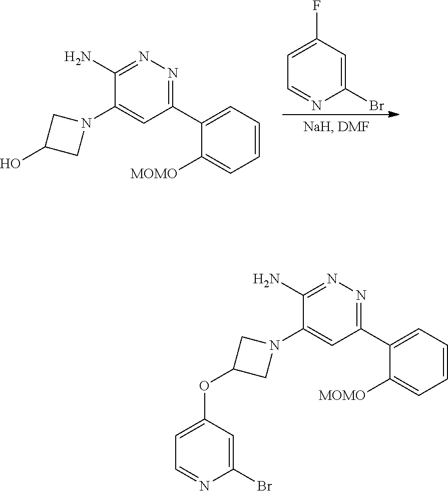

To a solution of 1-(3-amino-6-(2-(methoxymethoxy)phenyl)pyridazin-4-yl)azetidin-3-ol (1 g, 3.31 mmol, 1 eq) in DMF (10 mL) was added NaH (661.47 mg, 16.54 mmol, 60% purity, 5 eq) in portions at 0° C. under N₂. After 10 mins, 2-bromo-4-fluoro-pyridine (582.10 mg, 3.31 mmol, 1 eq) in DMF (5 mL) was added dropwise to the solution at 0° C. The mixture was stirred at 20° C. for 1 hour. Saturated NH₄Cl solution (10 mL) was added and the reaction extracted with ethyl acetate (3×10 mL). The combined organic layers were washed with brine (2×10 mL), dried over Na₂SO₄, filtered and the filtrate was concentrated under reduced pressure to give a residue. The residue was triturated with ACN (5 mL) to give 4-(3-((2-bromopyridin-4-yl)oxy)azetidin-1-yl)-6-(2-(methoxymethoxy)phenyl)pyridazin-3-amine. ¹H NMR (400 MHz, d₆-DMSO) δ 8.24 (d, J=6.0 Hz, 1H), 7.59 (dd, J=1.6, 7.6 Hz, 1H), 7.39-7.29 (m, 1H), 7.21-7.15 (m, 2H), 7.08 (dt, J=1.2, 7.6 Hz, 1H), 7.01 (dd, J=2.4, 5.6 Hz, 1H), 6.73 (s, 1H), 5.66 (s, 2H), 5.23 (td, J=2.8, 6.4 Hz, 1H), 5.19 (s, 2H), 4.49 (dd, J=6.4, 9.6 Hz, 2H), 4.08-4.01 (m, 2H), 3.33 (s, 3H).

### Step 4:

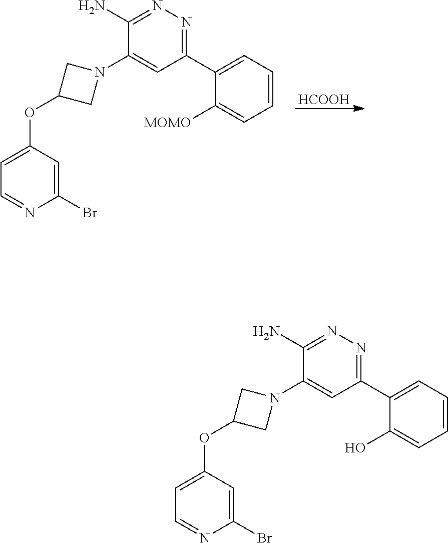

A solution of 4-(3-((2-bromopyridin-4-yl)oxy)azetidin-1-yl)-6-(2-(methoxymethoxy)phenyl)pyridazin-3-amine (140 mg, 305.47 μmol, 1 eq) in HCOOH (1.4 mL) was stirred at 20° C. for 5 hours. Saturated NaHCO₃ solution (5 mL) was added and the mixture was extracted with ethyl acetate (3×2 mL). The combined organic layers were washed with brine (2×2 mL), dried over Na₂SO₄, filtered and concentrated under reduced pressure to give a residue. The residue was purified by prep-HPLC to give 4-(3-((2-bromopyridin-4-yl)oxy)azetidin-1-yl)-6-(2-(methoxymethoxy)phenyl)pyridazin-3-amine. ¹H NMR (400 MHz, d₆-DMSO) δ 14.62 (s, 1H), 8.25 (d, J=5.6 Hz, 1H), 7.92 (dd, J=1.6, 8.4 Hz, 1H), 7.25-7.18 (m, 2H), 7.06-7.01 (m, 2H), 6.88-6.83 (m, 2H), 5.97 (s, 2H), 5.27 (dt, J=3.6, 6.4 Hz, 1H), 4.65 (dd, J=6.4, 9.6 Hz, 2H), 4.22 (dd, J=3.2, 9.6 Hz, 2H)

### Step 5:

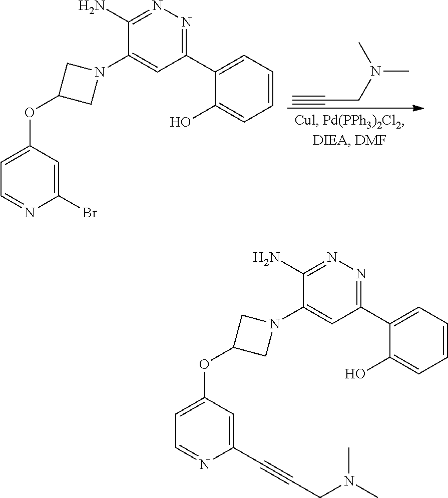

A mixture of 4-(3-((2-bromopyridin-4-yl)oxy)azetidin-1-yl)-6-(2-(methoxymethoxy)phenyl)pyridazin-3-amine (20 mg, 48.28-4ol, 1 eq), N,N-dimethylprop-2-yn-1-amine (12.04 mg, 144.84 μmol, 15.36 μL, 3 eq), CuI (2.76 mg, 14.48 μmol, 0.3 eq), DIEA (31.20 mg, 241.40 μmol, 42.05 μL, 5 eq) and Pd(PPh₃)₂Cl₂ (6.78 mg, 9.66 μmol, 0.2 eq) in DMF (0.2 mL) was degassed and purged with N₂ three times. The mixture was stirred at 40° C. for 2 hours under a N₂ atmosphere. The reaction mixture was filtered and the filtrate was purified by prep-HPLC (Phenomenex Luna C₁₈ 75 mm×30 mm×3 μm; Mobile phase: [water (+1% formic acid)-ACN]; B %: 1%-30%, 8 min gradient) to give o-[6-amino-5-(3-{2-[3-(dimethylamino)-1-propynyl]-4-pyridyloxy}-1-azetidinyl)-3-pyridazinyl]phenol. ¹H NMR (400 MHz, d₆-DMSO) δ 14.97-13.52 (m, 1H), 8.40 (s, 1), 8.26-8.04 (m, 1H), 7.92 (d, J=7.6 Hz, 1H), 7.22 (t, J=7.6 Hz, 1H), 7.07-7.00 (m, 2m), 6.98-6.93 (m, 1H), 6.88-6.82 (m, 2H), 5.97 (s, 2H), 5.30-5.22 (m, 1H), 4.65 (dd, J=6.4, 9.6 Hz, 2H), 4.21 (dd, J=3.2, 9.6 Hz, 2H), 3.49 (s, 2H), 2.26 (s, 6H).

Each of the compounds set forth in Table 3 were prepared following the procedures set forth above.

TABLE 3

| Com- | | |
|---|---|---|
| pound | | |
| # | IUPAC Name | Analysis |
| P-1 | N-[3-(4-{4-[3-amino-6-(2- | m/z (ESI⁺) 512 (M + H)⁺. |
| | hydroxyphenyl)pyridazin-4- | |
| | yl]piperazin-1-yl}pyrimidin-2- | |
| | yl)prop-2-yn-1-yl]-5-methyl- | |
| | 1,2-oxazole-3-carboxamide | |
| P-2 | N-[3-(4-{4-[3-amino-6-(2- | m/z (ESI⁺) 539 (M + H)⁺. |
| | hydroxyphenyl)pyridazin-4- | |
| | yl]piperazin-1-yl}pyrimidin-2- | |
| | yl)prop-2-yn-1-yl]-5-ethyl-1- | |
| | methyl-1H-pyrazole-3- | |
| | carboxamide | |
| P-3 | N-[3-(4-{4-[3-amino-6-(2- | m/z (ESI⁺) 512 (M + H)⁺. |
| | hydroxyphenyl)pyridazin-4- | |
| | yl]piperazin-1-yl}pyrimidin-2- | |
| | yl)prop-2-yn-1-yl]-5-methyl- | |
| | 1,2-oxazole-4-carboxamide | |
| P-4 | N-[3-(4-{4-[3-amino-6-(2- | m/z (ESI⁺) 508 (M + H)⁺. |
| | hydroxyphenyl)pyridazin-4- | |
| | yl]piperazin-1-yl}pyrimidin-2- | |
| | yl)prop-2-yn-1-yl]pyridine-3- | |
| | carboxamide | |
| P-5 | N-[3-(4-{4-[3-amino-6-(2- | m/z (ESI⁺) 509 (M + H)⁺. |
| | hydroxyphenyl)pyridazin-4- | |
| | yl]piperazin-1-yl}pyrimidin-2- | |
| | yl)prop-2-yn-1-yl]pyridazine- | |
| | 3-carboxamide | |
| P-6 | N-[3-(4-{4-[3-amino-6-(2- | m/z (ESI⁺) 551 (M + H)⁺. |
| | hydroxyphenyl)pyridazin-4- | |
| | yl]piperazin-1-y1}pyrimidin-2- | |
| | yl)prop-2-yn-1-yl]-5- | |
| | cyclopropyl-1-methyl-1H- | |
| | pyrazole-3-carboxamide | |
| P-7 | N-[3-(4-{4-[3-amino-6-(2- | m/z (ESI⁺) 511 (M + H)⁺. |
| | hydroxyphenyl)pyridazin-4- | |
| | yl]piperazin-1-yl}pyrimidin- | |
| | 2-yl)prop-2-yn-1-yl]-1-methyl- | |
| | 1H-pyrazole-3-carboxamide | |
| P-8 | N-[3-(4-{4-[3-amino-6-(2- | m/z (ESI⁺) 539 (M + H)⁺. |
| | hydroxyphenyl)pyridazin-4- | |
| | yl]piperazin-1-yl}pyrimidin-2- | |
| | yl)prop-2-yn-1-yl]-1,4,5- | |
| | trimethyl-1H-pyrazole-3- | |
| | carboxamide | |
| P-9 | N-[3-(4-{4-[3-amino-6-(2- | m/z (ESI⁺) 583 (M + H)⁺. |
| | hydroxyphenyl)pyridazin-4- | |
| | yl]piperazin-1-yl}pyrimidin-2- | |
| | yl)prop-2-yn-1-yl]-1-(2,2,2- | |
| | trifluoroethyl)-1H-pyrazole-3- | |
| | carboxamidc | |
| P-10 | N-[3-(4-{4-[3-amino-6-(2- | m/z (ESI⁺) 547 (M + H)⁺. |
| | hydroxyphenyl)pyridazin-4- | |
| | yl]piperazin-1-yl}pyrimidin-2- | |
| | yl)prop-2-yn-1-y1]-1- | |
| | (difluoromethyl)-1H-pyrazole- | |
| | 3-carboxamide | |
| P-11 | N-[3-(4-{4-[3-amino-6-(2- | m/z (ESI⁺) 537 (M + H)⁺. |
| | hydroxyphenyl)pyridazin-4- | |
| | yl]piperazin-1-yl}pyrimidin-2- | |
| | yl)prop-2-yn-1-y1]-1- | |
| | cyclopropyl-1H-pyrazole-3- | |
| | carboxamide | |
| P-12 | N-[3-(4-{4-[3-amino-6-(2- | m/z (ESI⁺) 525 (M + H)⁺. |
| | hydroxyphenyl)pyridazin-4- | |
| | yl]piperazin-1-yl}pyrimidin-2- | |
| | yl)prop-2-yn-1-yl]-1,5- | |
| | dimethyl-1H-pyrazole-3- | |
| | carboxamide | |
| P-14 | (R)-N-(3-(4-(4-(3-amino-6-(2- | m/z (ESI⁺) 576 (M + H)⁺. |
| | hydroxyphenyl)pyridazin-4-yl)- | |
| | 7-methyl-1,4-diazepan-1-yl) | |
| | pyrimidin-2-yl)prop-2-yn-1-yl) | |
| | pyrazolo[1,5-a]pyrimidine-2- | |
| | carboxamide | |
| P-15 | 2-(6-amino-5-(4-(2-(3-(oxazol- | m/z (ESI⁺) 484 (M + H)⁺. |
| | 2-ylamino)prop-1-yn-1-yl) | |
| | pyrimidin-4-yl)-1,4-diazepan- | |
| | 1-yl)pyridazin-3-yl)phenol | |
| P-16 | N-(3-(4-((3S,5S)-4-(3-amino- | m/z (ESI⁺) 576 (M + H)⁺. |
| | 6-(2-hydroxyphenyl) | |
| | pyridazin-4-y1)-3,5- | |
| | dimethylpiperazin-1-yl) | |
| | pyrimidin-2-yl)prop-2-yn- | |
| | 1-yl)pyrazolo[1,5- | |
| | a]pyrimidine-2- | |
| | carboxamide | |
| P-17 | N-(3-(4-(6-(3-amino-6-(2- | m/z (ESI⁺) 574 (M + H)⁺. |
| | hydroxyphenyl)pyridazin-4-yl)- | |
| | 3,6-diazabicyclo[3.2.1]octan-3- | |
| | yl)pyrimidin-2-yl)prop-2-yn-1- | |
| | yl)pyrazolo[1,5-a]pyrimidine-2- | |
| | carboxamide | |
| P-18 | N-(3-(4-(6-(3-amino-6-(2- | m/z (ESI⁺) 560 (M + H)⁺. |
| | hydroxyphenyl)pyridazin-4-yl)- | |
| | 2,6-diazabicyclo[3.2.0]heptan-2- | |
| | yl)pyrimidin-2-y1)prop-2-yn-1- | |
| | yl)pyrazolo[1,5-a]pyrimidine-2- | |
| | carboxamide | |
| P-19 | N-(3-(4-(4-(3-amino-6-(2- | m/z (ESI⁺) 576 (M + H)⁺. |
| | hydroxyphenyl)pyridazin-4-yl)- | |
| | 5-methyl-1,4-diazepan-1-yl) | |
| | pyrimidin-2-yl)prop-2-yn-1-yl) | |
| | pyrazolo[1,5-a]pyrimidine-2- | |
| | carboxamide | |
| P-20 | N-(3-(4-(4-(3-amino-6-(2- | m/z (ESI⁺) 576 (M + H)⁺. |
| | hydroxyphenyl)pyridazin-4-yl)- | |
| | 2-methyl-1,4-diazepan-1-yl) | |
| | pyrimidin-2-yl)prop-2-yn-1-yl) | |
| | pyrazolo[1,5-a]pyrimidine- | |
| | 2-carboxamide | |
| P-21 | 2-(6-amino-5-(4-(2-(3-((1- | m/z (ESI⁺) 497 (M + H)⁺. |
| | methyl-1H-pyrazol-3-yl) | |
| | amino)prop-1-yn-1-yl) | |
| | pyrimidin-4-yl)-1,4-diazepan- | |
| | 1-yl)pyridazin-3-yl)phenol | |
| P-22 | N-(4-(4-(8-(3-amino-6-(2- | m/z (ESI⁺) 588 (M + H)⁺. |
| | hydroxyphenyl)pyridazin-4-yl)- | |
| | 3,8-diazabicyclo[3.2.1]octan-3- | |
| | yl)pyrimidin-2-yl)but-3-yn-1- | |
| | yl)pyrazolo[1,5-a]pyrimidinc- | |
| | 2-carboxamide | |
| P-23 | N-(4-(4-(4-(3-amino-6-(2- | m/z (ESI⁺) 576 (M + H)⁺. |
| | hydroxyphenyl)pyridazin-4-yl)- | |
| | 1,4-diazepan-1-yl)pyrimidin-2- | |
| | yl)but-3-yn-1-yl)pyrazolo[1,5- | |
| | a]pyrimidine-2-carboxamide | |
| P-24 | N-(3-(4-(4-(3-amino-6-(2- | ¹H NMR (400 MHz, d₆-DMSO) δ 14.26- |
| | hydroxyphenyl)pyridazin-4- | 14.18 (m, 1H), 9.17 (t, J = 6.0 Hz, 1H), 8.66- |
| | yl)-1,4-diazepan-1-yl) | 8.62 (m, 1H), 8.15-8.09 (m, 1H), 7.88-7.80 |
| | pyrimidin-2-yl)prop-2-yn-1- | (m, 1H), 7.51 (d, J = 7.2 Hz, 1H), 7.25-7.15 |
| | yl)-N-methylpyrazolo[1,5- | (m, 2H), 7.03 (s, 1H), 6.87 (d, J = 8.0 Hz, 2H), |
| | a]pyrimidine-2-carboxamide | 6.77-6.72 (m, 1H), 6.17 (s, 2H), 5.76 (s, 1H), |
| | | 4.90 (s, 1H), 4.62 (s, 1H), 4.07-3.98 (m, 2H), |
| | | 3.72-3.61 (m, 2H), 3.48-3.42 (m, 3H), 3.26- |
| | | 3.25 (m, 1H), 3.14 (s, 1H), 2.10-1.98 (m, 2H), |
| | | 1.23 (s, 2H). |
| P-25 | N-(3-(4-((1R,4R)-5-(3-amino-6- | ¹H NMR (499 MHz, d₆-DMSO) δ 9.17-9.06 |
| | (2-hydroxyphenyl)pyridazin-4-yl)- | (m, 2H), 8.65 (td, J = 4.4, 1.8 Hz, 1H), 8.06 (t, |
| | 2,5-diazabicyclo[2.2.1]heptan-2- | J = 7.4 Hz, 1H), 7.97 (dd, J = 14.7, 7.6 Hz, |
| | yl)pyrimidin-2-yl)prop-2-yn-1- | 1H), 7.26-7.16 (m, 3H), 6.85 (dd, J = 8.1, 1.2 |
| | yl)pyrazolo[1,5-a]pyrimidine-2- | Hz, 1H), 6.38 (d, J = 6.2 Hz, 1H), 5.91 (s, 2H), |
| | carboxamide | 4.84 (s, 1H), 4.33 (d, J = 5.9 Hz, 1H), 4.26 (d, |
| | | J = 5.8 Hz, 1H), 4.06 (dd, J = 5.9, 2.5 Hz, 1H), |
| | | 3.91 (d, J = 9.4 Hz, 1H), 3.53 (dt, J = 19.5, |
| | | 10.4 Hz, 2H), 3.17 (s, 3H), 2.03 (d, J = 13.1 |
| | | Hz, 2H), 1.23 (s, 1H). |
| P-26 | 2-(6-amino-5-(4-(2-(3- | ¹H NMR (499 MHz, d₆-DMSO) δ 14.23 (s, |
| | (benzo[d]oxazol-2-ylamino) | 1H), 8.51 (t, J = 5.9 Hz, 1H), 8.10 (d, J = 6.3 |
| | prop-1-yn-1-yl)pyrimidin-4-yl)- | Hz, 1H), 7.84 (s, 1H), 7.50 (s, 1H), 7.42-7.37 |
| | 1,4-diazepan-1-yl)pyridazin- | (m, 1H), 7.31 (dd, J = 7.8, 1.2 Hz, 1H), 7.21 |
| | 3-yl)phenol | (td, J = 7.6, 1.6 Hz, 1H), 7.15 (td, J = 7.7, 1.2 |
| | | Hz, 1H), 7.03 (td, J = 7.7, 1.3 Hz, 1H), 6.86 (t, |
| | | J = 7.5 Hz, 2H), 6.72 (d, J = 6.3 Hz, 1H), 6.17 |
| | | (s, 2H), 4.39 (d, J = 5.9 Hz, 2H), 4.02 (s, 1H), |
| | | 3.86 (s, 1H), 3.75 (s, 1H), 3.65 (s, 1H), 3.42 (s, |
| | | 2H), 3.25 (t, J = 5.6 Hz, 2H), 2.04 (m, 2H). |
| P-27 | N-(3-(4-(4-(3-amino-6-(2- | m/z (ESI⁺) 576 (M + H)⁺. |
| | hydroxyphenyl)pyridazin-4-yl)- | |
| | 6-methyl-1,4-diazepan-1-yl) | |
| | pyrimidin-2-yl)prop-2-yn-1-yl) | |
| | pyrazolo[1,5-a]pyrimidine-2- | |
| | carboxamide | |
| P-28 | N-(3-(4-(5-(3-amino-6-(2- | m/z (ESI⁺) 560 (M + H)⁺. |
| | hydroxyphenyl)pyridazin-4-yl)- | |
| | 2,5-diazabicyclo[4.1.0]heptan- | |
| | 2-yl)pyrimidin-2-yl)prop-2-yn- | |
| | 1-yl)pyrazolo[1,5-a] | |
| | pyrimidine-2-carboxamide | |
| P-29 | N-(3-(4-(4-(3-amino-6-(2- | m/z (ESI⁺) 576 (M + H)⁺. |
| | hydroxyphenyl)pyridazin-4-yl)- | |
| | 3-methyl-1,4-diazepan-1-yl) | |
| | pyrimidin-2-yl)prop-2-yn-1-yl) | |
| | pyrazolo[1,5-a]pyrimidine- | |
| | 2-carboxamide | |
| P-30 | N-(3-(4-((1S,4S)-5-(3-amino- | m/z (ESI⁺) 560 (M + H)⁺. |
| | 6-(2-hydroxyphenyl)pyridazin- | |
| | 4-yl)-2,5-diazabicyclo[2.2.1] | |
| | heptan-2-yl)pyrimidin-2-yl) | |
| | prop-2-yn-1-yl)pyrazolo[1,5- | |
| | a]pyrimidine-2-carboxamide | |
| P-31 | N-(3-(4-(2-(3-amino-6-(2- | m/z (ESI⁺) 588 (M + H)⁺. |
| | hydroxyphenyl)pyridazin-4-yl)- | |
| | 2,7-diazaspiro[3.5]nonan-7-yl) | |
| | pyrimidin-2-yl)prop-2-yn-1-yl) | |
| | pyrazolo[1,5-a]pyrimidine-2- | |
| | carboxamide | |
| P-32 | N-(3-(4-(8-(3-amino-6-(2- | m/z (ESI⁺) 603 (M + H)⁺. |
| | hydroxyphenyl)pyridazin-4-yl)- | |
| | 2,8-diazaspiro[4.5]decan-2-yl) | |
| | pyrimidin-2-yl)prop-2-yn-1-yl) | |
| | pyrazolo[1,5-a]pyrimidine-2- | |
| | carboxamide | |
| P-33 | N-(3-(4-(2-(3-amino-6-(2- | m/z (ESI⁺) 616 (M + H)⁺. |
| | hydroxyphenyl)pyridazin-4-yl)- | |
| | 2,9-diazaspiro[5.5]undecan-9- | |
| | yl)pyrimidin-2-yl)prop-2-yn-1- | |
| | yl)pyrazolo[1,5-a]pyrimidine- | |
| | 2-carboxamide | |
| P-34 | N-(3-(4-(2-(3-amino-6-(2- | m/z (ESI⁺) 602 (M + H)⁺. |
| | hydroxyphenyl)pyridazin-4-yl)- | |
| | 2,8-diazaspiro[4.5]decan-8-yl) | |
| | pyrimidin-2-yl)prop-2-yn-1-yl) | |
| | pyrazolo[1,5-a]pyrimidine-2- | |
| | carboxamide | |
| P-35 | N-(3-(4-(9-(3-amino-6-(2- | m/z (ESI⁺) 616 (M + H)⁺. |
| | hydroxyphenyl)pyridazin-4-yl)- | |
| | 2,9-diazaspiro[5.5]undecan-2- | |
| | yl)pyrimidin-2-yl)prop-2-yn-1- | |
| | yl)pyrazolo[1,5-a]pyrimidine-2- | |
| | carboxamide | |
| P-36 | N-(3-(4-(5-(3-amino-6-(2- | m/z (ESI⁺) 574 (M + H)⁺. |
| | hydroxyphenyl)pyridazin-4- | |
| | yl)hexahydropyrrolo[3,4- | |
| | b]pyrrol-1(2H)-yl)pyrimidin-2- | |
| | yl)prop-2-yn-1-yl)pyrazolo | |
| | [1,5-a]pyrimidine-2- | |
| | carboxamide | |
| P-37 | N-(3-(4-(6-(3-amino-6-(2- | m/z (ESI⁺) 588 (M + H)⁺. |
| | hydroxyphenyl)pyridazin-4- | |
| | yl)octahydro-1H-pyrrolo[2,3- | |
| | c]pyridin-1-yl)pyrimidin-2- | |
| | y1)prop-2-yn-1-yl)pyrazolo | |
| | [1,5-a]pyrimidine-2- | |
| | carboxamide | |
| P-38 | N-(3-(4-(1-(3-amino-6-(2- | m/z (ESI⁺) 588 (M + H)⁺. |
| | hydroxyphenyl)pyridazin-4- | |
| | yl)octahydro-4H-pyrrolo[3,2- | |
| | b]pyridin-4-yl)pyrimidin-2- | |
| | yl)prop-2-yn-1-yl)pyrazolo | |
| | [1,5-a]pyrimidine-2- | |
| | carboxamide | |
| P-39 | 2-(6-amino-5-(4-(2-(3- | ¹H NMR (400 MHz, d₄-MeOD) δ 8.05 (d, J = |
| | aminoprop-1-yn-1-yl) | 6.0 Hz, 1H), 7.47-7.34 (m, 2H), 7.27 (s, 1H), |
| | pyrimidin-4-yl)-6-methyl- | 7.08-6.98 (m, 2H), 6.78 (s, 1H), 4.69-4.26 |
| | 1,4-diazepan-1-yl)pyridazin- | (m, 3H), 4.06 (s, 2H), 3.99-3.72 (m, 3H), 3.66 |
| | 3-yl)phenol | (s, 1H), 3.39-3.35 (m, 1H), 2.56-2.36 (m, |
| | | 1H), 1.18-1.01 (m, 3H). |
| P-40 | 2-(6-amino-5-(4-(2-(3- | ¹H NMR (400 MHz, d₄-MeOD) δ 8.06-8.11 |
| | aminoprop-1-yn-1-yl) | (m, 1 H) 7.13-7.28 (m, 3 H) 6.87 (d, J = 8 Hz, |
| | pyrimidin-4-yl)-2-methyl- | 2 H) 6.85 (d, J = 4 Hz 1 H) 4.62 (t, J = 4 Hz 1 |
| | 1,4-diazepan-1-yl)pyridazin- | H) 4.25-4.29 (m, 1 H) 3.53-3.79 (m, 6 H) 1.76 |
| | 3-yl)phenol | (s, 2 H) 1.315 (d, J = 4 Hz 1 H) 1.155 (d, J = 8 |
| | | Hz, 3 H). |
| P-41 | N-(3-(4-(5-(3-amino-6-(2- | m/z (ESI⁺) 574 (M + H)⁺. |
| | hydroxyphenyl)pyridazin-4- | |
| | yl)hexahydropyrrolo[3,4-c] | |
| | pyrrol-2(1H)-yl)pyrimidin-2- | |
| | yl)prop-2-yn-1-yl)pyrazolo | |
| | [1,5-a]pyrimidine-2- | |
| | carboxamide | |
| P-42 | N-(3-(4-(9-(3-amino-6-(2- | m/z (ESI⁺) 616 (M + H)⁺. |
| | hydroxyphenyl)pyridazin-4- | |
| | yl)-3,9-diazaspiro[5.5]undecan- | |
| | 3-yl)pyrimidin-2-yl)prop-2-yn- | |
| | 1-yl)pyrazolo[1,5-a]pyrimidine- | |
| | 2-carboxamide | |
| P-43 | N-(3-(4-(3-(3-amino-6-(2- | m/z (ESI⁺) 560 (M + H)⁺. |
| | hydroxyphenyl)pyridazin-4- | |
| | yl)-3,6-diazabicyclo[3.2.0] | |
| | heptan-6-yl)pyrimidin-2-yl) | |
| | prop-2-yn-1-yl)pyrazolo | |
| | [1,5-a]pyrimidine-2- | |
| | carboxamide | |
| P-44 | N-(3-(4-(4-(3-amino-6-(2- | m/z (ESI⁺) 580 (M + H)⁺. |
| | hydroxyphenyl)pyridazin-4- | |
| | yl)-6-fluoro-1,4-diazepan-1- | |
| | yl)pyrimidin-2-yl)prop-2-yn- | |
| | 1-yl)pyrazolo[1,5-a] | |
| | pyrimidine-2-carboxamide | |
| P-45 | N-(3-(4-(6-(3-amino-6-(2- | m/z (ESI⁺) 560 (M + H)⁺. |
| | hydroxyphenyl)pyridazin-4- | |
| | yl)-3,6-diazabicyclo[3.2.0] | |
| | heptan-3-yl)pyrimidin-2-yl) | |
| | prop-2-yn-1-yl)pyrazolo | |
| | [1,5-a]pyrimidine-2- | |
| | carboxamide | |
| P-46 | N-(3-(4-(4-(3-amino-6-(2- | m/z (ESI⁺) 576 (M + H)⁺. |
| | hydroxyphenyl)pyridazin- | |
| | 4-yl)-7-methyl-1,4-diazepan- | |
| | 1-yl)pyrimidin-2-yl)prop-2- | |
| | yn-1-yl)pyrazolo[1,5-a] | |
| | pyrimidine-2-carboxamide | |
| P-47 | N-(3-(4-((1R,5R)-2-(3- | m/z (ESI⁺) 560 (M + H)⁺. |
| | amino-6-(2-hydroxyphenyl) | |
| | pyridazin-4-yl)-2,6- | |
| | diazabicyclo[3.2.0]heptan- | |
| | 6-yl)pyrimidin-2-yl)prop- | |
| | 2-yn-1-yl)pyrazolo[1,5-a] | |
| | pyrimidine-2-carboxamide | |
| P-48 | (S)-N-(3-(4-(4-(3-amino- | m/z (ESI⁺) 562 (M + H)⁺. |
| | 6-(2-hydroxyphenyl) | |
| | pyridazin-4-yl)-3- | |
| | methylpiperazin-1-yl) | |
| | pyrimidin-2-yl)prop-2- | |
| | yn-1-yl)pyrazolo[1,5-a] | |
| | pyrimidine-2-carboxamide | |
| P-49 | (R)-N-(3-(4-(4-(3-amino- | m/z (ESI⁺) 562 (M + H)⁺. |
| | 6-(2-hydroxyphenyl) | |
| | pyridazin-4-yl)-3- | |
| | methylpiperazin-1-yl) | |
| | pyrimidin-2-yl)prop-2-yn- | |
| | 1-yl)pyrazolo[1,5-a] | |
| | pyrimidine-2-carboxamide | |
| P-50 | (R)-N-(3-(4-(4-(3-amino- | m/z (ESI⁺) 562 (M + H)⁺. |
| | 6-(2-hydroxyphenyl) | |
| | pyridazin-4-yl)-2- | |
| | methylpiperazin-1-yl) | |
| | pyrimidin-2-yl)prop-2- | |
| | yn-1-yl)pyrazolo[1,5-a] | |
| | pyrimidine-2-carboxamide | |
| P-51 | N-(3-(4-(4-(3-amino-6-(2- | m/z (ESI⁺) 576 (M + H)⁺. |
| | hydroxyphenyl)pyridazin- | |
| | 4-yl)-3-ethylpiperazin-1- | |
| | yl)pyrimidin-2-yl)prop-2- | |
| | yn-1-yl)pyrazolo[1,5-a] | |
| | pyrimidine-2-carboxamide | |
| P-52 | N-(3-(4-(4-(3-amino-6-(2- | m/z (ESI⁺) 565 (M + H)⁺. |
| | hydroxyphenyl)pyridazin-4- | |
| | yl)piperazin-1-yl)pyrimidin- | |
| | 2-yl)prop-2-yn-1-yl)-6- | |
| | fluorobenzofuran-2- | |
| | carboxamide | |
| P-53 | N-(3-(4-(4-(3-amino-6-(2- | m/z (ESI⁺) 562 (M + H)⁺. |
| | hydroxyphenyl)pyridazin-4- | |
| | yl)piperazin-1-yl)pyrimidin- | |
| | 2-yl)prop-2-yn-1-yl)-6- | |
| | fluorobenzofuran-2- | |
| | carboxamide | |
| P-54 | N-(3-(4-(4-(3-amino-6-(2- | m/z (ESI⁺) 548 (M + H)⁺. |
| | hydroxyphenyl)pyridazin-4- | |
| | yl)piperazin-1-yl)pyrimidin- | |
| | 2-yl)prop-2-yn-1-yl)benzo | |
| | [d]isoxazole-3-carboxamide | |
| P-55 | N-(3-(4-(4-(3-amino-6-(2- | m/z (ESI⁺) 547 (M + H)⁺. |
| | hydroxyphenyl)pyridazin-4- | |
| | yl)piperazin-1-yl)pyrimidin-2- | |
| | yl)prop-2-yn-1-yl)benzofuran- | |
| | 3-carboxamide | |
| P-56 | N-(3-(4-(4-(3-amino-6-(2- | m/z (ESI⁺) 508 (M + H)⁺. |
| | hydroxyphenyl)pyridazin-4- | |
| | yl)piperazin-1-yl)pyrimidin- | |
| | 2-yl)prop-2-yn-1-yl) | |
| | isonicotinamide | |
| P-57 | N-(3-(4-(4-(3-amino-6-(2- | m/z (ESI⁺) 553 (M + H)⁺. |
| | hydroxyphenyl)pyridazin-4- | |
| | yl)piperazin-1-y1)pyrimidin- | |
| | 2-yl)prop-2-yn-1-yl)-6,7- | |
| | dihydro-5H-pyrazolo[5,1-b] | |
| | [1,3]oxazine-2-carboxamide | |
| P-58 | N-(3-(4-(4-(3-amino-6-(2- | m/z (ESI⁺) 553 (M + H)⁺. |
| | hydroxyphenyl)pyridazin-4- | |
| | yl)piperazin-1-yl)pyrimidin-2- | |
| | yl)prop-2-yn-1-yl)-6,7-dihydro- | |
| | 4H-pyrazolo[5,1-c][1,4] | |
| | oxazine-2-carboxamide | |
| P-59 | N-(3-(4-(4-(3-amino-6-(2- | m/z (ESI⁺) 537 (M + H)⁺. |
| | hydroxyphenyl)pyridazin-4- | |
| | yl)piperazin-1-yl)pyrimidin-2- | |
| | yl)prop-2-yn-1-yl)-5,6-dihydro- | |
| | 4H-pyrrolo[1,2-b]pyrazole-2- | |
| | carboxamide | |
| P-60 | N-(3-(4-(4-(3-amino-6-(2- | m/z (ESI⁺) 548 (M + H)⁺. |
| | hydroxyphenyl)pyridazin-4- | |
| | yl)piperazin-1-yl)pyrimidin-2- | |
| | yl)prop-2-yn-1-yl)imidazo[1,2- | |
| | a]pyrazinc-2-carboxamidc | |
| P-61 | N-(3-(4-(4-(3-amino-6-(2- | m/z (ESI⁺) 565 (M + H)⁺. |
| | hydroxyphenyl)pyridazin-4- | |
| | yl)piperazin-1-yl)pyrimidin-2- | |
| | yl)prop-2-yn-1-yl)-2,3- | |
| | dihydrobenzo[b][1,4]dioxine- | |
| | 2-carboxamide | |
| P-62 | N-(3-(4-(4-(3-amino-6-(2- | m/z (ESI⁺) 528 (M + H)⁺. |
| | hydroxyphenyl)pyridazin-4- | |
| | yl)piperazin-1-yl)pyrimidin- | |
| | 2-yl)prop-2-yn-1-yl)-4- | |
| | methylthiazole-5-carboxamide | |
| P-63 | N-(3-(4-(4-(3-amino-6-(2- | m/z (ESI⁺) 547 (M + H)⁺. |
| | hydroxyphenyl)pyridazin-4- | |
| | yl)piperazin-1-yl)pyrimidin-2- | |
| | yl)prop-2-yn-1-yl)benzofuran-7- | |
| | carboxamide | |
| P-64 | N-(3-(4-(4-(3-amino-6-(2- | m/z (ESI⁺) 558 (M + H)⁺. |
| | hydroxyphenyl)pyridazin-4- | |
| | yl)piperazin-1-yl)pyrimidin-2- | |
| | yl)prop-2-yn-1-yl)quinoline-2- | |
| | carboxamide | |
| P-65 | N-(3-(4-(4-(3-amino-6-(2- | m/z (ESI⁺) 547 (M + H)⁺. |
| | hydroxyphenyl)pyridazin-4- | |
| | yl)piperazin-1-yl)pyrimidin-2- | |
| | yl)prop-2-yn-1-yl)pyrazolo[1, | |
| | 5-a]pyridine-3-carboxamide | |
| P-66 | N-(3-(4-(4-(3-amino-6-(2- | m/z (ESI⁺) 547 (M + H)⁺. |
| | hydroxyphenyl)pyridazin-4- | |
| | yl)piperazin-1-yl)pyrimidin-2- | |
| | yl)prop-2-yn-1-yl)imidazo[1,2- | |
| | a]pyridine-3-carboxamide | |
| P-67 | N-(3-(4-(4-(3-amino-6-(2- | m/z (ESI⁺) 511 (M + H)⁺. |
| | hydroxyphenyl)pyridazin-4- | |
| | yl)piperazin-1-yl)pyrimidin-2- | |
| | yl)prop-2-yn-1-yl)-1-methyl- | |
| | 1H-imidazole-4-carboxamide | |
| P-68 | N-(3-(4-(4-(3-amino-6-(2- | m/z (ESI⁺) 445 (M + H)⁺. |
| | hydroxyphenyl)pyridazin-4- | |
| | yl)piperazin-1-yl)pyrimidin-2- | |
| | yl)prop-2-yn-1-yl)acetamide | |
| P-69 | N-(3-(4-(4-(3-amino-6-(2- | m/z (ESI⁺) 551 (M + H)⁺. |
| | hydroxyphenyl)pyridazin-4- | |
| | yl)piperazin-1-yl)pyrimidin-2- | |
| | yl)prop-2-yn-1-yl)-4,5,6,7- | |
| | tetrahydropyrazolo[1,5-a] | |
| | pyridine-2-carboxamide | |
| P-70 | N-(3-(4-(4-(3-amino-6-(2- | m/z (ESI⁺) 576 (M + H)⁺. |
| | hydroxyphenyl)pyridazin-4- | |
| | yl)piperazin-1-yl)pyrimidin- | |
| | 2-yl)prop-2-yn-1-yl)-5,7- | |
| | dimethylpyrazolo[1,5-a] | |
| | pyrimidine-3-carboxamide | |
| P-71 | N-(3-(4-(4-(3-amino-6-(2- | ¹H NMR (499 MHz, d₆-DMSO) δ 9.33 (dd, J = |
| | hydroxyphenyl)pyridazin-4- | 7.0, 1.7 Hz, 1H), 8.85 (dd, J = 4.2, 1.7 Hz, 1H), |
| | yl)piperazin-1-yl)pyrimidin-2- | 8.64 (s, 1H), 8.36 (t, J = 5.8 Hz, 1H), 8.28 (s, |
| | yl)prop-2-yn-1-yl)pyrazolo | 0H), 8.18 (dd, J = 6.1, 2.8 Hz, 1H), 7.91 (dd, |
| | [1,5-a]pyrimidine-3- | J = 8.1, 1.6 Hz, 1H), 7.55 (s, 1H), 7.33-7.17 |
| | carboxamide | (m, 2H), 6.96-6.88 (m, 1H), 6.88 (t, J = 7.4 |
| | | Hz, 2H), 6.43 (s, 2H), 4.45 (d, J = 5.9 Hz, 2H), |
| | | 3.86 (s, 4H), 3.14 (t, J = 5.1 Hz, 4H), 3.02 (s, |
| | | 1H), 0.97 (d, J = 6.6 Hz, 1H). |
| P-72 | N-(3-(4-(4-(3-amino-6-(2- | m/z (ESI⁺) 559 (M + H)⁺. |
| | hydroxyphenyl)pyridazin-4- | |
| | yl)piperazin-1-yl)pyrimidin- | |
| | 2-yl)prop-2-yn-1-yl) | |
| | quinoxaline-2-carboxamide | |
| P-73 | N-(3-(4-(4-(3-amino-6-(2- | m/z (ESI⁺) 546 (M + H)⁺. |
| | hydroxyphenyl)pyridazin-4- | |
| | yl)piperazin-1-yl)pyrimidin- | |
| | 2-yl)prop-2-yn-1-yl)-1H- | |
| | indole-2-carboxamide | |
| P-74 | N-(3-(4-(4-(3-amino-6-(2- | m/z (ESI⁺) 561 (M + H)⁺. |
| | hydroxyphenyl)pyridazin-4- | |
| | yl)piperazin-1-yl)pyrimidin-2- | |
| | yl)prop-2-yn-1-yl)-3- | |
| | methylbenzofuran-2- | |
| | carboxamide | |
| P-75 | N-(3-(4-(4-(3-amino-6-(2- | m/z (ESI⁺) 547 (M + H)⁺. |
| | hydroxyphenyl)pyridazin-4- | |
| | yl)piperazin-1-yl)pyrimidin-2- | |
| | yl)prop-2-yn-1-yl)benzofuran- | |
| | 2-carboxamide | |
| P-76 | N-(3-(4-(4-(3-amino-6-(2- | ¹H NMR (499 MHz, d₆-DMSO) δ 14.21 (s, |
| | hydroxyphenyl)pyridazin-4-yl)- | 1H), 9.18-9.11 (m, 2H), 8.65 (dd, J = 4.0, 1.7 |
| | 1,4-diazepan-1-yl)pyrimidin- | Hz, 1H), 8.12 (d, J = 6.2 Hz, 1H), 7.84 (s, 1H), |
| | 2-yl)prop-2-yn-1-yl)pyrazolo | 7.51 (s, 1H), 7.25-7.17 (m, 2H), 7.13 (d, J = |
| | [1,5-a]pyrimidine-2- | 0.9 Hz, 1H), 6.86 (t, J = 7.7 Hz, 2H), 6.73 (d, |
| | carboxamide | J = 6.3 Hz, 1H), 6.16 (s, 2H), 4.33 (d, J = 5.9 |
| | | Hz, 2H), 4.03 (q, J = 7.1 Hz, 1H), 3.88 (s, 1H), |
| | | 3.76 (s, 1H), 3.66 (s, 1H), 3.42 (d, J = 5.3 Hz, |
| | | 2H), 3.25 (t, J = 5.6 Hz, 2H), 1.99 (s, 1H), 1.17 |
| | | (t, J = 7.1 Hz, 1H). |
| P-77 | N-(3-(4-(4-(3-amino-6-(2- | m/z (ESI⁺) 574 (M + H)⁺. |
| | hydroxyphenyl)pyridazin-4- | |
| | yl)hexahydropyrrolo[3,2-b] | |
| | pyrrol-1(2H)-yl)pyrimidin-2- | |
| | yl)prop-2-yn-1-yl)pyrazolo | |
| | [1,5-a]pyrimidine-2- | |
| | carboxamide | |
| P-78 | N-(3-(4-(8-(3-amino-6-(2- | ¹H NMR (499 MHz, d₆-DMSO) δ 9.18-9.12 |
| | hydroxyphenyl)pyridazin-4-yl)- | (m, 2H), 8.66 (dd, J = 4.0, 1.7 Hz, 1H), 8.17 |
| | 3,8-diazabicyclo[3.2.1]octan- | (d,J = 6.2 Hz, 1H), 7.99-7.93 (m, 1H), 7.45 (s, |
| | 3-yl)pyrimidin-2-yl)prop- | 1H), 7.27-7.18 (m, 3H), 7.13 (d, J = 0.9 Hz, |
| | y2-yn-1-l)pyrazolo[1,5-a] | 1H), 6.89 (ddd, J = 8.1, 6.7, 1.3 Hz, 2H), 6.79 |
| | pyrimidine-2-carboxamide | (d, J = 6.4 Hz, 1H), 6.19 (s, 2H), 4.46 (d, J = |
| | | 4.6 Hz, 2H), 4.33 (d, J = 5.9 Hz, 2H), 4.05 (s, |
| | | 2H), 2.00-1.94 (m, 2H), 1.67 (q, J = 6.4 Hz, |
| | | 2H). |
| P-79 | N-(3-(4-(4-(3-amino-6-(2- | m/z (ESI⁺) 588 (M + H)⁺. |
| | hydroxyphenyl)pyridazin-4- | |
| | yl)octahydro-1H-pyrrolo[3,2- | |
| | b]pyridin-1-yl)pyrimidin-2-yl) | |
| | prop-2-yn-1-yl)pyrazolo[1,5-a] | |
| | pyrimidine-2-carboxamide | |
| P-80 | N-(3-(4-(6-(3-amino-6-(2- | m/z (ESI⁺) 560 (M + H)⁺. |
| | hydroxyphenyl)pyridazin-4-yl)- | |
| | 3,6-diazabicyclo[3.1.1]heptan- | |
| | 3-yl)pyrimidin-2-yl)prop- | |
| | 2-yn-1-yl)pyrazolo[1,5-a] | |
| | pyrimidine-2-carboxamide | |
| P-81 | (R)-N-(3-(4-(4-(3-amino-6- | m/z (ESI⁺) 562 (M + H)⁺. |
| | (2-hydroxyphenyl)pyridazin- | |
| | 4-yl)-2-methylpiperazin-1- | |
| | yl)pyrimidin-2-yl)prop-2- | |
| | yn-1-yl)pyrazolo[1,5-a] | |
| | pyrimidine-2-carboxamide | |
| P-82 | N-(3-(4-((3R,5R)-4-(3- | m/z (ESI⁺) 576 (M + H)⁺. |
| | amino-6-(2-hydroxyphenyl) | |
| | pyridazin-4-yl)-3,5- | |
| | dimethylpiperazin-1-yl) | |
| | pyrimidin-2-yl)prop- | |
| | 2-yn-1-yl)pyrazolo[1,5- | |
| | a]pyrimidine-2-carboxamide | |
| P-83 | N-(3-(4-(4-(3-amino-6-(2- | m/z (ESI⁺) 576 (M + H)⁺. |
| | hydroxyphenyl)pyridazin-4- | |
| | yl)-3,3-dimethylpiperazin- | |
| | 1-yl)pyrimidin-2-yl)prop- | |
| | 2-yn-1-yl)pyrazolo[1,5- | |
| | a]pyrimidine-2-carboxamide | |
| P-84 | 2-(6-amino-5-(3-(2-(3- | m/z (ESI⁺) 415 (M + H)⁺. |
| | aminoprop-1-yn-1-yl) | |
| | pyrimidin-4-yl)-3,6- | |
| | diazabicyclo[3.2.0]heptan-6- | |
| | yl)pyridazin-3-yl)phenol | |
| P-85 | 2-(6-amino-5-(4-(2-(3- | m/z (ESI⁺) 431 (M + H)⁺. |
| | aminoprop-1-yn-1-yl) | |
| | pyrimidin-4-yl)-5-methyl- | |
| | 1,4-diazepan-1-yl)pyridazin- | |
| | 3-yl)phenol | |
| P-86 | (S)-2-(6-amino-5-(4-(2-(3- | m/z (ESI⁺) 417 (M + H)⁺. |
| | aminoprop-1-yn-1-yl) | |
| | pyrimidin-4-yl)-2- | |
| | methylpiperazin-1-yl) | |
| | pyridazin-3-yl)phenol | |
| P-87 | 2-(6-amino-5-(2-(2-(3- | m/z (ESI⁺) 472 (M + H)⁺. |
| | aminoprop-1-yn-1-yl) | |
| | pyrimidin-4-yl)-2,9- | |
| | diazaspiro[5.5]undecan-9- | |
| | yl)pyridazin-3-yl)phenol | |
| P-88 | 2-(6-amino-5-(1-(2-(3- | m/z (ESI⁺) 443 (M + H)⁺. |
| | aminoprop-1-yn-1-yl) | |
| | pyrimidin-4-yl)octahydro- | |
| | 4H-pyrrolo[3,2-b]pyridin-4- | |
| | yl)pyridazin-3-yl)phenol | |
| P-89 | 2-(6-amino-5-(9-(2-(3- | m/z (ESI⁺) 472 (M + H)⁺. |
| | aminoprop-1-yn-1-yl) | |
| | pyrimidin-4-yl)-2,9- | |
| | diazaspiro[5.5]undecan-2- | |
| | yl)pyridazin-3-yl)phenol | |
| P-90 | 2-(6-amino-5-(9-(2-(3- | m/z (ESI⁺) 472 (M + H)⁺. |
| | aminoprop-1-yn-1-yl) | |
| | pyrimidin-4-yl)-3,9- | |
| | diazaspiro[5.5]undecan-3- | |
| | yl)pyridazin-3-yl)phenol | |
| P-91 | 2-(6-amino-5-(6-(2-(3- | m/z (ESI⁺) 415 (M + H)⁺. |
| | aminoprop-1-yn-1-yl) | |
| | pyrimidin-4-yl)-3,6- | |
| | diazabicyclo[3.1.1]heptan- | |
| | 3-yl)pyridazin-3-yl) | |
| | phenol | |
| P-92 | N-(5-(4-(4-(3-amino-6-(2- | m/z (ESI⁺) 577 (M + H)⁺. |
| | hydroxyphenyl)pyridazin-4- | |
| | yl)piperazin-1-yl)pyrimidin- | |
| | 2-yl)pent-4-yn-1-yl)pyrazolo | |
| | [1,5-a]pyrimidinc-2- | |
| | carboxamidc | |
| P-93 | N-(3-(4-(4-(3-amino-6-(2- | ¹H NMR (499 MHz, d₆-DMSO) δ 14.16 (s, |
| | hydroxyphenyl)pyridazin-4- | 1H), 9.19-9.12 (m, 2H), 8.66 (dd, J = 4.0, 1.7 |
| | yl)piperazin-1-yl)pyrimidin- | Hz, 1H), 8.18 (d, J = 6.3 Hz, 1H), 7.91 (dd, J = |
| | 2-yl)prop-2-yn-1-yl)pyrazolo | 8.0, 1.6 Hz, 1H), 7.55 (s, 1H), 7.27-7.17 (m, |
| | [1,5-a]pyrimidine-2- | 2H), 7.13 (d, J = 0.9 Hz, 1H), 6.93 6.84 (m, |
| | carboxamide | 3H), 6.44 (s, 2H), 4.33 (d, J = 5.9 Hz., 2H), |
| | | 3.86 (s, 4H), 3.14 (t, J = 5.0 Hz, 4H). |
| P-94 | N-(4-(4-(4-(3-amino-6-(2- | m/z (ESI⁺) 563 (M + H)⁺. |
| | hydroxyphenyl)pyridazin-4- | |
| | yl)piperazin-1-yl)pyrimidin- | |
| | 2-yl)but-3-yn-1-yl)pyrazolo | |
| | [1,5-a]pyrimidine-2- | |
| | carboxamide | |
| P-95 | N-(4-(4-(4-(3-amino-6-(2- | m/z (ESI⁺) 563 (M + H)⁺. |
| | hydroxyphenyl)pyridazin-4- | |
| | yl)piperazin-1-yl)pyrimidin- | |
| | 2-yl)but-3-yn-2-yl)pyrazolo | |
| | [1,5-a]pyrimidine-2- | |
| | carboxamide | |
| P-96 | N-(4-(4-(3-(3-amino-6-(2- | m/z (ESI⁺) 589 (M + H)⁺. |
| | hydroxyphenyl)pyridazin- | |
| | 4-yl)-3,8-diazabicyclo | |
| | [3.2.1]octan-8-yl) | |
| | pyrimidin-2-yl)but-3-yn- | |
| | 1-yl)pyrazolo[1,5-a] | |
| | pyrimidine-2-carboxamide | |
| P-97 | N-(4-(4-(3-(3-amino-6-(2- | m/z (ESI⁺) 589 (M + H)⁺. |
| | hydroxyphenyl)pyridazin- | |
| | 4-yl)-3,8-diazabicyclo | |
| | [3.2.1]octan-8-yl) | |
| | pyrimidin-2-yl)but-3- | |
| | yn-2-yl)pyrazolo\|1,5-a\| | |
| | pyrimidine-2-carboxamide | |
| P-98 | N-(1-((4-(3-(3-amino-6-(2- | m/z (ESI⁺) 601 (M + H)⁺. |
| | hydroxyphenyl)pyridazin-4- | |
| | yl)-3,8-diazabicyclo[3.2.1] | |
| | octan-8-yl)pyrimidin-2- | |
| | yl)ethynyl)cyclopropyl) | |
| | pyrazolo[1,5-a] | |
| | pyrimidine-2-carboxamide | |
| P-99 | N-(5-(4-(3-(3-amino-6-(2- | m/z (ESI⁺) 603 (M + H)⁺. |
| | hydroxyphenyl)pyridazin- | |
| | 4-yl)-3,8-diazabicyclo | |
| | [3.2.1]octan-8-yl) | |
| | pyrimidin-2-yl)pent-4- | |
| | yn-1-yl)pyrazolo[1,5-a] | |
| | pyrimidine-2-carboxamide | |
| P-100 | 2-(6-amino-5-(8-(2-(3- | m/z (ESI⁺) 546 (M + H)⁺. |
| | (benzo[d]doxazol-2-ylamino) | |
| | prop-1-yn-1-yl) | |
| | pyrimidin-4-yl)-3,8- | |
| | diazabicyclo[3.2.1]octan-3- | |
| | yl)pyridazin-3-yl)phenol | |
| P-101 | (S)-N-(3-(4-(4-(3-amino- | m/z (ESI⁺) 587 (M + H)⁺. |
| | 6-(2-hydroxyphenyl) | |
| | pyridazin-4-yl)-7-methyl- | |
| | 1,4-diazepan-1-yl)pyrimidin- | |
| | 2-y1)prop-2-yn-1-yl) | |
| | quinoxaline-2-carboxamide | |
| P-102 | (S)-N-(3-(4-(4-(3-amino-6- | m/z (ESI⁺) 576 (M + H)⁺. |
| | (2-hydroxyphenyl)pyridazin- | |
| | 4-yl)-7-methyl-1,4- | |
| | diazepan-1-yl)pyrimidin-2- | |
| | yl)prop-2-yn-1-yl)pyrazolo | |
| | [1,5-a]pyrimidine- | |
| | 2-carboxamide | |
| P-103 | 5-(3-(4-(3-(3-amino-6-(2- | m/z (ESI⁺) 575 (M + H)⁺. |
| | hydroxyphenyl)pyridazin- | |
| | 4-yl)-3,8-diazabicyclo | |
| | [3.2.1]octan-8-yl) | |
| | pyrimidin-2-yl)prop-2- | |
| | yn-1-yl)-2,3- | |
| | dimethylpyrazolo[1,5-a] | |
| | pyrazin-4(5H)-one | |
| P-104 | 2-(6-amino-5-(4-(2-(3- | m/z (ESI⁺) 552 (M + H)⁺. |
| | (3,5-diisopropyl-1H- | |
| | pyrazol-1-yl)prop-1- | |
| | yn-1-yl)pyrimidin-4-yl)- | |
| | 1,4-diazepan-1-yl)pyridazin- | |
| | 3-yl)phenol | |
| P-105 | tert-butyl 2-(3-(4-(4-(3-amino- | m/z (ESI⁺) 610 (M + H)⁺. |
| | 6-(2-hydroxyphenyl) | |
| | pyridazin-4-yl)-1,4- | |
| | diazepan-1-yl)pyrimidin-2-yl) | |
| | prop-2-yn-1-yl)-2,6- | |
| | dihydropyrrolo[3,4- | |
| | c]pyrazole-5(4H)-carboxylate | |
| P-106 | 2-(6-amino-5-(8-(2-(3-(oxazol-2- | m/z (ESI⁺) 496 (M + H)⁺. |
| | ylamino)prop-1-yn-1-yl)pyrimidin-4- | |
| | yl)-3,8-diazabicyclo[3.2.1]octan-3- | |
| | yl)pyridazin-3-yl)phenol | |
| P-107 | 2-(6-amino-5-(4-(2-(3- | ¹H NMR (400 MHz, d₄-methanol) δ 8.03 (br s, |
| | (benzo[d]oxazol-2- | 1H), 7.64 (br d, J = 7.4 Hz, 1H), 7.47 (br s, |
| | yl(methyl)amino)prop-1-yn-1- | 1H), 7.36 (br dd, J = 7.6, 17.6 Hz, 2H), 7.21 |
| | yl)pyrimidin-4-yl)-1,4-diazepan-1- | (br d, J = 5.4 Hz, 2H), 7.10 (br d, J = 7.3 Hz, |
| | yl)pyridazin-3-yl)phenol | 1H), 6.90 (br d, J = 7.0 Hz, 2H), 6.78-6.60 |
| | | (m, 1H), 4.95-4.92 (m, 2H), 4.66 (br s, 2H), |
| | | 4.30-4.08 (m, 1H), 3.92-3.67 (m, 2H), 3.53 |
| | | (br s, 2H), 3.46-3.35 (m, 5H), 2.13 (br s, 2H). |
| P-108 | 2-(6-amino-5-(4-(2-(3-((1-methyl-1H- | ¹H NMR (400 MHz, d₄-methanol) δ 8.05 (d, J |
| | pyrazol-3-yl)oxy)prop-1-yn-1- | = 6.1 Hz, 1H), 7.65 (dd, J = 1.3, 8.2 Hz, 1H), |
| | yl)pyrimidin-4-yl)-1,4-diazepan-1- | 7.50 (s, 1H), 7.39 (d, J = 2.4 Hz, 1H), 7.33- |
| | yl)pyridazin-3-yl)phenol | 7.19 (m, 1H), 7.01-6.85 (m, 2H), 6.78-6.65 |
| | | (m, 1H), 5.78 (d, J = 2.4 Hz, 1H), 5.01 (s, 2H), |
| | | 4.60 (br s, 1H), 4.26-4.11 (m, 1H), 3.99-3.78 |
| | | (m, 2H), 3.75 (s, 3H), 3.62 (br s, 2H), 3.44 (d, |
| | | J = 11.6 Hz, 2H), 2.16 (br s, 2H). |
| P-109 | 5-(3-(4-(4-(3-amino-6-(2- | ¹H NMR (400 MHz, d₄-methanol) δ 8.05 (d, J |
| | hydroxyphenyl)pyridazin-4-yl)-1,4- | = 6.1 Hz, 1H), 7.91 (d, J = 2.0 Hz, 1H), 7.76 |
| | diazepan-1-yl)pyrimidin-2-yl)prop-2- | (d, J = 6.0 Hz, 1H), 7.70-7.62 (m, 1H), 7.49 |
| | yn-1-yl)pyrazolo[1,5-a]pyrazin- | (s, 1H), 7.29-7.19 (m, 2H), 7.13 (d, J = 1.6 |
| | 4(5H)-one | Hz, 1H), 6.96-6.86 (m, 2H), 6.71 (d, J = 5.4 |
| | | Hz, 1H), 5.07 (s, 2H), 4.60 (s, 1H), 4.26-4.08 |
| | | (m, 1H), 3.88-3.70 (m, 2H), 3.57 (s, 2H), 3.41 |
| | | (br s, 3H), 2.15 (br s, 2H). |
| P-110 | (E)-N-(3-(4-(4-(3-amino-6-(2- | m/z (ESI⁺) 575 (M + H)⁺. |
| | hydroxyphenyl)pyridazin-4-yl)-1,4- | |
| | diazepan-1-yl)pyrimidin-2- | |
| | yl)allyl)quinoxaline-2-carboxamide | |
| P-111 | (E)-N-(3-(4-(4-(3-amino-6-(2- | m/z (ESI⁺) 555 (M + H)⁺. |
| | hydroxyphenyl)pyridazin-4-yl)-1,4- | |
| | diazepan-1-yl)pyrimidin-2-yl)allyl)- | |
| | 1,4,5-trimethyl-1H-pyrazole-3- | |
| | carboxamide | |
| P-112 | (E)-N-(3-(4-(4-(3-amino-6-(2- | m/z (ESI⁺) 564 (M + H)⁺. |
| | hydroxyphenyl)pyridazin-4-yl)-1,4- | |
| | diazepan-1-yl)pyrimidin-2- | |
| | yl)allyl)pyrazolo[1,5-a]pyrimidine-2- | |
| | carboxamide | |
| P-113 | (E)-2-(6-amino-5-(4-(2-(3- | m/z (ESI⁺) 419 (M + H)⁺. |
| | aminoprop-1-en-1-yl)pyrimidin-4-yl)- | |
| | 1,4-diazepan-1-yl)pyridazin-3- | |
| | yl)phenol | |
| P-114 | tert-butyl (E)-(3-(4-(4-(3-amino-6-(2- | m/z (ESI⁺) 519 (M + H)⁺. |
| | hydroxyphenyl)pyridazin-4-yl)-1,4- | |
| | diazepan-1-yl)pyrimidin-2- | |
| | yl)allyl)carbamate | |
| P-115 | 5-(3-(4-(4-(3-amino-6-(2- | m/z (ESI⁺) 563 (M + H)⁺. |
| | hydroxyphenyl)pyridazin-4-yl)-1,4- | |
| | diazepan-1-yl)pyrimidin-2-yl)prop-2- | |
| | yn-1-yl)-2,3-dimethylpyrazolo[1,5- | |
| | a]pyrazin-4(5H)-one | |
| P-116 | 2-(5-(4-(2-(3-(1H-pyrazolo[3,4- | m/z (ESI⁺) 519 (M + H)⁺. |
| | c]pyridin-1-yl)prop-1-yn-1- | |
| | yl)pyrimidin-4-yl)-1,4-diazepan-1- | |
| | yl)-6-aminopyridazin-3-yl)phenol | |
| P-117 | N-(3-(4-(4-(3-amino-6-(2- | m/z (ESI⁺) 565 (M + H)⁺. |
| | hydroxyphenyl)pyridazin-4- | |
| | yl)piperazin-1-yl)pyrimidin-2- | |
| | yl)prop-2-yn-1-yl)-4- | |
| | fluoropyrazolo[1,5-a]pyridine-3- | |
| | carboxamide | |
| P-118 | N-(3-(4-(4-(3-amino-6-(2- | m/z (ESI⁺) 564 (M + H)⁺. |
| | hydroxyphenyl)pyridazin-4- | |
| | yl)piperazin-1-yl)pyrimidin-2- | |
| | yl)prop-2-yn-1-yl)benzo[d]thiazole-2- | |
| | carboxamide | |
| P-119 | N-(3-(4-(3-(3-amino-6-(2- | m/z (ESI⁺) 575 (M + H)⁺. |
| | hydroxyphenyl)pyridazin-4-yl)-3,8- | |
| | diazabicyclo[3.2.1]octan-8- | |
| | yl)pyrimidin-2-yl)prop-2-yn-1- | |
| | yl)pyrazolo[1,5-a]pyrimidine-2- | |
| | carboxamide | |
| P-120 | 2-(6-amino-5-(4-(2-(3-(pyrazolo[1,5- | ¹H NMR (400 MHz, CDCl₃) δ 8.58-8.48 (m, |
| | a]pyrimidin-2-ylamino)prop-1-yn-1- | 1H), 8.28 (d, J = 3.6 Hz, 1H), 8.21-8.12 (m, |
| | yl)pyrimidin-4-yl)-1,4-diazepan-1- | 1H), 7.52 (d, J = 7.2 Hz, 1H), 7.32 (s, 1H), |
| | yl)pyridazin-3-yl)phenol | 7.06 (d, J = 8.0 Hz, 2H), 6.93-6.85 (m, 1H), |
| | | 6.62-6.56 (m, 1H), 6.45 (d, J = 6.4 Hz, 1H), |
| | | 6.02 (s, 1H), 4.39 (s, 2H), 4.17-3.96 (m, 2H), |
| | | 3.86-3.65 (m, 2H), 3.46 (t, J = 6.0 Hz, 2H), |
| | | 3.37-3.31 (m, 2H), 2.10 (d, J = 7.2 Hz, 2H), |
| | | 0.93-0.81 (m, 2H). |
| P-121 | tert-butyl (3-(4-(4-(3-amino-6-(2- | ¹H NMR (400 MHz, d₄-methanol) δ 7.97-7.90 |
| | hydroxyphenyl)pyridazin-4-yl)-1,4- | (m, 1H), 7.44-7.37 (m, 1H), 7.27-7.19 (m, |
| | diazepan-1-yl)pyrimidin-2-yl)prop-2- | 2H), 6.90-6.82 (m, 2H), 6.69 (s, 1H), 6.54 (d, |
| | yn-1-yl)carbamate | J = 6.0 Hz, 1H), 4.09 - 3.88 (m, 3H), 3.87 (s, |
| | | 2H), 3.67 (s, 3H), 3.54 - 3.42 (m, 2H), 2.06 - |
| | | 1.96 (m, 2H), 1.28 - 1.18 (m, 9H). |
| P-122 | N-(3-(4-(4-(3-amino-6-(2- | ¹H NMR (400 MHz, d₄-methanol) δ 8.15 (s, |
| | hydroxyphenyl)pyridazin-4-yl)-1,4- | 1H), 7.73 - 7.61 (m, 1H), 7.49 (s, 1H), 7.27 (t, |
| | diazepan-1-yl)pyrimidin-2-yl)prop-2- | J = 7.2 Hz, 1H), 7.04 - 6.87 (m, 2H), 6.70 (d, J |
| | yn-1-yl)acetamide | = 4.4 Hz, 1H), 4.26 - 4.19 (m, 2H), 4.18 - 3.92 |
| | | (m, 2H), 3.91 - 3.71 (m, 2H), 3.60 (s, 2H), 3.43 |
| | | (s, 2H), 2.22 - 2.10 (m, 2H), 2.00 (s, 3H). |
| P-123 | 2-(6-amino-5-(2-(2-(3-hydroxyprop- | m/z (ESI⁺) 472 (M + H)⁺. |
| | 1-yn-1-yl)pyrimidin-4-yl)-2,9- | |
| | diazaspiro[5.5]undecan-9- | |
| | yl)pyridazin-3-yl)phenol | |
| P-124 | N-(3-(4-(4-(3-amino-6-(2- | ¹H NMR (400 MHz, d₄-methanol) δ 14.23 - |
| | hydroxyphenyl)pyridazin-4-yl)-1,4- | 14.28 (m, 1 H) 9.16 - 9.21 (m, 1 H) 9.13 - 9.16 |
| | diazepan-1-yl)-6-ethoxypyrimidin-2- | (m, 1 H) 8.66 (dd, J = 4.0, 1.6 Hz, 1 H) 7.81 - |
| | yl)prop-2-yn-1-yl)pyrazolo[1,5- | 7.87 (m, 1 H) 7.49 (s, 1 H) 7.23 (s, 2 H) 6.82 - |
| | a]pyrimidine-2-carboxamide | 6.88 (m, 2 H) 6.17 (s, 1 H) 5.96 (s, 1 H) 4.33 |
| | | (d, J = 6.0 Hz, 2 H) 4.22 (q, J = 6.8 Hz, |
| | | 2 H) 3.37-3.43 (m, 2H) 3.31 (s, 2 H) 3.23 (br dd, |
| | | J = 5.2, 4.4 Hz, 2 H) 3.16 (d, J = 5.2 Hz, |
| | | 1H) 2.02 (br s, 2 H) 1.24 (t, J = 7.2 Hz, 3 H). |
| P-125 | 2-(6-amino-5-(4-(2-(3-hydroxyprop- | m/z (ESI⁺) 606 (M + H)⁺. |
| | 1-yn-1-yl)pyrimidin-4-yl)-1,4- | |
| | diazepan-1-yl)pyridazin-3-yl)phenol | |
| P-126 | N-(3-(4-(4-(3-amino-6-(2- | m/z (ESI⁺) 597 (M + H)⁺. |
| | hydroxyphenyl)pyridazin-4-yl)-1,4- | |
| | diazepan-1-yl)-6- | |
| | (methoxymethyl)pyrimidin-2-yl)prop- | |
| | 2-yn-1-yl)-1,4,5-trimethyl-1H- | |
| | pyrazole-3-carboxamide | |
| P-127 | N-(3-(4-(4-(3-amino-6-(2- | m/z (ESI⁺) 606 (M + H)⁺. |
| | hydroxyphenyl)pyridazin-4-yl)-1,4- | |
| | diazcpan-1-yl)-6- | |
| | (methoxymethyl)pyrimidin-2-yl)prop- | |
| | 2-yn-1-yl)pyrazolo[1,5-a]pyrimidine- | |
| | 2-carboxamide | |
| P-128 | 2-(6-amino-5-(4-(2-(3-(oxazol-2- | m/z (ESI⁺) 485 (M + H)⁺. |
| | yloxy)prop-1-yn-1-yl)pyrimidin-4- | |
| | yl)-1,4-diazepan-1-yl)pyridazin-3- | |
| | yl)phenol | |
| P-129 | N-(3-(4-(9-(3-amino-6-(2- | |
| | hydroxyphenyl)pyridazin-4-yl)-2,9- | m/z (ESI⁺) 605 (M + H)⁺. |
| | diazaspiro[5.5]undecan-2- | |
| | yl)pyrimidin-2-yl)prop-2-yn-1-yl)- | |
| | 5,6-dihydro-4H-pyrrolo[1,2- | |
| | b]pyrazole-2-carboxamide | |
| P-130 | 2-(6-amino-5-(3-(2-(3-((1-methyl-1H- | m/z (ESI⁺) 509 (M + H)⁺. |
| | pyrazol-3-yl)amino)prop-1-yn-1- | |
| | yl)pyrimidin-4-yl)-3,8- | |
| | diazabicyclo[3.2.1]octan-8- | |
| | yl)pyridazin-3-yl)phenol | |
| P-131 | 2-(6-amino-5-(4-(2-(3-((1-methyl-1H- | ¹H NMR (400 MHz, CDC1₃) δ 14.23 (br s, 1H), |
| | pyrazol-5-yl)oxy)prop-1-yn-1- | 8.43 - 8.23 (m, 1H), 7.80 (d, J = 7.2 Hz, 1H), |
| | yl)pyrimidin-4-yl)-1,4-diazepan-1- | 7.45 (s, 1H), 7.25 (d, J = 2.0 Hz, 1H), 7.24 - |
| | yl)pyridazin-3-yl)phenol | 7.20 (m, 1H), 6.88 - 6.83 (m, 2H), 6.68 - 6.62 |
| | | (m, 1H), 6.12 (s, 2H), 5.80 (d, J = 2.0 Hz, 1H), |
| | | 5.14 (s, 2H), 3.98 (s, 2H), 3.89 - 3.80 (m, 2H), |
| | | 3.59 (s, 3H), 3.51 - 3.47 (m, 2H), 3.37 - 3.33 |
| | | (m, 2H), 2.02 (s, 2H). |
| P-132 | 2-(5-(8-(2-(3-(1H- | m/z (ESI⁺) 531 (M + H)⁺. |
| | benzo[d][1,2,3]triazol-1-yl)prop-1- | |
| | yn-1-yl)pyrimidin-4-yl)-3,8- | |
| | diazabicyclo\| 3.2.1 \|octan-3-yl)-6- | |
| | aminopyridazin-3-yl)phenol | |
| P-133 | 2-(5-(8-(2-(3-(1H-pyrazol-1-yl)prop- | m/z (ESI⁺) 480 (M + H)⁺. |
| | 1-yn-1-yl)pyrimidin-4-yl)-3,8- | |
| | diazabicyclo[3.2.1]octan-3-yl)-6- | |
| | aminopyridazin-3-yl)phenol | |
| P-134 | 2-(6-amino-5-(8-(2-(3- | m/z (ESI⁺) 457 (M + H)⁺. |
| | (dimethylamino)prop-1-yn-1- | |
| | yl)pyrimidin-4-yl)-3,8- | |
| | diazabicyclo[3.2.1]octan-3- | |
| | yl)pyridazin-3-yl)phenol | |
| P-135 | 2-(6-amino-5-(4-(2-(imidazo[1,2- | m/z (ESI⁺) 505 (M + H)⁺. |
| | a]pyrazin-3-ylethynyl)pyrimidin-4- | |
| | yl)-1,4-diazepan-1-yl)pyridazin-3- | |
| | yl)phenol | |
| P-136 | 2-(6-amino-5-(4-(2-(3- | m/z (ESI⁺) 487 (M + H)⁺. |
| | morpholinoprop-1-yn-1-yl)pyrimidin- | |
| | 4-yl)-1,4-diazepan-1-yl)pyridazin-3- | |
| | yl)phenol | |
| P-137 | 4-(3-(4-(4-(3-amino-6-(2- | m/z (ESI⁺) 535 (M + H)⁺. |
| | hydroxyphenyl)pyridazin-4-yl)-1,4- | |
| | diazepan-1-yl)pyrimidin-2-yl)prop-2- | |
| | yn-1-yl)thiomorpholine 1,1-dioxide | |
| P-138 | 2-(6-amino-5-(4-(2-(3-(3-(pyridin-2- | m/z (ESI⁺) 545 (M + H)⁺. |
| | yl)-1H-pyrazol-1-yl)prop-1-yn-1- | |
| | yl)pyrimidin-4-yl)-1,4-diazepan-1- | |
| | yl)pyridazin-3-yl)phenol | |
| P-139 | N-(3-(4-(4-(3-amino-6-(2- | m/z (ESI⁺) 605.2 (M + H)⁺. |
| | hydroxyphenyl)pyridazin-4-yl)-1,4- | |
| | diazepan-1-yl)-6- | |
| | (dimethylamino)pyrimidin-2-yl)prop- | |
| | 2-yn-1-yl)pyrazolo[1,5-a]pyrimidine- | |
| | 2-carboxamide | |
| P-140 | N-(3-(4-(4-(3-amino-6-(2- | m/z (ESI⁺) 596.2 (M + H)⁺. |
| | hydroxyphenyl)pyridazin-4-yl)-1,4- | |
| | diazepan-1-yl)-6- | |
| | (dimethylamino)pyrimidin-2-yl)prop- | |
| | 2-yn-1-yl)-1,4,5-trimethyl-1H- | |
| | pyrazole-3-carboxamide | |
| P-141 | N-(3-(4-(4-(3-amino-6-(2- | m/z (ESI⁺) 582.2 (M + H)⁺. |
| | hydroxyphenyl)pyridazin-4-yl)-1,4- | |
| | diazepan-1-yl)-6- | |
| | (methylamino)pyrimidin-2-yl)prop-2- | |
| | yn-1-yl)-1,4,5-trimethyl-1H-pyrazole- | |
| | 3-carboxamide | |
| P-142 | tert-butyl (1-(3-(4-(4-(3-amino-6-(2- | m/z (ESI⁺) 600 (M + H)⁺. |
| | hydroxyphenyl)pyridazin-4-yl)-1,4- | |
| | diazepan-1-yl)pyrimidin-2-yl)prop-2- | |
| | yn-1-yl)piperidin-4-yl)carbamate | |
| P-143 | tert-butyl 4-(3-(4-(4-(3-amino-6-(2- | m/z (ESI⁺) 586 (M + H)⁺. |
| | hydroxyphenyl)pyridazin-4-yl)-1,4- | |
| | diazepan-1-yl)pyrimidin-2-yl)prop-2- | |
| | yn-1-yl)piperazine-1-carboxylate | |
| P-144 | 2-(5-(4-(2-(3-(1H-1,2,3-triazol-1- | ¹H NMR (400 MHz, d₄-methanol) δ 8.20 (s, |
| | yl)prop-1-yn-1-yl)pyrimidin-4-yl)- | 1H), 8.03 (d, J = 6.0 Hz, 1H), 7.80 (s, 1H), |
| | 1,4-diazepan-1-yl)-6-aminopyridazin- | 7.60 (d, J = 8.0 Hz, 1H), 7.43 (s, 1H), 7.23 (t, J |
| | 3-yl)phenol | = 7.6 Hz, 1H), 6.94 - 6.86 (m, 2H), 6.74 - 6.64 |
| | | (m, 1H), 5.58 (s, 2H), 4.20 - 3.91 (m, 2H), 3.88- |
| | | 3.68 (m, 2H), 3.59 (s, 2H), 3.46 - 3.37 (m, |
| | | 2H), 2.13 (s, 2H). |
| P-145 | 2-(5-(4-(2-(3-(1H-imidazol-1-yl)prop- | m/z (ESI⁺) 468.2 (M + H)⁺. |
| | 1-yn-1-yl)pyrimidin-4-yl)-1,4- | |
| | diazepan-1-yl)-6-aminopyridazin-3- | |
| | yl)phenol | |
| P-146 | 2-(6-amino-5-(4-(2-(3-(6,7- | ¹H NMR (400 MHz, d₄-methanol) δ 8.05 - 8.03 |
| | dihydropyrazolo[1,5-a]pyrazin-5(4H)- | (m, 1H), 7.65 - 7.60 (m, 1H), 7.44 (d, J = 9.2 |
| | yl)prop-1-yn-1-yl)pyrimidin-4-yl)- | Hz, 2H), 7.25 - 7.18 (m, 1H), 6.89 (d, J = 19.2 |
| | 1,4-diazepan-1-yl)pyridazin-3- | Hz, 2H), 6.73 - 6.66 (m, 1H), 6.11 - 6.08 (m, |
| | yl)phenol | 1H), 4.59 (s, 2H), 4.23 - 4.18 (m, 2H), 3.94 (s, |
| | | 3H), 3.82 (s, 3H), 3.56 (td, J = 1.6, 2.8 Hz, |
| | | 2H), 3.43 (s, 1H), 3.17 (t, J = 5.6 Hz, 3H), 2.09 |
| | | (d, J = 1.6 Hz, 2H). |
| P-147 | 2-(6-amino-5-(4-(2-(3- | m/z (ESI⁺) 431 (M + H)⁺. |
| | (methylamino)prop-1-yn-1- | |
| | yl)pyrimidin-4-yl)-1,4-diazepan-1- | |
| | yl)pyridazin-3-yl)phenol | |
| P-148 | 2-(5-(4-(2-(3-(1H-1,2,4-triazol-1- | ¹H NMR (400 MHz, d₄-methanol) δ 8.67 (s, |
| | yl)prop-1-yn-1-yl)pyrimidin-4-yl)- | 1H), 8.07 - 8.02 (m, 2H), 7.65 (d, J = 7.6 Hz, |
| | 1,4-diazepan-1-yl)-6-aminopyridazin- | 1H), 7.47 (s, 1H), 7.22 (t, J = 7.6 Hz, 1H), 6.92- |
| | 3-yl)phenol | 6.85 (m, 2H), 6.71 (td, J = 2.0, 3.9 Hz, 1H), |
| | | 5.38 (s, 2H), 4.22 - 3.89 (m, 2H), 3.87 - 3.66 |
| | | (m, 2H), 3.54 (s, 2H), 3.43 - 3.34 (m, 2H), 2.14 |
| | | (d, J = 5.2 Hz, 2H). |
| P-149 | 2-(5-(4-(2-(3-(1H-pyrazol-1-yl)prop- | ¹H NMR (400 MHz, d₆-DMSO) δ 8.10 (d, J = |
| | 1-yn-1-yl)pyrimidin-4-yl)-1,4- | 6.0 Hz, 1H), 7.83 (d, J = 2.4 Hz, 1H), 7.53 - |
| | diazepan-1-yl)-6-aminopyridazin-3- | 7.46 (m, 2H), 7.24 - 7.17 (m, 1H), 6.88 - 6.81 |
| | yl)phenol | (m, 2H), 6.73 (d, J = 6.4 Hz, 1H), 6.30 (t, J = |
| | | 2.0 Hz, 1H), 6.18 - 6.14 (m, 1H), 5.30 (s, 2H), |
| | | 4.09 - 3.93 (m, 1H), 3.92 - 3.57 (m, 2H), 3.44 - |
| | | 3.37 (m, 2H), 3.29 (s, 1H), 3.26 - 3.21 (m, 2H), |
| | | 2.07 - 1.96 (m, 2H). |
| P-150 | 2-(5-(4-(2-(3-(1H-benzo[d]imidazol- | ¹H NMR (400 MHz, d₄-methanol) δ 8.32 (d, J |
| | 1-yl)prop-1-yn-1-yl)pyrimidin-4-yl)- | = 3.2 Hz, 1H), 7.99 (d, J = 0.8 Hz, 1H), 7.78 - |
| | 1,4-diazepan-1-yl)-6-aminopyridazin- | 7.67 (m, 2H), 7.59 (d, J = 8.0 Hz, 1H), 7.41 (s, |
| | 3-yl)phenol | 1H), 7.38 - 7.28 (m, 2H), 7.19 (t, J = 7.6 Hz, |
| | | 1H), 6.88 (d, J = 8.0 Hz, 1H), 6.85 - 6.80 (m, |
| | | 1H), 6.68 - 6.60 (m, 1H), 5.40 (s, 2H), 4.16 - |
| | | 3.87 (m, 2H), 3.83 - 3.61 (m, 2H), 3.58 - 3.41 |
| | | (m, 2H), 3.33 (d, J = 3.6 Hz, 2H), 2.13 - 2.05 |
| | | (m, 2H). |
| P-151 | 2-(5-(4-(2-(3-(1H-indol-1-yl)prop-1- | ¹H NMR (400 MHz, d₄-methanol) δ 8.05 - 7.98 |
| | yn-1-yl)pyrimidin-4-yl)-1,4-diazepan- | (m, 1H), 7.64 (d, J = 7.8 Hz, 1H), 7.60 - 7.52 |
| | 1-yl)-6-aminopyridazin-3-yl)phenol | (m, 2H), 7.49 - 7.45 (m, 1H), 7.37 - 7.33 (m, |
| | | 1H), 7.27 - 7.18 (m, 2H), 7.12 - 7.04 (m, 1H), |
| | | 6.94 - 6.85 (m, 2H), 6.70 - 6.63 (m, 1H), 6.51 |
| | | (d, J = 3.0 Hz, 1H), 5.29 - 5.26 (m, 2H), 4.30 - |
| | | 3.98 (m, 2H), 3.84 - 3.67 (m, 2H), 3.52 (s, 2H), |
| | | 3.41 - 3.36 (m, 2H), 2.18 - 2.08 (m, 2H). |
| P-152 | 2-(6-amino-5-(4-(2-(3- | m/z (ESI⁺) 445 (M + H)⁺. |
| | (dimethylamino)prop-1-yn-1- | |
| | yl)pyrimidin-4-yl)-1,4-diazepan-1- | |
| | yl)pyridazin-3-yl)phenol | |
| P-153 | N-[3-(4-{(R)-4-[3-amino-6-(o- | m/z (ESI⁺) 489 (M + H)⁺. |
| | hydroxyphenyl)-4-pyridazinyl]-7- | |
| | methyl-1,4-diazepan-1-yl}-2- | |
| | pyrimidinyl)-2-propenyl]-N- | |
| | methylacetamide | |
| P-154 | o-{5-[(R)-4-(2-{3-[(2-fluoroethyl)-N- | m/z (ESI⁺) 491.2 (M + H)⁺. |
| | methylamino]-1-propynyl}-4- | |
| | pyrimidinyl)-5-methyl-1,4-diazepan- | |
| | 1-yl]-6-amino-3-pyridaziny1} phenol | |
| P-155 | o-{5-[(R)-4-(2-{3-[(2,2- | m/z (ESI⁺) 509.3 (M + H)⁺. |
| | difluoroethyl)-N-methylamino]-1- | |
| | propynyl}-4-pyrimidinyl)-5-methyl- | |
| | 1,4-diazepan-1-yl]-6-amino-3- | |
| | pyridazinyl}phenol | |
| P-156 | o-{5-[(1R,5S,8r)-3-{2-[3- | m/z (ESI⁺) 472.2 (M + H)⁺. |
| | (dimethylamino)-1-propynyl]-4- | |
| | pyrimidinyl}-3-azabicyclo[3.2.1]oct- | |
| | 8-yloxy]-6-amino-3- | |
| | pyridazinyl}phenol | |
| P-157 | o-[6-amino-5-(9-{2-[3- | m/z (ESI⁺) 471.2 (M + H)⁺. |
| | (dimethylamino)-1-propynyl]-4- | |
| | pyrimidinyl}-3,9- | |
| | diazabicyclo[4.2.1]non-3-yl)-3- | |
| | pyridazinyl]phenol | |
| P-158 | o-(6-amino-5-{4-[2-(1-propenyl)-4- | ¹H NMR (400 MHz, d₆-DMSO) δ 8.10 (d, J = |
| | pyrimidinyl]-1,4-diazepan-1-yl}-3- | 6.1 Hz, 1H), 7.81 (dd, J = 8.1, 1.6 Hz, 1H), |
| | pyridazinyl)phenol | 7.49 (s, 1H), 7.22 (ddd, J = 8.5, 7.2, 1.6 Hz, |
| | | 1H), 6.96 (dq, J = 15.5, 6.9 Hz, 1H), 6.89 - |
| | | 6.81 (m, 2H), 6.55 (d, J = 6.2 Hz, 1H), 6.29 |
| | | (dq, J = 15.4, 1.6 Hz, 1H), 6.16 (s, 2H), 4.26 - |
| | | 3.37 (m, 10H), 2.03 (p, J = 5.9 Hz, 2H), 1.88 |
| | | (dd, J = 6.9, 1.7 Hz, 3H). |
| P-159 | o-(6-amino-5-{4-[2-(3-methoxy-1- | ¹H NMR (400 MHz, d₆-DMSO) δ 8.15 - 8.11 |
| | propenyl)-4-pyrimidinyl]-1,4- | (m, 1H), 7.84 - 7.77 (m, 1H), 7.49 (s, 1H), |
| | diazepan-1-yl}-3-pyridazinyl)phenol | 7.22 (ddd, J = 8.4, 7.1, 1.6 Hz, 1H), 6.94 (dt, J |
| | | = 15.7, 5.2 Hz, 1H), 6.89 - 6.83 (m, 2H), 6.59 |
| | | (d, J = 6.2 Hz, 1H), 6.47 - 6.37 (m, 1H), 6.16 |
| | | (s, 2H), 4.10 (dd, J = 5.2, 1.8 Hz, 3H), 3.87 - |
| | | 3.50 (m, 5H), 3.45 (t, J = 5.3 Hz, 8H), 2.12 - |
| | | 1.95 (m, 2H) |
| P-160 | o-{5-[(R)-5-methyl-4-(2-{3-(3-oxa-8- | m/z (ESI⁺) 527.2 (M + H)⁺. |
| | azabicyclo[3.2.1]oct-8-yl)-1- | |
| | propynyl}-4-pyrimidinyl)-1,4- | |
| | diazepan-1-yl]-6-amino-3- | |
| | pyridazinyl}phenol | |
| P-161 | o-{5-[(R)-4-(2-{3-[2- | m/z (ESI⁺) 545.3 (M + H)⁺. |
| | (methoxymethyl)-4-morpholinyl]-1- | |
| | propynyl}-4-pyrimidinyl)-5-methyl- | |
| | 1,4-diazepan-1-yl]-6-amino-3- | |
| | pyridazinyl}phenol | |
| P-162 | o-{5-[(R)-4-(2-{3-[3- | m/z (ESI⁺) 545.2 (M + H)⁺. |
| | (mcthoxymcthyl)-4-morpholinyl]-1- | |
| | propynyl}-4-pyrimidinyl)-5-methyl- | |
| | 1,4-diazepan-1-yl]-6-amino-3- | |
| | pyridazinyl}phenol | |
| P-163 | o-{5-[(R)-4-(2-{3-[(2,2- | ¹H NMR (400 MHz, d₆-DMSO) δ 8.17 (s, 2H), |
| | difluoroethyl)-N-methylamino]-1- | 7.87 (dd, J = 8.1, 1.7 Hz, 1H), 7.53 (d, J = 16.3 |
| | propenyl}-4-pyrimidinyl)-5-methyl- | Hz, 1H), 7.22 (ddd, J = 8.4, 7.1, 1.6 Hz, 1H), |
| | 1,4-diazepan-1-yl]-6-amino-3- | 7.01 - 6.74 (m, 3H), 6.60 (d, J = 6.3 Hz, 1H), |
| | pyridazinyl}phenol | 6.42 (d, J = 15.5 Hz, 1H), 6.30 (d, J = 15.1 Hz, |
| | | 2H), 4.89 (s, 1H), 4.66 - 4.53 (m, 1H), 4.48 (t, |
| | | J = 5.2 Hz, 1H), 3.88 (d, J = 118.2 Hz, 3H), |
| | | 3.22 (d, J = 6.5 Hz, 4H), 3.07 - 2.88 (m, 2H), |
| | | 2.81 (t, J = 21.4 Hz, 2H), 2.67 (dt, J = 28.9, 5.5 |
| | | Hz, 2H), 2.24 (s, 3H), 2.04 (s, 1H), 1.15 (d, J = |
| | | 6.2 Hz, 3H). |
| P-164 | o-(5-[(R)-4-(2-{3-[(2-fluoroethyl)-N- | m/z (ESI⁺) 493 (M + H)⁺. |
| | methylamino]-1-propeny1}-4- | |
| | pyrimidinyl)-5-methyl-1,4-diazepan- | |
| | 1-yl\|-6-amino-3-pyridazinyl}phenol | |
| P-165 | o-{5-[(R)-5-methyl-4-{2-[3- | m/z (ESI⁺) 447 (M + H)⁺. |
| | (methylamino)-1-propenyl]-4- | |
| | pyrimidinyl}-1,4-diazepan-1-y1]-6- | |
| | amino-3-pyridaziny1}phenol | |
| P-166 | o-{5-[(R)-4-{2-[3-(1-azetidinyl)-1- | m/z (ESI⁺) 473 (M + H)⁺. |
| | propenyl]-4-pyrimidinyl}-5-methyl- | |
| | 1,4-diazepan-1-yl]-6-amino-3- | |
| | pyridazinyl}phenol | |
| P-167 | o-(5-{(R)-5-methyl-4-[2-(3- | ¹H NMR (499 MHz, d₆-DMSO) δ 8.15 (s, 1H), |
| | morpholino-1-propenyl)-4- | 7.87 (d, J = 7.9 Hz, 1H), 7.53 (d, J = 13.3 Hz, |
| | pyrimidinyl]-1,4-diazepan-1-y1}-6- | 1H), 7.22 (ddd, J = 8.5, 7.2, 1.6 Hz, 1H), 6.97 - |
| | amino-3-pyridazinyl)phenol | 6.79 (m, 3H), 6.60 (d, J = 6.3 Hz, 1H), 6.42 (d, |
| | | J = 15.5 Hz, 1H), 6.31 (d, J = 14.8 Hz, 2H), |
| | | 4.89 (s, 1H), 4.64 (d, J = 14.5 Hz, 1H), 3.99 (s, |
| | | 1H), 3.70 (d, J = 55.8 Hz, 2H), 3.54 - 3.22 (m, |
| | | 7H), 3.23 - 3.08 (m, 3H), 3.06 - 2.68 (m, 3H), |
| | | 2.39 (s, 4H), 2.24 (s, 2H), 2.08 (s, 1H), 1.15 (d, |
| | | J = 6.3 Hz, 3H). |
| P-168 | o-(5-{(R)-4-[2-(3-hydroxy-1- | m/z (ESI⁺) 434 (M + H)⁺. |
| | propenyl)-4-pyrimidinyl]-5-methyl- | |
| | 1,4-diazepan-1-y1}-6-amino-3- | |
| | pyridazinyl)phenol | |
| P-169 | o-(5-{(R)-5-methyl-4-[2-(1- | m/z (ESI⁺) 418 (M + H)⁺. |
| | propenyl)-4-pyrimidinyl]-1,4- | |
| | diazepan-1-yl}-6-amino-3- | |
| | pyridazinyl)phenol | |
| P-170 | o-(5-{(R)-4-[2-(3-methoxy-1- | ¹H NMR (499 MHz, d₆-DMSO) δ 8.16 (s, 1H), |
| | propenyl)-4-pyrimidinyl]-5-methyl- | 8.16 (s, 1H), 7.88 (d, J = 7.8 Hz, 2H), 7.54 (d, J |
| | 1,4-diazepan-1-yl}-6-amino-3- | = 15.4 Hz, 2H), 7.22 (ddd, J = 8.4, 7.2, 1.5 Hz, |
| | pyridazinyl)phenol | 2H), 6.95 (dt, J = 15.6, 5.2 Hz, 2H), 6.90 - |
| | | 6.82 (m, 3H), 6.61 (d, J = 6.3 Hz, 2H), 6.46 (dt, |
| | | J = 15.6, 1.9 Hz, 2H), 6.31 (d, J = 14.7 Hz, |
| | | 3H), 4.90 (s, 1H), 4.64 (d, J = 14.1 Hz, 1H), |
| | | 4.13-4.08 (m, 4H), 4.00 (s, 1H), 3.76 (s, 2H), |
| | | 3.30 (s, 5H), 2.98 (s, 1H), 2.88 (s, 1H), 2.79 (s, |
| | | 2H), 2.25 (s, 1H), 2.07 (s, 2H), 1.15 (d, J = 6.2 |
| | | Hz, 5H). |
| P-171 | o-(5-{(R)-4-[2-(2- | ¹H NMR (499 MHz, d₆-DMSO) δ 14.22 (s, |
| | cyclopropylethenyl)-4-pyrimidinyl]- | 1H), 8.09 (d, J = 5.9 Hz, 1H), 7.88 (dd, J = 8.0, |
| | 5-methyl-1,4-diazepan-1-yl}-6- | 1.6 Hz, 1H), 7.53 (s, 1H), 7.22 (ddd, J = 8.5, |
| | amino-3-pyridazinyl)phenol | 7.1, 1.6 Hz, 1H), 6.90 - 6.81 (m, 2H), 6.55 - |
| | | 6.44 (m, 2H), 6.35 (d, J = 15.4 Hz, 1H), 6.31 |
| | | (s, 2H), 4.88 (s, 0H), 4.62 (s, 0H), 3.98 (s, 0H), |
| | | 3.74 (s, 1H), 3.49 (s, 2H), 2.95 (s, 1H), 2.78 (s, |
| | | 1H), 2.24 (dt, J = 14.5, 7.0 Hz, 1H), 2.07 (s, |
| | | 1H), 1.65 (dd, J = 9.1, 4.6 Hz, 1H), 1.14 (d, J = |
| | | 6.3 Hz, 3H), 0.88 - 0.83 (m, 2H), 0.63 - 0.51 |
| | | (m, 2H). |
| P-172 | o-[6-amino-5-(1-{2-[3- | ¹H NMR (499 MHz, d₆-DMSO) δ 13.90 (s, |
| | (dimethylamino)-1-propynyl]-4- | 1H), 8.13 (s, 1H), 7.90 (d, J = 12.8 Hz, 2H), |
| | pyrimidinyl}-4-azepanyl)-3- | 7.25 - 7.18 (m, 1H), 6.91 - 6.82 (m, 2H), 6.69 |
| | pyridazinyl]phenol | (d, J = 3.8 Hz, 2H), 3.75 (s, 1H), 3.59 (s, 1H), |
| | | 3.49 (s, 2H), 2.88 (dt, J = 10.9, 5.6 Hz, 1H), |
| | | 2.27 (s, 5H), 2.08 (s, 1H), 1.99 (d, J = 13.9 Hz, |
| | | 1H), 1.82 (s, 3H). |
| P-173 | o-{5-[(R)-5-methyl-4-(2-{2-[1-(2,2,2- | m/z (ESI⁺) 539.2 (M + H)⁺. |
| | trifluoroethyl)-3-azetidinyl]ethynyl}- | |
| | 4-pyrimidinyl)-1,4-diazepan-1-y1]-6- | |
| | amino-3-pyridazinyl}phenol | |
| P-174 | o-{5-[(R)-4-(2-{2-[1-(2,2- | m/z (ESI⁺) 521.2 (M + H)⁺. |
| | difluoroethyl)-3-azetidinyl]ethynyl}- | |
| | 4-pyrimidinyl)-5-methyl-1,4- | |
| | diazepan-1-y1]-6-amino-3- | |
| | pyridazinyl}phenol | |
| P-175 | (R)-1-[3-(4-{(R)-4-[3-amino-6-(o- | m/z (ESI⁺) 531.2 (M + H)⁺. |
| | hydroxyphenyl)-4-pyridazinyl]-7- | |
| | methyl-1,4-diazepan-1-yl}-5-fluoro- | |
| | 2-pyrimidinyl)-2-propynyl]-5-methyl- | |
| | 2-pyrrolidinone | |
| P-176 | (S)-1-[3-(4-{(R)-4-[3-amino-6-(o- | m/z (ESI⁺) 531.2 (M + H)⁺. |
| | hydroxyphenyl)-4-pyridazinyl]-7- | |
| | methyl-1,4-diazepan-1-yl}-5-fluoro- | |
| | 2-pyrimidinyl)-2-propynyl]-5-methyl- | |
| | 2-pyrrolidinone | |
| P-177 | o-{5-[(R)-4-{2-[3-(3,5-dimethyl-1- | m/z (ESI⁺) 510.3 (M + H)⁺. |
| | pyrazolyl)-1-propynyl]-4- | |
| | pyrimidinyl}-5-methyl-1,4-diazepan- | |
| | 1-yl]-6-amino-3-pyridazinyl}phenol | |
| P-178 | o-{5-[(R)-5-methyl-4-(2-{3-(2-oxa-5- | m/z (ESI⁺) 527 (M + H)⁺. |
| | azabicyclo[2.2.2]oct-5-yl)-1- | |
| | propynyl}-4-pyrimidinyl)-1,4- | |
| | diazepan-1-yl]-6-amino-3- | |
| | pyridazinyl}phenol | |
| P-179 | o-{5-[(R)-5-methyl-4-(2-{3-(6-oxa-3- | m/z (ESI⁺) 513.3 (M + H)⁺. |
| | azabicyclo[3.1.1]hept-3-yl)-1- | |
| | propynyl}-4-pyrimidinyl)-1,4- | |
| | diazepan-1-yl]-6-amino-3- | |
| | pyridazinyl}phenol | |
| P-180 | o-{5-[(1R,5S)-7-{2-[3-(3,3-difluoro- | m/z (ESI⁺) 505.1 (M + H)⁺. |
| | 1-azetidinyl)-1-propynyl]-4- | |
| | pyrimidinyl}-3,7- | |
| | diazabicyclo\| 3.3.0\|oct-3-y1\|-6-amino- | |
| | 3-pyridaziny1} phenol | |
| P-181 | o-{5-[(1R,5S)-7-{2-[3- | m/z (ESI⁺) 457.2 (M + H)⁺. |
| | (dimethylamino)-1-propynyl]-4- | |
| | pyrimidinyl}-3,7- | |
| | diazabicyclo[3.3.0]oct-3-yl]-6-amino- | |
| | 3-pyridazinyl} phenol | |
| P-182 | o-{6-amino-5-[3-(2-{3-(2- | m/z (ESI⁺) 495.2 (M + H)⁺. |
| | azabicyclo[2.1.1]hex-2-yl)-1- | |
| | propynyl}-4-pyrimidinyl)-3,8- | |
| | diazabicyclo[3.2.1]oct-8-yl]-3- | |
| | pyridazinyl}phenol | |
| P-183 | o-{5-[(R)-5-methyl-4-(2-{3-(8-oxa-3- | ¹H NMR (400 MHz, d₄-methanol) δ |
| | azabicyclo[3.2.1]oct-3-yl)-1- | 8.17 - 8.00 (m, 1H), 7.75 (d, J = 7.6 Hz, 1H), |
| | propynyl}-4-pyrimidinyl)-1,4- | 7.50 (s, 1H), 7.24 (t, J = 7.6 Hz, 1H), 7.01 - |
| | diazepan-1-yl]-6-amino-3- | 6.83 (m, 2H), 6.73 (d, J = 6.4 Hz, 1H), 4.75 - |
| | pyridazinyl}phenol | 4.71 (m, 0.5H), 4.59 (d, J = 3.6 Hz, 0.5H), 4.32 |
| | | (s, 2H), 4.10 - 4.04 (m, 0.5H), 3.87 - 3.80 (m, |
| | | 0.5H), 3.67 - 3.56 (m, 2H), 3.50 (s, 2H), 3.35 |
| | | (s, 1H), 3.21 (s, 1H), 2.87 - 2.78 (m, 1H), 2.71- |
| | | 2.62 (m, 4H), 2.42 - 2.32 (m, 1H), 2.25 - 2.13 |
| | | (m, 1H), 1.99 (d, J = 6.5 Hz, 2H), 1.89 - 1.83 |
| | | (m, 2H), 1.26 (s, 3H). |
| P-184 | o-{5-[(R)-5-methyl-4-(2-{3-(3-oxa-6- | m/z (ESI⁺) 513.3 (M + H)⁺. |
| | azabicyclo[3.1.1]hept-6-yl)-1- | |
| | propynyl}-4-pyrimidinyl)-1,4- | |
| | diazepan-1-yl]-6-amino-3- | |
| | pyridazinyl}phenol | |
| P-185 | o-{5-[(R)-5-methyl-4-{2-[3-(2,4,5- | m/z (ESI⁺) 524.3 (M + H)⁺. |
| | trimethyl-1-imidazolyl)-1-propynyl]- | |
| | 4-pyrimidinyl}-1,4-diazepan-1-y1]-6- | |
| | amino-3-pyridaziny1} phenol | |
| P-186 | o-{5-[(R)-5-methyl-4-{2-[3-(2- | m/z (ESI⁺) 496.3 (M + H)⁺. |
| | methyl-1-imidazolyl)-1-propynyl]-4- | |
| | pyrimidinyl}-1,4-diazepan-1-yl]-6- | |
| | amino-3-pyridazinyl}phenol | |
| P-187 | o-{6-amino-5-[8-(2-{3-(2- | m/z (ESI⁺) 523.2 (M + H)⁺. |
| | azabicyclo[2.1.1]hex-2-yl)-1- | |
| | propynyl}-4-pyrimidinyl)-2,8-diaza- | |
| | 2-spiro[4.5]decyl]-3- | |
| | pyridazinyl}phenol | |
| P-188 | o-{6-amino-5-[8-(2-{3-(2- | m/z (ESI⁺) 495.2 (M + H)⁺. |
| | azabicyclo[2.1.1]hex-2-yl)-1- | |
| | propynyl}-4-pyrimidinyl)-3,8- | |
| | diazabicyclo[3.2.1]oct-3-yl]-3- | |
| | pyridazinyl}phenol | |
| P-189 | o-{5-[(R)-4-(2-{3-(2- | m/z (ESI⁺) 515.2 (M + H)⁺. |
| | azabicyclo[2.1.1]hex-2-yl)-1- | |
| | propynyl}-5-fluoro-4-pyrimidinyl)-5- | |
| | methyl-1,4-diazepan-1-yl]-6-amino-3- | |
| | pyridazinyl}phenol | |
| P-190 | o-{5-[(R)-5-methyl-4-(2-{3-(1- | m/z (ESI⁺) 511 (M + H)⁺. |
| | methyl-2-azabicyclo[2.1.1]hex-2-yl)- | |
| | 1-propynyl}-4-pyrimidinyl)-1,4- | |
| | diazepan-1-yl]-6-amino-3- | |
| | pyridazinyl}phenol | |
| P-191 | o-{5-[(R)-5-methyl-4-{2-[3-(1- | m/z (ESI⁺) 481.1 (M + H)⁺. |
| | pyrrolyl)-1-propynyl]-4-pyrimidinyl}- | |
| | 1,4-diazepan-1-yl]-6-amino-3- | |
| | pyridaziny1}phenol | |
| P-192 | o-{5-[(R)-5-methyl-4-{2-[2-(1- | ¹H NMR (499 MHz, d₆-DMSO) δ 8.12 (s, 1H), |
| | methyl-3-azetidinyl)ethynyl]-4- | 7.87 (s, 1H), 7.54 (d, J = 8.9 Hz, 1H), 7.22 (t, J |
| | pyrimidinyl}-1,4-diazepan-1-y1]-6- | = 7.6 Hz, 1H), 6.86 (dd, J = 10.8, 7.8 Hz, 2H), |
| | amino-3-pyridaziny1} phenol | 6.72 (d, J = 6.2 Hz, 1H), 6.32 (s, 2H), 6.27 (s, |
| | | 1H), 4.80 (s, 1H), 4.52 (d, J = 14.7 Hz, 0H), |
| | | 3.99 (s, 1H), 3.76 (s, 1H), 3.38 (s, 0H), 3.03 (s, |
| | | 3H), 2.84 (s, 1H), 2.79 - 2.71 (m, 1H), 2.22 (s, |
| | | 7H), 1.12 (s, 3H), 1.04 (d, J = 6.7 Hz, 1H). |
| P-193 | o-[6-amino-5-(3-{2-[3-(3-fluoro-3- | ¹H NMR (499 MHz, d₆-DMSO) δ 8.17 (d, J = |
| | methyl-1-pyrrolidinyl)-1-propynyl]-4- | 6.1 Hz, 1H), 7.97 (dd, J = 7.9, 1.6 Hz, 1H), |
| | pyrimidinyl}-3,8- | 7.47 (s, 1H), 7.24 (td, J = 7.6, 1.5 Hz, 1H), 6.89 |
| | diazabicyclo[3.2.1]oct-8-yl)-3- | (t, J = 7.8 Hz, 2H), 6.79 (d, J = 6.2 Hz, 1H), |
| | pyridazinyl]phenol | 6.21 (s, 2H), 4.47 (s, 2H), 4.47 (d, J = 7.6 Hz, |
| | | 1H), 3.62 (d, J = 1.6 Hz, 2H), 2.99 - 2.83 (m, |
| | | 3H), 2.69 - 2.60 (m, 1H), 2.63 - 2.57 (m, 1H), |
| | | 2.12 - 1.85 (m, 5H), 1.67 (d, J = 7.7 Hz, 2H), |
| | | 1.45 (d, J = 21.4 Hz, 4H). |
| P-194 | o-[6-amino-5-(8-{2-[3-(3-fluoro-3- | m/z (ESI⁺) 515 (M + H)⁺. |
| | methyl-1-pyrrolidinyl)-1-propynyl]-4- | |
| | pyrimidinyl}-3,8- | |
| | diazabicyclo[3.2.1]oct-3-yl)-3- | |
| | pyridazinyl]phenol | |
| P-195 | o-[6-amino-5-(8-{2-[3-(3-fluoro-3- | m/z. (ESI⁺) 543 (M + H)⁺. |
| | methyl-1-pyrrolidinyl)-1-propynyl]-4- | |
| | pyrimidinyl}-2,8-diaza-2- | |
| | spiro[4.5]decyl)-3-pyridazinyl]phenol | |
| P-196 | o-{5-[(R)-4-(2-{2-(5- | m/z (ESI⁺) 483 (M + H)⁺. |
| | azabicyclo[2.1.1]hex-1-yl)ethynyl}-4- | |
| | pyrimidinyl)-5-methyl-1,4-diazepan- | |
| | 1-yl]-6-amino-3-pyridazinyl}phenol | |
| P-197 | o-[6-amino-5-(3-(2-[3-(1-azetidinyl)- | ¹H NMR (400 MHz, d₆-DMSO) δ 8.22 - 8.02 |
| | 1-propynyl]-4-pyrimidinyl}-3- | (m, 1H), 7.95 (d, J = 8.0 Hz, 1H), 7.50 (s, 1H), |
| | azabicyclo[3.3.0]oct-7-yloxy)-3- | 7.37 - 7.18 (m, 1H), 6.89 (d, J = 8.0 Hz, 2H), |
| | pyridazinyl]phenol | 6.57 - 6.33 (m, 2H), 5.27 (s, 1H), 4.03 (s, 1H), |
| | | 3.47 (s, 9H), 2.97 - 2.76 (m, 2H), 2.67 (s, 4H), |
| | | 2.07 - 1.94 (m, 1H), 1.90 - 1.79 (m, 1H) |
| P-198 | o-{5-[(R)-4-{2-[(R)-3-hydroxy-1- | ¹H NMR (400 MHz, d₆-DMSO) δ 14.25 (d, J = |
| | butynyl]-4-pyrimidinyl}-5-methyl- | 7.6 Hz, 1H), 8.20 - 8.07 (m, 1H), 7.89 (dd, J = |
| | 1,4-diazepan-1-yl]-6-amino-3- | 7.6, 18.0 Hz, 1H), 7.54 (d, J = 10.0 Hz, 1H), |
| | pyridazinyl}phenol | 7.29 - 7.18 (m, 1H), 6.91 - 6.80 (m, 2H), 6.74 |
| | | (d, J = 6.4 Hz, 1H), 6.39 - 6.26 (m, 2H), 5.59 |
| | | (d, J = 5.6 Hz, 1H), 4.86 - 4.75 (m, 0.5H), 4.62- |
| | | 4.53 (m, 1H), 4.08 - 3.93 (m, 0.5H), 3.77 ( d, |
| | | J = 3.2 Hz, 1H), 3.57 - 3.40 (m, 3H), 3.03 - |
| | | 2.95 (m, 0.5H), 2.87 - 2.80 (m, 0.5H), 2.80 - |
| | | 2.70 (m, 1H), 2.34 - 2.20 (m, 1H), 2.19 - 1.97 |
| | | (m, 1H), 1.38 (d, J = 6.4 Hz, 3H), 1.15 - 1.10 |
| | | (m, 3H) |
| P-199 | o-{5-[(R)-5-methyl-4-(2-{3-(2-oxa-7- | m/z (ESI⁺) 541.2 (M + H)⁺. |
| | aza-7-spiro[3.5]nonyl)-1-propynyl}- | |
| | 4-pyrimidinyl)-1,4-diazepan-1-y1]-6- | |
| | amino-3-pyridazinyl}phenol | |
| P-200 | o-{5-[(R)-4-[2-(3-{(1S,4S)-2-oxa-5- | m/z (ESI⁺) 513.3 (M + H)⁺. |
| | azabicyclo[2.2.1]hept-5-yl}-1- | |
| | propynyl)-4-pyrimidinyl]-5-methyl- | |
| | 1,4-diazepan-1-yl\|-6-amino-3- | |
| | pyridazinyl}phenol | |
| P-201 | o-{5-[(R)-4-(2-{3-(5,5-difluoro-2- | m/z (ESI⁺) 547 (M + H)⁺. |
| | aza-2-spiro[3.3]hcptyl)-1-propynyl}- | |
| | 4-pyrimidinyl)-5-methyl-1,4- | |
| | diazepan-1-yl]-6-amino-3- | |
| | pyridazinyl}phenol | |
| P-202 | o-{5-[(R)-4-{2-[3-(dicthylamino)-1- | m/z (ESI⁺) 487 (M + H)⁺. |
| | propynyl]-4-pyrimidinyl}-5-methyl- | |
| | 1,4-diazepan-1-yl]-6-amino-3- | |
| | pyridazinyl}phenol | |
| P-203 | 1-[3-(4-{(R)-4-[3-amino-6-(o- | m/z (ESI⁺) 517 (M + H)⁺. |
| | hydroxyphenyl)-4-pyridazinyl]-7- | |
| | methyl-1,4-diazepan-1-y1}-5-fluoro- | |
| | 2-pyrimidinyl)-2-propynyl]-2- | |
| | pyrrolidinone | |
| P-204 | o-[6-amino-5-(6-{2-[3-(3,3-difluoro- | ¹H NMR (499 MHz, d₆-DMSO) δ 8.19 (d, J = |
| | 1-azetidinyl)-1-propynyl]-4- | 6.0 Hz, 1H), 7.90 - 7.85 (m, 1H), 7.35 (s, 1H), |
| | pyrimidinyl}-3,6- | 7.26 - 7.19 (m, 1H), 6.89 - 6.83 (m, 2H), 6.65 |
| | diazabicyclo[3.1.1]hept-3-yl)-3- | (d, J = 5.9 Hz, 1H), 6.01 (s, 2H), 4.62 (s, 2H), |
| | pyridazinyl]phenol | 3.73 - 3.65 (m, 7H), 3.64 - 3.52 (m, 1H), 3.4o- |
| | | 3.34 (m, 1H), 3.32 - 3.27 (m, 1H), 2.72 (d, J |
| | | = 8.0 Hz, 1H), 2.07 (d, J = 8.6 Hz, 1H), 1.90 (s, |
| | | 1H), 1.24 (s, 2H). |
| P-205 | o-[6-amino-5-(6-{2-[3-(1H-1,2,4- | ¹H NMR (499 MHz, d₆-DMSO) δ 8.64 (s, 1H), |
| | triazol-1-yl)-1-propynyl]-4- | 8.18 (d, J = 5.9 Hz, 1H), 8.07 (s, 1H), 7.88 (dd, |
| | pyrimidinyl}-3,6- | J = 8.3, 1.6 Hz, 1H), 7.35 (s, 1H), 7.22 (td, J = |
| | diazabicyclo[3.1.1]hept-3-yl)-3- | 7.7, 1.5 Hz, 1H), 6.90 - 6.83 (m, 2H), 6.66 (d, |
| | pyridazinyl]phenol | J = 5.9 Hz, 1H), 6.02 (s, 2H), 5.43 (s, 2H), 4.62 |
| | | (d, J = 5.9 Hz, 2H), 3.82 (s, 2H), 3.69 (s, 2H), |
| | | 2.71 (dt, J = 8.3, 6.1 Hz, 1H), 2.08 (d, J = 8.5 |
| | | Hz, 1H). |
| P-206 | o-[6-amino-5-(6-{2-[3- | ¹H NMR (499 MHz, d₆-DMSO) δ 8.17 (d, J = |
| | (dimethylamino)-1-propynyl]-4- | 5.9 Hz, 1H), 7.88 (dd, J = 8.4, 1.7 Hz, 1H), |
| | pyrimidinyl}-3,6- | 7.34 (s, 1H), 7.26 - 7.19 (m, 1H), 6.90 - 6.83 |
| | diazabicyclo[3.1.1]hcpt-3-yl)-3- | (m, 2H), 6.63 (d, J = 5.9 Hz, 1H), 6.01 (s, 2H), |
| | pyridazinyl]phenol | 4.62 (d, J = 5.9 Hz, 2H), 3.75 (s, 6H), 3.44 (s, |
| | | 2H), 2.72 (dt, J = 8.5, 6.2 Hz, 1H), 2.22 (s, |
| | | 6H), 2.07 (d, J = 8.4 Hz, 1H). |
| P-207 | o-(5-{(R)-4-[5-fluoro-2-(3-hydroxy- | ¹H NMR (400 MHz, d₆-DMSO) δ 14.23 (s, |
| | 1-propynyl)-4-pyrimidinyl]-5-methyl- | 1H), 8.21 (d, J = 7.2 Hz, 1H), 7.91 - 7.86 (m, |
| | 1,4-diazepan-1-yl}-6-amino-3- | 1H), 7.65 - 7.59 (m, 1H), 7.58 - 7.50 (m, 1H), |
| | pyridazinyl)phenol | 7.26 - 7.19 (m, 1H), 6.91 - 6.83 (m, 2H), 6.30 |
| | | (s, 2H), 5.43 (t, J = 6.0 Hz, 1H), 4.60 (d, J = |
| | | 9.2 Hz, 1H), 4.29 (d, J = 6.0 Hz, 2H), 4.23 - |
| | | 4.08 (m, 1H), 3.73 - 3.62 (m, 1H), 3.58 (d, J = |
| | | 12.4 Hz, 1H), 3.48 (dd, J = 7.6, 13.6 Hz, 1H), |
| | | 3.07 (t, J = 11.2 Hz, 1H), 2.90 (dd, J = 9.6, |
| | | 13.6 Hz, 1H), 2.31 - 2.19 (m, 1H), 2.12 - 1.97 |
| | | (m, 1H), 1.20 (d, J = 6.0 Hz, 3H) |
| P-208 | o-{5-[(R)-4-{2-[3-(dimethylamino)-1- | ¹H NMR (400 MHz, d₄-Methanol) δ 8.05 (d, J |
| | butynyl\|-5-fluoro-4-pyrimidinyl}-5- | = 6.4 Hz, 1H), 7.75 (d, J = 7.6 Hz, 1H), 7.51 |
| | methyl-1,4-diazepan-1-yl]-6-amino-3- | (s, 1H), 7.24 (t, J = 7.6 Hz, 1H), 6.97 - 6.83 (m, |
| | pyridaziny1}phenol | 2H), 4.76 - 4.61 (m, 1H), 4.45 - 4.25 (m, 1H), |
| | | 3.84 - 3.65 (m, 2H), 3.64 - 3.52 (m, 2H), 3.31 - |
| | | 3.25 (m, 1H), 3.00 - 2.84 (m, 1H), 2.37 (s, 6H), |
| | | 2.35 - 2.27 (m, 1H), 2.22 - 2.07 (m, 1H), 1.42 |
| | | (d, J = 7.2 Hz, 3H), 1.32 (d, J = 6.4 Hz, 3H) |
| P-209 | o-{5-[(R)-4-(2-{3-(8,8-difluoro-6- | ¹H NMR (499 MHz, d₆-DMSO) δ 14.24 (s, |
| | aza-6-spiro[3.4]octyl)-1-propyny1}-4- | 1H), 8.14 (s, 1H), 7.88 (s, 1H), 7.55 (s, 1H), |
| | pyrimidinyl)-5-methyl-1,4-diazepan- | 7.52 (s, 1H), 7.22 (ddd, J = 8.4, 7.1, 1.5 Hz, |
| | 1-yl]-6-amino-3-pyridaziny1} phenol | 1H), 6.90 - 6.82 (m, 2H), 6.75 (d, J = 6.3 Hz, |
| | | 1H), 6.34 (s, 1H), 6.27 (s, 1H), 4.78 (s, 1H), |
| | | 4.51 (d, J = 14.8 Hz, 0H), 4.00 (s, 1H), 3.77 (s, |
| | | 1H), 3.66 (s, 2H), 3.50 (d, J = 13.4 Hz, 2H), |
| | | 3.43 (s, 1H), 3.06 (t, J = 14.5 Hz, 2H), 2.94 (s, |
| | | 2H), 2.76 (dd, J = 13.9, 9.5 Hz, 1H), 2.25 (d, J |
| | | = 7.6 Hz, 1H), 2.14 (s, 1H), 2.04 (s, 0H), 1.89 |
| | | (s, 1H), 1.83 (s, 4H), 1.74 (s, 1H), 1.13 (s, 3H). |
| P-210 | o-{5-[(R)-4-{2-[3-(3-fluoro-3-methyl- | ¹H NMR (499 MHz, d₆-DMSO) δ 14.23 (s, |
| | 1-pyrrolidinyl)-1-propynyl]-4- | 1H), 8.13 (d, J = 9.2 Hz, 1H), 7.88 (dd, J = |
| | pyrimidinyl}-5-methyl-1,4-diazepan- | 16.0, 7.7 Hz, 1H), 7.54 (d, J = 15.6 Hz, 1H), |
| | 1-yl]-6-amino-3-pyridaziny1} phenol | 7.22 (ddd, J = 8.4, 7.1, 1.5 Hz, 1H), 6.90 - 6.82 |
| | | (m, 2H), 6.74 (d, J = 6.4 Hz, 1H), 6.33 (s, 1H), |
| | | 6.26 (s, 1H), 4.79 (s, 1H), 4.52 (d, J = 14.6 Hz, |
| | | 1H), 3.99 (s, 1H), 3.77 (s, 1H), 3.62 (s, 2H), |
| | | 3.02 (d, J = 28.0 Hz, 1H), 2.95 (d, J = 10.8 Hz, |
| | | 1H), 2.93 - 2.83 (m, 2H), 2.76 (dd, J = 14.0, |
| | | 9.5 Hz, 1H), 2.66 (d, J = 10.8 Hz, 1H), 2.61 (d, |
| | | J = 9.4 Hz, 2H), 2.24 (d, J = 9.4 Hz, 1H), 2.04 |
| | | (ddt, J = 20.3, 13.7, 6.0 Hz, 1H), 1.92 (ddt, J = |
| | | 23.5, 14.0, 7.3 Hz, 1H), 1.47 (d, J = 3.7 Hz, |
| | | 2H), 1.43 (d, J = 3.7 Hz, 2H), 1.13 (t, J = 5.0 |
| | | Hz, 3H). |
| P-211 | o-{5-[(R)-5-methyl-4-{2-[2- | ¹H NMR (499 MHz, d₆-DMSO) δ 14.22 (s, |
| | (tetrahydro-2-furyl)ethynyl]-4- | 1H), 8.14 (s, 1H), 7.86 (d, J = 7.9 Hz, 1H), |
| | pyrimidinyl}-1,4-diazepan-1-yl]-6- | 7.55 (s, 1H), 7.22 (ddd, J = 8.4, 7.2, 1.5 Hz, |
| | amino-3-pyridaziny1} phenol | 1H), 6.90 - 6.83 (m, 2H), 6.77 (s, 1H), 6.33 (s, |
| | | 1H), 6.27 (s, 1H), 4.78 (s, 2H), 4.53 (d, J = |
| | | 14.6 Hz, 0H), 4.00 (s, 1H), 3.84 (q, J = 7.1 Hz, |
| | | 1H), 3.75 (dd, J = 13.5, 7.5 Hz, 2H), 3.51 (s, |
| | | 2H), 3.42 (s, 1H), 2.99 (s, 0H), 2.75 (dd, J = |
| | | 13.9, 9.5 Hz, 1H), 2.23 (s, 2H), 1.96 (s, 4H), |
| | | 1.88 (s, 1H), 1.13 (d, J = 6.1 Hz, 3H). |
| P-212 | o-{6-amino-5-[1-(1-{2-[3-(1- | ¹H NMR (499 MHz, d₆-DMSO) δ 13.77 (s, |
| | imidazolyl)-1-propynyl]-4- | 1H), 8.62 (s, 1H), 8.29 (s, 1H), 8.24 - 8.16 (m, |
| | pyrimidinyl}-3-azetidinyl)-4- | 2H), 8.00 (dd, J = 8.3, 1.7 Hz, 1H), 7.27 (td, J |
| | pyrazolyl]-3-pyridazinyl} phenol | = 7.6, 1.6 Hz, 1H), 6.96 - 6.89 (m, 3H), 6.58 - |
| | | 6.50 (m, 3H), 5.50 (ddd, J = 13.2, 8.0, 5.3 Hz, |
| | | 1H), 5.22 (s, 2H), 4.58 (t, J = 8.7 Hz, 2H), 4.41 |
| | | (dd, J = 9.8, 5.2 Hz, 2H). |
| P-213 | o-{5-[(R)-5-methyl-4-(2-{3-(2-oxa-6- | ¹H NMR (499 MHz, d₆-DMSO) δ 14.24 (s, |
| | aza-6-spiro[3.3]heptyl)-1-propynyl}- | 1H), 8.13 (q, J = 6.2 Hz, 1H), 7.91 - 7.84 (m, |
| | 4-pyrimidinyl)-1,4-diazepan-1-yl]-6- | 1H), 7.54 (d, J = 16.8 Hz, 1H), 7.22 (ddd, J = |
| | amino-3-pyridazinyl}phenol | 8.4, 7.2, 1.6 Hz, 1H), 6.90 - 6.82 (m, 2H), 6.74 |
| | | (d, J = 6.4 Hz, 1H), 6.33 (s, 1H), 6.28 (d, J = |
| | | 14.0 Hz, 1H), 4.79 (s, 1H), 4.69 (s, 0H), 4.61 |
| | | (s, 4H), 4.52 (d, J = 15.2 Hz, 1H), 4.09 (d, J = |
| | | 2.4 Hz, 0H), 4.00 (s, 1H), 3.77 (s, 1H), 3.51 (s, |
| | | 0H), 3.47 (d, J = 15.0 Hz, 1H), 3.40 (d, J = 3.3 |
| | | Hz, 6H), 2.98 (s, 0H), 2.84 (s, 0H), 2.76 (dd, J |
| | | = 13.9, 9.5 Hz, 1H), 2.23 (s, 1H), 2.04 (s, 1H), |
| | | 1.13 (q, J = 5.3 Hz, 4H). |
| P-214 | o-{5-[(R)-4-{5-fluoro-2-[3-(1H-1,2,4- | m/z (ESI⁺) 501 (M + H)⁺. |
| | triazol-1-yl)-1-propynyl]-4- | |
| | pyrimidinyl}-5-methyl-1,4-diazepan- | |
| | 1-yl]-6-amino-3-pyridazinyl }phenol | |
| P-215 | 1-[3-(4-{ (R)-4-[3-amino-6-(o- | m/z (ESI⁺) 496 (M + H)⁺. |
| | hydroxyphenyl)-4-pyridazinyl]-7- | |
| | methyl-1,4-diazepan-1-yl}-2- | |
| | pyrimidinyl)-2-propynyl]-3- | |
| | azetidinecarbonitrile | |
| P-216 | o-{6-amino-5-[1-(1-{2-[3- | m/z (ESI⁺) 468 (M + H)⁺. |
| | (dimethylamino)-1-propynyl]-4- | |
| | pyrimidinyl }-3-azetidinyl)-4- | |
| | pyrazolyl]-3-pyridazinyl} phenol | |
| P-217 | o-{5-[(S)-4-(2-{3-[N-methyl(2,2,2- | m/z (ESI⁺) 527 (M + H)⁺. |
| | trifluoroethyl)amino]-1-propynyl}-4- | |
| | pyrimidinyl)-5-methyl-1,4-diazepan- | |
| | 1-yl]-6-amino-3-pyridaziny1}phenol | |
| P-218 | o-{5-[(R)-4-{2-[(E)-3- | ¹H NMR (400 MHz, d₄-Methanol) δ 8.52 (s, 1 |
| | (dimcthylamino)-1-propcnyl]-4- | H), 8.16 (d, J = 6.4 Hz, 1 H), 7.78 - 7.72 (m, 1 |
| | pyrimidinyl }-5-methyl-1,4-diazepan- | H), 7.50 - 7.48 (m, 1 H), 7.25 - 7.22 (m, 1 H), |
| | 1-yl]-6-amino-3-pyridazinyl } phenol | 6.91 - 6.89 (m, 2 H), 6.74 - 6.68 (m, 2 H), 5.14- |
| | | 5.13 (m, 1 H), 4.18 - 4.08 (m, 1 H), 3.62 (d, J |
| | | = 7.2 Hz, 2 H), 3.63 - 3.55 (m, 3 H), 3.21 - |
| | | 3.09 (m, 1 H), 2.86 (s, 4 H), 2.77 - 2.63 (m, 5 |
| | | H), 2.46 - 2.30 (m, 1 H), 2.27 - 2.07 (m, 1 H), |
| | | 1.28-1.25 (m, 3 H). |
| P-219 | o-[6-amino-5-(3-{2-[3-(1- | ¹H NMR (400 MHz, d₄-Methanol) δ 8.04 (d, J |
| | imidazolyl)-1-propynyl]-4- | = 6.4 Hz, 1H), 7.85 - 7.78 (m, 2H), 7.43 (s, |
| | pyrimidinyl }-3-azabicyclo[3.3.0]oct- | 1H), 7.32 - 7.29 (m, 1H), 7.28 - 7.22 (m, 1H), |
| | 7-yloxy)-3-pyridazinyl]phenol | 7.02 (s, 1H), 6.97 - 6.89 (m, 2H), 6.48 (d, J = |
| | | 6.4 Hz, 1H), 5.26 (tt, J = 3.6, 6.4 Hz, 1H), 5.17 |
| | | (s, 2H), 3.91 - 3.44 (m, 4H), 3.09 - 2.95 (m, |
| | | 2H), 2.57 (td, J = 7.2, 14.4 Hz, 2H), 2.05 - 1.96 |
| | | (m, 2H) |
| P-220 | o-(6-amino-5-{3-[2-(3-morpholino-1- | ¹H NMR (400 MHz, d₄-Methanol) δ 8.04 (d, J |
| | propynyl)-4-pyrimidinyl]-3- | = 6.4 Hz, 1H), 7.85 - 7.78 (m, 2H), 7.43 (s, |
| | azabicyclo[3.3.0]oct-7-yloxy}-3- | 1H), 7.32 - 7.29 (m, 1H), 7.28 - 7.22 (m, 1H), |
| | pyridazinyl)phenol | 7.02 (s, 1H), 6.97 - 6.89 (m, 2H), 6.48 (d, J = |
| | | 6.4 Hz, 1H), 5.26 (tt, J = 3.6, 6.4 Hz, 1H), 5.17 |
| | | (s, 2H), 3.91 - 3.44 (m, 4H), 3.09 - 2.95 (m, |
| | | 2H), 2.57 (td, J = 7.2, 14.4 Hz, 2H), 2.05 - 1.96 |
| | | (m, 2H) |
| P-221 | 4-(3-{2-[3-(tert- | ¹H NMR (400 MHz, d₄-Methanol) δ 8.01 (d, J |
| | butoxycarbonylamino)-1-propynyl]- | = 6.4 Hz, 1H), 7.79 (d, J = 8.0 Hz, 1H), 7.43 |
| | 4-pyrimidinyl}-3- | (s, 1H), 7.30 - 7.22 (m, 1H), 6.97 - 6.89 (m, |
| | azabicyclo[3.3.0]oct-7-yloxy)-6-(o- | 2H), 6.45 (d, J = 6.4 Hz, 1H), 5.26 (td, J = 3.2, |
| | hydroxyphenyl)-3-pyridazinamine | 6.4 Hz, 1H), 4.07 (s, 2H), 3.87 - 3.46 (m, 4H), |
| | | 3.02 (s, 2H), 2.63 - 2.51 (m, 2H), 2.06 - 1.96 |
| | | (m, 2H), 1.45 (s, 9H) |
| P-222 | o-[6-amino-5-(3-{2-[3- | ¹H NMR (400 MHz, d₄-Methanol) δ 8.05 (s, |
| | (dimethylamino)-1-propynyl]-4- | 1H), 7.78 (d, J = 7.2 Hz, 1H), 7.41 (s, 1H), |
| | pyrimidinyl}-3-azabicyclo[3.3.0]oct- | 7.25 (s, 1H), 6.92 (d, J = 6.4 Hz, 2H), 6.48 (s, |
| | 7-yloxy)-3-pyridazinyl]phenol | 1H), 5.25 (s, 1H), 3.76 (s, 2H), 3.74 - 3.39 (m, |
| | | 4H), 3.01 (d, J = 10.4 Hz, 2H), 2.65 - 2.45 (m, |
| | | 8H), 2.06 - 1.93 (m, 2H) |
| P-223 | o-{5-[(R)-4-(2-{3-[(2R,4S)-2,4- | m/z (ESI⁺) 499 (M + H)⁺. |
| | dimethyl-1-azetidinyl]-1-propynyl }- | |
| | 4-pyrimidinyl)-5-methyl-1,4- | |
| | diazepan-1-y1]-6-amino-3- | |
| | pyridazinyl }phenol | |
| P-224 | o-[6-amino-5-(8-{5-fluoro-2-[3-(1- | ¹H NMR (499 MHz, d₆-DMSO) δ 14.04 (s, |
| | imidazolyl)-1-propynyl]-4- | 2H), 8.29 (s, 1H), 7.92 (d, J = 7.7 Hz, 1H), |
| | pyrimidinyl}-3,8- | 7.57 (s, 1H), 7.23 (t, J = 7.6 Hz, 1H), 6.86 (dd, |
| | diazabicyclo[3.2.1]oct-3-yl)-3- | J = 11.7, 7.5 Hz, 3H), 6.04 (s, 3H), 5.25 (s, |
| | pyridazinyl]phenol | 2H), 4.86 (s, 2H), 3.07 (d, J = 11.6 Hz, 2H), |
| | | 2.21 (d, J = 7.6 Hz, 3H), 2.02 - 1.96 (m, 3H). |
| P-225 | o-[6-amino-5-(8-{2-[3- | ¹H NMR (499 MHz, d₆-DMSO) δ 14.14 (s, |
| | (dimethylamino)-1-propynyl]-5- | 1H), 8.28 (s, 1H), 7.92 (dd, J = 8.0, 1.6 Hz, |
| | fluoro-4-pyrimidinyl}-3,8- | 1H), 7.58 (s, 1H), 7.27 - 7.19 (m, 1H), 6.90 - |
| | diazabicyclo[3.2.1]oct-3-yl)-3- | 6.82 (m, 2H), 6.04 (s, 2H), 4.87 (s, 2H), 3.53 |
| | pyridazinyl]phenol | (s, 2H), 3.40 (dd, J = 11.9, 2.2 Hz, 2H), 3.08 |
| | | (d, J = 11.6 Hz, 2H), 2.29 (s, 6H), 2.25 - 2.15 |
| | | (m, 2H), 2.00 (dd, J = 7.7, 3.9 Hz, 2H) |
| P-226 | o-{5-[(R)-4-{2-[3-(dimethylamino)-1- | ¹H NMR (499 MHz, d₆-DMSO) δ 8.19 (d, J = |
| | propynyl]-5-fluoro-4-pyrimidinyl}-5- | 7.2 Hz, 1H), 7.88 (dd, J = 8.0, 1.6 Hz, 1H), |
| | mcthyl-1,4-diazcpan-1-yl]-6-amino-3- | 7.53 (s, 1H), 7.23 (ddd, J = 8.5, 7.1, 1.6 Hz, |
| | pyridazinyl }phenol | 1H), 6.87 (t, J = 8.0 Hz, 2H), 6.31 (s, 2H), 4.57 |
| | | (s, 1H), 4.17 (s, 1H), 3.67 (s, 2H), 3.57 (dd, J = |
| | | 13.6, 4.2 Hz, 1H), 3.48 (dd, J = 13.7, 7.9 Hz, |
| | | 1H), 3.10 - 3.00 (m, 1H), 2.90 (dd, J = 13.9, |
| | | 9.6 Hz, 1H), 2.30 - 2.24 (m, 0H), 2.23 (s, 6H), |
| | | 2.10 - 1.99 (m, 1H), 1.20 (d, J = 6.2 Hz, 3H). |
| P-227 | o-{5-[(R)-4-{2-[3-(1-imidazolyl)-1- | ¹H NMR (499 MHz, d₆-DMSO) δ 14.08 (s, |
| | propynyl]-5-methyl-4-pyrimidinyl}- | 1H), 7.99 (s, 1H), 7.88 (d, J = 7.8 Hz, 1H), |
| | 5-methyl-1,4-diazepan-1-yl]-6-amino- | 7.51 (s, 1H), 7.22 (t, J = 7.6 Hz, 1H), 6.86 (t, J |
| | 3-pyridaziny1} phenol | = 7.9 Hz, 2H), 6.27 (s, 2H), 5.20 (s, 2H), 4.71 |
| | | (t, J = 5.6 Hz, 1H), 4.30 (dd, J = 16.2, 4.2 Hz, |
| | | 1H), 3.59 (dd, J = 15.7, 9.7 Hz, 1H), 3.54 - |
| | | 3.43 (m, 2H), 3.09 (t, J = 11.2 Hz, 1H), 2.89 |
| | | (dd, J = 13.8, 9.5 Hz, 1H), 2.35 (s, 3H), 2.24 |
| | | (dt, J = 14.6, 7.0 Hz, 1H), 2.00 (dt, J = 14.9, |
| | | 10.0 Hz, 1H), 1.16 (d, J = 6.2 Hz, 3H). |
| P-228 | o-{5-[(R)-4-(2-{3-(8- | m/z (ESI⁺) 525 (M + H)⁺. |
| | azabicyclo[3.2.1]oct-8-yl)-1- | |
| | propynyl }-4-pyrimidinyl)-5-methyl- | |
| | 1,4-diazepan-1-y1]-6-amino-3- | |
| | pyridazinyl }phenol | |
| P-229 | o-{5-[(R)-4-{2-[3-(dimethylamino)-1- | ¹H NMR (499 MHz, d₆-DMSO) δ 14.18 (s, |
| | propynyl]-5-methyl-4-pyrimidinyl}- | 1H), 7.97 (s, 1H), 7.88 (dd, J = 8.0, 1.6 Hz, |
| | 5-methyl-1,4-diazepan-1-yl]-6-amino- | 1H), 7.52 (s, 1H), 7.22 (ddd, J = 8.5, 7.2, 1.6 |
| | 3-pyridaziny1}phenol | Hz, 1H), 6.90 - 6.83 (m, 2H), 6.27 (s, 2H), |
| | | 4.69 (dt, J = 12.3, 6.2 Hz, 1H), 4.30 (dd, J = |
| | | 15.5, 4.4 Hz, 1H), 3.59 (dd, J = 15.6, 9.7 Hz, |
| | | 1H), 3.49 (tt, J = 13.6, 6.1 Hz, 2H), 3.43 (s, |
| | | 2H), 3.12 (dd, J = 12.8, 9.7 Hz, 1H), 2.96 - |
| | | 2.87 (m, 1H), 2.35 (s, 3H), 2.23 (s, 6H), 2.00 |
| | | (dt, J = 15.1, 10.0 Hz, 1H), 1.18 (d, J = 6.2 Hz, |
| | | 3H). |
| P-230 | o-{5-[(R)-4-{2-[3-(3-fluoro-1- | m/z (ESI⁺) 489 (M + H)⁺. |
| | azetidinyl)-1-propynyl]-4- | |
| | pyrimidinyl }-5-methyl-1,4-diazepan- | |
| | 1-y1]-6-amino-3-pyridaziny1 }phenol | |
| P-231 | o-{5-[(R)-5-methyl-4-{2-[2-(1- | ¹H NMR (400 MHz, d₆-DMSO) δ 14.53 - |
| | methyl-2-piperidyl)ethynyl]-4- | 13.78 (m, 1H), 8.13 (s, 1H), 7.87 (s, 1H), 7.54 |
| | pyrimidinyl}-1,4-diazepan-1-yl]-6- | (br s, 1H), 7.55 (s, 1H), 7.19 - 7.25 (m, 2H), |
| | amino-3-pyridaziny1} phenol | 6.91 - 6.82 (m, 2H), 6.74 (d, J = 5.2 Hz, 1H), |
| | | 6.45 - 6.15 (m, 2H), 4.90 - 4.45 (m, 1H), 4.20 - |
| | | 3.91 (m, 1H), 3.77 (s, 1H), 3.65 - 3.39 (m, 4H), |
| | | 3.01 - 2.70 (m, 2H), 2.41 - 2.31 (m, 1H), 2.27 |
| | | (s, 4H), 2.18 - 1.96 (m, 1H), 1.87 - 1.66 (m, |
| | | 2H), 1.63 - 1.39 (m, 4H), 1.13 (d, J = 6.0 Hz, |
| | | 3H). |
| P-232 | o-{5-[(R)-4-(2-{3-[(R)-2-methyl-1- | ¹H NMR (400 MHz, d₄-Methanol) δ 8.11 (d, J |
| | pyrrolidinyl]-1-propynyl}-4- | = 6.0 Hz, 1H), 7.77 (d, J = 8.4 Hz, 1H), 7.52 |
| | pyrimidinyl)-5-methyl-1,4-diazepan- | (s, 1H), 7.30 - 7.22 (m, 1H), 6.98 - 6.88 (m, |
| | 1-yl]-6-amino-3-pyridazinyl }phenol | 2H), 6.76 (d, J = 6.4 Hz, 1H), 4.76 - 4.66 (m, |
| | | 1H), 4.18 - 4.02 (m, 1H), 3.95 - 3.87 (m, 1H), |
| | | 3.87 - 3.79 (m, 1H), 3.71 (d, J = 17.6 Hz, 1H), |
| | | 3.67 - 3.53 (m, 3H), 3.26 - 3.20 (m, 1H), 2.91 - |
| | | 2.75 (m, 3H), 2.44 - 2.32 (m, 1H), 2.29 - 2.17 |
| | | (m, 1H), 2.11 (dt, J = 7.2, 13.2 Hz, 1H), 1.92 - |
| | | 1.80 (m, 2H), 1.58 - 1.47 (m, 1H), 1.28 (s, 3H), |
| | | 1.22 (d, J = 6.4 Hz, 3H). |
| P-233 | o-{5-[(R)-4-(2-{3-[(S)-2-methyl-1- | ¹H NMR (400 MHz, d₄-Methanol) δ 8.10 (d, J |
| | pyrrolidinyl]-1-propynyl}-4- | = 6.4 Hz, 1H), 7.75 (d, J = 8.0 Hz, 1H), 7.50 |
| | pyrimidinyl)-5-methyl-1,4-diazepan- | (d, J = 1.6 Hz, 1H), 7.27 - 7.21 (m, 1H), 6.94 - |
| | 1-yl]-6-amino-3-pyridazinyl} phenol | 6.87 (m, 2H), 6.75 (d, J = 6.4 Hz, 1H), 4.72 - |
| | | 4.65 (m, 1H), 4.12 - 4.02 (m, 1H), 3.94 (d, J = |
| | | 17.6 Hz, 1H), 3.83 (dd, J = 8.8, 10.8 Hz, 1H), |
| | | 3.73 (d, J = 17.2 Hz, 1H), 3.65 - 3.51 (m, 3H), |
| | | 3.16 - 3.09 (m, 1H), 2.96 - 2.88 (m, 1H), 2.87 - |
| | | 2.79 (m, 2H), 2.41 - 2.33 (m, 1H), 2.17 (d, J = |
| | | 2.4 Hz, 1H), 2.11 (dd, J = 6.4, 13.2 Hz, 1H), |
| | | 1.91 - 1.82 (m, 2H), 1.56 - 1.49 (m, 1H), 1.30 - |
| | | 1.23 (m, 3H), 1.22 (d, J = 6.0 Hz, 3H). |
| P-234 | o-{5-[(R)-4-(2-{3-(2- | ¹H NMR (400 MHz, d₆-DMSO) δ 8.25 - 8.07 |
| | azabicyclo[2.1.1]hex-2-yl)-1- | (m, 2H), 7.98 - 7.81 (m, 1H), 7.54 (d, J = 7.6 |
| | propynyl}-4-pyrimidinyl)-5-methyl- | Hz, 1H), 7.28 - 7.16 (m, 1H), 6.94 - 6.79 (m, |
| | 1,4-diazepan-1-yl]-6-amino-3- | 2H), 6.74 (d, J = 6.4 Hz, 1H), 6.43 - 6.21 (m, |
| | pyridazinyl }phenol | 2H), 4.89 - 4.71 (m, 0.5H), 4.59 - 4.45 (m, |
| | | 0.5H), 4.09 - 3.89 (m, 1H), 3.76 (d, J = 9.6 Hz, |
| | | 2H), 3.15 - 2.92 (m, 3H), 2.91 - 2.81 (m, 1H), |
| | | 2.79 - 2.69 (m, 2H), 2.66 (s, 2H), 2.31 - 1.93 |
| | | (m, 3H), 1.65 (s, 2H), 1.44 - 1.39 (m, 2H), 1.13 |
| | | (d, J = 4.0 Hz, 3H). |
| P-235 | 5-[3-(4-{ (R)-4-[6-(o-hydroxyphenyl)- | ¹H NMR (400 MHz, d₄-Methanol) δ 8.88 (m, |
| | 4-pyridaziny1]-7-methyl-1,4- | 1H), 8.23 - 7.97 (m, 3H), 7.49 - 7.40 (m, 1H), |
| | diazepan-1-yl}-2-pyrimidinyl)-2- | 7.33 (t, J = 7.2 Hz, 2H), 6.97 - 6.84 (m, 3H), |
| | propynyl]-2,3-dimethyl-1,5,7a-triaza- | 6.78 (d, J = 5.6 Hz, 1H), 5.21 (s, 2H), 4.62 - |
| | 4(5H)-indenone | 3.47 (m, 7H), 2.29 - 2.13 (m, 1H), 1.76 (s, 1H), |
| | | 1.15 (d, J = 6.4 Hz, 3H) |
| P-236 | o-{5-[(R)-4-(2-{3-[(R)-3-fluoro-1- | ¹H NMR (400 MHz, d₄-Methanol) δ 8.09 (d, J |
| | pyrrolidinyl]-1-propynyl}-4- | = 6.0 Hz, 1H), 7.78 - 7.72 (m, 1H), 7.49 (s, |
| | pyrimidinyl)-5-methyl-1,4-diazepan- | 1H), 7.27 - 7.20 (m, 1H), 6.94 - 6.86 (m, 2H), |
| | 1-yl]-6-amino-3-pyridaziny1 } phenol | 6.73 (d, J = 6.4 Hz, 1H), 5.33 - 5.25 (m, 0.5H), |
| | | 5.18 - 5.12 (m, 0.5H), 4.72 - 4.58 (m, 1H), 4.15- |
| | | 3.99 (m, 1H), 3.83 (s, 1H), 3.71 (d, J = 1.2 |
| | | Hz, 2H), 3.65 - 3.47 (m, 3H), 3.13 - 3.01 (m, |
| | | 2H), 2.99 - 2.86 (m, 1H), 2.80 (t, J = 11.2 Hz, |
| | | 1H), 2.73 - 2.65 (m, 1H), 2.42 - 2.23 (m, 2H), |
| | | 2.23 - 1.97 (m, 2H), 1.29 - 1.21 (m, 3H) |
| P-237 | o-{5-[(R)-4-(2-{3-(6-aza-6- | ¹H NMR (400 MHz, d₄-Methanol) δ 8.09 (d, J |
| | spiro\| 3.4 \| octyl)-1-propynyl}-4- | = 6.0 Hz, 1H), 7.75 (d, J = 8.0 Hz, 1H), 7.49 |
| | pyrimidinyl)-5-methyl-1,4-diazepan- | (s, 1H), 7.28 - 7.19 (m, 1H), 6.95 - 6.86 (m, |
| | 1-yl]-6-amino-3-pyridazinyl } phenol | 2H), 6.73 (d, J = 6.4 Hz, 1H), 4.59 (s, 2H), |
| | | 4.14 - 4.00 (m, 1H), 3.83 (s, 1H), 3.65 - 3.48 |
| | | (m, 5H), 2.85 (s, 2H), 2.80 (t, J = 7.2 Hz, 2H), |
| | | 2.42 - 2.30 (m, 1H), 2.15 - 2.05 (m, 3H), 2.03 - |
| | | 1.92 (m, 4H), 1.91 - 1.79 (m, 2H), 1.25 (d, J = |
| | | 6.8 Hz, 3H) |
| P-238 | 4-\|(R)-4-{2-\|3-(tert- | m/z (ESI⁺) 531 (M + H)⁺. |
| | butoxycarbonylamino)-1-propynyl]- | |
| | 4-pyrimidinyl}-5-methyl-1,4- | |
| | diazepan-1-yl]-6-(o-hydroxyphenyl)- | |
| | 3-pyridazinamine | |
| P-239 | N-[3-(4-{ (R)-4-[3-amino-6-(o- | m/z (ESI⁺) 567 (M + H)⁺. |
| | hydroxyphenyl)-4-pyridazinyl]-7- | |
| | methyl-1,4-diazepan-1-y1}-2- | |
| | pyrimidinyl)-2-propynyl]-1-methyl- | |
| | 4,5-dimethyl-3-pyrazolecarboxamide | |
| P-240 | N-[3-(4-{ (R)-4-[3-amino-6-(o- | m/z (ESI⁺) 536 (M + H)⁺. |
| | hydroxyphenyl)-4-pyridazinyl]-7- | |
| | methyl-1,4-diazepan-1-yl}-2- | |
| | pyrimidinyl)-2- | |
| | propynyl]nicotinamide | |
| P-241 | N-[3-(4-{3-[3-amino-6-(o- | m/z (ESI⁺) 536 (M + H)⁺. |
| | hydroxyphenyl)-4-pyridazinyl]-3,8- | |
| | diazabicyclo[3.2.1]oct-8-yl}-2- | |
| | pyrimidinyl)-2- | |
| | propynyl]nicotinamide | |
| P-242 | o-{5-[(R)-4-(2-{3-[(R)-3-methoxy-1- | ¹H NMR (400 MHz, d₄-Methanol) δ 8.09 (d, J |
| | pyrrolidinyl]-1-propynyl}-4- | = 3.6 Hz, 1H), 7.74 (d, J = 7.6 Hz, 1H), 7.49 |
| | pyrimidinyl)-5-methyl-1,4-diazepan- | (d, J = 0.8 Hz, 1H), 7.24 (t, J = 7.6 Hz, 1H), |
| | 1-yl]-6-amino-3-pyridazinyl } phenol | 6.91 (d, J = 6.4 Hz, 2H), 6.74 (d, J = 6.0 Hz, |
| | | 1H), 4.68 (d, J = 12.0 Hz, 1H), 4.03 (s, 2H), |
| | | 3.82 (d, J = 5.6 Hz, 1H), 3.76 (s, 2H), 3.70 - |
| | | 3.44 (m, 4H), 3.26 - 3.13 (m, 1H), 3.13 - 2.88 |
| | | (m, 4H), 2.82 (d, J = 7.2 Hz, 2H), 2.42 - 2.30 |
| | | (m, 1H), 2.26 - 2.10 (m, 2H), 1.91 (d, J = 5.6 |
| | | Hz, 1H), 1.26 (s, 3H). |
| P-243 | o-{5-[(R)-4-{2-[3-(3,3-difluoro-1- | ¹H NMR (400 MHz, d₄-Methanol) δ 8.09 (d, J |
| | pyrrolidinyl)-1-propynyl]-4- | = 6.4 Hz, 1H), 7.76 (dd, J = 1.2, 8.0 Hz, 1H), |
| | pyrimidinyl }-5-methyl-1,4-diazepan- | 7.51 (d, J = 6.4 Hz, 1H), 7.27 - 7.21 (m, 1H), |
| | 1-yl]-6-amino-3-pyridazinyl} phenol | 6.94 - 6.86 (m, 2H), 6.74 (d, J = 6.4 Hz, 1H), |
| | | 4.76 - 4.67 (m, 1H), 4.14 - 4.00 (m, 1H), 3.89 - |
| | | 3.79 (m, 1H), 3.71 (s, 2H), 3.65 - 3.49 (m, 3H), |
| | | 3.12 (t, J = 13.2 Hz, 2H), 3.00 - 2.94 (m, 2H), |
| | | 2.88 - 2.78 (m, 1H), 2.41 - 2.15 (m, 4H), 1.26 |
| | | (s, 3H) |
| P-244 | o-{5-[(R)-4-(2-{3-[(S)-3-fluoro-1- | ¹H NMR (400 MHz, d₄-Methanol) δ 8.09 (d, J |
| | pyrrolidinyl]-1-propynyl}-4- | = 6.0 Hz, 1H), 7.74 (d, J = 7.2 Hz, 1H), 7.49 |
| | pyrimidinyl)-5-methyl-1,4-diazepan- | (s, 1H), 7.29 - 7.20 (m, 1H), 6.95 - 6.87 (m, |
| | 1-yl]-6-amino-3-pyridaziny1 }phenol | 2H), 6.73 (d, J = 6.4 Hz, 1H), 5.30 (t, J = 5.6 |
| | | Hz, 0.5H), 5.16 (dd, J = 4.8, 6.2 Hz, 0.5H), |
| | | 4.75 - 4.53 (m, 1H), 4.18 - 3.97 (m, 1H), 3.92 - |
| | | 3.79 (m, 1H), 3.73 (d, J = 1.6 Hz, 2H), 3.67 - |
| | | 3.46 (m, 3H), 3.17 - 3.04 (m, 2H), 3.04 - 2.88 |
| | | (m, 1H), 2.82 (dd, J = 9.2, 11.2 Hz, 1H), 2.76 - |
| | | 2.68 (m, 1H), 2.42 - 2.24 (m, 2H), 2.23 - 1.98 |
| | | (m, 2H), 1.25 (s, 3H). |
| P-245 | 1-[3-(4-{ (R)-4-[3-amino-6-(o- | ¹H NMR (400 MHz, d₄-Methanol) δ 8.10 (d, J |
| | hydroxyphenyl)-4-pyridazinyl \|-7- | = 6.0 Hz, 1H), 7.80 - 7.72 (m, 1H), 7.50 (s, |
| | methyl-1,4-diazepan-1-yl}-2- | 1H), 7.28 - 7.21 (m, 1H), 6.94 - 6.88 (m, 2H), |
| | pyrimidinyl)-2-propynyl]-3- | 6.74 (d, J = 6.4 Hz, 1H), 4.77 - 4.52 (m, 1H), |
| | pyrrolidinecarbonitrile | 4.15 - 3.99 (m, 1H), 3.83 (s, 1H), 3.74 (s, 2H), |
| | | 3.66 - 3.47 (m, 3H), 3.28 - 3.16 (m, 1H), 3.12 - |
| | | 3.06 (m, 1H), 3.03 - 2.91 (m, 2H), 2.85 - 2.77 |
| | | (m, 2H), 2.41 - 2.29 (m, 2H), 2.28 - 2.05 (m, |
| | | 2H), 1.26 (s, 3H). |
| P-246 | o-{5-[(R)-4-{2-[3-(2,5-dimethyl-1- | ¹H NMR (400 MHz, d₆-DMSO) δ 14.63 - |
| | pyrrolidinyl)-1-propynyl]-4- | 13.52 (m, 1H), 8.19 - 8.06 (m, 1H), 7.88 (s, |
| | pyrimidinyl}-5-methyl-1,4-diazepan- | 1H), 7.53 (d, J = 8.4 Hz, 1H), 7.28 - 7.17 (m, |
| | 1-yl]-6-amino-3-pyridazinyl }phenol | 1H), 6.92 - 6.80 (m, 2H), 6.73 (d, J = 6.4 Hz, |
| | | 1H), 6.40 - 6.18 (m, 2H), 4.79 (dd, J = 2.0, 6.8 |
| | | Hz, 0.5H), 4.51 (d, J = 14.4 Hz, 0.5H), 4.09 - |
| | | 3.93 (m, 0.5H), 3.76 (s, 0.5H), 3.70 (s, 2H), |
| | | 3.55 - 3.40 (m, 3H), 3.16 (d, J = 5.2 Hz, 1H), |
| | | 3.03 - 2.93 (m, 1H), 2.79 - 2.73 (m, 2H), 2.30 - |
| | | 2.17 (m, 1H), 2.08 - 1.94 (m, 1H), 1.91 - 1.71 |
| | | (m, 2H), 1.34 - 1.24 (m, 2H), 1.13 (s, 3H), 1.07- |
| | | 0.99 (m, 6H) |
| P-247 | o-{5-[(R)-4-{5-fluoro-2-[3-(1- | ¹H NMR (499 MHz, d₆-DMSO) δ 14.21 (s, |
| | imidazolyl)-1-propynyl]-4- | 1H), 8.22 (d, J = 7.1 Hz, 1H), 7.91 - 7.85 (m, |
| | pyrimidinyl }-5-methyl-1,4-diazepan- | 1H), 7.53 (s, 1H), 7.23 (td, J = 7.6, 1.5 Hz, |
| | 1-yl]-6-amino-3-pyridaziny1 }phenol | 1H), 6.87 (t, J = 7.6 Hz, 2H), 6.30 (s, 2H), 5.21 |
| | | (s, 2H), 4.57 (s, 1H), 4.16 (s, 1H), 3.67 (s, 1H), |
| | | 3.57 (d, J = 12.8 Hz, 1H), 3.47 (dd, J = 13.9, |
| | | 7.6 Hz, 1H), 3.05 (t, J = 11.8 Hz, 1H), 2.88 |
| | | (dd, J = 14.0, 9.4 Hz, 1H), 2.25 (dt, J = 14.7, |
| | | 7.1 Hz, 1H), 2.09 - 1.98 (m, 1H), 1.19 (d, J = |
| | | 6.2 Hz, 3H). |
| P-248 | 3-(4-{(R)-4-[3-amino-6-(o- | ¹H NMR (499 MHz, d₆-DMSO) δ 14.23 (s, |
| | hydroxyphenyl)-4-pyridazinyl\|-7- | 1H), 8.20 (d, J = 7.2 Hz, 1H), 7.91 - 7.86 (m, |
| | methyl-1,4-diazepan-1-yl}-5-fluoro- | 1H), 7.53 (s, 1H), 7.41 (q, J = 5.3 Hz, 1H), |
| | 2-pyrimidinyl)-2-propynylamino-tert- | 7.26 - 7.19 (m, 1H), 6.87 (t, J = 7.8 Hz, 2H), |
| | butylformylate | 6.31 (s, 2H), 4.58 (s, 1H), 4.17 (s, 1H), 3.97 (d, |
| | | J = 5.9 Hz, 2H), 3.68 (s, 1H), 3.56 (dd, J = |
| | | 12.9, 3.7 Hz, 1H), 3.47 (dd, J = 13.8, 7.7 Hz, |
| | | 1H), 3.06 (s, 1H), 2.89 (dd, J = 14.0, 9.7 Hz, |
| | | 1H), 2.25 (dt, J = 14.5, 6.9 Hz, 1H), 2.04 (q, J |
| | | = 11.4 Hz, 1H), 1.39 (s, 10H), 1.37 (d, J = 13.8 |
| | | Hz, 1H), 1.20 (d, J = 6.2 Hz, 3H). |
| P-249 | o-{5-[(R)-4-{2-[3-(2,4-dimethyl-1- | m/z (ESI⁺) 499 (M + H)⁺. |
| | azetidinyl)-1-propynyl]-4- | |
| | pyrimidinyl}-5-methyl-1,4-diazepan- | |
| | 1-yl]-6-amino-3-pyridaziny1 } phenol | |
| P-250 | o-{5-\|(S)-4-{2-\|3-(dimethylamino)-1- | m/z (ESI⁺) 459 (M + H)⁺. |
| | propynyl]-4-pyrimidinyl}-5-methyl- | |
| | 1,4-diazepan-1-yl]-6-amino-3- | |
| | pyridazinyl }phenol | |
| P-251 | o-{5-[(R)-4-[2-(3-{[3- | m/z (ESI⁺) 543 (M + H)⁺. |
| | (dimethylamino)cyclobutyl]methoxy}- | |
| | 1-propynyl)-4-pyrimidinyl]-5- | |
| | methyl-1,4-diazepan-1-yl]-6-amino-3- | |
| | pyridazinyl }phenol | |
| P-252 | o-{5-[(R)-4-{2-[3-(3,3-difluoro-1- | m/z (ESI⁺) 507 (M + H)⁺. |
| | azetidinyl)-1-propynyl]-4- | |
| | pyrimidinyl}-5-methyl-1,4-diazepan- | |
| | 1-yl]-6-amino-3-pyridazinyl}phenol | |
| P-253 | o-[6-amino-5-(8-{2-[3-(1- | m/z (ESI⁺) 508 (M + H)⁺. |
| | imidazolyl)-1-propynyl]-4- | |
| | pyrimidinyl}-2,8-diaza-2- | |
| | spiro [4.5]decyl)-3-pyridazinyl]phenol | |
| P-254 | o-{5-[(R)-4-{2-[3-(dimethylamino)-1- | ¹H NMR (499 MHz., d₆-DMSO) δ 14.13 (s, |
| | butynyl]-4-pyrimidinyl }-5-methyl- | 1H), 8.14 (s, 1H), 7.86 (d, J = 9.7 Hz, 1H), |
| | 1,4-diazepan-1-yl]-6-amino-3- | 7.54 (d, J = 8.5 Hz, 1H), 7.26 - 7.19 (m, 1H), |
| | pyridazinyl}phenol | 6.90 - 6.82 (m, 2H), 6.77 (s, 1H), 6.32 (d, J = |
| | | 22.5 Hz, 2H), 4.80 (s, 1H), 4.52 (d, J = 14.6 |
| | | Hz, 0H), 4.00 (s, 0H), 3.86 (s, 1H), 3.77 (s, |
| | | 1H), 3.50 (d, J = 15.0 Hz, 2H), 2.31 (s, 6H), |
| | | 2.04 (s, 1H), 1.36 - 1.31 (m, 3H), 1.13 (d, J = |
| | | 6.1 Hz, 3H). |
| P-255 | [3-(4-{4-[3-amino-6-(o- | ¹H NMR (400 MHz, d₆-DMSO) δ 14.22 (d, J = |
| | hydroxyphenyl)-4-pyridaziny1]-1,4- | 0.8 Hz, 1H), 8.12 (d, J = 5.6 Hz, 1H), 7.92 - |
| | diazepan-1-yl}-2-pyrimidinyl)-2- | 7.78 (m, 1H), 7.51 (s, 1H), 7.29 - 7.18 (m, 1H), |
| | propynylamino]acetonitrile | 6.93 - 6.83 (m, 2H), 6.73 (d, J = 6.4 Hz, 1H), |
| | | 6.17 (s, 2H), 4.11 - 3.74 (m, 4H), 3.69 (d, J = |
| | | 6.8 Hz, 2H), 3.63 (d, J = 6.0 Hz, 2H), 3.44 (d, J |
| | | = 3.2 Hz, 2H), 3.29 - 3.26 (m, 2H), 3.19 - 3.10 |
| | | (m, 1H), 2.05 (s, 2H). |
| P-256 | o-[6-amino-5-(4-{2-[2-(1-methyl-4- | ¹H NMR (400 MHz, d₄-Methanol) δ 8.04 (d, J |
| | piperidyl)ethynyl]-4-pyrimidinyl}- | = 6.4 Hz, 1H), 7.69 - 7.65 (m, 1H), 7.50 (s, |
| | 1,4-diazepan-1-yl)-3- | 1H), 7.29 - 7.20 (m, 1H), 6.96 - 6.86 (m, 2H), |
| | pyridazinyl]phenol | 6.67 (d, J = 6.4 Hz, 1H), 4.28 - 4.02 (m, 2H), |
| | | 3.91 - 3.69 (m, 2H), 3.55 (t, J = 4.8 Hz, 2H), |
| | | 3.39 (m, 2H), 2.86 - 2.66 (m, 3H), 2.36 - 2.23 |
| | | (m, 5H), 2.19 - 2.13 (m, 2H), 2.04 - 1.96 (m, |
| | | 2H), 1.87 - 1.77 (m, 2H) |
| P-257 | o-{5-[4-(2-{3-[(S)-1- | ¹H NMR (400 MHz, d₄-Methanol) δ 8.05 (d, J |
| | methylpropylamino]-1-propynyl}-4- | = 6.4 Hz, 1H), 7.68 - 7.61 (m, 2H), 7.59 - 7.53 |
| | pyrimidinyl)-1,4-diazepan-1-yl]-6- | (m, 1H), 7.50 - 7.47 (m, 1H), 7.26 - 7.21 (m, |
| | amino-3-pyridazinyl }phenol | 1H), 6.94 - 6.87 (m, 2H), 6.69 (d, J = 6.4 Hz, |
| | | 1H), 4.66 - 4.56 (m, 1H), 4.22 - 4.03 (m, 2H), |
| | | 3.80 - 3.74 (m, 1H), 3.65 (d, J = 5.2 Hz, 2H), |
| | | 3.55 (s, 2H), 3.39 (s, 2H), 2.89 (dd, J = 6.4, |
| | | 12.4 Hz, 1H), 2.14 (td, J = 6.0, 11.2 Hz, 2H), |
| | | 1.63 - 1.55 (m, 1H), 1.38 - 1.30 (m, 1H), 1.08 |
| | | (d, J = 6.4 Hz, 3H), 0.94 (t, J = 7.6 Hz, 3H). |
| P-258 | o-[6-amino-5-(4-{2-[3-(2- | ¹H NMR (400 MHz, d₄-Methanol) δ 8.10 - |
| | fluoroethylamino)-1-propynyl]-4- | 8.00 (m, 1H), 7.67 - 7.60 (m, 1H), 7.46 (s, 1H), |
| | pyrimidinyl}-1,4-diazepan-1-yl)-3- | 7.28 - 7.22 (m, 1H), 6.94 - 6.89 (m, 2H), 6.74 - |
| | pyridazinyl]phenol | 6.67 (m, 1H), 4.74 - 4.47 (m, 4H), 4.20 - 4.04 |
| | | (m, 2H), 3.80 (s, 2H), 3.63 - 3.58 (m, 2H), 3.46 |
| | | - 3.39 (m, 2H), 3.21 - 3.07 (m, 2H), 2.19 - 2.10 |
| | | (m, 2H) |
| P-259 | 3-(4-{7-[3-amino-6-(o- | ¹H NMR (400 MHz, d₆-DMSO) δ 14.79 - |
| | hydroxyphenyl)-4-pyridaziny1]-3,7- | 14.48 (m, 1H), 8.18 (d, J = 5.6 Hz, 1H), 7.94 |
| | diazabicyclo[3.3.0]oct-1(5)-en-3-y1}- | (d, J = 8.0 Hz, 1H), 7.46 - 7.38 (m, 1H), 7.23 |
| | 2-pyrimidinyl)-2-propynylamino-tert- | (t, J = 8.0 Hz, 1H), 7.07 (s, 1H), 6.92 - 6.81 (m, |
| | butylformylate | 2H), 6.56 (d, J = 6.0 Hz, 1H), 5.97 (s, 2H), |
| | | 4.57 (s, 4H), 4.37 - 4.25 (m, 4H), 3.99 (d, J = |
| | | 5.2 Hz, 2H), 1.41 (s, 9H) |
| P-260 | o-{5-[(R)-4-(2-{3-[(3R,5S)-3,5- | ¹H NMR (499 MHz, d₆-DMSO) δ 14.22 (s, |
| | dimethyl-4-morpholiny1]-1- | 1H), 8.13 (s, 1H), 7.87 (d, J = 10.3 Hz, 1H), |
| | propynyl}-4-pyrimidinyl)-5-methyl- | 7.54 (d, J = 12.6 Hz, 1H), 7.26 - 7.19 (m, 1H), |
| | 1,4-diazepan-1-yl]-6-amino-3- | 6.90 - 6.82 (m, 2H), 6.75 (d, J = 6.3 Hz, 1H), |
| | pyridaziny1 }phenol | 6.33 (s, 1H), 6.27 (s, 1H), 4.80 (s, 1H), 4.52 (d, |
| | | J = 14.5 Hz, 0H), 4.01 (s, 1H), 3.79 (d, J = 17.2 |
| | | Hz, 3H), 3.66 (dd, J = 11.0, 3.0 Hz, 2H), 3.51 |
| | | (s, 2H), 3.09 (t, J = 10.7 Hz, 2H), 2.98 (s, 1H), |
| | | 2.84 (s, 1H), 2.81 - 2.65 (m, 3H), 2.04 (s, 1H), |
| | | 1.13 (d, J = 6.2 Hz, 3H), 0.91 (d, J = 6.1 Hz, |
| | | 6H). |
| P-261 | 8-[3-(4-{(R)-4-[3-amino-6-(o- | ¹H NMR (499 MHz, d₆-DMSO) δ 8.13 (s, 2H), |
| | hydroxyphenyl)-4-pyridazinyl]-7- | 7.89 (dd, J = 19.4, 8.2 Hz, 1H), 7.54 (d, J = |
| | methyl-1,4-diazepan-1-yl}-2- | 13.5 Hz, 1H), 7.22 (ddd, J = 8.4, 7.1, 1.6 Hz, |
| | pyrimidinyl)-2-propynyl]-8- | 1H), 6.90 - 6.82 (m, 2H), 6.74 (d, J = 6.3 Hz, |
| | azabicyclo[3.2.1]octan-3-ol | 1H), 6.33 (s, 1H), 6.26 (s, 1H), 4.79 (s, 1H), |
| | | 4.52 (d, J = 14.6 Hz, 1H), 4.36 (s, 1H), 4.00 (s, |
| | | 1H), 3.82 (t, J = 5.0 Hz, 1H), 3.77 (s, 1H), 3.49 |
| | | (d, J = 15.5 Hz, 2H), 3.41 (s, 3H), 3.27 (s, 2H), |
| | | 3.01 (d, J = 12.0 Hz, 0H), 2.84 (s, 0H), 2.80 - |
| | | 2.71 (m, 1H), 2.23 (s, 1H), 2.08 (t, J = 6.5 Hz, |
| | | 2H), 1.94 - 1.76 (m, 4H), 1.62 (d, J = 13.9 Hz, |
| | | 2H), 1.12 (d, J = 6.2 Hz, 3H). |
| P-262 | o-{5-[(R)-4-(2-{3-[(R)-3-isopropyl-4- | m/z (ESI⁺) 543 (M + H)⁺. |
| | morpholinyl]-1-propynyl}-4- | |
| | pyrimidinyl)-5-methyl-1,4-diazepan- | |
| | 1-yl]-6-amino-3-pyridazinyl } phenol | |
| P-263 | o-{5-[(R)-4-(2-{3-[(S)-2- | ¹H NMR (499 MHz, d₆-DMSO) δ 8.16 (s, 1H), |
| | (fluoromethyl)-1-azetidinyl]-1- | 7.88 (d, J = 7.7 Hz, 1H), 7.54 (d, J = 16.9 Hz, |
| | propynyl}-4-pyrimidinyl)-5-methyl- | 1H), 7.26 - 7.19 (m, 1H), 6.90 - 6.82 (m, 2H), |
| | 1,4-diazepan-1-yl]-6-amino-3- | 6.77 (s, 1H), 6.55 (s, 1H), 6.33 (s, 1H), 6.27 (s, |
| | pyridazinyl }phenol | 1H), 4.80 (s, 1H), 4.42 (dddd, J = 48.0, 32.4, |
| | | 9.9, 4.2 Hz, 3H), 3.77 (s, 1H), 3.51 (s, 3H), |
| | | 3.21 (q, J = 6.1 Hz, 1H), 3.12 (d, J = 8.2 Hz, |
| | | 1H), 2.84 (s, 0H), 2.76 (dd, J = 14.0, 9.6 Hz, |
| | | 1H), 2.25 (s, 1H), 1.99 - 1.92 (m, 3H), 1.13 (s, |
| | | 4H). |
| P-264 | o-{5-[(R)-4-(2-{3-[(2R,4R)-2,4- | ¹H NMR (499 MHz, d₆-DMSO) δ 8.12 (s, 1H), |
| | dimethyl-1-azetidinyl]-1-propynyl}- | 7.91 - 7.82 (m, 1H), 7.53 (d, J = 19.0 Hz, 1H), |
| | 4-pyrimidinyl)-5-methyl-1,4- | 7.22 (ddd, J = 8.4, 7.3, 1.6 Hz, 1H), 6.90 - 6.82 |
| | diazepan-1-yl]-6-amino-3- | (m, 3H), 6.73 (d, J = 6.3 Hz, 1H), 6.40 - 6.20 |
| | pyridazinyl}phenol | (m, 2H), 4.78 (s, 1H), 4.51 (d, J = 14.4 Hz, |
| | | 1H), 3.99 (s, 1H), 3.76 (s, 1H), 3.60 (h, J = 6.4 |
| | | Hz, 2H), 3.05 (s, 1H), 2.84 (d, J = 11.2 Hz, |
| | | 0H), 2.76 (dd, J = 13.9, 9.5 Hz, 1H), 2.25 - |
| | | 2.20 (m, 1H), 2.03 (d, J = 12.6 Hz, 1H), 1.79 (t, |
| | | J = 6.4 Hz, 2H), 1.21 (d, J = 6.3 Hz, 6H), 1.16 |
| | | - 1.07 (m, 3H). |
| P-265 | o-{5-[(R)-4-{2-[(S)-3-cyclopropyl-3- | ¹H NMR (499 MHz, d₆-DMSO) δ 14.22 (s, |
| | hydroxy-1-propynyl]-4-pyrimidinyl}- | 1H), 8.13 (s, 1H), 7.97 - 7.84 (m, 1H), 7.54 (d, |
| | 5-methyl-1,4-diazepan-1-yl]-6-amino- | J = 11.8 Hz, 1H), 7.26 - 7.19 (m, 1H), 6.86 |
| | 3-pyridaziny1 } phenol | (dd, J = 10.5, 7.8 Hz, 2H), 6.74 (d, J = 6.3 Hz, |
| | | 1H), 6.30 (d, J = 32.5 Hz, 2H), 5.60 (s, 1H), |
| | | 4.12 (d, J = 6.9 Hz, 1H), 4.00 (s, 1H), 3.77 (s, |
| | | 1H), 3.51 (s, 1H), 3.00 (s, 1H), 2.89 (s, 1H), |
| | | 2.76 (t, J = 11.3 Hz, 1H), 2.73 (s, 1H), 2.23 (d, |
| | | J = 10.8 Hz, 1H), 2.14 (s, 0H), 2.02 (d, J = 13.5 |
| | | Hz, 1H), 1.18 (q, J = 5.8 Hz, 1H), 1.13 (d, J = |
| | | 6.0 Hz, 3H), 0.49 (dq, J = 9.6, 3.4 Hz, 2H), |
| | | 0.39 (dq, J = 5.5, 3.5 Hz, 2H). |
| P-266 | 0-{5-[(R)-4-{2-[(R)-3-cyclopropyl-3- | ¹H NMR (499 MHz, d₆-DMSO) δ 14.22 (s, |
| | hydroxy-1-propynyl]-4-pyrimidinyl}- | 1H), 8.13 (s, 1H), 7.93 - 7.84 (m, 1H), 7.54 (d, |
| | 5-methyl-1,4-diazepan-1-yl]-6-amino- | J = 12.6 Hz, 1H), 7.22 (td, J = 7.7, 1.6 Hz, 1H), |
| | 3-pyridaziny1 } phenol | 6.86 (td, J = 8.2, 2.1 Hz, 2H), 6.74 (d, J = 6.2 |
| | | Hz, 1H), 6.33 (s, 1H), 6.27 (s, 1H), 5.61 (s, |
| | | 1H), 4.12 (d, J = 6.9 Hz, 1H), 4.00 (s, 1H), |
| | | 3.77 (s, 1H), 3.49 (d, J = 16.5 Hz, 2H), 3.00 (s, |
| | | 0H), 2.84 (s, 1H), 2.76 (t, J = 11.7 Hz, 1H), |
| | | 2.24 (s, 1H), 2.14 (s, 1H), 2.03 (d, J = 13.2 Hz, |
| | | 1H), 1.18 (q, J = 5.8 Hz, 1H), 1.13 (d, J = 6.2 |
| | | Hz, 3H), 0.48 (dt, J = 8.4, 3.0 Hz, 2H), 0.39 |
| | | (dt, J = 5.2, 2.8 Hz, 2H). |
| P-267 | 3-(4-{9-[3-amino-6-(o- | m/z (ESI⁺) 543 (M + H)⁺. |
| | hydroxyphenyl)-4-pyridaziny1]-3,9- | |
| | diazabicyclo[4.2.1]non-3-yl}-2- | |
| | pyrimidinyl)-2-propynylamino-tert- | |
| | butylformylate | |
| P-268 | 4-[(R)-4-(2-{3-[(Z)-4-cycloocten-1- | ¹H NMR (499 MHz, d₆-DMSO) δ 8.13 (dd, J = |
| | yloxycarbonylamino]-1-propynyl}-4- | 13.1, 6.3 Hz, 1H), 7.92 (d, J = 7.9 Hz, 1H), |
| | pyrimidinyl)-5-methyl-1,4-diazepan- | 7.86 (d, J = 7.9 Hz, 1H), 7.61 - 7.51 (m, 2H), |
| | 1-yl]-6-(o-hydroxyphenyl)-3- | 7.26 - 7.19 (m, 1H), 6.90 - 6.83 (m, 2H), 6.74 |
| | pyridazinamine | (d, J = 6.4 Hz, 1H), 6.33 (s, 1H), 6.26 (s, 1H), |
| | | 5.57 (s, 2H), 5.44 (t, J = 13.6 Hz, 1H), 4.80 (s, |
| | | 1H), 4.51 (d, J = 14.8 Hz, 1H), 4.26 (d, J = 6.6 |
| | | Hz, 1H), 4.00 (t, J = 5.5 Hz, 3H), 3.77 (s, 1H), |
| | | 3.48 (s, 1H), 3.03 (s, 1H), 2.84 (s, 1H), 2.75 |
| | | (dd, J = 13.7, 9.3 Hz, 1H), 2.27 (s, 5H), 2.13 (s, |
| | | 1H), 2.02 (d, J = 12.1 Hz, 1H), 1.93 (s, 2H), |
| | | 1.85 (s, 2H), 1.65 (d, J = 12.5 Hz, 1H), 1.58 (s, |
| | | 3H), 1.13 (d, J = 7.3 Hz, 4H). |
| P-269 | 0-{5-[(R)-4-{2-[2-(3,3- | m/z (ESI⁺) 492 (M + H)⁺. |
| | difluorocyclobutyl)ethynyl]-4- | |
| | pyrimidinyl }-5-methyl-1,4-diazepan- | |
| | 1-y1]-6-amino-3-pyridazinyl } phenol | |
| P-270 | 0-{5-[(R)-4-(2-{3-[(S)-3,3-difluoro-2- | ¹H NMR (499 MHz, d₆-DMSO) δ 14.24 (s, |
| | methyl-1-azetidinyl]-1-propyny1}-4- | 1H), 8.16 (d, J = 8.2 Hz, 1H), 7.87 (s, 1H), |
| | pyrimidinyl)-5-methyl-1,4-diazepan- | 7.56 (s, 0H), 7.51 (s, 0H), 7.26 - 7.19 (m, 1H), |
| | 1-yl]-6-amino-3-pyridaziny1 } phenol | 6.90 - 6.82 (m, 1H), 6.76 (d, J = 6.3 Hz, 1H), |
| | | 6.30 (d, J = 33.9 Hz, 2H), 4.78 (s, 0H), 4.51 (d, |
| | | J = 14.6 Hz, 0H), 4.01 (s, 0H), 3.77 (s, 1H), |
| | | 3.80 - 3.71 (m, 1H), 3.74 - 3.58 (m, 3H), 3.60 |
| | | - 3.46 (m, 2H), 3.50 (s, 1H), 2.99 (s, 0H), 2.85 |
| | | (s, 0H), 2.76 (dd, J = 13.9, 9.4 Hz, 1H), 2.28 |
| | | 2.20 (m, 1H), 1.20 - 1.05 (m, 7H). |
| P-271 | 0-{5-[(R)-4-(2-{3-[(R)-3,3-difluoro- | ¹H NMR (499 MHz, d₆-DMSO) δ 14.21 (s, |
| | 2-methyl-1-azetidinyl]-1-propynyl}- | 1H), 8.14 (q, J = 6.2 Hz, 1H), 7.87 (d, J = 7.9 |
| | 4-pyrimidinyl)-5-methyl-1,4- | Hz, 1H), 7.53 (d, J = 23.4 Hz, 1H), 7.26 - 7.19 |
| | diazepan-1-yl]-6-amino-3- | (m, 1H), 6.90 - 6.82 (m, 2H), 6.76 (d, J = 6.4 |
| | pyridazinyl }phenol | Hz, 1H), 6.30 (d, J = 33.0 Hz, 2H), 4.01 (s, |
| | | 1H), 3.82 - 3.63 (m, 5H), 3.59 - 3.52 (m, 0H), |
| | | 3.54 - 3.47 (m, 1H), 3.00 (d, J = 12.6 Hz, 0H), |
| | | 2.84 (s, 1H), 2.81 - 2.72 (m, 1H), 2.25 (dt, J = |
| | | 14.5, 6.9 Hz, 1H), 2.13 (d, J = 10.3 Hz, 1H), |
| | | 2.04 (d, J = 12.1 Hz, 1H), 1.15 (dd, J = 27.8, |
| | | 7.0 Hz, 6H). |
| P-272 | 0-[6-amino-5-(4-{2-[3-(3- | ¹H NMR (400 MHz, d₄-Methanol) δ 8.04 (d, J |
| | oxetanylamino)-1-propynyl]-4- | = 6.0 Hz, 1H), 7.69 - 7.62 (m, 1H), 7.48 (s, |
| | pyrimidinyl}-1,4-diazepan-1-yl)-3- | 1H), 7.27 - 7.19 (m, 1H), 6.95 - 6.87 (m, 2H), |
| | pyridazinyl]phenol | 6.68 (d, J = 6.4 Hz, 1H), 4.63 (t, J = 6.4 Hz, |
| | | 2H), 4.58 (s, 2H), 4.21 - 4.10 (m, 2H), 3.83 - |
| | | 3.70 (m, 2H), 3.66 (s, 1H), 3.61 (s, 2H), 3.55 |
| | | (s, 2H), 3.39 (s, 2H), 2.14 (quin, J = 6.0 Hz, |
| | | 2H). |
| P-273 | 0-{5-[4-(2-{3-[(R)-tetrahydro-3- | ¹H NMR (400 MHz, d₄-Methanol) δ 8.06 (d, J |
| | furylamino]-1-propynyl }-4- | = 6.4 Hz, 1H), 7.66 (dd, J = 1.6, 8.0 Hz, 1H), |
| | pyrimidinyl)-1,4-diazepan-1-yl]-6- | 7.50 (s, 1H), 7.25 (dt, J = 1.6, 7.6 Hz, 1H), |
| | amino-3-pyridazinyl}phenol | 6.95 - 6.89 (m, 2H), 6.70 (d, J = 6.4 Hz, 1H), |
| | | 4.27 - 4.07 (m, 2H), 3.95 - 3.89 (m, 3H), 3.79 |
| | | (dt, J = 6.4, 8.0 Hz, 3H), 3.66 (s, 2H), 3.65 - |
| | | 3.63 (m, 1H), 3.57 (d, J = 5.2 Hz, 2H), 3.41 (s, |
| | | 2H), 2.19 - 2.13 (m, 3H), 1.88 - 1.78 (m, 1H). |
| P-274 | 0-{5-[4-(2-{3-[(R)-1- | ¹H NMR (400 MHz, d₄-Methanol) δ 8.06 (d, J |
| | methylpropylamino]-1-propynyl }-4- | = 6.4 Hz, 1H), 7.66 (dd, J = 1.6, 8.0 Hz, 1H), |
| | pyrimidinyl)-1,4-diazepan-1-yl]-6- | 7.50 (s, 1H), 7.29 - 7.21 (m, 1H), 6.97 - 6.86 |
| | amino-3-pyridazinyl}phenol | (m, 2H), 6.70 (d, J = 6.4 Hz, 1H), 4.72 - 4.51 |
| | | (m, 1H), 4.25 - 4.00 (m, 2H), 3.84 - 3.76 (m, |
| | | 1H), 3.67 (d, J = 5.2 Hz, 2H), 3.56 (s, 2H), |
| | | 3.41 (s, 2H), 2.90 (dd, J = 6.4, 12.4 Hz, 1H), |
| | | 2.23 - 2.07 (m, 2H), 1.67 - 1.53 (m, 1H), 1.40 - |
| | | 1.29 (m, 1H), 1.10 (d, J = 6.4 Hz, 3H), 0.96 (t, |
| | | J = 7.6 Hz, 3H). |
| P-275 | 0-{5-[4-(2-{3-[(S)-tetrahydro-3- | ¹H NMR (400 MHz, d₄-Methanol) δ 8.05 (d, J |
| | furylamino]-1-propynyl }-4- | = 6.4 Hz, 1H), 7.65 (dd, J = 1.6, 8.4 Hz, 1H), |
| | pyrimidinyl)-1,4-diazepan-1-yl]-6- | 7.48 (s, 1H), 7.28 - 7.19 (m, 1H), 6.95 - 6.86 |
| | amino-3-pyridaziny1}phenol | (m, 2H), 6.69 (d, J = 6.4 Hz, 1H), 4.28 - 4.05 |
| | | (m, 1H), 4.24 - 4.05 (m, 1H), 3.96 - 3.87 (m, |
| | | 3H), 3.78 (dt, J = 6.0, 8.0 Hz, 3H), 3.69 - 3.66 |
| | | (m, 1H), 3.64 (s, 2H), 3.56 (d, J = 4.8 Hz, 2H), |
| | | 3.40 (s, 2H), 2.21 - 2.11 (m, 3H), 1.86 - 1.77 |
| | | (m, 1H). |
| P-276 | 0-{5-[4-(2-{3-[(S)-2-methoxy-1- | ¹H NMR (400 MHz, d₄-Methanol) δ 8.04 (d, J |
| | methylethylamino]-1-propynyl }-4- | = 6.4 Hz, 1H), 7.65 (dd, J = 1.2, 8.4 Hz, 1H), |
| | pyrimidinyl)-1,4-diazepan-1-yl]-6- | 7.52 - 7.45 (m, 1H), 7.28 - 7.19 (m, 1H), 6.96 - |
| | amino-3-pyridazinyl}phenol | 6.85 (m, 2H), 6.69 (d, J = 6.4 Hz, 1H), 4.24 - |
| | | 3.90 (m, 3H), 3.76 (s, 1H), 3.72 (s, 1H), 3.67 |
| | | (s, 1H), 3.57 - 3.52 (m, 2H), 3.43 - 3.37 (m, |
| | | 4H), 3.36 (s, 3H), 3.21 (dt, J = 4.4, 6.8 Hz, |
| | | 1H), 2.20 - 2.07 (m, 2H), 1.07 (d, J = 6.4 Hz, |
| | | 3H |
| P-277 | 0-{5-[4-(2-{3-[(R)-2-methoxy-1- | ¹H NMR (400 MHz, d₄-Methanol) δ 8.04 (d, J |
| | methylethylamino]-1-propynyl}-4- | = 6.4 Hz, 1H), 7.65 (dd, J = 1.6, 8.4 Hz, 1H), |
| | pyrimidinyl)-1,4-diazepan-1-yl]-6- | 7.53 - 7.44 (m, 1H), 7.29 - 7.19 (m, 1H), 6.97 - |
| | amino-3-pyridazinyl}phenol | 6.84 (m, 2H), 6.69 (d, J = 6.4 Hz, 1H), 4.33 - |
| | | 3.94 (m, 3H), 3.76 (s, 1H), 3.72 (s, 1H), 3.67 |
| | | (s, 1H), 3.54 (d, J = 4.8 Hz, 2H), 3.43 - 3.37 |
| | | (m, 4H), 3.36 (s, 3H), 3.21 (dq, J = 2.0, 6.4 Hz, |
| | | 1H), 2.18 - 2.08 (m, 2H), 1.07 (d, J = 6.4 Hz, |
| | | 3H). |
| P-278 | 0-[6-amino-5-(4-{2-[3-(3-methyl-3- | ¹H NMR (400 MHz, d₄-Methanol) 8.04 (d, J = |
| | oxetanylamino)-1-propynyl]-4- | 6.4 Hz, 1H), 7.65 (dd, J = 1.6, 8.4 Hz, 1H), |
| | pyrimidinyl}-1,4-diazepan-1-yl)-3- | 7.48 (s, 1H), 7.29 - 7.19 (m, 1H), 6.94 - 6.86 |
| | pyridazinyl]phenol | (m, 2H), 6.69 (d, J = 6.0 Hz, 1H), 4.77 (d, J = |
| | | 6.4 Hz, 2H), 4.43 (d, J = 6.8 Hz, 2H), 4.21 - |
| | | 4.06 (m, 1H), 3.85 - 3.70 (m, 2H), 3.66 (s, 2H), |
| | | 3.55 (s, 2H), 3.39 (s, 3H), 2.19 - 2.10 (m, 2H), |
| | | 1.58 (s, 3H). |
| P-279 | 0-{5-[4-(2-{3-[(S)-2-methyl-1- | m/z (ESI⁺) 471 (M + H)⁺. |
| | azetidinyl]-1-propynyl }-4- | |
| | pyrimidinyl)-1,4-diazepan-1-y1]-6- | |
| | amino-3-pyridazinyl}phenol | |
| P-280 | 0-(6-amino-5-{4-[2-(3-piperidino-1- | ¹H NMR (499 MHz, d₆-DMSO) δ 14.16 (s, |
| | propynyl)-4-pyrimidinyl]-1- | 1H), 8.18 (d, J = 6.2 Hz, 1H), 7.94 - 7.89 (m, |
| | piperazinyl}-3-pyridazinyl)phenol | 1H), 7.56 (s, 1H), 7.24 (t, J = 7.6 Hz, 1H), 6.88 |
| | | (dd, J = 8.4, 6.1 Hz, 3H), 6.44 (s, 2H), 3.88 - |
| | | 3.83 (m, 4H), 3.46 (s, 2H), 3.15 (t, J = 5.0 Hz, |
| | | 4H), 2.46 (d, J = 5.7 Hz, 4H), 1.53 (p, J = 5.6 |
| | | Hz, 4H), 1.38 (q, J = 6.1 Hz, 2H). |
| P-281 | 5-[3-(4-{4-[3-amino-6-(o- | m/z (ESI⁺) 549 (M + H)⁺. |
| | hydroxyphenyl)-4-pyridazinyl]-1- | |
| | piperazinyl}-2-pyrimidinyl)-2- | |
| | propynyl]-2,3-dimethyl-1,5,7a-triaza- | |
| | 4(5H)-indenone | |
| P-282 | 5-[3-(4-{ 8-[3-amino-6-(o- | m/z (ESI⁺) 575 (M + H)⁺. |
| | hydroxyphenyl)-4-pyridaziny1]-3,8- | |
| | diazabicyclo[3.2.1]oct-3-yl}-2- | |
| | pyrimidinyl)-2-propynyl]-2,3- | |
| | dimethyl-1,5,7a-triaza-4(5H)- | |
| | indenone | |
| P-283 | 0-[6-amino-5-(3-{2-[3-(1H-1,2,4- | ¹H NMR (499 MHz, d₆-DMSO) δ 8.65 (s, 1H), |
| | triazol-1-yl)-1-propynyl]-4- | 8.18 (d, J = 6.1 Hz, 1H), 8.08 (s, 1H), 7.97 (dd, |
| | pyrimidinyl}-3,8- | J = 8.0, 1.6 Hz, 1H), 7.46 (s, 1H), 7.24 (td, J = |
| | diazabicyclo[3.2.1]oct-8-yl)-3- | 7.6, 1.6 Hz, 1H), 6.89 (t, J = 8.3 Hz, 2H), 6.82 |
| | pyridazinyl]phenol | (d, J = 6.3 Hz, 1H), 6.20 (s, 2H), 5.44 (s, 2H), |
| | | 4.46 (d, J = 4.5 Hz, 2H), 1.97 (d, J = 9.7 Hz, |
| | | 2H), 1.67 (d, J = 7.8 Hz, 2H). |
| P-284 | 0-[6-amino-5-(3-{2-[3-(1- | ¹H NMR (499 MHz, d₆-DMSO) δ 8.18 (d, J = |
| | imidazolyl)-1-propynyl]-4- | 6.3 Hz, 1H), 7.97 (dd, J = 8.0, 1.6 Hz, 1H), |
| | pyrimidinyl}-3,8- | 7.46 (s, 1H), 7.24 (td, J = 7.6, 1.6 Hz, 1H), 7.00 |
| | diazabicyclo[3.2.1]oct-8-yl)-3- | - 6.96 (m, 1H), 6.93 - 6.85 (m, 3H), 6.82 (d, J |
| | pyridazinyl]phenol | = 6.3 Hz, 1H), 6.20 (s, 3H), 5.21 (s, 2H), 4.46 |
| | | (d, J = 4.0 Hz, 2H), 3.38 - 3.30 (m, 4H), 2.00 - |
| | | 1.93 (m, 2H), 1.66 (q, J = 6.4 Hz, 2H). |
| P-285 | 0-(5-{(1R,4R)-5-[2-(3-piperidino-1- | ¹H NMR (499 MHz, d₆-DMSO) δ 8.14 (s, 1H), |
| | propynyl)-4-pyrimidinyl]-2,5- | 8.06 (d, J = 7.0 Hz, 1H), 7.98 (dd, J = 15.1, 7.9 |
| | diazabicyclo[2.2.1]hept-2-y1}-6- | Hz, 1H), 7.23 (t, J = 7.3 Hz, 2H), 6.91 - 6.83 |
| | amino-3-pyridazinyl)phenol | (m, 2H), 5.92 (s, 2H), 5.09 (s, 1H), 4.98 (s, |
| | | 1H), 4.87 - 4.83 (m, 1H), 3.95 (dd, J = 18.1, |
| | | 9.4 Hz, 1H), 3.71 (d, J = 11.5 Hz, 0H), 3.55 |
| | | (dd, J = 20.3, 9.1 Hz, 1H), 3.46 (s, 1H), 3.40 (s, |
| | | 1H), 3.36 (s, 0H), 2.47 (s, 2H), 2.41 (s, 2H), |
| | | 2.10 (d, J = 10.3 Hz, 0H), 2.04 (d, J = 12.5 Hz, |
| | | 2H), 1.50 (dt, J = 28.8, 5.8 Hz, 5H), 1.37 (s, |
| | | 1H), 1.32 (s, 1H). |
| P-286 | 5-[3-(4-{(1R,4R)-5-[3-amino-6-(0- | m/z (ESI⁺) 561 (M + H)⁺. |
| | hydroxyphenyl)-4-pyridazinyl]-2,5- | |
| | diazabicyclo[2.2.1]hept-2-y1}-2- | |
| | pyrimidinyl)-2-propynyl]-2,3- | |
| | dimethyl-1,5,7a-triaza-4(5H)- | |
| | indenone | |
| P-287 | 0-[6-amino-5-(8-{2-[3-(1-methyl-3- | m/z (ESI⁺) 509 (M + H)⁺. |
| | pyrazolylamino)-1-propynyl\|-4- | |
| | pyrimidinyl}-3,8- | |
| | diazabicyclo[3.2.1]oct-3-yl)-3- | |
| | pyridazinyl]phenol | |
| P-288 | 0-(6-amino-5-{8-[2-(3-piperidino-1- | m/z (ESI⁺) 497 (M + H)⁺. |
| | propynyl)-4-pyrimidinyl]-3,8- | |
| | diazabicyclo[3.2.1]oct-3-y1}-3- | |
| | pyridazinyl)phenol | |
| P-289 | 0-[6-amino-5-(8-{2-[3-(1H-1,2,4- | ¹H NMR (499 MHz, d₆-DMSO) δ 7.97 (d, J = |
| | triazol-1-yl)-1-propynyl]-4- | 7.8 Hz, 1H), 7.47 (s, 1H), 7.28 - 7.21 (m, 1H), |
| | pyrimidinyl}-3,8- | 6.89 (t, J = 8.0 Hz, 2H), 6.82 (s, 1H), 6.53 (s, |
| | diazabicyclo[3.2.1]oct-3-yl)-3- | 1H), 6.23 - 6.19 (m, 2H), 4.48 (d, J = 4.7 Hz, |
| | pyridazinyl]phenol | 2H), 4.04 (s, 3H), 3.51 (s, 2H), 2.01 - 1.95 (m, |
| | | 2H), 1.67 (q, J = 6.7 Hz, 2H), 1.54 (q, J = 5.6 |
| | | Hz, 4H), 1.38 (s, 2H). |
| P-290 | 0-[6-amino-5-(8-{2-[3-(1- | m/z (ESI⁺) 480 (M + H)⁺. |
| | imidazolyl)-1-propynyl\|-4- | |
| | pyrimidinyl}-3,8- | |
| | diazabicyclo[3.2.1]oct-3-yl)-3- | |
| | pyridazinyl]phenol | |
| P-291 | 0-[6-amino-5-(8-{2-[3- | ¹H NMR (499 MHz, d₆-DMSO) δ 8.20 - 8.13 |
| | (methylamino)-1-propynyl]-4- | (m, 1H), 8.00 - 7.90 (m, 1H), 7.47 (s, 1H), |
| | pyrimidinyl}-3,8- | 7.24 (td, J = 7.6, 1.6 Hz, 1H), 6.89 (t, J = 8.1 |
| | diazabicyclo[3.2.1]oct-3-yl)-3- | Hz, 3H), 6.83 - 6.76 (m, 1H), 6.21 (s, 3H), |
| | pyridazinyl]phenol | 4.50 - 4.43 (m, 3H), 3.53 (s, 2H), 3.37 - 3.33 |
| | | (m, 1H), 2.35 (s, 3H), 2.01 - 1.95 (m, 3H), |
| | | 1.67 (q, J = 6.3 Hz, 2H). |
| P-292 | 0-{5-[(S)-2-methyl-4-{2-[3- | ¹H NMR (499 MHz, d₆-DMSO) δ 8.16 (dd, J = |
| | (methylamino)-1-propynyl]-4- | 6.4, 3.8 Hz, 1H), 7.92 (dd, J = 8.0, 1.6 Hz, 1H), |
| | pyrimidinyl}-1-pipcrazinyl]-6-amino- | 7.69 (s, 1H), 7.27 - 7.20 (m, 1H), 6.96 - 6.83 |
| | 3-pyridaziny1} phenol | (m, 3H), 6.49 (s, 2H), 4.03 (d, J = 12.8 Hz, |
| | | 2H), 3.84 - 3.77 (m, 1H), 3.73 - 3.48 (m, 1H), |
| | | 2.83 (dt, J = 15.9, 5.5 Hz, 1H), 2.36 (d, J = 8.6 |
| | | Hz, 2H), 0.90 (d, J = 6.3 Hz, 3H). |
| P-293 | 0-{5-[(S)-4-{2-[3-(1-imidazolyl)-1- | ¹H NMR (499 MHz, d₆-DMSO) δ 14.09 (s, |
| | propynyl]-4-pyrimidinyl}-2-methyl- | 1H), 8.17 (d, J = 6.4 Hz, 1H), 7.92 (dd, J = 8.0, |
| | 1-piperaziny1]-6-amino-3- | 1.7 Hz, 1H), 7.80 - 7.75 (m, 1H), 7.68 (s, 1H), |
| | pyridazinyl }phenol | 7.30 (s, 1H), 7.23 (ddd, J = 8.5, 7.2, 1.6 Hz, |
| | | 1H), 6.97 (s, 1H), 6.94 (d, J = 6.4 Hz, 1H), |
| | | 6.91 - 6.84 (m, 2H), 6.51 - 6.42 (m, 2H), 5.21 |
| | | (s, 2H), 4.02 (d, J = 13.0 Hz, 2H), 3.84 - 3.77 |
| | | (m, 1H), 3.71 - 3.63 (m, 1H), 3.63 - 3.55 (m, |
| | | 1H), 2.85 - 2.77 (m, 1H), 0.89 (d, J = 6.3 Hz, |
| | | 3H). |
| P-294 | 0-[6-amino-5-(4-{2-[3- | m/z (ESI⁺) 417 (M + H)⁺. |
| | (methylamino)-1-propynyl]-4- | |
| | pyrimidinyl}-1-piperazinyl)-3- | |
| | pyridazinyl]phenol | |
| P-295 | 0-{5-[(1R,4R)-5-{2-[3- | m/z (ESI⁺) 429 (M + H)⁺. |
| | (methylamino)-1-propynyl]-4- | |
| | pyrimidinyl}-2,5- | |
| | diazabicyclo[2.2.1]hept-2-y1]-6- | |
| | amino-3-pyridazinyl } phenol | |
| P-296 | 4-(1-{2-[3-(tert- | ¹H NMR (400 MHz, d₆-DMSO) δ 14.41 (s, |
| | butoxycarbonylamino)-1-propynyl]- | 1H), 8.16 (d, J = 6.4 Hz, 1H), 8.00 (d, J = 7.6 |
| | 4-pyrimidinyl }-4-piperidyloxy)-6-(o- | Hz, 1H), 7.71 (s, 1H), 7.43 (t, J = 4.8 Hz, 1H), |
| | hydroxyphenyl)-3-pyridazinamine | 7.31 - 7.24 (m, 1H), 6.95 - 6.88 (m, 3H), 6.60 |
| | | (s, 2H), 5.23 (d, J = 2.8 Hz, 1H), 3.99 (d, J = |
| | | 6.0 Hz, 4H), 3.68 (m, 2H), 2.07 - 1.97 (m, 2H), |
| | | 1.83 - 1.72 (m, 2H), 1.42 (s, 9H) |
| P-297 | 0-{5-[(R)-5-methyl-4-{2-[3- | ¹H NMR (499 MHz, d₆-DMSO) δ 8.13 (s, 1H), |
| | (methylamino)-1-propynyl]-4- | 7.88 (s, 2H), 7.54 (d, J = 13.0 Hz, 1H), 7.22 (t, |
| | pyrimidinyl}-1,4-diazepan-1-yl]-6- | J = 7.6 Hz, 1H), 6.90 - 6.83 (m, 2H), 6.73 (d, J |
| | amino-3-pyridazinyl } phenol | = 6.3 Hz, 1H), 6.32 (s, 2H), 6.26 (s, 1H), 4.80 |
| | | (s, 1H), 4.52 (s, 0H), 4.00 (s, 1H), 3.76 (s, 1H), |
| | | 3.51 (s, 2H), 2.76 (t, J = 12.1 Hz, 1H), 2.34 (s, |
| | | 3H), 2.24 (s, 2H), 2.11 (s, 2H), 2.03 (s, 1H), |
| | | 1.13 (s, 4H). |
| P-298 | 0-{5-[(R)-4-{2-[3-(1-imidazolyl)-1- | ¹H NMR (499 MHz, d₆-DMSO) δ 14.24 (s, |
| | propynyl]-4-pyrimidinyl}-5-mcthyl- | 1H), 8.14 (s, 1H), 7.94 - 7.82 (m, 1H), 7.54 (d, |
| | 1,4-diazepan-1-yl]-6-amino-3- | J = 12.0 Hz, 1H), 7.26 - 7.19 (m, 1H), 6.87 (t, |
| | pyridazinyl }phenol | J = 9.3 Hz, 2H), 6.78 (d, J = 6.3 Hz, 1H), 6.29 |
| | | (d, J = 36.0 Hz, 2H), 5.22 (s, 2H), 4.78 (s, 1H), |
| | | 4.51 (d, J = 14.7 Hz, 0H), 4.01 (s, 0H), 3.77 (s, |
| | | 1H), 3.49 (d, J = 15.1 Hz, 1H), 3.00 (s, 0H), |
| | | 2.84 (s, 0H), 2.75 (dd, J = 14.0, 9.5 Hz, 1H), |
| | | 2.24 (s, 1H), 2.09 (d, J = 60.6 Hz, 1H), 1.12 (d, |
| | | J = 7.8 Hz, 3H). |
| P-299 | 0-{5-[4-(2-{3-[(R)-2-methyl-1- | m/z (ESI⁺) 471 (M + H)⁺. |
| | azetidinyl]-1-propynyl }-4- | |
| | pyrimidinyl)-1,4-diazepan-1-y1]-6- | |
| | amino-3-pyridaziny1} phenol | |
| P-300 | 0-[6-amino-5-(4-{2-[3-(tetrahydro- | ¹H NMR (400 MHz, d₄-Methanol) δ 8.05 (d, J |
| | 2H-pyran-4-ylamino)-1-propynyl]-4- | = 5.2 Hz, 1H), 7.63 (d, J = 7.6 Hz, 1H), 7.47 |
| | pyrimidinyl}-1,4-diazepan-1-yl)-3- | (s, 1H), 7.24 (t, J = 7.6 Hz, 1H), 6.95 - 6.86 (m, |
| | pyridazinyl]phenol | 2H), 6.70 (d, J = 6.4 Hz, 1H), 4.22 - 4.02 (m, |
| | | 2H), 3.96 (dd, J = 2.4, 11.6 Hz, 2H), 3.73 (s, |
| | | 4H), 3.57 (s, 2H), 3.50 - 3.39 (m, 4H), 3.12 - |
| | | 3.01 (m, 1H), 2.21 - 2.08 (m, 2H), 1.91 (dd, J = |
| | | 1.8, 12.4 Hz, 2H), 1.44 (dq, J = 4.8, 12.0 Hz, |
| | | 2H). |
| P-301 | 0-[6-amino-5-(4-{2-[3- | ¹H NMR (400 MHz, d₄-Methanol) δ 8.05 (d, J |
| | (cyclopropylamino)-1-propynyl]-4- | = 6.4 Hz, 1H), 7.70 - 7.60 (m, 1H), 7.51 - 7.44 |
| | pyrimidinyl}-1,4-diazepan-1-yl)-3- | (m, 1H), 7.28 - 7.17 (m, 1H), 6.95 - 6.83 (m, |
| | pyridazinyl]phenol | 2H), 6.69 (d, J = 6.0 Hz, 1H), 4.22 - 3.99 (m, |
| | | 2H), 3.87 - 3.70 (m, 2H), 3.65 (s, 2H), 3.56 (d, |
| | | J = 5.6 Hz, 2H), 3.40 (s, 2H), 2.45 (tt, J = 3.6, |
| | | 6.8 Hz, 1H), 2.18 - 2.10 (m, 2H), 0.55 - 0.40 |
| | | (m, 4H) |
| P-302 | N-[3-(4-{4-[3-amino-6-(o- | ¹H NMR (400 MHz, d₆-DMSO) δ 13.90 (s, |
| | hydroxyphenyl)-4-pyridazinyl]-1- | 1H), 9.13 (d, J = 5.2 Hz, 2H), 8.65 (d, J = 2.4 |
| | azepanyl}-2-pyrimidinyl)-2- | Hz, 1H), 8.11 (d, J = 5.2 Hz, 1H), 7.94 - 7.85 |
| | propynyl]-1,4,7a-triaza-2- | (m, 2H), 7.25 - 7.16 (m, 2H), 7.12 (s, 1H), 6.90 |
| | indenecarboxamide | - 6.81 (m, 2H), 6.71 - 6.62 (m, 3H), 4.32 (d, J |
| | | = 5.2 Hz, 2H), 3.92 - 3.55 (m, 2H), 3.44 (s, |
| | | 1H), 2.88 (t, J = 8.8 Hz, 1H), 2.11 - 1.93 (m, |
| | | 2H), 1.92 - 1.57 (m, 4H). |
| P-303 | 4-(9-{2-[3-(tert- | m/z (ESI⁺) 543 (M + H)⁺. |
| | butoxycarbonylamino)-1-propynyl]- | |
| | 4-pyrimidinyl}-3,9- | |
| | diazabicyclo[4.2.1]non-3-yl)-6-(o- | |
| | hydroxyphenyl)-3-pyridazinamine | |
| P-304 | 4-(5-{2-[3-(tert- | ¹H NMR (499 MHz, d₆-DMSO) δ 13.82 (s, |
| | butoxycarbonylamino)-1-propynyl]- | 1H), 8.03 (s, 1H), 7.76 (d, J = 7.9 Hz, 1H), |
| | 4-pyrimidinyl }-1,5-diazocan-1-yl)-6- | 7.39 (d, J = 9.6 Hz, 2H), 7.27 - 7.20 (m, 1H), |
| | (o-hydroxyphenyl)-3-pyridazinamine | 6.93 - 6.85 (m, 2H), 6.58 (d, J = 5.4 Hz, 1H), |
| | | 6.02 (s, 2H), 3.96 (d, J = 5.8 Hz, 2H), 3.78 (s, |
| | | 2H), 3.62 (s, 2H), 3.54 (s, 4H), 1.98 (s, 2H), |
| | | 1.88 (s, 2H), 1.40 (s, 9H). |
| P-305 | 0-{5-[4-(2-{3-[(3S,5S)-3,5-dimethyl- | m/z (ESI⁺) 515 (M + H)⁺. |
| | 4-morpholinyl]-1-propynyl }-4- | |
| | pyrimidinyl)-1,4-diazepan-1-yl]-6- | |
| | amino-3-pyridazinyl}phenol | |
| P-306 | 0-[5-(4-{2-[2-(1,3a-diaza-4- | ¹H NMR (499 MHz, d₆-DMSO) δ 14.16 (s, |
| | indenyl)ethynyl]-4-pyrimidinyl}-1,4- | 1H), 8.25 (s, 1H), 8.14 (s, 1H), 7.83 (d, J = 7.8 |
| | diazepan-1-yl)-6-amino-3- | Hz, 1H), 7.51 (s, 1H), 7.44 (d, J = 7.0 Hz, 1H), |
| | pyridazinyl]phenol | 7.34 (s, 1H), 7.21 (t, J = 7.7 Hz, 1H), 6.89 - |
| | | 6.81 (m, 3H), 6.19 (s, 2H), 4.10 (s, 1H), 3.96 |
| | | (s, 1H), 3.81 (s, 1H), 3.71 (s, 1H), 3.50 (s, 2H), |
| | | 2.09 (s, 2H). |
| P-307 | 4-(3-{2-[3-(tert- | ¹H NMR (400 MHz, d₄-Methanol) δ 8.01 (d, J |
| | butoxycarbonylamino)-1-propynyl]- | = 6.0 Hz, 1H), 7.80 - 7.74 (m, 1H), 7.25 (t, J = |
| | 4-pyrimidinylamino }-1-azetidinyl)-6- | 7.2 Hz, 1H), 6.97 (s, 1H), 6.94 - 6.88 (m, 2H), |
| | (o-hydroxyphenyl)-3-pyridazinamine | 6.50 (d, J = 6.0 Hz, 1H), 4.62 (t, J = 8.0 Hz, |
| | | 3H), 4.08 - 4.01 (m, 4H), 1.46 (s, 9H). |
| P-308 | 0-[6-amino-5-(4-{2-[3-(1-azetidinyl)- | m/z (ESI⁺) 457 (M + H)⁺. |
| | 1-propynyl]-4-pyrimidinyl}-1,4- | |
| | diazepan-1-yl)-3-pyridazinyl]phenol | |
| P-309 | 0-[6-amino-5-(4-{2-[3-(3-methoxy-1- | m/z (ESI⁺) 487 (M + H)⁺. |
| | azetidinyl)-1-propynyl]-4- | |
| | pyrimidinyl}-1,4-diazepan-1-yl)-3- | |
| | pyridazinyl]phenol | |
| P-310 | 4-[3-(4-{4-[3-amino-6-(o- | m/z (ESI⁺) 512 (M + H)⁺. |
| | hydroxyphenyl)-4-pyridazinyl]-1,4- | |
| | diazepan-1-y1}-2-pyrimidinyl)-2- | |
| | propynyl]-2-morpholinecarbonitrile | |
| P-311 | 0-[6-amino-5-(4-{2-[3-(2,2-dimethyl- | m/z (ESI⁺) 515 (M + H)⁺. |
| | 4-morpholinyl)-1-propynyl]-4- | |
| | pyrimidinyl}-1,4-diazepan-1-yl)-3- | |
| | pyridazinyl]phenol | |
| P-312 | 0-[6-amino-5-(4-{2-[3-(3,3-difluoro- | m/z (ESI⁺) 493 (M + H)⁺. |
| | 1-azetidinyl)-1-propynyl]-4- | |
| | pyrimidinyl}-1,4-diazepan-1-yl)-3- | |
| | pyridazinyl]phenol | |
| P-313 | 0-{5-[4-(2-{3-[(R)-2-methyl-4- | m/z (ESI⁺) 501 (M + H)⁺. |
| | morpholinyl]-1-propynyl}-4- | |
| | pyrimidinyl)-1,4-diazepan-1-yl]-6- | |
| | amino-3-pyridaziny1}phenol | |
| P-314 | tert-butyl (S)-2-[2-(4-{4-[3-amino-6- | m/z (ESI⁺) 557 (M + H)⁺. |
| | (o-hydroxyphenyl)-4-pyridazinyl]- | |
| | 1,4-diazepan-1-yl}-2- | |
| | pyrimidinyl)ethynyl]-1- | |
| | pyrrolidinecarboxylate | |
| P-315 | 0-{6-amino-5-[4-(2-{3-(8-oxa-3- | m/z (ESI⁺) 513 (M + H)⁺. |
| | azabicyclo[3.2.1]oct-3-yl)-1- | |
| | propynyl}-4-pyrimidinyl)-1,4- | |
| | diazepan-1-yl]-3-pyridazinyl } phenol | |
| P-316 | 0-{5-[4-(2-{3-[(S)-3-methyl-4- | m/z (ESI⁺) 501 (M + H)⁺. |
| | morpholinyl]-1-propynyl}-4- | |
| | pyrimidinyl)-1,4-diazepan-1-yl]-6- | |
| | amino-3-pyridazinyl}phenol | |
| P-317 | 0-[6-amino-5-(4-{2-[3-(3,3-dimethyl- | m/z (ESI⁺) 515 (M + H)⁺. |
| | 4-morpholinyl)-1-propynyl]-4- | |
| | pyrimidinyl}-1,4-diazepan-1-yl)-3- | |
| | pyridazinyl]phenol | |
| P-318 | 0-{5-[4-(2-{3-[(3R,5S)-3,5-dimethyl- | m/z (ESI⁺) 515 (M + H)⁺. |
| | 4-morpholinyl]-1-propynyl }-4- | |
| | pyrimidinyl)-1,4-diazepan-1-yl]-6- | |
| | amino-3-pyridazinyl }phenol | |
| P-319 | 0-(5-{(S)-2-methyl-4-[2-(3- | ¹H NMR (499 MHz, d₆-DMSO) δ 14.10 (s, |
| | piperidino-1-propynyl)-4- | 1H), 8.19 - 8.12 (m, 1H), 7.92 (dd, J = 8.0, 1.7 |
| | pyrimidinyl]-1-piperazinyl}-6-amino- | Hz, 1H), 7.69 (s, 1H), 7.23 (ddd, J = 8.5, 7.1, |
| | 3-pyridazinyl)phenol | 1.6 Hz, 1H), 6.93 - 6.84 (m, 3H), 6.49 (s, 2H), |
| | | 4.03 (d, J = 13.1 Hz, 2H), 3.85 - 3.78 (m, 1H), |
| | | 3.67 (t, J = 9.5 Hz, 1H), 3.59 (dd, J = 13.1, 5.8 |
| | | Hz, 1H), 3.47 (s, 2H), 3.39 (td, J = 7.7, 3.7 Hz, |
| | | 1H), 2.82 (ddd, J = 12.6, 6.6, 3.4 Hz, 1H), 2.47 |
| | | (s, 4H), 1.53 (p, J = 5.6 Hz, 4H), 1.38 (s, 2H), |
| | | 0.90 (d, J = 6.3 Hz, 3H). |
| P-320 | 5-[3-(4-{(S)-4-[3-amino-6-(o- | ¹H NMR (499 MHz, d₆-DMSO) δ 14.11 (s, |
| | hydroxyphenyl)-4-pyridazinyl]-3- | 1H), 8.16 (d, J = 6.3 Hz, 1H), 7.91 (dd, J = 8.1, |
| | methyl-1-piperaziny1}-2- | 1.6 Hz, 1H), 7.67 (d, J = 6.4 Hz, 2H), 7.27 - |
| | pyrimidinyl)-2-propynyl]-2,3- | 7.20 (m, 1H), 7.09 (d, J = 5.9 Hz, 1H), 6.93 (d, |
| | dimethyl-1,5,7a-triaza-4(5H)- | J = 6.3 Hz, 1H), 6.91 - 6.84 (m, 2H), 6.49 (s, |
| | indenone | 2H), 4.95 (s, 2H), 4.03 (s, 2H), 3.80 (td, J = |
| | | 6.3, 3.4 Hz, 1H), 3.67 (s, 1H), 3.58 (dd, J = |
| | | 13.2, 5.8 Hz, 1H), 2.80 (ddt, J = 12.1, 6.2, 3.6 |
| | | Hz, 1H), 2.34 (s, 3H), 2.25 (s, 3H), 0.88 (d, J = |
| | | 6.3 Hz, 3H). |
| P-321 | 0-[5-(4-{2-[(S)-3-hydroxy-1- | m/z (ESI⁺) 432 (M + H)⁺. |
| | butynyl]-4-pyrimidinyl}-1,4- | |
| | diazepan-1-yl)-6-amino-3- | |
| | pyridazinyl]phenol | |
| P-322 | 0-[5-(4-{2-[(R)-3-hydroxy-1- | ¹H NMR (499 MHz, d₆-DMSO) δ 14.21 (s, |
| | butynyl]-4-pyrimidinyl}-1,4- | 1H), 8.12 (d, J = 6.3 Hz, 1H), 7.85 (d, J = 7.8 |
| | diazepan-1-yl)-6-amino-3- | Hz, 1H), 7.51 (s, 1H), 7.23 (t, J = 7.6 Hz, 1H), |
| | pyridazinyl]phenol | 6.87 (dt, J = 7.4, 3.3 Hz, 2H), 6.72 (d, J = 6.3 |
| | | Hz, 1H), 6.17 (s, 2H), 5.58 (s, 1H), 4.58 (q, J = |
| | | 6.6 Hz, 1H), 4.03 (s, 1H), 3.88 (s, 1H), 3.77 (s, |
| | | 2H), 3.66 (s, 1H), 3.43 (t, J = 4.9 Hz, 2H), 3.27 |
| | | (t, J = 5.6 Hz, 2H), 2.05 (p, J = 5.9 Hz, 2H), |
| | | 1.38 (d, J = 6.6 Hz, 3H). |
| P-323 | 0-[6-amino-5-(4-{2-[3-(methylthio)- | ¹H NMR (499 MHz, d₆-DMSO) δ 14.25 (s, |
| | 1-propynyl]-4-pyrimidinyl}-1,4- | 1H), 8.12 (s, 1H), 7.83 (s, 1H), 7.51 (s, 1H), |
| | diazepan-1-yl)-3-pyridazinyl]phenol | 7.26 - 7.19 (m, 1H), 6.87 (ddd, J = 7.4, 4.0, 2.7 |
| | | Hz, 2H), 6.72 (d, J = 6.3 Hz, 1H), 6.17 (s, 2H), |
| | | 4.04 (s, 1H), 3.87 (s, 2H), 3.76 (s, 1H), 3.68 - |
| | | 3.64 (m, 1H), 3.60 (s, 2H), 3.44 (t, J = 5.2 Hz, |
| | | 2H), 3.27 (t, J = 5.7 Hz, 2H), 2.21 (s, 3H), 2.05 |
| | | (dd, J = 7.4, 4.3 Hz, 2H). |
| P-324 | 0-[6-amino-5-(4-{2-[3-(3- | ¹H NMR (400 MHz, d₄-Methanol) δ 8.03 (d, J |
| | cyclopropylureido)-1-propynyl]-4- | = 6.4 Hz, 1H), 7.67 (d, J = 8.0 Hz, 1H), 7.50 |
| | pyrimidinyl}-1,4-diazepan-1-yl)-3- | (s, 1H), 7.24 (dt, J = 1.6, 7.6 Hz, 1H), 6.96 - |
| | pyridazinyl]phenol | 6.86 (m, 2H), 6.68 (d, J = 6.0 Hz, 1H), 4.18 (s, |
| | | 2H), 4.15 - 3.63 (m, 4H), 3.54 (s, 2H), 3.39 (s, |
| | | 1H), 2.48 (tt, J = 3.6, 6.8 Hz, 1H), 2.19 - 2.03 |
| | | (m, 2H), 0.78 - 0.67 (m, 2H), 0.55 - 0.42 (m, |
| | | 2H). |
| P-325 | 0-[6-amino-5-(4-{2-[3-(3- | ¹H NMR (400 MHz, d₄-Methanol) δ 8.03 (d, J |
| | cyclobutylureido)-1-propynyl]-4- | = 2.8 Hz, 1H), 7.66 (d, J = 7.6 Hz, 1H), 7.49 |
| | pyrimidinyl}-1,4-diazepan-1-yl)-3- | (s, 1H), 7.24 (t, J = 8.4 Hz, 1H), 6.96 - 6.84 (m, |
| | pyridazinyl]phenol | 2H), 6.68 (d, J = 6.4 Hz, 1H), 6.44 - 6.30 (m, |
| | | 1H), 4.22 - 4.09 (m, 4H), 4.02 - 3.65 (m, 3H), |
| | | 3.54 (s, 2H), 3.39 (d, J = 1.6 Hz, 2H), 2.33 - |
| | | 2.22 (m, 2H), 2.13 (s, 2H), 1.94 - 1.81 (m, 2H), |
| | | 1.73 - 1.62 (m, 2H). |
| P-326 | 0-[6-amino-5-(4-{2-[3-(3- | ¹H NMR (400 MHz, d₄-Methanol) δ 8.08 - |
| | ethylureido)-1-propynyl]-4- | 7.98 (m, 1H), 7.71 - 7.64 (m, 1H), 7.50 (s, 1H), |
| | pyrimidinyl}-1,4-diazepan-1-yl)-3- | 7.24 (t, J = 6.8 Hz, 1H), 6.96 - 6.87 (m, 2H), |
| | pyridazinyl]phenol | 6.74 - 6.63 (m, 1H), 4.17 - 4.14 (m, 2H), 4.14 - |
| | | 3.63 (m, 3H), 3.59 - 3.49 (m, 2H), 3.38 (d, J = |
| | | 3.2 Hz, 2H), 3.22 - 3.11 (m, 3H), 2.18 - 2.08 |
| | | (m, 2H), 1.11 (t, J = 7.2 Hz, 3H). |
| P-327 | 0-[6-amino-5-(4-{2-[3-(3- | ¹H NMR (400 MHz, d₄-Methanol) δ 8.03 (d, J |
| | isopropylureido)-1-propynyl]-4- | = 4.4 Hz, 1H), 7.66 (d, J = 7.6 Hz, 1H), 7.49 |
| | pyrimidinyl}-1,4-diazepan-1-yl)-3- | (s, 1H), 7.30 - 7.18 (m, 1H), 6.95 - 6.85 (m, |
| | pyridazinyl]phenol | 2H), 6.68 (d, J = 6.0 Hz, 1H), 4.58 (s, 2H), |
| | | 4.15 (s, 2H), 3.90 - 3.73 (m, 3H), 3.54 (s, 2H), |
| | | 3.39 (s, 2H), 2.18 - 2.08 (m, 2H), 1.13 (d, J = |
| | | 6.4 Hz, 6H). |
| P-328 | 0-{5-[(S)-2-methyl-4-{2-[3-(1- | ¹H NMR (499 MHz, d₆-DMSO) δ 14.11 (s, |
| | methyl-3-pyrazolylamino)-1- | 1H), 8.15 (d, J = 6.3 Hz, 1H), 7.92 (dd, J = 8.1, |
| | propynyl]-4-pyrimidinyl}-1- | 1.7 Hz, 1H), 7.69 (s, 1H), 7.35 (d, J = 2.2 Hz, |
| | piperazinyl]-6-amino-3- | 1H), 7.24 (ddd, J = 8.5, 7.1, 1.6 Hz, 1H), 6.92 - |
| | pyridazinyl }phenol | 6.84 (m, 3H), 6.50 (s, 2H), 5.64 (t, J = 6.5 Hz, |
| | | 1H), 5.51 (d, J = 2.2 Hz, 1H), 4.04 (d, J = 6.4 |
| | | Hz, 2H), 3.83 - 3.77 (m, 1H), 3.68 (s, 1H), |
| | | 3.63 (s, 3H), 3.58 (dd, J = 13.3, 6.1 Hz, 1H), |
| | | 2.84 - 2.77 (m, 1H), 0.89 (d, J = 6.3 Hz, 3H). |
| P-329 | 0-{5-[(S)-2-methyl-4-{2-[3-(1- | ¹H NMR (499 MHz, d₆-DMSO) δ 14.11 (s, |
| | pyrazolyl)-1-propynyl]-4- | 1H), 8.18 (d, J = 6.3 Hz, 1H), 7.92 (dd, J = 8.1, |
| | pyrimidinyl }-1-piperazinyl]-6-amino- | 1.6 Hz, 1H), 7.85 (d, J = 2.3 Hz, 1H), 7.69 (s, |
| | 3-pyridazinyl } phenol | 1H), 7.53 (d, J = 1.9 Hz, 1H), 7.24 (ddd, J = |
| | | 8.5, 7.2, 1.6 Hz, 1H), 6.94 (d, J = 6.4 Hz, 1H), |
| | | 6.92 - 6.84 (m, 2H), 6.50 (s, 2H), 6.33 (t, J = |
| | | 2.1 Hz, 1H), 5.33 (s, 2H), 4.03 (d, J = 12.5 Hz, |
| | | 2H), 3.84 - 3.77 (m, 1H), 3.68 (s, 1H), 3.59 |
| | | (dd, J = 13.2, 5.9 Hz, 1H), 3.37 (s, 1H), 2.81 |
| | | (ddd, J = 12.1, 6.4, 3.2 Hz, 1H), 0.89 (d, J = 6.3 |
| | | Hz, 3H). |
| P-330 | 0-{5-[(S)-2-methyl-4-{2-[3-(1H- | ¹H NMR (499 MHz, d₆-DMSO) δ 14.10 (s, |
| | 1,2,4-triazol-1-yl)-1-propynyl]-4- | 1H), 8.64 (s, 1H), 8.17 (d, J = 6.4 Hz, 1H), |
| | pyrimidinyl}-1-piperazinyl]-6-amino- | 8.07 (s, 1H), 7.91 (dd, J = 8.0, 1.6 Hz, 1H), |
| | 3-pyridazinyl } phenol | 7.68 (s, 1H), 7.23 (ddd, J = 8.4, 7.1, 1.6 Hz, |
| | | 1H), 6.94 (d, J = 6.3 Hz, 1H), 6.91 - 6.83 (m, |
| | | 2H), 6.49 (s, 2H), 5.44 (s, 2H), 4.02 (d, J = |
| | | 11.2 Hz, 2H), 3.80 (td, J = 6.3, 3.4 Hz, 1H), |
| | | 3.71 - 3.64 (m, 1H), 3.59 (dd, J = 13.3, 5.9 Hz, |
| | | 1H), 3.41 - 3.33 (m, 2H), 2.80 (ddd, J = 12.4, |
| | | 6.5, 3.2 Hz, 1H), 0.88 (d, J = 6.2 Hz, 3H). |
| P-331 | 0-{5-[(R)-5-methyl-4-{2-[3-(1- | ¹H NMR (499 MHz, d₆-DMSO) δ 8.11 (s, 1H), |
| | methyl-3-pyrazolylamino)-1- | 7.53 (d, J = 13.5 Hz, 1H), 7.35 (s, 1H), 7.26 - |
| | propynyl]-4-pyrimidinyl }-1,4- | 7.19 (m, 1H), 6.90 - 6.83 (m, 2H), 6.72 (d, J = |
| | diazepan-1-yl]-6-amino-3- | 6.3 Hz, 1H), 6.60 (s, 1H), 6.33 (s, 1H), 6.26 (s, |
| | pyridazinyl}phenol | 1H), 5.63 (s, 1H), 5.51 (s, 1H), 4.04 (d, J = 6.4 |
| | | Hz, 2H), 3.62 (s, 3H), 2.75 (dd, J = 14.4, 9.5 |
| | | Hz, 1H), 2.24 (s, 2H), 1.24 (s, 1H), 1.12 (s, |
| | | 4H), 0.07 (s, 1H). |
| P-332 | 0-{5-[(R)-5-methyl-4-{2-[3-(1- | ¹H NMR (499 MHz, d₆-DMSO) δ 14.24 (d, J = |
| | pyrazolyl)-1-propynyl]-4- | 20.4 Hz, 1H), 8.14 (d, J = 10.2 Hz, 1H), 7.92 |
| | pyrimidinyl}-1,4-diazepan-1-y1]-6- | (d, J = 7.8 Hz, 0H), 7.85 (d, J = 2.3 Hz, 1H), |
| | amino-3-pyridazinyl}phenol | 7.56 - 7.50 (m, 2H), 7.22 (ddd, J = 8.5, 7.2, 1.6 |
| | | Hz, 1H), 6.90 - 6.82 (m, 2H), 6.77 (d, J = 6.3 |
| | | Hz, 1H), 6.32 (t, J = 2.4 Hz, 2H), 6.26 (s, 1H), |
| | | 5.32 (s, 2H), 4.79 (s, 1H), 4.51 (d, J = 14.3 Hz, |
| | | 1H), 4.01 (s, 1H), 3.77 (s, 1H), 3.48 (s, 2H), |
| | | 3.02 (s, 1H), 2.83 (s, 1H), 2.74 (dd, J = 14.0, |
| | | 9.5 Hz, 1H), 2.23 (s, 1H), 2.15 (s, 1H), 2.02 (s, |
| | | 1H), 1.12 (s, 3H). |
| P-333 | 0-{5-[(R)-5-methyl-4-{2-[3-(1H- | ¹H NMR (499 MHz, d₆-DMSO) δ 14.23 (s, |
| | 1,2,4-triazol-1-yl)-1-propynyl]-4- | 1H), 8.64 (s, 1H), 8.14 (s, 1H), 8.07 (s, 1H), |
| | pyrimidinyl}-1,4-diazepan-1-y1]-6- | 7.89 (dd, J = 25.7, 8.0 Hz, 1H), 7.53 (d, J = |
| | amino-3-pyridazinyl } phenol | 12.8 Hz, 1H), 7.26 - 7.19 (m, 1H), 6.86 (dd, J |
| | | = 10.6, 7.5 Hz, 2H), 6.78 (d, J = 6.4 Hz, 1H), |
| | | 6.33 (s, 1H), 6.26 (s, 1H), 5.44 (s, 2H), 4.78 (s, |
| | | 1H), 4.51 (d, J = 14.6 Hz, 1H), 4.01 (s, 1H), |
| | | 3.77 (s, 1H), 3.51 (s, 2H), 3.02 (d, J = 11.9 Hz, |
| | | 1H), 2.83 (s, 1H), 2.74 (dd, J = 14.0, 9.5 Hz, |
| | | 1H), 2.02 (d, J = 13.0 Hz, 1H), 1.12 (t, J = 8.0 |
| | | Hz, 3H). |
| P-334 | 0-(5-{(R)-5-methyl-4-[2-(3- | ¹H NMR (499 MHz, d₆-DMSO) δ 14.23 (s, |
| | morpholino-1-propynyl)-4- | 1H), 8.13 (s, 1H), 7.88 (d, J = 15.1 Hz, 1H), |
| | pyrimidinyl]-1,4-diazepan-1-yl}-6- | 7.54 (d, J = 12.2 Hz, 1H), 7.22 (ddd, J = 8.5, |
| | amino-3-pyridazinyl)phenol | 7.1, 1.6 Hz, 1H), 6.90 - 6.82 (m, 2H), 6.75 (d, |
| | | J = 6.3 Hz, 1H), 6.30 (d, J = 33.0 Hz, 2H), 4.80 |
| | | (s, 1H), 4.53 (d, J = 14.3 Hz, 1H), 4.01 (s, 1H), |
| | | 3.77 (s, 1H), 3.61 (s, 5H), 3.51 (s, 4H), 2.99 (s, |
| | | 1H), 2.84 (s, 1H), 2.76 (dd, J = 13.9, 9.6 Hz, |
| | | 1H), 2.24 (s, 1H), 2.04 (s, 1H), 1.13 (s, 4H). |
| P-335 | 0-(5-{(R)-5-methyl-4-[2-(3- | ¹H NMR (499 MHz, d₆-DMSO) δ 7.86 (s, 1H), |
| | piperidino-1-propynyl)-4- | 7.53 (d, J = 12.7 Hz, 1H), 7.23 (t, J = 7.8 Hz, |
| | pyrimidinyl]-1,4-diazepan-1-y1}-6- | 1H), 6.87 (dd, J = 13.7, 7.6 Hz, 2H), 6.80 (s, |
| | amino-3-pyridazinyl)phenol | 1H), 6.53 (s, 0H), 6.33 (d, J = 24.7 Hz, 2H), |
| | | 4.02 (s, 1H), 3.78 (s, 1H), 3.51 (d, J = 9.1 Hz, |
| | | 2H), 3.00 (s, 1H), 2.86 (s, 1H), 2.77 (d, J = |
| | | 12.6 Hz, 1H), 2.15 (s, 1H), 2.05 (s, 1H), 1.64 |
| | | (s, 4H), 1.45 (s, 2H), 1.13 (d, J = 8.0 Hz, 3H). |
| P-336 | 5-[3-(4-{ (R)-4-[3-amino-6-(o- | ¹H NMR (499 MHz, d₆-DMSO) δ 14.23 (s, |
| | hydroxyphenyl)-4-pyridazinyl]-7- | 1H), 8.13 (s, 1H), 7.88 (d, J = 21.1 Hz, 1H), |
| | methyl-1,4-diazepan-1-yl}-2- | 7.66 (d, J = 5.9 Hz, 1H), 7.53 (d, J = 10.8 Hz, |
| | pyrimidinyl)-2-propynyl]-2,3- | 1H), 7.22 (t, J = 7.6 Hz, 1H), 7.09 (s, 1H), 6.86 |
| | dimethyl-1,5,7a-triaza-4(5H)- | (dd, J = 13.0, 7.8 Hz, 2H), 6.77 (d, J = 6.0 Hz, |
| | indenone | 1H), 6.29 (d, J = 36.6 Hz, 2H), 4.94 (s, 1H), |
| | | 4.79 (s, 0H), 4.67 (d, J = 2.6 Hz, 1H), 4.00 (s, |
| | | 1H), 3.77 (s, 1H), 3.02 (s, 1H), 2.83 (s, 0H), |
| | | 2.74 (dd, J = 13.9, 9.5 Hz, 1H), 2.32 (d, J = 9.0 |
| | | Hz, 3H), 2.25 (d, J = 4.5 Hz, 4H), 2.16 (s, 6H), |
| | | 1.23 (s, 1H), 1.12 (s, 3H). |
| P-337 | 0-[6-amino-5-(4-{2-[3-(4-benzyl-1- | m/z (ESI⁺) 575 (M + H)⁺. |
| | piperidyl)-1-propynyl]-4- | |
| | pyrimidinyl}-1,4-diazepan-1-yl)-3- | |
| | pyridazinyl]phenol | |
| P-338 | 8-[3-(4-{4-[3-amino-6-(o- | m/z (ESI⁺) 527 (M + H)⁺. |
| | hydroxyphenyl)-4-pyridazinyl]-1,4- | |
| | diazepan-1-yl}-2-pyrimidinyl)-2- | |
| | propynyl]-8-azabicyclo[3.2.1]octan- | |
| | 3-ol | |
| P-339 | 6-(o-hydroxyphenyl)-4-(4-{2-[3- | ¹H NMR (499 MHz, d₆-DMSO) δ 14.22 (s, |
| | (2,2,2- | 1H), 8.30 (t, J = 5.8 Hz, 1H), 8.12 (s, 1H), 7.85 |
| | trifluoroethoxycarbonylamino)-1- | (s, 1H), 7.51 (s, 1H), 7.23 (t, J = 7.7 Hz, 1H), |
| | propynyl]-4-pyrimidinyl }-1,4- | 6.87 (d, J = 7.7 Hz, 2H), 6.74 (d, J = 6.3 Hz, |
| | diazepan-1-yl)-3-pyridazinamine | 1H), 6.18 (s, 2H), 4.70 (q, J = 9.1 Hz, 2H), |
| | | 4.09 (d, J = 5.8 Hz, 2H), 4.03 (s, 1H), 3.87 (s, |
| | | 2H), 3.76 (s, 1H), 3.66 (s, 1H), 3.45 - 3.40 (m, |
| | | 2H), 3.26 (t, J = 5.7 Hz, 2H), 2.04 (p, J = 5.8 |
| | | Hz, 2H). |
| P-340 | 0-[6-amino-5-(4-{2-[2-(tetrahydro-3- | ¹H NMR (499 MHz, d₆-DMSO) δ 14.20 (s, |
| | furyl)ethynyl]-4-pyrimidinyl }-1,4- | 1H), 7.84 (s, 1H), 7.51 (s, 1H), 7.26 - 7.19 (m, |
| | diazepan-1-yl)-3-pyridazinyl]phenol | 1H), 6.87 (d, J = 7.8 Hz, 2H), 6.75 (s, 1H), |
| | | 6.18 (s, 2H), 4.02 (s, 1H), 3.96 (t, J = 7.5 Hz, |
| | | 1H), 3.84 (q, J = 7.6 Hz, 1H), 3.74 (q, J = 7.4 |
| | | Hz, 1H), 3.61 (s, 2H), 3.43 (s, 2H), 3.26 (t, J = |
| | | 5.6 Hz, 3H), 2.27 (s, 1H), 2.04 (s, 2H), 1.95 (s, |
| | | 2H). |
| P-341 | 4-(4-{4-[3-amino-6-(0- | m/z. (ESI⁺) 446 (M + H)⁺. |
| | hydroxyphenyl)-4-pyridaziny1]-1,4- | |
| | diazepan-1-yl}-2-pyrimidinyl)-2- | |
| | methyl-3-butyn-2-ol | |
| P-342 | 0-(6-amino-5-{4-[2-(3-mcthoxy-1- | ¹H NMR (400 MHz, d₆-DMSO) δ 14.32 - |
| | propynyl)-4-pyrimidinyl]-1,4- | 14.13 (m, 1H), 8.31 - 8.05 (m, 1H), 8.01 - 7.75 |
| | diazepan-1-yl}-3-pyridazinyl)phenol | (m, 1H), 7.51 (s, 1H), 7.29 - 7.14 (m, 1H), 6.92 |
| | | - 6.82 (m, 2H), 6.75 (d, J = 6.4 Hz, 1H), 6.24 - |
| | | 6.10 (m, 2H), 4.33 (s, 2H), 4.19 - 3.58 (m, 4H), |
| | | 3.44 (s, 2H), 3.33 (s, 3H), 3.29 - 3.24 (m, 2H). |
| P-343 | N-[3-(4-{(2S,5S)-4-[3-amino-6-(0- | ¹H NMR (400 MHz, d₄-Methanol) δ 8.99 (d, J |
| | hydroxyphenyl)-4-pyridaziny1]-2,5- | = 6.8 Hz, 1H), 8.59 (dd, J = 1.6, 4.0 Hz, 1H), |
| | dimethyl-1-piperazinyl}-2- | 8.14 - 8.04 (m, 1H), 7.81 (s, 1H), 7.71 (d, J = |
| | pyrimidinyl)-2-propynyl]-1,4,7a- | 7.2 Hz, 1H), 7.28 - 7.21 (m, 1H), 7.16 - 7.10 |
| | triaza-2-indenecarboxamide | (m, 2H), 6.96 - 6.88 (m, 2H), 6.77 (d, J = 6.4 |
| | | Hz, 1H), 4.73 - 4.63 (m, 1H), 4.56 - 4.49 (m, |
| | | 1H), 4.48 (s, 2H), 3.53 - 3.46 (m, 1H), 3.20 |
| | | (dd, J = 2.0, 12.8 Hz, 1H), 3.15 - 3.04 (m, 1H), |
| | | 2.98 (dd, J = 3.2, 12.4 Hz, 1H), 1.46 (d, J = 6.4 |
| | | Hz, 3H), 1.12 (d, J = 6.0 Hz, 3H). |
| P-344 | 0-[6-amino-5-(8-{2-[3- | ¹H NMR (499 MHz, d₆-DMSO) δ 8.13 (s, 1H), |
| | (dimethylamino)-1-propynyl]-4- | 7.91 - 7.86 (m, 1H), 7.26 - 7.19 (m, 1H), 7.05 |
| | pyrimidinyl }-2,8-diaza-2- | (s, 1H), 6.86 (dd, J = 7.9, 5.8 Hz, 3H), 5.98 (s, |
| | spiro[4.5]decyl)-3-pyridazinyl]phenol | 2H), 3.74 (s, 2H), 3.67 (t, J = 7.0 Hz, 2H), 3.63 |
| | | (s, 2H), 3.47 (s, 2H), 3.43 (s, 2H), 2.26 (s, 6H), |
| | | 1.87 (t, J = 7.0 Hz, 2H), 1.69 - 1.55 (m, 4H). |
| P-345 | 0-{5-[(R)-4-{2-[3-(dimethylamino)-1- | ¹H NMR (400 MHz, d₄-Methanol) δ 8.27 (s, |
| | propynyl]-4-pyrimidinyl}-5-methyl- | 1H), 8.12 (d, J = 6.0 Hz, 1H), 7.73 (d, J = 7.6 |
| | 1,4-diazepan-1-y1]-6-amino-3- | Hz, 1H), 7.48 (s, 1H), 7.31 - 7.20 (m, 1H), 6.95 |
| | pyridazinyl }phenol | - 6.87 (m, 2H), 6.77 (d, J = 6.4 Hz, 1H), 4.18 - |
| | | 3.99 (m, 1H), 3.83 (s, 2H), 3.59 (s, 3H), 3.23 - |
| | | 3.00 (m, 2H), 2.90 - 2.77 (m, 1H), 2.64 (s, 6H), |
| | | 2.43 - 2.30 (m, 1H), 2.27 - 2.06 (m, 1H), 1.26 |
| | | (s, 3H). |
| P-346 | 0-[6-amino-5-(4-{2-[2-(1-methyl-4- | m/z (ESI⁺) 468 (M + H)⁺. |
| | pyrazolyl)ethynyl]-4-pyrimidinyl }- | |
| | 1,4-diazepan-1-yl)-3- | |
| | pyridazinyl]phenol | |
| P-347 | 0-[6-amino-5-(4-{2-[2-(3- | m/z (ESI⁺) 444 (M + H)⁺. |
| | oxetanyl)ethynyl]-4-pyrimidinyl }- | |
| | 1,4-diazepan-1-yl)-3- | |
| | pyridazinyl]phenol | |
| P-348 | tert-butyl 2-[2-(4-{4-[3-amino-6-(o- | m/z (ESI⁺) 557 (M + H)⁺. |
| | hydroxyphenyl)-4-pyridazinyl]-1,4- | |
| | diazepan-1-y1}-2- | |
| | pyrimidinyl)ethynyl]-1- | |
| | pyrrolidinecarboxylate | |
| P-349 | tert-butyl 4-[2-(4-{4-[3-amino-6-(o- | m/z (ESI⁺) 589 (M + H)⁺. |
| | hydroxyphenyl)-4-pyridazinyl]-1,4- | |
| | diazepan-1-yl}-2- | |
| | pyrimidinyl)ethynyl\|-4-fluoro-1- | |
| | piperidinecarboxylate | |
| P-350 | 0-[6-amino-5-(4-{2-[2-(1-methyl-2- | m/z (ESI⁺) 471 (M + H)⁺. |
| | pyrrolidinyl)cthynyl]-4-pyrimidinyl }- | |
| | 1,4-diazepan-1-yl)-3- | |
| | pyridazinyl]phenol | |
| P-351 | tert-butyl 4-[2-(4-{4-[3-amino-6-(o- | m/z (ESI⁺) 571 (M + H)⁺. |
| | hydroxyphenyl)-4-pyridazinyl]-1,4- | |
| | diazepan-1-yl}-2- | |
| | pyrimidinyl)ethynyl]-1- | |
| | piperidinecarboxylate | |
| P-352 | 0-[6-amino-5-(4-{2-[2-(1-mesyl-4- | m/z (ESI⁺) 549 (M + H)⁺. |
| | piperidyl)ethynyl]-4-pyrimidinyl }- | |
| | 1,4-diazepan-1-yl)-3- | |
| | pyridazinyl]phenol | |
| P-353 | 3-(4-{4-[3-amino-6-(o- | m/z (ESI⁺) 475 (M + H)⁺. |
| | hydroxyphenyl)-4-pyridaziny1]-1,4- | |
| | diazepan-1-y1}-2-pyrimidinyl)-2- | |
| | propynylaminomethylformylate | |
| P-354 | 0-[6-amino-5-(4-(2-[2-(1-methyl-2- | m/z (ESI⁺) 468 (M + H)⁺. |
| | imidazolyl)ethynyl]-4-pyrimidinyl}- | |
| | 1,4-diazepan-1-yl)-3- | |
| | pyridazinyl \|phenol | |
| P-355 | 0-[6-amino-5-(4-{2-[2-(hexahydro- | m/z (ESI⁺) 497 (M + H)⁺. |
| | 1H-pyrrolizin-7a-yl)ethynyl]-4- | |
| | pyrimidinyl}-1,4-diazepan-1-yl)-3- | |
| | pyridazinyl]phenol | |
| P-356 | 0-{5-[4-(2-{3-[(2R,6R)-2,6-dimethyl- | m/z (ESI⁺) 515 (M + H)⁺. |
| | 4-morpholinyl]-1-propynyl }-4- | |
| | pyrimidinyl)-1,4-diazepan-1-yl]-6- | |
| | amino-3-pyridazinyl}phenol | |
| P-357 | 1-[3-(4-{4-[3-amino-6-(o- | m/z (ESI⁺) 510 (M + H)⁺. |
| | hydroxyphenyl)-4-pyridazinyl]-1,4- | |
| | diazepan-1-yl}-2-pyrimidinyl)-2- | |
| | propynyl]-4-piperidinecarbonitrile | |
| P-358 | tert-butyl 3-[3-(4-{4-[3-amino-6-(o- | m/z (ESI⁺) 612 (M + H)⁺. |
| | hydroxyphenyl)-4-pyridazinyl]-1,4- | |
| | diazepan-1-y1}-2-pyrimidinyl)-2- | |
| | propynyl]-3,8- | |
| | diazabicyclo[3.2.1]octane-8- | |
| | carboxylate | |
| P-359 | 0-(6-amino-5-{ 4-[2-(2- | ¹H NMR (400 MHz, d₄-Methanol) δ 8.08 - |
| | cyclopropylethynyl)-4-pyrimidinyl]- | 7.90 (m, 1H), 7.65 (d, J = 6.4 Hz, 1H), 7.48 (s, |
| | 1,4-diazepan-1-yl}-3- | 1H), 7.24 (s, 1H), 6.91 (d, J = 7.2 Hz, 2H), |
| | pyridazinyl)phenol | 6.63 (s, 1H), 4.32 - 3.95 (m, 2H), 3.89 - 3.70 |
| | | (m, 2H), 3.55 (s, 2H), 3.39 (s, 2H), 2.13 (s, |
| | | 2H), 1.50 (d, J = 4.4 Hz, 1H), 0.98 - 0.92 (m, |
| | | 2H), 0.86 (d, J = 3.2 Hz, 2H). |
| P-360 | 0-[6-amino-5-(4-{2-[3-(2-ethyl-3,4- | m/z (ESI⁺) 563 (M + H)⁺. |
| | dihydro-2H-1,4-benzoxazin-4-yl)-1- | |
| | propynyl]-4-pyrimidinyl}-1,4- | |
| | diazepan-1-yl)-3-pyridazinyl]phenol | |
| P-361 | 1-[3-(4-{4-[3-amino-6-(o- | m/z (ESI⁺) 501 (M + H)⁺. |
| | hydroxyphenyl)-4-pyridazinyl]-1,4- | |
| | diazepan-1-yl}-2-pyrimidinyl)-2- | |
| | propynyl]-3-piperidinol | |
| P-362 | 8-[3-(4-{4-[3-amino-6-(o- | m/z (ESI⁺) 583 (M + H)⁺. |
| | hydroxyphenyl)-4-pyridazinyl]-1,4- | |
| | diazepan-1-yl}-2-pyrimidinyl)-2- | |
| | propynyl]-3-methyl-1,3,8-triaza-2,4- | |
| | spiro [4.5]decanedione | |
| P-363 | 0-{6-amino-5-[4-(2-{3-[3- | m/z (ESI⁺) 592 (M + H)⁺. |
| | (trifluoromethyl)-4,5,6,7-tetrahydro- | |
| | 1,2,3a,6-tetraaza-6-indenyl]-1- | |
| | propynyl}-4-pyrimidinyl)-1,4- | |
| | diazepan-1-yl]-3-pyridaziny1 } phenol | |
| P-364 | 4-[3-(4-{ 4-[3-amino-6-(o- | m/z (ESI⁺) 514 (M + H)⁺. |
| | hydroxyphenyl)-4-pyridazinyl]-1,4- | |
| | diazepan-1-yl}-2-pyrimidinyl)-2- | |
| | propynyl]-2,6-piperazinedione | |
| P-365 | {4-[3-(4-{4-[3-amino-6-(o- | m/z (ESI⁺) 554 (M + H)⁺. |
| | hydroxyphenyl)-4-pyridaziny1]-1,4- | |
| | diazepan-1-yl}-2-pyrimidinyl)-2- | |
| | propynyl]-1- | |
| | piperazinyl }cyclopropylmethanone | |
| P-366 | 0-[6-amino-5-(4-{2-[3-(1- | m/z (ESI⁺) 471 (M + H)⁺. |
| | pyrrolidinyl)-1-propynyl]-4- | |
| | pyrimidinyl }-1,4-diazepan-1-yl)-3- | |
| | pyridazinyl]phenol | |
| P-367 | N-[3-(4-{4-[3-amino-6-(5-fluoro-2- | m/z (ESI⁺) 580 (M + H)⁺. |
| | hydroxyphenyl)-4-pyridazinyl]-1,4- | |
| | diazepan-1-y1}-2-pyrimidinyl)-2- | |
| | propynyl]-1,4,7a-triaza-2- | |
| | indenecarboxamide | |
| P-368 | tert-butyl-1-[3-(4-{3-[6-(o- | m/z (ESI⁺) 597 (M + H)⁺. |
| | hydroxyphenyl)-4-pyridazinyl]-3,8- | |
| | diazabicyclo[3.2.1]oct-8-y1}-2- | |
| | pyrimidinyl)-2-propynyl]-4- | |
| | piperidylaminoformylate | |
| P-369 | 0-(5-{8-[2-(3-piperidino-1-propynyl)- | m/z (ESI⁺) 482 (M + H)⁺. |
| | 4-pyrimidinyl]-3,8- | |
| | diazabicyclo[3.2.1]oct-3-y1}-3- | |
| | pyridazinyl)phenol | |
| P-370 | 0-{6-amino-5-[4-(2-{3-(6-oxa-9-aza- | m/z (ESI⁺) 541 (M + H)⁺. |
| | 9-spiro[4.5]decyl)-1-propynyl}-4- | |
| | pyrimidinyl)-1,4-diazepan-1-yl]-3- | |
| | pyridazinyl }phenol | |
| P-371 | tert-butyl 3-[2-(4-{4-[3-amino-6-(o- | m/z (ESI⁺) 543 (M + H)⁺. |
| | hydroxyphenyl)-4-pyridaziny1]-1,4- | |
| | diazepan-1-yl}-2- | |
| | pyrimidinyl)ethynyl]-1- | |
| | azetidinecarboxylate | |
| P-372 | 1-[3-(4-{4-[3-amino-6-(o- | m/z (ESI⁺) 485 (M + H)⁺. |
| | hydroxyphenyl)-4-pyridaziny1]-1,4- | |
| | diazepan-1-y1}-2-pyrimidinyl)-2- | |
| | propynyl]-2-pyrrolidinone | |
| P-373 | 0-(6-amino-5-{4-[2-(3-piperidino-1- | m/z (ESI⁺) 485 (M + H)⁺. |
| | propynyl)-4-pyrimidinyl]-1,4- | |
| | diazepan-1-yl}-3-pyridazinyl)phenol | |
| P-374 | 3-[3-(4-{4-[3-amino-6-(o- | m/z (ESI⁺) 487 (M + H)⁺. |
| | hydroxyphenyl)-4-pyridazinyl]-1,4- | |
| | diazepan-1-yl }-2-pyrimidinyl)-2- | |
| | propynyl]-1,3-oxazolidin-2-one | |
| P-375 | 2-[3-(4-{4-[3-amino-6-(o- | m/z (ESI⁺) 547 (M + H)⁺. |
| | hydroxyphenyl)-4-pyridaziny1]-1,4- | |
| | diazepan-1-yl}-2-pyrimidinyl)-2- | |
| | propynyl]-1,3-isoindolinedione | |
| P-376 | 0-(5-{4-[2-(3-{(1R,4R)-2-oxa-5- | m/z (ESI⁺) 499 (M + H)⁺. |
| | azabicyclo[2.2.1]hept-5-y1}-1- | |
| | propynyl)-4-pyrimidinyl]-1,4- | |
| | diazepan-1-y1}-6-amino-3- | |
| | pyridazinyl)phenol | |
| P-377 | 0-(5-{4-[2-(3-{(1S,4S)-2-oxa-5- | m/z (ESI⁺) 499.1 (M + H)⁺. |
| | azabicyclo[2.2.1]hept-5-yl}-1- | |
| | propynyl)-4-pyrimidinyl]-1,4- | |
| | diazepan-1-y1}-6-amino-3- | |
| | pyridazinyl)phenol | |
| P-378 | 0-[6-amino-5-(4-{2-[3-(4-methoxy-1- | m/z (ESI⁺) 515 (M + H)⁺. |
| | piperidyl)-1-propynyl]-4- | |
| | pyrimidinyl}-1,4-diazepan-1-yl)-3- | |
| | pyridazinyl]phenol | |
| P-379 | 0-[6-amino-5-(4-{2-[3-(1,4-oxazepan- | m/z (ESI⁺) 501 (M + H)⁺. |
| | 4-yl)-1-propynyl]-4-pyrimidinyl }-1,4- | |
| | diazepan-1-yl)-3-pyridazinyl]phenol | |
| P-380 | 0-{5-[4-(2-{3-[(2R,6S)-2-methyl-6- | m/z (ESI⁺) 577 (M + H)⁺. |
| | phenyl-4-morpholinyl]-1-propynyl}- | |
| | 4-pyrimidinyl)-1,4-diazepan-1-yl]-6- | |
| | amino-3-pyridazinyl}phenol | |
| P-381 | 0-[5-(8-{2-[3-(dimethylamino)-1- | m/z (ESI⁺) 442 (M + H)⁺. |
| | propynyl]-4-pyrimidinyl}-3,8- | |
| | diazabicyclo[3.2.1]oct-3-yl)-3- | |
| | pyridazinyl]phenol | |
| P-382 | 0-[6-amino-5-(9-{2-[3- | m/z (ESI)⁺ 470.1 (M + H)⁺. |
| | (dimethylamino)-1-propynyl]-4- | |
| | pyridyl }-3,9-diazabicyclo[4.2.1]non- | |
| | 3-y1)-3-pyridazinyl]phenol | |
| P-383 | 0-(5-{(R)-4-[2-(3-isopropoxy-1- | m/z (ESI)⁺ 473.1 (M + H)⁺. |
| | propynyl)-4-pyridyl]-5-methyl-1,4- | |
| | diazepan-1-yl}-6-amino-3- | |
| | pyridazinyl)phenol | |
| P-384 | 0-[6-amino-5-(4-{2-[3- | m/z (ESI)⁺ 445.1 (M + H)⁺. |
| | (dimethylamino)-1-propynyl]-4- | |
| | pyridyloxy }-1-piperidyl)-3- | |
| | pyridazinyl]phenol | |
| P-385 | 6-{4-[3-amino-6-(o-hydroxyphenyl)- | m/z (ESI)⁺ 469.2 (M + H)⁺. |
| | 4-pyridazinyl]-1,4-diazepan-1-y1}-2- | |
| | [3-(dimethylamino)-1- | |
| | propynyl]nicotinonitrile | |
| P-386 | 0-[6-amino-5-(3-{2-[3- | ¹H NMR (400 MHz, d₆-DMSO) δ 14.97 - |
| | (dimethylamino)-1-propynyl]-4- | 13.52 (m, 1H), 8.40 (s, 1H), 8.26 - 8.04 (m, |
| | pyridyloxy}-1-azetidinyl)-3- | 1H), 7.92 (d, J = 7.6 Hz, 1H), 7.22 (t, J = 7.6 |
| | pyridazinyl]phenol | Hz, 1H), 7.07 - 7.00 (m, 2H), 6.98 - 6.93 (m, |
| | | 1H), 6.88 - 6.82 (m, 2H), 5.97 (s, 2H), 5.30 - |
| | | 5.22 (m, 1H), 4.65 (dd, J = 6.4, 9.6 Hz, 2H), |
| | | 4.21 (dd, J = 3.2, 9.6 Hz, 2H), 3.49 (s, 2H), |
| | | 2.26 (s, 6H). |
| P-387 | 0-(5-{(R)-5-methyl-4-[2-(3-methyl-1- | m/z (ESI)⁺ 443.3 (M + H)⁺. |
| | butynyl)-4-pyridyl]-1,4-diazepan-1- | |
| | yl}-6-amino-3-pyridazinyl)phenol | |
| P-388 | 0-(5-{(R)-4-[2-(2-cyclobutylethynyl)- | m/z (ESI)⁺ 455.3 (M + H)⁺. |
| | 4-pyridyl]-5-methyl-1,4-diazepan-1- | |
| | yl}-6-amino-3-pyridazinyl)phenol | |
| P-389 | 0-{5-[(1R,5S)-7-{2-[3- | m/z (ESI)⁺ 456.2 (M + H)⁺. |
| | (dimethylamino)-1-propynyl]-4- | |
| | pyridyl}-3,7-diazabicyclo[3.3.0]oct- | |
| | 3-yl]-6-amino-3-pyridazinyl} phenol | |
| P-390 | 1-[3-(4-{ (R)-4-[3-amino-6-(o- | m/z (ESI)⁺ 498.3 (M + H)⁺. |
| | hydroxyphenyl)-4-pyridaziny1]-7- | |
| | methyl-1,4-diazepan-1-yl}-2- | |
| | pyridyl)-2-propynyl]-2-pyrrolidinone | |
| P-391 | 0-(5-{(R)-4-[2-(2- | m/z (ESI)⁺ 441.3 (M + H)⁺. |
| | cyclopropylethynyl)-4-pyridyl]-5- | |
| | methyl-1,4-diazepan-1-yl}-6-amino- | |
| | 3-pyridazinyl)phenol | |
| P-392 | 0-{5-[(R)-4-(2-{3-(2- | m/z (ESI)⁺ 496.2 (M + H)⁺. |
| | azabicyclo[2.1.1]hex-2-yl)-1- | |
| | propynyl}-4-pyridyl)-5-methyl-1,4- | |
| | diazepan-1-yl]-6-amino-3- | |
| | pyridazinyl}phenol | |
| P-393 | 6-{(R)-4-[3-amino-6-(o- | m/z (ESI)⁺ 521.2 (M + H)⁺. |
| | hydroxyphenyl)-4-pyridazinyl]-7- | |
| | methyl-1,4-diazepan-1-yl}-2-{3-(2- | |
| | azabicyclo[2.1.1]hex-2-yl)-1- | |
| | propynyl } nicotinonitrile | |
| P-394 | 0-{5-[(R)-5-methyl-4-(6-[3-(1- | m/z (ESI)⁺ 552 (M + H)⁺. |
| | pyrrolidinyl)-1-propynyl]-5- | |
| | (trifluoromethyl)-2-pyridyl}-1,4- | |
| | diazepan-1-yl\|-6-amino-3- | |
| | pyridazinyl}phenol | |
| P-395 | 0-{5-[(R)-4-{6-[3-(dimethylamino)-1- | m/z (ESI)⁺ 526 (M + H)⁺. |
| | propynyl]-5-(trifluoromcthyl)-2- | |
| | pyridyl }-5-methyl-1,4-diazepan-1- | |
| | yl]-6-amino-3-pyridazinyl }phenol | |
| P-396 | 0-{5-[(R)-4-{2-[2-(3,3- | ¹H NMR (499 MHz, d₆-DMSO) δ 14.22 (s, |
| | difluorocyclobutyl)ethynyl]-4- | 1H), 8.05 (s, 1H), 7.88 (dd, J = 8.0, 1.6 Hz, |
| | pyridyl }-5-methyl-1,4-diazepan-1- | 1H), 7.55 (s, 1H), 7.22 (ddd, J = 8.4, 7.1, 1.6 |
| | yl]-6-amino-3-pyridazinyl }phenol | Hz, 1H), 6.90 - 6.83 (m, 2H), 6.80 (s, 1H), |
| | | 6.68 (s, 1H), 6.30 (s, 2H), 3.98 - 3.92 (m, 1H), |
| | | 3.71 (s, 2H), 3.52 - 3.40 (m, 2H), 3.24 (d, J = |
| | | 8.5 Hz, 1H), 3.03 (q, J = 10.6 Hz, 1H), 2.96 - |
| | | 2.87 (m, 1H), 2.78 - 2.69 (m, 1H), 2.24 (dt, J = |
| | | 14.4, 6.8 Hz, 1H), 2.09 (dt, J = 15.3, 10.3 Hz, |
| | | 1H), 1.13 (d, J = 6.1 Hz, 3H). |
| P-397 | 0-(5-{(R)-4-[2-(3-hydroxy-1- | m/z (ESI)⁺ 431.2 (M + H)⁺. |
| | propynyl)-4-pyridyl]-5-methyl-1,4- | |
| | diazepan-1-yl}-6-amino-3- | |
| | pyridazinyl)phenol | |
| P-398 | 0-(5-{(R)-4-[2-(3-methoxy-1- | m/z (ESI)⁺ 445.3 (M + H)⁺. |
| | propynyl)-4-pyridyl]-5-methyl-1,4- | |
| | diazepan-1-yl}-6-amino-3- | |
| | pyridazinyl)phenol | |
| P-399 | 0-{5-[(R)-4-{2-[3-(dimcthylamino)-1- | m/z (ESI)⁺ 472.3 (M + H)⁺. |
| | butynyl]-4-pyridyl }-5-methyl-1,4- | |
| | diazepan-1-y1]-6-amino-3- | |
| | pyridazinyl }phenol | |
| P-400 | 6-{(R)-4-[3-amino-6-(o- | m/z (ESI)⁺ 470.3 (M + H)⁺. |
| | hydroxyphenyl)-4-pyridazinyl]-7- | |
| | methyl-1,4-diazepan-1-yl}-2-(3- | |
| | methoxy-1-propynyl)nicotinonitrile | |
| P-401 | 0-{5-[(R)-4-{2-[(R)-3-hydroxy-1- | m/z (ESI)⁺ 445.2 (M + H)⁺. |
| | butynyl]-4-pyridyl }-5-methyl-1,4- | |
| | diazepan-1-yl]-6-amino-3- | |
| | pyridazinyl }phenol | |
| P-402 | 2-{3-[(S)-3-fluoro-1-pyrrolidinyl]-1- | m/z (ESI)⁺ 510.2 (M + H)⁺. |
| | propynyl }-6-{3-[6-(o- | |
| | hydroxyphenyl)-4-pyridazinyl]-3,8- | |
| | diazabicyclo[3.2.1]oct-8- | |
| | yl }nicotinonitrile | |
| P-403 | 2-[3-(3,3-difluoro-1-azetidinyl)-1- | m/z (ESI)⁺ 514.1 (M + H)⁺. |
| | propynyl]-6-{3-[6-(o- | |
| | hydroxyphenyl)-4-pyridazinyl]-3,8- | |
| | diazabicyclo[3.2.1]oct-8- | |
| | yl } nicotinonitrile | |
| P-404 | 0-{5-[(R)-4-[2-(3-{(1S,4S)-2-oxa-5- | ¹H NMR (499 MHz, d₆-DMSO) δ 8.07 (s, 2H), |
| | azabicyclo[2.2.1]hept-5-yl}-1- | 7.88 (d, J = 7.9 Hz, 1H), 7.55 (s, 1H), 7.22 (t, J |
| | propynyl)-4-pyridyl]-5-methyl-1,4- | = 7.6 Hz, 1H), 6.86 (dd, J = 10.5, 7.7 Hz, 2H), |
| | diazepan-1-yl]-6-amino-3- | 6.75 (s, 1H), 6.31 (s, 1H), 4.37 (s, 1H), 3.94 (s, |
| | pyridazinyl}phenol | 1H), 3.83 (d, J = 7.3 Hz, 1H), 3.70 (s, 1H), |
| | | 3.65 - 3.60 (m, 1H), 3.54 (s, 2H), 3.52 - 3.42 |
| | | (m, 2H), 2.95 (d, J = 8.4 Hz, 1H), 2.82 (s, 1H), |
| | | 2.73 (t, J = 11.8 Hz, 1H), 2.58 (s, 1H), 2.27 - |
| | | 2.19 (m, 1H), 2.10 (t, J = 13.4 Hz, 1H), 1.84 (d, |
| | | J = 8.9 Hz, 1H), 1.58 (d, J = 9.8 Hz, 1H), 1.23 |
| | | (s, 1H), 1.18 (d, J = 6.5 Hz, 6H), 1.14 (dd, J = |
| | | 16.0, 6.5 Hz, 3H). |
| P-405 | 4-{(R)-4-[3-amino-6-(0- | m/z (ESI)⁺ 507 (M + H)⁺. |
| | hydroxyphenyl)-4-pyridaziny1]-7- | |
| | methyl-1,4-diazepan-1-yl}-2-[3-(1H- | |
| | 1,2,4-triazol-1-yl)-1- | |
| | propynyl]nicotinonitrile | |
| P-406 | 4-{(R)-4-[3-amino-6-(0- | ¹H NMR (499 MHz, d₆-DMSO) δ 14.18 (s, |
| | hydroxyphenyl)-4-pyridazinyl]-7- | 1H), 8.16 (d, J = 6.5 Hz, 1H), 7.85 (dd, J = 8.0, |
| | methyl-1,4-diazepan-1-yl}-2-[3- | 1.6 Hz, 1H), 7.56 (s, 1H), 7.23 (ddd, J = 8.4, |
| | (dimethylamino)-1- | 7.1, 1.6 Hz, 1H), 6.93 (d, J = 6.6 Hz, 1H), 6.91 |
| | propynyl]nicotinonitrile | - 6.84 (m, 2H), 6.40 (s, 2H), 4.40 (dd, J = 11.0, |
| | | 5.8 Hz, 1H), 4.14 (dd, J = 16.2, 4.3 Hz, 1H), |
| | | 3.89 (dd, J = 16.3, 10.1 Hz, 1H), 3.63 - 3.53 |
| | | (m, 1H), 3.54 (s, 2H), 3.47 - 3.39 (m, 1H), |
| | | 2.97 (ddd, J = 20.1, 13.4, 9.8 Hz, 2H), 2.35 - |
| | | 2.27 (m, 1H), 2.27 (s, 7H), 2.07 (dt, J = 15.5, |
| | | 10.1 Hz, 1H), 1.25 (d, J = 6.2 Hz, 3H). |
| P-407 | 6-{(R)-4-[3-amino-6-(o- | m/z (ESI)⁺ 497.3 (M + H)⁺. |
| | hydroxyphenyl)-4-pyridazinyl]-7- | |
| | methyl-1,4-diazepan-1-yl}-2-[3- | |
| | (dimethylamino)-1- | |
| | butynyl]nicotinonitrile | |
| P-408 | 0-{5-[(R)-4-{5-chloro-2-[3-(1- | m/z (ESI)⁺ 516 (M + H)⁺. |
| | imidazolyl)-1-propynyl]-4-pyridyl}- | |
| | 5-methyl-1,4-diazepan-1-yl]-6-amino- | |
| | 3-pyridazinyl } phenol | |
| P-409 | 0-{5-[(R)-4-{5-chloro-2-[3- | ¹H NMR (499 MHz, d₆-DMSO) δ 14.12 (s, |
| | (dimethylamino)-1-propynyl]-4- | 1H), 8.27 (s, 1H), 7.89 (dd, J = 8.0, 1.6 Hz, |
| | pyridyl}-5-methyl-1,4-diazepan-1- | 1H), 7.52 (s, 1H), 7.23 (ddd, J = 8.5, 7.1, 1.6 |
| | yl]-6-amino-3-pyridazinyl }phenol | Hz, 1H), 7.19 (s, 1H), 6.91 - 6.83 (m, 2H), |
| | | 6.25 (s, 2H), 4.25 (q, J = 6.6 Hz, 1H), 3.92 (dd, |
| | | J = 15.6, 5.1 Hz, 1H), 3.70 (dd, J = 15.6, 9.1 |
| | | Hz, 1H), 3.50 (q, J = 8.7 Hz, 4H), 3.20 (dt, J = |
| | | 14.9, 8.2 Hz, 2H), 2.28 (s, 7H), 2.00 (dt, J = |
| | | 16.6, 8.9 Hz, 1H), 1.12 (d, J = 6.2 Hz, 3H). |
| P-410 | N-[3-(6-{(R)-4-[6-(0- | m/z (ESI)⁺ 585 (M + H)⁺. |
| | hydroxyphenyl)-4-pyridazinyl]-7- | |
| | methyl-1,4-diazepan-1-yl}-3-cyano- | |
| | 2-pyridyl)-2-propynyl]-1,4,7a-triaza- | |
| | 2-indenecarboxamide | |
| P-411 | 6-{(S)-4-[3-amino-6-(o- | m/z (ESI)⁺ 527 (M + H)⁺. |
| | hydroxyphenyl)-4-pyridazinyl]-7- | |
| | methyl-1,4-diazepan-1-yl}-2-{3-[(S)- | |
| | 3-fluoro-1-pyrrolidinyl]-1- | |
| | propynyl } nicotinonitrile | |
| P-412 | 6-{(R)-4-[3-amino-6-(0- | m/z (ESI)⁺ 531 (M + H)⁺. |
| | hydroxyphenyl)-4-pyridazinyl]-7- | |
| | methyl-1,4-diazepan-1-yl}-2-[3-(3,3- | |
| | difluoro-1-azetidinyl)-1- | |
| | propynyl]nicotinonitrile | |
| P-413 | 6-{(R)-4-[3-amino-6-(o- | m/z (ESI)⁺ 545 (M + H)⁺. |
| | hydroxyphenyl)-4-pyridazinyl]-7- | |
| | methyl-1,4-diazepan-1-y1}-2-{3-[(R)- | |
| | 3,3-difluoro-2-methyl-1-azetidinyl]-1- | |
| | propynyl } nicotinonitrile | |
| P-414 | 0-(5-{(R)-4-[5-fluoro-2-(3-piperidino- | ¹H NMR (499 MHz, d₆-DMSO) δ 8.13 (s, 2H), |
| | 1-propynyl)-4-pyridyl]-5-methyl-1,4- | 7.89 (dd, J = 8.0, 1.6 Hz, 1H), 7.55 (s, 1H), |
| | diazepan-1-yl}-6-amino-3- | 7.26 - 7.19 (m, 1H), 6.90 - 6.83 (m, 2H), 6.31 |
| | pyridazinyl)phenol | (s, 2H), 4.09 (dt, J = 11.4, 6.4 Hz, 1H), 3.82 (d, |
| | | J = 15.9 Hz, 1H), 3.72 (dd, J = 16.0, 10.0 Hz, |
| | | 1H), 3.55 (d, J = 12.5 Hz, 1H), 3.48 (s, 3H), |
| | | 3.49 - 3.42 (m, 1H), 3.05 (t, J = 11.5 Hz, 1H), |
| | | 2.91 (dd, J = 13.9, 9.5 Hz, 1H), 2.25 (dt, J = |
| | | 14.7, 7.0 Hz, 1H), 2.02 (dd, J = 25.7, 9.9 Hz, |
| | | 1H), 2.02 (s, 1H), 1.53 (p, J = 5.6 Hz, 5H), |
| | | 1.38 (d, J = 6.8 Hz, 3H), 1.19 (d, J = 6.1 Hz, |
| | | 3H). |
| P-415 | 2-{3-[N-methyl(2,2,2- | m/z (ESI)⁺ 551 (M + H)⁺. |
| | trifluoroethyl)amino]-1-propynyl}-6- | |
| | {(S)-4-[3-amino-6-(o- | |
| | hydroxyphenyl)-4-pyridazinyl]-7- | |
| | methyl-1,4-diazepan-1- | |
| | yl }nicotinonitrile | |
| P-416 | 6-{(R)-4-[3-amino-6-(o- | m/z (ESI)⁺ 559 (M + H)⁺. |
| | hydroxyphenyl)-4-pyridazinyl]-7- | |
| | methyl-1,4-diazepan-1-yl}-2-[3-(4,4- | |
| | difluoro-1-piperidyl)-1- | |
| | propynyl]nicotinonitrile | |
| P-417 | 0-{5-[(R)-4-{5-fluoro-2-[3-(1- | m/z (ESI)⁺ 499 (M + H)⁺. |
| | imidazolyl)-1-propynyl]-4-pyridyl }- | |
| | 5-methyl-1,4-diazepan-1-yl]-6-amino- | |
| | 3-pyridazinyl } phenol | |
| P-418 | 0-{5-[(R)-4-{2-[3-(2,4-dimethyl-1- | m/z (ESI)⁺ 498 (M + H)⁺. |
| | azetidinyl)-1-propynyl]-4-pyridy1}-5- | |
| | methyl-1,4-diazepan-1-yl]-6-amino-3- | |
| | pyridazinyl }phenol | |
| P-419 | 0-{5-[(R)-4-{2-[3-(dimethylamino)-1- | m/z (ESI)⁺ 476 (M + H)⁺. |
| | propynyl]-5-fluoro-4-pyridy1}-5- | |
| | methyl-1,4-diazepan-1-yl]-6-amino-3- | |
| | pyridazinyl }phenol | |
| P-420 | 0-{5-[(R)-4-(2-{3-[(R)-2-methyl-1- | m/z (ESI)⁺ 484 (M + H)⁺. |
| | azetidinyl]-1-propynyl }-4-pyridyl)-5- | |
| | methyl-1,4-diazepan-1-yl]-6-amino-3- | |
| | pyridaziny1}phenol | |
| P-421 | 0-{5-[(R)-4-{2-[3-(3,3-difluoro-1- | ¹H NMR (499 MHz, d₆-DMSO) δ 8.07 (s, 1H), |
| | azetidinyl)-1-propynyl]-4-pyridyl}-5- | 7.95 - 7.85 (m, 1H), 7.55 (s, 1H), 7.28 - 7.19 |
| | methyl-1,4-diazepan-1-yl]-6-amino-3- | (m, 1H), 6.97 - 6.83 (m, 2H), 6.73 (s, 1H), |
| | pyridazinyl }phenol | 6.31 (s, 1H), 3.98 - 3.92 (m, 1H), 3.72 (s, |
| | | 10H), 3.72 - 3.60 (m, 2H), 3.60 - 3.43 (m, |
| | | 1H), 2.92 (s, 1H), 2.75 (dd, J = 13.9, 9.5 Hz, |
| | | 1H), 2.24 (dt, J = 14.4, 6.9 Hz, 1H), 2.15 - |
| | | 2.04 (m, 1H), 1.26 (s, 1H), 1.20 - 1.03 (m, |
| | | 3H). |
| P-422 | 0-{5-[(R)-4-(2-{3-[(3R,5S)-3,5- | ¹H NMR (499 MHz, d₆-DMSO) δ 8.05 (s, 1H), |
| | dimethyl-4-morpholiny1]-1- | 7.88 (dd, J = 8.1, 1.6 Hz, 1H), 7.55 (s, 1H), |
| | propynyl }-4-pyridyl)-5-methyl-1,4- | 7.26 - 7.19 (m, 1H), 6.89 - 6.83 (m, 1H), 6.78 |
| | diazepan-1-yl]-6-amino-3- | (s, 1H), 6.68 (d, J = 9.9 Hz, 1H), 6.31 (s, 2H), |
| | pyridazinyl }phenol | 3.96 (dt, J = 12.0, 6.2 Hz, 1H), 3.79 (s, 2H), |
| | | 3.77 - 3.53 (m, 5H), 3.56 - 3.45 (m, 1H), 3.09 |
| | | (t, J = 10.8 Hz, 3H), 2.93 (dd, J = 13.1, 8.8 Hz, |
| | | 1H), 2.79 - 2.66 (m, 3H), 2.25 (dt, J = 14.5, 7.0 |
| | | Hz, 1H), 2.08 (dt, J = 15.4, 10.6 Hz, 1H), 1.26 |
| | | (d, J = 6.9 Hz, 1H), 1.13 (t, J = 6.4 Hz, 3H), |
| | | 0.91 (dd, J = 6.3, 3.2 Hz, 8H). |
| P-423 | 6-{(R)-4-[3-amino-6-(0- | ¹H NMR (499 MHz, d₆-DMSO) δ 14.22 (s, |
| | hydroxyphenyl)-4-pyridaziny1]-7- | 1H), 7.86 (d, J = 9.3 Hz, 2H), 7.53 (s, 1H), |
| | methyl-1,4-diazepan-1-yl}-2-[3- | 7.22 (ddd, J = 8.5, 7.2, 1.6 Hz, 1H), 6.90 - 6.82 |
| | (dimethylamino)-1- | (m, 2H), 6.32 (s, 1H), 3.82 (s, 1H), 3.56 (s, |
| | propynyl]nicotinonitrile | 3H), 2.86 (s, 1H), 2.77 (dd, J = 13.9, 9.4 Hz, |
| | | 1H), 2.28 (s, 5H), 2.29 - 2.20 (m, 1H), 1.14 (d, |
| | | J = 6.1 Hz, 2H). |
| P-424 | 0-{5-[(R)-5-methyl-4-{2-[3-(1H- | m/z (ESI)⁺ 482 (M + H)⁺. |
| | 1,2,4-triazol-1-yl)-1-propynyl]-4- | |
| | pyridyl}-1,4-diazepan-1-yl]-6-amino- | |
| | 3-pyridaziny1 } phenol | |
| P-425 | 0-(5-{(R)-5-methyl-4-[2-(3- | ¹H NMR (499 MHz, d₆-DMSO) δ 7.88 (dd, J = |
| | piperidino-1-propynyl)-4-pyridyl]- | 8.1, 1.6 Hz, 1H), 7.55 (s, 1H), 7.28 - 7.17 (m, |
| | 1,4-diazepan-1-yl}-6-amino-3- | 1H), 6.93 - 6.81 (m, 2H), 6.31 (s, 2H), 3.95 (s, |
| | pyridazinyl)phenol | 1H), 3.70 (d, J = 10.5 Hz, 2H), 3.59 - 3.42 (m, |
| | | 6H), 3.00 - 2.87 (m, 1H), 2.83 - 2.67 (m, 1H), |
| | | 2.24 (dt, J = 14.6, 6.9 Hz, 1H), 2.09 (dt, J = |
| | | 15.1, 10.3 Hz, 1H), 1.53 (q, J = 5.7 Hz, 4H), |
| | | 1.38 (s, 3H), 1.13 (d, J = 6.1 Hz, 3H). |
| P-426 | 0-(5-{(R)-5-methyl-4-[2-(3- | ¹H NMR (499 MHz, d₆-DMSO) δ 14.16 (s, |
| | morpholino-1-propynyl)-4-pyridyl]- | 1H), 8.05 (s, 1H), 7.88 (dd, J = 8.1, 1.6 Hz, |
| | 1,4-diazepan-1-yl}-6-amino-3- | 1H), 7.56 (s, 1H), 7.26 - 7.19 (m, 1H), 6.86 |
| | pyridazinyl)phenol | (td, J = 8.2, 2.4 Hz, 2H), 6.80 (s, 1H), 6.69 (s, |
| | | 1H), 6.31 (s, 2H), 3.96 (dt, J = 12.1, 6.3 Hz, |
| | | 1H), 3.71 (d, J = 10.3 Hz, 2H), 3.61 (t, J = 4.6 |
| | | Hz, 4H), 3.49 (s, 2H), 3.47 (s, 1H), 3.46 - 3.41 |
| | | (m, 1H), 2.93 (dd, J = 13.1, 9.1 Hz, 1H), 2.74 |
| | | (dd, J = 13.8, 9.4 Hz, 1H), 2.53 (s, 1H), 2.24 |
| | | (dt, J = 14.3, 6.8 Hz, 1H), 2.09 (dt, J = 15.5, |
| | | 10.3 Hz, 1H), 1.13 (d, J = 6.2 Hz, 3H). |
| P-427 | 0-{5-[(R)-4-{2-[3-(dimethylamino)-1- | m/z (ESI)⁺ 458 (M + H)⁺. |
| | propynyl]-4-pyridyl}-5-methyl-1,4- | |
| | diazepan-1-yl]-6-amino-3- | |
| | pyridazinyl }phenol | |
| P-428 | 0-{5-[(R)-4-{2-[3-(1-imidazolyl)-1- | m/z (ESI)⁺ 481 (M + H)⁺. |
| | propynyl]-4-pyridyl}-5-methyl-1,4- | |
| | diazepan-1-yl]-6-amino-3- | |
| | pyridazinyl }phenol | |
| P-429 | 6-{(R)-4-[3-amino-6-(o- | m/z (ESI)⁺ 506 (M + H)⁺. |
| | hydroxyphenyl)-4-pyridazinyl]-7- | |
| | methyl-1,4-diazepan-1-y1}-2-[3-(1- | |
| | imidazolyl)-1- | |
| | propynyl]nicotinonitrile | |
| P-430 | 3-(6-{(R)-4-[3-amino-6-(o- | m/z (ESI)⁺ 555 (M + H)⁺. |
| | hydroxyphenyl)-4-pyridazinyl]-7- | |
| | methyl-1,4-diazepan-1-yl}-3-cyano- | |
| | 2-pyridyl)-2-propynyl 2-methyl-2- | |
| | propanecarbamate | |
| P-431 | 3-(4-{(R)-4-[3-amino-6-(o- | ¹H NMR (499 MHz, d₆-DMSO) δ 14.24 (s, |
| | hydroxyphenyl)-4-pyridazinyl]-7- | 1H), 8.05 (d, J = 5.8 Hz, 1H), 7.91 - 7.81 (m, |
| | methyl-1,4-diazepan-1-yl}-2- | 1H), 7.54 (d, J = 16.4 Hz, 1H), 7.38 (d, J = 5.9 |
| | pyridyl)-2-propynyl 2-methyl-2- | Hz, 1H), 7.22 (td, J = 7.6, 1.6 Hz, 1H), 6.90 - |
| | propanecarbamate | 6.82 (m, 2H), 6.76 (s, 1H), 6.71 - 6.65 (m, |
| | | 1H), 6.29 (d, J = 9.0 Hz, 2H), 3.95 (dt, J = |
| | | 17.3, 5.8 Hz, 3H), 3.71 (s, 1H), 3.47 (t, J = 10.1 |
| | | Hz, 1H), 3.04 - 2.90 (m, 1H), 2.73 (dd, J = |
| | | 14.1, 9.5 Hz, 1H), 2.24 (dt, J = 14.3, 6.8 Hz, |
| | | 1H), 2.09 (dt, J = 15.3, 10.2 Hz, 1H), 1.40 (s, |
| | | 9H), 1.13 (d, J = 6.2 Hz, 3H). |
| P-432 | 3-(6-{4-[3-amino-6-(o- | m/z (ESI)⁺ 516 (M + H)⁺. |
| | hydroxyphenyl)-4-pyridaziny1]-1,4- | |
| | diazepan-1-yl}-2-pyridyl)-2-propynyl | |
| | 2-methyl-2-propanecarbamate | |

### Biological Examples

### SMARCA2 and SMARCA4 Degradation Assay

Degradation of protein which is expressed from the SMARCA2 and SMARCA4 genes was monitored using engineered HiBiT-fusion HeLa cell lines from Promega. In brief, 384-well white opaque plates (Greiner) were seeded with SMARCA2-HiBiT or SMARCA4-HiBiT HeLa cells at 8,000 cells/well and incubated overnight at 37° C. to allow cell attachment. After overnight incubation, test compounds were added in a 10-point dilution series (typically 10 μM to 300 pM) using a TECAN D300e Digital Dispenser, and plates were subsequently incubated for 24 hours at 37° C. 24-hours post-treatment, protein levels were quantified with the addition of HiBiT lytic buffer, LgBiT protein, and HiBiT substrate, according to manufacturer's specifications. Plates were incubated on an orbital plate shaker for 10 minutes at room temperature. Resulting luminescent values were read out using a ClarioStar plate reader and used to construct dose-response curves and calculation of degradation DC₅₀s (GraphPad Prism).

Table 4 provides data from the assays. Activity of the tested compounds is provided in Table 4 below as follows: A=DC₅₀<0.050 μM; B=0.05 μM<DC₅₀<0.50 μM; C=<0.5 μM<DC₅₀<5.0 μM; D=DC₅₀>5.0 μM. For SMARCA2 and SMARCA4 degradation activity @1 μM, +++=>70%; ++=30-70%; +=<30%.

TABLE 4

| | | SMARCA2 % | | SMARCA4 % |
|---|---|---|---|---|
| Com- | SMARCA2 | Degrada- | SMARCA4 | Degrada- |
| pound | DC₅₀ | tion @ 1 | DC₅₀ | tion @ 1 |
| # | (μM) | μM | (μM) | μM |
| P-1 | B | ++ | B | ++ |
| P-2 | B | ++ | B | ++ |
| P-3 | B | ++ | B | ++ |
| P-4 | B | ++ | B | + |
| P-5 | B | ++ | B | + |
| P-6 | B | ++ | A | ++ |
| P-7 | B | ++ | A | ++ |
| P-8 | B | ++ | A | ++ |
| P-9 | B | ++ | A | ++ |
| P-10 | B | ++ | A | ++ |
| P-11 | A | ++ | A | ++ |
| P-12 | B | ++ | B | ++ |
| P-14 | B | +++ | B | ++ |
| P-15 | C | ++ | C | + |
| P-16 | C | + | C | + |
| P-17 | C | ++ | C | ++ |
| P-18 | C | + | B | ++ |
| P-19 | C | ++ | C | ++ |
| P-20 | B | ++ | B | ++ |
| P-21 | B | ++ | B | ++ |
| P-22 | C | ++ | C | ++ |
| P-23 | B | ++ | B | ++ |
| P-24 | A | +++ | A | +++ |
| P-25 | B | +++ | B | +++ |
| P-26 | B | +++ | B | +++ |
| P-27 | B | +++ | B | +++ |
| P-28 | B | ++ | A | ++ |
| P-29 | B | +++ | B | +++ |
| P-30 | C | ++ | C | ++ |
| P-31 | B | ++ | B | ++ |
| P-32 | B | ++ | B | ++ |
| P-33 | B | ++ | B | ++ |
| P-34 | B | ++ | B | ++ |
| P-35 | B | ++ | B | +++ |
| P-36 | B | +++ | B | +++ |
| P-37 | B | ++ | B | ++ |
| P-38 | C | + | C | + |
| P-39 | C | + | C | + |
| P-40 | C | + | C | + |
| P-41 | B | ++ | B | ++ |
| P-42 | B | ++ | B | ++ |
| P-43 | C | ++ | B | ++ |
| P-44 | C | ++ | C | ++ |
| P-45 | C | + | C | + |
| P-46 | B | +++ | B | +++ |
| P-47 | C | + | C | + |
| P-48 | B | ++ | B | ++ |
| P-49 | D | + | C | + |
| P-50 | A | ++ | A | ++ |
| P-51 | B | ++ | B | ++ |
| P-52 | B | ++ | B | ++ |
| P-53 | B | ++ | B | ++ |
| P-54 | B | ++ | B | ++ |
| P-55 | B | ++ | B | ++ |
| P-56 | B | ++ | B | ++ |
| P-57 | B | ++ | A | ++ |
| P-58 | B | ++ | B | ++ |
| P-59 | B | ++ | D | |
| P-60 | B | ++ | B | ++ |
| P-61 | B | ++ | B | ++ |
| P-62 | B | ++ | B | ++ |
| P-63 | B | ++ | B | ++ |
| P-64 | B | ++ | B | ++ |
| P-65 | B | ++ | B | ++ |
| P-66 | B | ++ | B | ++ |
| P-67 | B | + | B | + |
| P-68 | B | + | D | |
| P-69 | B | ++ | A | ++ |
| P-70 | B | ++ | B | ++ |
| P-71 | B | ++ | B | ++ |
| P-72 | A | ++ | A | ++ |
| P-73 | B | ++ | B | ++ |
| P-74 | B | ++ | B | ++ |
| P-75 | B | ++ | A | ++ |
| P-76 | B | +++ | B | +++ |
| P-77 | C | + | B | ++ |
| P-78 | B | +++ | B | ++ |
| P-79 | C | ++ | B | ++ |
| P-80 | C | + | D | + |
| P-81 | B | ++ | A | ++ |
| P-82 | C | + | C | + |
| P-83 | C | + | B | + |
| P-84 | C | + | D | + |
| P-85 | C | + | C | + |
| P-86 | C | + | D | |
| P-87 | C | + | | |
| P-88 | D | + | | |
| P-89 | C | + | C | + |
| P-90 | C | + | C | + |
| P-91 | C | + | C | + |
| P-92 | B | + | B | ++ |
| P-93 | B | ++ | A | ++ |
| P-94 | B | ++ | B | + |
| P-95 | B | + | A | ++ |
| P-96 | C | ++ | B | +++ |
| P-97 | B | ++ | B | ++ |
| P-98 | B | ++ | B | ++ |
| P-99 | C | + | C | + |
| P-100 | B | +++ | B | ++ |
| P-101 | A | +++ | A | +++ |
| P-102 | B | +++ | A | +++ |
| P-103 | B | +++ | B | ++ |
| P-104 | B | +++ | C | ++ |
| P-105 | B | +++ | A | +++ |
| P-106 | C | ++ | C | ++ |
| P-107 | A | +++ | A | +++ |
| P-108 | B | +++ | B | +++ |
| P-109 | A | +++ | B | ++ |
| P-110 | B | +++ | B | +++ |
| P-111 | B | +++ | B | ++ |
| P-112 | B | +++ | B | +++ |
| P-113 | B | ++ | C | + |
| P-114 | B | +++ | B | ++ |
| P-115 | B | +++ | B | ++ |
| P-116 | B | +++ | B | ++ |
| P-117 | B | ++ | B | ++ |
| P-118 | B | ++ | B | ++ |
| P-119 | D | + | C | + |
| P-120 | C | ++ | C | ++ |
| P-121 | B | +++ | B | +++ |
| P-122 | C | ++ | C | + |
| P-123 | B | ++ | A | ++ |
| P-124 | B | ++ | B | +++ |
| P-125 | B | ++ | B | ++ |
| P-126 | B | ++ | B | ++ |
| P-127 | B | ++ | B | +++ |
| P-128 | A | +++ | B | ++ |
| P-129 | B | ++ | B | ++ |
| P-130 | B | +++ | B | ++ |
| P-131 | B | ++ | B | ++ |
| P-132 | C | ++ | C | ++ |
| P-133 | B | ++ | B | ++ |
| P-134 | B | ++ | A | ++ |
| P-135 | B | ++ | B | ++ |
| P-136 | B | ++ | B | ++ |
| P-137 | C | ++ | C | ++ |
| P-138 | B | +++ | B | ++ |
| P-139 | B | +++ | C | +++ |
| P-140 | C | ++ | C | ++ |
| P-141 | C | ?? | C | ++ |
| P-142 | B | +++ | B | +++ |
| P-143 | B | +++ | B | ++ |
| P-144 | B | +++ | B | ++ |
| P-145 | B | +++ | B | ++ |
| P-146 | B | +++ | B | ++ |
| P-147 | B | ++ | B | ++ |
| P-148 | B | +++ | B | ++ |
| P-149 | B | | B | ++ |
| P-150 | B | | B | ++ |
| P-151 | B | ++ | B | ++ |
| P-152 | B | +++ | B | ++ |
| P-153 | B | ++ | B | ++ |
| P-154 | B | ++ | B | + |
| P-155 | B | ++ | B | ++ |
| P-156 | B | ++ | C | + |
| P-157 | A | ++ | B | ++ |
| P-158 | B | ?? | B | ++ |
| P-159 | B | +++ | B | ++ |
| P-160 | B | +++ | B | ++ |
| P-161 | B | ++ | B | ++ |
| P-162 | B | ++ | B | ++ |
| P-163 | B | ++ | B | + |
| P-164 | A | +++ | A | ++ |
| P-165 | B | +++ | B | ++ |
| P-166 | B | +++ | B | +++ |
| P-167 | A | ++ | A | + |
| P-168 | B | +++ | B | +++ |
| P-169 | B | +++ | B | +++ |
| P-170 | B | ++ | C | ++ |
| P-171 | B | ++ | C | + |
| P-172 | A | ++ | A | ++ |
| P-173 | A | +++ | B | ++ |
| P-174 | B | +++ | B | ++ |
| P-175 | B | ++ | B | + |
| P-176 | B | ++ | B | ++ |
| P-177 | B | ++ | B | + |
| P-178 | B | +++ | B | ++ |
| P-179 | B | +++ | B | ++ |
| P-180 | A | ++ | A | ++ |
| P-181 | B | ++ | A | ++ |
| P-182 | B | ++ | B | ++ |
| P-183 | B | +++ | B | ++ |
| P-184 | A | +++ | A | ++ |
| P-185 | B | +++ | B | +++ |
| P-186 | B | +++ | B | ++ |
| P-187 | B | ++ | B | ++ |
| P-188 | A | +++ | A | ++ |
| P-189 | A | ++ | A | ++ |
| P-190 | B | +++ | B | ++ |
| P-191 | B | ++ | B | ++ |
| P-192 | A | +++ | B | ++ |
| P-193 | B | ++ | B | + |
| P-194 | A | ++ | A | ++ |
| P-195 | B | ++ | B | ++ |
| P-196 | C | ++ | C | + |
| P-197 | B | ++ | C | ++ |
| P-198 | B | +++ | C | ++ |
| P-199 | B | +++ | B | ++ |
| P-200 | B | +++ | B | ++ |
| P-201 | B | +++ | B | ++ |
| P-202 | A | +++ | A | ++ |
| P-203 | B | ++ | B | ++ |
| P-204 | B | ++ | C | ++ |
| P-205 | D | | C | ++ |
| P-206 | A | +++ | B | +++ |
| P-207 | C | ++ | C | + |
| P-208 | B | ++ | B | + |
| P-209 | B | ++ | B | ++ |
| P-210 | A | ++ | A | ++ |
| P-211 | B | ++ | C | + |
| P-212 | B | ++ | B | ++ |
| P-213 | B | ++ | D | + |
| P-214 | B | ++ | B | ++ |
| P-215 | B | +++ | B | ++ |
| P-216 | A | ++ | A | ++ |
| P-217 | B | ++ | B | + |
| P-218 | B | +++ | B | ++ |
| P-219 | B | +++ | B | +++ |
| P-220 | B | +++ | B | ++ |
| P-221 | B | +++ | B | +++ |
| P-222 | B | +++ | B | +++ |
| P-223 | A | ++ | A | ++ |
| P-224 | B | +++ | B | +++ |
| P-225 | A | ++ | A | ++ |
| P-226 | A | ++ | A | + |
| P-227 | B | +++ | B | ++ |
| P-228 | A | ++ | A | ++ |
| P-229 | A | +++ | B | ++ |
| P-230 | B | ++ | B | + |
| P-231 | A | ++ | A | ++ |
| P-232 | A | +++ | A | ++ |
| P-233 | A | ++ | A | ++ |
| P-234 | A | +++ | A | ++ |
| P-235 | B | + | C | + |
| P-236 | A | ++ | A | + |
| P-237 | A | +++ | A | ++ |
| P-238 | B | +++ | B | ++ |
| P-239 | B | ++ | B | ++ |
| P-240 | C | ++ | C | + |
| P-241 | B | ++ | B | ++ |
| P-242 | A | ++ | A | ++ |
| P-243 | B | ++ | B | ++ |
| P-244 | A | ++ | A | ++ |
| P-245 | B | +++ | B | ++ |
| P-246 | A | ++ | A | ++ |
| P-247 | B | ++ | B | ++ |
| P-248 | C | ++ | C | + |
| P-249 | A | ++ | A | + |
| P-250 | A | +++ | A | +++ |
| P-251 | B | +++ | B | ++ |
| P-252 | B | ++ | B | ++ |
| P-253 | B | ++ | B | ++ |
| P-254 | A | ++ | A | ++ |
| P-255 | B | ++ | B | ++ |
| P-256 | B | ++ | C | ++ |
| P-257 | B | +++ | B | ++ |
| P-258 | C | ++ | C | ++ |
| P-259 | A | ++ | A | ++ |
| P-260 | A | ++ | B | + |
| P-261 | B | +++ | B | ++ |
| P-262 | B | +++ | B | ++ |
| P-263 | B | ++ | A | + |
| P-264 | A | ++ | A | ++ |
| P-265 | B | +++ | B | +++ |
| P-266 | B | +++ | B | ++ |
| P-267 | B | +++ | B | ++ |
| P-268 | B | +++ | B | ++ |
| P-269 | B | +++ | B | +++ |
| P-270 | B | ++ | B | ++ |
| P-271 | B | ++ | B | + |
| P-272 | B | ++ | B | ++ |
| P-273 | B | +++ | B | ++ |
| P-274 | B | +++ | B | ++ |
| P-275 | B | +++ | B | ++ |
| P-276 | B | ++ | B | ++ |
| P-277 | B | ++ | B | ++ |
| P-278 | B | ++ | B | ++ |
| P-279 | B | +++ | B | ++ |
| P-280 | A | ++ | A | + |
| P-281 | A | ++ | A | ++ |
| P-282 | B | ++ | B | ++ |
| P-283 | B | ++ | B | + |
| P-284 | B | ++ | C | + |
| P-285 | A | ++ | A | + |
| P-286 | B | ++ | B | ++ |
| P-287 | B | ++ | B | ++ |
| P-288 | B | ++ | B | ++ |
| P-289 | B | ++ | B | ++ |
| P-290 | B | ++ | B | + |
| P-291 | B | ++ | B | + |
| P-292 | B | ++ | A | + |
| P-293 | B | ++ | A | + |
| P-294 | A | ++ | A | + |
| P-295 | B | ++ | B | + |
| P-296 | B | ++ | B | ++ |
| P-297 | B | ++ | A | + |
| P-298 | B | +++ | B | ++ |
| P-299 | B | +++ | B | ++ |
| P-300 | B | +++ | B | ++ |
| P-301 | B | ++ | C | ++ |
| P-302 | B | ?? | B | ++ |
| P-303 | A | ++ | A | ++ |
| P-304 | B | +++ | B | |
| P-305 | B | ++ | B | ++ |
| P-306 | C | ?? | C | + |
| P-307 | B | ++ | B | ++ |
| P-308 | C | ++ | C | ++ |
| P-309 | B | ++ | B | ++ |
| P-310 | B | ++ | B | ++ |
| P-311 | B | ++ | C | ++ |
| P-312 | B | +++ | B | ++ |
| P-313 | B | ++ | C | + |
| P-314 | B | | B | ++ |
| P-315 | B | ++ | C | ++ |
| P-316 | B | ++ | C | + |
| P-317 | B | ++ | C | + |
| P-318 | B | ++ | B | ++ |
| P-319 | A | ++ | A | + |
| P-320 | B | ++ | A | ++ |
| P-321 | B | ++ | B | ++ |
| P-322 | B | ++ | B | ++ |
| P-323 | B | ++ | B | ++ |
| P-324 | C | ++ | C | + |
| P-325 | C | ++ | B | ++ |
| P-326 | C | ++ | C | + |
| P-327 | C | ++ | C | ++ |
| P-328 | B | ++ | B | ++ |
| P-329 | B | ++ | B | + |
| P-330 | B | ++ | B | + |
| P-331 | B | ++ | B | + |
| P-332 | A | ++ | B | + |
| P-333 | B | ++ | B | + |
| P-334 | B | ++ | B | + |
| P-335 | A | ++ | A | ++ |
| P-336 | B | ++ | B | + |
| P-337 | B | +++ | B | +++ |
| P-338 | B | ++ | B | + |
| P-339 | B | +++ | B | +++ |
| P-340 | B | ++ | B | ++ |
| P-341 | C | ++ | C | + |
| P-342 | B | ++ | B | ++ |
| P-343 | B | ++ | B | ++ |
| P-344 | A | ++ | D | |
| P-345 | A | ++ | A | ++ |
| P-346 | C | ++ | C | + |
| P-347 | B | +++ | B | +++ |
| P-348 | B | +++ | B | +++ |
| P-349 | B | ++ | B | ++ |
| P-350 | B | +++ | B | +++ |
| P-351 | C | ++ | C | ++ |
| P-352 | C | ++ | C | ++ |
| P-353 | B | +++ | B | +++ |
| P-354 | C | ++ | C | ++ |
| P-355 | B | +++ | B | +++ |
| P-356 | C | ++ | C | + |
| P-357 | B | ++ | C | ++ |
| P-358 | C | ++ | C | ++ |
| P-359 | B | ++ | B | + |
| P-360 | B | ++ | B | ++ |
| P-361 | C | ++ | C | + |
| P-362 | B | ++ | C | ++ |
| P-363 | C | ++ | C | ++ |
| P-364 | C | ++ | C | + |
| P-365 | C | ++ | C | ++ |
| P-366 | B | +++ | B | +++ |
| P-367 | C | ++ | C | + |
| P-368 | B | ++ | B | ++ |
| P-369 | A | ++ | C | + |
| P-370 | B | +++ | B | ++ |
| P-371 | B | +++ | B | ++ |
| P-372 | B | +++ | B | ++ |
| P-373 | B | +++ | B | ++ |
| P-374 | B | +++ | B | ++ |
| P-375 | B | +++ | B | ++ |
| P-376 | B | ++ | C | ++ |
| P-377 | B | ++ | C | ++ |
| P-378 | B | +++ | B | ++ |
| P-379 | B | +++ | B | ++ |
| P-380 | C | ++ | C | + |
| P-381 | B | ++ | D | |
| P-382 | A | +++ | A | +++ |
| P-383 | A | +++ | A | ++ |
| P-384 | B | ++ | B | + |
| P-385 | B | +++ | B | +++ |
| P-386 | A | + | A | |
| P-387 | C | ++ | C | + |
| P-388 | B | ++ | C | ++ |
| P-389 | B | ++ | A | ++ |
| P-390 | B | +++ | B | +++ |
| P-391 | B | ++ | B | |
| P-392 | B | +++ | B | +++ |
| P-393 | A | +++ | A | ++ |
| P-394 | C | + | C | |
| P-395 | C | | D | |
| P-396 | B | +++ | B | ++ |
| P-397 | B | ++ | C | ++ |
| P-398 | B | ++ | C | ++ |
| P-399 | A | ++ | A | ++ |
| P-400 | C | | D | |
| P-401 | B | +++ | B | +++ |
| P-402 | B | ++ | B | + |
| P-403 | C | | D | |
| P-404 | B | ++ | B | ++ |
| P-405 | C | | D | |
| P-406 | C | ++ | C | ++ |
| P-407 | A | ++ | A | + |
| P-408 | B | ++ | B | ++ |
| P-409 | B | + | B | |
| P-410 | D | | D | |
| P-411 | B | ++ | B | + |
| P-412 | B | ++ | B | + |
| P-413 | B | ++ | D | |
| P-414 | B | + | B | ++ |
| P-415 | B | ++ | B | ++ |
| P-416 | B | +++ | B | +++ |
| P-417 | B | ++ | B | ++ |
| P-418 | A | +++ | A | ++ |
| P-419 | B | + | B | + |
| P-420 | A | +++ | A | ++ |
| P-421 | B | ++ | A | ++ |
| P-422 | B | + | A | |
| P-423 | A | ++ | A | ++ |
| P-424 | B | +++ | B | ++ |
| P-425 | A | ++ | A | ++ |
| P-426 | B | ++ | B | ++ |
| P-427 | A | +++ | A | ++ |
| P-428 | B | +++ | B | +++ |
| P-429 | B | +++ | B | ++ |
| P-430 | C | ++ | C | + |
| P-431 | B | ++ | B | ++ |
| P-432 | C | | D | |

Unless otherwise defined, all technical and scientific terms used herein have the same meaning as commonly understood by one of ordinary skill in the art to which this disclosure belongs.

The disclosure illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claims.

All publications, patent applications, patents, and other references mentioned herein are expressly incorporated by reference in their entirety, to the same extent as if each were incorporated by reference individually. In case of conflict, the present specification, including definitions, will control. It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

What is claimed is:

1. A compound of formula I: 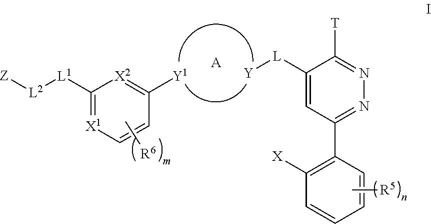
or a pharmaceutically acceptable salt, solvate, stereoisomer, or tautomer thereof, wherein:
X is hydroxy, halo, cyano, C₁₋₄ alkoxy, or —N(R)₂;
Y is N, C, or CH;
Y¹ is N, C, or CH;
X¹ is either CH, CR⁶, or N;
X² is either CH, CR⁶, or N; wherein at least one of X¹ or X² is N;
T is —N(R)₂;
Ring A is C₄₋₈ cycloalkyl, C₆₋₁₀ aryl, 4- to 11-membered heterocycloalkyl, or 5- to 10-membered heteroaryl, wherein each heterocycloalkyl or heteroaryl have from 1 to 3 heteroatoms selected from O, N, NR, and S, and wherein each cycloalkyl, aryl, heterocycloalkyl, or heteroaryl is independently unsubstituted or substituted with one to four R¹;
L is a bond, —CH₂— or —O—;
L¹ is —RC=CR— or —C≡C—;
L² is selected from: 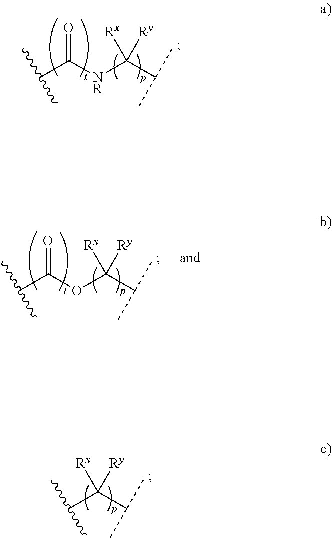
wherein t is 0 or 1, p is 0, 1, 2, or 3, and wherein the dashed line ( [symbol] ) denotes the point of attachment from L² to L¹, and the wavy line ( [symbol] ) denotes the point of attachment from L² to Z;
Z is C₁₋₁₀ alkyl, —N(R)₂, —OH, C₁₋₁₀ alkoxy, C₃₋₈ cycloalkyl, 4- to 10-membered heterocycloalkyl, or 5- to 10-membered heteroaryl, wherein each heterocycloalkyl or heteroaryl have from 1 to 4 heteroatoms selected from NR, N, O, and S, and wherein each heterocycloalkyl or heteroaryl is independently unsubstituted or substituted with one to four R², and wherein each alkyl, alkoxy, or cycloalkyl is independently unsubstituted or substituted with one to three R³;
each R is independently hydrogen, C₁₋₄ alkyl, or C₃₋₆ cycloalkyl, wherein each alkyl or cycloalkyl is unsubstituted or substituted with one to three R⁴; or
two R groups, when attached to the same nitrogen, form a 4- to 7-membered heterocycloalkyl, which is unsubstituted or substituted with one to four R⁴;
each R^{x} and R^{y} is independently hydrogen, C₁₋₄ alkyl, or C₁₋₄ haloalkyl substituted with 1 to 3 halo; or
R^{x} and R^{y}, together with the atoms to which they are attached, form a C₃₋₆ cycloalkyl, which is unsubstituted or substituted with one to four R⁴;
each R¹ is independently cyano, halo, hydroxyl, nitro, oxo, —N(R)₂, C₁₋₄ alkyl, C₁₋₄ haloalkyl substituted with 1 to 3 halo, C₁₋₄ alkoxy unsubstituted or substituted with 1 to 3 halo, C₅₋₆ heteroaryl having from 1 to 3 heteroatoms selected from O, N, NR, and/or S, 4- to 7-membered heterocycloalkyl having from 1 to 3 heteroatoms selected from oxygen, nitrogen, and/or sulfur, —C(O)OR, —OC(O)R, or —C(O)R;
each R² is independently oxo, hydroxy, cyano, halo, —N(R)₂, —C(O)OR, —NRC(O)OR, C₁₋₄ alkyl, C₁₋₄ haloalkyl substituted with from 1 to 3 halo groups, C₁₋₄ alkoxy, C₁₋₄ alkoxy substituted with from 1 to 3 halo groups, C₁₋₄ alkyl substituted with C₁₋₂ alkoxy, C₅₋₆ heteroaryl having from 1 to 3 heteroatoms selected from O, N, and NR, or C₃₋₆ cycloalkyl;
each R³ is independently oxo, cyano, hydroxy, halogen, C₁₋₄ alkyl, C₁₋₄ alkoxy, C₁₋₄ alkoxy substituted with from 1 to 3 halo groups, C₁₋₄ haloalkyl substituted with from 1 to 3 halo groups, C₁₋₆ aryl, C₁₋₄ alkyl substituted with 1 to 3 phenyl, or C₃₋₆ cycloalkyl;
each R⁴ is independently —N(R)₂, cyano, halo, or hydroxyl;
each R⁵ is D, halo, C₁₋₃ haloalkyl, or C₁₋₄ alkoxy;
each R⁶ is N(R)₂, SR, halo, cyano, C₁₋₄ alkoxy unsubstituted or substituted with 1 to 3 halo, unsubstituted C₁₋₄ alkyl, C₁₋₄ alkyl substituted with C₁₋₂ alkoxy, or C₁₋₄ alkyl substituted with hydroxy;
m is 0, 1, or 2; and
n is 0, 1, 2, or 3;
with the proviso that when the compound 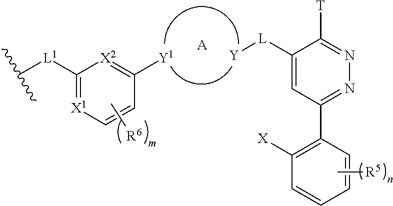
is the following moiety: 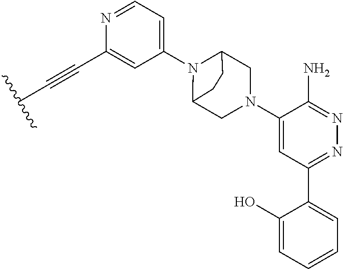 [SMILES: *C#Cc1cc(N2C3CCC2CN(c2cc(-c4ccccc4O)nnc2N)C3)ccn1]
and L² is a —C(O)NH—, bond, —CH₂—, or —(CH₂)₂—, then Z is not a heterocyclyl (wherein the heterocyclyl is unsubstituted or substituted), —C₁₋₆ alkylene-NH₂, or —C₁₋₆ alkylene-OH,
and the compound is not:
2-(6-amino-5-(8-(2-(3-((2-hydroxyethyl)(methyl)amino)prop-1-yn-1-yl)pyridin-4-yl)-3,8-diazabicyclo[3.2.1]octan-3-yl)pyridazin-3-yl)phenol,
2-(6-amino-5-(8-(2-(3-(2-hydroxyethoxyprop-1-yn-1-yl)pyridin-4-yl)-3,8-diazabicyclo[3.2.1]octan-3-yl)pyridazin-3-yl)phenol,
2-(6-amino-5-(8-(2-(3-(diethylamino)prop-1-yn-1-yl)pyridin-4-yl)-3,8-diazabicyclo[3.2.1]octan-3-yl)pyridazin-3-yl)phenol,
2-(6-amino-5-(8-(2-(3-(piperidin-3-yloxy)prop-1-yn-1-yl)pyridin-4-yl)-3,8-diazabicyclo[3.2.1]octan-3-yl)pyridazin-3-yl)phenol,
2-(6-amino-5-(8-(2-((1-methyl-1H-pyrazol-4-yl)ethynyl)pyridin-4-yl)-3,8-diazabicyclo[3.2.1]octan-3-yl)pyridazin-3-yl)phenol,
N-[3-[4-[3-[3-amino-6-(2-hydroxyphenyl)pyridazin-4-yl]-3,8-diazabicyclo[3.2.1]octan-8-yl]-2-pyridyl]prop-2-ynyl]piperidine-4-carboxamide,
2-[6-amino-5-[8-[2-[3-(azepan-1-yl)prop-1-ynyl]pyrimidin-4-yl]-3,8-diazabicyclo[3.2.1]octan-3-yl]pyridazin-3-yl]phenol,
2-[6-amino-5-[9-[2-[3-(azepan-yl)prop-1-ynyl]-4-pyridyl]-3,9-diazaspiro[5.5]undecan-3-yl]pyridazin-3-yl]phenol,
2-[6-amino-5-[2-[2-[3-(azepan-1-yl)prop-1-ynyl]-4-pyridyl]-2,8-diazaspiro[4.5]decan-8-yl]pyridazin-3-yl]phenol,
2-[6-amino-5-[6-[2-[3-(azepan-1-yl)prop-1-ynyl]-4-pyridyl]-2,6-diazaspiro[3.3]heptan-2-yl]pyridazin-3-yl]phenol,
2-(6-amino-5-(8-(2-(((1r,4r)-4-aminocyclohexyl)ethynyl)pyridin-4-yl)-3,8-diazabicyclo[3.2.1]octan-3-yl)pyridazin-3-yl)phenol, or
2-[6-amino-5-[8-[2-[(E)-3-(azepan-1-yl)propl-enyl]-4-pyridyl]-3,8-diazabicyclo[3.2.1]octan-3-yl]pyridazin-3-yl]phenol.

2. A compound of formula IA: 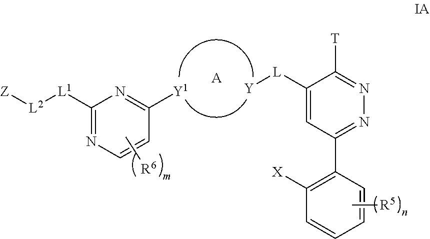
or a pharmaceutically acceptable salt, solvate, stereoisomer, or tautomer thereof, wherein:
X is hydroxy, halo, cyano, C₁₋₄ alkoxy, or —N(R)₂;
Y is N, C, or CH;
Y¹ is N, C, or CH;
T is —N(R)₂;
Ring A is C₄₋₈ cycloalkyl, C₆₋₁₀ aryl, 4- to 11-membered heterocycloalkyl, or 5- to 10-membered heteroaryl, wherein each heterocycloalkyl or heteroaryl have from 1 to 3 heteroatoms selected from O, N, NR, and S, and wherein each cycloalkyl, aryl, heterocycloalkyl, or heteroaryl is independently unsubstituted or substituted with one to four R¹;
L is a bond, —CH₂—, or —O—;
L¹ is —RC=CR— or —C≡C—;
L² is selected from: 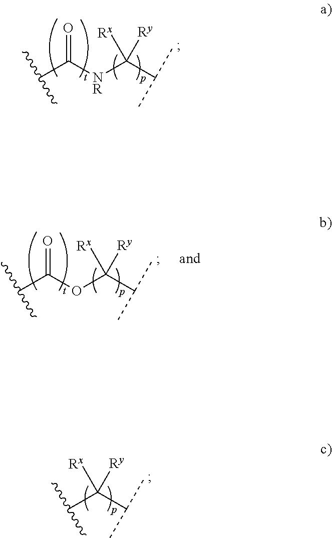
wherein t is 0 or 1, p is 0, 1, 2, or 3, and wherein the dashed line ( [symbol] ), denotes the point of attachment from L² to L¹, and the wavy line ( [symbol] ) denotes the point of attachment from L² to Z;
Z is C₁₋₁₀ alkyl, —N(R)₂, —OH, C₁₋₁₀ alkoxy, C₃₋₈ cycloalkyl, 4- to 10-membered heterocycloalkyl, or 5- to 10-membered heteroaryl, wherein each heterocycloalkyl or heteroaryl have from 1 to 4 heteroatoms selected from NR, N, O, and S, and wherein each heterocycloalkyl or heteroaryl is independently unsubstituted or substituted with one to four R², and wherein each alkyl, alkoxy, or cycloalkyl is independently unsubstituted or substituted with one to three R³;
each R is independently hydrogen, C₁₋₄ alkyl, or C₃₋₆ cycloalkyl, wherein each alkyl or cycloalkyl is unsubstituted or substituted with one to three R⁴; or
two R groups, when attached to the same nitrogen, form a 4- to 7-membered heterocycloalkyl, which is unsubstituted or substituted with one to four R⁴;
each R^{x} and R^{y} is independently hydrogen, C₁₋₄ alkyl, or C₁₋₄ haloalkyl substituted with 1 to 3 halo; or
R^{x} and R^{y}, together with the atoms to which they are attached, form a C₃₋₆ cycloalkyl, which is unsubstituted or substituted with one to four R⁴;
each R¹ is independently cyano, halo, hydroxyl, nitro, oxo, —N(R)₂, C₁₋₄ alkyl, C₁₋₄ haloalkyl substituted with 1 to 3 halo, C₁₋₄ alkoxy unsubstituted or substituted with 1 to 3 halo, C₅₋₆ heteroaryl having from 1 to 3 heteroatoms selected from O, N, NR, and/or S, 4- to 7-membered heterocycloalkyl having from 1 to 3 heteroatoms selected from oxygen, nitrogen, and/or sulfur, —C(O)OR, —OC(O)R, or —C(O)R;
each R² is independently oxo, hydroxy, cyano, halo, —N(R)₂, —C(O)OR, —NRC(O)OR, C₁₋₄ alkyl, C₁₋₄ haloalkyl substituted with from 1 to 3 halo groups, C₁₋₄ alkoxy, C₁₋₄ alkoxy substituted with from 1 to 3 halo groups, C₁₋₄ alkyl substituted with C₁₋₂ alkoxy, C₅₋₆ heteroaryl having from 1 to 3 heteroatoms selected from O, N, and NR, or C₃₋₆ cycloalkyl;
each R³ is independently oxo, cyano, hydroxy, halogen, C₁₋₄ alkyl, C₁₋₄ alkoxy, C₁₋₄ alkoxy substituted with from 1 to 3 halo groups, C₁₋₄ haloalkyl substituted with from 1 to 3 halo groups, C₁₋₆ aryl, C₁₋₄ alkyl substituted with 1 to 3 phenyl, or C₃₋₆ cycloalkyl;
each R⁴ is independently —N(R)₂, cyano, halo, or hydroxyl;
each R⁵ is D, halo, C₁₋₃ haloalkyl, or C₁₋₄ alkoxy;
each R⁶ is N(R)₂, SR, halo, cyano, C₁₋₄ alkoxy unsubstituted or substituted with 1 to 3 halo, unsubstituted C₁₋₄ alkyl, C₁₋₄ alkyl substituted with C₁₋₂ alkoxy, or C₁₋₄ alkyl substituted with hydroxy;
m is 0, 1, or 2; and
n is 0, 1, 2, or 3;
with the proviso that the compound is not 2-[6-amino-5-[8-[2-[3-(azepan-1-yl)prop-1-ynyl]pyrimidin-4-yl]-3,8-diazabicyclo[3.2.1]octan-3-yl]pyridazin-3-yl]phenol.

3. A compound of formula I′: 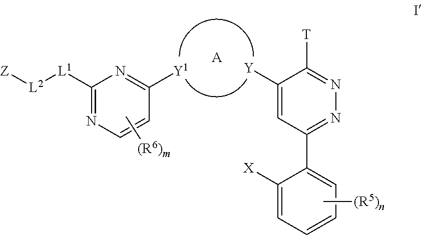
or a pharmaceutically acceptable salt, solvate, stereoisomer, or tautomer thereof, wherein:
X is hydroxy, halo, cyano, C₁₋₄ alkoxy, or —N(R)₂;
Y is N, C, or CH;
Y¹ is N, C, or CH;
T is —N(R)₂;
Ring A is C₄₋₈ cycloalkyl, C₆₋₁₀ aryl, 4- to 11-membered heterocycloalkyl, or 5- to 10-membered heteroaryl, wherein each heterocycloalkyl or heteroaryl have from 1 to 3 heteroatoms selected from O, N, NR, and S, and wherein each cycloalkyl, aryl, heterocycloalkyl, or heteroaryl is independently unsubstituted or substituted with one to four R¹;
L¹ is —RC=CR— or —C≡C—;
L² is selected from: 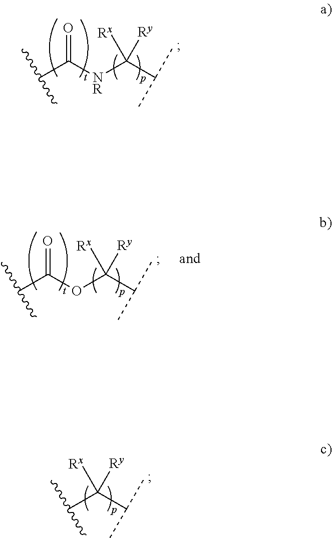
wherein t is 0 or 1, p is 0, 1, 2, or 3, and wherein the dashed line ( [symbol] ) denotes the point of attachment from L² to L¹, and the wavy line ( [symbol] ) denotes the point of attachment from L² to Z;
Z is C₁₋₁₀ alkyl, —N(R)₂, —OH, C₁₋₁₀ alkoxy, C₃₋₈ cycloalkyl, 4- to 10-membered heterocycloalkyl, or 5- to 10-membered heteroaryl, wherein each heterocycloalkyl or heteroaryl have from 1 to 4 heteroatoms selected from NR, N, O, and S, and wherein each heterocycloalkyl or heteroaryl is independently unsubstituted or substituted with one to four R², and wherein each alkyl, alkoxy, or cycloalkyl is independently unsubstituted or substituted with one to three R³;
each R is independently hydrogen, C₁₋₄ alkyl, or C₃₋₆ cycloalkyl, wherein each alkyl or cycloalkyl is unsubstituted or substituted with one to three R⁴; or
two R groups, when attached to the same nitrogen, form a 4- to 7-membered heterocycloalkyl, which is unsubstituted or substituted with one to four R⁴;
each R^{x} and R^{y} is independently hydrogen, C₁₋₄ alkyl, or C₁₋₄ haloalkyl substituted with 1 to 3 halo; or
R^{x} and R^{y}, together with the atoms to which they are attached, form a C₃₋₆ cycloalkyl, which is unsubstituted or substituted with one to four R⁴;
each R¹ is independently cyano, halo, hydroxyl, nitro, oxo, —N(R)₂, C₁₋₄ alkyl, C₁₋₄ haloalkyl substituted with 1 to 3 halo, C₁₋₄ alkoxy unsubstituted or substituted with 1 to 3 halo, C₅₋₆ heteroaryl having from 1 to 3 heteroatoms selected from O, N, NR, and/or S, 4- to 7-membered heterocycloalkyl having from 1 to 3 heteroatoms selected from oxygen, nitrogen, and/or sulfur, —C(O)OR, —OC(O)R, or —C(O)R;
each R² is independently oxo, hydroxy, cyano, halo, —N(R)₂, —C(O)OR, —NRC(O)OR, C₁₋₄ alkyl, C₁₋₄ haloalkyl substituted with from 1 to 3 halo groups, C₁₋₄ alkoxy, C₁₋₄ alkoxy substituted with from 1 to 3 halo groups, C₁₋₄ alkyl substituted with C₁₋₂ alkoxy, C₅₋₆ heteroaryl having from 1 to 3 heteroatoms selected from O, N, and NR, or C₃₋₆ cycloalkyl;
each R³ is independently oxo, halogen, C₁₋₄ alkyl, C₁₋₄ alkoxy, C₁₋₄ alkoxy substituted with from 1 to 3 halo groups, C₁₋₄ haloalkyl substituted with from 1 to 3 halo groups, or C₃₋₆ cycloalkyl;
each R⁴ is independently —N(R)₂, cyano, halo, or hydroxyl;
each R⁵ is D, halo, C₁₋₃ haloalkyl, or C₁₋₄ alkoxy;
each R⁶ is N(R)₂, SR, cyano, C₁₋₄ alkoxy unsubstituted or substituted with 1 to 3 halo, C₁₋₄ alkyl substituted with C₁₋₂ alkoxy, or C₁₋₄ alkyl substituted with hydroxy;
m is 0, 1, or 2; and
n is 0, 1, 2, or 3;
with the proviso that the compound is not 2-[6-amino-5-[8-[2-[3-(azepan-1-yl)prop-1-ynyl]pyrimidin-4-yl]-3,8-diazabicyclo[3.2.1]octan-3-yl]pyridazin-3-yl]phenol.

4. The compound of claim 3, wherein the compound of Formula I′ is represented by Formula II′: 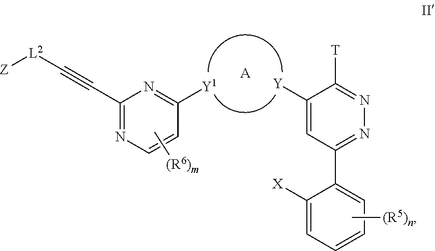
or a pharmaceutically acceptable salt, solvate, stereoisomer, or tautomer thereof.

5. The compound of claim 4, wherein the compound of Formula II′ is represented by Formula IIa′: 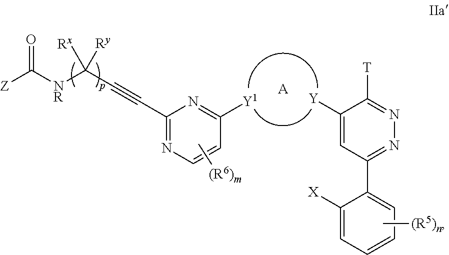
or a pharmaceutically acceptable salt, solvate, stereoisomer, or tautomer thereof.

6. The compound of claim 5, wherein the compound of Formula IIa′ is represented by Formula IIa′-1: 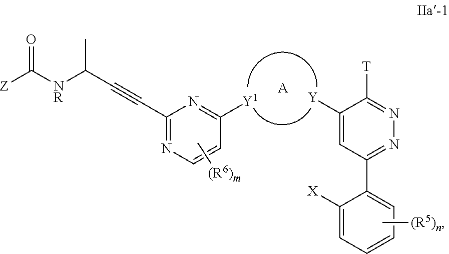
or a pharmaceutically acceptable salt, solvate, stereoisomer, or tautomer thereof.

7. The compound of claim 5, wherein the compound of Formula IIa′ is represented by Formula IIa′-2: 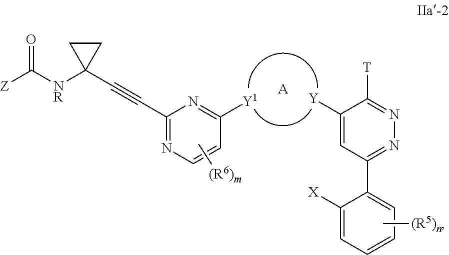
or a pharmaceutically acceptable salt, solvate, stereoisomer, or tautomer thereof.

8. The compound of claim 4, wherein the compound of Formula II′ is represented by Formula IIb′: 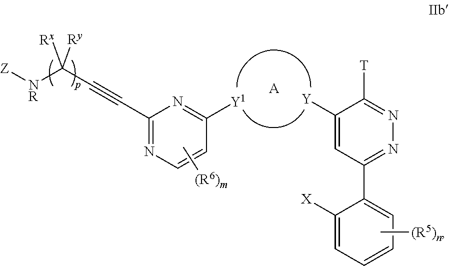
or a pharmaceutically acceptable salt, solvate, stereoisomer, or tautomer thereof.

9. The compound of claim 4, wherein the compound of Formula II′ is represented by Formula IIc′: 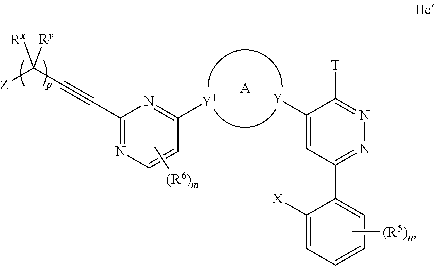
or a pharmaceutically acceptable salt, solvate, stereoisomer, or tautomer thereof.

10. The compound of claim 4, wherein the compound of Formula II′ is represented by Formula IId′: 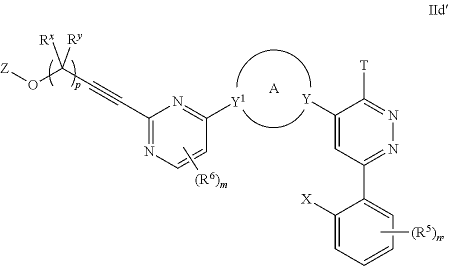
or a pharmaceutically acceptable salt, solvate, stereoisomer, or tautomer thereof.

11. The compound of claim 3, wherein the compound of Formula I′ is represented by Formula III′-E: 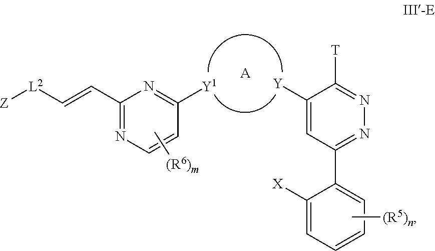
or a pharmaceutically acceptable salt, solvate, stereoisomer, or tautomer thereof.

12. The compound of claim 11, wherein the compound of Formula III′-E is represented by Formula IIIa′-E: 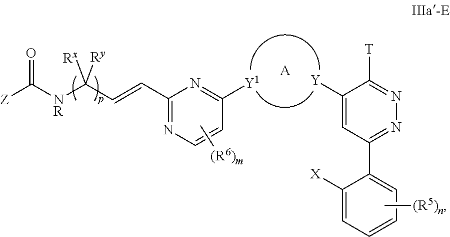
or a pharmaceutically acceptable salt, solvate, stereoisomer, or tautomer thereof.

13. The compound of claim 11, wherein the compound of Formula III′-E is represented by Formula IIIb′-E: 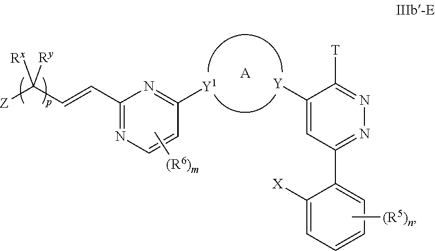
or a pharmaceutically acceptable salt, solvate, stereoisomer, or tautomer thereof.

14. The compound of claim 3, wherein the compound of Formula I′ is represented by Formula III′-Z: 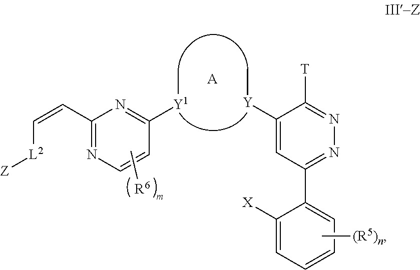
or a pharmaceutically acceptable salt, solvate, stereoisomer, or tautomer thereof.

15. The compound of claim 14, wherein the compound of Formula III′-Z is represented by Formula IIIa′-Z: 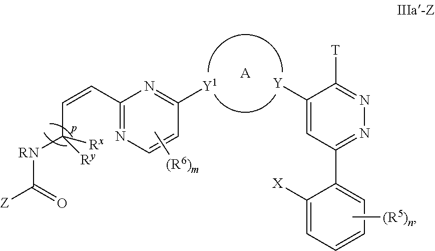
or a pharmaceutically acceptable salt, solvate, stereoisomer, or tautomer thereof.

16. The compound of claim 14, wherein the compound of Formula III′-Z is represented by Formula IIIb′-Z: 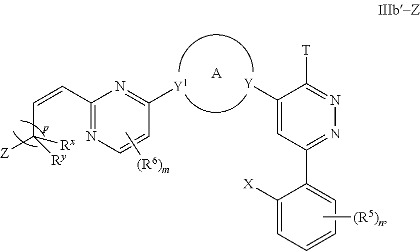
or a pharmaceutically acceptable salt, solvate, stereoisomer, or tautomer thereof.

17. The compound of any one of claims 1-16, or a pharmaceutically acceptable salt, solvate, stereoisomer, or tautomer thereof, wherein 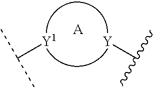
is selected from 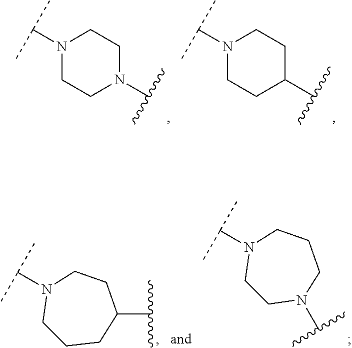
wherein the dashed line ( [symbol] ) denotes the point of attachment from Ring A to pyrimidine, and the wavy line ( [symbol] ) denotes the point of attachment from Ring A to L or pyridazine, and wherein each Ring A is independently unsubstituted or substituted with one to four R¹, wherein each R¹ is independently halo or C₁₋₄ alkyl.

18. The compound of any one of claims 1-16, or a pharmaceutically acceptable salt, solvate, stereoisomer, or tautomer thereof, wherein 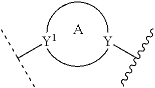
is selected from 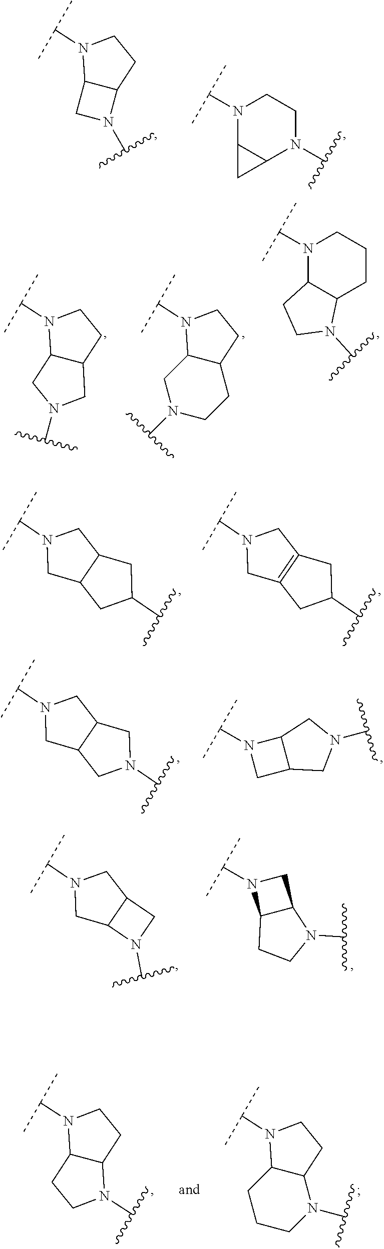
wherein the dashed line ( [symbol] ) denotes the point of attachment from Ring A to pyrimidine, and the wavy line ( [symbol] ) denotes the point of attachment from Ring A to L or pyridazine, and wherein each Ring A is independently unsubstituted or substituted with one to four RI, wherein each R¹ is independently halo or C₁₋₄ alkyl.

19. The compound of any one of claims 1-16, or a pharmaceutically acceptable salt, solvate, stereoisomer, or tautomer thereof, wherein 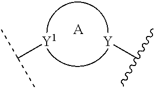
is selected from 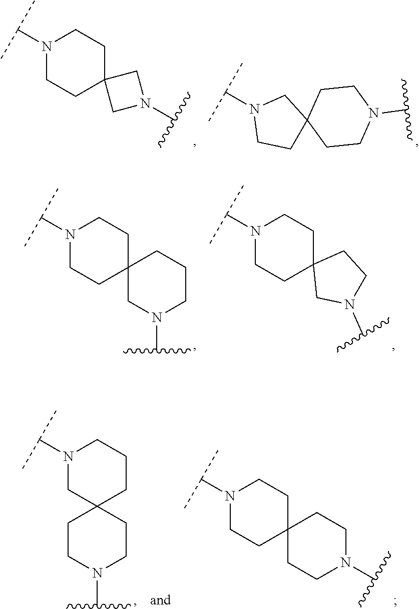
wherein the dashed line ( [symbol] ) denotes the point of attachment from Ring A to pyrimidine, and the wavy line ( [symbol] ) denotes the point of attachment from Ring A to L or pyridazine, and wherein each Ring A is independently unsubstituted or substituted with one to four R¹, wherein each R¹ is independently halo or C₁₋₄ alkyl.

20. The compound of any one of claims 1-16, or a pharmaceutically acceptable salt, solvate, stereoisomer, or tautomer thereof, wherein 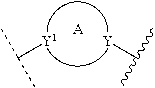
is selected from 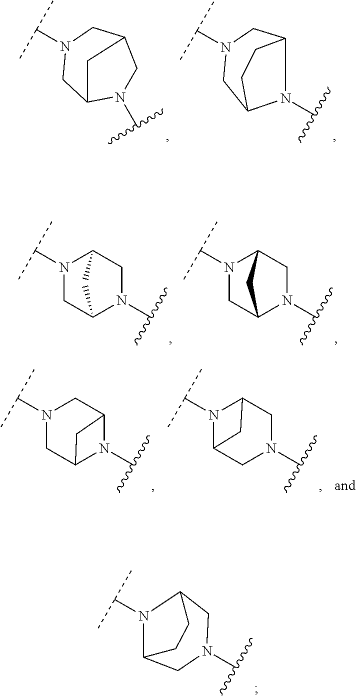
wherein the dashed line ( [symbol] ) denotes the point of attachment from Ring A to pyrimidine, and the wavy line ( [symbol] ) denotes the point of attachment from Ring A to L or pyridazine, and wherein each Ring A is independently unsubstituted or substituted with one to four R¹, wherein each R¹ is independently halo or C₁₋₄ alkyl.

21. The compound of any one of claims 1-20, or a pharmaceutically acceptable salt, solvate, stereoisomer, or tautomer thereof, wherein Z is selected from 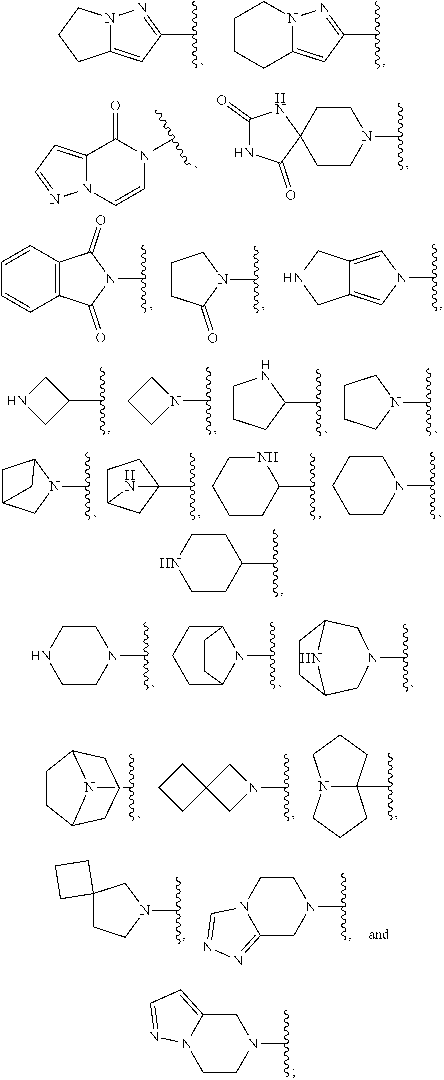
wherein each Z is independently unsubstituted or substituted with one to four R², wherein each R² is independently oxo, halo, —C(O)OR, —NRC(O)OR, C₁₋₄ alkyl, C₁₋₄ haloalkyl substituted with from 1 to 3 halo groups, C₅₋₆ heteroaryl having from 1 to 3 heteroatoms selected from O, N, and NR, or C₃₋₆ cycloalkyl.

22. The compound of any one of claims 1-20, or a pharmaceutically acceptable salt, solvate, stereoisomer, or tautomer thereof, wherein Z is selected from 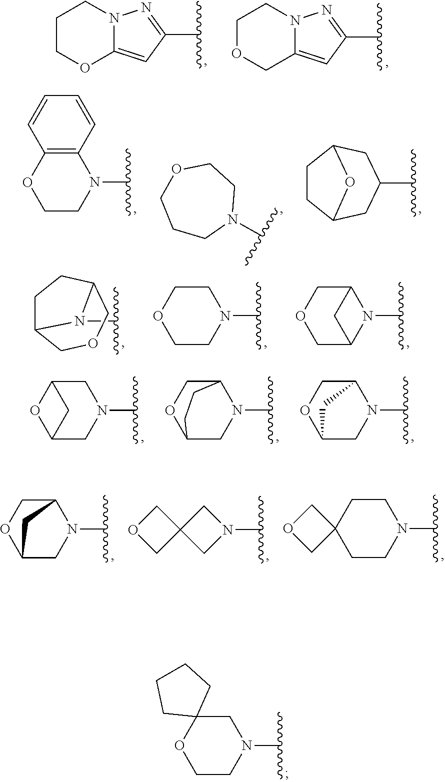
wherein each Z is independently unsubstituted or substituted with one to four R², wherein each R² is independently oxo, halo, —C(O)OR, —NRC(O)OR, C₁₋₄ alkyl, C₁₋₄ haloalkyl substituted with from 1 to 3 halo groups, C₅₋₆ heteroaryl having from 1 to 3 heteroatoms selected from O, N, and NR, or C₃₋₆ cycloalkyl.

23. The compound of any one of claims 1-20, or a pharmaceutically acceptable salt, solvate, stereoisomer, or tautomer thereof, wherein Z is 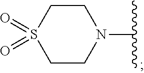
wherein Z is independently unsubstituted or substituted with one to four R², wherein each R² is independently oxo, halo, —C(O)OR, —NRC(O)OR, C₁₋₄ alkyl, C₁₋₄ haloalkyl substituted with from 1 to 3 halo groups, C₅₋₆ heteroaryl having from 1 to 3 heteroatoms selected from O, N, and NR, or C₃₋₆ cycloalkyl.

24. The compound of any one of claims 1-20, or a pharmaceutically acceptable salt, solvate, stereoisomer, or tautomer thereof, wherein Z is selected from 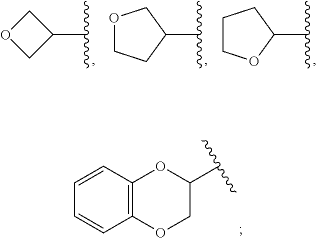
wherein Z is independently unsubstituted or substituted with one to four R², wherein each R² is independently oxo, halo, —C(O)OR, —NRC(O)OR, C₁₋₄ alkyl, C₁₋₄ haloalkyl substituted with from 1 to 3 halo groups, C₅₋₆ heteroaryl having from 1 to 3 heteroatoms selected from O, N, and NR, or C₃₋₆ cycloalkyl.

25. The compound of any one of claims 1-20, or a pharmaceutically acceptable salt, solvate, stereoisomer, or tautomer thereof, wherein Z is selected from 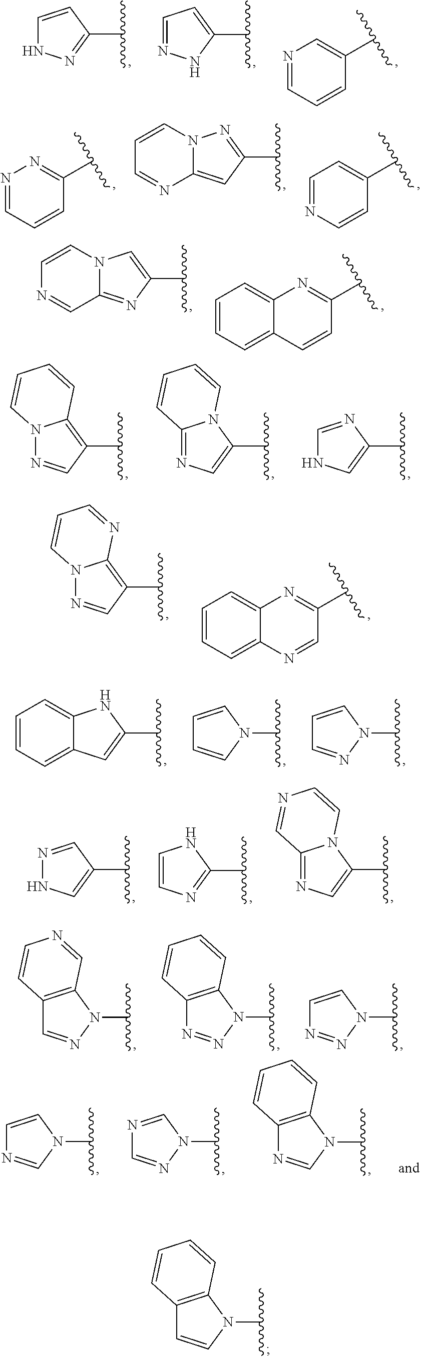
wherein each Z is independently unsubstituted or substituted with one to four R², wherein each R² is independently oxo, halo, —C(O)OR, —NRC(O)OR, C₁₋₄ alkyl, C₁₋₄ haloalkyl substituted with from 1 to 3 halo groups, C₅₋₆ heteroaryl having from 1 to 3 heteroatoms selected from O, N, and NR, or C₃₋₆ cycloalkyl.

26. The compound of any one of claims 1-20, or a pharmaceutically acceptable salt, solvate, stereoisomer, or tautomer thereof, wherein Z is selected from 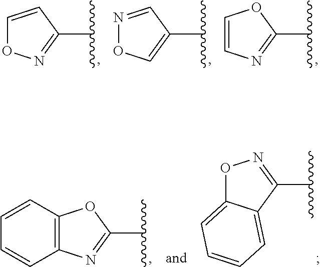
wherein each Z is independently unsubstituted or substituted with one to four R², wherein each R² is independently oxo, halo, —C(O)OR, —NRC(O)OR, C₁₋₄ alkyl, C₁₋₄ haloalkyl substituted with from 1 to 3 halo groups, C₅₋₆ heteroaryl having from 1 to 3 heteroatoms selected from O, N, and NR, or C₃₋₆ cycloalkyl.

27. The compound of any one of claims 1-20, or a pharmaceutically acceptable salt, solvate, stereoisomer, or tautomer thereof, wherein Z is selected from 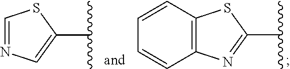
wherein each Z is independently unsubstituted or substituted with one to four R², wherein each R² is independently oxo, halo, —C(O)OR, —NRC(O)OR, C₁₋₄ alkyl, C₁₋₄ haloalkyl substituted with from 1 to 3 halo groups, C₅₋₆ heteroaryl having from 1 to 3 heteroatoms selected from O, N, and NR, or C₃₋₆ cycloalkyl.

28. The compound of any one of claims 1-20, or a pharmaceutically acceptable salt, solvate, stereoisomer, or tautomer thereof, wherein Z is selected from 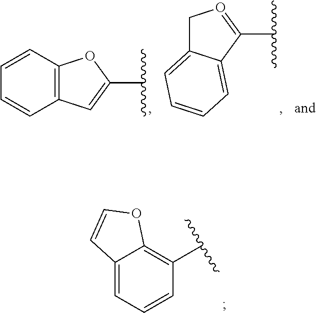
wherein each Z is independently unsubstituted or substituted with one to four R², wherein each R² is independently oxo, halo, —C(O)OR, —NRC(O)OR, C₁₋₄ alkyl, C₁₋₄ haloalkyl substituted with from 1 to 3 halo groups, C₅₋₆ heteroaryl having from 1 to 3 heteroatoms selected from O, N, and NR, or C₃₋₆ cycloalkyl.

29. The compound of any one of claims 1-20, or a pharmaceutically acceptable salt, solvate, stereoisomer, or tautomer thereof, wherein Z is C₁₋₁₀ alkyl.

30. The compound of any one of claims 1-20, or a pharmaceutically acceptable salt, solvate, stereoisomer, or tautomer thereof, wherein Z is —N(R)₂.

31. The compound of any one of claims 1-20, or a pharmaceutically acceptable salt, solvate, stereoisomer, or tautomer thereof, wherein Z is C₁₋₁₀ alkoxy.

32. The compound of any one of claims 1-20, or a pharmaceutically acceptable salt, solvate, stereoisomer, or tautomer thereof, wherein Z is —OH.

33. The compound of any preceding claim, wherein X is hydroxy.

34. The compound of any preceding claim, wherein T is —NH₂.

35. The compound of any preceding claim, wherein both Y and Y¹ are N.

36. A pharmaceutical composition comprising a pharmaceutically acceptable excipient and an effective amount of a compound of any one of claims 1-3, or a pharmaceutically acceptable salt, solvate, stereoisomer, or tautomer thereof.

37. A compound or a pharmaceutically acceptable salt, solvate, stereoisomer, or tautomer thereof, selected from Table 1, Table 1A, or Table 2.

38. A method for modulating or degrading protein which is expressed from the SMARCA2 gene, which method comprises contacting the protein with an effective amount of a compound according to any one of claims 1-3.

39. A method for modulating or degrading protein which is expressed from the SMARCA4 gene, which method comprises contacting the protein with an effective amount of a compound according to any one of claims 1-3.

40. A method for modulating or degrading protein which is expressed from the SMARCA2 gene in a subject, which method comprises administering to said subject an effective amount of a compound according to any one of claims 1-3.

41. A method for modulating or degrading protein which is expressed from the SMARCA4 gene in a subject, which method comprises administering to said subject an effective amount of a compound according to any one of claims 1-3.

42. A method for modulating or degrading protein which is expressed from the SMARCA2 gene in a subject, which method comprises administering to said subject an effective amount of a pharmaceutical composition according to claim 36.

43. A method for modulating or degrading protein which is expressed from the SMARCA4 gene in a subject, which method comprises administering to said subject an effective amount of a pharmaceutical composition according to claim 36.

44. A method for treating cancer in a subject in need thereof, which method comprises administering to said subject an effective amount of a compound according to any one of claims 1-3.

45. A method for treating cancer in a subject in need thereof, which method comprises administering to said subject an effective amount of a pharmaceutical composition according to claim 36.

46. A method for treating hyperplasias in a subject in need thereof, which method comprises administering to said subject an effective amount of a compound according to claim 1.

47. A method for treating hyperplasias in a subject in need thereof, which method comprises administering to said subject an effective amount of a pharmaceutical composition according to claim 36.
